(19) United States
(12) Patent Application Publication
(10) Pub. No.: US 2026/0184687 A1
(43) Pub. Date: Jul. 2, 2026

(54) DIHYDROINDENE DERIVATIVES AS MALT1 INHIBITORS

(71) Applicant: C4X Discovery Limited, Manchester (GB)

(72) Inventors: Ian Duncan Linney, London (GB); Nicholas Charles Ray, London (GB); Oliver Thomas Kern, London (GB); Martin John Watson, Manchester (GB); Paul Matthew Blaney, Manchester (GB); Grzegorz K. Zielinski, Krakow (PL); Simon Osborne, London (GB); Martin Ambler, London (GB)

(21) Appl. No.: 18/864,982

(22) PCT Filed: May 12, 2023
(86) PCT No.: PCT/GB2023/051246

§ 371 Date: Nov. 12, 2024

(30) Foreign Application Priority Data
May 13, 2022 (GB) 2207050.2

(51) Int. Cl. C07D 335/02; A61K 31/165; A61K 31/18; A61K 31/197; A61K 31/351; A61K 31/382; A61K 31/4015; A61K 31/4166; A61K 31/421; A61K 31/4245; A61K 31/44; A61K 31/4412; A61K 31/4427; A61K 31/443; A61K 31/4436; A61K 31/4439; A61K 31/444; A61K 31/445; A61K 31/4465; A61K 31/4545; A61K 31/496; A61K 31/506; A61K 31/537; A61K 31/5375; A61K 31/5377; A61K 31/54; A61K 31/541; C07C 233/43; C07C 235/10; C07C 237/22; C07C 311/14; C07C 311/46; C07D 205/04; C07D 207/27; C07D 211/46; C07D 211/48; C07D 211/62; C07D 213/74; C07D 213/81; C07D 233/72; C07D 263/34; C07D 271/08; C07D 295/215; C07D 309/08; C07D 401/12; C07D 405/14; C07D 409/12; C07D 413/12; C07D 413/14; C07D 417/12; C07D 453/02

(52) U.S. Cl. CPC C07D 335/02; A61K 31/165; A61K 31/18; A61K 31/197; A61K 31/351; A61K 31/382; A61K 31/4015; A61K 31/4166; A61K 31/421; A61K 31/4245; A61K 31/44; A61K 31/4412; A61K 31/4427; A61K 31/443; A61K 31/4436; A61K 31/4439; A61K 31/444; A61K 31/445; A61K 31/4465; A61K 31/4545; A61K 31/496; A61K 31/506; A61K 31/537; A61K 31/5375; A61K 31/5377; A61K 31/54; A61K 31/541; C07C 233/43; C07C 235/10; C07C 237/22; C07C 311/14; C07C 311/46; C07D 205/04; C07D 207/27; C07D 211/46; C07D 211/48; C07D 211/62; C07D 213/74; C07D 213/81; C07D 233/72; C07D 263/34; C07D 271/08; C07D 295/215; C07D 309/08; C07D 401/12; C07D 405/14; C07D 409/12; C07D 413/12; C07D 413/14; C07D 417/12; C07D 453/02; C07C 2601/02; C07C 2602/08

(57) ABSTRACT

The present invention relates to compounds that are MALT1 inhibitors. The compounds have the structural formula I defined herein. The present invention also relates to processes for the preparation of these compounds, to pharmaceutical compositions comprising them, and to their use in the treatment of diseases or disorders associated with MALT1.

### INTRODUCTION

The present invention relates to compounds that are MALT1 inhibitors. The present invention also relates to processes for the preparation of these compounds, to pharmaceutical compositions comprising them, and to their use in the treatment of diseases or disorders associated with MALT1.

### BACKGROUND OF THE INVENTION

Mucosa-associated lymphoid tissue lymphoma translocation protein 1 (MALT1) is an intracellular protein which plays a key role in antigen receptor-induced NF-kB pathway activation in T and B lymphocytes, via both a scaffolding function and a protease function. Antigen receptor triggering leads to formation of the CBM complex, comprising a CARMA or CARD protein, BCL10 and MALT1, that subsequently acts as a scaffold to recruit the ubiquitin ligase TRAF6 and the kinases TAK1 and IKK. This leads to NF-kB activation through IKK-dependent phosphorylation and proteasomal degradation of the NF-kB inhibitor IkB, allowing NF-kB to translate to the nucleus and initiate transcription of target genes (doi.org/10.1038/ni1568; doi:10.1007/s00018-015-2059-z). Additionally, MALT1 proteolytically cleaves a variety of substrates involved in NF-kB pathway regulation, including RelB, A20, CYLD, regnase-1, HOIL, BCL10 and NIK; as well as autoproteolytic cleavage (doi:10.1016/j.biochi.2015.09.018). The overall effect of these cleavage events is thought to be expansion of the amplitude and duration of the NF-kB response (doi:10.1016/j.biochi.2015.09.018).

Several lines of genetic evidence suggest a key role of MALT1 in the immune response. Mice lacking MALT1 protein were viable, but showed impairment in the generation and activation of T_{reg} cells and less activated T cells in the periphery. MALT1 KO mice were protected in inflammatory models for MS (EAE) and rheumatoid arthritis (doi:10.4049/jimmunol.1201351). Mice expressing proteolytically inactive MALT1 show defects in multiple immune cell types including mature T- and B-cells and T_{reg} cells, and develop progressive multiorgan inflammatory pathology (doi:10.4049/jimmunol.1402254; doi:10.3389/fimmu.2020.00745). A small cohort of human patients with defective MALT1 expression and/or function have presented with combined immunodeficiency (doi:10.1007/s10875-014-0125-1; doi:10.1016/j.jaci.2013.04.047).

The highly aggressive activated B-cell (ABC) subtype of diffuse large B-cell lymphoma (DLBCL) is dependent on NF-kB activation via constitutive activation of the CARMA1-BCL10-MALT1 pathway for its survival and proliferation (doi.org/10.1038/nature04687). This constitutive NF-kB activation occurs often due to a variety of oncogenic mutations in pathway genes, including CARD11, MYD88, CD79A/B and A20 (doi:10.1038/nature07968; doi:10.1016/j.ccr.2012.11.003). Pre-clinical studies have suggested that MALT1 protease inhibition may be an effective treatment rationale for ABC-DLBCL, as treatment with the MALT1 inhibitor z-VRPR-fmk decreased the expression of NF-kB target genes with concomitant reduction in cell growth and viability (doi:10.1073/pnas.0907511106; doi:10.1084/jem.20091167), and small-molecule MALT1 inhibitors have also been shown to be active in xenograft models of ABC-DLBCL (doi:10.1016/j.ccr.2012.11.002; doi:10.1016/j.ccr.2012.11.003). Chronic activation of the BCR-mediated CBM-NF-kB pathway has been identified in a subset of mantle cell lymphoma (MCL) lines, suggesting that a subset of MCL may also be responsive to MALT1 inhibition (doi:10.1038/nm.3435). In MALT lymphoma (a type of non-Hodgkin lymphoma (NHL), the fusion protein cIAP2-MALT1 leads to constitutive activation of the NF-kB pathway, and these patients may also benefit from treatment with MALT1 inhibitors (doi 10.1074/jbc.M605116200). MALT1 inhibition may play a role in the treatment of some solid tumour types such as glioblastoma, breast cancer, melanoma, lung cancer, prostate cancer, pancreatic cancer and osteosarcoma (doi:10.1038/s41388-019-0958-4; doi:10.15252/embj.2019102030; doi:10.1111/jcmm.15383; doi:10.1038/oncsis.2017.68; doi:10.1038/onc.2015.146; doi:10.3390/biomedicines9030250; doi:10.1002/ijc.32567).

BTK inhibitors such as ibrutinib are important therapies for cancers such as MCL and chronic lymphocytic leukemia (CLL), but effectiveness is limited due to primary or acquired resistance (doi:10.3390/cancers12051328). MALT1 sits downstream of BTK in the NF-kB activation pathway and therefore may be an effective target either in combination with BTK inhibitors, or in BTKi-refractive tumours. MALT1 has been shown to be constitutively active in CLL cell lines and treatment with the MALT-1 inhibitor MI-2 is effective against both naïve and ibrutinib-resistant cell lines (doi:10.1158/0008-5472.CAN-17-2485). MALT1 inhibition has also been shown to be synergistic with the mTORC1 inhibitor, rapamycin, in ABC-DLBCL cell lines, PDX and in vivo models, opening further possibilities for combination treatment and mitigation strategies for MALT1i resistance (doi:10.1182/blood.2019004713). Combinations of MALT1 inhibitors together with inhibitors of the Bcl-2 family protein have also been demonstrated to show a synergistic benefit in animal models of B-cell lymphoma (WO2023/016995).

MALT1 inhibitors have also been proposed to be effective therapies for a range of cancers, independent of dysregulation of the NF-kB pathway, as an immunomodulatory therapy (doi:10.1038/s41586-019-1215-2; WO2018/226150 & WO2018/141749). Genetic evidence from MALT1-deficient mice suggests that MALT1 promotes development of T_{reg} cells in vivo, which in turn inhibit several types of immune cells, suppressing the anti-tumour immune response. Further studies targeting the CBM complex (by either MALT1 inhibition or CARMA1 deletion in T_{reg} cells) led to a gain of effector activity by T_{reg} cells and enhanced control of tumour growth. A MALT1 inhibitor synergized with anti-PD1 therapy in both poorly immunogenic and immunogenic murine melanoma models (doi:10.1038/s41586-019-1215-2), suggesting a possible combinatorial role of MALT1 inhibitors with immunotherapies, including anti-PD1, anti-PD-L1 and anti-CTLA4.

As well as having a use in oncology indications, small-molecule MALT1 inhibitors may also be effective therapies in inflammatory disorders, for example multiple sclerosis, psoriasis, ulcerative colitis and rheumatoid arthritis. MI-2 has been shown to suppress the differentiation of monocytes into osteoclasts in the presence or absence of TNFa, and to ameliorate the pathologic bone erosion and synovitis in a mouse collagen-induced arthritis (CIA) model, suggesting a role for MALT1 inhibitors in the treatment of rheumatoid arthritis (doi:10.1038/s41598-017-12349-9). The scaffold protein CARD14 forms a signalling complex with BCL10 and MALT1 in keratinocytes and this process is enhanced upon pathogenic CARD14 mutation which as in turn been linked to susceptibility to psoriasis (doi:10.1016/j.jid.2016.09.031). MALT1 inhibitors have also been successfully tested in mouse models of multiple sclerosis and ulcerative colitis (doi:10.1186/1742-2094-11-124). Pharmacological inhibition of MALT1 protease activity has been shown to suppress endothelial activation through increasing MCPIP1 expression, inhibiting TNFa-induced VCAM-1 expression in HUVECs and LPS-induced VCAM-1 expression in mice, suggesting a possible role for MALT1 inhibitors in the treatment of vascular inflammatory diseases such as atherosclerosis (doi:10.1016/j.cellsig.2018.05.009).

Homozygous MALT1 W580S mutations have been determined in patients with combined immunodeficiency (CID). This was associated with low MALT1-W580S protein levels leading to reduced amplitude but extended duration of NF-kB due to impaired negative feedback through reduced HOIL1 cleavage, manifesting clinically in increased dermal and GI inflammation and increased susceptibility to infection (doi.org/10.1016/j.jaci.2013.10.045). Small-molecule ligands known to bind to the MALT1 wild-type allosteric site have been shown to also bind to the W580S mutant leading to enzyme stabilization and reconstitution of protein levels, even after ligand washout. Both scaffolding and protease functions were restored; thus allosteric MALT1 inhibitors may be able to serve as ‘molecular correctors’ in cases of W80S MALT1 mutations (doi.org/10.1038/s41589-018-0222-1).

Clinical and pre-clinical studies are ongoing with a variety of MALT1 inhibitors. JNJ-67856633 is a small molecule MALT1 inhibitor currently in PhI studies in patients with NHL and CLL. A review of MALT1 inhibitor patents has recently been published (doi:10.1080/13543776.2021.1951703).

Therefore, there is an ongoing need for agents capable of MALT1 inhibition, given the role of MALT1 in multiple indications.

### SUMMARY OF THE INVENTION

In one aspect, the present invention provides a compound, or a pharmaceutically acceptable salt thereof as defined herein.

In another aspect, the present invention provides a pharmaceutical composition comprising a compound of the invention as defined herein, or a pharmaceutically acceptable salt thereof, and one or more pharmaceutically acceptable excipients.

In another aspect, the present invention relates to a compound of the invention as defined herein, or a pharmaceutically acceptable salt thereof, or a pharmaceutical composition as defined herein, for use in therapy.

In another aspect, the present invention relates to a compound of the invention as defined herein, or a pharmaceutically acceptable salt thereof, or a pharmaceutical composition as defined herein, for use in the treatment of diseases or disorders mediated by MALT1.

In another aspect, the present invention relates to a method of treating a disease or disorder mediated by MALT1, said method comprising administering to a subject in need of such treatment a therapeutically effective amount of a compound of the invention as defined herein, or a pharmaceutically acceptable salt thereof, or a pharmaceutical composition as defined herein.

Examples of diseases or disorders mediated by MALT1 include:
i) lymphomas, leukaemias, carcinomas, and sarcomas; such as non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukaemia (CLL), small lymphocytic lymphoma (SLL), Waldenström macroglobulinemia, lymphoblastic T cell leukaemia, chronic myelogenous leukaemia (CML), hairy-cell leukaemia, acute lymphoblastic T cell leukaemia, plasmacytoma, immunoblastic large cell leukaemia, megakaryoblastic leukaemia, acute megakaryocytic leukaemia, promyelocytic leukaemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, oesophageal cancer, salivary gland cancer, nasopharyngeal cancer, buccal cancer, cancer of the mouth, or GIST (gastrointestinal stromal tumour); and
ii) immunological diseases including autoimmune and inflammatory disorders; such as arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, or polymyositis.

In another aspect, the present invention provides a compound, or a pharmaceutically acceptable salt thereof, or a pharmaceutical composition as defined herein, for use in the treatment of non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, chronic lymphocytic leukaemia (CLL), or small lymphocytic lymphoma (SLL).

In another aspect, the present invention provides a method of treating non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, chronic lymphocytic leukaemia (CLL), or small lymphocytic lymphoma (SLL), said method comprising administering to a subject in need of such treatment a therapeutically effective amount of a compound, or a pharmaceutically acceptable salt thereof, or a pharmaceutical composition as defined herein.

The present invention further provides a method of synthesising a compound, or a pharmaceutically acceptable salt thereof, as defined herein.

In another aspect, the present invention provides a compound, or a pharmaceutically acceptable salt thereof, obtainable by, or obtained by, or directly obtained by a method of synthesis as defined herein.

In another aspect, the present invention provides novel intermediates as defined herein which are suitable for use in any one of the synthetic methods set out herein.

Preferred, suitable, and optional features of any one particular aspect of the present invention are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

In this specification the term “alkylene” includes both straight and branched chain divalent alkyl groups. For example, “C₁₋₄alkylene” includes methylene (—CH₂—), ethylene (—CH₂CH₂—), propylene and butylene.

In this specification the term “alkoxy” includes both straight and branched chain alkyl groups singularly bonded to oxygen. For example, “C₁₋₄alkoxy” includes methoxy, ethoxy, iso-propoxy and t-butoxy.

The term “Cm-n” or used alone or as a prefix, refers to any group having m to n carbon atoms.

“Cycloalkyl” means a hydrocarbon monocyclic or bicyclic ring containing carbon atoms. Examples of monocyclic cycloalkyl groups include cyclopropyl, cyclobutyl, cyclopentyl, cyclohexyl, cycloheptyl. Bicyclic rings may be fused or spiro attached; examples of bicyclic cycloalkyl groups include bicyclo[2.2.2]octane, bicyclo[2.1.1]hexane, bicyclo[1.1.1]pentane, spiro[2.4]heptane, bicyclo[4.1.0]heptane and bicyclo[2.2.1]heptane.

The term “halo” refers to fluoro, chloro, bromo and iodo.

The term “haloalkyl” is used herein to refer to an alkyl group respectively in which one or more hydrogen atoms have been replaced by halogen (e.g. fluorine) atoms. Examples of haloalkyl groups include fluoroalkyl groups such as —CHF₂, —CH₂CF₃, or perfluoroalkyl/alkoxy groups such as —CF₃, or —CF₂CF₃.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, dihydroisoxazolyl (such as 4,5-dihydroisoxazolyl), dihydropyridinyl (such as 1,2-dihydropyridinyl or 1,6-dihydropyridinyl), tetrahydro-oxazolyl, tetrahydro-oxadiazolyl, tetrahydro-dioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. Examples of bridged bicyclic heterocyclic ring systems include quinuclidine and 3-thiabicyclo[3.1.0]hexane. Examples of spiro bicyclic heterocyclic ring systems include 1-azaspiro[3.3]heptane, 6-oxa-8-azaspiro[3.5]nonane, 5-azaspiro[3.4]octane, and 7-oxa-5-azaspiro[3.4]octane. As the skilled person would appreciate, any heterocycle may be linked to another group via any suitable atom, such as via a carbon or nitrogen atom. Suitably, the term “heterocyclyl”, “heterocyclic” or “heterocycle” will refer to 4, 5, 6 or 7 membered monocyclic rings as defined above.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five. Suitably, the term “heteroaryl” or “heteroaromatic” will refer to 5 or 6 membered monocyclic heteroaryl rings as defined above.

Non-limiting examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzothienyl, dihydrobenzofuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl, 3,4-dihydro-2H-pyrido[3,2-b][1,4]oxazinyl, 4,5,6,7-tetrahydrobenzo[d]isoxazolyl, 4,5,6,7-tetrahydro-[1,2,3]triazolo[1,5-a]pyridinyl, 5,6-dihydro-8H-[1,2,4]triazolo[3,4-c][1,4]oxazinyl, 5,6-dihydro-4H-pyrrolo[1,2-c][1,2,3]triazolyl, 6,7-dihydro-5H-pyrrolo[2,1-c][1,2,4]triazolyl, 5,6,7,8-tetrahydro-[1,2,4]triazolo[4,3-a]pyridinyl, 6,7-dihydro-4H-[1,2,3]triazolo[5,1-c][1,4]oxazinyl and 1,4,5,6-tetrahydrocyclopenta[d][1,2,3]triazol-5-yl.

Non-limiting examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Non-limiting examples of six membered heteroaryl groups include but are not limited to pyridyl, pyrazinyl, pyridazinyl, pyrimidinyl and triazinyl.

Particular non-limiting examples of bicyclic heteroaryl groups containing a six membered ring fused to a five membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl, pyrrolopyridine, and pyrazolopyridinyl groups.

Particular non-limiting examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

Particular non-limiting examples of bicyclic heteroaryl groups containing a five membered ring fused to a five membered ring include but are not limited to 6,7-dihydro-5H-pyrrolo[2,1-c][1,2,4]triazolyl, 5,6-dihydro-4H-pyrrolo[1,2-c][1,2,3]triazolyl and 1,4,5,6-tetrahydrocyclopenta[d][1,2,3]triazol-5-yl.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In this particular embodiment, an aryl is phenyl or naphthyl, especially phenyl.

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In a first aspect, the present invention provides a compound of Formula I, or a pharmaceutically acceptable salt thereof:

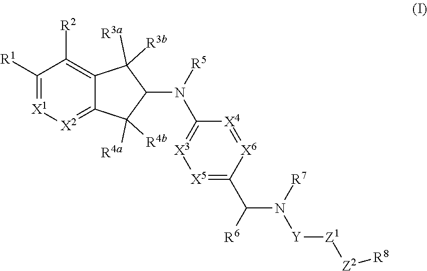

wherein:
X¹ and X² are independently selected from CR⁹ or N;
X³, X⁴, X⁵, and X⁶ are independently selected from CR¹⁰ or N, provided that no more than two of X³, X⁴, X⁵, and X⁶ are N;
Y is

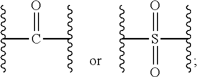

Z¹ is absent,

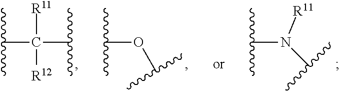

Z² is absent or a C₁₋₄alkylene linker group, optionally substituted with one or more substituents independently selected from C₁₋₄alkyl, C₁₋₄alkoxy, C₁₋₄haloalkyl, halo, cyano, hydroxy, and C₃₋₇cycloalkyl; and optionally spiro attached, to a 3- to 6-membered cycloalkyl or 3- to 6-membered heterocyclyl ring, wherein the cycloalkyl and heterocyclyl ring is optionally substituted with one or more substituents independently selected from C₁₋₄alkyl, C₁₋₄haloalkyl, hydroxy, C₁₋₄alkoxy, C₃₋₆cycloalkyl, halo, and cyano;
R¹ and R² are independently selected from hydrogen, C₁₋₄alkyl, C₁₋₄alkoxy, C₁₋₄haloalkyl, C₁₋₄haloalkoxy, halo, cyano, hydroxy, C₃₋₇cycloalkyl, and S(O)₂C₁₋₄alkyl;
R^{3a}, R^{3b}, R^{4a}, and R^{4b} are independently selected from hydrogen, C₁₋₄alkyl, C₁₋₄haloalkyl, C₃₋₇cycloalkyl, halo, cyano, NR¹³R¹⁴, C(O)OR¹⁵, C(O)NR¹⁶R¹⁷, and OR¹⁸;
R⁵ is hydrogen or C₁₋₃alkyl;
R⁶ is C₁₋₃alkyl, C₁₋₃haloalkyl, or C₃₋₅cycloalkyl;
R⁸ is hydrogen, C₁₋₃alkyl, or CD₃;
R⁸ is selected from OR¹⁹, NR²⁰R²¹, C(O)R²², S(O)₂NR²³R²⁴, cyano, C₃₋₈cycloalkyl, 4- to 10-membered heterocyclyl, 5- to 10-membered heteroaryl, and phenyl, wherein said C₃₋₈cycloalkyl, 4- to 10-membered heterocyclyl, 5- to 10-membered heteroaryl, and phenyl groups are optionally substituted with one or more substituents of formula C₀₋₃alkylene-R²⁵, wherein each R²⁵ is independently selected from C₁₋₄alkyl, hydroxy, C₁₋₄alkoxy, halo, C₁₋₄haloalkyl, C₃₋₆cycloalkyl, 4- to 6-membered heterocyclyl, NR²⁶R²⁷, NR²⁸C(O)R²⁹, C(O)R³⁰, S(O)₂R³¹ and NR³²S(O)₂R³³; and
wherein said C₃₋₈cycloalkyl and 4- to 10-membered heterocyclyl R⁸ groups may be:
   i) monocyclic or polycyclic ring systems wherein the rings in the polycyclic ring systems are fused, bridged or spiro attached; and
   ii) in addition to the optional C₀₋₃alkylene-R²⁵ substituents, may also be optionally substituted with one or more oxo groups;
R⁹ is selected from hydrogen, C₁₋₄alkyl, C₁₋₄alkoxy, C₁₋₄haloalkyl, C₁₋₄haloalkoxy, halo, cyano, hydroxy, C₃₋₇cycloalkyl, and S(O)₂C₁₋₄alkyl;
R¹⁰ is selected from hydrogen, C₁₋₄alkyl, C₁₋₄alkoxy, C₁₋₄haloalkyl, halo, cyano, and hydroxy;
R¹¹ and R¹² are independently selected from hydrogen, C₁₋₄alkyl, and halo;
R¹³, R¹⁴, R¹⁵, R¹⁶, and R¹⁷ are independently selected from hydrogen and C₁₋₄alkyl;
R¹⁸ and R¹⁹ are independently selected from hydrogen, C₁₋₄alkyl, C₃₋₇cycloalkyl, 4- to 7-membered heterocyclyl, and 5- or 6-membered heteroaryl, wherein said C₁₋₄alkyl, C₃₋₇cycloalkyl, 4- to 7-membered heterocyclyl, and 5- or 6-membered heteroaryl groups are optionally substituted with one or more substituents selected from C₁₋₄alkyl, halo, OH, C₁₋₄alkoxy, C₃₋₇cycloalkyl, NR³⁴R³⁵, C(O)OR³⁶, and C(O)NR³⁷R³⁸;
R²⁰ and R²¹ are independently selected from hydrogen, C₁₋₄alkyl, C₁₋₄haloalkyl, C₃₋₇cycloalkyl, C(O)C₁₋₄alkyl, C(O)NR³⁹R⁴⁰, and S(O)₂R⁴¹;
R²² is selected from hydroxy, C₁₋₄alkoxy, and NR⁴²R⁴³;
R²³ and R²⁴ are independently selected from hydrogen and C₁₋₄alkyl;
R²⁶ and R²⁷ are independently selected from hydrogen, C₁₋₄alkyl, C₁₋₄haloalkyl, and C₃₋₇cycloalkyl;
R²⁸ is hydrogen or C₁₋₄alkyl;
R²⁹ is C₁₋₄alkyl, C₁₋₄haloalkyl or NR⁴⁴R⁴⁵;
R³⁰ is selected from C₁₋₄alkyl, hydroxy, C₁₋₄alkoxy, NR⁴⁶R⁴⁷, and 4- to 6-membered heterocyclyl;
R³¹ is C₁₋₄alkyl or NR⁴⁸R⁴⁹; and
R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, and R⁴⁹ are independently selected from hydrogen and C₁₋₄alkyl; or
R³⁴ and R³⁵; R³⁷ and R³⁸; R³⁹ and R⁴⁰; R⁴² and R⁴³; R⁴⁴ and R⁴⁵; R⁴⁶ and R⁴⁷; or R⁴⁸ and R⁴⁹, together with the respective nitrogen atom to which they are attached, form a 4- to 7-membered heterocyclic ring.

Particular compounds of the invention include, for example, compounds of the formula I, or pharmaceutically acceptable salts thereof, wherein, unless otherwise stated, each of X¹, X², X³, X⁴, X⁵, X⁶, Y, Z¹, Z², R¹, R², R^{3a}, R^{3b}, R^{4a}, R^{4b}, R⁵, R⁸, R⁷, R⁸, R⁹, R¹⁸, R¹⁹, R²⁰, and R²¹ has any of the meanings defined hereinbefore or in any of paragraphs (1) to (67) hereinafter. For the avoidance of doubt, the scope of the present invention encompasses compounds of formula I, or pharmaceutically acceptable salts thereof, wherein any of the substituent definitions defined herein may be combined with any of the other substituent definitions also defined herein:
(1) X¹ is CH or N;
(2) X¹ is CR⁹, such as CH;
(3) X² is CH or N;
(4) X² is CR⁹, such as CH;
(5) X³, X⁴, X⁵, and X⁶ are independently selected from CH or N, provided that no more than two of X³, X⁴, X⁵, and X⁶ are N;
(6) one of X³, X⁴, X⁵, and X⁶ is N and the other three are CH;
(7) X³ is N and X⁴, X⁵, and X⁶ are CH;
(8) X⁵ is N and X³, X⁴, and X⁶ are CH;
(9) X³ and X⁶ are N, and X⁴ and X⁵ are CH;
(10) X³ and X⁴ are N, and X⁵ and X⁶ are CH;
(11) X³ and X⁴ are CH, and X⁵ and X⁶ are N;
(12) X³ and X⁵ are N, and X⁴ and X⁶ are CH;
(13) X³, X⁴, X⁵, and X⁶ are all CH;
(14) Y is

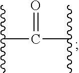 [SMILES: *C(*)=O]

(15) Z¹ is absent,

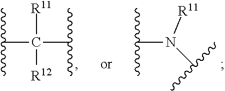

(16) Z¹ is absent, or

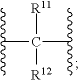

(17) Z¹ is absent;
(18) Z¹ is

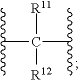

(19) Z¹ is

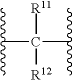

and Z² is absent;
(20) Z¹ is absent and Z² is absent;
(21) Z² is absent or a C₁₋₄alkylene linker group, optionally substituted with one or more substituents independently selected from C₁₋₄alkyl, C₁₋₄alkoxy, C₁₋₄haloalkyl, halo, cyano, hydroxy, and C₃₋₇cycloalkyl;
(22) Z² is absent;
(23) Z² is a C₁₋₃alkylene linker group, optionally substituted with one or more substituents independently selected from C₁₋₄alkyl, C₁₋₄alkoxy, C₁₋₄haloalkyl, halo, cyano, hydroxy, and C₃₋₇cycloalkyl;
(24) Z² is a C₁₋₃alkylene linker group, optionally substituted with one or more substituents independently selected from C₁₋₄alkyl, halo, cyano, hydroxy, and cyclopropyl;
(25) Z² is —CH₂—, —CH₂CH₂—, or —CH₂CH₂CH₂—;
(26) R¹ and R² are independently selected from hydrogen, C₁₋₄alkyl, C₁₋₄alkoxy, C₁₋₄haloalkyl, halo, hydroxy, cyano, and C₃₋₇cycloalkyl;
(27) R¹ and R² are independently selected from hydrogen, methyl, ethyl, methoxy, trifluoromethyl, fluoro, chloro, bromo, cyano and cyclopropyl;
(28) R¹ and R² are independently selected from hydrogen, methoxy, fluoro, chloro, and cyano;
(29) R¹ is selected from methyl, ethyl, methoxy, trifluoromethyl, fluoro, chloro, and cyano, and R² is hydrogen;
(30) R¹ is hydrogen, and R² is selected from methyl, ethyl, methoxy, trifluoromethyl, fluoro, chloro, and cyano;
(31) R¹ and R² are both hydrogen;
(32) R^{3a}, R^{3b}, R^{4a}, and R^{4b} are independently selected from hydrogen, C₁₋₄alkyl, C₁₋₄haloalkyl, C₃₋₇cycloalkyl, halo, and OR¹⁸;
(33) R^{3a}, R^{3b}, R^{4a}, and R^{4b} are independently selected from hydrogen, C₁₋₄alkyl, hydroxy and C₁₋₄alkoxy;
(34) R^{3a}, R^{3b}, R^{4a}, and R^{4b} are independently selected from hydrogen, C₁₋₄alkyl, and C₁₋₄alkoxy;
(35) R^{3a}, R^{3b}, R^{4a}, and R^{4b} are independently selected from hydrogen, methyl, ethyl, and methoxy;
(36) R^{3a}, R^{3b}, R^{4a}, and R^{4b} are all hydrogen;
(37) R^{3a} is C₁₋₄alkyl, such as methyl, and R^{3b}, R^{4a}, and R^{4b} are hydrogen;
(38) R^{3a} is C₁₋₄alkoxy, such as methoxy, and R^{3b}, R^{4a}, and R^{4b} are hydrogen;
(39) R^{3a} and R^{3b} are both C₁₋₄alkyl, such as both methyl;
(40) R^{3a} and R^{3b} are both C₁₋₄alkyl, such as both methyl, and R^{4a} and R^{4b} are both hydrogen;
(41) R^{3a} and R^{4a} are both C₁₋₄alkyl, such as both methyl;
(42) R^{3a} and R^{4a} are both C₁₋₄alkyl, such as both methyl, and R^{3b} and R^{4b} are both hydrogen;
(43) R⁵ is hydrogen or methyl;
(44) R⁵ is hydrogen;
(45) R⁶ is C₁₋₂alkyl, C₁₋₂haloalkyl, or C₃₋₄cycloalkyl;
(46) R⁶ is methyl, C₁₋₂fluoroalkyl, or cyclopropyl;
(47) R⁶ is C₁₋₃alkyl, or C₁₋₃haloalkyl;
(48) R⁶ is methyl;
(49) R⁶ is C₁₋₃haloalkyl;
(50) R⁶ is C₁₋₃fluoroalkyl;
(51) R⁶ is trifluoromethyl;
(52) R⁷ is hydrogen, methyl, or CD₃;
(53) R⁷ is C₁₋₃alkyl;
(54) R⁷ is methyl;
(55) R⁸ is selected from OR¹⁹, NR²⁰R²¹, C(O)R²², S(O)₂NR²³R²⁴, cyano, C₃₋₈cycloalkyl, 4- to 10-membered heterocyclyl, and 5- to 10-membered heteroaryl, wherein said C₃₋₈cycloalkyl, 4- to 10-membered heterocyclyl, and 5- to 10-membered heteroaryl groups are optionally substituted with one or more substituents of formula C₀₋₃alkylene-R²⁵, wherein each R²⁵ is independently selected from C₁₋₄alkyl, hydroxy, C₁₋₄alkoxy, halo, C₁₋₄haloalkyl, C₃₋₆cycloalkyl, 4- to 6-membered heterocyclyl, NR²⁶R²⁷, NR²⁸C(O)R²⁹, C(O)R³⁰, S(O)₂R³¹, and NR³²S(O)₂R³³; and
wherein said C₃₋₈cycloalkyl and 4- to 10-membered heterocyclyl R⁸ groups may be: monocyclic or polycyclic ring systems wherein the rings in the polycyclic ring systems are fused, bridged or spiro attached; and
in addition to the optional C₀₋₃alkylene-R²⁵ substituents, may also be optionally substituted with one or more oxo groups;
(56) R⁸ is selected from OR¹⁹, NR²⁰R²¹, C(O)R²², S(O)₂NR²³R²⁴, cyano, C₃₋₈cycloalkyl, 4- to 10-membered heterocyclyl, and 5- to 6-membered heteroaryl, wherein said C₃₋₈cycloalkyl, 4- to 10-membered heterocyclyl, and 5- to 6-membered heteroaryl groups are optionally substituted with one or more substituents of formula C₀₋₃alkylene-R²⁵, wherein each R²⁵ is independently selected from C₁₋₄alkyl, hydroxy, C₁₋₄alkoxy, halo, C₁₋₄haloalkyl, C₃₋₆cycloalkyl, 4- to 6-membered heterocyclyl, NR²⁶R²⁷, NR²⁸C(O)R²⁹, C(O)R³⁰, S(O)₂R³¹, and NR³²S(O)₂R³³; and
wherein said C₃₋₈cycloalkyl and 4- to 10-membered heterocyclyl R⁸ groups may be: monocyclic or polycyclic ring systems wherein the rings in the polycyclic ring systems are fused, bridged or spiro attached; and
in addition to the optional C₀₋₃alkylene-R²⁵ substituents, may also be optionally substituted with one or more oxo groups;
(57) R⁸ is selected from OR¹⁹, NR²⁰R²¹, C(O)R²², S(O)₂NR²³R²⁴, cyano, C₃₋₈cycloalkyl, 4- to 10-membered heterocyclyl, and 5- to 6-membered heteroaryl, wherein said C₃₋₈cycloalkyl, 4- to 10-membered heterocyclyl, and 5- to 6-membered heteroaryl groups are optionally substituted with one or more substituents of formula C₀₋₃alkylene-R²⁵, wherein each R²⁵ is independently selected from C₁₋₄alkyl, hydroxy, C₁₋₄alkoxy, halo, NR²⁶R²⁷, NR²⁸C(O)R²⁹, C(O)R³⁰, S(O)₂R³¹, and NR³²S(O)₂R³³; and
wherein said C₃₋₈cycloalkyl and 4- to 10-membered heterocyclyl R⁸ groups may be: monocyclic or polycyclic ring systems wherein the rings in the polycyclic ring systems are fused, bridged or spiro attached; and
in addition to the optional C₀₋₃alkylene-R²⁵ substituents, may also be optionally substituted with one or more oxo groups;
(58) R⁸ is selected from C₃₋₈cycloalkyl, 4- to 10-membered heterocyclyl, and 5- to 6-membered heteroaryl, wherein said C₃₋₈cycloalkyl, 4- to 10-membered heterocyclyl, and 5- to 6-membered heteroaryl groups are optionally substituted with one or more substituents of formula C₀₋₃alkylene-R²⁵, wherein each R²⁵ is independently selected from C₁₋₄alkyl, hydroxy, C₁₋₄alkoxy, halo, NR²⁶R²⁷, NR²⁸C(O)R²⁹, C(O)R³⁰, S(O)₂R³¹, and NR³²S(O)₂R³³; and
wherein said C₃₋₈cycloalkyl and 4- to 10-membered heterocyclyl R⁸ groups may be: monocyclic or polycyclic ring systems wherein the rings in the polycyclic ring systems are fused, bridged or spiro attached; and
in addition to the optional C₀₋₃alkylene-R²⁵ substituents, may also be optionally substituted with one or more oxo groups;
(59) R⁸ is selected from azetidine, pyrrolidine, piperidine, piperazine, pyrazolidine, 1,2-dihydropyridine, imidazolidine, oxazolidine, morpholine, tetrahydrofuran, tetrahydropyran, tetrahydrothiopyran, thiomorpholine, quinuclidine, tetrahydrothiazine, thietane, tetrahydrothiophene, 1-azaspiro[3.3]heptane, 6-oxa-8-azaspiro[3.5]nonane, 7-oxa-5-azaspiro[3.4]octane, thiabicyclo[3.1.0]hexane, 5-azaspiro[3.4]octane, and isothiazolidine, optionally substituted with one or more oxo groups and optionally substituted with one or more substituents of formula C₀₋₃alkylene-R²⁵, wherein each R²⁵ is independently selected from C₁₋₄alkyl, hydroxy, C₁₋₄alkoxy, halo, NR²⁶R²⁷, NR²⁸C(O)R²⁹, C(O)R³⁰, S(O)₂R³¹, NR³²S(O)₂R³³, C₃₋₆cycloalkyl, and 4- to 6-membered heterocyclyl;
(60) R⁸ is selected from OR¹⁹, NR²⁰R²¹, C(O)R²², S(O)₂NR²³R²⁴, cyano, or one of the following groups:

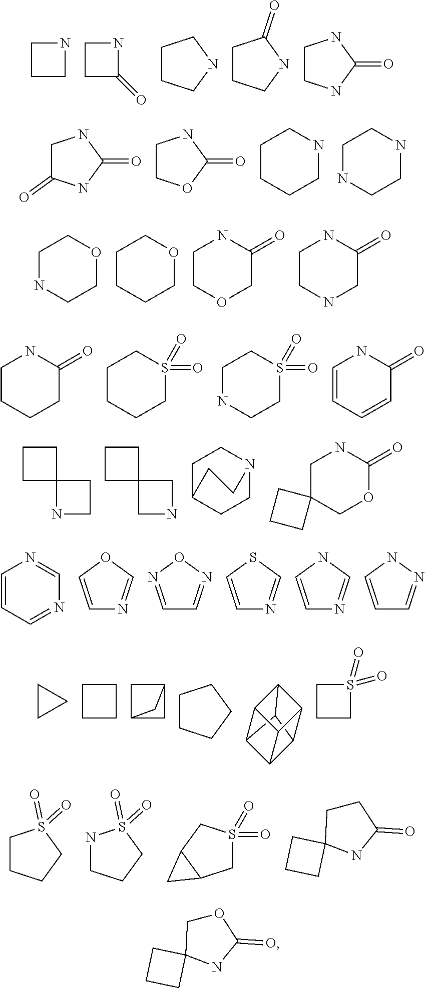

optionally substituted with one or more substituents of formula C₀₋₃alkylene-R²⁵, wherein each R²⁵ is independently selected from C₁₋₄alkyl, hydroxy, C₁₋₄alkoxy, halo, NR²⁶R²⁷, NR²⁸C(O)R²⁹, C(O)R³⁰, S(O)₂R³¹, NR³²S(O)₂R³³, C₃₋₆cycloalkyl, and 4- to 6-membered heterocyclyl;
(61) R⁸ is:

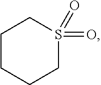

optionally substituted with one or more substituents of formula C₀₋₃alkylene-R²⁵, wherein each R²⁵ is independently selected from C₁₋₄alkyl, hydroxy, C₁₋₄alkoxy, halo, NR²⁶R²⁷, NR²⁸C(O)R²⁹, C(O)R³⁰, S(O)₂R³¹, and NR³²S(O)₂R³³.
(62) R⁹ is selected from hydrogen, C₁₋₄alkyl, C₁₋₄haloalkoxy, and halo;
(63) R⁹ is selected from hydrogen, methyl, difluoromethoxy, fluoro, chloro and bromo;
(64) R¹⁸ and R¹⁹ are independently selected from hydrogen and C₁₋₄alkyl;
(65) R¹⁸ and R¹⁹ are independently selected from hydrogen, methyl, and ethyl;
(66) R²⁰ and R²¹ are independently selected from hydrogen, C₁₋₄alkyl, and C(O)C₁₋₄alkyl;
(67) R²⁰ and R²¹ are independently selected from hydrogen, methyl, and C(O)Me;

Suitably, X¹ is as defined in any one of paragraphs (1) to (2) above.

Suitably, X² is as defined in any one of paragraphs (3) to (4) above.

Suitably, X³ to X⁶ are as defined in any one of paragraphs (5) to (13) above. More suitably, X³ to X⁶ are as defined in paragraphs (7) to (8) above.

Suitably, Y is as defined in paragraph (14) above.

Suitably, Z¹ is as defined in any one of paragraphs (15) to (20) above. More suitably, Z¹ is as defined in paragraph (17) above.

Suitably, Z² is as defined in any one of paragraphs (21) to (25) above. More suitably, Z² is as defined in paragraph (22) or (25) above.

Suitably, R¹ and R² are as defined in any one of paragraphs (26) to (31) above. In an embodiment, R¹ and R² are as defined in paragraph (31) above.

Suitably, R^{3a} to R^{4b} are as defined in any one of paragraphs (32) to (42) above. In an embodiment, R^{3a} to R^{4b} are as defined in paragraphs (39) to (40) above.

Suitably, R⁵ is as defined in any one of paragraphs (43) to (44) above. In an embodiment, R⁵ is as defined in paragraph (44) above.

Suitably, R⁶ is as defined in any one of paragraphs (45) to (51) above. In an embodiment, R⁶ is as defined in paragraph (51) above.

Suitably, R⁷ is as defined in any one of paragraphs (52) to (54) above. In an embodiment, R⁷ is as defined in paragraph (54) above.

Suitably, R⁸ is as defined in any one of paragraphs (55) to (61) above. In an embodiment, R⁸ is as defined in paragraph (61) above.

Suitably, R⁹ is as defined in any one of paragraphs (62) to (63) above. In an embodiment, R⁹ is as defined in paragraph (63) above.

Suitably, R¹⁸ and R¹⁹ are as defined in any one of paragraphs (64) to (65) above.

Suitably, R²⁰ and R²¹ are as defined in any one of paragraphs (66) to (67) above.

In a convenient embodiment, the compound of formula I is a compound according to formula IA (sub-structure of formula I) below, or a pharmaceutically acceptable salt thereof:

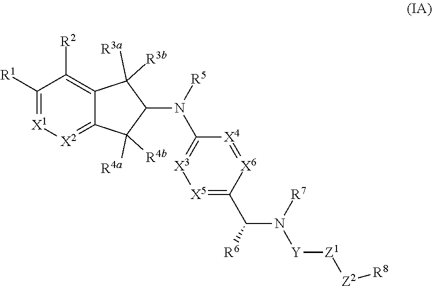

In a further group of compounds, the compounds have one of the structural formulae IB to IS (sub-structures of formula I) shown below:

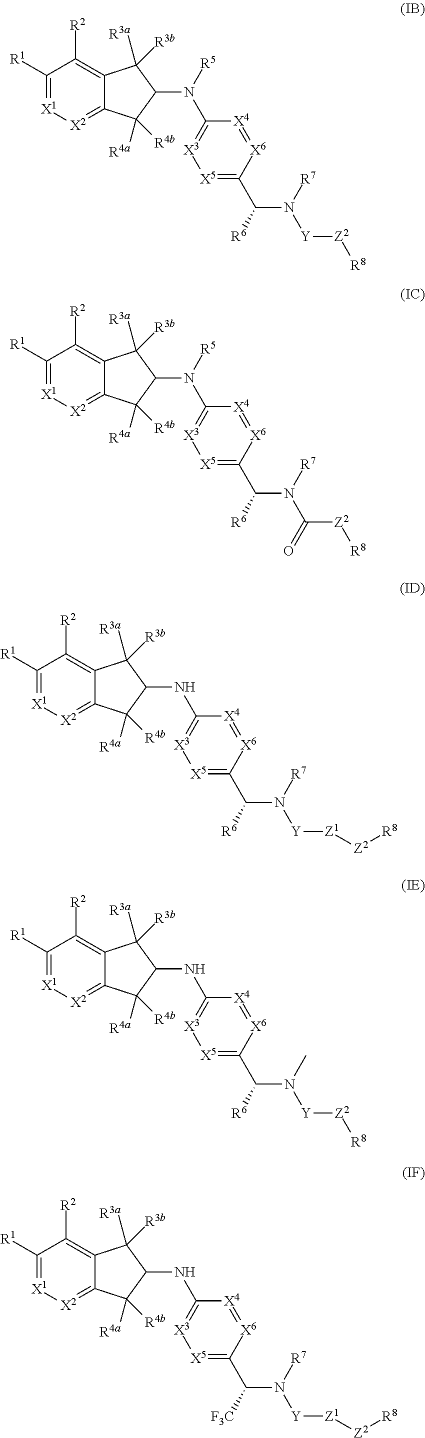

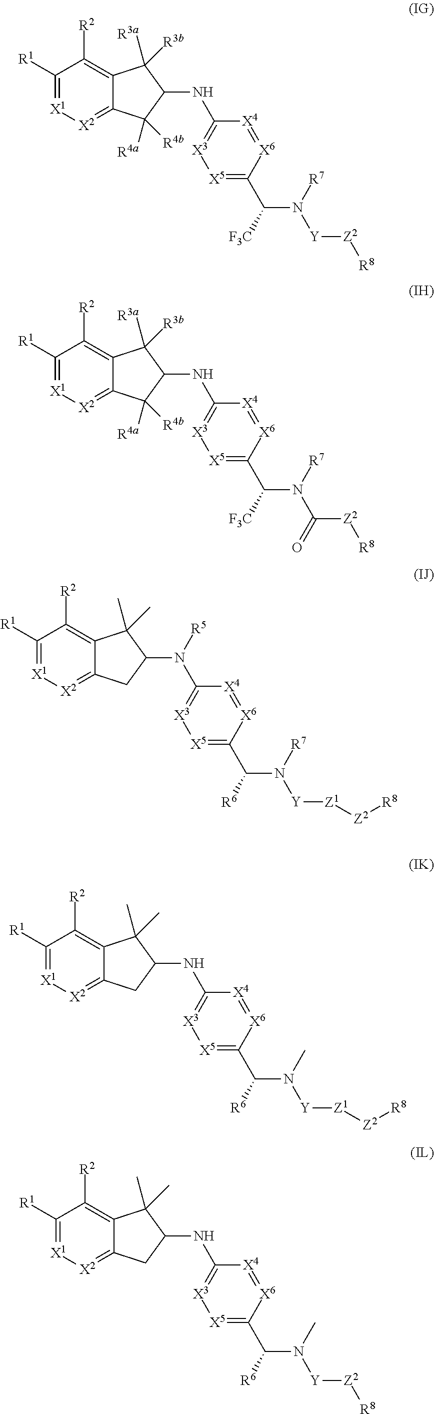

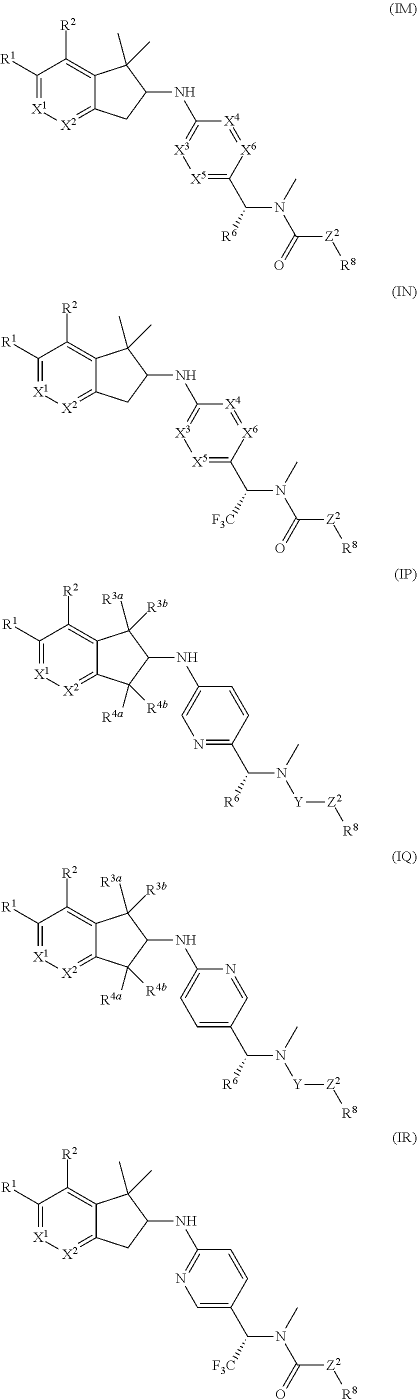

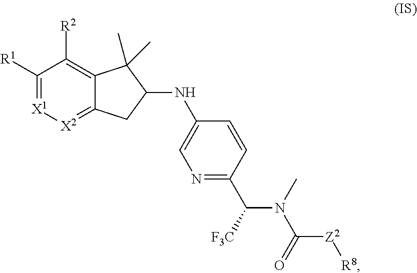

wherein X¹, X², X³, X⁴, X⁵, X⁶, Y, Z¹, Z², R¹, R², R^{3a}, R^{3b}, R^{4a}, R^{4b}, R⁵, R⁶, R⁷, and R⁸ are as defined hereinbefore.

In a further group of compounds, the compounds have one of the structural formulae IA to IS shown above, wherein X¹ is as defined in any one of paragraphs (1) to (2) above; X² is as defined in any one of paragraphs (3) to (4) above; X³ to X⁶ are as defined in any one of paragraphs (5) to (13) above; Y is as defined in paragraph (14) above; Z¹ is as defined in any one of paragraphs (15) to (20) above; Z² is as defined in any one of paragraphs (21) to (25) above; R¹ and R² are as defined in any one of paragraphs (26) to (31) above; R^{3a} to R^{4b} are as defined in any one of paragraphs (32) to (42) above; R⁵ is as defined in any one of paragraphs (43) to (44) above; R⁶ is as defined in any one of paragraphs (45) to (51) above; R⁷ is as defined in any one of paragraphs (52) to (54) above; and R^{a} is as defined in any one of paragraphs (55) to (61) above.

Particular compounds of the present invention include any one of the following:
N—((S)-1-(4-((-1,1-dimethyl-2,3-dihydro-1H-inden-2-yl)amino)phenyl)-2,2,2-trifluoroethyl)-N-methyltetrahydro-2H-thiopyran-4-carboxamide 1,1-dioxide;
(S)—N-(1-(4-((2,3-dihydro-1H-inden-2-yl)amino)phenyl)-2,2,2-trifluoroethyl)-N-methyltetrahydro-2H-thiopyran-4-carboxamide 1,1-dioxide;
N-((1S)-1-(4-((1,1-dimethyl-2,3-dihydro-1H-inden-2-yl)amino)phenyl)-2,2,2-trifluoroethyl)-N-methyloxazole-5-carboxamide;
1-acetyl-N-((1S)-1-(4-((1,1-dimethyl-2,3-dihydro-1H-inden-2-yl)amino)phenyl)-2,2,2-trifluoroethyl)-N-methylazetidine-3-carboxamide;
N-Methyl-N—((S)-2,2,2-trifluoro-1-(4-(((1,2-cis)-1-methyl-2,3-dihydro-1H-inden-2-yl)amino)phenyl)ethyl)tetrahydro-2H-thiopyran-4-carboxamide 1,1-dioxide;
N-Methyl-N—((S)-2,2,2-trifluoro-1-(4-(((1,2-trans)-1-methyl-2,3-dihydro-1H-inden-2-yl)amino)phenyl)ethyl)tetrahydro-2H-thiopyran-4-carboxamide 1,1-dioxide;
N-methyl-N-((1S)-2,2,2-trifluoro-1-(4-((1-methoxy-2,3-dihydro-1H-inden-2-yl)amino)phenyl)ethyl)tetrahydro-2H-thiopyran-4-carboxamide 1,1-dioxide;
N—((S)-1-(4-(((S)-1,1-dimethyl-2,3-dihydro-1H-inden-2-yl)amino)phenyl)-2,2,2-trifluoroethyl)-N-methylmorpholine-4-carboxamide;
N—((S)-1-(4-(((R)-1,1-dimethyl-2,3-dihydro-1H-inden-2-yl)amino)phenyl)-2,2,2-trifluoroethyl)-N-methylmorpholine-4-carboxamide;
N-((1S)-1-(4-((1,1-dimethyl-2,3-dihydro-1H-inden-2-yl)amino)phenyl)-2,2,2-trifluoroethyl)-N-methylthiomorpholine-4-carboxamide 1,1-dioxide;
N-((1S)-1-(4-((1,1-dimethyl-2,3-dihydro-1H-inden-2-yl)amino)phenyl)-2,2,2-trifluoroethyl)-4-hydroxy-N,4-dimethylpiperidine-1-carboxamide;
N-((1S)-1-(4-((1,1-dimethyl-2,3-dihydro-1H-inden-2-yl)amino)phenyl)-2,2,2-trifluoroethyl)-N-methyltetrahydro-2H-pyran-4-carboxamide;
N-((1S)-1-(4-((1,1-dimethyl-2,3-dihydro-1H-inden-2-yl)amino)phenyl)-2,2,2-trifluoroethyl)-N-methyl-2-oxo-1,2-dihydropyridine-4-carboxamide;
N-((1S)-1-(4-((1,1-dimethyl-2,3-dihydro-1H-inden-2-yl)amino)phenyl)-2,2,2-trifluoroethyl)-4-hydroxy-N-methylpiperidine-1-carboxamide;
3-acetamido-N-((1S)-1-(4-((1,1-dimethyl-2,3-dihydro-1H-inden-2-yl)amino)phenyl)-2,2,2-trifluoroethyl)-N-methylpropanamide;
N-((1S)-1-(4-((1,1-dimethyl-2,3-dihydro-1H-inden-2-yl)amino)phenyl)-2,2,2-trifluoroethyl)-N-methyloxazole-4-carboxamide;
N-((1S)-1-(4-((1,1-dimethyl-2,3-dihydro-1H-inden-2-yl)amino)phenyl)-2,2,2-trifluoroethyl)-2-methoxy-N-methylacetamide;
N-((1S)-1-(4-((1,1-dimethyl-2,3-dihydro-1H-inden-2-yl)amino)phenyl)-2,2,2-trifluoroethyl)-2-hydroxy-N-methylacetamide;
N-((1S)-1-(4-((1,1-dimethyl-2,3-dihydro-1H-inden-2-yl)amino)phenyl)-2,2,2-trifluoroethyl)-3-methoxy-N-methylpropanamide;
N-((1S)-1-(4-((1,1-dimethyl-2,3-dihydro-1H-inden-2-yl)amino)phenyl)-2,2,2-trifluoroethyl)-3-hydroxy-N-methylpropanamide;
N-((1S)-1-(6-((1,1-dimethyl-2,3-dihydro-1H-inden-2-yl)amino)pyridin-3-yl)-2,2,2-trifluoroethyl)-N-methyltetrahydro-2H-thiopyran-4-carboxamide 1,1-dioxide;
N-((1S)-1-(4-((1,3-dimethyl-2,3-dihydro-1H-inden-2-yl)amino)phenyl)-2,2,2-trifluoroethyl)-N-methyltetrahydro-2H-thiopyran-4-carboxamide 1,1-dioxide;
2-acetamido-N-((1S)-1-(4-((1,1-dimethyl-2,3-dihydro-1H-inden-2-yl)amino)phenyl)-2,2,2-trifluoroethyl)-N-methylacetamide;
1-acetyl-N-((1S)-1-(4-((1,1-dimethyl-2,3-dihydro-1H-inden-2-yl)amino)phenyl)-2,2,2-trifluoroethyl)-N,3-dimethylazetidine-3-carboxamide;
1-acetyl-N-((1S)-1-(4-((1,1-dimethyl-2,3-dihydro-1H-inden-2-yl)amino)phenyl)-2,2,2-trifluoroethyl)-3-fluoro-N-methylazetidine-3-carboxamide;
N-((1S)-1-(4-((1,1-dimethyl-2,3-dihydro-1H-inden-2-yl)amino)phenyl)-2,2,2-trifluoroethyl)-N-methyl-2-(2-oxopyrrolidin-1-yl)acetamide;
N-((1S)-1-(4-((1,1-dimethyl-2,3-dihydro-1H-inden-2-yl)amino)phenyl)-2,2,2-trifluoroethyl)-N-methyl-3-(2-oxopyrrolidin-1-yl)propenamide;
5-(((1S)-1-(4-((1,1-dimethyl-2,3-dihydro-1H-inden-2-yl)amino)phenyl)-2,2,2-trifluoroethyl)(methyl)amino)-5-oxopentanoic acid;
N-((1S)-1-(4-((1,1-dimethyl-2,3-dihydro-1H-inden-2-yl)amino)phenyl)-2,2,2-trifluoroethyl)-N-methyl-1,2,5-oxadiazole-3-carboxamide;
4-(((1S)-1-(4-((1,1-dimethyl-2,3-dihydro-1H-inden-2-yl)amino)phenyl)-2,2,2-trifluoroethyl)(methyl)amino)-4-oxobutanoic acid;
N-methyl-N-((1S)-2,2,2-trifluoro-1-(4-((5-methoxy-2,3-dihydro-1H-inden-2-yl)amino)phenyl)ethyl)tetrahydro-2H-thiopyran-4-carboxamide 1,1-dioxide;
N-((1S)-1-(4-((5-chloro-2,3-dihydro-1H-inden-2-yl)amino)phenyl)-2,2,2-trifluoroethyl)-N-methyltetrahydro-2H-thiopyran-4-carboxamide 1,1-dioxide;
1-acetylazetidin-3-yl ((1S)-1-(4-((1,1-dimethyl-2,3-dihydro-1H-inden-2-yl)amino)phenyl)-2,2,2-trifluoroethyl)(methyl)carbamate;
tert-butyl 3-((((1S)-1-(4-((1,1-dimethyl-2,3-dihydro-1H-inden-2-yl)amino)phenyl)-2,2,2-trifluoroethyl)(methyl)carbamoyl)oxy)azetidine-1-carboxylate;
azetidin-3-yl ((1S)-1-(4-((1,1-dimethyl-2,3-dihydro-1H-inden-2-yl)amino)phenyl)-2,2,2-trifluoroethyl)(methyl)carbamate hydrochloride;
N-((1S)-1-(4-((1,1-dimethyl-2,3-dihydro-1H-inden-2-yl)amino)phenyl)-2,2,2-trifluoroethyl)-N-methyl-3-(N-methylsulfamoyl)propenamide;
N-((1R)-1-(4-((1,1-dimethyl-2,3-dihydro-1H-inden-2-yl)amino)phenyl)ethyl)-N-methyltetrahydro-2H-thiopyran-4-carboxamide 1,1-dioxide;
N-((1S)-1-(4-((1,1-dimethyl-2,3-dihydro-1H-inden-2-yl)amino)phenyl)-2,2,2-trifluoroethyl)-N-methyl-4-(N-methylsulfamoyl)butanamide;
N-((1S)-1-(4-((1,1-dimethyl-2,3-dihydro-1H-inden-2-yl)amino)phenyl)-2,2,2-trifluoroethyl)-2-(2,4-dioxoimidazolidin-1-yl)-N-methylacetamide;
N-((1S)-1-(4-((1,1-dimethyl-2,3-dihydro-1H-inden-2-yl)amino)phenyl)-2,2,2-trifluoroethyl)-N-methyl-1-(methylsulfonyl)azetidine-3-carboxamide;
(S)—N-(1-(4-((2,3-dihydro-1H-inden-2-yl)amino)phenyl)-2,2,2-trifluoroethyl)-N-methylpiperidine-4-carboxamide;
(S)—N-(1-(5-((2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(4-((1,1-dimethyl-2,3-dihydro-1H-inden-2-yl)amino)phenyl)-2,2,2-trifluoroethyl)-N-methylazetidine-3-carboxamide;
N3-((1S)-1-(4-((1,1-dimethyl-2,3-dihydro-1H-inden-2-yl)amino)phenyl)-2,2,2-trifluoroethyl)-N1,N3-dimethylazetidine-1,3-dicarboxamide;
N-((1S)-1-(4-((1,1-dimethyl-2,3-dihydro-1H-inden-2-yl)amino)phenyl)-2,2,2-trifluoroethyl)-N,1-dimethylazetidine-3-carboxamide;
N-((1S)-1-(5-((1,1-dimethyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(4-((1,1-dimethyl-2,3-dihydro-1H-inden-2-yl)amino)phenyl)-2,2,2-trifluoroethyl)-N-methylcyclopropanesulfonamide;
N-((1S)-1-(4-((1,1-dimethyl-2,3-dihydro-1H-inden-2-yl)amino)phenyl)-2,2,2-trifluoroethyl)tetrahydro-2H-thiopyran-4-carboxamide 1,1-dioxide;
N-(1-(5-((1,1-dimethyl-2,3-dihydro-1H-inden-2-yl)amino)pyridin-2-yl)-2,2,2-trifluoroethyl)-N-methylazetidine-3-carboxamide;
N-(1-(6-((1,1-dimethyl-2,3-dihydro-1H-inden-2-yl)amino)pyridin-3-yl)-2,2,2-trifluoroethyl)-N-methylazetidine-3-carboxamide;
N-(1-(5-((1,1-dimethyl-2,3-dihydro-1H-inden-2-yl)amino)pyridin-2-yl)-2,2,2-trifluoroethyl)-N,1-dimethylazetidine-3-carboxamide;
N-(1-(6-((1,1-dimethyl-2,3-dihydro-1H-inden-2-yl)amino)pyridin-3-yl)-2,2,2-trifluoroethyl)-N,1-dimethylazetidine-3-carboxamide;
N-(1-(5-((1,1-dimethyl-2,3-dihydro-1H-inden-2-yl)amino)pyridin-2-yl)-2,2,2-trifluoroethyl)-N-methyl-2-oxo-1,2-dihydropyridine-4-carboxamide;
N-(1-(6-((1,1-dimethyl-2,3-dihydro-1H-inden-2-yl)amino)pyridin-3-yl)-2,2,2-trifluoroethyl)-N-methyl-2-oxo-1,2-dihydropyridine-4-carboxamide;
N-((1S)-1-(5-((1,1-dimethyl-2,3-dihydro-1H-inden-2-yl)amino)pyridin-2-yl)-2,2,2-trifluoroethyl)-N-methyl-6-oxopiperidine-3-carboxamide;
N-((1S)-1-(5-((1,1-dimethyl-2,3-dihydro-1H-inden-2-yl)amino)pyridin-2-yl)-2,2,2-trifluoroethyl)-N-methyl-2-oxooxazolidine-5-carboxamide;
N-((1S)-1-(5-((1,1-dimethyl-2,3-dihydro-1H-inden-2-yl)amino)pyridin-2-yl)-2,2,2-trifluoroethyl)-3-(dimethylamino)-N-methylcyclobutane-1-carboxamide;
N-((1S)-1-(6-((1,1-Dimethyl-2,3-dihydro-1H-inden-2-yl)amino)pyridin-3-yl)-2,2,2-trifluoroethyl)-3-trans-(dimethylamino)-N-methylcyclobutane-1-carboxamide;
N-((1S)-1-(5-((1,1-dimethyl-2,3-dihydro-1H-inden-2-yl)amino)pyridin-2-yl)-2,2,2-trifluoroethyl)-N-methyl-2-oxopiperidine-4-carboxamide;
N-((1S)-1-(6-((1,1-dimethyl-2,3-dihydro-1H-inden-2-yl)amino)pyridin-3-yl)-2,2,2-trifluoroethyl)-N-methylquinuclidine-3-carboxamide;
N-((1S)-1-(5-((1,1-dimethyl-2,3-dihydro-1H-inden-2-yl)amino)pyridin-2-yl)-2,2,2-trifluoroethyl)-N-methylpiperidine-3-carboxamide;
N-((1S)-1-(5-((1,1-dimethyl-2,3-dihydro-1H-inden-2-yl)amino)pyridin-2-yl)-2,2,2-trifluoroethyl)-N,1-dimethylpiperidine-3-carboxamide;
N-((1S)-1-(5-((1,1-dimethyl-2,3-dihydro-1H-inden-2-yl)amino)pyridin-2-yl)-2,2,2-trifluoroethyl)-N,1-dimethylpiperidine-4-carboxamide;
N-((1S)-1-(5-((1,1-dimethyl-2,3-dihydro-1H-inden-2-yl)amino)pyridin-2-yl)-2,2,2-trifluoroethyl)-N-methyl-5-oxomorpholine-2-carboxamide;
N-((1S)-1-(5-((1,1-dimethyl-2,3-dihydro-1H-inden-2-yl)amino)pyridin-2-yl)-2,2,2-trifluoroethyl)-N-methylquinuclidine-3-carboxamide;
N-((1S)-1-(5-((1,1-dimethyl-2,3-dihydro-1H-inden-2-yl)amino)pyridin-2-yl)-2,2,2-trifluoroethyl)-N-methylthiomorpholine-4-carboxamide 1,1-dioxide;
N-((1S)-1-(6-((1,1-dimethyl-2,3-dihydro-1H-inden-2-yl)amino)pyridin-3-yl)-2,2,2-trifluoroethyl)-N,1-dimethylpiperidine-4-carboxamide;
N-((1S)-1-(5-((1,1-dimethyl-2,3-dihydro-1H-inden-2-yl)amino)pyridin-2-yl)-2,2,2-trifluoroethyl)-N-methylpiperidine-4-carboxamide;
N-((1S)-1-(6-((1,1-dimethyl-2,3-dihydro-1H-inden-2-yl)amino)pyridin-3-yl)-2,2,2-trifluoroethyl)-N-methylpiperidine-4-carboxamide;
N-((1S)-1-(4-((6-chloro-1,1-dimethyl-2,3-dihydro-1H-inden-2-yl)amino)phenyl)-2,2,2-trifluoroethyl)-N-methyltetrahydro-2H-thiopyran-4-carboxamide 1,1-dioxide;
N-((1S)-1-(4-((5-chloro-1,1-dimethyl-2,3-dihydro-1H-inden-2-yl)amino)phenyl)-2,2,2-trifluoroethyl)-N-methyltetrahydro-2H-thiopyran-4-carboxamide 1,1-dioxide;
N-((1S)-1-(5-((1,1-dimethyl-2,3-dihydro-1H-inden-2-yl)amino)pyridin-2-yl)-2,2,2-trifluoroethyl)-N-methyl-1-azaspiro[3.3]heptane-6-carboxamide;
(4,6-trans)-N-((1S)-1-(5-((1,1-dimethyl-2,3-dihydro-1H-inden-2-yl)amino)pyridin-2-yl)-2,2,2-trifluoroethyl)-N,1-dimethyl-1-azaspiro[3.3]heptane-6-carboxamide;
(4,6-cis)-N-((1S)-1-(5-((1,1-dimethyl-2,3-dihydro-1H-inden-2-yl)amino)pyridin-2-yl)-2,2,2-trifluoroethyl)-N,1-dimethyl-1-azaspiro[3.3]heptane-6-carboxamide;
(S)—N—((S)-1-(5-(((S)-1,1-dimethyl-2,3-dihydro-1H-inden-2-yl)amino)pyridin-2-yl)-2,2,2-trifluoroethyl)-N-methyl-5-oxopyrrolidine-3-carboxamide;
N-((1S)-1-(5-((1,1-dimethyl-2,3-dihydro-1H-inden-2-yl)amino)pyridin-2-yl)-2,2,2-trifluoroethyl)-N-methyl-5-oxomorpholine-3-carboxamide;
(S)—N—((S)-1-(5-(((S)-1,1-dimethyl-2,3-dihydro-1H-inden-2-yl)amino)pyridin-2-yl)-2,2,2-trifluoroethyl)-N-methyl-4-oxoazetidine-2-carboxamide;
(R)—N—((S)-1-(5-(((S)-1,1-dimethyl-2,3-dihydro-1H-inden-2-yl)amino)pyridin-2-yl)-2,2,2-trifluoroethyl)-N-methyl-5-oxopyrrolidine-3-carboxamide;
(S)—N—((S)-1-(5-(((S)-1,1-dimethyl-2,3-dihydro-1H-inden-2-yl)amino)pyridin-2-yl)-2,2,2-trifluoroethyl)-N-methylmorpholine-2-carboxamide dihydrochloride;
(S)—N—((S)-1-(5-(((S)-1,1-dimethyl-2,3-dihydro-1H-inden-2-yl)amino)pyridin-2-yl)-2,2,2-trifluoroethyl)-N-methylpyrrolidine-3-carboxamide;
(S)-1-acetyl-N—((S)-1-(5-(((S)-1,1-dimethyl-2,3-dihydro-1H-inden-2-yl)amino)pyridin-2-yl)-2,2,2-trifluoroethyl)-N-methylpyrrolidine-3-carboxamide;
(S)—N—((S)-1-(5-(((S)-1,1-dimethyl-2,3-dihydro-1H-inden-2-yl)amino)pyridin-2-yl)-2,2,2-trifluoroethyl)-N-methyl-5-oxopyrrolidine-2-carboxamide;
(R)-1-acetyl-N—((S)-1-(5-(((S)-1,1-dimethyl-2,3-dihydro-1H-inden-2-yl)amino)pyridin-2-yl)-2,2,2-trifluoroethyl)-N-methylpyrrolidine-2-carboxamide;
(1R,3S)-3-acetamido-N—((S)-1-(5-(((S)-1,1-dimethyl-2,3-dihydro-1H-inden-2-yl)amino)pyridin-2-yl)-2,2,2-trifluoroethyl)-N-methylcyclopentane-1-carboxamide;
N—((S)-1-(5-(((S)-1,1-dimethyl-2,3-dihydro-1H-inden-2-yl)amino)pyridin-2-yl)-2,2,2-trifluoroethyl)-N-methyl-2-azaspiro[3.3]heptane-6-carboxamide;
N-((1S)-1-(6-((1,1-dimethyl-2,3-dihydro-1H-inden-2-yl)amino)pyridin-3-yl)-2,2,2-trifluoroethyl)-N-methyl-7-oxo-6-oxa-8-azaspiro[3.5]nonane-2-carboxamide;
(R)—N—((S)-1-(5-(((S)-1,1-dimethyl-2,3-dihydro-1H-inden-2-yl)amino)pyridin-2-yl)-2,2,2-trifluoroethyl)-N-methylpyrrolidine-3-carboxamide;
N-methyl-N-((1S)-2,2,2-trifluoro-1-(5-((4-methoxy-2,3-dihydro-1H-inden-2-yl)amino)pyridin-2-yl)ethyl)tetrahydro-2H-thiopyran-4-carboxamide 1,1-dioxide;
3-acetamido-N—((S)-1-(5-(((S)-1,1-dimethyl-2,3-dihydro-1H-inden-2-yl)amino)pyridin-2-yl)-2,2,2-trifluoroethyl)-N-methylbicyclo[1.1.1]pentane-1-carboxamide;
(R)—N—((S)-1-(5-(((S)-1,1-dimethyl-2,3-dihydro-1H-inden-2-yl)amino)pyridin-2-yl)-2,2,2-trifluoroethyl)-N,4-dimethylmorpholine-2-carboxamide;
(S)-4-acetyl-N—((S)-1-(5-(((S)-1,1-dimethyl-2,3-dihydro-1H-inden-2-yl)amino)pyridin-2-yl)-2,2,2-trifluoroethyl)-N-methylmorpholine-2-carboxamide;
(1,3-cis)-3-acetamido-N—((S)-1-(5-(((S)-1,1-dimethyl-2,3-dihydro-1H-inden-2-yl)amino)pyridin-2-yl)-2,2,2-trifluoroethyl)-N-methylcyclobutane-1-carboxamide;
(2S,4R)-1-acetyl-N—((S)-1-(5-(((S)-1,1-dimethyl-2,3-dihydro-1H-inden-2-yl)amino)pyridin-2-yl)-2,2,2-trifluoroethyl)-4-hydroxy-N-methylpyrrolidine-2-carboxamide;
(1R,3S)—N—((S)-1-(5-(((S)-1,1-dimethyl-2,3-dihydro-1H-inden-2-yl)amino)pyridin-2-yl)-2,2,2-trifluoroethyl)-N-methyl-3-(2,2,2-trifluoroacetamido)cyclopentane-1-carboxamide;
N1-((S)-1-(5-(((S)-1,1-dimethyl-2,3-dihydro-1H-inden-2-yl)amino)pyridin-2-yl)-2,2,2-trifluoroethyl)-N1,N3-dimethylbicyclo[1.1.1]pentane-1,3-dicarboxamide;
(1,3-trans)-3-acetamido-N—((S)-1-(5-(((S)-1,1-dimethyl-2,3-dihydro-1H-inden-2-yl)amino)pyridin-2-yl)-2,2,2-trifluoroethyl)-N-methylcyclobutane-1-carboxamide;
(1,3-cis)-3-acetamido-N—((S)-1-(5-(((S)-1,1-dimethyl-2,3-dihydro-1H-inden-2-yl)amino)pyridin-2-yl)-2,2,2-trifluoroethyl)-N-methylcyclopentane-1-carboxamide;
N-((1S)-1-(5-((1,1-dimethyl-2,3-dihydro-1H-inden-2-yl)amino)pyridin-2-yl)-2,2,2-trifluoroethyl)-N-ethyltetrahydro-2H-thiopyran-4-carboxamide 1,1-dioxide;
N-methyl-N-((1S)-2,2,2-trifluoro-1-(5-((4-(trifluoromethyl)-2,3-dihydro-1H-inden-2-yl)amino)pyridin-2-yl)ethyl)tetrahydro-2H-thiopyran-4-carboxamide 1,1-dioxide;
(R)-1-acetyl-N—((S)-1-(5-(((S)-1,1-dimethyl-2,3-dihydro-1H-inden-2-yl)amino)pyridin-2-yl)-2,2,2-trifluoroethyl)-N-methylpyrrolidine-3-carboxamide;
(R)—N—((S)-1-(5-(((S)-1,1-dimethyl-2,3-dihydro-1H-inden-2-yl)amino)pyridin-2-yl)-2,2,2-trifluoroethyl)-N-methylmorpholine-2-carboxamide;
(S)-1-acetyl-N—((S)-1-(5-(((S)-1,1-dimethyl-2,3-dihydro-1H-inden-2-yl)amino)pyridin-2-yl)-2,2,2-trifluoroethyl)-N-methylpyrrolidine-2-carboxamide;
(S)—N—((S)-1-(5-(((S)-1,1-dimethyl-2,3-dihydro-1H-inden-2-yl)amino)pyridin-2-yl)-2,2,2-trifluoroethyl)-N,4-dimethylmorpholine-2-carboxamide;
N—((S)-1-(5-(((S)-1,1-dimethyl-2,3-dihydro-1H-inden-2-yl)amino)pyridin-2-yl)-2,2,2-trifluoroethyl)-2-(1,1-dioxidotetrahydro-2H-thiopyran-4-yl)-N-methylacetamide;
N-((1S)-1-(5-((4-ethyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N—((S)-1-(5-(((S)-5-chloro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N—((S)-1-(5-(((R)-5-chloro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N—((S)-1-(5-(((S)-1,1-dimethyl-2,3-dihydro-1H-inden-2-yl)amino)pyridin-2-yl)-2,2,2-trifluoroethyl)-N,1-dimethyl-5-oxopiperazine-2-carboxamide;
N—((R)-1-(5-(((R)-1,1-dimethyl-2,3-dihydro-1H-inden-2-yl)amino)pyridin-2-yl)-2,2,2-trifluoroethyl)-N,1-dimethyl-5-oxopiperazine-2-carboxamide;
N1-((S)-1-(5-(((R)-1,1-dimethyl-2,3-dihydro-1H-inden-2-yl)amino)pyridin-2-yl)-2,2,2-trifluoroethyl)-N1,N3-dimethylbicyclo[1.1.1]pentane-1,3-dicarboxamide;
(1R,3S)-3-acetamido-N—((S)-1-(5-(((R)-1,1-dimethyl-2,3-dihydro-1H-inden-2-yl)amino)pyridin-2-yl)-2,2,2-trifluoroethyl)-N-methylcyclopentane-1-carboxamide;
(1,3-cis)-3-acetamido-N—((S)-1-(5-(((R)-1,1-dimethyl-2,3-dihydro-1H-inden-2-yl)amino)pyridin-2-yl)-2,2,2-trifluoroethyl)-N-methylcyclobutane-1-carboxamide;
(1,3-trans)-3-acetamido-N—((S)-1-(5-(((R)-1,1-dimethyl-2,3-dihydro-1H-inden-2-yl)amino)pyridin-2-yl)-2,2,2-trifluoroethyl)-N-methylcyclobutane-1-carboxamide;
(2,4-cis)-N-((1S)-1-(5-((1,1-dimethyl-2,3-dihydro-1H-inden-2-yl)amino)pyridin-2-yl)-2,2,2-trifluoroethyl)-N-methyl-6-oxo-7-oxa-5-azaspiro[3.4]octane-2-carboxamide;
N-((1S)-1-(5-((1,1-dimethyl-2,3-dihydro-1H-inden-2-yl)amino)pyridin-2-yl)-2,2,2-trifluoroethyl)-N-methyl-1-(methylsulfonyl)azetidine-3-carboxamide;
(S)—N—((S)-1-(5-(((R)-1,1-dimethyl-2,3-dihydro-1H-inden-2-yl)amino)pyridin-2-yl)-2,2,2-trifluoroethyl)-N-methyl-5-oxopyrrolidine-3-carboxamide;
N-((1S)-1-(5-((1,1-dimethyl-2,3-dihydro-1H-inden-2-yl)amino)pyridin-2-yl)-2,2,2-trifluoroethyl)-2-(2,4-dioxoimidazolidin-1-yl)-N-methylacetamide;
N-((1S)-1-(5-((1,1-dimethyl-2,3-dihydro-1H-inden-2-yl)amino)pyridin-2-yl)-2,2,2-trifluoroethyl)-N-methyl-3-thiabicyclo[3.1.0]hexane-6-carboxamide 3,3-dioxide;
(R)—N—((S)-1-(6-(((S)-1,1-dimethyl-2,3-dihydro-1H-inden-2-yl)amino)pyridin-3-yl)-2,2,2-trifluoroethyl)-N-methyl-6-oxopiperidine-3-carboxamide;
(S)—N—((S)-1-(6-(((S)-1,1-dimethyl-2,3-dihydro-1H-inden-2-yl)amino)pyridin-3-yl)-2,2,2-trifluoroethyl)-N-methyl-6-oxopiperidine-3-carboxamide;
(2S,4R)-1-acetyl-N—((S)-1-(5-(((R)-1,1-dimethyl-2,3-dihydro-1H-inden-2-yl)amino)pyridin-2-yl)-2,2,2-trifluoroethyl)-4-hydroxy-N-methylpyrrolidine-2-carboxamide;
(1S, 3R)—N—((S)-1-(5-(((S)-1,1-dimethyl-2,3-dihydro-1H-inden-2-yl)amino)pyridin-2-yl)-2,2,2-trifluoroethyl)-N-methyl-3-(methylsulfonamido)cyclopentane-1-carboxamide;
(1,3-trans)-N—((S)-1-(5-(((S)-1,1-dimethyl-2,3-dihydro-1H-inden-2-yl)amino)pyridin-2-yl)-2,2,2-trifluoroethyl)-N-methyl-3-(methylsulfonamido)cyclobutane-1-carboxamide;
N—((S)-1-(5-(((S)-1,1-dimethyl-2,3-dihydro-1H-inden-2-yl)amino)pyridin-2-yl)-2,2,2-trifluoroethyl)-N-methyl-1-(methylsulfonyl)piperidine-4-carboxamide;
1-acetyl-N—((S)-1-(5-(((S)-1,1-dimethyl-2,3-dihydro-1H-inden-2-yl)amino)pyridin-2-yl)-2,2,2-trifluoroethyl)-N-methylpiperidine-4-carboxamide;
N—((S)-1-(5-(((S)-1,1-dimethyl-2,3-dihydro-1H-inden-2-yl)amino)pyridin-2-yl)-2,2,2-trifluoroethyl)-N,1-dimethyl-5-oxopyrrolidine-2-carboxamide;
N³-((1S)-1-(5-((1,1-dimethyl-2,3-dihydro-1H-inden-2-yl)amino)pyridin-2-yl)-2,2,2-trifluoroethyl)-N¹,N³-dimethylazetidine-1,3-dicarboxamide;
N-((1S)-1-(5-((1,1-dimethyl-2,3-dihydro-1H-inden-2-yl)amino)pyridin-2-yl)-2,2,2-trifluoroethyl)-N-methyl-6-oxo-5-azaspiro[3.4]octane-2-carboxamide;
N—((S)-1-(5-(((S)-1,1-dimethyl-2,3-dihydro-1H-inden-2-yl)amino)pyridin-2-yl)-2,2,2-trifluoroethyl)-N-methyl-3-(methylsulfonamido)bicyclo[1.1.1]pentane-1-carboxamide;
N—((S)-1-(5-(((S)-1,1-dimethyl-2,3-dihydro-1H-inden-2-yl)amino)pyridin-2-yl)-2,2,2-trifluoroethyl)-2-(1,1-dioxidothiomorpholino)-N-methylacetamide;
1-acetyl-N-((1S)-1-(5-((1,1-dimethyl-2,3-dihydro-1H-inden-2-yl)amino)pyridin-2-yl)-2,2,2-trifluoroethyl)-N-methyl-1-azaspiro[3.3]heptane-6-carboxamide;
N-((1S)-1-(5-((4-bromo-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N⁶-((1S)-1-(5-((1,1-dimethyl-2,3-dihydro-1H-inden-2-yl)amino)pyridin-2-yl)-2,2,2-trifluoroethyl)-N¹,N⁶-dimethyl-1-azaspiro[3.3]heptane-1,6-dicarboxamide;
(1,3-cis)-N¹—((S)-1-(5-(((S)-1,1-dimethyl-2,3-dihydro-1H-inden-2-yl)amino)pyridin-2-yl)-2,2,2-trifluoroethyl)-N¹,N³-dimethylcyclobutane-1,3-dicarboxamide;
(1,3-cis)-N—((S)-1-(5-(((S)-1,1-dimethyl-2,3-dihydro-1H-inden-2-yl)amino)pyridin-2-yl)-2,2,2-trifluoroethyl)-N-methyl-3-(methylsulfonamido)cyclobutane-1-carboxamide;
N-((1S)-1-(5-((1,1-dimethyl-2,3-dihydro-1H-inden-2-yl)amino)pyridin-2-yl)-2,2,2-trifluoroethyl)-1-(2-methoxyethyl)-N-methyl-1-azaspiro[3.3]heptane-6-carboxamide;
(2S,4R)—N—((S)-1-(5-(((S)-1,1-dimethyl-2,3-dihydro-1H-inden-2-yl)amino)pyridin-2-yl)-2,2,2-trifluoroethyl)-4-hydroxy-N-methyl-1-(methylsulfonyl)pyrrolidine-2-carboxamide;
N-((1S)-1-(5-((1,1-dimethyl-2,3-dihydro-1H-inden-2-yl)amino)pyridin-2-yl)-2,2,2-trifluoroethyl)-N-methyl-2-(2-oxoimidazolidin-1-yl)acetamide;
(1,3-trans)-N¹—((S)-1-(5-(((S)-1,1-dimethyl-2,3-dihydro-1H-inden-2-yl)amino)pyridin-2-yl)-2,2,2-trifluoroethyl)-N¹,N³-dimethylcyclobutane-1,3-dicarboxamide;
N-((1S)-1-(5-((1,1-dimethyl-2,3-dihydro-1H-inden-2-yl)amino)pyridin-2-yl)-2,2,2-trifluoroethyl)-1-(2-hydroxyethyl)-N-methyl-1-azaspiro[3.3]heptane-6-carboxamide;
N—((S)-1-(5-(((S)-1,1-dimethyl-2,3-dihydro-1H-inden-2-yl)amino)pyridin-2-yl)-2,2,2-trifluoroethyl)-N,1-dimethyl-2-oxopiperidine-4-carboxamide;
N—((S)-1-(5-(((S)-1,1-dimethyl-2,3-dihydro-1H-inden-2-yl)amino)pyridin-2-yl)-2,2,2-trifluoroethyl)-N,1-dimethyl-6-oxopiperidine-3-carboxamide;
N-((1S)-1-(5-((4-cyclopropyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-methyl-N-((1S)-2,2,2-trifluoro-1-(5-((5-(trifluoromethyl)-2,3-dihydro-1H-inden-2-yl)amino)pyridin-2-yl)ethyl)tetrahydro-2H-thiopyran-4-carboxamide 1,1-dioxide;
N-((1S)-1-(5-((4-cyano-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-methyl-N-((1S)-2,2,2-trifluoro-1-(5-((5-fluoro-2,3-dihydro-1H-inden-2-yl)amino)pyridin-2-yl)ethyl)tetrahydro-2H-thiopyran-4-carboxamide 1,1-dioxide;
(S)—N-(1-(5-((5,6-dimethyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4,5-difluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4-chloro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4-chloro-5-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
(S)—N-(1-(5-((4,7-dimethyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4,6-difluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((6-chloro-4-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
(S)—N-(1-(5-((5,6-difluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
(S)—N-(1-(5-((4,7-difluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-methyl-N-((1S)-2,2,2-trifluoro-1-(5-((4-fluoro-2,3-dihydro-1H-inden-2-yl)amino)pyridin-2-yl)ethyl)tetrahydro-2H-thiopyran-4-carboxamide 1,1-dioxide;
N-((1S)-1-(5-((5-chloro-4-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4,5-dichloro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-methyl-N-((1S)-2,2,2-trifluoro-1-(6-((4-(trifluoromethyl)-2,3-dihydro-1H-inden-2-yl)amino)pyridin-3-yl)ethyl)tetrahydro-2H-thiopyran-4-carboxamide 1,1-dioxide;
N-((1S)-1-(5-((4,6-dichloro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((5-bromo-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4-chloro-6-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(6-((5-chloro-2,3-dihydro-1H-inden-2-yl)amino)pyridin-3-yl)-2,2,2-trifluoroethyl)-N-methyltetrahydro-2H-thiopyran-4-carboxamide 1,1-dioxide;
N-((1S)-1-(5-((5-chloro-4-fluoro-2,3-dihydro-1H-inden-2-yl)amino)pyridin-2-yl)-2,2,2-trifluoroethyl)-N-methyl-1-(methylsulfonamido)cyclopropane-1-carboxamide;
N-((1S)-1-(5-((5-chloro-6-(difluoromethoxy)-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N¹-((1S)-1-(5-((5-chloro-2,3-dihydro-1H-inden-2-yl)amino)pyridin-2-yl)-2,2,2-trifluoroethyl)-N¹,N⁴-dimethylcubane-1,4-dicarboxamide;
N-((1S)-1-(5-((4-chloro-7-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-methyl-N-((1S)-2,2,2-trifluoro-1-(5-((4,5,6-trifluoro-2,3-dihydro-1H-inden-2-yl)amino)pyridin-2-yl)ethyl)tetrahydro-2H-thiopyran-4-carboxamide 1,1-dioxide;
(S)—N—((S)-1-(5-(((S)-5-chloro-2,3-dihydro-1H-inden-2-yl)amino)pyridin-2-yl)-2,2,2-trifluoroethyl)-N-methyl-6-oxopiperidine-3-carboxamide;
(S)—N-(1-(5-((5,6-dichloro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N—((S)-1-(5-(((S)-5-chloro-2,3-dihydro-1H-inden-2-yl)amino)pyridin-2-yl)-2,2,2-trifluoroethyl)-N-methyl-3-(methylsulfonamido)bicyclo[1.1.1]pentane-1-carboxamide;
(S)—N—((S)-1-(5-(((S)-5-chloro-2,3-dihydro-1H-inden-2-yl)amino)pyridin-2-yl)-2,2,2-trifluoroethyl)-N-methyl-5-oxopyrrolidine-3-carboxamide;
(R)—N—((S)-1-(5-(((S)-5-chloro-2,3-dihydro-1H-inden-2-yl)amino)pyridin-2-yl)-2,2,2-trifluoroethyl)-N,3-dimethyl-2-oxoimidazolidine-4-carboxamide;
(S)—N-(1-(5-((4,7-dichloro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N—((S)-1-(5-(((S)-5-chloro-2,3-dihydro-1H-inden-2-yl)amino)pyridin-2-yl)-2,2,2-trifluoroethyl)-N-methyl-2-oxooxazolidine-5-carboxamide;
(R)—N—((S)-1-(5-(((S)-5-chloro-2,3-dihydro-1H-inden-2-yl)amino)pyridin-2-yl)-2,2,2-trifluoroethyl)-N-methyl-5-oxopyrrolidine-2-carboxamide hydrochloride;
N-((1S)-1-(5-((5-chloro-4-cyclopropyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((5-cyclopropyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
(R)—N—((S)-1-(5-(((S)-5-chloro-2,3-dihydro-1H-inden-2-yl)amino)pyridin-2-yl)-2,2,2-trifluoroethyl)-N-methyl-6-oxopiperidine-2-carboxamide;
N-((1S)-1-(5-((5-bromo-6-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((5-chloro-4-(trifluoromethyl)-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N—((S)-1-(5-(((S)-5-chloro-2,3-dihydro-1H-inden-2-yl)amino)pyridin-2-yl)-2,2,2-trifluoroethyl)-N-methyl-2-oxopiperidine-4-carboxamide;
(S)—N—((S)-1-(5-(((S)-5-chloro-2,3-dihydro-1H-inden-2-yl)amino)pyridin-2-yl)-2,2,2-trifluoroethyl)-N-methyl-5-oxopyrrolidine-2-carboxamide;
(3S)—N-((1S)-1-(5-((5-chloro-4-fluoro-2,3-dihydro-1H-inden-2-yl)amino)pyridin-2-yl)-2,2,2-trifluoroethyl)-N-methyl-5-oxopyrrolidine-3-carboxamide;
(3S)—N-((1S)-1-(5-((4,5-dichloro-2,3-dihydro-1H-inden-2-yl)amino)pyridin-2-yl)-2,2,2-trifluoroethyl)-N-methyl-5-oxopyrrolidine-3-carboxamide;
(3S)—N-((1S)-1-(5-((4,5-dichloro-2,3-dihydro-1H-inden-2-yl)amino)pyridin-2-yl)-2,2,2-trifluoroethyl)-N-methyl-6-oxopiperidine-3-carboxamide;
N-((1S)-1-(5-((4,5-dichloro-2,3-dihydro-1H-inden-2-yl)amino)pyridin-2-yl)-2,2,2-trifluoroethyl)-N-methyl-3-(methylsulfonamido)bicyclo[1.1.1]pentane-1-carboxamide;
N-((1S)-1-(5-((5-(difluoromethoxy)-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4,5-dichloro-2,3-dihydro-1H-inden-2-yl)amino)pyridin-2-yl)-2,2,2-trifluoroethyl)-N-methyl-1-(methylsulfonyl)piperidine-4-carboxamide;
(3S)—N-((1S)-1-(5-((5-chloro-4-cyclopropyl-2,3-dihydro-1H-inden-2-yl)amino)pyridin-2-yl)-2,2,2-trifluoroethyl)-N-methyl-5-oxopyrrolidine-3-carboxamide;
(3S)—N-((1S)-1-(5-((5-chloro-4-cyclopropyl-2,3-dihydro-1H-inden-2-yl)amino)pyridin-2-yl)-2,2,2-trifluoroethyl)-N-methyl-6-oxopiperidine-3-carboxamide;
N-((1S)-1-(6-((5-chloro-4-fluoro-2,3-dihydro-1H-inden-2-yl)amino)pyridin-3-yl)-2,2,2-trifluoroethyl)-N-methyltetrahydro-2H-thiopyran-4-carboxamide 1,1-dioxide;
N-((1S)-1-(5-((4-(difluoromethoxy)-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N³-((1S)-1-(5-((4,5-dichloro-2,3-dihydro-1H-inden-2-yl)amino)pyridin-2-yl)-2,2,2-trifluoroethyl)-N¹,N³-dimethylazetidine-1,3-dicarboxamide;
(3S)—N-((1S)-1-(5-((5-chloro-4-fluoro-2,3-dihydro-1H-inden-2-yl)amino)pyridin-2-yl)-2,2,2-trifluoroethyl)-N-methyl-6-oxopiperidine-3-carboxamide;
(3R)—N-((1S)-1-(6-((5-chloro-4-fluoro-2,3-dihydro-1H-inden-2-yl)amino)pyridin-3-yl)-2,2,2-trifluoroethyl)-N-methyl-5-oxopyrrolidine-3-carboxamide;
N-((1S)-1-(5-((4,5-dichloro-2,3-dihydro-1H-inden-2-yl)amino)pyridin-2-yl)-2,2,2-trifluoroethyl)-N-methyl-6-oxo-5-azaspiro[3.4]octane-2-carboxamide;
N-((1S)-1-(6-((4,5-dichloro-2,3-dihydro-1H-inden-2-yl)amino)pyridin-3-yl)-2,2,2-trifluoroethyl)-N-methyltetrahydro-2H-thiopyran-4-carboxamide 1,1-dioxide;
(3R)—N-((1S)-1-(6-((4,5-dichloro-2,3-dihydro-1H-inden-2-yl)amino)pyridin-3-yl)-2,2,2-trifluoroethyl)-N-methyl-5-oxopyrrolidine-3-carboxamide;
N-((1S)-1-(5-((5-chloro-4-fluoro-2,3-dihydro-1H-inden-2-yl)amino)pyridin-2-yl)-2,2,2-trifluoroethyl)-N,2-dimethylpyrimidine-5-carboxamide;
2-cyano-N-((1S)-1-(5-((4,5-dichloro-2,3-dihydro-1H-inden-2-yl)amino)pyridin-2-yl)-2,2,2-trifluoroethyl)-N-methylacetamide;
N-((1S)-1-(5-((4,5-dichloro-2,3-dihydro-1H-inden-2-yl)amino)pyridin-2-yl)-2,2,2-trifluoroethyl)-N-methyl-1-(tetrahydrofuran-3-carbonyl)azetidine-3-carboxamide;
N-((1S)-1-(5-((4,5-dichloro-2,3-dihydro-1H-inden-2-yl)amino)pyridin-2-yl)-2,2,2-trifluoroethyl)-N-methylthietane-3-carboxamide 1,1-dioxide;
N-((1S)-1-(5-((5-chloro-4-(methoxymethyl)-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4,5-dichloro-2,3-dihydro-1H-inden-2-yl)amino)pyridin-2-yl)-2,2,2-trifluoroethyl)-N-methyltetrahydrothiophene-3-carboxamide 1,1-dioxide;
N-((1S)-1-(5-((5-chloro-4-fluoro-2,3-dihydro-1H-inden-2-yl)amino)pyridin-2-yl)-2,2,2-trifluoroethyl)-2-(1,1-dioxidoisothiazolidin-2-yl)-N-methylacetamide;
(1,3-trans)-N¹-((1S)-1-(5-((4,5-dichloro-2,3-dihydro-1H-inden-2-yl)amino)pyridin-2-yl)-2,2,2-trifluoroethyl)-N¹,N³-dimethylcyclobutane-1,3-dicarboxamide;
N-((1S)-1-(5-((5-chloro-4-fluoro-2,3-dihydro-1H-inden-2-yl)amino)pyridin-2-yl)-2,2,2-trifluoroethyl)-N-methyl-1-(5-methyl-1,3,4-oxadiazol-2-yl)azetidine-3-carboxamide;
N-((1S)-1-(5-((5-chloro-4-fluoro-2,3-dihydro-1H-inden-2-yl)amino)pyridin-2-yl)-2,2,2-trifluoroethyl)-2-hydroxy-N-methylacetamide;
N-((1S)-1-(5-((5-chloro-4-fluoro-2,3-dihydro-1H-inden-2-yl)amino)pyridin-2-yl)-2,2,2-trifluoroethyl)-N,N′-dimethylcyclopropane-1,1-dicarboxamide;
N-((1S)-1-(5-((5-chloro-4-isopropyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((5-chloro-4-fluoro-2,3-dihydro-1H-inden-2-yl)amino)pyridin-2-yl)-2,2,2-trifluoroethyl)-N-methyl-1-(oxetane-3-carbonyl)azetidine-3-carboxamide;
N-((1S)-1-(5-((5-chloro-4-methyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
(1,3-trans)-N-((1S)-1-(5-((5-chloro-4-fluoro-2,3-dihydro-1H-inden-2-yl)amino)pyridin-2-yl)-2,2,2-trifluoroethyl)-3-hydroxy-N-methylcyclobutane-1-carboxamide;
1-acetyl-N-((1S)-1-(5-((4,5-dichloro-2,3-dihydro-1H-inden-2-yl)amino)pyridin-2-yl)-2,2,2-trifluoroethyl)-N-methylazetidine-3-carboxamide;
N-((1S)-1-(5-((4,5-dichloro-2,3-dihydro-1H-inden-2-yl)amino)pyridin-2-yl)-2,2,2-trifluoroethyl)-1-(2-hydroxyacetyl)-N-methylazetidine-3-carboxamide;
N-((1S)-1-(5-((4,5-dichloro-2,3-dihydro-1H-inden-2-yl)amino)pyridin-2-yl)-2,2,2-trifluoroethyl)-N-methyl-2-(methylsulfonamido)acetamide;
(1,3-cis)-N-((1S)-1-(5-((5-chloro-4-fluoro-2,3-dihydro-1H-inden-2-yl)amino)pyridin-2-yl)-2,2,2-trifluoroethyl)-3-hydroxy-N-methylcyclobutane-1-carboxamide;
N-((1S)-1-(5-((5-chloro-4-fluoro-2,3-dihydro-1H-inden-2-yl)amino)pyridin-2-yl)-2,2,2-trifluoroethyl)-N-methyl-1-(oxetane-2-carbonyl)azetidine-3-carboxamide;
(3S)—N-((1S)-1-(6-((5-chloro-4-fluoro-2,3-dihydro-1H-inden-2-yl)amino)pyridin-3-yl)-2,2,2-trifluoroethyl)-N-methyl-6-oxopiperidine-3-carboxamide;
   or a pharmaceutically acceptable salt thereof.

The various functional groups and substituents making up the compounds of the present invention are typically chosen such that the molecular weight of the compound does not exceed 1000. More usually, the molecular weight of the compound will be less than 750, for example less than 700, or less than 650.

Suitable or preferred features of any compounds of the present invention may also be suitable features of any other aspect.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention typically possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers, diastereoisomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess MALT1 inhibitory activity.

The present invention also encompasses compounds of the invention as defined herein which comprise one or more isotopic substitutions. For example, H may be in any isotopic form, including ¹H, ²H (D) and ³H (T); C may be in any isotopic form including ¹²C, ¹³C, and ¹⁴C; and O may be in any isotopic form, including ¹⁶O and ¹⁸O; and the like.

It is also to be understood that certain compounds of the invention may exist in solvated as well as unsolvated forms such as, for example, hydrated forms. It is to be understood that the invention encompasses all such solvated forms that possess MALT1 inhibitory activity.

It is also to be understood that certain compounds of the invention may exhibit polymorphism, and that the invention encompasses all such forms that possess MALT1 inhibitory activity.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

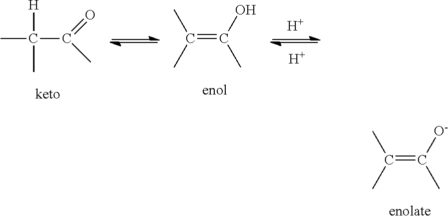

Compounds of the invention containing an amine function may also form N-oxides. A reference herein to a compound of the formula I that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

The compounds of the invention may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached.

Accordingly, the present invention includes those compounds of the formula I as defined hereinbefore when made available by organic synthesis and when made available within the human or animal body by way of cleavage of a pro-drug thereof. Accordingly, the present invention includes those compounds of the formula I that are produced by organic synthetic means and also such compounds that are produced in the human or animal body by way of metabolism of a precursor compound, that is a compound of the formula I may be a synthetically-produced compound or a metabolically-produced compound.

A suitable pharmaceutically acceptable pro-drug of a compound of the formula I is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

The in vivo effects of a compound of the formula I may be exerted in part by one or more metabolites that are formed within the human or animal body after administration of a compound of the formula I. As stated hereinbefore, the in vivo effects of a compound of the formula I may also be exerted by way of metabolism of a precursor compound (a pro-drug).

It shall also be appreciated that compounds of the formula I may also be covalently linked (at any suitable position) to other groups such as, for example, solubilising moieties (for example, PEG polymers), moieties that enable them to be bound to a solid support (such as, for example, biotin-containing moieties), and targeting ligands (such as antibodies or antibody fragments).

### Synthesis

In the description of the synthetic methods described below and in the referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, “Protecting groups in Organic Synthesis (3^{rd} Ed), John Wiley & Sons, NY (1999)”, T. Greene & P. Wuts. Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃·OEt₂. A suitable protecting group for an amino or alkylamino group is, for example, a substituted benzyl group such as 4-methoxybenzyl or 2,4-dimethoxybenzyl. Such a protecting group may be removed by, for example, by treatment with by treatment with a suitable acid as hydrochloric, sulfuric or phosphoric acid or trifluoroacetic acid. A suitable alternative protecting group for a primary amino group is, for example, a phthaloyl group which may be removed by treatment with an alkylamine, for example dimethylaminopropylamine, or with hydrazine.

The person skilled in the art will recognise that the compounds of the invention may be prepared, in known manner, in a variety of ways. Compounds of Formula I can be prepared by the methods given below, by the methods given in the experimental or by analogous methods. The routes described are merely illustrative of some of the methods that can be employed for the synthesis of compounds of Formula I and the person skilled in the art will appreciate that the order of the reaction steps is not limited to those described. It will also be appreciated that the assignment of nucleophile and electrophile is not limited to that described herein and in some cases it may be appropriate for the assignment to be reversed. Different approaches to synthetic chemistry strategy are described in “Organic Synthesis: The Disconnection Approach”, 2^{nd} edition, S. Warren and P. Wyatt (2008).

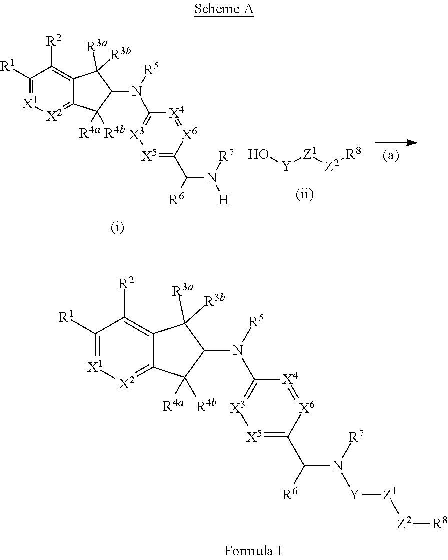

Compounds of Formula I, wherein R¹, R², R^{3a}, R^{3b}, R^{4a}, R^{4b}, R⁵, R⁶, R⁷, x¹, x², X³, X⁴, X⁵, X⁶, Y, Z¹, Z² and R⁸ are as previously defined, can be prepared by the condensation of compounds of Intermediate (i) and Intermediate (ii) in step (a) using a suitable coupling reagent (Scheme A). A suitable coupling reagent includes, for example, propylphosphonic anhydride, phosphorous oxychloride, carbonyldiimidazole or 1-[bis(dimethylamino)-methylene]-1H-1,2,3-triazolo[4,5-b]pyridinium 3-oxide hexafluorophosphate.

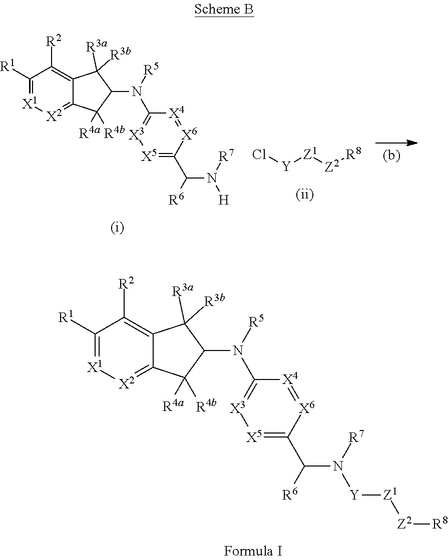

Compounds of Formula I, can also be prepared by the condensation of compounds of Intermediate (i) and Intermediate (iii) in step (b) in the presence of suitable base (Scheme B). A suitable base includes, for example, pyridine, diisopropylethylamine or triethylamine.

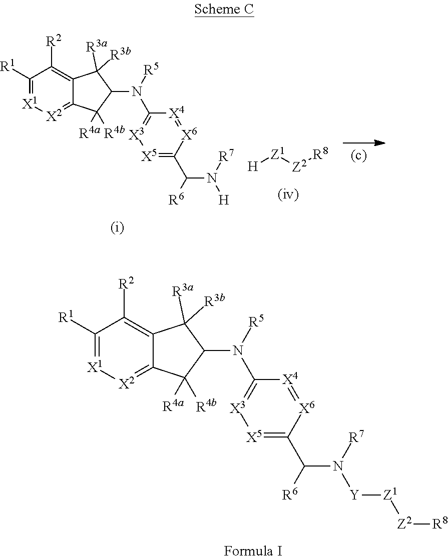

Compounds of Formula I, can also be prepared by the condensation of compounds of Intermediate (i) and Intermediate (iv) in step (c) in the presence of suitable coupling reagent (Scheme C). A suitable coupling reagent includes, for example, phosgene, diphosgene, triphosgene, phenylchloroformate, 4-nitrophenylchloroformate or 1,1-carbonyldiimidazole.

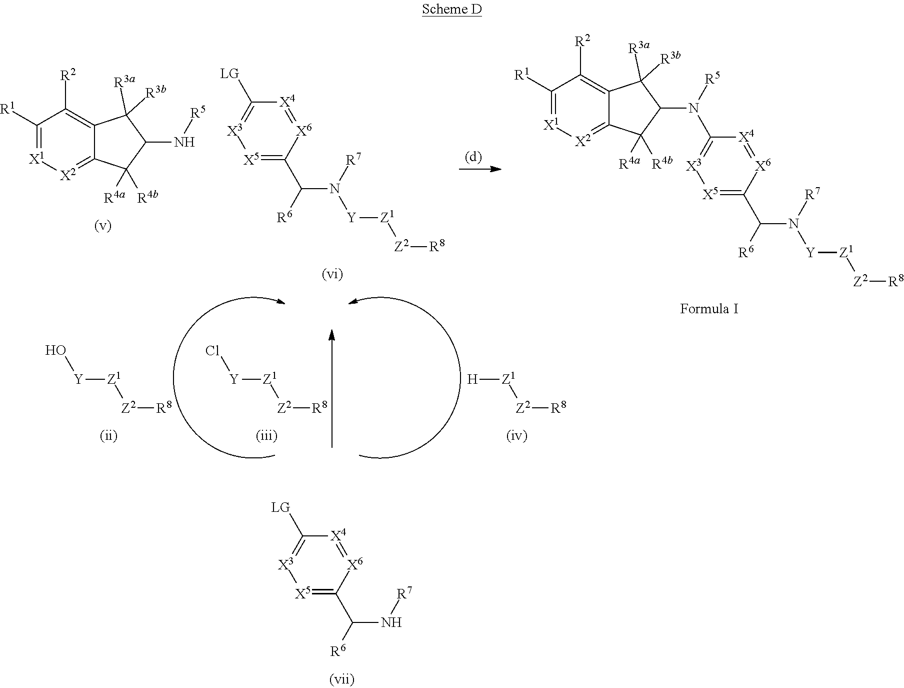

Compounds of Formula I, can also be prepared by the condensation of compounds of Intermediate (v) and Intermediate (vi) in step (d) in the presence of suitable coupling reagent and base (Scheme D). A suitable coupling reagent includes, for example, tBuBrettphosPdG3 in the presence of RuPhos, Pd₂(dba)₃ in the presence of X-Phos, RuPhosPdG3, XPhosPdG2 or BuBrettphosPdG1 in the presence of BrettPhos. A suitable base includes, for example, Cs₂CO₃, K₃CO₃, CsF or sodium t-butoxide. A suitable leaving group (LG) includes, for example, bromide, chloride or triflate.

Intermediate (vi) can be prepared from intermediate (vii) as outlined above with suitable Intermediate (ii), (iii) or (iv) respectively.

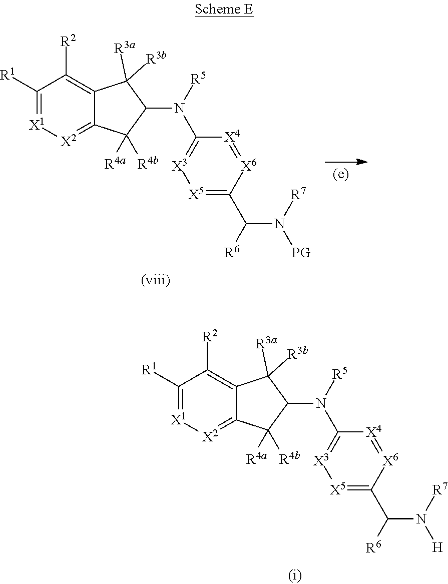

Compounds of Intermediate (i) can be prepared by the removal of a suitable protecting group (PG) (Scheme E). A suitable protecting group includes, for example, includes tert-butoxycarbonyl, pivaloyl or t-butylsulfinyl. The removal of the protecting group may occur, for example, under basic, acidic or reductive conditions using methods as described in “Protecting groups in Organic Synthesis (3^{rd} Ed), John Wiley & Sons, NY (1999)”, T. Greene & P. Wuts.

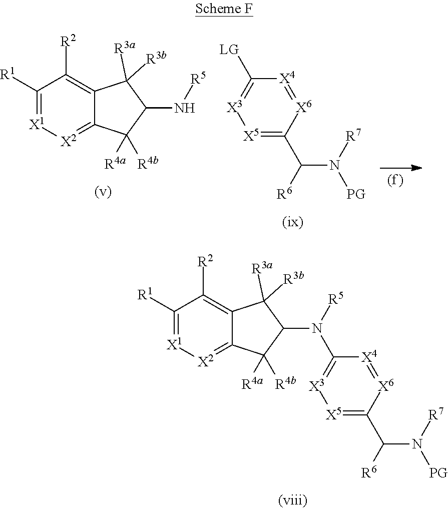

Compounds of Intermediate (viii) can be prepared by the condensation of compounds of Intermediate (v) and Intermediate (ix) in step (f) in the presence of suitable coupling reagent and base (Scheme F). A suitable coupling reagent includes, for example, tBuBrettphosPdG3 in the presence of RuPhos, Pd₂(dba)₃ in the presence of X-Phos, RuPhosPdG3, XPhosPdG2 or BuBrettphosPdG1 in the presence of BrettPhos. A suitable base includes, for example, Cs₂CO₃, K₃CO₃, CsF or sodium t-butoxide. A suitable leaving group (LG) includes, for example, bromide, chloride or triflate.

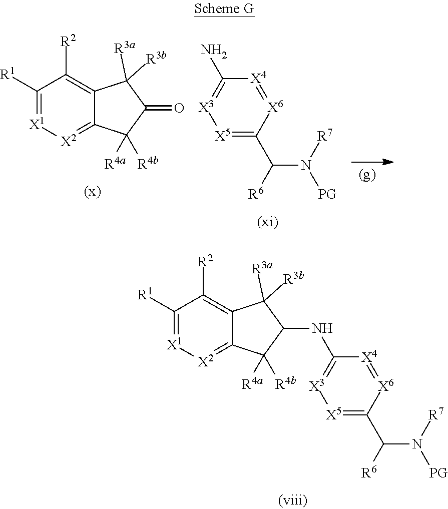

Compounds of Intermediate (viii) may also be prepared by the condensation of compounds of Intermediate (x) and Intermediate (xi) in step (g) with a reductive amination (Scheme G). Suitable conditions include a suitable acid, such as acetic acid, and a suitable reducing reagent including, for example, sodium cyanoborohydride or sodium triacetoxyborohydride.

Compounds of Formula I can also be prepared (Scheme H) by the condensation of compounds of Intermediate (x) and Intermediate (xii) in step (g) using conditions as described in Scheme G. Intermediate (xii) can be prepared by the removal of the benzhydryl of Intermediate (xiii) under suitable conditions (Step h). Suitable conditions include, for example, hydrogenation in the presence of palladium on carbon. Intermediate (xiii) can be prepared from Intermediate (xiv) as outlined above with suitable Intermediate (ii), (iii) or (iv) respectively as described in Scheme D.

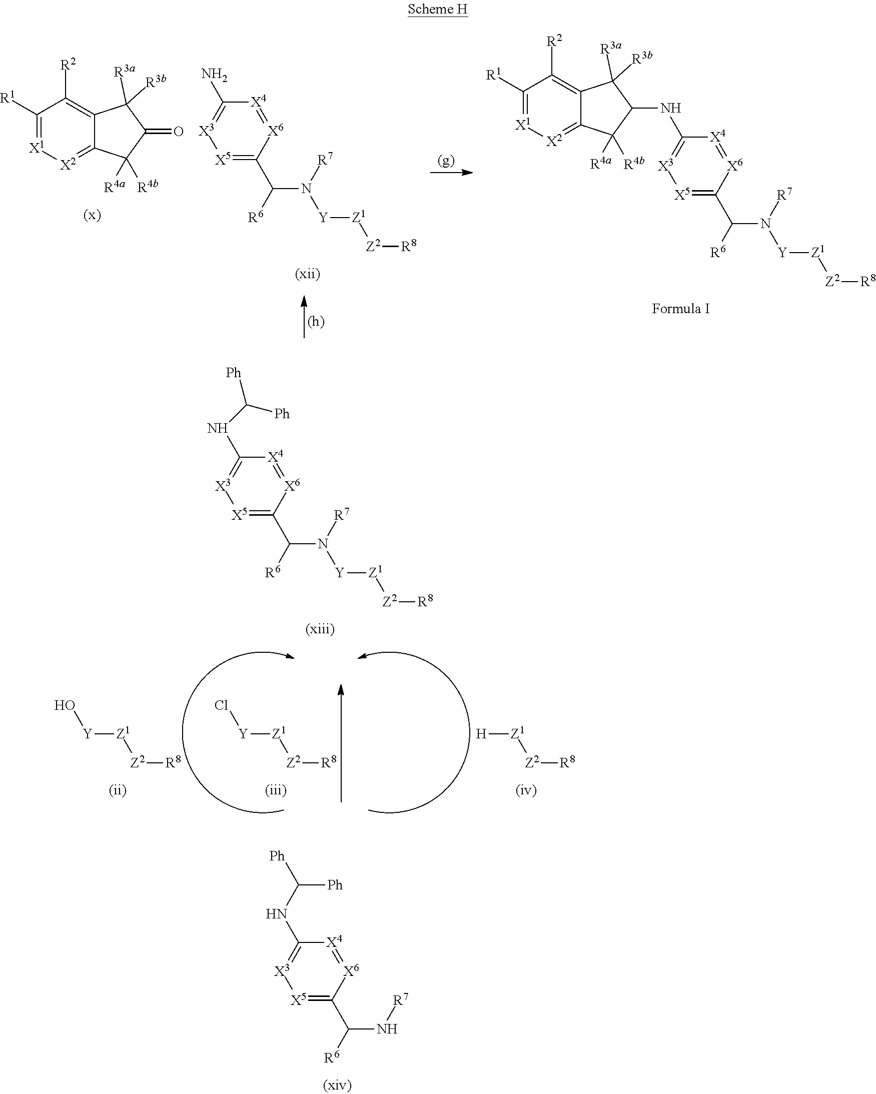

Compounds of Intermediate (xi) can be prepared by the removal of the benzhydryl from Intermediate (xv) in step (k) (Scheme I). Suitable reagents include, for example, formic acid with palladium on carbon or hydroxylamine. Compounds of Intermediate (xv) can be prepared by the condensation of compounds of Intermediate (ix) and benzophenone imine in step (j) in the presence of suitable coupling reagent and base (Scheme I). A suitable coupling reagent includes, for example, tBuBrettphosPdG3 in the presence of RuPhos, Pd₂(dba)₃ in the presence of X-Phos, RuPhosPdG3, XPhosPdG2 or BuBrettphosPdG1 in the presence of BrettPhos. A suitable base includes, for example, Cs₂CO₃, K₂CO₃, CsF or sodium t-butoxide. A suitable leaving group (LG) includes, for example, bromide, chloride or triflate. Compounds of Intermediate (xiv) can be prepared by the concomitant reduction of the imine and removal of the protecting group (PG) of Intermediate (xv) in step (l). A suitable reagent to achieve this transformation includes, for example, lithium aluminium hydride.

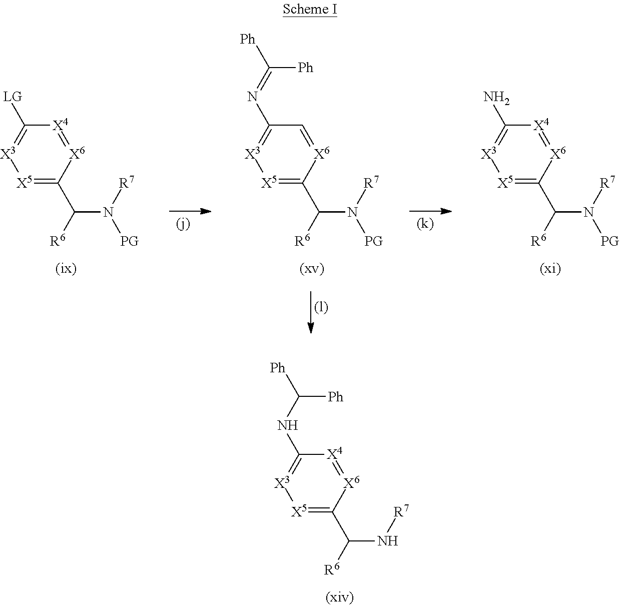

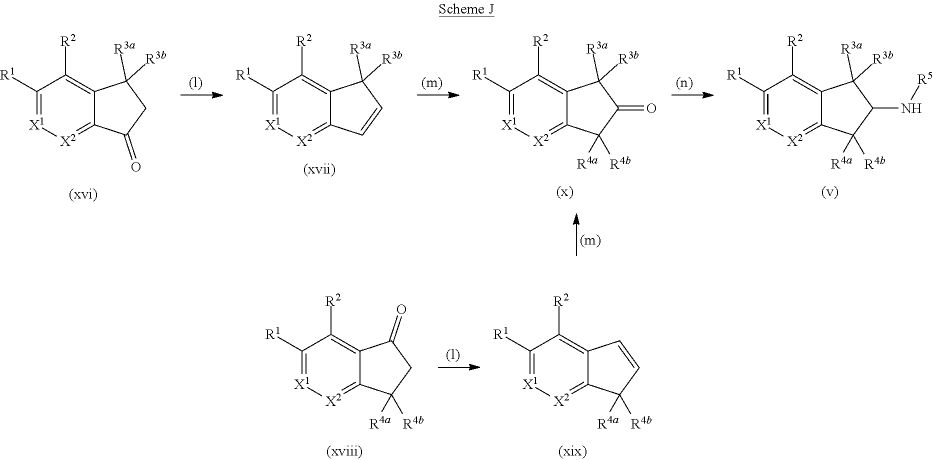

Compounds of Intermediates (v) and (x) can be prepared as outlined in Scheme J. A suitable indan-2-one, as depicted by Intermediate (xvi) or (xviii) can be converted to intermediate (xvii) or (xix) respectively in a two-step process involving reduction of the ketone followed by elimination as in step (l). The reduction of the ketone can be performed, for example, using sodium borohydride or lithium aluminium hydride with the elimination occurring under acidic conditions, for example, treatment with para-toluenesulfonic acid. Intermediate (x) can be prepared by a suitable oxidation procedure (step m) from (xix) or (xvii). An example of such an oxidation procedure would by the two-step process of dihydroxylation followed by acid treatment. A suitable oxidant, for example, would be osmium tetroxide with a suitable acid, for example, being para-toluenesulfonic acid. A further oxidation procedure would be the two-step process of epoxidation followed by acid treatment. A suitable oxidant, for example, would be meta-chloroperoxybenzoic acid with a suitable acid, for example, being silica. The reaction of Intermediate (x) with a suitable amine source and suitable reducing agent under reductive amination conditions (as described in Scheme G) would furnish the required Intermediate (v) (step n). A suitable amine source includes, for example, ammonium acetate or methylamine with a suitable reducing agent, for example, sodium cyanoborohydride or sodium triacetoxyborohydride

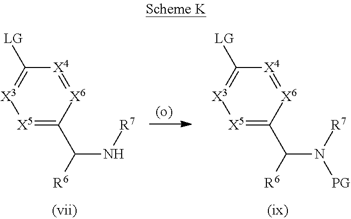

Compounds of Intermediate (ix) can be prepared as outlined in Scheme K. A suitable protecting group including, for example, tert-butoxycarbonyl, pivaloyl or t-butylsulfinyl can be introduced in step (o) with Intermediate (vii) using methods as described in “Protecting groups in Organic Synthesis (3^{rd} Ed), John Wiley & Sons, NY (1999)”, T. Greene & P. Wuts.

Compounds of Intermediate (vii) can be prepared as outlined in Scheme L. Condensation of Intermediate (xx) with t-butylsulfinamide in step (p) affords imine (xxi). Reaction of imine (xxi) with a range of nucleophiles affords Intermediate (xxii) in step q. A suitable nucleophile includes, for example, an organolithium, an organocuprate, a Grignard reagent or, in the case of R⁶ being trifluoromethyl, trifluoromethyltrimethylsilane. The deprotonation of Intermediate (xxii) with a suitable base and reaction with a suitable electrophile will generate Intermediate (xxiii) in step (r). A suitable base includes, for example, sodium hydride, LDA or lithium hexamethyldisilylamide. A suitable electrophile includes, for example, alkyl halide or alkyl triflate. Subsequent removal of the t-butylsulfinyl group using methods as described in “Protecting groups in Organic Synthesis (3^{rd} Ed), John Wiley & Sons, NY (1999)”, T. Greene & P. Wuts affords Intermediate (vii) in step (s).

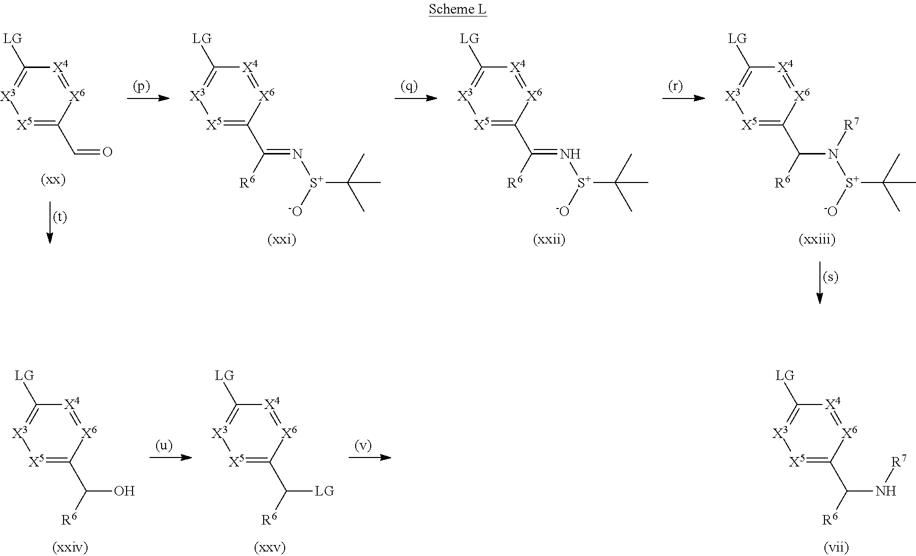

An alternative route to Intermediate (vii) may be achieved as follows (Scheme L). Addition of a suitable nucleophile to Intermediate (xx) affords Intermediate (xxiv) in step (t). A suitable nucleophile includes, for example, an organolithium, an organocuprate, a Grignard reagent, or in the case of R⁶ being trifluoromethyl, trifluoromethyltrimethylsilane. The introduction of a suitable leaving group (LG) to Intermediate (xxiv) in step (u) affords Intermediate (xxv). A suitable leaving group includes, for example, a mesylate, tosylate or triflate. The reaction of the Intermediate (xxv) with a suitable amine nucleophile will afford Intermediate (vii) as in step (v).

### Pharmaceutical Compositions

The compounds of the invention will normally, but not necessarily, be formulated into pharmaceutical compositions prior to administration to a patient. Therefore, according to a further aspect of the invention there is provided a pharmaceutical composition which comprises a compound of the invention as defined hereinbefore, or a pharmaceutically acceptable salt thereof, and one or more pharmaceutically acceptable excipients, diluent or carrier.

The pharmaceutical compositions of the invention may be prepared and packaged in bulk form wherein a safe and effective amount of a compound of the invention can be extracted and then given to the patient such as with powders or syrups. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from 1 mg to 1000 mg.

The compositions of the invention may be in a form suitable for oral use (for example as tablets, capsules, caplets, pills, troches, powders, syrups, elixirs, suspensions, solutions, emulsions, sachets, and cachets), for topical use (for example as creams, ointments, lotions, solutions, pastes, sprays, foams, and gels), for transdermal administration such as via transdermal patches, for administration by inhalation (for example as a dry powders, aerosols, suspensions, and solutions), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must be of sufficiently high purity to render it pharmaceutically-acceptable.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting of the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, humectants, chelating agents, plasticizers, viscosity increasing agents, antioxidants, preservatives, stabilizers, surfactants, and buffering agents. The person skilled in the art will appreciate that certain pharmaceutically-acceptable excipients may serve more than one function and may serve alternative functions depending on how much of the excipient is present in the formulation and what other ingredients are present in the formulation.

Persons skilled in the art possess the knowledge and skill to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general, lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Routes of Administration

The compounds of the invention or pharmaceutical composition comprising the active compound may be administered to a subject by any convenient route of administration, whether systemically/peripherally or topically (i.e. at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

In a preferred embodiment, a compound of the invention as defined herein, or a pharmaceutically acceptable salt thereof, or a pharmaceutical composition as defined herein, is administered orally or via injection, such as conveniently by oral administration.

### Therapeutic Uses and Applications

The compounds of the invention are inhibitors of MALT1. As a consequence, they are potentially useful therapeutic agents for the treatment of diseases or conditions mediated by MALT1.

Thus, in one aspect, the present invention relates to a compound of the invention as defined herein, or a pharmaceutically acceptable salt thereof, or a pharmaceutical composition as defined herein, for use in therapy.

In another aspect, the present invention relates to a compound of the invention as defined herein, or a pharmaceutically acceptable salt thereof, or a pharmaceutical composition as defined herein, for use in the treatment of diseases or disorders mediated by MALT1.

In another aspect, the present invention relates to a method of treating a disease or disorders mediated by MALT1, said method comprising administering to a subject in need of such treatment a therapeutically effective amount of a compound of the invention as defined herein, or a pharmaceutically acceptable salt thereof, or a pharmaceutical composition as defined herein.

Examples of particular diseases or conditions that the compounds of formula (I) and their pharmaceutically acceptable salts may be used to treat include, but are not limited to:
i) lymphomas, leukaemias, carcinomas, and sarcomas; such as non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukaemia (CLL), small lymphocytic lymphoma (SLL), Waldenström macroglobulinemia, lymphoblastic T cell leukaemia, chronic myelogenous leukaemia (CML), hairy-cell leukaemia, acute lymphoblastic T cell leukaemia, plasmacytoma, immunoblastic large cell leukaemia, megakaryoblastic leukaemia, acute megakaryocytic leukaemia, promyelocytic leukaemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, oesophageal cancer, salivary gland cancer, nasopharyngeal cancer, buccal cancer, cancer of the mouth, or GIST (gastrointestinal stromal tumour); and
ii) immunological diseases including autoimmune and inflammatory disorders; such as arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, or polymyositis.

In particular, the compounds of the invention (including pharmaceutically acceptable salts) may be used in the treatment of lymphomas, such as non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, chronic lymphocytic leukaemia (CLL), or small lymphocytic lymphoma (SLL).

In another aspect, the present invention provides a compound, or a pharmaceutically acceptable salt thereof, or a pharmaceutical composition as defined herein, for use in the treatment of non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukaemia (CLL), small lymphocytic lymphoma (SLL), Waldenström macroglobulinemia, lymphoblastic T cell leukaemia, chronic myelogenous leukaemia (CML), hairy-cell leukaemia, acute lymphoblastic T cell leukaemia, plasmacytoma, immunoblastic large cell leukaemia, megakaryoblastic leukaemia, acute megakaryocytic leukaemia, promyelocytic leukaemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, oesophageal cancer, salivary gland cancer, nasopharyngeal cancer, buccal cancer, cancer of the mouth, GIST (gastrointestinal stromal tumour), arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, or polymyositis.

In another aspect, the present invention provides a compound, or a pharmaceutically acceptable salt thereof, or a pharmaceutical composition as defined herein, for use in the treatment of non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, chronic lymphocytic leukaemia (CLL), or small lymphocytic lymphoma (SLL).

In another aspect, the present invention provides a method of treating non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukaemia (CLL), small lymphocytic lymphoma (SLL), Waldenström macroglobulinemia, lymphoblastic T cell leukaemia, chronic myelogenous leukaemia (CML), hairy-cell leukaemia, acute lymphoblastic T cell leukaemia, plasmacytoma, immunoblastic large cell leukaemia, megakaryoblastic leukaemia, acute megakaryocytic leukaemia, promyelocytic leukaemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, oesophageal cancer, salivary gland cancer, nasopharyngeal cancer, buccal cancer, cancer of the mouth, GIST (gastrointestinal stromal tumour), arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, or polymyositis, said method comprising administering to a subject in need of such treatment a therapeutically effective amount of a compound, or a pharmaceutically acceptable salt thereof, or a pharmaceutical composition as defined herein.

In another aspect, the present invention provides a method of treating non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, chronic lymphocytic leukaemia (CLL), or small lymphocytic lymphoma (SLL), said method comprising administering to a subject in need of such treatment a therapeutically effective amount of a compound, or a pharmaceutically acceptable salt thereof, or a pharmaceutical composition as defined herein.

In another aspect, the present invention provides a method of inhibiting MALT1 in vitro, said method comprising administering an effective amount of a compound, or a pharmaceutically acceptable salt thereof.

In another aspect, the present invention provides a method of inhibiting MALT1 in vivo, said method comprising administering an effective amount of a compound, or a pharmaceutically acceptable salt thereof.

In another aspect, the present invention provides a method of inhibiting MALT1 in vitro and/or in vivo, said method comprising contacting a cell with an effective amount of a compound as defined herein, or a pharmaceutically acceptable salt thereof.

### Combination Therapies

The compounds of the invention may be administered alone as a monotherapy or may administered in combination with one or more additional therapeutic agents. The selection of the one or more additional therapeutic agents will of course vary depending on the disease or condition to be treated and its severity.

It is commonplace to use combination therapies to treat certain medical conditions.

According to a particular aspect of the invention there is provided a combination suitable for use in the treatment of a disease or condition in which MALT1 is implicated, comprising a compound of the invention as defined hereinbefore, or a pharmaceutically acceptable salt thereof, and another therapeutic agent.

According to this aspect of the invention there is provided a combination suitable for use in the prevention or treatment of non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukaemia (CLL), small lymphocytic lymphoma (SLL), Waldenström macroglobulinemia, lymphoblastic T cell leukaemia, chronic myelogenous leukaemia (CML), hairy-cell leukaemia, acute lymphoblastic T cell leukaemia, plasmacytoma, immunoblastic large cell leukaemia, megakaryoblastic leukaemia, acute megakaryocytic leukaemia, promyelocytic leukaemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, oesophageal cancer, salivary gland cancer, nasopharyngeal cancer, buccal cancer, cancer of the mouth, GIST (gastrointestinal stromal tumour), arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, or polymyositis, the combination comprising a compound of the invention as defined hereinbefore, or a pharmaceutically acceptable salt thereof, and one or more additional therapeutic agents.

In a further aspect of the invention there is provided a compound of the invention or a pharmaceutically acceptable salt thereof, in combination with one or more additional therapeutic agents.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt thereof in combination with one or more additional therapeutic agents in association with a pharmaceutically acceptable diluent or carrier.

The one or more additional therapeutic agents may comprise a further compound of the present invention. Therefore, in an embodiment, there is provided a pharmaceutical composition which comprises two compounds of the invention, or pharmaceutically acceptable salts thereof, in association with a pharmaceutically acceptable diluent or carrier.

According to a particular aspect of the invention there is provided a combination suitable for use in the prevention or treatment of non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, chronic lymphocytic leukaemia (CLL), or small lymphocytic lymphoma (SLL).

Examples of other therapeutic agents that may be used as part of a combination therapy with a compound of the present invention (e.g. as one of two or more active agents as part of double or triple combinations) include, but are not limited to, the following:
(i) BTK (Bruton's tyrosine kinase) inhibitors such as ibrutinib, acalabrutinib, zanubrutinib, tirabrutinib, orelabrutinib, evobrutinib, fenebrutinib, rilzabrutinib, tolebrutinib, MK1026 (ARQ-531), LOXO-305, elsubrutinib, poseltinib, branebrutinib, spebrutinib, luxeptinib, DTRM-555, JnJ64264681, BGB-3959, AS-1763 and remibrutinib;
(ii) SYK inhibitors such as fostamatinib, entospletinib, HMPL-523, IC-265, SKI-O-703, cerdulatinib, PRT-2761, GSK-264264, SYHX-1901, MK-8457, HM-43239, R-348 and PUR-1800;
(iii) PKC inhibitors such as darovasertib, MS-553, enzastaurin, safingol, ruboxistaurin and AR-13503;
(iv) PI3K pathway inhibitors such as alpelisib, duvelisib, copanlisib, idelalisib, umbralisib, serabelisib, CHF-6523, BDP-681, zandelisib, ART-001, buparlisib, OP-11, HMPL-689, dezapelisib, seletalisib, epivotide, IOA-244, SHC-014748M, LX-086, inavolisib, MEN-1611, eganelisib, leniolisib, ACP-319, BGB-10188, CYH-33, HS-10352, CMX-2043, ZX-101A, KA-2237, VS-5584, ASN-003, TQ-B325, AL-58805, gedatolisib, HEC-68498, CLL-442, tenalisib, dactolisib, GSK-2126458 and bimiralisib;
(v) Bcl family inhibitors such as ABT-737, HA14-1, BH3I-1, A-1155463, A-1331852, A-1210477, BDA-366, TW-37, S44563, S64315 (MIK665), S63845, BCL-201, AMG176, AZD-0466, AZD5991, UMI-77, navitoclax, pelcitoclax, obatoclax, sabutoclax, apogossypol, gossypol, antimycin A, Gambogic acid, LP-118, FCN-338, BGB-11417, UBX-1325, LP-108, VOB-560, lisaftoclax, murizatoclax, venetoclax, ZN-d5 and ABBV-167;
(vi) JAK inhibitors such as gusacitinib, delgocitinib, tofacitinib, abrocitinib, ruxolitinib, baricitinib, fedratinib, upadacitinib, filgotinib, peficitinib, TD-8236, TD-0903, CEE-321, lorpucitinib, WXSH-0150, SYHX-1901, cerdulatinib, izencitinib, KL-130008, WP-1066, gusacitinib, INCB-52793, AC-1101, ATI-1777, SHR-0302, CPL-409116, momelotinib, brepocitinib, TTL-018, TD-5202, LP-0184, INCB-054707, jaktinib, TQ-05105, itacitinib, AZD-0449, GLPG-0555, LW-104, ARQ-252, WXFL-10203614, golidocitinib, deuruxolitinib, CJ-15314, CS-12192, ritlecitinib, R-348, ATI-2138 and ilginatinib;
(vii) PIM kinase inhibitors such as uzansertib, TP-3654, MEN-1703, ETH-155008, abemaciclib and SF-1126;
(viii) mTORC inhibitors such as rapamycin, sirolimus, novolimus, umirolimus zotarolimus, temsirolimus, everolimus, merilimus, eRapa, ridaforolimus;
(ix) Rituximab or other B cell antigen-binding antibodies as well as immune cell redirection agents (e.g. blinatumomab or CAR-T cells);
(x) Anti-PD1 antibodies such as nivolumab, pembrolizumab, lambrolizumab, pidilizumab, BGB-A317;
(xi) Anti-PD-L1 antibodies such as atezolizumab, avelumab, durvalumab, MEDI-4736 and MPDL3280A;
(xii) Antibodies that inhibit the 4-1BB-ligand interaction such as utomilumab; and
(xiii) Antibodies that inhibit the interaction of CTLA-4 and its ligands such as ipilumumab, tremelimumab, or those disclosed in WO2014/207063.

The combinations referred to above may conveniently be presented for use in the form of a pharmaceutical formulation and thus pharmaceutical formulations comprising a combination as defined above together with a pharmaceutically acceptable diluent or carrier represent a further aspect of the invention.

Such conjoint/combination treatment may be achieved by way of the simultaneous, sequential or separate dosing of the individual components of the treatment. In one embodiment, the individual compounds will be administered simultaneously in a combined pharmaceutical formulation.

Such combination therapies employ the compounds of this invention within the dosage range described herein and the other pharmaceutically active agent within approved dosage ranges and/or the dosage such as described in the relevant publication reference.

### EXAMPLES

### General Procedures:

Methods for preparing the compounds of this invention are illustrated in the following Examples. Starting materials are made according to procedures known in the art or as illustrated herein or are available commercially. Commercial reagents were used without further purification. Where no reaction temperature is included, the reaction was performed at ambient temperature which is typically 17-27° C.

A person skilled in the art will appreciate that reaction temperatures, reaction times & reagent quantities may be varied from those stated herein.

Where compounds described in the invention are characterized by ¹H NMR spectroscopy, spectra were recorded on 400 MHz Bruker Avance III HD instrument and 300 MHz Bruker Fourier HD instrument. Where no temperature is included, the spectra were recorded at ambient temperature. Chemical shift values are expressed in parts per million (ppm). The following abbreviations are used for the multiplicity of the NMR signals: s=singlet, br=broad, t=triplet, q=quartet, m=multiplet, d=doublet.

Where compounds described in the invention are characterized by LCMS data, molecular weight is determined using the conditions listed below.
Method A: Agilent 1260 (Waters Acquity PDA 210-400 nm and Waters Acquity SQ detector). Column: Waters Acquity BEH C18, 130 Å, 1.7 μm, 50×2.1 mm. Conditions: 0.1% formic acid in water [eluent A], 0.1% formic acid in MeCN [eluent B].
Method B: Agilent 1260 (Waters Acquity PDA 210-400 nm and Waters Acquity SQ detector). Column: XBridge C18, 130 Å, 2.5 μm, 50×2.1 mm. Conditions: 10 mM ammonium acetate in water [eluent A], MeCN [eluent B].
Method C: Agilent 1260 (Waters Acquity PDA 210-400 nm and Waters Acquity SQ detector). Column: Waters Acquity BEH C18, 130 Å, 1.7 μm, 50×2.1 mm. Conditions: 0.05% formic acid in water [eluent A], 0.05% formic acid in MeCN [eluent B].
Method D: Agilent 1260 (Waters Acquity PDA 210-400 nm and Waters Acquity SQ detector). Column: Ascentis Express C18, 2.7 μm, 50×2.1 mm. Conditions: 0.1% formic acid in water [eluent A], 0.1% formic acid in MeCN [eluent B].
Method E: Agilent 1260 (Waters Acquity PDA 210-400 nm and Waters Acquity SQ detector). Column: Waters Acquity BEH C18, 130 Å, 1.7 μm, 50×2.1 mm. Conditions: 10 mM ammonium acetate in water [eluent A], MeCN [eluent B].
Method F: Agilent 1260 (Waters Acquity PDA 210-400 nm and Waters Acquity SQ detector). Column: Waters Acquity BEH C18, 130 Å, 3.5 μm, 50×4.6 mm. Conditions: 5 mM NH₄HCO₃ in water [eluent A], MeCN [eluent B].
Method G: Agilent 1260 (Waters Acquity PDA 210-400 nm and Waters Acquity SQ detector). Column: Waters Acquity BEH C18, 130 Å, 1.7 μm, 50×2.1 mm. Conditions: 5 mM ammonium acetate in water [eluent A], MeCN [eluent B].
Method H: Agilent 1260 (Waters Acquity PDA 210-400 nm and Waters Acquity SQ detector). Column: Waters Acquity BEH C18, 130 Å, 1.7 μm, 50×2.1 mm. Conditions: 0.1% TFA in water [eluent A], 0.1% TFA in MeCN [eluent B].
Method I: Agilent 1260 (Waters Acquity PDA 210-400 nm and Waters Acquity SQ detector). Column: Waters Acquity BEH C18, 130 Å, 1.7 μm, 100×2.1 mm. Conditions: 0.1% TFA in water [eluent A], 0.1% TFA in MeCN [eluent B].
Method J: Shimadzu 2020 (SPD-M40 PDA 220/254 nm and MS detector). Column: Waters Acquity BEH C18, 130 Å, 1.7 μm, 100×2.1 mm. Conditions: 0.1% formic acid in water [eluent A], 0.1% formic acid in MeCN [eluent B].
Method K: Shimadzu 2020 (SPD-M40 PDA 254/280 nm and MS detector). Column: Waters Acquity BEH C18, 130 Å, 1.7 μm, 100×2.1 mm. Conditions: 0.1% formic acid in water [eluent A], 0.1% formic acid in MeCN [eluent B].
Method L: Waters Acquity I-Class Plus (Waters Acquity PDA 210-400 nm and Waters Acquity SQ detector). Column: Waters Acquity BEH C18, 130 Å, 1.7 μm, 100×2.1 mm. Conditions: 0.1% formic acid in water [eluent A], 0.1% formic acid in MeCN [eluent B].
Method M: Dionex UHPLC Ultimate 3000 (DAD 190-340 nm and Thermo Scientific ISQ EC detector). Column: Kinetex XB-C18, 110 Å, 2.6 μm, 50×4.6 mm. Conditions: 0.1% formic acid in water [eluent A], 0.1% formic acid in MeCN [eluent B].
Method N: Dionex UHPLC Ultimate 3000 (DAD 190-340 nm and Thermo Scientific ISQ EC detector). Column: Kinetex XB-C18, 110 Å, 2.6 μm, 50×4.6 mm. Conditions: 0.1% ammonia in water [eluent A], MeCN [eluent B].
Method O: Agilent 1260 (Waters Acquity PDA 210-400 nm and Waters Acquity SQ detector). Column: XBridge C18, 130 Å, 3.5 μm, 100×4.6 mm. Conditions: 10 mM NH₄HCO₃ in water [eluent A], MeCN [eluent B].
Method P: Agilent 1260 (Waters Acquity PDA 210-400 nm and Waters Acquity SQ detector). Column: XBridge BEH C18, 130 Å, 2.5 μm, 50×2.1 mm. Conditions: 5 mM NH₄HCO₃ in water [eluent A], MeCN [eluent B].
Method Q: Agilent 1260 (Waters Acquity PDA 210-400 nm and Waters Acquity SQ detector). Column: YMC-Triart C18, 120 Å, 1.9 μm, 50×2.1 mm. Conditions: 10 mM ammonium acetate in water [eluent A], MeCN [eluent B].
Method R: Agilent 1260 (Waters Acquity PDA 210-400 nm and Waters Acquity SQ detector). Column: YMC-Triart C18, 120 Å, 1.9 μm, 50×2.1 mm. Conditions: 0.1% formic acid in water [eluent A], MeCN [eluent B].
Method S: Agilent 1260 (Waters Acquity PDA 210-400 nm and Waters Acquity SQ detector). Column: X-Select CSH C18, 5 μm, 100×4.6 mm. Conditions: 10 mM ammonium acetate in water [eluent A], MeCN [eluent B].
Method T: Dionex UHPLC Ultimate 3000 (DAD 190-340 nm and Thermo Scientific ISQ EC detector). Column: Kinetex XB-C18, 110 Å, 2.6 μm, 50×4.6 mm. Conditions: water [eluent A], MeCN [eluent B].

Preparative HPLC was performed using Shimadzu CBM-20A/SIL-10AP; Shimadzu Nexera; Waters Delta; Gilson GX-271; Agilent 1260 Infinity II.

Preparative SFC was performed using Waters SFC-150-I; Water SFC-150-II; Waters SFC-200; Sepiatec-200.

Automated purification was carried out using Interchim Puriflash XS 520: Normal-phase PF-50SIHP-JP; Reverse-phase PF-15C18HP.

### Abbreviations

| | |
|---|---|
| AcOH | Acetic acid |
| Boc | tert-Butyloxycarbonyl |
| BrettPhos | 2-(Dicyclohexylphosphino)3,6-dimethoxy-2′,4′,6′-triisopropyl-1,1′- |
| | biphenyl |
| BrettPhosPdG1 | Chloro[2-(dicyclohexylphosphino)-3,6-dimethoxy-2′,4′,6′-triisopropyl- |
| | 1,1′-biphenyl][2-(2-aminoethyl)phenyl]palladium(II) |
| Cs₂CO₃ | Cesium carbonate |
| DBU | 1,8-Diazabicyclo[5.4.0]undec-7-ene |
| DCE | 1,2-Dichloroethane |
| DCM | Dichloromethane |
| DIPEA | N,N-Diisopropylethylamine |
| DMAP | 4-Dimethylaminopyridine |
| DMF | N,N-Dimethylformamide |
| DMSO | Dimethylsulfoxide |
| EDCI | 1-Ethyl-3-(3-dimethylaminopropyl)carbodiimide |
| EtOAc | EtOAc |
| h | Hour (s) |
| HATU | 1-[Bis(dimethylamino)methylene]-1H-1,2,3-triazolo[4,5-b]pyridinium 3- |
| | oxid hexafluorophosphate |
| HOBt | Hydroxybenzotriazole |
| HCl | Hydrogen chloride |
| HPLC | High Performance Liquid Chromatography |
| IPA | Isopropyl alcohol |
| K₂CO₃ | Potassium carbonate |
| K₃PO₄ | Potassium phosphate tribasic |
| LCMS | Liquid Chromatography Mass Spectrometry |
| LDA | Lithium diisopropylethylamide |
| LiAlH₄ | Lithium aluminium hydride |
| MCPBA | Meta-Chloroperoxybenzoic acid |
| MeCN | Acetonitrile |
| MeOH | Methanol |
| MeTHF | 2-Methyltetrahydrofuran |
| MgSO₄ | Magnesium sulfate |
| min | minute(s) |
| NaHCO₃ | Sodium bicarbonate |
| NaOH | Sodium hydroxide |
| Na₂SO₄ | Sodium sulfate |
| NH₄Cl | Ammonium chloride |
| NH₄HCO₃ | Ammonium bicarbonate |
| NH₄OH | Ammonium hydroxide |
| NMMO | N-Methylmorpholine-N-oxide |
| NMR | Nuclear Magnetic Resonance |
| Pd₂(dba)₃ | Tris(dibenzylideneacetone)dipalladium(0) |
| Pd(dppf)Cl₂ | [1,1′-Bis(diphenylphosphino)ferrocene]palladium(II)dichloride |
| Pd(dppf)Cl₂•DCM | [1,1′-Bis(diphenylphosphino)ferrocene]palladium(II)dichloride |
| | dichloromethane adduct |
| Pd-PEPPSI-iHeptCl | Dichloro[1,3-bis(2,6-di-4-heptylphenyl)imidazol-2-ylidene](3- |
| | chloropyridyl)palladium(II). |
| POCl₃ | Phosphorous oxychloride |
| Prep-TLC | Preparative thin layer chromatography |
| pTSA | p-Toluene sulfonic acid monohydrate |
| RT | Room temperature |
| RuPhos | 2-Dicyclohexylphosphino-2′,6′-diisopropoxybiphenyl |
| RuPhosPdG3 | (2-Dicyclohexylphosphino-2′,6′-diisopropoxy-1,1′-biphenyl)[2-(2′-amino- |
| | 1,1′-biphenyl)]palladium(II) methanesulfonate |
| s | Second (s) |
| SFC | Supercritical Fluid Chromatography |
| SPE | Solid phase extraction |
| tBuBrettphosPdG3 | [(2-Di-tert-butylphosphino-3,6-dimethoxy-2′,4′,6′-triisopropyl-1,1′- |
| | biphenyl)-2-(2′-amino-1,1′-biphenyl)]palladium(II) methanesulfonate |
| tBuOH | Tert-Butanol |
| T3P ® | Propylphosphonic anhydride |
| TBAF | Tetra-N-butylammonium fluoride |
| TBDME | Tert-Butyl dimethyl ether |
| TBDMS-Cl | Tert-Butyldimethylsilyl chloride |
| TFA | Trifluoroacetic acid |
| TFMTMS | Trimethyl(trifluoromethyl)silane |
| THF | Tetrahydrofuran |
| Xantphos | 4,5-Bis(diphenylphosphino)-9,9-dimethylxanthene |
| X-Phos | 2-Dicyclohexylphosphino-2′,4′,6′-triisopropylbiphenyl |
| XPhosPdG2 | Chloro(2-dicyclohexylphosphino-2′,4′,6′-triisopropyl-1,1′-biphenyl)[2-(2′- |
| | amino-1,1′-biphenyl)]palladium(II) |
| XPhosPdG3 | (2-Dicyclohexylphosphino-2′,4′,6′-triisopropyl-1,1′-biphenyl)[2-(2′-amino- |
| | 1,1′-biphenyl)]palladium(II) methanesulfonate |

### INTERMEDIATE SYNTHESIS

### Intermediate 1: (R)—N—((S)-1-(4-Bromophenyl)-2,2,2-trifluoroethyl)-N,2-dimethylpropane-2-sulfinamide

To a stirred solution of (R)—N—((S)-1-(4-bromophenyl)-2,2,2-trifluoroethyl)-2-methylpropane-2-sulfinamide (5.0 g, 14.0 mmol, CAS 336105-31-8) in 2-methyltetrahydrofuran (50 mL) at −78° C. was added LDA (2M in THF, 21 mL, 42.0 mmol) and the mixture was stirred for 1 h. Iodomethane (11.9 g, 83.7 mmol) was added then the mixture was allowed to reach RT slowly and then stirred for 12 h. The mixture was cooled to 0° C. then quenched with saturated aqueous NH₄Cl, diluted with water, then extracted with EtOAc. The organic layer was dried over Na₂SO₄, filtered and concentrated under reduced pressure. The crude product was purified by flash chromatography (silica gel, eluting 10-15% EtOAc in petroleum ether) to provide the title compound (2.5 g). LCMS (Method A): 372.2 [M+H]⁺.

### Intermediate 2: (S)-1-(4-Bromophenyl)-2,2,2-trifluoro-N-methylethan-1-amine Hydrochloride

To a stirred solution of Intermediate 1 (3.3 g, 8.87 mmol) in EtOAc (5 mL), was added 4M HCl in 1,4-dioxane (10 mL) at RT and the mixture was stirred for 16 h. The mixture was concentrated under reduced pressure. The crude product was triturated in n-pentane and filtered to provide the title compound (2.7 g). LCMS (Method A): 268.0 [M+H]⁺.

### Intermediate 3: (S)—N-(1-(4-Bromophenyl)-2,2,2-trifluoroethyl)-N-methyltetrahydro-2H-thiopyran-4-carboxamide 1,1-dioxide

To a stirred solution of Intermediate 2 (3.3 g, 8.87 mmol) in pyridine (2.0 mL, 24.6 mmol) was added T3P® (50% EtOAc, 26.1 g, 82.1 mmol) and tetrahydro-2H-thiopyran-4-carboxylic acid 1,1-dioxide (1.46 g, 8.21 mmol, CAS 64096-87-3) and the mixture was stirred at RT for 16 h. The mixture was diluted with water, then extracted with EtOAc. The organic layer was dried over Na₂SO₄, filtered and concentrated under reduced pressure. The crude product was purified by flash chromatography (silica gel, eluting 50-55% EtOAc in petroleum ether) to provide the title compound (2.87 g). LCMS (Method A): 428.2 [M+H]⁺.

### Intermediate 4: (S)—N-(1-(4-Bromophenyl)-2,2,2-trifluoroethyl)-N-methyloxazole-5-carboxamide

To a stirred solution of Intermediate 2 (3.3 g, 8.87 mmol) in pyridine (5.0 mL, 24.6 mmol) was added oxazole-5-carboxylic acid (0.17 g, 1.48 mmol, CAS 118994-90-4) and DMAP (12 mg, 0.01 mmol) and stirred at RT for 10 min. The mixture was cooled at 0° C. then POCl₃ (0.15 g, 0.99 mmol) was added and the mixture was stirred at RT for 1 h. The mixture was diluted with water, then extracted with EtOAc. The organic layer was dried over Na₂SO₄, filtered and concentrated under reduced pressure. The crude product was purified by flash chromatography (neutral alumina, eluting 40% EtOAc in petroleum ether) to provide the title compound (0.38 g). LCMS (Method A): 363.0 [M+H]⁺.

### Intermediate 5: (S)-1-Acetyl-N-(1-(4-bromophenyl)-2,2,2-trifluoroethyl)-N-methylazetidine-3-carboxamide

The title compound (0.45 g) was prepared in an analogous manner to Intermediate 4 from Intermediate 2 (0.4 g, 1.30 mmol), 1-acetylazetidine-3-carboxylic acid (0.28 g, 0.15 mmol, CAS 97628-91-6), DMAP (10 mg, 0.01 mmol), POCl₃ (0.2 g, 0.13 mmol) in pyridine (10 mL) at RT for 2 h. Purified by flash chromatography (silica gel, eluting 70-100% EtOAc in petroleum ether). LCMS (Method A): 392.9 [M+H]⁺.

### Intermediate 6: 1-Methyl-2,3-dihydro-1H-inden-2-amine Hydrochloride

To a stirred solution of 1-methyl-1,3-dihydro-2H-inden-2-one (0.45 g, 3.08 mmol, CAS 35587-60-1) in ethanol (5.0 mL) was added ammonium acetate (3.56 g, 46.2 mmol) and sodium cyanoborohydride (0.23 g, 3.69 mmol) and stirred at 80° C. for 4 h. The mixture was cooled to 0° C. then 1M aqueous HCl was added to adjust pH to 3 then concentrated under reduced pressure. The crude product was purified by preparative HPLC (ATLANTIS T319, 19×250 mm×5 μm, flow rate: 18 mL/min, 0.05% HCl in water with MeCN 10% to 35% over 7 min, held at 35% for 1 min then ramped to 98% over 0.1 min and held for 6.9 min) to provide the title compound (0.32 g) LCMS (Method A): 148.2 [M+H]⁺.

### Intermediate 7: (S)—N-(1-(4-Bromophenyl)-2,2,2-trifluoroethyl)-N-methylmorpholine-4-carboxamide

To a stirred solution of triphosgene (0.39 g, 1.32 mmol) in DCM (5 mL) at 0° C. was added Intermediate 2 (0.4 g, 1.30 mmol) and stirred for 5 min. Triethylamine (0.6 mL, 3.93 mmol) was added followed by morpholine (0.12 mL, 1.32 mmol) and stirred at 0° C. for 1 h. The mixture was quenched with ice cold water, then extracted with DCM. The organic layer was dried over Na₂SO₄, filtered and concentrated under reduced pressure. The crude product was purified by flash chromatography (silica gel, eluting 30-70% EtOAc in petroleum ether) to provide the title compound (0.45 g). LCMS (Method A): 381.0 [M+H]⁺.

### Intermediate 8: (S)—N-(1-(4-Bromophenyl)-2,2,2-trifluoroethyl)-N-methylthiomorpholine-4-carboxamide 1,1-dioxide

The title compound (0.35 g) was prepared in an analogous manner to Intermediate 7 from Intermediate 2 (0.3 g, 0.99 mmol), thiomorpholine-1,1-dioxide (0.16 g, 1.19 mmol), triethylamine (0.43 mL, 2.97 mmol), triphosgene (0.32 g, 1.09 mmol) in DCM (5 mL) at 0° C. for 1 h. Purified by flash chromatography (silica gel, eluting 30-70% EtOAc in petroleum ether). LCMS (Method A): 429.0 [M+H]⁺.

### Intermediate 9: (S)—N-(1-(4-Bromophenyl)-2,2,2-trifluoroethyl)-4-hydroxy-N,4-dimethylpiperidine-1-carboxamide

The title compound (0.35 g) was prepared in an analogous manner to Intermediate 7 from Intermediate 2 (0.35 g, 1.15 mmol), 4-methylpiperidin-4-ol (0.14 g, 1.21 mmol), triethylamine (0.50 mL, 3.45 mmol), triphosgene (0.37 g, 1.21 mmol) in DCM (5 mL) at 0° C. for 1 h. Purified by flash chromatography (silica gel, eluting 30-70% EtOAc in petroleum ether). LCMS (Method A): 411.0 [M+H]⁺.

### Intermediate 10: (S)—N-(1-(4-Bromophenyl)-2,2,2-trifluoroethyl)-N,4-dimethyl-4-((trimethylsilyl)oxy)piperidine-1-carboxamide

To a stirred solution of Intermediate 9 (0.35 g, 1.32 mmol) in DCM (5 mL) at 0° C. was added TFMTMS (0.19 g, 1.71 mmol), triethylamine (0.37 mL, 2.58 mmol) and DMAP (5 mg, 0.04 mmol). The mixture was stirred at RT for 3 h then quenched with ice cold water and extracted with DCM. The organic layer was dried over Na₂SO₄, filtered and concentrated under reduced pressure. The crude product was purified by flash chromatography (silica gel, eluting 10-50% EtOAc in petroleum ether) to provide the title compound (0.30 g). LCMS (Method A): 483.1 [M+H]⁺.

### Intermediate 11: N-((1S)-1-(4-((1,1-dimethyl-2,3-dihydro-1H-inden-2-yl)amino)phenyl)-2,2,2-trifluoroethyl)-N,4-dimethyl-4-((trimethylsilyl)oxy)piperidine-1-carboxamide

To a stirred solution of Intermediate 10 (0.35 g, 1.32 mmol) and 1,1-dimethyl-2,3-dihydro-1H-inden-2-amine hydrochloride (0.12 g, 0.62 mmol, CAS 74413-86-8) in 1,4-dioxane (3 mL) was added Cs₂CO₃ (0.50 g, 1.55 mmol) and the mixture was degassed with nitrogen for 5 min. tBuBrettphosPdG3 (0.04 g, 0.04 mmol) and RuPhos (0.04 g, 0.09 mmol) were added and the mixture was heated at 120° C. under microwave irradiation for 2 h. The mixture was allowed to cool then diluted with water and extracted with EtOAc. The organic layer was dried over Na₂SO₄, filtered and concentrated under reduced pressure. The crude product was purified by flash chromatography (silica gel, eluting 10-50% EtOAc in petroleum ether) to provide the title compound (0.15 g). LCMS (Method A): 562.4 [M+H]⁺.

### Intermediate 12: (S)—N-(1-(4-Bromophenyl)-2,2,2-trifluoroethyl)-N-methyltetrahydro-2H-pyran-4-carboxamide

The title compound (0.18 g) was prepared in an analogous manner to Intermediate 4 from Intermediate 2 (0.7 g, 2.29 mmol), tetrahydro-2H-pyran-4-carboxylic acid (0.45 g, 3.45 mmol, CAS 5337-03-1), DMAP (5 mg, 0.04 mmol), POCl₃ (0.32 mL, 3.43 mmol) in pyridine (10 mL) at RT for 2 h. Purified by flash chromatography (silica gel, eluting 70-100% EtOAc in petroleum ether). LCMS (Method A): 382.0 [M+H]⁺.

### Intermediate 13: (S)—N-((1S)-1-(4-((1,1-Dimethyl-2,3-dihydro-1H-inden-2-yl)amino)phenyl)-2,2,2-trifluoroethyl)-N,2-dimethylpropane-2-sulfinamide

The title compound (0.17 g) was prepared in an analogous manner to Intermediate 11 from Intermediate 1 (0.4 g, 1.07 mmol), 1,1-dimethyl-2,3-dihydro-1H-inden-2-amine hydrochloride (0.21 g, 1.08 mmol, CAS 74413-86-8), Cs₂CO₃ (1.40 g, 4.30 mmol), tBuBrettphosPdG3 (0.18 g, 0.22 mmol) and RuPhos (0.2 g, 0.43 mmol) in 1,4-dioxane (10 mL) at 120° C. for 2 h. Product used crude. LCMS (Method A): 453.2 [M+H]⁺.

### Intermediate 14: 1,1-Dimethyl-N-(4-((S)-2,2,2-trifluoro-1-(methylamino)ethyl)phenyl)-2,3-dihydro-1H-inden-2-amine Hydrochloride

To a stirred solution of Intermediate 13 (0.17 g, 0.37 mmol) in EtOAc (5 mL), was added 4M HCl in 1,4-dioxane (5 mL) at 0° C. and the mixture was stirred at RT for 3 h. The mixture was concentrated under reduced pressure. The crude product was triturated in diethyl ether and filtered to provide the title compound (0.12 g). LCMS (Method A): 349.6 [M+H]⁺.

### Intermediate 15: (S)—N-(1-(4-Bromophenyl)-2,2,2-trifluoroethyl)-4-hydroxy-N-methylpiperidine-1-carboxamide

The title compound (0.3 g) was prepared in an analogous manner to Intermediate 7 from Intermediate 2 (0.13 g, 0.30 mmol), piperidin-4-ol (0.03 g, 0.30 mmol), triethylamine (0.05 g, 0.30 mmol), triphosgene (0.06 g, 0.21 mmol) in DCM (8 mL) at RT for 2 h. Purified by flash chromatography (silica gel, eluting 40% EtOAc in petroleum ether). LCMS (Method A): 395.1 [M+H]⁺.

### Intermediate 16: (S)—N-(1-(4-Bromophenyl)-2,2,2-trifluoroethyl)-N-methyl-4-((trimethylsilyl)oxy)piperidine-1-carboxamide

To a stirred solution of Intermediate 15 (0.30 g, 0.76 mmol) in DCM (10 mL) at 0° C. was added DIPEA (0.37 mL, 2.58 mmol), then after 10 min, trimethylsilyl chloride (0.16 g, 1.52 mmol). The mixture was stirred at RT for 2 h then quenched with water and extracted with DCM. The organic layer was dried over Na₂SO₄, filtered and concentrated under reduced pressure. The crude product was purified by flash chromatography (silica gel, eluting 5% EtOAc in petroleum ether) to provide the title compound (0.30 g). LCMS (Method B): 467.4 [M+H]⁺.

### Intermediate 17: (S)-3-Acetamido-N-(1-(4-bromophenyl)-2,2,2-trifluoroethyl)-N-methylpropanamide

The title compound (0.35 g) was prepared in an analogous manner to Intermediate 3 from Intermediate 2 (0.3 g, 0.99 mmol), pyridine (0.24 mL, 2.96 mmol), T3P® (50% EtOAc, 4.38 mL, 6.88 mmol) and 3-acetamidopropanoic acid (0.19 g, 1.48 mmol, CAS 3025-95-4) at RT for 12 h. The crude product was purified by flash chromatography (silica gel, eluting 40% EtOAc in petroleum ether). LCMS (Method A): 381.0 [M+H]⁺.

### Intermediate 18: (S)—N-(1-(4-Bromophenyl)-2,2,2-trifluoroethyl)-N-methyloxazole-4-carboxamide

The title compound (0.35 g) was prepared in an analogous manner to Intermediate 4 from Intermediate 2 (0.35 g, 1.15 mmol), oxazole-4-carboxylic acid (0.16 g, 1.38 mmol, CAS 23012-13-7), POCl₃ (0.17 mL, 0.172 mmol) in pyridine (3 mL) at RT for 3 h. Purified by flash chromatography (silica gel, eluting 50% EtOAc in petroleum ether). LCMS (Method A): 363.1 [M+H]⁺.

### Intermediate 19: (S)—N-(1-(4-Bromophenyl)-2,2,2-trifluoroethyl)-2-methoxy-N-methylacetamide

The title compound (0.25 g) was prepared in an analogous manner to Intermediate 4 from Intermediate 2 (0.5 g, 1.64 mmol), 2-methoxyacetic acid (0.18 g, 1.97 mmol, CAS 625-45-6), DMAP (20 mg, 0.23 mmol), POCl₃ (0.37 g, 2.46 mmol) in pyridine (2.5 mL) at RT for 1 h. Purified by flash chromatography (silica gel, eluting 20-25% EtOAc in petroleum ether). LCMS (Method A): 340.0 [M+H]⁺.

### Intermediate 20: N-((1S)-1-(4-((1,1-Dimethyl-2,3-dihydro-1H-inden-2-yl)amino)phenyl)-2,2,2-trifluoroethyl)-2-methoxy-N-methylacetamide

The title compound (0.10 g) was prepared in an analogous manner to Intermediate 11 from Intermediate 19 (0.3 g, 0.89 mmol), 1,1-dimethyl-2,3-dihydro-1H-inden-2-amine hydrochloride (0.17 g, 0.89 mmol, CAS 74413-86-8), Cs₂CO₃ (1.15 g, 3.54 mmol), tBuBrettphosPdG3 (75 mg, 0.09 mmol) and RuPhos (82 mg, 0.18 mmol) in 1,4-dioxane (3 mL) at 120° C. for 2 h. Purified by flash chromatography (silica gel, eluting 20% EtOAc in petroleum ether). LCMS (Method A): 421.2 [M+H]⁺.

### Intermediate 21: (S)—N-(1-(4-Bromophenyl)-2,2,2-trifluoroethyl)-3-methoxy-N-methylpropanamide

The title compound (0.30 g) was prepared in an analogous manner to Intermediate 4 from Intermediate 2 (0.40 g, 1.30 mmol), 3-methoxypropanoic acid (0.27 g, 2.65 mmol, CAS 2544-06-1), DMAP (20 mg, 0.23 mmol), POCl₃ (0.20 g, 1.30 mmol) in pyridine (10 mL) at RT for 2 h. Purified by flash chromatography (silica gel, eluting 50-100% EtOAc in petroleum ether). LCMS (Method A): 354.0 [M+H]⁺.

### Intermediate 22: N-((1S)-1-(4-((1,1-Dimethyl-2,3-dihydro-1H-inden-2-yl)amino)phenyl)-2,2,2-trifluoroethyl)-3-methoxy-N-methylpropanamide

The title compound (0.20 g) was prepared in an analogous manner to Intermediate 11 from Intermediate 21 (0.3 g, 0.85 mmol), 1,1-dimethyl-2,3-dihydro-1H-inden-2-amine hydrochloride (0.17 g, 0.89 mmol, CAS 74413-86-8), Cs₂CO₃ (0.83 g, 2.55 mmol), tBuBrettphosPdG3 (34 mg, 0.04 mmol) and RuPhos (40 mg, 0.09 mmol) in 1,4-dioxane (5 mL) at 120° C. for 2 h. Purified by flash chromatography (silica gel, eluting 70-100% EtOAc in petroleum ether). LCMS (Method A): 435.3 [M+H]⁺.

### Intermediate 23: 1-(6-Bromopyridin-3-yl)-2,2,2-trifluoroethyl Trifluoromethanesulfonate

To a stirred solution of 1-(6-bromopyridin-3-yl)-2,2,2-trifluoroethan-1-ol (3.0 g, 16.0 mmol, CAS 1188477-81-7) in DCM (30 mL) at −40° C. was added trifluoromethanesulfonic anhydride (3.6 mL, 24.1 mmol) and the mixture was stirred at −40° C. for 1 h. The mixture was diluted with ice/water, then extracted with hexane. The organic layer was dried over Na₂SO₄, filtered and concentrated under reduced pressure to provide the title compound (3.5 g). LCMS (Method A): 387.9 [M+H]⁺.

### Intermediate 24: 1-(6-Bromopyridin-3-yl)-2,2,2-trifluoro-N-methylethan-1-amine

A solution of Intermediate 23 (3.0 g, 7.73 mmol) in methylamine in THF (2M, 30 mL) was stirred at 60° C. for 4 h. The mixture was concentrated under reduced pressure and the crude was purified by flash chromatography (silica gel, eluting 10-70% EtOAc in petroleum ether) to provide the title compound (2.5 g). LCMS (Method A): 269.2 [M+H]⁺.

### Intermediate 25: 1-(6-Bromopyridin-3-yl)-2,2,2-trifluoro-N-methylethan-1-amine Hydrochloride

To a stirred solution of Intermediate 24 (2.5 g, 9.29 mmol) in 1,4-dioxane (25 mL), was added 4M HCl in 1,4-dioxane (25 mL) at 0° C. and the mixture was stirred at RT for 2 h. The mixture was concentrated under reduced pressure. The crude product was triturated in diethyl ether and filtered to provide the title compound (2.5 g). LCMS (Method C): 269.0 [M+H]⁺.

### Intermediate 26: N-(1,1-Dimethyl-2,3-dihydro-1H-inden-2-yl)-5-(2,2,2-trifluoro-1-(methylamino)ethyl)pyridin-2-amine

To a stirred solution of Intermediate 25 (1.5 g, 4.91 mmol) and 1,1-dimethyl-2,3-dihydro-1H-inden-2-amine hydrochloride (1.32 g, 6.69 mmol, CAS 74413-86-8) in toluene (15 mL) was added sodium t-butoxide (2.14 g, 22.3 mmol) and the mixture was degassed with argon for 5 min. Pd₂(dba)₃ (0.51 g, 0.56 mmol) and X-Phos (0.53 g, 1.12 mmol) were added then the mixture was heated at 110° C. for 2 h under microwave irraditation. The mixture was poured into water and extracted with EtOAc. The organic layer was dried over Na₂SO₄, filtered and concentrated under reduced pressure. The crude product was purified by flash chromatography (silica gel, eluting 13-15% EtOAc in petroleum ether) to provide the title compound (0.45 g). LCMS (Method A): 350.3 [M+H]⁺.

### Intermediate 27: N-(1,1-Dimethyl-2,3-dihydro-1H-inden-2-yl)-5-(2,2,2-trifluoro-1-(methylamino)ethyl)pyridin-2-amine Hydrochloride

To a stirred solution of Intermediate 26 (0.45 g, 1.29 mmol) in 1,4-dioxane (2.5 mL), was added 4M HCl in 1,4-dioxane (2.5 mL) at 0° C. and the mixture was stirred at RT for 2 h. The mixture was concentrated under reduced pressure. The crude product was triturated in EtOAc and filtered to provide the title compound (0.40 g). LCMS (Method A): 350.3 [M+H]⁺.

### Intermediate 28: tert-Butyl (S)-3-((1-(4-bromophenyl)-2,2,2-trifluoroethyl)(methyl)carbamoyl)-3-methylazetidine-1-carboxylate

The title compound (0.31 g) was prepared in an analogous manner to Intermediate 4 from Intermediate 2 (1.70 g, 6.51 mmol), 1-(tert-butoxycarbonyl)-3-methylazetidine-3-carboxylic acid (1.40 g, 6.51 mmol, CAS 887591-62-0), POCl₃ (0.73 mL, 7.80 mmol) in pyridine (15 mL) at RT for 2 h. Purified by flash chromatography (silica gel, eluting 20-50% EtOAc in petroleum ether). LCMS (Method A): 409.3 [M−tBu+H]⁺.

### Intermediate 29: (S)—N-(1-(4-Bromophenyl)-2,2,2-trifluoroethyl)-N,3-dimethylazetidine-3-carboxamide Hydrochloride

To a stirred solution of Intermediate 28 (0.30 g, 0.64 mmol) in 1,4-dioxane (5 mL), was added 4M HCl in 1,4-dioxane (5 mL) at 0° C. and the mixture was stirred at 0° C. for 4 h. The mixture was concentrated under reduced pressure. The crude product was triturated in diethyl ether and filtered to provide the title compound (0.30 g). LCMS (Method A): 365.3 [M+H]⁺.

### Intermediate 30: (S)-1-Acetyl-N-(1-(4-bromophenyl)-2,2,2-trifluoroethyl)-N,3-dimethylazetidine-3-carboxamide

To a stirred mixture of Intermediate 29 (3.0 g, 7.73 mmol) in DCM (6 mL) was added triethylamine (0.32 mL, 2.25 mmol) and acetic anhydride (0.1 mL, 1.12 mmol) at 0° C. and stirred for 3 h. The mixture was poured into ice water, basified with saturated aqueous NaHCO₃ then extracted with EtOAc. The organic layer was dried over Na₂SO₄, filtered and concentrated under reduced pressure. The crude was purified by flash chromatography (silica gel, eluting 20-50% EtOAc in petroleum ether) to provide the title compound (0.40 g). LCMS (Method A): 409.3 [M+H]⁺.

### Intermediate 31: tert-Butyl (S)-3-((1-(4-bromophenyl)-2,2,2-trifluoroethyl)(methyl)carbamoyl)-3-fluoroazetidine-1-carboxylate

The title compound (1.38 g) was prepared in an analogous manner to Intermediate 4 from Intermediate 2 (2.20 g, 8.21 mmol), 1-(tert-butoxycarbonyl)-3-fluoroazetidine-3-carboxylic acid (1.80 g, 8.21 mmol, CAS 1126650-67-6), POCl₃ (1.88 mL, 20.1 mmol) in pyridine (12 mL) at RT for 30 min. Purified by flash chromatography (silica gel, eluting 10-15% EtOAc in petroleum ether). LCMS (Method A): 413.3 [M−tBu+H]⁺.

### Intermediate 32: (S)—N-(1-(4-Bromophenyl)-2,2,2-trifluoroethyl)-3-fluoro-N-methylazetidine-3-carboxamide Hydrochloride

To a stirred solution of Intermediate 31 (1.38 g, 2.94 mmol) in EtOAc (20 mL), was added 4M HCl in 1,4-dioxane (20 mL) at RT and the mixture was stirred at RT for 2 h. The mixture was concentrated under reduced pressure. The crude product was triturated in diethyl ether and filtered to provide the title compound (1.10 g). LCMS (Method A): 369.0 [M+H]⁺.

### Intermediate 33: (S)-1-Acetyl-N-(1-(4-bromophenyl)-2,2,2-trifluoroethyl)-3-fluoro-N-methylazetidine-3-carboxamide

To a stirred mixture of Intermediate 32 (1.10 g, 2.71 mmol) in DCM (8 mL) was added pyridine (8 mL), DMAP (0.03 g, 0.25 mmol) and acetic anhydride (0.56 g, 5.43 mmol) at RT and stirred for 2 h. The mixture was poured into ice water, basified with saturated aqueous NaHCO₃ then extracted with EtOAc. The organic layer was dried over Na₂SO₄, filtered and concentrated under reduced pressure. The crude was purified by flash chromatography (silica gel, eluting 40-50% EtOAc in petroleum ether) to provide the title compound (1.0 g). LCMS (Method A): 411.3 [M+H]⁺.

### Intermediate 34: (S)—N-(1-(4-Bromophenyl)-2,2,2-trifluoroethyl)-N-methyl-2-(2-oxopyrrolidin-1-yl)acetamide

The title compound (0.40 g) was prepared in an analogous manner to Intermediate 3 from Intermediate 2 (0.5 g, 1.87 mmol), DIPEA (1.30 mL, 7.46 mmol), T3P® (50% EtOAc, 5.93 mL, 9.33 mmol) and 2-(2-oxopyrrolidin-1-yl)acetic acid (0.32 g, 2.24 mmol, CAS 53934-76-2) in THF (5 mL) at 80° C. for 12 h. The crude product was purified by flash chromatography (silica gel, eluting 80% EtOAc in petroleum ether). LCMS (Method A): 393.3 [M+H]⁺.

### Intermediate 35: (S)—N-(1-(4-Bromophenyl)-2,2,2-trifluoroethyl)-N-methyl-3-(2-oxopyrrolidin-1-yl)propanamide

The title compound (0.35 g) was prepared in an analogous manner to Intermediate 3 from Intermediate 2 (0.4 g, 1.32 mmol), DIPEA (0.73 mL, 3.96 mmol), T3P® (50% EtOAc, 2.52 mL, 3.96 mmol) and 3-(2-oxopyrrolidin-1-yl)propanoic acid (0.31 g, 1.98 mmol, CAS 77191-38-9) in THF (10 mL) at 80° C. for 16 h. The crude product was purified by flash chromatography (silica gel, eluting 30-70% EtOAc in petroleum ether). LCMS (Method A): 407.3 [M+H]⁺.

### Intermediate 36: Ethyl (S)-5-((1-(4-bromophenyl)-2,2,2-trifluoroethyl)(methyl)amino)-5-oxopentanoate

The title compound (0.59 g) was prepared in an analogous manner to Intermediate 4 from Intermediate 2 (0.75 g, 2.46 mmol), 5-ethoxy-5-oxopentanoic acid (0.16 g, 9.85 mmol, CAS 1070-62-8), POCl₃ (0.90 mL, 9.85 mmol) in pyridine (7.5 mL) at RT for 1 h. Purified by flash chromatography (silica gel, eluting 10-15% EtOAc in petroleum ether). LCMS (Method A): 410.1 [M+H]⁺.

### Intermediate 37: Ethyl 5-(((1S)-1-(4-((1,1-dimethyl-2,3-dihydro-1H-inden-2-yl)amino)phenyl)-2,2,2-trifluoroethyl)(methyl)amino)-5-oxopentanoate

The title compound (0.24 g) was prepared in an analogous manner to Intermediate 11 from Intermediate 36 (0.5 g, 1.22 mmol), 1,1-dimethyl-2,3-dihydro-1H-inden-2-amine hydrochloride (0.24 g, 1.22 mmol, CAS 74413-86-8), Cs₂CO₃ (1.19 g, 3.66 mmol), tBuBrettphosPdG3 (104 mg, 0.12 mmol) and RuPhos (114 mg, 0.24 mmol) in 1,4-dioxane (4 mL) and DMF (1 mL) at 120° C. for 2 h. Purified by flash chromatography (silica gel, eluting 20-25% EtOAc in petroleum ether). LCMS (Method A): 491.3 [M+H]⁺.

### Intermediate 38: Ethyl (S)-4-((1-(4-bromophenyl)-2,2,2-trifluoroethyl)(methyl)amino)-4-oxobutanoate

The title compound (1.2 g) was prepared in an analogous manner to Intermediate 4 from Intermediate 2 (1.0 g, 3.30 mmol), 4-ethoxy-4-oxobutanoic acid (0.87 g, 6.60 mmol, CAS 1070-34-4), POCl₃ (0.63 mL, 6.60 mmol) in pyridine (10 mL) at RT for 2 h. Purified by flash chromatography (silica gel, eluting 70-100% EtOAc in petroleum ether). LCMS (Method A): 396.3 [M+H]⁺.

### Intermediate 39: Ethyl 4-(((1S)-1-(4-((1,1-dimethyl-2,3-dihydro-1H-inden-2-yl)amino)phenyl)-2,2,2-trifluoroethyl)(methyl)amino)-4-oxobutanoate

The title compound (0.25 g) was prepared in an analogous manner to Intermediate 11 from Intermediate 38 (0.8 g, 2.02 mmol), 1,1-dimethyl-2,3-dihydro-1H-inden-2-amine hydrochloride (0.40 g, 2.02 mmol, CAS 74413-86-8), Cs₂CO₃ (1.64 g, 5.05 mmol), tBuBrettphosPdG3 (0.34 g, 0.40 mmol) and RuPhos (0.37 g, 0.81 mmol) in 1,4-dioxane (8 mL) at 120° C. for 4 h. Purified by flash chromatography (silica gel, eluting 70-100% EtOAc in petroleum ether). LCMS (Method A): 477.5 [M+H]⁺.

### Intermediate 40: (S)-(1-(4-Bromophenyl)-2,2,2-trifluoroethyl)(methyl)carbamic Chloride

To a stirred solution of Intermediate 2 (1.50 g, 4.93 mmol) in DCM (15 mL) was added triphosgene (1.53 g, 5.17 mmol) portionwise at 0° C. and stirred for 30 min. Triethylamine (1.37 mL, 9.85 mmol) was added and stirred at RT for 2 h. The mixture was quenched with ice cold water, then extracted with DCM. The organic layer was dried over Na₂SO₄, filtered and concentrated under reduced pressure. The crude product was purified by flash chromatography (silica gel, eluting 20% EtOAc in petroleum ether) to provide the title compound (1.0 g). LCMS (Method A): 329.9 [M+H]⁺.

### Intermediate 41: tert-Butyl (S)-3-(((1-(4-bromophenyl)-2,2,2-trifluoroethyl) (methyl)carbamoyl)oxy)azetidine-1-carboxylate

To a stirred mixture of tert-butyl 3-hydroxyazetidine-1-carboxylate (0.63 g, 3.63 mmol, CAS 141699-55-0) in THF (15 mL) was added sodium hydride (60% in mineral oil, 0.24 g, 6.05 mmol) portionwise at 0° C. and stirred for 30 min at RT. Intermediate 40 (1.0 g, 3.03 mmol) was added as a solution in THF (10 mL) at 0° C. then the mixture was stirred at RT for 2 h. The mixture was poured into water then extracted with EtOAc. The organic layer was dried over Na₂SO₄, filtered and concentrated under reduced pressure. The crude was purified by flash chromatography (silica gel, eluting 20% EtOAc in petroleum ether) to provide the title compound (0.7 g). LCMS (Method A): 411.3 [M−tBu+H]⁺.

### Intermediate 42: (R)—N-(1-(4-Bromophenyl)ethyl)-N-methyltetrahydro-2H-thiopyran-4-carboxamide 1,1-dioxide

The title compound (1.0 g) was prepared in an analogous manner to Intermediate 3 from (R)-1-(4-bromophenyl)-N-methylethan-1-amine hydrochloride (1.2 g, 5.05 mmol, CAS 2366996-95-2), pyridine (1.22 mL, 15.2 mmol), T3P® (50% EtOAc, 15.2 mL, 50.5 mmol) and tetrahydro-2H-thiopyran-4-carboxylic acid 1,1-dioxide (0.90 g, 5.05 mmol, CAS 64096-87-3) at RT for 16 h. The crude product was purified by flash chromatography (silica gel, eluting 50-55% EtOAc in petroleum ether). LCMS (Method A): 374.0 [M+H]⁺.

### Intermediate 43: (S)—N-(1-(4-Bromophenyl)-2,2,2-trifluoroethyl)-N-methyl-1-(methylsulfonyl)azetidine-3-carboxamide

The title compound (0.45 g) was prepared in an analogous manner to Intermediate 4 from Intermediate 2 (0.50 g, 1.64 mmol), 1-(methylsulfonyl)azetidine-3-carboxylic acid (0.38 g, 2.13 mmol, CAS 1219828-27-9), POCl₃ (0.30 mL, 3.28 mmol) in pyridine (5 mL) at RT for 2 h. Purified by flash chromatography (silica gel, eluting 25-30% EtOAc in petroleum ether). LCMS (method A): 430.0 [M+H]⁺.

### Intermediate 44: tert-Butyl (S)-4-((1-(4-bromophenyl)-2,2,2-trifluoroethyl) (methyl)carbamoyl)piperidine-1-carboxylate

The title compound (2.1 g) was prepared in an analogous manner to Intermediate 3 from Intermediate 2 (2.0 g, 6.56 mmol), pyridine (2.11 mL, 26.3 mmol), T3P® (50% EtOAc, 29.3 mL, 45.97 mmol) and 1-(tert-butoxycarbonyl)piperidine-4-carboxylic acid (0.31 g, 1.98 mmol, CAS 84358-13-4) at RT for 12 h. The crude product was purified by flash chromatography (silica gel, eluting 30% EtOAc in petroleum ether). LCMS (Method A): 425.1 [M−tBu+H]⁺.

### Intermediate 45: tert-Butyl (S)-4-((1-(4-((2,3-dihydro-1H-inden-2-yl)amino)phenyl)-2,2,2-trifluoroethyl)(methyl)carbamoyl)piperidine-1-carboxylate

The title compound (1.17 g) was prepared in an analogous manner to Intermediate 11 from Intermediate 44 (2.1 g, 4.38 mmol), 2,3-dihydro-1H-inden-2-amine hydrochloride (0.74 g, 4.38 mmol, CAS 2975-41-9), Cs₂CO₃ (5.70 g, 17.5 mmol), tBuBrettphosPdG3 (0.37 g, 0.43 mmol) and RuPhos (0.40 g, 0.87 mmol) in 1,4-dioxane (42 mL) at 130° C. for 2 h. Purified by flash chromatography (silica gel, eluting 50% EtOAc in petroleum ether). LCMS (Method A): 532.3 [M+H]⁺.

### Intermediate 46: 1-(5-Bromopyridin-2-yl)-2,2,2-trifluoro-N-methylethan-1-amine

The title compound (3.9 g) was prepared in an analogous manner to Intermediate 24 from 1-(5-bromopyridin-2-yl)-2,2,2-trifluoroethyl trifluoromethanesulfonate (7.0 g, 18.0 mmol, CAS 1374038-21-7) and methylamine in THF (2M, 35 mL) at 60° C. for 2 h. The crude was purified by flash chromatography (silica gel, eluting 10-15% EtOAc in petroleum ether). LCMS (Method A): 269.1 [M+H]⁺.

### Intermediate 47: 1-(5-Bromopyridin-2-yl)-2,2,2-trifluoro-N-methylethan-1-amine Hydrochloride

To a stirred solution of Intermediate 46 (2.0 g, 7.43 mmol) in EtOAc (10 mL), was added 4M HCl in 1,4-dioxane (10 mL) at RT and the mixture was stirred at RT for 30 min. The mixture was concentrated under reduced pressure to provide the title compound (2.0 g). LCMS (Method A): 269.1 [M+H]⁺.

### Intermediate 48: N-(1-(5-Bromopyridin-2-yl)-2,2,2-trifluoroethyl)-N-methyltetrahydro-2H-thiopyran-4-carboxamide 1,1-dioxide

The title compound (5.8 g) was prepared in an analogous manner to Intermediate 3 from Intermediate 47 (6.0 g, 19.8 mmol), pyridine (60 mL), T3P® (50% EtOAc, 31.5 g, 99.0 mmol) and tetrahydro-2H-thiopyran-4-carboxylic acid 1,1-dioxide (4.30 g, 23.8 mmol, CAS 64096-87-3) at 60° C. for 5 h. The crude product was purified by flash chromatography (silica gel, eluting 35-40% EtOAc in petroleum ether). LCMS (Method A): 431.0 [M+H]⁺.

### Intermediate 49: tert-Butyl (S)-3-((1-(4-bromophenyl)-2,2,2-trifluoroethyl) (methyl)carbamoyl)azetidine-1-carboxylate

The title compound (2.8 g) was prepared in an analogous manner to Intermediate 3 from Intermediate 2 (2.0 g, 6.56 mmol), pyridine (2.11 mL, 26.3 mmol), T3P® (50% EtOAc, 29.3 mL, 46.0 mmol) and 1-(tert-butoxycarbonyl)azetidine-3-carboxylic acid (1.98 g, 9.85 mmol, CAS 142253-55-2) at RT for 12 h. The crude product was purified by flash chromatography (silica gel, eluting 30% EtOAc in hexane). LCMS (Method A): 397.0 [M−tBu+H]⁺.

### Intermediate 50: tert-Butyl 3-(((1S)-1-(4-((1,1-dimethyl-2,3-dihydro-1H-inden-2-yl)amino)phenyl)-2,2,2-trifluoroethyl)(methyl)carbamoyl)azetidine-1-carboxylate

The title compound (0.45 g) was prepared in an analogous manner to Intermediate 11 from Intermediate 49 (0.9 g, 1.99 mmol), 1,1-dimethyl-2,3-dihydro-1H-inden-2-amine hydrochloride (0.39 g, 1.99 mmol, CAS 74413-86-8), Cs₂CO₃ (2.59 g, 7.97 mmol), tBuBrettphosPdG3 (0.20 g, 0.24 mmol) and RuPhos (0.23 g, 0.49 mmol) in 1,4-dioxane (27 mL) at 160° C. for 1 h. Purified by flash chromatography (silica gel, eluting 40% EtOAc in petroleum ether). LCMS (Method A): 532.3 [M+H]⁺.

### Intermediate 51: (S)—N-(1-(4-Bromophenyl)-2,2,2-trifluoroethyl)tetrahydro-2H-thiopyran-4-carboxamide 1,1-dioxide

The title compound (1.9 g) was prepared in an analogous manner to Intermediate 3 from Intermediate 2 (1.75 g, 6.02 mmol), pyridine (1.46 mL, 18.1 mmol), T3P® (50% EtOAc, 38.3 g, 60.2 mmol) and tetrahydro-2H-thiopyran-4-carboxylic acid 1,1-dioxide (1.07 g, 6.02 mmol, CAS 64096-87-3) at RT for 16 h. The crude product was purified by flash chromatography (silica gel, eluting 40% EtOAc in hexane). LCMS (Method A): 416.2 [M+H]⁺.

### Intermediate 52: tert-Butyl 3-((1-(5-bromopyridin-2-yl)-2,2,2-trifluoroethyl)(methyl)carbamoyl)azetidine-1-carboxylate

The title compound (1.1 g) was prepared in an analogous manner to Intermediate 3 from Intermediate 47 (1.0 g, 3.27 mmol), pyridine (10 mL), T3P® (50% EtOAc, 10 mL, 15.7 mmol) and 1-(tert-butoxycarbonyl)azetidine-3-carboxylic acid (0.79 g, 3.92 mmol, CAS 142253-55-2) in THF (10 mL) at RT for 16 h. The crude product was purified by flash chromatography (silica gel, eluting 25% EtOAc in petroleum ether). LCMS (Method A): 396.0 [M−tBu+H]⁺.

### Intermediate 53: tert-Butyl 3-((1-(5-((1,1-dimethyl-2,3-dihydro-1H-inden-2-yl)amino)pyridin-2-yl)-2,2,2-trifluoroethyl)(methyl)carbamoyl)azetidine-1-carboxylate

The title compound (0.6 g) was prepared in an analogous manner to Intermediate 11 from Intermediate 52 (1.0 g, 1.10 mmol), 1,1-dimethyl-2,3-dihydro-1H-inden-2-amine hydrochloride (0.21 g, 1.10 mmol, CAS 74413-86-8), Cs₂CO₃ (1.08 g, 3.31 mmol), and RuPhosPdG3 (0.09 g, 0.11 mmol) in toluene (10 mL) at 110° C. for 16 h. Purified by flash chromatography (silica gel, eluting 40% EtOAc in petroleum ether). LCMS (Method A): 533.5 [M+H]⁺.

### Intermediate 54: tert-Butyl 3-((1-(6-((1,1-dimethyl-2,3-dihydro-1H-inden-2-yl)amino)pyridin-3-yl)-2,2,2-trifluoroethyl)(methyl)carbamoyl)azetidine-1-carboxylate

The title compound (0.3 g) was prepared in an analogous manner to Intermediate 3 from Intermediate 26 (0.5 g, 1.43 mmol), pyridine (5 mL), T3P® (50% EtOAc, 4.5 mL, 7.16 mmol) and 1-(tert-butoxycarbonyl)azetidine-3-carboxylic acid (0.58 g, 2.86 mmol, CAS 142253-55-2) in THF (5 mL) at RT for 16 h. The crude product was purified by flash chromatography (silica gel, eluting 70% EtOAc in petroleum ether). LCMS (Method A): 533.5 [M+H]⁺.

### Intermediate 55: N-(1-(5-Bromopyridin-2-yl)-2,2,2-trifluoroethyl)-N-methylpivalamide

To a stirred mixture of Intermediate 47 (1.0 g, 3.27 mmol) in DCM (15 mL) was added triethylamine (2.28 mL, 16.4 mmol) at 0° C. and stirred for 5 min. Trimethylacetyl chloride (1.2 mL, 9.82 mmol) was added, the reaction tube was sealed, then the mixture was stirred at 40° C. for 8 h. The mixture was poured into water then extracted with DCM. The organic layer was dried over Na₂SO₄, filtered and concentrated under reduced pressure. The crude product was purified by flash chromatography (neutral alumina, eluting 5% EtOAc in petroleum ether) to provide the title compound (1.1 g). LCMS (Method A): 353.2 [M+H]⁺.

### Intermediate 56: N-(1-(5-((1,1-Dimethyl-2,3-dihydro-1H-inden-2-yl)amino)pyridin-2-yl)-2,2,2-trifluoroethyl)-N-methylpivalamide

The title compound (0.7 g) was prepared in an analogous manner to Intermediate 11 from Intermediate 55 (1.1 g, 3.12 mmol), 1,1-dimethyl-2,3-dihydro-1H-inden-2-amine hydrochloride (0.62 g, 3.12 mmol, CAS 74413-86-8), Cs₂CO₃ (3.04 g, 9.34 mmol), and RuPhosPdG3 (0.26 g, 0.31 mmol) in toluene (10 mL) at 110° C. for 16 h. Purified by flash chromatography (silica gel, eluting 20% EtOAc in petroleum ether). LCMS (Method A): 434.4 [M+H]⁺.

### Intermediate 57: N-(1,1-Dimethyl-2,3-dihydro-1H-inden-2-yl)-6-(2,2,2-trifluoro-1-(methylamino)ethyl)pyridin-3-amine Hydrochloride

Intermediate 56 (0.50 g, 7.43 mmol) was suspended in 6M HCl (10 mL) and the mixture was stirred at 110° C. for 16 h. The mixture was diluted with water and washed with diethyl ether. The aqueous layer was basified with saturated aqueous NaHCO₃ then extracted with EtOAc. The organic was dried over Na₂SO₄, filtered and concentrated under reduced pressure. The residue was diluted with EtOAc, 2M HCl in diethyl ether was added at 0° C. then concentrated under reduced pressure to provide the title compound (0.35 g). LCMS (Method A): 350.3 [M+H]⁺.

### Intermediate 58: (S)-1-(5-Bromopyridin-2-yl)-2,2,2-trifluoro-N-methylethan-1-amine

The title compound (3.86 g) was prepared from Intermediate 46 (9.0 g, 33.5 mmol) by preparative SFC (LUX AMYLOSE-i3, 30×250 mm×5 μm, temp 30° C., Back Pressure 100 bar, flow rate: 95 g/min, 93% CO₂ with 7% MeCN modifier). LCMS (Method A): 269.1 [M+H]⁺, 98% ee by chiral SFC.

### Intermediate 59: (S)—N-(1-(5-Bromopyridin-2-yl)-2,2,2-trifluoroethyl)-N-methylpivalamide

The title compound (1.0 g) was prepared in an analogous manner to Intermediate 55 from Intermediate 58 (1.0 g, 3.72 mmol), trimethylacetyl chloride (1.37 mL, 11.2 mmol), triethylamine (2.68 mL, 18.6 mmol) in DCM (10 mL) at RT for 16 h. Purified by flash chromatography (silica gel, eluting 2-4% EtOAc in petroleum ether). LCMS (Method A): 353.2 [M+H]⁺.

### Intermediate 60: N-((1S)-1-(5-((1,1-Dimethyl-2,3-dihydro-1H-inden-2-yl)amino)pyridin-2-yl)-2,2,2-trifluoroethyl)-N-methylpivalamide

The title compound (0.65 g) was prepared in an analogous manner to Intermediate 11 from Intermediate 59 (1.0 g, 2.83 mmol), 1,1-dimethyl-2,3-dihydro-1H-inden-2-amine hydrochloride (0.84 g, 4.25 mmol, CAS 74413-86-8), Cs₂CO₃ (2.77 g, 8.49 mmol), and RuPhosPdG3 (0.24 g, 0.28 mmol) in toluene (10 mL) at 110° C. for 16 h. Purified by flash chromatography (silica gel, eluting 4-8% EtOAc in petroleum ether). LCMS (Method A): 434.4 [M+H]⁺.

### Intermediate 61: N-(1,1-Dimethyl-2,3-dihydro-1H-inden-2-yl)-6-((S)-2,2,2-trifluoro-1-(methylamino)ethyl)pyridin-3-amine

To a stirred solution of Intermediate 60 (0.65 g, 1.50 mmol) in THF (6 mL) at −15° C. was added LiAlH₄ (2M in THF, 1.5 mL, 3.0 mmol) slowly so that the temperature did not exceed 0° C. The mixture was stirred at −10° C. for 30 min. The mixture was quenched with ice cold aqueous NH₄Cl and then extracted with EtOAc. The organic layer was dried over Na₂SO₄, filtered and concentrated under reduced pressure. The crude product was purified by flash chromatography (silica gel, eluting 15-20% EtOAc in petroleum ether) to provide the title compound (0.25 g). LCMS (Method A): 350.3 [M+H]⁺.

### Intermediate 62: (S)-1-(6-Bromopyridin-3-yl)-2,2,2-trifluoro-N-methylethan-1-amine

The title compound (8.5 g) was prepared from Intermediate 24 (28 g) by preparative SFC (LUX Cellulose-i-A3, 30×250 mm×5 μm, temp 30° C., Back Pressure 100 bar, flow rate: 95 g/min, 90% CO₂ with 7% MeOH modifier). LCMS (Method A): 269.0 [M+H]⁺, 95.7% ee by chiral SFC.

### Intermediate 63: N-(1,1-Dimethyl-2,3-dihydro-1H-inden-2-yl)-5-((S)-2,2,2-trifluoro-1-(methylamino)ethyl)pyridin-2-amine

The title compound (0.55 g) was prepared in an analogous manner to Intermediate 26 from Intermediate 62 (1.0 g, 3.71 mmol), 1,1-dimethyl-2,3-dihydro-1H-inden-2-amine hydrochloride (0.73 g, 3.71 mmol, CAS 74413-86-8), sodium t-butoxide (1.07 g, 11.2 mmol), Pd₂(dba)₃ (0.34 g, 0.37 mmol) and X-Phos (0.35 g, 0.74 mmol) in toluene (10 mL) at 110° C. for 2 h under microwave irradiation. Purified by flash chromatography (silica gel, eluting 25% EtOAc in petroleum ether). LCMS (Method A): 350.3 [M+H]⁺.

### Intermediate 64: N-(1,1-Dimethyl-2,3-dihydro-1H-inden-2-yl)-5-((S)-2,2,2-trifluoro-1-(methylamino)ethyl)pyridin-2-amine Hydrochloride

To a stirred solution of Intermediate 63 (0.80 g, 2.29 mmol) in EtOAc (8 mL), was added 4M HCl in 1,4-dioxane (4 mL) at 0° C. and the mixture was stirred at RT for 2 h. The mixture was concentrated under reduced pressure. The crude product was triturated in n-pentane and filtered to provide the title compound (0.60 g). LCMS (Method C): 350.5 [M+H]⁺.

### Intermediate 65: tert-Butyl 3-(((1S)-1-(5-((1,1-dimethyl-2,3-dihydro-1H-inden-2-yl)amino)pyridin-2-yl)-2,2,2-trifluoroethyl)(methyl)carbamoyl)piperidine-1-carboxylate

The title compound (0.55 g) was prepared in an analogous manner to Intermediate 3 from Intermediate 61 (0.30 g, 0.85 mmol), pyridine (1.5 mL), T3P® (50% EtOAc, 3.8 mL, 6.01 mmol) and 1-(tert-butoxycarbonyl)piperidine-3-carboxylic acid (0.40 g, 1.71 mmol, CAS 84358-12-3) at RT for 16 h. The crude product was purified by flash chromatography (silica gel, eluting 20-25% EtOAc in petroleum ether). LCMS (Method A): 561.5 [M+H]⁺.

### Intermediate 66: Methyl 3-((2,4-dimethoxybenzyl)amino)-2-hydroxypropanoate

To a stirred solution of methyl 3-amino-2-hydroxypropanoate hydrochloride (9.0 g, 57.9 mmol, CAS 186393-00-0) in methanol (90 mL) at 0° C. was added triethylamine (16 mL, 115 mmol) and 2,4-dimethoxybenzaldehyde (10.6 g, 63.6 mmol) and then the mixture was heated at 60° C. for 16 h. The mixture was cooled to 0° C. then sodium borohydride (4.38 g, 116 mmol) was added then the mixture stirred at RT for 3 h. The mixture was quenched with saturated aqueous NH₄Cl and then extracted with EtOAc. The organic layer was concentrated under reduced pressure. The crude product was purified by flash chromatography (silica gel, eluting EtOAc with 0.1% triethylamine) to provide the title compound (5.0 g). LCMS (Method C): 270.3 [M+H]⁺.

### Intermediate 67: 4-(2,4-Dimethoxybenzyl)-5-oxomorpholine-2-carboxylic Acid

To a stirred solution of Intermediate 66 (2.5 g, 9.28 mmol) in 3.7 M NaOH (25 mL) at 0° C. was added chloroacetyl chloride (1.25 g, 11.1 mmol) and the mixture was stirred at RT for 30 min. The mixture was adjusted to pH 13 by addition of 50% aqueous NaOH (10 mL) then stirred at RT for 16 h. The mixture was quenched with 2M aqueous HCl until pH 2 and then extracted with EtOAc. The organic layer was concentrated under reduced pressure. The crude product was purified by flash chromatography (silica gel, eluting 10% MeOH in DCM with 0.1% triethylamine) to provide the title compound (0.1 g). LCMS (Method C): 296.4 [M+H]⁺.

### Intermediate 68: 5-Oxomorpholine-2-carboxylic Acid

A stirred solution of Intermediate 67 (0.2 g, 0.54 mmol) in TFA (1.0 mL) was heated at 70° C. for 16 h. The mixture was concentrated under reduced pressure to provide the title compound (0.09 g). LCMS (Method C): 146.1 [M+H]⁺.

### Intermediate 69: N-(1-(5-((1,1-Dimethyl-2,3-dihydro-1H-inden-2-yl)amino)pyridin-2-yl)-2,2,2-trifluoroethyl)-N-methylazetidine-3-carboxamide

To a stirred mixture of Intermediate 53 (0.60 g, 1.12 mmol) in DCM (12 mL) at 0° C. was added TFA (3 mL) and stirred at RT for 16 h. The mixture was diluted with saturated aqueous NaHCO₃ and extracted with DCM. The organic layer was dried over Na₂SO₄, filtered and concentrated under reduced pressure. The crude product was purified by preparative HPLC (XBridge C18, 19×250 mm×5 μm, flow rate: 18 mL/min, 10 mM aqueous NH₄HCO₃ with MeCN 10% to 50% over 7 min, held at 50% for 8 min then ramped to 98% over 0.5 min and held for 3.5 min) to provide the title compound (0.11 g). LCMS (Method G): 431.6 [M−H]⁻.

### Intermediate 70: N-(1-(6-((1,1-dimethyl-2,3-dihydro-1H-inden-2-yl)amino)pyridin-3-yl)-2,2,2-trifluoroethyl)-N-methylazetidine-3-carboxamide

To a stirred mixture of Intermediate 54 (0.35 g, 0.66 mmol) in DCM (3.5 mL) at 0° C. was added TFA (1.75 mL) and stirred at RT for 2 h. The mixture was concentrated under reduced pressure then diluted with saturated aqueous NaHCO₃ and extracted with DCM. The organic layer was dried over Na₂SO₄, filtered and concentrated under reduced pressure. The crude product was purified by preparative HPLC (XBridge C18, 19×250 mm×5 μm, flow rate: 18 mL/min, 10 mM aqueous NH₄HCO₃ with MeCN 10% to 50% over 7 min, held at 50% for 8 min then ramped to 98% over 0.5 min and held for 3.5 min) to provide the title compound (0.17 g). LCMS (Method G): 431.6 [M−H]⁻.

### Intermediate 71: N-((1S)-1-(5-((1,1-Dimethyl-2,3-dihydro-1H-inden-2-yl)amino)pyridin-2-yl)-2,2,2-trifluoroethyl)-N-methylpiperidine-3-carboxamide

To a stirred solution of Intermediate 65 (0.55 g, 0.98 mmol) in DCM (5.5 mL) was added TFA (0.44 mL, 3.92 mmol) at 0° C. then stirred for RT 16 h. The mixture was concentrated under reduced pressure then diluted with DCM and washed with saturated aqueous NaHCO₃, dried over Na₂SO₄, filtered and concentrated under reduced pressure. Purified by preparative HPLC (X-Select C18, 25×250 mm×5 μm, flow rate: 18 mL/min, 10 mM aqueous NH₄HCO₃ with MeCN 45% to 70% over 13 min, ramped to 98% over 0.1 min and held for 4.9 min) to provide the title compound (0.13 g). LCMS (Method C): 461.5 [M+H]⁺.

### Intermediate 72: tert-Butyl 4-(((1S)-1-(5-((1,1-dimethyl-2,3-dihydro-1H-inden-2-yl)amino)pyridin-2-yl)-2,2,2-trifluoroethyl)(methyl)carbamoyl)piperidine-1-carboxylate

The title compound (0.15 g) was prepared in an analogous manner to Intermediate 3 from Intermediate 61 (0.10 g, 0.28 mmol), pyridine (0.5 mL), T3P® (50% EtOAc, 0.64 g, 2.00 mmol) and 1-(tert-butoxycarbonyl)piperidine-4-carboxylic acid (0.20 g, 0.85 mmol, CAS 84358-13-4) at RT for 16 h. The crude product was purified by flash chromatography (silica gel, eluting 25% EtOAc in petroleum ether). LCMS (Method C): 561.6 [M+H]⁺.

### Intermediate 73: tert-Butyl 4-(((1S)-1-(6-((1,1-dimethyl-2,3-dihydro-1H-inden-2-yl)amino)pyridin-3-yl)-2,2,2-trifluoroethyl)(methyl)carbamoyl)piperidine-1-carboxylate

The title compound (0.10 g) was prepared in an analogous manner to Intermediate 3 from Intermediate 64 (0.30 g, 0.78 mmol), pyridine (1.5 mL), T3P® (50% EtOAc, 2.5 mL, 3.93 mmol) and 1-(tert-butoxycarbonyl)piperidine-4-carboxylic acid (0.54 g, 2.34 mmol, CAS 84358-13-4) at RT for 16 h. The crude product was purified by flash chromatography (silica gel, eluting 70% EtOAc in petroleum ether). LCMS (Method A): 561.5 [M+H]⁺.

### Intermediate 74: 5-Chloro-3,3-dimethyl-2,3-dihydro-1H-inden-1-one/6-Chloro-3,3-dimethyl-2,3-dihydro-1H-inden-1-one

To a stirred solution of 3-methyl-2-butenoic acid (15 g, 0.15 mol, CAS 541-47-9) in chlorobenzene (80 mL) at 0° C. was added aluminium chloride (50 g, 0.45 mol) portionwise then the mixture was stirred at 80° C. for 12 h. The mixture was cooled then quenched with cold 5 M aqueous HCl and then extracted with EtOAc. The organic layer was dried over Na₂SO₄, filtered and concentrated under reduced pressure. The crude product was purified by flash chromatography (silica gel, eluting 10-20% EtOAc in petroleum ether) to provide the title compounds as a mixture (19 g). LCMS (Method A): 195.2 [M+H]⁺.

### Intermediate 75: 5-Chloro-3,3-dimethyl-2,3-dihydro-1H-inden-1-ol/6-Chloro-3,3-dimethyl-2,3-dihydro-1H-inden-1-ol

To a stirred solution of Intermediate 74 (10 g, 51.4 mmol) in ethanol (100 mL) at 0° C. was added sodium borohydride (3.89 g, 103 mmol) portionwise then the mixture was stirred at RT for 1 h. The mixture was cooled then quenched with saturated aqueous NH₄Cl and then extracted with EtOAc. The organic layer was dried over Na₂SO₄, filtered and concentrated under reduced pressure to provide the title compounds as a mixture (8.89 g). LCMS (Method A): 179.2 [M−18+H]⁺.

### Intermediate 76: 6-Chloro-1,1-dimethyl-1H-indene/5-Chloro-1,1-dimethyl-1H-indene

To a stirred solution of Intermediate 75 (8.7 g, 44.2 mmol) in toluene (80 mL) at 0° C. was added pTSA (4.21 g, 22.1 mmol) then the mixture was stirred at 120° C. for 1 h. The mixture was cooled then diluted with water and extracted with EtOAc. The organic layer was dried over Na₂SO₄, filtered and concentrated under reduced pressure. The crude product was purified by flash chromatography (silica gel, eluting 1-5% EtOAc in petroleum ether) to provide the title compounds as a mixture (5.0 g). ¹H NMR (400 MHz; CDCl₃) δ: 7.11-7.29 (m, 6H), 6.55 (t, 2H), 6.41-6.44 (m, 1H), 6.35 (d, 1H), 1.29 (d, 12H)—total for both compounds.

### Intermediate 77: 6-Chloro-1,1-dimethyl-1,3-dihydro-2H-inden-2-one/5-Chloro-1,1-dimethyl-1,3-dihydro-2H-inden-2-one

To a stirred solution of Intermediate 76 (2.0 g, 11.2 mmol) in acetone/t-butanol/water (5:1:1, 14 mL) at 0° C. was added osmium tetroxide (4% in water, 2.8 mL, 0.45 mmol) and NMMO (2.27 g, 16.8 mmol) then the mixture was stirred at RT for 16 h. The mixture was then diluted with water and extracted with EtOAc. The organic layer was dried over Na₂SO₄, filtered and concentrated under reduced pressure. The crude product was purified by flash chromatography (silica gel, eluting 8-15% EtOAc in petroleum ether). The residue was dissolved in benzene (10 mL) and pTSA (0.29 g, 1.5 mmol) was added and the mixture stirred at 80° C. for 1 h. The mixture was diluted with water and extracted with EtOAc. The organic layer was dried over Na₂SO₄, filtered and concentrated under reduced pressure. The crude product was purified by flash chromatography (silica gel, eluting 1-5% EtOAc in petroleum ether) to provide the title compounds as a mixture (1.0 g). LCMS (Method A): 193.2 [M−H]⁻.

### Intermediate 78a: 6-Chloro-1,1-dimethyl-2,3-dihydro-1H-inden-2-amine Hydrochloride

### Intermediate 78b: 5-Chloro-1,1-dimethyl-2,3-dihydro-1H-inden-2-amine Hydrochloride

To a stirred solution of Intermediate 77 (0.2 g, 1.03 mmol) in ethanol (10 mL) was added ammonium acetate (1.18 g, 15.3 mmol) and sodium cyanoborohydride (77 mg, 1.23 mmol) then the mixture was stirred at 100° C. for 1 h. The mixture was cooled then 4M HCl in 1,4-dioxane was added and then concentrated under reduced pressure. The residue was dissolved EtOAc and washed with saturated aqueous NaHCO₃. The organic layer was treated with 4M HCl in 1,4-dioxane, dried over Na₂SO₄, filtered and concentrated under reduced pressure to provide the title compounds as a mixture (1.0 g). LCMS (Method A): 196.2 [M+H]⁺.

### Intermediate 79a/Intermediate 79b: tert-Butyl 6-(((1S)-1-(5-((1,1-dimethyl-2,3-dihydro-1H-inden-2-yl)amino)pyridin-2-yl)-2,2,2-trifluoroethyl)(methyl)carbamoyl)-1-azaspiro[3.3]heptane-1-carboxylate

Prepared in an analogous manner to Intermediate 3 from Intermediate 61 (0.62 g, 1.77 mmol), pyridine (3.1 mL), T3P® (50% EtOAc, 8 mL, 12.4 mmol) and 1-(tert-butoxycarbonyl)-1-azaspiro[3.3]heptane-6-carboxylic acid (0.64 g, 2.66 mmol, CAS 1374659-11-6) at RT for 16 h. The crude product was purified by flash chromatography (silica gel, eluting 20-50% EtOAc in petroleum ether) to provide Intermediate 79a (0.57 g, diastereomer 1) and Intermediate 79b (0.38 g, diastereomer 2). 79a LCMS (Method A): 573.6 [M+H]⁺; 79b LCMS (Method A): 573.5 [M+H]⁺.

### Intermediate 80: (S)-1,1-Dimethyl-2,3-dihydro-1H-inden-2-amine Hydrochloride

### Intermediate 114: (R)-1,1-Dimethyl-2,3-dihydro-1H-inden-2-amine Hydrochloride

1,1-Dimethyl-2,3-dihydro-1H-inden-2-amine hydrochloride (3.8 g, 17.9 mmol, CAS 74413-86-8) was added to saturated aqueous NaHCO₃ and extracted with DCM containing 10% MeOH. The organic layer was dried over MgSO₄, filtered and concentrated under reduced pressure. Chiral purification was carried out by preparative SFC (Chiralpak®-IG, 30×250 mm×5 μm, temp 30° C., Back Pressure 100 bar, flow rate: 60 g/min, 50% CO₂ with 50% (MeCN with 0.2% diethylamine) modifier). After drying the separated peaks, 4M HCl in diethyl ether was added then concentrated to provide Intermediate 80 (1.15 g) as Peak 2 and Intermediate 114 (1.0 g) as Peak 1. Intermediate 80: ¹H NMR (400 MHz; DMSO-d₆) δ: 8.44 (br s, 3H), 7.26-7.17 (m, 4H), 3.55 (dd, 1H), 3.24 (dd, 1H), 2.99 (dd, 1H), 1.36 (s, 3H), 1.18 (s, 3H). Intermediate 114: ¹H NMR (400 MHz; DMSO-d₆) δ: 8.12 (br s, 3H), 7.25-7.16 (m, 4H), 3.51 (dd, 1H), 3.21 (dd, 1H), 2.98 (dd, 1H), 1.36 (s, 3H), 1.17 (s, 3H).

### Intermediate 81: N-((1S)-1-(5-(((S)-1,1-Dimethyl-2,3-dihydro-1H-inden-2-yl)amino)pyridin-2-yl)-2,2,2-trifluoroethyl)-N-methylpivalamide

The title compound (1.58 g) was prepared in an analogous manner to Intermediate 11 from Intermediate 59 (1.6 g, 4.53 mmol), Intermediate 80 (1.16 g, 5.89 mmol), Cs₂CO₃ (4.43 g, 3.0 mmol), and RuPhosPdG3 (0.55 g, 0.14 mmol) in toluene (22.7 mL) at 110° C. for 16 h. Purified by automated column chromatography (silica gel 120 g, eluting 0-40% EtOAc in hexane). ¹H NMR (300 MHz; DMSO-d₆) δ: 8.15 (d, 1H), 7.23-7.13 (m, 5H), 7.07 (d, 1H), 6.45 (dd, 1H), 6.21 (d, 1H), 4.10-3.99 (m, 1H), 3.20 (dd, 1H), 3.02 (s, 3H), 2.75 (dd, 1H), 1.33 (s, 3H), 1.25 (s, 9H), 1.10 (s, 3H).

### Intermediate 82: N—((S)-1,1-Dimethyl-2,3-dihydro-1H-inden-2-yl)-6-((S)-2,2,2-trifluoro-1-(methylamino)ethyl)pyridin-3-amine

The title compound (0.10 g) was prepared in an analogous manner to Intermediate 61 from Intermediate 81 (0.20 g, 0.46 mmol) and LiAlH₄ (1M in THF, 0.92 mL, 0.92 mmol) in THF (12.7 mL) at −13° C. for 30 min. The crude product was purified by automated column chromatography (silica gel, 40 g, eluting 0-50% EtOAc in hexane). LCMS (Method J): 350.2 [M+H]⁺.

### Intermediate 83: tert-Butyl (S)-2-(((S)-1-(5-(((S)-1,1-dimethyl-2,3-dihydro-1H-inden-2-yl)amino)pyridin-2-yl)-2,2,2-trifluoroethyl)(methyl)carbamoyl)morpholine-4-carboxylate

The title compound (0.10 g) was prepared in an analogous manner to Intermediate 3 from Intermediate 82 (0.20 g, 0.57 mmol), pyridine (1.9 mL), T3P® (50% EtOAc, 1.05 mL, 1.72 mmol) and (2S)-4-tert-butoxycarbonylmorpholine-2-carboxylic acid (0.54 g, 2.34 mmol, CAS 868689-63-8) at RT for 16 h. The crude product was purified by automated column chromatography (silica gel 40 g, eluting 5-50% EtOAc in hexanes). LCMS (Method J): 563.4 [M+H]⁺.

### Intermediate 84: tert-Butyl (S)-3-(((S)-1-(5-(((S)-1,1-dimethyl-2,3-dihydro-1H-inden-2-yl)amino)pyridin-2-yl)-2,2,2-trifluoroethyl)(methyl)carbamoyl)pyrrolidine-1-carboxylate

The title compound (0.19 g) was prepared in an analogous manner to Intermediate 3 from Intermediate 82 (0.15 g, 0.43 mmol), pyridine (2.1 mL), T3P® (50% in MeTHF, 0.80 mL, 1.3 mmol) and (3S)-1-tert-butoxycarbonylpyrrolidine-3-carboxylic acid (0.14 g, 0.64 mmol, CAS 140148-70-5) at RT for 16 h. The crude product was purified by prep-TLC (eluting 24:1 DCM/MeOH). LCMS (Method K): 547.1 [M+H]⁺.

### Intermediate 85: tert-Butyl ((1S,3R)-3-(((S)-1-(5-(((S)-1,1-dimethyl-2,3-dihydro-1H-inden-2-yl)amino)pyridin-2-yl)-2,2,2-trifluoroethyl)(methyl)carbamoyl)cyclopentyl)carbamate

The title compound (0.19 g) was prepared in an analogous manner to Intermediate 3 from Intermediate 82 (0.10 g, 0.29 mmol), pyridine (4.3 mL), T3P® (50% in MeTHF, 0.53 mL, 0.87 mmol) and (1R,3S)-3-(tert-butoxycarbonylamino)cyclopentane carboxylic acid (72 mg, 0.32 mmol, CAS 161660-94-2) at RT for 16 h. LCMS (Method K): 561.3 [M+H]⁺.

### Intermediate 86: (1R,3S)-3-Amino-N—((S)-1-(5-(((S)-1,1-dimethyl-2,3-dihydro-1H-inden-2-yl)amino)pyridin-2-yl)-2,2,2-trifluoroethyl)-N-methylcyclopentane-1-carboxamide TFA Salt

A solution of Intermediate 85 (0.14 g, 0.24 mmol) in TFA (0.2 mL) and DCM (1.5 mL) was stirred at RT for 1 h. The mixture was poured into water and extracted with EtOAc. The organics were dried over Na₂SO₄, filtered and concentrated under reduced pressure to provide the title compound (0.09 g). LCMS (Method K): 461.2 [M+H]⁺.

### Intermediate 87: tert-Butyl 6-(((S)-1-(5-(((S)-1,1-dimethyl-2,3-dihydro-1H-inden-2-yl)amino)pyridin-2-yl)-2,2,2-trifluoroethyl)(methyl)carbamoyl)-2-azaspiro[3.3]heptane-2-carboxylate

The title compound (0.23 g) was prepared in an analogous manner to Intermediate 3 from Intermediate 82 (0.20 g, 0.57 mmol), pyridine (1.9 mL), T3P® (50% in EtOAc, 1.10 mL, 1.7 mmol) and 2-tert-butoxycarbonyl-2-azaspiro[3.3]heptane-6-carboxylic acid (0.21 g, 0.86 mmol, CAS 1211526-53-2) at RT for 16 h. The crude product was purified by automated column chromatography (silica gel 12 g, eluting 0-50% EtOAc in isohexane). LCMS (Method L): 573.6 [M+H]⁺.

### Intermediate 88: tert-Butyl 6-(((1S)-1-(6-((1,1-dimethyl-2,3-dihydro-1H-inden-2-yl)amino)pyridin-3-yl)-2,2,2-trifluoroethyl)(methyl)carbamoyl)-2-azaspiro[3.3]heptane-2-carboxylate

The title compound (18 mg) was prepared in an analogous manner to Intermediate 3 from Intermediate 63 (0.10 g, 0.25 mmol), pyridine (1.0 mL), T3P® (50% in EtOAc, 1.49 mL, 0.75 mmol) and 2-tert-butoxycarbonyl-2-azaspiro[3.3]heptane-6-carboxylic acid (0.09 g, 0.37 mmol, CAS 1211526-53-2) at RT for 16 h. The crude product was purified by automated column chromatography (silica gel 12 g, eluting 0-40% EtOAc in hexane). LCMS (Method K): 573.6 [M+H]⁺.

### Intermediate 89: tert-Butyl (R)-3-(((S)-1-(5-(((S)-1,1-dimethyl-2,3-dihydro-1H-inden-2-yl)amino)pyridin-2-yl)-2,2,2-trifluoroethyl)(methyl)carbamoyl)pyrrolidine-1-carboxylate

The title compound (0.21 g) was prepared in an analogous manner to Intermediate 3 from Intermediate 82 (0.15 g, 0.43 mmol), pyridine (2.1 mL), T3P® (50% in EtOAc, 0.80 mL, 1.3 mmol) and (3R)-1-tert-butoxycarbonylpyrrolidine-3-carboxylic acid (0.14 g, 0.64 mmol, CAS 72925-16-7) at RT for 16 h. The crude product was purified by automated column chromatography (silica gel 12 g, eluting 0-20% EtOAc in hexane). LCMS (Method K): 547.1 [M+H]⁺.

### Intermediate 90: (S)—N-(1-(5-bromopyridin-2-yl)-2,2,2-trifluoroethyl)-N-methyltetrahydro-2H-thiopyran-4-carboxamide 1,1-dioxide

To a stirred solution of tetrahydro-2H-thiopyran-4-carboxylic acid 1,1-dioxide (1.54 g, 8.64 mmol, CAS 64096-87-3) in DCM (5 mL) at 0° C. was added oxalyl chloride (0.15 mL, 1.73 mmol) and DMF (0.1 mL) then the mixture was stirred at RT for 2 h. A solution of Intermediate 58 (0.50 g, 1.73 mmol) and triethylamine (0.24 mL, 1.72 mmol) in DCM (5 mL) premixed for 30 min at RT was cooled at 0° C. then the acid chloride mixture was added then the mixture stirred at RT for 2 h. The mixture was diluted with water and extracted with DCM. The organic layer washed with brine, dried over Na₂SO₄, filtered and concentrated under reduced pressure. The crude product was purified by flash chromatography (silica gel, eluting 0-45% EtOAc in petroleum ether) to provide the title compound (0.45 g). LCMS (Method A): 431.2 [M+H]⁺.

### Intermediate 91: tert-Butyl (3-(((S)-1-(5-(((S)-1,1-dimethyl-2,3-dihydro-1H-inden-2-yl)amino)pyridin-2-yl)-2,2,2-trifluoroethyl)(methyl)carbamoyl)bicyclo[1.1.1]pentan-1-yl)carbamate

The title compound (0.12 g) was prepared in an analogous manner to Intermediate 3 from Intermediate 82 (0.10 g, 0.29 mmol), pyridine (1.4 mL), T3P® (50% in EtOAc, 0.53 mL, 0.86 mmol) and 3-(tert-butoxycarbonylamino)bicyclo[1.1.1]pentane-1-carboxylic acid (68 mg, 0.30 mmol, CAS 303752-38-7) at RT for 16 h. The crude product was purified by automated column chromatography (silica gel 40 g, eluting 0-50% EtOAc in hexane). LCMS (Method J): 559.4 [M+H]⁺.

### Intermediate 92: 3-Amino-N—((S)-1-(5-(((S)-1,1-dimethyl-2,3-dihydro-1H-inden-2-yl)amino)pyridin-2-yl)-2,2,2-trifluoroethyl)-N-methylbicyclo[1.1.1]pentane-1-carboxamide Dihydrochloride

To a stirred solution of Intermediate 91 (0.11 g, 0.19 mmol) in 1,4-dioxane (1.8 mL) was added 4M HCl in 1,4-dioxane (0.29 μL) and stirred at RT for 16 h. Additional 4M HCl in 1,4-dioxane (0.29 μL) was added and stirred at RT for 16 h. The mixture was concentrated under reduced pressure and triturated in diethyl ether to provide the title compound (0.11 g). ¹H NMR (300 MHz; DMSO-d₆-D₂O shake) δ: 8.14 (s, 1H), 7.25-7.10 (m, 6H), 6.30 (dd, 1H), 4.03 (dd, 1H), 3.22-3.12 (m, 1H), 3.01 (s, 3H), 2.80-2.68 (m, 1H), 2.42 (d, 1H), 2.35 (s, 5H), 1.31 (s, 3H), 1.08 (s, 3H).

### Intermediate 93: tert-Butyl (R)-2-(((S)-1-(5-(((S)-1,1-dimethyl-2,3-dihydro-1H-inden-2-yl)amino)pyridin-2-yl)-2,2,2-trifluoroethyl)(methyl)carbamoyl)morpholine-4-carboxylate

The title compound (0.16 g) was prepared in an analogous manner to Intermediate 3 from Intermediate 82 (0.15 g, 0.41 mmol), pyridine (1.4 mL), T3P® (50% in EtOAc, 0.81 mL, 1.27 mmol) and (2R)-4-tert-butoxycarbonylmorpholine-2-carboxylic acid (99 mg, 0.43 mmol, CAS 884512-77-0) at RT for 16 h. The crude product was purified by automated column chromatography (silica gel 25 g, eluting 5-50% EtOAc in hexane). ¹H NMR (300 MHz; DMSO-d₆) δ: 8.16 (s, 1H), 7.33-7.09 (m, 6H), 6.27 (dd, 2H), 4.35-4.22 (m, 1H), 4.09-4.03 (m, 2H), 3.93-3.67 (m, 3H), 3.53 (t, 1H), 3.19 (dd, 1H), 3.00 (s, 3H), 2.80-2.68 (m, 2H), 1.42 (s, 9H), 1.33 (s, 3H), 1.10 (s, 3H).

### Intermediate 94: (R)—N—((S)-1-(5-(((S)-1,1-dimethyl-2,3-dihydro-1H-inden-2-yl)amino)pyridin-2-yl)-2,2,2-trifluoroethyl)-N-methylmorpholine-2-carboxamide Dihydrochloride

To a stirred solution of Intermediate 93 (0.15 g, 0.26 mmol) in 1,4-dioxane (2.6 mL) was added 4M HCl in 1,4-dioxane (0.39 mL) and stirred at RT for 16 h. The mixture was concentrated under reduced pressure and triturated in diethyl ether to provide the title compound (70 mg). LCMS (Method M): 463.3 [M+H]⁺.

### Intermediate 95: tert-Butyl (S)-2-(((S)-1-(5-(((S)-1,1-dimethyl-2,3-dihydro-1H-inden-2-yl)amino)pyridin-2-yl)-2,2,2-trifluoroethyl)(methyl)carbamoyl)morpholine-4-carboxylate

The title compound (0.26 g) was prepared in an analogous manner to Intermediate 3 from Intermediate 82 (0.20 g, 0.57 mmol), pyridine (1.9 mL), T3P® (50% in EtOAc, 1.05 mL, 1.71 mmol) and (2S)-4-tert-butoxycarbonylmorpholine-2-carboxylic acid (0.14 g, 0.60 mmol, CAS 868689-63-8) at RT for 16 h. The crude product was purified by automated column chromatography (silica gel 40 g, eluting 5-50% EtOAc in hexane). LCMS (Method J): 563.4 [M+H]⁺.

### Intermediate 96: (S)—N—((S)-1-(5-(((S)-1,1-dimethyl-2,3-dihydro-1H-inden-2-yl)amino)pyridin-2-yl)-2,2,2-trifluoroethyl)-N-methylmorpholine-2-carboxamide Dihydrochloride

To a stirred solution of Intermediate 95 (0.15 g, 0.26 mmol) in 1,4-dioxane (1.5 mL) was added 4M HCl in 1,4-dioxane (1.38 mL) and stirred at RT for 16 h. The mixture was concentrated under reduced pressure and triturated in diethyl ether to provide the title compound (0.17 g). LCMS (Method M): 463.3 [M+H]⁺.

### Intermediate 97: tert-Butyl ((1,3-cis)-3-(((S)-1-(5-(((S)-1,1-dimethyl-2,3-dihydro-1H-inden-2-yl)amino)pyridin-2-yl)-2,2,2-trifluoroethyl)(methyl)carbamoyl)cyclobutyl)carbamate

The title compound (0.16 g) was prepared in an analogous manner to Intermediate 3 from Intermediate 82 (0.10 g, 0.57 mmol), pyridine (4.3 mL), T3P® (50% in MeTHF, 0.53 mL, 0.87 mmol) and cis-3-(tert-butoxycarbonylamino)cyclobutanecarboxylic acid (68 mg, 0.31 mmol, CAS 1008773-79-2) at RT for 16 h. LCMS (Method K): 547.4 [M+H]⁺.

### Intermediate 98: (1,3-cis)-3-Amino-N—((S)-1-(5-(((S)-1,1-dimethyl-2,3-dihydro-1H-inden-2-yl)amino)pyridin-2-yl)-2,2,2-trifluoroethyl)-N-methylcyclobutane-1-carboxamide TFA Salt

To a stirred mixture of Intermediate 97 (0.17 g, 0.30 mmol) in DCM (1.5 mL) at 0° C. was added TFA (0.23 mL) and stirred at RT for 16 h. The mixture was concentrated under reduced pressure to provide the title compound (0.14 g). ¹H NMR (300 MHz; DMSO-d₆) δ: 8.14 (d, 1H), 7.27-7.00 (m, 6H), 6.35 (q, 1H), 6.21 (d, 1H), 3.26-3.14 (m, 2H), 2.86 (s, 3H), 2.81-2.63 (m, 2H), 2.44-2.22 (m, 1H), 1.91-1.64 (m, 3H), 1.32 (s, 3H), 1.10 (s, 3H)—NHs not observed.

### Intermediate 99: (2S,4R)-1-Acetyl-4-((tert-butyldimethylsilyl)oxv)pyrrolidine-2-carboxylic Acid

To a stirred solution of (2S,4R)-1-acetyl-4-hydroxypyrrolidine-2-carboxylic acid (0.20 g, 1.15 mmol, CAS 33996-33-7) in MeCN (1.4 mL) was added DBU (0.37 mL, 2.48 mmol) and TBDMS-CI (0.37 g, 2.48 mmol) and the mixture was stirred at RT for 3 h. The mixture was diluted with water and extracted with EtOAc. The organic layer was dried over Na₂SO₄, filtered and concentrated under reduced pressure. The crude product was triturated in diethyl ether to provide the title compound (0.15 g). ¹H NMR (300 MHz; DMSO-d₆) δ: 12.56 (br s, 1H), 4.56-4.45 (m, 1H), 4.20 (t, 1H), 3.70 (dd, 1H), 2.22-1.98 (m, 2H), 1.95 (s, 3H), 1.85 (s, 1H), 0.85 (d, 9H), 0.12-0.00 (m, 6H).

### Intermediate 100: (2S,4R)-1-Acetyl-4-((tert-butyldimethylsilyl)oxy)-N—((S)-1-(5-(((S)-1,1-dimethyl-2,3-dihydro-1H-inden-2-yl)amino)pyridin-2-yl)-2,2,2-trifluoroethyl)-N-methylpyrrolidine-2-carboxamide

The title compound (0.10 g) was prepared in an analogous manner to Intermediate 3 from Intermediate 82 (0.10 g, 0.29 mmol), pyridine (2.1 mL), T3P® (50% in MeTHF, 0.53 mL, 0.86 mmol) and Intermediate 99 (91 mg, 0.32 mmol) at RT for 16 h. Purified by prep-TLC (eluting DCM 4% MeOH). LCMS (Method K): 619.3 [M+H]⁺.

### Intermediate 101: Methyl 3-(((S)-1-(5-(((S)-1,1-dimethyl-2,3-dihydro-1H-inden-2-yl)amino)pyridin-2-yl)-2,2,2-trifluoroethyl)(methyl)carbamoyl)bicyclo[1.1.1]pentane-1-carboxylate

The title compound (0.16 g) was prepared in an analogous manner to Intermediate 3 from Intermediate 82 (0.10 g, 0.29 mmol), pyridine (4.3 mL), T3P® (50% in MeTHF, 0.53 mL, 0.87 mmol) and 3-methoxycarbonylbicyclo[1.1.1]pentane-1-carboxylic acid (54 mg, 0.32 mmol, CAS 83249-10-9) at RT for 16 h. LCMS (Method K): 4.01 min, 502.3 [M+H]⁺.

### Intermediate 102: tert-Butyl ((1,3-trans)-3-(((S)-1-(5-(((S)-1,1-dimethyl-2,3-dihydro-1H-inden-2-yl)amino)pyridin-2-yl)-2,2,2-trifluoroethyl)(methyl)carbamoyl)cyclobutyl)carbamate

The title compound (0.15 g) was prepared in an analogous manner to Intermediate 3 from Intermediate 82 (0.10 g, 0.29 mmol), pyridine (1.9 mL), T3P® (50% in MeTHF, 0.53 mL, 0.87 mmol) and trans-3-((tert-butoxycarbonyl)amino)cyclobutane carboxylic acid (54 mg, 0.32 mmol, CAS 939400-34-7) at RT for 16 h. LCMS (Method K): 547.3 [M+H]⁺.

### Intermediate 103: (1,3-trans)-3-Amino-N—((S)-1-(5-(((S)-1,1-dimethyl-2,3-dihydro-1H-inden-2-yl)amino)pyridin-2-yl)-2,2,2-trifluoroethyl)-N-methylcyclobutane-1-carboxamide

To a stirred solution of Intermediate 102 (0.15 g, 0.28 mmol) in 1,4-dioxane (1.2 mL), was added 2M HCl in diethyl ether (0.49 mL) at 0° C. and the mixture was stirred at RT for 16 h. The mixture was poured into 1M aqueous NaOH and extracted with DCM. The combined organics were dried over Na₂SO₄, filtered and concentrated under reduced pressure to provide the title compound (0.13 g). LCMS (Method K): 447.0 [M+H]⁺.

### Intermediate 104: tert-Butyl ((1R,3S)-3-(((S)-1-(5-(((S)-1,1-dimethyl-2,3-dihydro-1H-inden-2-yl)amino)pyridin-2-yl)-2,2,2-trifluoroethyl)(methyl)carbamoyl)cyclopentyl)carbamate

The title compound (0.15 g) was prepared in an analogous manner to Intermediate 3 from Intermediate 82 (0.10 g, 0.29 mmol), pyridine (2.1 mL), T3P® (50% in MeTHF, 0.53 mL, 0.87 mmol) and (1S,3R)-3-(tert-butoxycarbonylamino)cyclopentane carboxylic acid (69 mg, 0.30 mmol, CAS 261165-05-3) at RT for 16 h. LCMS (Method K): 561.3 [M+H]⁺.

### Intermediate 105: (1S,3R)-3-Amino-N—((S)-1-(5-(((S)-1,1-dimethyl-2,3-dihydro-1H-inden-2-yl)amino)pyridin-2-yl)-2,2,2-trifluoroethyl)-N-methylcyclopentane-1-carboxamide

To a stirred solution of Intermediate 104 (0.15 g, 0.27 mmol) in DCM (1.7 mL), was added 2M HCl in diethyl ether (0.75 mL) at 0° C. and the mixture was stirred at RT for 16 h. The mixture was poured into 1M aqueous NaOH and extracted with DCM. The combined organics were washed with brine, dried over Na₂SO₄, filtered and concentrated under reduced pressure to provide the title compound (0.11 g). LCMS (Method K): 461.2 [M+H]⁺.

### Intermediate 106: (S)-1-(5-Bromopyridin-2-yl)-N-ethyl-2,2,2-trifluoroethan-1-amine

The title compound (2 g) was prepared in an analogous manner to Intermediate 24 from 1-(5-bromopyridin-2-yl)-2,2,2-trifluoroethyl trifluoromethanesulfonate (7.0 g, 18.0 mmol, CAS 1374038-21-7) and ethylamine in THF (2M, 90 mL) at 60° C. for 2 h. Purified by flash chromatography (silica gel, eluting 8-10% EtOAc in petroleum ether). Chiral purification was carried out by preparative SFC (Chiralpak®-IG, 30×250 mm×5 μm, temp 30° C., Back Pressure 100 bar, flow rate: 95 g/min, 95% CO₂ with 5% IPA modifier) as Peak 2. LCMS (Method A): 283.1 [M+H]⁺.

### Intermediate 107: (S)—N-(1-(5-Bromopyridin-2-yl)-2,2,2-trifluoroethyl)-N-ethyltetrahydro-2H-thiopyran-4-carboxamide 1,1-dioxide

The title compound (0.65 g) was prepared in an analogous manner to Intermediate 90 from Intermediate 106 (1.20 g, 4.15 mmol), pyridine (1.68 mL, 20.8 mmol) in THF (12 mL) and tetrahydro-2H-thiopyran-4-carboxylic acid 1,1-dioxide (2.22 g, 12.5 mmol, CAS 64096-87-3), oxalyl chloride (1.75 mL, 20.77 mmol) and DMF (0.03 mL) in DCM (12 mL) at 0° C. then at 40° C. for 16 h. Purified by flash chromatography (silica gel, eluting 40-50% EtOAc in petroleum ether) to provide the title compound (0.45 g). LCMS (Method A): 443.2 [M+H]⁺.

### Intermediate 108: (E)-2-(Hydroxyimino)-4-(trifluoromethyl)-2,3-dihydro-1H-inden-1-one

To a stirred solution of 4-(trifluoromethyl)indan-1-one (1.0 g, 5.00 mmol, CAS 68755-42-0) and acetyl chloride (0.14 mL, 2.00 mmol) in MeOH (0.5 mL), heptane (6 mL) and DCM (7 mL) at 0° C. was added isoamyl nitrite (1.01 mL, 7.49 mmol) dropwise and and the mixture was stirred at 0° C. for 2 h. Heptane was added and the solid was filtered and dried under reduced pressure to provide the title compound (0.50 g). LCMS (Method A): 230.1 [M+H]⁺.

### Intermediate 109: 4-(Trifluoromethyl)-2,3-dihydro-1H-inden-2-amine Hydrochloride

To a stirred solution of Intermediate 108 (0.50 g, 2.12 mmol) in AcOH (15 mL), was added sulfuric acid (2 mL, 37.5 mmol) slowly and the mixture was stirred at RT for 15 min. Palladium on carbon (10%, 0.50 g) was added and the mixture stirred under a hydrogen atmosphere for 16 h. The mixture was filtered through Celite, washed with EtOAc and the filtrate was basified carefully with Na₂CO₃ and extracted with EtOAc. The organic layer was acidified with 4M HCl in 1,4-dioxane and concentrated under reduced pressure to provide the title compound (0.39 g). LCMS (Method A): 202.2 [M+H]⁺.

### Intermediate 110: (E)-4-Ethyl-2-(hydroxyimino)-2,3-dihydro-1H-inden-1-one

To a stirred solution of 4-ethylindan-1-one (5.5 g, 29.5 mmol, CAS 79780-68-0) in MeOH (55 mL) at 40° C. was added isoamyl nitrite (3.86 mL, 32.5 mmol) and the mixture was stirred at 40° C. for 1 h. Hydrochloric acid (12 M, 5.5 mL) was added and the mixture was stirred at 60° C. for 16 h. The mixture was cooled and ice/water added and the solid was filtered and dried under reduced pressure. The crude product was purified by flash chromatography (neutral silica gel, eluting 5-10% MeOH in DCM) to provide the title compound (2.50 g). LCMS (Method A): 190.1 [M+H]⁺.

### Intermediate 111: 4-Ethyl-2,3-dihydro-1H-inden-2-amine Hydrochloride

The title compound (1.10 g) was prepared in an analogous manner to Intermediate 109 from Intermediate 110 (2.0 g, 9.51 mmol) in AcOH (45 mL), sulfuric acid (4.5 mL, 84.4 mmol), palladium on carbon (10%, 2.20 g) under a hydrogen atmosphere for 16 h at 450 psi, acidified with 4M HCl in 1,4-dioxane. LCMS (Method O): 162.0 [M+H]⁺.

### Intermediate 112: N-((1S)-1-(5-((5-chloro-2,3-dihydro-1H-inden-2-yl)amino)pyridin-2-yl)-2,2,2-trifluoroethyl)-N-methylpivalamide

The title compound (0.40 g) was prepared in an analogous manner to Intermediate 11 from Intermediate 59 (1.0 g, 2.63 mmol), 5-chloro-2,3-dihydro-1H-inden-2-amine hydrochloride (0.44 g, 2.63 mmol, CAS 73536-86-4), Cs₂CO₃ (2.57 g, 7.90 mmol), and RuPhosPdG3 (0.22 g, 0.26 mmol) in toluene (20 mL) at 110° C. for 16 h. Purified by flash chromatography (silica gel, eluting 20-25% EtOAc in petroleum ether). LCMS (Method C): 440.4 [M+H]⁺.

### Intermediate 113: N-(5-Chloro-2,3-dihydro-1H-inden-2-yl)-6-((S)-2,2,2-trifluoro-1-(methylamino)ethyl)pyridin-3-amine

The title compound (0.25 g) was prepared in an analogous manner to Intermediate 61 from Intermediate 112 (0.40 g, 0.45 mmol) and LiAlH₄ (2M in THF, 0.98 mL, 1.96 mmol) in THF (8.0 mL) at −0° C. for 30 min. The crude product was purified by flash chromatography (silica gel, eluting 25-30% EtOAc in petroleum ether). LCMS (Method A): 356.3 [M+H]⁺.

### Intermediate 115: N—((S)-1-(5-(((R)-1,1-Dimethyl-2,3-dihydro-1H-inden-2-yl)amino)pyridin-2-yl)-2,2,2-trifluoroethyl)-N-methylpivalamide

The title compound (1.29 g) was prepared in an analogous manner to Intermediate 11 from Intermediate 59 (1.5 g, 4.25 mmol), Intermediate 114 (0.90 g, 5.58 mmol), Cs₂CO₃ (4.15 g, 12.7 mmol), and RuPhosPdG3 (0.51 g, 0.14 mmol) in toluene (14.2 mL) at 110° C. for 20 h. Purified by automated column chromatography (silica gel 120 g, eluting 0-40% EtOAc in hexane). ¹H NMR (300 MHz; DMSO-d₆) δ: 8.16 (d, 1H), 7.26-7.13 (m, 5H), 7.12-7.02 (d, 1H), 6.45 (dd, 1H), 6.20 (d, 1H), 4.19-3.95 (m, 1H), 3.21 (dd, 1H), 3.03 (s, 3H), 2.75 (dd, 1H), 1.33 (s, 3H), 1.26 (s, 9H), 1.12 (s, 3H).

### Intermediate 116: N—((R)-1,1-Dimethyl-2,3-dihydro-1H-inden-2-yl)-6-((S)-2,2,2-trifluoro-1-(methylamino)ethyl)pyridin-3-amine

The title compound (0.88 g) was prepared in an analogous manner to Intermediate 61 from Intermediate 115 (1.29 g, 2.97 mmol) and LiAlH₄ (2M in THF, 3.00 mL, 6.00 mmol) in THF (30 mL) at −15° C. for 30 min. The crude product was purified by automated column chromatography (silica gel 80 g, eluting 0-50% EtOAc in hexane). ¹H NMR (300 MHz; DMSO-d₆) δ: 8.12 (d, 1H), 7.25-7.08 (m, 6H), 6.06 (d, 1H), 4.06 (dq, 2H), 3.20 (dd, 1H), 2.75 (dd, 1H), 2.67-2.59 (m, 1H), 2.24 (d, 3H), 1.32 (s, 3H), 1.10 (s, 3H).

### Intermediate 117: Methyl 3-(((S)-1-(5-(((R)-1,1-dimethyl-2,3-dihydro-1H-inden-2-yl)amino)pyridin-2-yl)-2,2,2-trifluoroethyl)(methyl)carbamoyl)bicyclo[1.1.1]pentane-1-carboxylate

The title compound (0.17 g) was prepared in an analogous manner to Intermediate 3 from Intermediate 116 (0.10 g, 0.29 mmol), pyridine (1.4 mL), T3P® (50% in MeTHF, 0.53 mL, 0.87 mmol) and 3-methoxycarbonylbicyclo[1.1.1]pentane-1-carboxylic acid (54 mg, 0.32 mmol, CAS 83249-10-9) at RT for 16 h. LCMS (Method K): 502.4 [M+H]⁺.

### Intermediate 118: tert-Butyl ((1S,3R)-3-(((S)-1-(5-(((R)-1,1-dimethyl-2,3-dihydro-1H-inden-2-yl)amino)pyridin-2-yl)-2,2,2-trifluoroethyl)(methyl)carbamoyl)cyclopentyl) carbamate

The title compound (0.19 g) was prepared in an analogous manner to Intermediate 3 from Intermediate 116 (0.10 g, 0.29 mmol), pyridine (1.4 mL), T3P® (50% in MeTHF, 0.55 mL, 0.87 mmol) and (1R,3S)-3-(tert-butoxycarbonylamino)cyclopentane carboxylic acid (72 mg, 0.32 mmol, CAS 161660-94-2) at RT for 16 h. LCMS (Method J): 561.3 [M+H]⁺.

### Intermediate 119: (1R,3S)-3-Amino-N—((S)-1-(5-(((R)-1,1-dimethyl-2,3-dihydro-1H-inden-2-yl)amino)pyridin-2-yl)-2,2,2-trifluoroethyl)-N-methylcyclopentane-1-carboxamide Dihydrochloride

To a stirred solution of Intermediate 118 (0.19 g, 0.27 mmol) in 1,4-dioxane (1.8 mL) was added 4M HCl in 1,4-dioxane (0.81 mL) and stirred at RT for 16 h. The mixture was concentrated under reduced pressure and triturated in n-pentane to provide the title compound (0.20 g). ¹H NMR (300 MHz; DMSO-d₆ 2 drops D₂O) δ: 8.19-8.02 (m, 2H), 7.29-7.08 (m, 6H), 6.40 (q, 1H), 4.04 (t, 1H), 3.51 (dd, 1H), 3.30-3.10 (m, 2H), 2.98 (s, 3H), 2.82-2.65 (m, 1H), 2.37-1.46 (m, 6H), 1.31 (s, 3H), 1.08 (s, 3H).

### Intermediate 120: tert-Butyl ((1,3-cis)-3-(((S)-1-(5-(((R)-1,1-dimethyl-2,3-dihydro-1H-inden-2-yl)amino)pyridin-2-yl)-2,2,2-trifluoroethyl)(methyl)carbamoyl)cyclobutyl) carbamate

The title compound (0.11 g) was prepared in an analogous manner to Intermediate 3 from Intermediate 116 (0.10 g, 0.29 mmol), pyridine (1.4 mL), T3P® (50% in MeTHF, 0.55 mL, 0.86 mmol) and cis-3-(tert-butoxycarbonylamino)cyclobutane carboxylic acid (68 mg, 0.31 mmol, CAS 1008773-79-2) at RT for 16 h. ¹H NMR (300 MHz; DMSO-d₆) δ: 8.58 (d, 1H), 8.14 (d, 1H), 7.79 (t, 1H), 7.39 (dd, 1H), 7.31-7.04 (m, 5H), 6.39 (d, 1H), 6.20 (d, 1H), 4.14-3.88 (m, 2H), 3.20 (dd, 1H), 2.92-2.63 (m, 4H), 2.40-2.07 (m, 4H), 1.38 (s, 9H), 1.32 (s, 3H), 1.10 (s, 3H).

### Intermediate 121: (1,3-cis)-3-Amino-N—((S)-1-(5-(((R)-1,1-dimethyl-2,3-dihydro-1H-inden-2-yl)amino)pyridin-2-yl)-2,2,2-trifluoroethyl)-N-methylcyclobutane-1-carboxamide Dihydrochloride

To a stirred solution of Intermediate 120 (0.10 g, 0.19 mmol) in diethyl ether (0.9 mL) was added 2M HCl in diethyl ether (0.93 mL) and stirred at RT for 16 h. The mixture was concentrated under reduced pressure to provide the title compound (90 mg). LCMS (Method K): 447.8 [M+H]⁺.

### Intermediate 122: tert-Butyl ((1,3-trans)-3-(((S)-1-(5-(((R)-1,1-dimethyl-2,3-dihydro-1H-inden-2-yl)amino)pyridin-2-yl)-2,2,2-trifluoroethyl)(methyl)carbamoyl)cyclobutyl) carbamate

The title compound (0.18 g) was prepared in an analogous manner to Intermediate 3 from Intermediate 116 (0.10 g, 0.29 mmol), pyridine (1.4 mL), T3P® (50% in MeTHF, 0.55 mL, 0.86 mmol) and trans-3-(tert-butoxycarbonylamino)cyclobutane carboxylic acid (68 mg, 0.31 mmol, CAS 939400-34-7) at RT for 16 h. LCMS (Method K): 547.8 [M+H]⁺.

### Intermediate 123: (1,3-trans)-3-Amino-N—((S)-1-(5-(((R)-1,1-dimethyl-2,3-dihydro-1H-inden-2-yl)amino)pyridin-2-yl)-2,2,2-trifluoroethyl)-N-methylcyclobutane-1-carboxamide Dihydrochloride

To a stirred solution of Intermediate 122 (0.16 g, 0.29 mmol) in diethyl ether (0.9 mL) was added 2M HCl in diethyl ether (1.43 mL) and stirred at RT for 16 h. The mixture was concentrated under reduced pressure to provide the title compound (90 mg). LCMS (Method K): 447.6 [M+H]⁺.

### Intermediate 124: N—((S)-1,1-Dimethyl-2,3-dihydro-1H-inden-2-yl)-5-((S)-2,2,2-trifluoro-1-(methylamino)ethyl)pyridin-2-amine

The title compound (0.34 g) was prepared in an analogous manner to Intermediate 26 from Intermediate 62 (0.90 g, 3.35 mmol), Intermediate 80 (0.64 g, 4.01 mmol), sodium t-butoxide (0.97 g, 10.0 mmol), Pd₂(dba)₃ (0.31 g, 0.33 mmol) and X-Phos (0.32 g, 0.70 mmol) in toluene (33 mL) at 110° C. for 16 h. Purified by automated column chromatography (silica gel, 40 g, eluting 0-40% EtOAc in hexane). LCMS (Method K): 350.6 [M+H]⁺.

### Intermediate 125: (2S,4R)-1-Acetyl-4-((tert-butyldimethylsilyl)oxy)-N—((S)-1-(5-(((R)-1,1-dimethyl-2,3-dihydro-1H-inden-2-yl)amino)pyridin-2-yl)-2,2,2-trifluoroethyl)-N-methylpyrrolidine-2-carboxamide

The title compound (0.11 g) was prepared in an analogous manner to Intermediate 3 from Intermediate 116 (75 mg, 0.21 mmol), pyridine (1.4 mL), T3P® (50% in MeTHF, 0.39 mL, 0.64 mmol) and Intermediate 99 (68 mg, 0.24 mmol) at RT for 16 h. LCMS (Method K): 619.3 [M+H]⁺.

### Intermediate 126: (1,3-cis)-3-Amino-N—((S)-1-(5-(((S)-1,1-dimethyl-2,3-dihydro-1H-inden-2-yl)amino)pyridin-2-yl)-2,2,2-trifluoroethyl)-N-methylcyclobutane-1-carboxamide Dihydrochloride

To a stirred solution of Intermediate 97 (0.23 g, 0.42 mmol) in 1,4-dioxane (2.9 mL) was added 4M HCl in 1,4-dioxane (1.26 mL) and stirred at RT for 16 h. The mixture was concentrated under reduced pressure and triturated with pentane to provide the title compound (0.22 g). LCMS (Method K): 447.1 [M+H]⁺.

### Intermediate 127: tert-Butyl 4-(((S)-1-(5-(((S)-1,1-dimethyl-2,3-dihydro-1H-inden-2-yl)amino)pyridin-2-yl)-2,2,2-trifluoroethyl)(methyl)carbamoyl)piperidine-1-carboxylate

The title compound (0.47 g) was prepared in an analogous manner to Intermediate 3 from Intermediate 82 (0.30 g, 0.86 mmol), pyridine (1.7 mL), T3P® (0.77 mL, 2.58 mmol) and 1-(tert-butoxycarbonyl)piperidine-4-carboxylic acid (0.22 g, 0.94 mmol, CAS 84358-13-4) at RT for 16 h. Crude product was purified by automated column chromatography (silica gel 40 g, eluting 0-100% EtOAc in hexane). ¹H NMR (400 MHz; DMSO-d₆) δ: 8.20-8.09 (m, 1H), 7.25-7.04 (m, 6H), 6.38 (q, 1H), 6.22 (d, 1H), 4.16-3.92 (d, 4H), 3.20 (dd, 1H), 3.02 (s, 3H), 2.93 (t, 1H), 2.90-2.67 (m, 3H), 1.84-1.50 (m, 3H), 1.40 (d, 9H), 1.33 (s, 3H), 1.11 (s, 3H).

### Intermediate 128: N—((S)-1-(5-(((S)-1,1-dimethyl-2,3-dihydro-1H-inden-2-yl)amino)pyridin-2-yl)-2,2,2-trifluoroethyl)-N-methylpiperidine-4-carboxamide Hydrochloride

To a stirred solution of Intermediate 127 (0.47 g, 0.59 mmol) in 1,4-dioxane (1.2 mL) was added 4M HCl in 1,4-dioxane (0.74 mL) and stirred at RT for 2 h. The mixture was concentrated under reduced pressure to provide the title compound (0.24 g). LCMS (Method K): 461.2 [M+H]⁺.

### Intermediate 129: tert-Butyl 3-(((1S)-1-(5-((1,1-dimethyl-2,3-dihydro-1H-inden-2-yl)amino)pyridin-2-yl)-2,2,2-trifluoroethyl)(methyl)carbamoyl)azetidine-1-carboxylate

The title compound (0.35 g) was prepared in an analogous manner to Intermediate 3 from Intermediate 61 (0.25 g, 0.67 mmol), pyridine (0.3 mL), T3P® (50% in MeTHF, 2.12 mL, 6.65 mmol) and 1-(tert-butoxycarbonyl)azetidine-3-carboxylic acid (0.21 g, 0.99 mmol, CAS 142253-55-2) at RT for 16 h. Purified by flash chromatography (silica gel, eluting 0-60% EtOAc in petroleum ether). LCMS (Method A): 533.6 [M+H]⁺.

### Intermediate 130: N-((1S)-1-(5-((1,1-Dimethyl-2,3-dihydro-1H-inden-2-yl)amino)pyridin-2-yl)-2,2,2-trifluoroethyl)-N-methylazetidine-3-carboxamide TFA Salt

A solution of Intermediate 129 (0.35 g, 0.54 mmol) in TFA (0.23 mL) and DCM (5 mL) was stirred at RT for 6 h. The mixture was and concentrated under reduced pressure to provide the title compound (0.32 g). LCMS (Method A): 433.5 [M+H]⁺.

### Intermediate 131: Methyl (1,3-cis)-3-(methylcarbamoyl)cyclobutane-1-carboxylate

To a stirred solution of cis-3-(methoxycarbonyl)cyclobutanecarboxylic acid (50 mg, 0.32 mmol, CAS 142733-61-7) in DCM (1.6 mL) at 0° C. was added oxalyl chloride (32 μL, 0.38 mmol) then the mixture was stirred at RT for 2 h. Methylamine (1M in THF, 1.58 mL, 1.58 mmol) was added then the mixture stirred at RT for 16 h. The mixture was concentrated under reduced pressure to provide the title compound (75 mg). ¹H NMR (300 MHz; DMSO-d₆) δ: 7.71 (br s, 1H), 3.58 (s, 3H), 3.11-2.99 (m, 1H), 2.97-2.73 (m, 1H), 2.54 (d, 3H), 2.25-2.05 (m, 4H).

### Intermediate 132: (1,3-cis)-3-(Methylcarbamoyl)cyclobutane-1-carboxylic Acid

To a stirred solution of Intermediate 131 (75 mg, 0.44 mmol) in THF (3 mL) and MeOH (1.4 mL) was added lithium hydroxide (21 mg, 0.88 mmol) and the mixture was stirred at RT for 3 h. The mixture was concentrated under reduced pressure and dissolved in MeOH and treated with Amberlite® (IR 120H form) and filtered to provide the title compound (69 mg). ¹H NMR (300 MHz; DMSO-d₆) δ: 7.67 (s, 1H), 2.99-2.72 (m, 2H), 2.54 (d, 3H), 2.27-2.10 (m, 4H)—OH not observed.

### Intermediate 133: (2S,4R)-1-(tert-butoxycarbonyl)-4-((tert-butyldimethylsilyl)oxy)pyrrolidine-2-carboxylic Acid

The title compound (0.62 g) was prepared in an analogous manner to Intermediate 99 using (2S,4R)-1-(tert-butoxycarbonyl)-4-hydroxypyrrolidine-2-carboxylic acid (0.50 g, 2.16 mmol, CAS 13726-69-7), DBU (0.68 mL, 4.54 mmol) and TBDMS-CI (0.36 g, 2.38 mmol) in MeCN (4.3 mL) at RT for 18 h. ¹H NMR (300 MHz; DMSO-d₆) δ: 12.67 (br s, 1H), 4.42 (t, 1H), 4.12 (q, 1H), 3.43 (ddd, 1H), 3.20 (dt, 1H), 2.13-1.95 (m, 2H), 1.37 (d, 9H), 0.85 (s, 9H), 0.06 (d, 6H).

### Intermediate 134: tert-Butyl (2S,4R)-4-((tert-butyldimethylsilyl)oxy)-2-(((S)-1-(5-(((S)-1,1-dimethyl-2,3-dihydro-1H-inden-2-yl)amino)pyridin-2-yl)-2,2,2-trifluoroethyl)(methyl)carbamoyl)pyrrolidine-1-carboxylate

The title compound (0.35 g) was prepared in an analogous manner to Intermediate 3 from Intermediate 82 (0.20 g, 0.57 mmol), pyridine (0.3 mL), T3P® (50% in MeTHF, 1.09 mL, 1.72 mmol) and Intermediate 133 (0.22 g, 0.63 mmol) at RT for 16 h. Purified by automated column chromatography (silica gel 40 g, eluting 0-40% EtOAc in hexanes). LCMS (Method J): 677.2 [M+H]⁺.

### Intermediate 135: (2S,4R)—N—((S)-1-(5-(((S)-1,1-dimethyl-2,3-dihydro-1H-inden-2-yl)amino)pyridin-2-yl)-2,2,2-trifluoroethyl)-4-hydroxy-N-methylpyrrolidine-2-carboxamide Dihydrochloride

To a stirred solution of Intermediate 134 (0.21 g, 0.31 mmol) in 1,4-dioxane (1.6 mL) was added 4M HCl in 1,4-dioxane (0.78 mL) and stirred at RT for 16 h. The mixture was concentrated under reduced pressure to provide the title compound (0.17 g). LCMS (Method J): 463.5 [M+H]⁺.

### Intermediate 136: Methyl (1,3-trans)-3-(methylcarbamoyl)cyclobutane-1-carboxylate

To a stirred solution of methyl trans-3-(methylcarbamoyl)cyclobutane-1-carboxylate (100 mg, 0.63 mmol, CAS 1401103-71-6) in DCM (3.2 mL) at 0° C. was added oxalyl chloride (64 μL, 0.76 mmol) then the mixture was stirred at RT for 2 h. Methylamine (1M in THF, 3.16 mL, 3.16 mmol) was added then the mixture stirred at RT for 16 h. The mixture was concentrated under reduced pressure to provide the title compound (0.20 g). ¹H NMR (300 MHz; DMSO-d₆) δ: 7.73 (br s, 1H), 3.61 (s, 3H), 3.15-2.93 (m, 2H), 2.56 (d, 3H), 2.33-2.16 (m, 4H).

### Intermediate 137: (1,3-trans)-3-(Methylcarbamoyl)cyclobutane-1-carboxylic Acid

To a stirred solution of Intermediate 136 (0.20 g, 1.17 mmol) in THF (2.2 mL) and MeOH (1.1 mL) was added lithium hydroxide (56 mg, 2.34 mmol) and the mixture was stirred at RT for 3 h. The mixture was concentrated under reduced pressure and dissolved in MeOH and treated with Amberlite® (IR 120H form) and filtered to provide the title compound (0.19 g). ¹H NMR (300 MHz; DMSO-d₆) δ: 7.65 (br d, 1H), 2.99-2.80 (m, 2H), 2.55 (dd, 3H), 2.30-2.14 (m, 4H)—OH not observed.

### Intermediate 138: N-((1S)-1-(5-((1,1-dimethyl-2,3-dihydro-1H-inden-2-yl)amino)pyridin-2-yl)-2,2,2-trifluoroethyl)-N-methyl-1-azaspiro[3.3]heptane-6-carboxamide TFA Salt

To a stirred mixture of Intermediate 79a (0.48 g, 0.72 mmol) in DCM (9.6 mL) at 0° C. was added TFA (2.5 mL) and stirred at RT for 6 h. The mixture was concentrated under reduced pressure and triturated in pentane to provide the title compound (0.60 g). LCMS (Method A): 473.2 [M+H]⁺.

### Intermediate 139: 1-(2-((tert-Butyldimethylsilyl)oxy)ethyl)-N-((1S)-1-(5-((1,1-dimethyl-2,3-dihydro-1H-inden-2-yl)amino)pyridin-2-yl)-2,2,2-trifluoroethyl)-N-methyl-1-azaspiro[3.3]heptane-6-carboxamide

To a stirred solution of Intermediate 138 (0.60 g, 0.93 mmol) in DMF (6 mL) was added DIPEA (0.49 mL, 2.79 mmol) followed by a solution of (2-bromoethoxy)-tert-butyl-dimethylsilane (0.89 g, 3.72 mmol, CAS 86864-60-0) in DMF (6 mL) over 5 min. The mixture was stirred at 70° C. for 16 h. The mixture was diluted in water and extracted with EtOAc. The organics were washed with water, brine, dried over Na₂SO₄, filtered and concentrated under reduced pressure. The crude product was purified by flash chromatography (silica gel, eluting 100% EtOAc) to provide the title compound (0.20 g). LCMS (Method A): 631.7 [M+H]⁺.

### Intermediate 140: 4-Bromo-2,3-dihydro-1H-inden-2-amine Hydrochloride

The title compound (1.40 g) was prepared in an analogous manner to Intermediate 6 using 4-bromoindan-2-one (10 g, 47.4 mmol, CAS 846032-36-8) ammonium acetate (54.8 g, 0.71 mol) and sodium cyanoborohydride (3.57 g, 56.9 mmol) in ethanol (0.2 L) at 100° C. for 3 h. Quenched with 4M HCl in 1,4-dioxane. LCMS (Method A): 1.05 min, 214.2 [M+H]⁺.

### Intermediate 141: tert-Butyl (4-bromo-2,3-dihydro-1H-inden-2-yl)carbamate

To a stirred solution of Intermediate 140 (1.40 g, 4.84 mmol) in DCM (30 mL) at 0° C. was added triethylamine (2.03 mL, 14.5 mmol) and di-tert-butyl dicarbonate (1.27 g, 5.82 mmol) and stirred for 3 h at RT. The mixture was poured into water, then extracted with DCM. The organic layer washed with water, brine, dried over Na₂SO₄, filtered and concentrated under reduced pressure. The crude product was purified by flash chromatography (silica gel, eluting 3-10% EtOAc in isohexane) to provide the title compound (1.80 g). LCMS (Method A): 256.1 [M−tBu+H]⁺.

### Intermediate 142: tert-Butyl (4-cyclopropyl-2,3-dihydro-1H-inden-2-yl)carbamate

To a stirred solution of Intermediate 141 (0.51 g, 1.60 mmol) in toluene (5 mL) and water (1 mL) was added potassium cyclopropyltrifluoroborate (0.36 g, 2.40 mmol) and the mixture was degassed with argon. Palladium acetate (36 mg, 0.16 mmol) and tricyclohexyl phosphine (90 mg, 0.32 mmol) were added and degassed with argon. K₃PO₄ (1.02 g, 4.81 mmol) was added and the mixture stirred at 100° C. for 16 h. The mixture was poured into water, then extracted with EtOAc. The organic layer washed with water, brine, dried over Na₂SO₄, filtered and concentrated under reduced pressure. The crude product was purified by automated column chromatography (silica gel, eluting 0-20% EtOAc in petroleum ether) to provide the title compound (0.36 g). LCMS (Method P): 218.2 [M−tBu+H]⁺.

### Intermediate 143: 4-Cyclopropyl-2,3-dihydro-1H-inden-2-amine Hydrochloride

To a stirred solution of Intermediate 142 (0.35 g, 1.08 mmol) in DCM (3 mL) was added 4M HCl in 1,4-dioxane (5 mL) and stirred at RT for 3 h. The mixture was concentrated under reduced pressure to provide the title compound (0.26 g). LCMS (Method C): 174.5 [M+H]⁺.

### Intermediate 144: N-Methyl-N-((1S)-2,2,2-trifluoro-1-(5-((5-(trifluoromethyl)-2,3-dihydro-1H-inden-2-yl)amino)pyridin-2-yl)ethyl)pivalamide

The title compound (1.80 g) was prepared in an analogous manner to Intermediate 11 from Intermediate 59 (1.0 g, 2.63 mmol), 5-(trifluoromethyl)indan-2-amine hydrochloride (0.73 g, 2.90 mmol, CAS 2851992-32-8), Cs₂CO₃ (2.57 g, 7.90 mmol), and RuPhosPdG3 (0.22 g, 0.26 mmol) in toluene (20 mL) at 110° C. for 16 h. Purified by flash chromatography (silica gel, eluting 15-25% EtOAc in petroleum ether). LCMS (Method A): 474.5 [M+H]⁺.

### Intermediate 145: 6-((S)-2,2,2-Trifluoro-1-(methylamino)ethyl)-N-(5-(trifluoromethyl)-2,3-dihydro-1H-inden-2-yl)pyridin-3-amine

The title compound (1.50 g) was prepared in an analogous manner to Intermediate 61 from Intermediate 144 (1.80 g, 3.19 mmol) and LiAlH₄ (2M in THF, 5.0 mL, 10.0 mmol) in THF (18 mL) at 0° C. for 30 min. The crude product was purified by flash chromatography (silica gel, eluting 25-30% EtOAc in petroleum ether). LCMS (Method A): 390.4 [M+H]⁺.

### Intermediate 146: 2-Amino-2,3-dihydro-1H-indene-4-carbonitrile Hydrochloride

To a stirred solution of tert-butyl (4-cyano-2,3-dihydro-1H-inden-2-yl)carbamate (0.11 g, 0.40 mmol, CAS 2116717-24-7) in 1,4-dioxane (1.1 mL) was added 4M HCl in 1,4-dioxane (2 mL) and stirred at RT for 2 h. The mixture was concentrated under reduced pressure to provide the title compound (80 mg). LCMS (Method A): 159.2 [M+H]⁺.

### Intermediate 147: (S)—N-(1-(5-((5,6-Dimethyl-2,3-dihydro-1H-inden-2-yl)amino)pyridin-2-yl)-2,2,2-trifluoroethyl)-N-methylpivalamide

The title compound (0.12 g) was prepared in an analogous manner to Intermediate 11 from Intermediate 59 (0.21 g, 0.60 mmol), 5,6-dimethylindan-2-amine (0.10 g, 0.62 mmol, CAS 162752-07-0), Cs₂CO₃ (0.58 g, 1.78 mmol), and RuPhosPdG3 (75 mg, 1.78 mmol) in toluene (3.1 mL) at 110° C. for 14 h. Purified by flash chromatography (silica gel, eluting 15-25% EtOAc in petroleum ether). ¹H NMR (300 MHz; DMSO-d₆) δ: 8.01 (d, 1H), 7.16-6.93 (m, 4H), 6.50-6.32 (m, 2H), 4.22 (q, 1H), 3.24 (dd, 2H), 3.02 (s, 3H), 2.72 (dd, 2H), 2.18 (s, 6H), 1.25 (s, 9H).

### Intermediate 148: (S)—N-(5,6-Dimethyl-2,3-dihydro-1H-inden-2-yl)-6-(2,2,2-trifluoro-1-(methylamino)ethyl)pyridin-3-amine

The title compound (47 mg) was prepared in an analogous manner to Intermediate 61 from Intermediate 147 (0.12 g, 0.27 mmol) and LiAlH₄ (2M in THF, 0.27 mL, 0.53 mmol) in THF (1.3 mL) at −15° C. for 30 min. The crude product was purified by automated column chromatography (silica gel 12 g, eluting 0-50% EtOAc in hexane). ¹H NMR (300 MHz; DMSO-d₆) δ: 7.98 (d, 1H), 7.23 (d, 1H), 7.03-6.96 (m, 3H), 6.27 (d, 1H), 4.29-4.08 (m, 2H), 3.24 (dd, 2H), 2.72 (dd, 2H), 2.63 (br s, 1H), 2.23 (s, 3H), 2.18 (s, 6H).

### Intermediate 149: N-((1S)-1-(5-((4-chloro-2,3-dihydro-1H-inden-2-yl)amino)pyridin-2-yl)-2,2,2-trifluoroethyl)-N-methylpivalamide

The title compound (0.80 g) was prepared in an analogous manner to Intermediate 11 from Intermediate 59 (0.70 g, 1.94 mmol), 4-chloroindan-2-amine hydrochloride (0.45 g, 2.14 mmol, CAS 2408959-26-0), Cs₂CO₃ (1.90 g, 5.83 mmol), and RuPhosPdG3 (0.32 g, 0.39 mmol) in toluene (7 mL) at 110° C. for 16 h. Purified by automated column chromatography (silica gel 40 g, eluting 50% EtOAc in petroleum ether). LCMS (Method A): 441.6 [M+H]⁺.

### Intermediate 150: N-(4-Chloro-2,3-dihydro-1H-inden-2-yl)-6-((S)-2,2,2-trifluoro-1-(methylamino)ethyl)pyridin-3-amine

The title compound (0.54 g) was prepared in an analogous manner to Intermediate 61 from Intermediate 149 (0.75 g, 2.07 mmol) and LiAlH₄ (1M in THF, 3.0 mL, 3.00 mmol) in THF (7.5 mL) at −15° C. then RT for 1 h. The crude product was purified by flash chromatography (silica gel, eluting 55% EtOAc in petroleum ether). LCMS (Method A): 356.1 [M+H]⁺.

### Intermediate 151: 7-Chloro-6-fluoro-2,3-dihydro-1H-inden-1-ol

To a stirred solution of 7-chloro-6-fluoro-indan-1-one (5.0 g, 27.1 mmol, CAS 881190-95-0) in ethanol (50 mL) at 0° C. was added sodium borohydride (2.06 g, 54.2 mmol) portionwise then the mixture was stirred at RT for 3 h. The mixture was diluted with water and extracted with EtOAc. The organic layer washed with brine, dried over Na₂SO₄, filtered and concentrated under reduced pressure to provide the title compound (4.9 g). ¹H NMR (400 MHz; DMSO-d₆) δ: 7.27-7.22 (m, 2H), 5.27 (d, 1H), 5.15-5.13 (m, 1H), 3.10-3.02 (m, 1H), 2.79-2.72 (m, 1H), 2.33-2.24 (m, 1H), 1.89 (m, 1H).

### Intermediate 152: 4-Chloro-5-fluoro-1H-indene

To a stirred solution of Intermediate 151 (4.9 g, 26.6 mmol) in toluene (200 mL) at 0° C. was added pTSA (1.00 g, 5.26 mmol) then the mixture was stirred at 120° C. for 3 h. The mixture was cooled then diluted with water and extracted with TBDME. The organic layer washed with brine, dried over Na₂SO₄, filtered and concentrated under reduced pressure. The crude product was purified by flash chromatography (silica gel, eluting in petroleum ether) to provide the title compound (3.8 g). ¹H NMR (400 MHz; DMSO-d₆) δ: 7.28-7.17 (m, 2H), 7.01-6.96 (m, 2H), 3.44 (s, 2H).

### Intermediate 153: 4-Chloro-5-fluoro-1,3-dihydro-2H-inden-2-one

Intermediate 152 (3.30 g, 19.6 mmol) was added to a stirred solution of formic acid (13.3 mL) and hydrogen peroxide (30% aqueous, 2.14 mL, 27.4 mmol) at 50° C. then the mixture was stirred at 60° C. for 16 h. Sodium bisulfite (0.94 g) was added and stirred at 60° C. for 1 h. The mixture was concentrated then diluted with water and extracted with DCM. The organic layer washed with brine, dried over Na₂SO₄, filtered and concentrated under reduced pressure. The residue was dissolved in toluene (35 mL) and pTSA added (0.96 g, 5.03 mmol) and stirred at 120° C. for 3 h. The mixture was diluted with water and extracted into EtOAc. The organic layer washed with brine, dried over Na₂SO₄, filtered and concentrated under reduced pressure. The crude product was purified by flash chromatography (silica gel, eluting in petroleum ether) to provide the title compound (0.48 g). LCMS (Method C): 183.0 [M−H]⁻.

### Intermediate 154: tert-Butyl (S)-(1-((1-(5-(benzhydrylamino)pyridin-2-yl)-2,2,2-trifluoroethyl)(methyl)carbamoyl)cyclopropyl)carbamate

The title compound (0.19 g) was prepared in an analogous manner to Intermediate 3 from Intermediate 164 (0.20 g, 0.54 mmol), pyridine (2.7 mL), T3P® (50% in EtOAc, 3.30 mL, 5.34 mmol) and 1-(tert-butoxycarbonylamino)cyclopropanecarboxylic acid (0.16 g, 0.81 mmol, CAS 88950-64-5) at RT for 16 h. Purified by automated flash chromatography (silica gel 25 g, eluting 0-50% EtOAc in cyclohexane). LCMS (Method K): 555.6 [M+H]⁺.

### Intermediate 155: (S)—N-(1-(5-((4,7-Dimethyl-2,3-dihydro-1H-inden-2-yl)amino)pyridin-2-yl)-2,2,2-trifluoroethyl)-N-methylpivalamide

The title compound (55 mg) was prepared in an analogous manner to Intermediate 11 from Intermediate 59 (0.21 g, 0.60 mmol), 4,7-dimethylindan-2-amine (0.10 g, 2.90 mmol, CAS 162752-12-7), Cs₂CO₃ (0.58 g, 1.78 mmol), and RuPhosPdG3 (75 mg, 0.09 mmol) in toluene (2.8 mL) at 110° C. for 14 h. Purified by flash chromatography (silica gel, eluting 15-25% EtOAc in petroleum ether). LCMS (Method J): 434.2 [M+H]⁺.

### Intermediate 156: (S)—N-(4,7-Dimethyl-2,3-dihydro-1H-inden-2-yl)-6-(2,2,2-trifluoro-1-(methylamino)ethyl)pyridin-3-amine

The title compound (29 mg) was prepared in an analogous manner to Intermediate 61 from Intermediate 155 (55 mg, 0.13 mmol) and LiAlH₄ (2M in THF, 0.13 mL, 0.25 mmol) in THF (0.6 mL) at −15° C. for 20 min. LCMS (Method A): 350.3 [M+H]⁺.

### Intermediate 157: N-((1S)-1-(5-((6-Chloro-4-fluoro-2,3-dihydro-1H-inden-2-yl)amino)pyridin-2-yl)-2,2,2-trifluoroethyl)-N-methylpivalamide

The title compound (0.25 g) was prepared in an analogous manner to Intermediate 11 from Intermediate 59 (0.50 g, 1.39 mmol), 6-chloro-4-fluoroindan-2-amine hydrochloride (0.38 g, 1.53 mmol, CAS 2755418-45-0), Cs₂CO₃ (1.36 g, 4.16 mmol), and RuPhosPdG3 (0.12 g, 0.14 mmol) in toluene (10 mL) at 110° C. for 16 h. Purified by flash chromatography (silica gel, eluting 20-25% EtOAc in petroleum ether). LCMS (Method C): 458.5 [M+H]⁺.

### Intermediate 158: N-(6-Chloro-4-fluoro-2,3-dihydro-1H-inden-2-yl)-6-((S)-2,2,2-trifluoro-1-(methylamino)ethyl)pyridin-3-amine

The title compound (0.19 g) was prepared in an analogous manner to Intermediate 61 from Intermediate 157 (0.25 g, 0.45 mmol) and LiAlH₄ (2M in THF, 1.12 mL, 2.24 mmol) in THF (5 mL) at −10° C. for 15 min. LCMS (Method C): 374.5 [M+H]⁺.

### Intermediate 159: N-((1S)-1-(5-((5,6-Difluoro-2,3-dihydro-1H-inden-2-yl)amino)pyridin-2-yl)-2,2,2-trifluoroethyl)-N-methylpivalamide

The title compound (0.25 g) was prepared in an analogous manner to Intermediate 11 from Intermediate 59 (70 mg, 0.20 mmol), 5,6-difluoroindan-2-amine (35 mg, 0.21 mmol, CAS 173996-48-0), Cs₂CO₃ (0.19 g, 0.60 mmol), and RuPhosPdG3 (25 mg, 0.03 mmol) in toluene (0.7 mL) at 110° C. for 14 h. Purified by automated column chromatography (silica gel 25 g, eluting 0-40% EtOAc in hexane). LCMS (Method J): 442.2 [M+H]⁺.

### Intermediate 160: N-(5,6-Difluoro-2,3-dihydro-1H-inden-2-yl)-6-((S)-2,2,2-trifluoro-1-(methylamino)ethyl)pyridin-3-amine

The title compound (10 mg) was prepared in an analogous manner to Intermediate 61 from Intermediate 159 (32 mg, 0.07 mmol) and LiAlH₄ (2M in THF, 0.07 mL, 0.15 mmol) in THF (0.4 mL) at −15° C. for 30 min. Purified by prep-TLC (eluting 30% EtOAc in hexane). LCMS (Method J): 358.1 [M+H]⁺.

### Intermediate 161: N-((1S)-1-(5-((5-Chloro-4-fluoro-2,3-dihydro-1H-inden-2-yl)amino)pyridin-2-yl)-2,2,2-trifluoroethyl)-N-methylpivalamide

The title compound (0.25 g) was prepared in an analogous manner to Intermediate 11 from Intermediate 59 (0.50 g, 1.39 mmol), 5-chloro-4-fluoroindan-2-amine hydrochloride (0.37 g, 1.53 mmol, CAS 2740843-37-0), Cs₂CO₃ (1.36 g, 4.16 mmol), and RuPhosPdG3 (0.12 g, 0.14 mmol) in toluene (10 mL) at 90° C. for 16 h. Purified by automated column chromatography (silica gel, eluting 0-30% EtOAc in hexane). LCMS (Method C): 458.5 [M+H]⁺.

### Intermediate 162: N-(5-Chloro-4-fluoro-2,3-dihydro-1H-inden-2-yl)-6-((S)-2,2,2-trifluoro-1-(methylamino)ethyl)pyridin-3-amine

The title compound (0.20 g) was prepared in an analogous manner to Intermediate 61 from Intermediate 161 (0.25 g, 0.44 mmol) and LiAlH₄ (2M in THF, 0.77 mL, 1.54 mmol) in THF (5 mL) at −10° C. for 15 min. LCMS (Method C): 374.4 [M+H]⁺.

### Intermediate 163: (S)—N-(1-(5-((Diphenylmethylene)amino)pyridin-2-yl)-2,2,2-trifluoroethyl)-N-methylpivalamide

The title compound (5.0 g) was prepared in an analogous manner to Intermediate 26 from Intermediate 59 (4.3 g, 15.7 mmol), benzophenone imine (3.94 mL, 23.5 mmol), Cs₂CO₃ (15.3 g, 47.0 mmol), Pd₂(dba)₃ (1.43 g, 1.57 mmol) and X-Phos (1.49 g, 0.74 mmol) in toluene (40 mL) at 110° C. for 16 h. Purified by flash chromatography (silica gel, eluting 0-100% EtOAc in petroleum ether). LCMS (Method C): 454.6 [M+H]⁺.

### Intermediate 164: (S)—N-Benzhydryl-6-(2,2,2-trifluoro-1-(methylamino)ethyl)pyridin-3-amine

The title compound (4.0 g) was prepared in an analogous manner to Intermediate 61 from Intermediate 163 (5.21 g, 11.0 mmol) and LiAlH₄ (2M in THF, 11.0 mL, 22.1 mmol) in THF (50 mL) at −10° C. for 30 min. LCMS (Method C): 372.4 [M+H]⁺.

### Intermediate 165: (S)—N-(1-(5-(Benzhydrylamino)pyridin-2-yl)-2,2,2-trifluoroethyl)-N-methyltetrahydro-2H-thiopyran-4-carboxamide 1,1-dioxide

The title compound (4.5 g) was prepared in an analogous manner to Intermediate 3 from Intermediate 164 (4.25 g, 10.8 mmol), pyridine (12 mL), T3P® (50% in EtOAc, 32.1 mL, 107 mmol) and tetrahydro-2H-thiopyran-4-carboxylic acid 1,1-dioxide (5.76 g, 32.3 mmol, CAS 64096-87-3) at RT for 16 h. Purified by flash chromatography (silica gel, eluting 10-50% EtOAc in isohexane). LCMS (Method C): 532.6 [M+H]⁺.

### Intermediate 166: (S)—N-(1-(5-aminopyridin-2-yl)-2,2,2-trifluoroethyl)-N-methyltetrahydro-2H-thiopyran-4-carboxamide 1,1-dioxide

To a stirred solution of Intermediate 165 (4.55 g, 2.12 mmol) in MeOH (50 mL), was added palladium on carbon (10%, 2.70 g) and the mixture stirred under a hydrogen atmosphere for 4 h. The mixture was filtered through Celite, washed with EtOAc and concentrated under reduced pressure to provide the title compound (3.0 g). LCMS (Method C): 366.4 [M+H]⁺.

### Intermediate 167: 5-((S)-2,2,2-Trifluoro-1-(methylamino)ethyl)-N-(4-(trifluoromethyl)-2,3-dihydro-1H-inden-2-yl)pyridin-2-amine

The title compound (1.0 g) was prepared in an analogous manner to Intermediate 26 from Intermediate 62 (1.02 g, 3.72 mmol), 4-(trifluoromethyl)-indan-2-amine hydrochloride (1.35 g, 5.58 mmol, CAS 1916506-14-3), sodium t-butoxide (1.07 g, 11.2 mmol), Pd₂(dba)₃ (0.34 g, 0.37 mmol) and X-Phos (0.18 g, 0.37 mmol) in toluene (20 mL) at 110° C. for 16 h. Purified by flash chromatography (silica gel, eluting 0-100% EtOAc in petroleum ether). LCMS (Method C): 390.2 [M+H]⁺.

### Intermediate 168: 4-Chloro-6-fluoro-1,3-dihydro-2H-inden-2-one

The title compound (0.6 g) was prepared in an analogous manner to Intermediate 153 from 7-chloro-5-fluoro-1H-indene (8.0 g, 42.7 mmol, CAS 1215279-93-8) and hydrogen peroxide (30% aqueous, 8.0 mL, 70.6 mmol) in formic acid (37.7 mL) at 60° C. for 16 h, quenching with sodium bisulfite (2.05 g) at 60° C. for 1 h. This was then treated with pTSA (0.47 g, 2.47 mmol) in toluene (100 mL) at 80° C. for 7 h. The crude product was purified by flash chromatography (silica gel, eluting 5% EtOAc in petroleum ether). LCMS (Method A): 183.1 [M−H]⁻.

### Intermediate 169: N-(5-Chloro-2,3-dihydro-1H-inden-2-yl)-5-((S)-2,2,2-trifluoro-1-(methylamino)ethyl)pyridin-2-amine

The title compound (0.32 g) was prepared in an analogous manner to Intermediate 26 from Intermediate 62 (0.50 g, 1.82 mmol), 5-chloroindan-2-amine hydrochloride (0.51 g, 2.18 mmol, CAS 73536-86-4), sodium t-butoxide (0.88 g, 9.11 mmol), Pd₂(dba)₃ (0.17 g, 0.18 mmol) and X-Phos (0.17 g, 0.36 mmol) in toluene (5 mL) at 110° C. for 16 h. Purified by flash chromatography (silica gel, eluting 20-30% EtOAc in petroleum ether). LCMS (Method C): 356.5 [M+H]⁺.

### Intermediate 170: (S)-1-Amino-N-(1-(5-(benzhydrylamino)pyridin-2-yl)-2,2,2-trifluoroethyl)-N-methylcyclopropane-1-carboxamide Dihydrochloride

To a stirred solution of Intermediate 154 (0.19 g, 0.33 mmol) in 1,4-dioxane (17 mL) was added 4M HCl in 1,4-dioxane (1 mL) and stirred at RT for 3 h. The mixture was concentrated under reduced to provide the title compound (0.15 g). LCMS (Method K): 455.2 [M+H]⁺.

### Intermediate 171: 6-Chloro-5-(difluoromethoxy)-2,3-dihydro-1H-inden-1-one

To a stirred solution of 6-chloro-5-hydroxy-2,3-dihydro-1H-inden-1-one (0.60 g, 3.29 mmol, CAS 1260012-31-4) in MeCN (16.5 mL) was added Cs₂CO₃ (2.14 g, 6.57 mmol) and chlorodifluoroacetic acid (0.56 mL, 6.61 mmol) and the mixture was heated at 120° C. for 30 min under microwave irradiation. The mixture was filtered washing with DCM. The filtrate washed with water, brine, dried over Na₂SO₄, filtered and concentrated under reduced pressure to provide the title compound (0.72 g). ¹H NMR (300 MHz; DMSO-d₆) δ: 7.81 (s, 1H), 7.60 (t, 1H), 7.48 (t, 1H) 3.20-3.01 (m, 2H), 2.77-2.60 (m, 2H).

### Intermediate 172: 6-Chloro-5-(difluoromethoxy)-2,3-dihydro-1H-inden-1-ol

The title compound (0.74 g) was prepared in an analogous manner to Intermediate 151 from Intermediate 171 (0.72 g, 0.86 mmol) and sodium borohydride (0.16 g, 1.21 mmol) in THF (14 mL) and MeOH (3.5 mL) at RT for 1.5 h. ¹H NMR (300 MHz; DMSO-d₆) δ: 7.45 (d, 1H), 7.24 (t, 1H), 7.23 (d, 1H), 5.39 (d, 1H), 5.04 (q, 1H), 3.00-2.84 (m, 1H), 2.72 (dt, 1H), 2.42-2.29 (m, 1H), 1.88-1.71 (m, 1H).

### Intermediate 173: 5-Chloro-6-(difluoromethoxy)-1H-indene

The title compound (0.63 mg) was prepared in an analogous manner to Intermediate 152 using Intermediate 172 (0.71 g, 3.02 mmol) and pTSA (0.10 g, 0.60 mmol) and MgSO₄ (0.36 g, 3.01 mmol) in toluene (29 mL) at 80° C. for 1 h. ¹H NMR (400 MHz; CDCl₃) δ: 7.64 (s, 1H), 7.52 (s, 1H), 7.23 (t, 1H), 6.94-6.89 (m, 1H), 6.74 (dt, 1H), 3.48 (s, 2H).

### Intermediate 174: 5-Chloro-6-(difluoromethoxy)-1,3-dihydro-2H-inden-2-one

The title compound (0.32 g) was prepared in an analogous manner to Intermediate 153 from Intermediate 173 (0.63 g, 2.89 mmol) and hydrogen peroxide (35% aqueous, 0.35 mL, 4.05 mmol) in formic acid (1.9 mL) at 60° C. for 16 h, quenching with sodium bisulfite (0.12 g) at 55° C. for 1 h. This was then treated with pTSA (0.12 g, 0.58 mmol) in toluene (4.2 mL) at 115° C. for 2 h. The crude product was purified by automated column chromatography (silica gel, eluting in 4-38% EtOAc in cyclohexane). ¹H NMR (400 MHz; CDCl₃) δ: 7.56 (s, 1H), 7.36 (s, 1H), 7.26 (t, 1H), 3.58 (s, 2H), 3.56 (s, 2H).

### Intermediate 175: Methyl 4-(methylcarbamoyl)cubane-1-carboxylate

To a stirred solution of 4-methoxycarbonylcubane-1-carboxylic acid (50 mg, 0.24 mmol, CAS 24539-28-4) in DCM (1.2 mL) at 0° C. was added oxalyl chloride (25 μL, 0.29 mmol) then the mixture was stirred at RT for 2 h. Methylamine (1M in THF, 1.21 mL, 1.58 mmol) was added then the mixture stirred at RT for 16 h. The mixture was concentrated under reduced pressure to provide the title compound (53 mg). ¹H NMR (300 MHz; DMSO-d₆) δ: 7.61 (br s, 1H), 4.10 (s, 6H), 3.62 (s, 3H), 2.58 (d, 3H).

### Intermediate 176: 4-(Methylcarbamoyl)cubane-1-carboxylic Acid

To a stirred solution of Intermediate 175 (0.11 g, 0.51 mmol) in THF (2 mL) and water (1.3 mL) was added lithium hydroxide (24 mg, 1.00 mmol) and the mixture was stirred at RT for 3 h. The mixture was concentrated under reduced pressure and dissolved in MeOH and treated with Amberlite® (IR 120H form) and filtered to provide the title compound (0.19 g). ¹H NMR (300 MHz; DMSO-d₆) δ: 7.55 (d, 1H), 3.91 (dd, 3H), 3.85-3.75 (m, 3H), 2.57 (dd, 3H)—OH not observed.

### Intermediate 177: (E)-5,6,7-Trifluoro-2-(hydroxyimino)-2,3-dihydro-1H-inden-1-one

To a stirred solution of 5,6,7-trifluoroindan-1-one (2.0 g, 10.5 mmol, CAS 1257844-57-7) in MeOH (20 mL) at 40° C. was added butyl nitrite (1.35 mL, 11.6 mmol) and the mixture was stirred at 40° C. for 16 h. Hydrochloric acid (0.28 mL, 11.6 mmol) was added and the mixture was stirred at 40° C. for 3 h. The mixture was concentrated under reduced pressure then diluted with water and extracted with EtOAc. The organic layer washed with brine, dried over Na₂SO₄, filtered and concentrated under reduced pressure. The crude product was purified by flash chromatography (silica gel, eluting 0-100% EtOAc in hexane) to provide the title compound (1.10 g). LCMS (Method C): 213.9 [M−H]⁻.

### Intermediate 178: 4,5,6-Trifluoro-2,3-dihydro-1H-inden-2-amine Hydrochloride

The title compound (0.42 g) was prepared in an analogous manner to Intermediate 109 from Intermediate 177 (2.2 g, 9.00 mmol) in AcOH (14 mL), sulfuric acid (0.48 mL, 9.00 mmol), palladium on carbon (10%, 1.92 g) under a hydrogen atmosphere for 16 h at 450 psi, acidified with 4M HCl in 1,4-dioxane. LCMS (Method C): 188.2 [M+H]⁺.

### Intermediate 179: N-Methyl-N-((1S)-2,2,2-trifluoro-1-(5-((4,5,6-trifluoro-2,3-dihydro-1H-inden-2-yl)amino)pyridin-2-yl)ethyl)pivalamide

To a stirred solution of Intermediate 178 (0.26 g, 0.88 mmol) in 1,4-dioxane (3 mL) was added Intermediate 59 (0.45 g, 2.31 mmol) and Cs₂CO₃ (0.53 g, 1.61 mmol) and the mixture was degassed with argon for 10 min. Pd-PEPPSI-iHeptCI (39 mg, 0.04 mmol) was added then the mixture was heated at 110° C. for 5 h. The mixture was poured into water and extracted with EtOAc. The organic layer washed with brine, dried over Na₂SO₄, filtered and concentrated under reduced pressure. The crude product was purified by flash chromatography (silica gel, eluting 0-30% EtOAc in hexane) to provide the title compound (0.25 g). LCMS (Method C): 460.6 [M+H]⁺.

### Intermediate 180: 6-((S)-2,2,2-Trifluoro-1-(methylamino)ethyl)-N-(4,5,6-trifluoro-2,3-dihydro-1H-inden-2-yl)pyridin-3-amine

The title compound (0.13 g) was prepared in an analogous manner to Intermediate 61 from Intermediate 179 (0.20 g, 0.34 mmol) and LiAlH₄ (2M in THF, 0.52 mL, 1.03 mmol) in THF (2 mL) at 0° C. for 30 min. LCMS (Method C): 376.5 [M+H]⁺.

### Intermediate 181: 7-(Difluoromethoxy)-1H-indene

The title compound (0.95 g) was prepared in an analogous manner to Intermediate 152 using 4-(difluoromethoxy)-2,3-dihydro-1H-inden-1-ol (0.98 g, 4.90 mmol, CAS 1304084-59-0) and pTSA (0.19 g, 0.98 mmol) in toluene (24 mL) at 80° C. for 3 h. ¹H NMR (300 MHz; CDCl₃) δ: 7.29 (d, 1H), 7.27 (s, 1H), 6.99-6.92 (m, 1H), 6.88 (dt, 1H), 6.63-6.60 (m, 1H), 6.59 (t, 1H), 3.45 (t, 2H).

### Intermediate 182: (S)—N-(1-(5-(Benzhydrylamino)pyridin-2-yl)-2,2,2-trifluoroethyl)-N-methyl-1-(methylsulfonamido)cyclopropane-1-carboxamide

The title compound (0.36 g) was prepared in an analogous manner to Example 169 from Intermediate 170 (0.51 g, 0.97 mmol), triethylamine (0.40 mL, 2.90 mmol) and methane sulfonyl chloride (0.13 g, 1.16 mmol) in DCM (32 mL) at RT for 16 h. Purified by flash chromatography (silica gel, eluting 0-100% EtOAc in hexane). ¹H NMR (300 MHz; DMSO-d₆) δ: 8.15-8.04 (m, 1H), 7.44-7.31 (m, 9H), 7.25 (t, 2H), 7.06 (br s, 1H), 7.00 (d, 2H), 6.16 (dd, 1H), 5.72 (d, 1H), 3.06 (br s, 3H), 2.85 (s, 3H), 1.25-1.05 (m, 4H).

### Intermediate 183: (S)—N-(1-(5-Aminopyridin-2-yl)-2,2,2-trifluoroethyl)-N-methyl-1-(methylsulfonamido)cyclopropane-1-carboxamide

The title compound (0.28 g) was prepared in an analogous manner to Intermediate 206 from Intermediate 182 (0.45 g, 0.84 mmol), formic acid (0.32 mL, 8.40 mmol), and palladium on carbon (10%, 0.25 g) in MeOH (8.4 mL) at 50° C. for 5 h. Purified by automated flash chromatography (12 g silica gel, eluting 0-10% MeOH in DCM). LCMS (Method M): 367.1 [M+H]⁺.

### Intermediate 184: tert-Butyl (S)-(5-chloro-2,3-dihydro-1H-inden-2-yl)carbamate

The title compound (12 g) was prepared in an analogous manner to Intermediate 141 using 5-chloroindan-2-amine hydrochloride (25.0 g, 123 mmol, CAS 73536-86-4), triethylamine (51.2 mL, 368 mmol) and di-tert-butyl dicarbonate (32.1 g, 147 mmol) in DCM (250 mL) at RT for 3 h. Purified by flash chromatography (silica gel, eluting 0-10% EtOAc in isohexane) followed by preparative SFC (Chiralpak®-AD-H, 30×250 mm×5 μm, temp 30° C., Back Pressure 100 bar, flow rate: 100 g/min, 90% CO₂ with 10% 1:1 MeCN/IPA modifier)—title compound Peak 1. LCMS (Method C): 212.2 [M−tBu+H]⁺.

### Intermediate 185: (S)-5-Chloro-2,3-dihydro-1H-inden-2-amine Hydrochloride

To a stirred solution of Intermediate 184 (12.0 g, 44.4 mmol) in 1,4-dioxane (60 mL) was added 4M HCl in 1,4-dioxane (120 mL) and stirred at RT for 3 h. The mixture was concentrated under reduced pressure and triturated in pentane to provide the title compound (10.0 g). LCMS (Method C): 168.2 [M+H]⁺.

### Intermediate 186: N—((S)-1-(5-(((S)-5-Chloro-2,3-dihydro-1H-inden-2-yl)amino)pyridin-2-yl)-2,2,2-trifluoroethyl)-N-methylpivalamide

The title compound (9.0 g) was prepared in an analogous manner to Intermediate 11 from Intermediate 59 (9.0 mg, 25.2 mmol), Intermediate 185 (5.2 g, 25.2 mmol), Cs₂CO₃ (25.7 g, 75.7 mmol), and RuPhosPdG3 (2.11 g, 2.52 mmol) in toluene (180 mL) at 100° C. for 16 h. Purified by flash chromatography (silica gel, eluting 20-25% EtOAc in petroleum ether). LCMS (Method J): 440.5 [M+H]⁺.

### Intermediate 187: N—((S)-5-Chloro-2,3-dihydro-1H-inden-2-yl)-6-((S)-2,2,2-trifluoro-1-(methylamino)ethyl)pyridin-3-amine

The title compound (7.0 g) was prepared in an analogous manner to Intermediate 61 from Intermediate 186 (9.0 g, 18.4 mmol) and LiAlH₄ (2M in THF, 25 mL, 50.0 mmol) in THF (90 mL) at 0° C. for 30 min. ¹H NMR (400 MHz; DMSO-d₆) δ: 7.98 (d, 1H), 7.31-7.19 (m, 4H), 7.01 (dd, 1H), 6.33 (d, 1H), 4.32-4.23 (m, 1H), 4.18-4.08 (m, 1H), 2.84-2.73 (m, 2H), 2.63 (br s, 1H), 2.25-2.19 (m, 4H)—one H obscured by water peak.

### Intermediate 188: tert-Butyl (3-(((S)-1-(5-(((S)-5-chloro-2,3-dihydro-1H-inden-2-yl)amino)pyridin-2-yl)-2,2,2-trifluoroethyl)(methyl)carbamoyl)bicyclo[1.1.1]pentan-1-yl)carbamate

The title compound (0.10 g) was prepared in an analogous manner to Intermediate 3 from Intermediate 187 (60 mg, 0.17 mmol), 3-(tert-butoxycarbonylamino)-bicyclo[1.1.1]pentane-1-carboxylic acid (42 mg, 0.18 mmol, CAS 303752-38-7), pyridine (0.8 mL), T3P® (50% in EtOAc, 0.30 mL, 0.51 mmol) at RT for 16 h. LCMS (Method K): 565.1 [M+H]⁺.

### Intermediate 189: 3-Amino-N—((S)-1-(5-(((S)-5-chloro-2,3-dihydro-1H-inden-2-yl)amino)pyridin-2-yl)-2,2,2-trifluoroethyl)-N-methylbicyclo[1.1.1]pentane-1-carboxamide

To a stirred solution of Intermediate 188 (0.10 g, 0.18 mmol) in 1,4-dioxane (1.4 mL) was added 4M HCl in 1,4-dioxane (0.44 mL) and stirred at RT for 17 h. The mixture was concentrated under reduced pressure and basified with saturated aqueous NaHCO₃ and extracted with diethyl ether. The organics were dried over MgSO₄, filtered and concentrated under reduced pressure, to provide the title compound (77 mg). LCMS (Method K): 465.0 [M+H]⁺.

### Intermediate 190: tert-Butyl (R)-2-(((S)-1-(5-(((S)-5-chloro-2,3-dihydro-1H-inden-2-yl)amino)pyridin-2-yl)-2,2,2-trifluoroethyl)(methyl)carbamoyl)-5-oxopyrrolidine-1-carboxylate

The title compound (14 mg) was prepared in an analogous manner to Intermediate 3 from Intermediate 187 (50 mg, 0.14 mmol), (2R)-1-tert-butoxycarbonyl-5-oxo-pyrrolidine-2-carboxylic acid (40 mg, 0.15 mmol, CAS 160347-90-0), pyridine (0.7 mL), T3P® (50% in EtOAc, 0.25 mL, 0.42 mmol) at RT for 16 h. Purified by preparative HPLC (Gemini NX-C18, 21.2×250 mm×5 μm, flow rate: 30 mL/min, 0.1% aqueous formic acid with MeCN 27% isocratic for 6 min, ramped to 95% over 0.2 min and held for 2.2 min). ¹H NMR (300 MHz; CDCl₃) δ: 8.04 (d, 1H), 7.23-7.13 (m, 4H), 6.92 (dd, 1H), 6.42 (dd, 1H), 5.02 (dd, 1H), 4.39-4.30 (m, 1H), 3.36 (dt, 2H), 3.12 (s, 3H), 2.93-2.81 (m, 2H), 2.80-2.59 (m, 1H), 2.49-2.40 (m, 1H), 2.34-2.18 (m, 1H), 2.00 (s, 1H), 1.95-1.89 (m, 1H), 1.49 (s, 9H).

### Intermediate 191: 7-Bromo-6-chloro-1H-indene

The title compound (1.75 g) was prepared in an analogous manner to Intermediate 152 using 4-bromo-5-chloro-2,3-dihydro-1H-inden-1-ol (1.93 g, 7.81 mmol, CAS 2153981-78-1) and pTSA (0.30 g, 1.56 mmol) in toluene (26 mL) at 80° C. for 1 h. ¹H NMR (300 MHz; CDCl₃) δ: 7.38 (d, 1H), 7.26 (d, 1H), 6.88 (dt, 1H), 6.62 (dt, 1H), 3.43 (s, 2H).

### Intermediate 192: 4-Bromo-5-chloro-1,3-dihydro-2H-inden-2-one

The title compound (0.79 g) was prepared in an analogous manner to Intermediate 153 from Intermediate 191 (1.75 g, 7.60 mmol) and hydrogen peroxide (35% aqueous, 0.93 mL, 10.6 mmol) in formic acid (5.0 mL) at 60° C. for 16 h, quenching with sodium bisulfite (0.32 g) at 55° C. for 1 h. This was then treated with pTSA (0.29 g, 1.52 mmol) in toluene (9.5 mL) at 80° C. for 2 h. The crude product was purified by automated column chromatography (silica gel 120 g, eluting in 0-90% EtOAc in hexane). ¹H NMR (300 MHz; CDCl₃) δ: 7.38 (d, 1H), 7.20 (d, 1H), 3.65 (s, 2H), 3.59 (s, 2H).

### Intermediate 193: 5-Chloro-4-cyclopropyl-1,3-dihydro-2H-inden-2-one

To a stirred solution of Intermediate 192 (0.25 g, 0.87 mmol) in toluene (4.8 mL) and water (0.8 ml) was added potassium cyclopropyltrifluoroborate (0.26 g, 2.00 mmol, CAS 1065010-87-8) and the mixture was degassed with argon for 10 min. Pd(dppf)Cl₂ (64 mg, 0.10 mmol) and K₃PO₄ (0.55 g, 3.00 mmol) were added then the mixture was heated at 90° C. for 16 h. The mixture was poured into water and extracted with EtOAc. The organic layer washed with water, dried over Na₂SO₄, filtered and concentrated under reduced pressure. The crude product was purified by automated column chromatography (silica gel 80 g, eluting 0-20% EtOAc in cyclohexane) to provide the title compound (0.65 g). ¹H NMR (300 MHz; DMSO-d₆) δ: 7.32 (d, 1H), 7.20 (d, 1H), 3.65 (s, 2H), 3.53 (s, 2H), 1.88-1.74 (m, 1H), 1.05-0.88 (m, 2H), 0.72-0.65 (m, 2H).

### Intermediate 194: (R)-6-Oxo-1-(trimethylsilyl)piperidine-2-carboxylic Acid

To a stirred solution of (R)-6-oxopiperidine-2-carboxylic acid (0.10 g, 0.70 mmol, CAS 72002-30-3) and triethylamine (0.11 mL, 0.77 mmol) in toluene (3.9 mL) was added chlorotrimethylsilane (0.09 mL, 0.77 mmol) and the mixture was heated at 110° C. for 16 h. The mixture was concentrated under reduced pressure to provide the title compound (0.15 g). ¹H NMR (300 MHz; CDCl₃) δ: 11.99 (d, 1H), 4.06 (dd, 1H), 2.50-2.18 (m, 3H), 1.98-1.86 (m, 1H), 1.83-1.60 (m, 2H), 0.05 (s, 9H).

### Intermediate 195: 5-Bromo-6-fluoro-1H-indene

The title compound (2.55 g) was prepared in an analogous manner to Intermediate 152 using 6-bromo-5-fluoro-2,3-dihydro-1H-inden-1-ol (2.52 g, 10.9 mmol, CAS 2089649-48-7) and pTSA (0.31 g, 1.09 mmol) in toluene (57 mL) at 90° C. for 2 h. ¹H NMR (300 MHz; DMSO-d₆) δ: 7.76 (dd, 1H), 7.42 (d, 1H), 6.93-6.89 (m, 1H), 6.81-6.74 (m, 1H), 3.43 (s, 2H).

### Intermediate 196: 5-Bromo-6-fluoro-1,3-dihydro-2H-inden-2-one

The title compound (0.79 g) was prepared in an analogous manner to Intermediate 153 from Intermediate 195 (2.25 g, 10.6 mmol) and hydrogen peroxide (35% aqueous, 1.29 mL, 14.8 mmol) in formic acid (7.0 mL) at 60° C. for 16 h, quenching with sodium bisulfite (0.44 g) at 55° C. for 1 h. This was then treated with pTSA (0.40 g, 2.11 mmol) in toluene (53 mL) at 80° C. for 1.5 h. ¹H NMR (300 MHz; DMSO-d₆) δ: 7.65 (d, 1H), 7.35 (d, 1H), 3.54 (s, 4H).

### Intermediate 197: 5-Chloro-4-(trifluoromethyl)-2,3-dihydro-1H-inden-1-ol

The title compound (2.2 g) was prepared in an analogous manner to Intermediate 151 from 5-chloro-4-(trifluoromethyl)indan-1-one (2.70 g, 8.98 mmol, CAS 1273661-11-2) and sodium borohydride (0.51 g, 13.5 mmol) in ethanol (30 mL) at RT for 3 h. Purified by flash chromatography (silica gel, eluting 0-10% EtOAc in petroleum ether). LCMS (Method C): 219.2 [M−H₂O+H]⁺.

### Intermediate 198: 6-Chloro-7-(trifluoromethyl)-1H-indene

The title compound (2.20 g) was prepared in an analogous manner to Intermediate 152 using Intermediate 197 (2.70 g, 6.73 mmol) and pTSA (0.26 g, 1.34 mmol) in toluene (30 mL) at 90° C. for 3 h. ¹H NMR (400 MHz; CDCl₃) δ: 7.43 (dd, 2H), 6.85 (d, 1H), 6.66 (dd, 1H), 3.65-3.62 (m, 2H).

### Intermediate 199: 5-Chloro-4-(trifluoromethyl)-1,3-dihydro-2H-inden-2-one

The title compound (0.35 g) was prepared in an analogous manner to Intermediate 153 from Intermediate 198 (1.10 g, 5.03 mmol) and hydrogen peroxide (30% aqueous, 0.55 mL, 7.04 mmol) in formic acid (3.4 mL) at 60° C. for 16 h, quenching with sodium bisulfite (0.28 g) at 60° C. for 2 h. This was then treated with pTSA (0.18 g, 0.95 mmol) in toluene (25 mL) at 90° C. for 3 h. LCMS (Method C): 235.1 [M+H]⁺.

### Intermediate 200: tert-Butyl (S)-2-(((S)-1-(5-(((S)-5-chloro-2,3-dihydro-1H-inden-2-yl)amino)pyridin-2-yl)-2,2,2-trifluoroethyl)(methyl)carbamoyl)-5-oxopyrrolidine-1-carboxylate

The title compound (20 mg) was prepared in an analogous manner to Intermediate 3 from Intermediate 187 (70 mg, 0.20 mmol), (2S)-1-(tert-butoxycarbonyl)-5-oxo-pyrrolidine-2-carboxylic acid (80 mg, 0.29 mmol, CAS 53100-44-0), pyridine (1.0 mL), T3P® (50% in EtOAc, 1.17 mL, 2.95 mmol) at RT for 16 h. Purified by preparative HPLC (X Bridge C18, 19×150 mm×5 μm, flow rate: 30 mL/min, 0.05% aqueous ammonia with MeCN 10% to 60% over 6 min, held at 60% for 7 min, ramped to 95% over 0.2 min and held for 1.8 min). LCMS (Method K): 567.4 [M+H]⁺.

### Intermediate 201: (S)—N—((S)-1-(5-(Benzhydrylamino)pyridin-2-yl)-2,2,2-trifluoroethyl)-N-methyl-5-oxopyrrolidine-3-carboxamide

The title compound (0.39 g) was prepared in an analogous manner to Intermediate 3 from Intermediate 164 (0.40 g, 1.03 mmol), (3S)-5-oxopyrrolidine-3-carboxylic acid (0.14 g, 1.08 mmol, CAS 30948-17-5), pyridine (5.2 mL), T3P® (50% in EtOAc, 3.0 mL, 4.90 mmol) at RT for 40 h. Purified by flash chromatography (silica gel, eluting 0-10% MeOH in DCM). LCMS (Method K): 483.2 [M+H]⁺.

### Intermediate 202: (S)—N—((S)-1-(5-Aminopyridin-2-yl)-2,2,2-trifluoroethyl)-N-methyl-5-oxopyrrolidine-3-carboxamide

To a stirred solution of Intermediate 201 (0.39 g, 0.81 mmol) in MeOH (4 mL), was added formic acid (0.09 mL, 2.41 mmol), ammonium formate (0.31 g, 4.84 mmol) and palladium on carbon (10%, 0.17 g) and the mixture stirred at RT for 3 h. The mixture was concentrated under reduced pressure, diluted with saturated aqueous NaHCO₃ and extracted with DCM. The organic layer washed with brine, dried over Na₂SO₄, filtered and concentrated under reduced pressure. The crude product was purified by flash chromatography (silica gel, eluting 0-20% MeOH in DCM) to provide the title compound (0.14 g). LCMS (Method M): 317.2 [M+H]⁺.

### Intermediate 203: 6-Chloro-7-fluoro-1H-indene

The title compound (4.00 g) was prepared in an analogous manner to Intermediate 152 using 4-fluoro-5-chloro-2,3-dihydro-1H-inden-1-ol (5.00 g, 26.8 mmol, CAS 1597134-92-3) and pTSA (1.02 g, 5.37 mmol) in toluene (134 mL) at 80° C. for 1 h. ¹H NMR (300 MHz; CDCl₃) δ: 7.30 (dd, 1H), 7.11 (d, 1H), 6.85-6.80 (m, 1H), 6.61-6.58 (m, 1H), 3.48 (t, 2H).

### Intermediate 204: 5-Chloro-4-fluoro-1,3-dihydro-2H-inden-2-one

The title compound (0.52 g) was prepared in an analogous manner to Intermediate 153 from Intermediate 203 (2.0 g, 11.6 mmol) and hydrogen peroxide (35% aqueous, 1.43 mL, 16.3 mmol) in formic acid (7.7 mL) at 60° C. for 16 h, quenching with sodium bisulfite (0.48 g) at 55° C. for 1 h. This was then treated with pTSA (0.44 g, 0.20 mmol) in toluene (60 mL) at 90° C. for 2 h. Purified by flash chromatography (silica gel, eluting 0-20% EtOAc in cyclohexane). ¹H NMR (300 MHz; CDCl₃) δ: 7.32 (dt, 1H), 7.05 (d, 1H), 3.60 (s, 2H), 3.57 (s, 2H).

### Intermediate 205: (S)—N—((S)-1-(5-(Benzhydrylamino)pyridin-2-yl)-2,2,2-trifluoroethyl)-N-methyl-6-oxopiperidine-3-carboxamide

The title compound (1.12 g) was prepared in an analogous manner to Intermediate 3 from Intermediate 164 (1.11 g, 2.89 mmol), (3S)-6-oxopiperidine-3-carboxylic acid (0.50 g, 1.20 mmol, CAS 1426408-56-1), pyridine (15.8 mL), T3P® (50% in EtOAc, 8.8 mL, 14.4 mmol) at RT for 72 h. Purified by flash chromatography (silica gel, eluting 0-20% MeOH in DCM). LCMS (Method K): 497.3 [M+H]⁺.

### Intermediate 206: (S)—N—((S)-1-(5-Aminopyridin-2-yl)-2,2,2-trifluoroethyl)-N-methyl-6-oxopiperidine-3-carboxamide

To a stirred solution of Intermediate 205 (1.11 g, 1.79 mmol) in MeOH (18 mL), was added formic acid (0.68 mL, 17.9 mmol), and palladium on carbon (10%, 0.57 g) and the mixture stirred at 50° C. for 2 h. The mixture was concentrated under reduced pressure, diluted with saturated aqueous NaHCO₃ and extracted with DCM. The organic layer washed with brine, dried over Na₂SO₄, filtered and concentrated under reduced pressure. The crude product was purified by flash chromatography (silica gel, eluting 0-10% MeOH in DCM) to provide the title compound (0.47 g). LCMS (Method K): 331.1 [M+H]⁺.

### Intermediate 207: tert-Butyl (S)-(3-((1-(5-(benzhydrylamino)pyridin-2-yl)-2,2,2-trifluoroethyl)(methyl)carbamoyl)bicyclo[1.1.1]pentan-1-yl)carbamate

The title compound (0.60 g) was prepared in an analogous manner to Intermediate 3 from Intermediate 164 (0.40 g, 1.08 mmol), 3-(tert-butoxycarbonylamino)bicyclo[1.1.1]pentane-1-carboxylic acid (0.30 g, 1.30 mmol, CAS 303752-38-7), pyridine (6.0 mL), T3P® (50% in EtOAc, 2.0 mL, 3.27 mmol) at RT for 16 h. Purified by flash chromatography (silica gel, eluting 0-50% EtOAc in cyclohexane). LCMS (Method L): 581.3 [M+H]⁺.

### Intermediate 208: (S)-3-Amino-N-(1-(5-(benzhydrylamino)pyridin-2-yl)-2,2,2-trifluoroethyl)-N-methylbicyclo[1.1.1]pentane-1-carboxamide

A solution of Intermediate 207 (0.60 g, 0.98 mmol) in TFA (0.60 mL) and DCM (9.8 mL) was stirred at RT for 6 h. The mixture was poured into saturated aqueous NaHCO₃ and extracted with DCM. The organics were dried over Na₂SO₄, filtered and concentrated under reduced pressure to provide the title compound (0.50 g). LCMS (Method K): 481.2 [M+H]⁺.

### Intermediate 209: (S)—N-(1-(5-(Benzhydrylamino)pyridin-2-yl)-2,2,2-trifluoroethyl)-N-methyl-3-(methylsulfonamido)bicyclo[1.1.1]pentane-1-carboxamide

To a solution of Intermediate 208 (0.50 g, 0.94 mmol) and triethylamine (0.39 mL, 2.80 mmol) in DCM (9.4 mL) at 0° C. was added methanesulfonyl chloride (0.09 mL, 1.12 mmol) then the mixture was stirred at RT for 3 h. The mixture was poured into water and extracted with DCM. The organics were dried over Na₂SO₄, filtered and concentrated under reduced pressure. The crude product was purified by flash chromatography (silica gel, eluting 0-3% MeOH in DCM) to provide the title compound (0.41 g). LCMS (Method K): 559.1 [M+H]⁺.

### Intermediate 210: (S)—N-(1-(5-Aminopyridin-2-yl)-2,2,2-trifluoroethyl)-N-methyl-3-(methylsulfonamido)bicyclo[1.1.1]pentane-1-carboxamide

The title compound (0.26 g) was prepared in an analogous manner to Intermediate 206 from Intermediate 209 (0.41 g, 0.73 mmol), formic acid (0.27 mL, 7.16 mmol), and palladium on carbon (10%, 0.23 g) in MeOH (6.9 mL) at 50° C. for 2 h. LCMS (Method K): 393.0 [M+H]⁺.

### Intermediate 211: 5-(Difluoromethoxy)-1,3-dihydro-2H-inden-2-one

The title compound (0.18 g) was prepared in an analogous manner to Intermediate 153 from Intermediate 247 (0.80 g, 3.07 mmol) and hydrogen peroxide (35% aqueous, 0.38 mL, 4.32 mmol) in formic acid (2.0 mL) at 60° C. for 16 h, quenching with sodium bisulfite (0.40 g) at 55° C. for 1 h. This was then treated with pTSA (0.12 g, 0.62 mmol) in toluene (18 mL) at 90° C. for 2 h. Purified by flash chromatography (silica gel, eluting 0-40% EtOAc in cyclohexane). ¹H NMR (300 MHz; CDCl₃) δ: 7.30 (d, 1H), 7.11-7.02 (d, 2H), 6.51 (t, 1H) 3.58 (s, 2H), 3.55 (s, 2H).

### Intermediate 212: (S)—N-(1-(5-(Benzhydrylamino)pyridin-2-yl)-2,2,2-trifluoroethyl)-N-methyl-1-(methylsulfonyl)piperidine-4-carboxamide

The title compound (0.60 g) was prepared in an analogous manner to Intermediate 3 from Intermediate 164 (0.20 g, 0.52 mmol), 1-(methylsulfonyl)piperidine-4-carboxylic acid (0.14 g, 0.68 mmol, CAS 280772-00-1), pyridine (2.6 mL), T3P® (50% in MeTHF, 0.96 mL, 1.57 mmol) at RT for 16 h. Purified by flash chromatography (silica gel, eluting 0-50% EtOAc in cyclohexane). ¹H NMR (300 MHz; DMSO-d₆) δ: 8.12-8.03 (m, 1H), 7.45-7.31 (m, 8H), 7.30-7.12 (m, 2H), 7.11-6.93 (m, 3H), 6.35 (dd, 1H), 5.77-5.67 (m, 1H), 3.64-3.47 (m, 2H), 2.99 (s, 3H), 2.86 (s, 3H), 2.81-2.67 (m, 3H), 1.92-1.47 (m, 4H).

### Intermediate 213: (S)—N-(1-(5-Aminopyridin-2-yl)-2,2,2-trifluoroethyl)-N-methyl-1-(methylsulfonyl)piperidine-4-carboxamide

The title compound (92 mg) was prepared in an analogous manner to Intermediate 206 from Intermediate 212 (0.22 g, 0.39 mmol), formic acid (0.22 g, 4.71 mmol), and palladium on carbon (10%, 0.13 g) in MeOH (1.3 mL) at 50° C. for 3 h. Purified by flash chromatography (silica gel, eluting 0-10% MeOH in DCM). ¹H NMR (300 MHz; DMSO-d₆) δ: 7.99-7.90 (m, 1H), 7.19-6.90 (m, 2H), 6.36 (dd, 1H), 5.65-5.53 (m, 2H), 3.67-3.52 (m, 2H), 2.99 (s, 3H), 2.89 (s, 3H), 2.83-2.69 (m, 2H), 1.95-1.46 (m, 5H).

### Intermediate 214: (S)—N-(1-(6-Bromopyridin-3-yl)-2,2,2-trifluoroethyl)-N-methylpivalamide

To a stirred solution of Intermediate 62 (0.20 g, 0.74 mmol) and triethylamine (0.51 mL, 3.68 mmol) in DCM (4 mL) at 10° C. was added pivaloyl chloride (0.54 mL, 4.42 mmol) and the mixture was stirred at RT for 16 h. The mixture was poured into water and extracted with DCM. The organics were washed with saturated aqueous NaHCO₃, brine, dried over Na₂SO₄, filtered and concentrated under reduced pressure. The crude product was purified by flash chromatography (silica gel, eluting 0-8% EtOAc in hexane) to provide the title compound (0.20 g). LCMS (Method C): 353.3 [M+H]⁺.

### Intermediate 215: N-((1S)-1-(6-((5-Chloro-4-fluoro-2,3-dihydro-1H-inden-2-yl)amino)pyridin-3-yl)-2,2,2-trifluoroethyl)-N-methylpivalamide

The title compound (92 mg) was prepared in an analogous manner to Intermediate 179 from Intermediate 214 (0.26 g, 0.88 mmol), 5-chloro-4-fluoro-indan-2-amine hydrochloride (0.45 g, 2.31 mmol, CAS 2740843-37-0), Cs₂CO₃ (0.79 g, 2.42 mmol) and Pd-PEPPSI-iHeptCI (55 mg, 0.08 mmol) in 1,4-dioxane (6 mL) at 100° C. for 16 h. Purified by flash chromatography (silica gel, eluting 0-100% EtOAc in petroleum ether) to provide the title compound (0.11 g). LCMS (Method C): 458.3 [M+H]⁺.

### Intermediate 216: N-(5-Chloro-4-fluoro-2,3-dihydro-1H-inden-2-yl)-5-((S)-2,2,2-trifluoro-1-(methylamino)ethyl)pyridin-2-amine

The title compound (0.15 g) was prepared in an analogous manner to Intermediate 61 from Intermediate 215 (0.16 g, 0.31 mmol) and LiAlH₄ (2M in THF, 0.92 mL, 1.84 mmol) in THF (5 mL) at 0° C. for 30 min. LCMS (Method C): 374.2 [M+H]⁺.

### Intermediate 217: 5-(Difluoromethoxy)-1a,6a-dihydro-6H-indeno[1,2-b]oxirene

To a stirred solution of Intermediate 181 (0.75 g, 3.29 mmol) in DCM (16.5 mL) was added NaHCO₃ (0.41 g, 4.90 mmol) and MCPBA (1.22 g, 4.94 mmol) the mixture was stirred at RT for 16 h. The mixture was poured into water and extracted with DCM. The organics were washed with water, brine, dried over Na₂SO₄, filtered and concentrated under reduced pressure. The crude product was purified by automated column chromatography (silica gel 25 g, 0-10% EtOAc in cyclohexane) to provide the title compound (0.54 g). ¹H NMR (300 MHz; CDCl₃) δ: 7.38 (dd, 1H), 7.21 (dt, 1H), 7.04 (dt, 1H), 6.50 (t, 1H), 4.29 (dd, 1H), 4.16 (t, 1H), 3.27 (dd, 1H), 2.97 (dd, 1H).

### Intermediate 218: 4-(Difluoromethoxy)-1,3-dihydro-2H-inden-2-one

To a stirred solution of Intermediate 217 (0.53 g, 2.67 mmol) in toluene (13 mL) was added silica gel (1.2 g) and the mixture was stirred at reflux for 1 h. The mixture was filtered and concentrated under reduced pressure to provide the title compound (0.40 g). ¹H NMR (300 MHz; CDCl₃) δ: 7.33-7.27 (m, 1H), 7.18 (dd, 1H), 7.07-7.03 (m, 1H), 6.56 (t, 1H), 3.60 (s, 2H), 3.57 (s, 2H).

### Intermediate 219: tert-Butyl (S)-3-((1-(5-(Benzhydrylamino)pyridin-2-yl)-2,2,2-trifluoroethyl)(methyl)carbamoyl)azetidine-1-carboxylate

The title compound (0.43 g) was prepared in an analogous manner to Intermediate 3 from Intermediate 164 (0.40 g, 1.08 mmol), 1-(tert-butoxycarbonyl)azetidine-3-carboxylic acid (0.17 g, 0.85 mmol, CAS 142253-55-2), pyridine (4.1 mL), T3P® (50% in MeTHF, 1.99 mL, 3.23 mmol) at RT for 2 h. ¹H NMR (400 MHz; DMSO-d₆) δ: 8.07 (d, 1H), 7.44-7.22 (m, 10H), 7.11 (d, 1H), 7.05-6.97 (m, 2H), 6.31 (dd, 1H), 5.71 (d, 1H), 4.09-3.74 (m, 5H), 2.78 (s, 3H), 1.39 (d, 9H).

### Intermediate 220: (S)—N-(1-(5-(Benzhydrylamino)pyridin-2-yl)-2,2,2-trifluoroethyl)-N-methylazetidine-3-carboxamide

A solution of Intermediate 219 (0.42 g, 0.75 mmol) in TFA (0.58 mL) and DCM (5 mL) was stirred at RT for 17 h. The mixture was poured into saturated aqueous NaHCO₃ and extracted with DCM. The organics were dried over MgSO₄, filtered and concentrated under reduced pressure to provide the title compound (0.30 g). ¹H NMR (400 MHz; DMSO-d₆) δ: 8.06 (d, 1H), 7.44-7.32 (m, 10H), 7.28-6.96 (m, 5H), 6.32 (dd, 1H), 5.71 (d, 1H), 3.79 (q, 1H), 3.64 (q, 1H), 3.54 (t, 1H), 3.46 (t, 1H), 2.74 (s, 3H).

### Intermediate 221: (S)—N³-(1-(5-(Benzhydrylamino)pyridin-2-yl)-2,2,2-trifluoroethyl)-N¹,N³-dimethylazetidine-1,3-dicarboxamide

To a stirred solution of Intermediate 220 (0.28 g, 0.62 mmol) and triethylamine (0.26 mL, 1.85 mmol) in DCM (3.4 mL) at 0° C. was added methylcarbamoyl chloride (53.5 μL, 0.68 mmol) and the mixture was stirred at RT for 2 h. The mixture was poured into water and extracted with DCM. The organics were dried over MgSO₄ filtered and concentrated under reduced pressure to provide the title compound (0.30 g). LCMS (Method K): 512.5 [M+H]⁺.

### Intermediate 222: (S)—N³-(1-(5-aminopyridin-2-yl)-2,2,2-trifluoroethyl)-N¹,N³-dimethylazetidine-1,3-dicarboxamide

The title compound (0.15 g) was prepared in an analogous manner to Intermediate 206 from Intermediate 221 (0.28 g, 0.47 mmol), formic acid (0.17 mL, 4.61 mmol), and palladium on carbon (10%, 0.15 g) in MeOH (5 mL) at 50° C. for 2 h. LCMS (Method L): 346.2 [M+H]⁺.

### Intermediate 223: (S)—N-(1-(5-(Benzhydrylamino)pyridin-2-yl)-2,2,2-trifluoroethyl)-N-methyl-6-oxo-5-azaspiro[3.4]octane-2-carboxamide

The title compound (0.34 g) was prepared in an analogous manner to Intermediate 3 from Intermediate 164 (0.30 g, 0.81 mmol), 6-oxo-5-azaspiro[3.4]octane-2-carboxylic acid (0.24 g, 1.05 mmol, CAS 2166848-89-9), pyridine (8.1 mL), T3P® (50% in MeTHF, 1.50 mL, 2.45 mmol) at RT for 16 h. Purified by flash chromatography (silica gel, eluting 0-50% EtOAc in cyclohexane). LCMS (Method K): 523.3 [M+H]⁺.

### Intermediate 224: (S)—N-(1-(5-Aminopyridin-2-yl)-2,2,2-trifluoroethyl)-N-methyl-6-oxo-5-azaspiro[3.4]octane-2-carboxamide

The title compound (0.23 g) was prepared in an analogous manner to Intermediate 206 from Intermediate 223 (0.39 g, 0.70 mmol), formic acid (0.27 mL, 7.04 mmol), and palladium on carbon (10%, 0.23 g) in MeOH (7 mL) at 50° C. for 2 h. LCMS (Method L): 357.2 [M+H]⁺.

### Intermediate 225: N-((1S)-1-(6-((4,5-Dichloro-2,3-dihydro-1H-inden-2-yl)amino)pyridin-3-yl)-2,2,2-trifluoroethyl)-N-methylpivalamide

The title compound (0.35 g) was prepared in an analogous manner to Intermediate 26 from Intermediate 214 (0.50 g, 1.26 mmol), 4,5-dichloro-2,3-dihydro-1H-inden-2-amine hydrochloride (0.40 g, 1.26 mmol, CAS 2755418-27-8), Cs₂CO₃ (1.23 g, 3.78 mmol), Pd₂(dba)₃ (0.12 g, 0.13 mmol) and Xantphos (0.15 g, 0.25 mmol) in 1,4-dioxane (20 mL) at 100° C. for 16 h. Purified by flash chromatography (silica gel, eluting 0-30% EtOAc in petroleum ether). LCMS (Method C): 474.4 [M+H]⁺.

### Intermediate 226: N-(4,5-Dichloro-2,3-dihydro-1H-inden-2-yl)-5-((S)-2,2,2-trifluoro-1-(methylamino)ethyl)pyridin-2-amine

The title compound (0.14 g) was prepared in an analogous manner to Intermediate 61 from Intermediate 225 (0.36 g, 0.45 mmol) and LiAlH₄ (2M in THF, 0.45 mL, 0.90 mmol) in THF (10 mL) at 0° C. for 15 min. LCMS (Method C): 390.3 [M+H]⁺.

### Intermediate 227: N-((1S)-1-(5-((4,5-Dichloro-2,3-dihydro-1H-inden-2-yl)amino)pyridin-2-yl)-2,2,2-trifluoroethyl)-N-methylpivalamide

The title compound (1.0 g) was prepared in an analogous manner to Example 265 from Intermediate 59 (0.95 g, 3.12 mmol), 4,5-dichloro-1,3-dihydro-2H-inden-2-one (0.99 g, 4.68 mmol, CAS 69392-70-7), AcOH (0.18 mL, 3.13 mmol) in MeOH (24 mL) at 60° C. for 6 h then sodium cyanoborohydride (0.39 g, 6.24 mmol) at RT for 8 h. Purified by flash chromatography (silica gel, eluting 0-15% MeOH in DCM). LCMS (Method C): 474.3 [M+H]⁺.

### Intermediate 228: N-(4,5-Dichloro-2,3-dihydro-1H-inden-2-yl)-6-((S)-2,2,2-trifluoro-1-(methylamino)ethyl)pyridin-3-amine

The title compound (0.85 g) was prepared in an analogous manner to Intermediate 61 from Intermediate 227 (1.1 g, 2.09 mmol) and LiAlH₄ (2M in THF, 2.09 mL, 4.17 mmol) in THF (10 mL) at 0° C. for 15 min. LCMS (Method C): 390.2 [M+H]⁺.

### Intermediate 229: N-((1S)-1-(5-((4-bromo-5-chloro-2,3-dihydro-1H-inden-2-yl)amino)pyridin-2-yl)-2,2,2-trifluoroethyl)-N-methyltetrahydro-2H-thiopyran-4-carboxamide 1,1-dioxide

The title compound (0.59 g) was prepared in an analogous manner to Example 265 from Intermediate 166 (0.50 g, 1.37 mmol), Intermediate 192 (0.67 g, 2.74 mmol), AcOH (41 mg, 0.68 mmol) in MeOH (6.8 mL) at 50° C. for 3 h then sodium cyanoborohydride (1.72 g, 27.4 mmol) at RT for 90 h. Purified by flash chromatography (silica gel, eluting 0-100% EtOAc in hexane). LCMS (Method J): 596.0 [M+H]⁺.

### Intermediate 230: Methyl (1,3-trans)-3-(((S)-1-(5-(benzhydrylamino)pyridin-2-yl)-2,2,2-trifluoroethyl)(methyl)carbamoyl)cyclobutane-1-carboxylate

The title compound (0.29 g) was prepared in an analogous manner to Intermediate 3 from Intermediate 164 (0.30 g, 0.81 mmol), trans-3-(methylcarbamoyl)cyclobutane-1-carboxylate (0.19 g, 1.21 mmol, CAS 1401103-71-6), pyridine (2.7 mL), T3P® (50% in MeTHF, 2.47 mL, 4.04 mmol) at RT for 16 h. Purified by automated flash chromatography (silica gel 80 g, eluting 0-100% EtOAc in hexane). LCMS (Method K): 512.2 [M+H]⁺.

### Intermediate 231: Methyl (1,3-trans)-3-(((S)-1-(5-aminopyridin-2-yl)-2,2,2-trifluoroethyl)(methyl)carbamoyl)cyclobutane-1-carboxylate

The title compound (0.11 g) was prepared in an analogous manner to Intermediate 206 from Intermediate 230 (0.23 g, 0.45 mmol), formic acid (0.17 mL, 4.50 mmol), and palladium on carbon (10%, 0.14 g) in MeOH (4.5 mL) at 50° C. for 3 h. ¹H NMR (400 MHz; DMSO-d₆) δ: 7.93 (d, 1H), 7.06 (d, 1H), 6.94 (dd, 1H), 6.34 (dd, 1H), 5.57 (s, 2H), 3.63 (s, 3H), 3.59-3.42 (m, 1H), 3.17-3.00 (m, 1H), 2.80 (s, 3H), 2.43-2.34 (m, 4H).

### Intermediate 232: Methyl (1,3-trans)-3-(((1S)-1-(5-((4,5-dichloro-2,3-dihydro-1H-inden-2-yl)amino)pyridin-2-yl)-2,2,2-trifluoroethyl)(methyl)carbamoyl)cyclobutane-1-carboxylate

The title compound (62 mg) was prepared in an analogous manner to Example 265 from Intermediate 231 (0.14 g, 0.39 mmol), 4,5-dichloro-1,3-dihydro-2H-inden-2-one (0.16 g, 0.78 mmol, CAS 69392-70-7), AcOH (26 mg, 0.43 mmol) in MeOH (2 mL) at 50° C. for 6 h then sodium cyanoborohydride (98 mg, 1.56 mmol) at RT for 8 h. Purified by flash chromatography (silica gel, eluting 0-100% EtOAc in hexane). LCMS (Method J): 529.9 [M+H]⁺.

### Intermediate 233: (1,3-trans)-3-(((1S)-1-(5-((4,5-Dichloro-2,3-dihydro-1H-inden-2-yl)amino)pyridin-2-yl)-2,2,2-trifluoroethyl)(methyl)carbamoyl)cyclobutane-1-carboxylic Acid

To a stirred solution of Intermediate 232 (62 mg, 0.12 mmol) in THF (1 mL) and MeOH (0.5 mL) was added lithium hydroxide (8.4 mg, 0.35 mmol) and the mixture was stirred at RT for 16 h. The mixture diluted with 1M aqueous sodium dihydrogenphosphate until pH 4 then extracted with DCM. The organic layer was dried over Na₂SO₄, filtered and concentrated under reduced pressure to provide the title compound (41 mg). LCMS (Method J): 515.9 [M+H]⁺.

### Intermediate 234: 2-(tert-Butoxy)-N-((1S)-1-(5-((5-chloro-4-fluoro-2,3-dihydro-1H-inden-2-yl)amino)pyridin-2-yl)-2,2,2-trifluoroethyl)-N-methylacetamide

The title compound (0.15 g) was prepared in an analogous manner to Intermediate 3 from Intermediate 162 (0.25 g, 0.56 mmol), 2-tert-butoxyacetic acid (0.22 g, 1.66 mmol, CAS 13211-32-0), pyridine (3.0 mL), T3P® (50% in EtOAc, 1.76 mg, 5.55 mmol) at RT for 16 h. Purified by flash chromatography (silica gel, eluting 0-100% EtOAc in petroleum ether). LCMS (Method J): 488.4 [M+H]⁺.

### Intermediate 235: (S)—N-(1-(5-(Benzhydrylamino)pyridin-2-yl)-2,2,2-trifluoroethyl)-N,N′-dimethylcyclopropane-1,1-dicarboxamide

The title compound (0.33 g) was prepared in an analogous manner to Intermediate 3 from Intermediate 164 (0.25 g, 0.67 mmol), 1-(methylcarbamoyl)cyclopropanecarboxylic acid (0.10 g, 0.71 mmol, CAS 1250809-34-7), pyridine (2.7 mL), T3P® (50% in MeTHF, 1.65 mL, 2.69 mmol) at RT for 17 h. ¹H NMR (400 MHz; DMSO-d₆) δ: 8.08 (d, 1H), 7.63 (d, 1H), 7.44-7.39 (m, 5H), 7.37-7.32 (m, 4H), 7.28-7.22 (m, 2H), 7.16 (d, 1H), 7.03-7.01 (m, 1H), 6.25 (dd, 1H), 5.73 (d, 1H), 2.85 (s, 3H), 2.57 (d, 3H), 1.38-1.30 (m, 1H), 1.27-1.20 (m, 1H), 1.16-1.03 (m, 2H).

### Intermediate 236: (S)—N-(1-(5-Aminopyridin-2-yl)-2,2,2-trifluoroethyl)-N,N′-dimethylcyclopropane-1,1-dicarboxamide

The title compound (0.23 g) was prepared in an analogous manner to Intermediate 206 from Intermediate 235 (0.31 g, 0.62 mmol), formic acid (0.23 mL, 6.19 mmol), and palladium on carbon (10%, 0.20 g) in MeOH (3.9 mL) at 50° C. for 2 h. LCMS (Method K): 331.2 [M+H]⁺.

### Intermediate 237: tert-Butyl (S)-3-((1-(5-aminopyridin-2-yl)-2,2,2-trifluoroethyl)(methyl)carbamoyl)azetidine-1-carboxylate

The title compound (0.40 g) was prepared in an analogous manner to Intermediate 206 from Intermediate 219 (0.50 g, 0.90 mmol), formic acid (0.34 mL, 8.93 mmol), and palladium on carbon (10%, 0.29 g) in MeOH (5.9 mL) at 50° C. for 2 h. LCMS (Method K): 389.1 [M+H]⁺.

### Intermediate 238: tert-Butyl 3-(((1S)-1-(5-((5-chloro-4-fluoro-2,3-dihydro-1H-inden-2-yl)amino)pyridin-2-yl)-2,2,2-trifluoroethyl)(methyl)carbamoyl)azetidine-1-carboxylate

The title compound (0.14 g) was prepared in an analogous manner to Example 265 from Intermediate 237 (0.14 g, 0.39 mmol), Intermediate 204 (0.20 g, 0.98 mmol), AcOH (0.14 mL, 0.25 mmol) in MeOH (6.2 mL) at 50° C. for 2 h then sodium cyanoborohydride (0.16 g, 2.47 mmol) at RT for 2 h. Purified by flash chromatography (silica gel, eluting 20-100% EtOAc in hexane). LCMS (Method K): 557.1 [M+H]⁺.

### Intermediate 239: N-((1S)-1-(5-((5-Chloro-4-fluoro-2,3-dihydro-1H-inden-2-yl)amino)pyridin-2-yl)-2,2,2-trifluoroethyl)-N-methylazetidine-3-carboxamide

A solution of Intermediate 238 (0.35 g, 0.54 mmol) in TFA (0.17 mL) and DCM (1.3 mL) was stirred at RT for 17 h. The mixture was poured into saturated aqueous NaHCO₃ and extracted into DCM. The organic layer was dried over MgSO₄, filtered and concentrated under reduced pressure to provide the title compound (0.10 g). LCMS (Method L): 457.2 [M+H]⁺.

### Intermediate 240: (1,3-trans)-3-((tert-Butyldimethylsilyl)oxy)-N-((1S)-1-(5-((5-chloro-4-fluoro-2,3-dihydro-1H-inden-2-yl)amino)pyridin-2-yl)-2,2,2-trifluoroethyl)-N-methylcyclobutane-1-carboxamide

The title compound (0.21 g) was prepared in an analogous manner to Intermediate 3 from Intermediate 162 (0.20 g, 0.48 mmol), (1,3-trans)-3-((tert-butyldimethylsilyl)oxy)cyclobutane-1-carboxylic acid (0.11 g, 0.48 mmol, CAS 1431285-79-8), triethylamine (0.67 mL, 4.82 mmol), T3P® (50% in MeTHF, 1.53 mL, 2.41 mmol) at RT for 4 h. LCMS (Method C): 586.6 [M+H]⁺.

### Intermediate 241: (S)—N-(1-(5-(Benzhydrylamino)pyridin-2-yl)-2,2,2-trifluoroethyl)-N-methylthietane-3-carboxamide 1,1-dioxide

The title compound (0.15 g) was prepared in an analogous manner to Intermediate 3 from Intermediate 164 (0.12 g, 0.31 mmol), thietane-3-carboxylic acid 1,1-dioxide (56 mg, 0.37 mmol, CAS 13129-21-0), pyridine (3.1 mL), T3P® (50% in MeTHF, 0.57 mL, 0.93 mmol) at RT for 17 h. Purified by automated flash chromatography (25 g silica gel, eluting 0-50% EtOAc in hexane). LCMS (Method L): 504.3 [M+H]⁺.

### Intermediate 242: (S)—N-(1-(5-Aminopyridin-2-yl)-2,2,2-trifluoroethyl)-N-methylthietane-3-carboxamide 1,1-dioxide

The title compound (60 mg) was prepared in an analogous manner to Intermediate 206 from Intermediate 241 (0.15 g, 0.27 mmol), formic acid (0.10 mL, 2.65 mmol), and palladium on carbon (10%, 85 mg) in MeOH (2.7 mL) at 50° C. for 1 h. Purified by automated flash chromatography (40 g silica gel, eluting 0-10% MeOH in DCM). LCMS (Method K): 337.8 [M+H]⁺.

### Intermediate 243: 1-(2-(tert-Butoxy)acetyl)-N-((1S)-1-(5-((4,5-dichloro-2,3-dihydro-1H-inden-2-yl)amino)pyridin-2-yl)-2,2,2-trifluoroethyl)-N-methylazetidine-3-carboxamide

The title compound (0.15 g) was prepared in an analogous manner to Intermediate 3 from Intermediate 228 (0.21 g, 0.49 mmol), 1-(2-tert-butoxyacetyl)azetidine-3-carboxylic acid (0.17 g, 0.73 mmol, CAS 1564594-27-9), pyridine (0.2 mL), T3P® (50% in MeTHF, 2.92 mL, 4.90 mmol) at RT for 2 h. Purified by flash chromatography (silica gel, eluting 0-80% EtOAc in hexane). LCMS (Method C): 587.5 [M+H]⁺.

### Intermediate 244: (1,3-cis)-3-((tert-Butyldimethylsilyl)oxy)-N-((1S)-1-(5-((5-chloro-4-fluoro-2,3-dihydro-1H-inden-2-yl)amino)pyridin-2-yl)-2,2,2-trifluoroethyl)-N-methylcyclobutane-1-carboxamide

The title compound (0.17 g) was prepared in an analogous manner to Intermediate 3 from Intermediate 162 (0.20 g, 0.48 mmol), (1,3-cis)-3-((tert-butyldimethylsilyl)oxy)cyclobutane-1-carboxylic acid (0.44 g, 1.90 mmol, CAS 1431285-80-1), triethylamine (0.93 mL, 6.67 mmol), T3P® (50% in MeTHF, 1.92 mL, 3.33 mmol) in DCM (0.5 mL) at RT for 16 h. Purified by flash chromatography (silica gel, eluting 0-30% EtOAc in petroleum ether). LCMS (Method C): 586.9 [M+H]⁺.

### Intermediate 245: tert-Butyl (5S)-5-(((1S)-1-(6-((5-chloro-4-fluoro-2,3-dihydro-1H-inden-2-yl)amino)pyridin-3-yl)-2,2,2-trifluoroethyl)(methyl)carbamoyl)-2-oxopiperidine-1-carboxylate

The title compound (0.12 g) was prepared in an analogous manner to Intermediate 3 from Intermediate 216 (0.10 g, 0.26 mmol), (3S)-1-tert-butoxycarbonyl-6-oxo-piperidine-3-carboxylic acid (0.31 g, 1.28 mmol, CAS 1629681-71-5), pyridine (0.2 mL), T3P® (50% in MeTHF, 1.59 mL, 2.57 mmol) at RT for 16 h. Purified by flash chromatography (silica gel, eluting 50-70% EtOAc in hexane). LCMS (Method A): 499.3 [M−Boc+H]⁺.

### Intermediate 246: 5-(Difluoromethoxy)-2,3-dihydro-1H-inden-1-ol

The title compound (0.82 g) was prepared in an analogous manner to Intermediate 151 from 5-(difluoromethoxy)indan-1-one (0.87 g, 4.39 mmol, CAS 1273599-48-6) and sodium borohydride (0.18 g, 4.84 mmol) in MeOH (4.4 mL) and THF (18 mL) at RT for 1 h. 1H NMR (300 MHz; CDCl₃) δ: 7.43-7.33 (m, 1H), 7.04-6.92 (m, 2H), 6.49 (t, 1H), 5.23 (dd, 1H), 3.18-2.96 (m, 1H), 2.82 (dt, 1H), 2.61-2.42 (m, 1H), 2.04-1.92 (m, 1H), 1.66 (br s, 1H).

### Intermediate 247: 7-Bromo-6-chloro-1H-indene

The title compound (0.80 g) was prepared in an analogous manner to Intermediate 152 using Intermediate 246 (0.82 g, 4.07 mmol) and pTSA (0.15 g, 0.81 mmol) in toluene (20 mL) at 80° C. for 0.5 h. ¹H NMR (300 MHz; CDCl₃) δ: 7.35 (d, 1H), 7.17 (d, 1H), 7.05 (dd, 1H), 6.88-6.82 (m, 1H), 6.57 (dt, 1H), 6.50 (t, 1H), 3.42-3.40 (m, 2H).

### SYNTHESIS OF EXAMPLES

### Example 1: N-((1S)-1-(4-((1,1-Dimethyl-2,3-dihydro-1H-inden-2-yl)amino)phenyl)-2,2,2-trifluoroethyl)-N-methyltetrahydro-2H-thiopyran-4-carboxamide 1,1-dioxide—Isomer 1

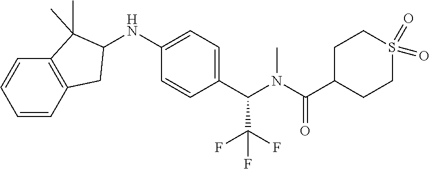 [SMILES: CN(C(=O)C1CCS(=O)(=O)CC1)[C@@H](c1ccc(NC2Cc3ccccc3C2(C)C)cc1)C(F)(F)F]

### Example 2: N-((1S)-1-(4-((1,1-Dimethyl-2,3-dihydro-1H-inden-2-yl)amino)phenyl)-2,2,2-trifluoroethyl)-N-methyltetrahydro-2H-thiopyran-4-carboxamide 1,1-dioxide—Isomer 2

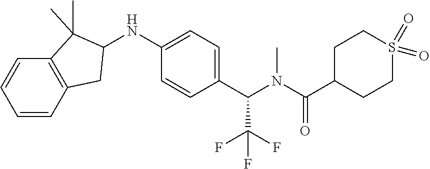 [SMILES: CN(C(=O)C1CCS(=O)(=O)CC1)[C@@H](c1ccc(NC2Cc3ccccc3C2(C)C)cc1)C(F)(F)F]

The title compounds were prepared in an analogous manner to Intermediate 11 from Intermediate 3 (0.5 g, 1.17 mmol), 1,1-dimethyl-2,3-dihydro-1H-inden-2-amine hydrochloride (0.23 g, 1.17 mmol, CAS 74413-86-8), Cs₂CO₃ (1.52 g, 4.68 mmol), tBuBrettphosPdG3 (0.20 g, 0.23 mmol) and RuPhos (0.22 g, 0.47 mmol) in 1,4-dioxane (10 mL) at 120° C. for 1 h. Purified by flash chromatography (silica gel, eluting 45-50% EtOAc in petroleum ether) and preparative HPLC (X Bridge C18, 19×250 mm×5 μm, flow rate: 17 mL/min, 10 mM aqueous NH₄HCO₃ with MeCN 30% to 65% over 6 min, held at 65% for 8 min then ramped to 100% over 0.1 min and held for 2.4 min). Chiral purification was carried out by preparative SFC (Chiralcel-OJ-H, 20×250 mm×5 μm, temp 30° C., Back Pressure 100 bar, flow rate: 60 g/min, 70% CO₂ with 30% 1:1 MeCN/MeOH modifier) to provide Example 1 (56 mg) as Peak 1 and Example 2 (50 mg) as Peak 2. Example 1: ¹H NMR (400 MHz; DMSO-d₆) δ: 7.21-7.12 (m, 6H), 6.78 (d, 2H), 5.94-5.83 (m, 2H), 3.99 (q, 1H), 3.67-3.51 (m, 4H), 3.20-3.06 (m, 5H), 2.77-2.68 (d, 1H), 2.11-1.95 (m, 4H), 1.32 (s, 3H), 1.09 (s, 3H). LCMS (Method A): 509.4 [M+H]⁺. Example 2: ¹H NMR (400 MHz; DMSO-d₆) δ: 7.20-7.12 (m, 4H), 7.50 (d, 2H), 6.79 (d, 2H), 6.34 (dd, 1H), 6.01-5.94 (m, 1H), 3.99 (q, 1H), 3.28-3.08 (m, 6H), 2.89 (s, 3H), 2.73 (dd, 1H), 2.11-1.95 (m, 4H), 1.31 (s, 3H), 1.09 (s, 3H). LCMS (Method A): 509.4 [M+H]⁺.

### Example 3: (S)—N-(1-(4-((2,3-Dihydro-1H-inden-2-yl)amino)phenyl)-2,2,2-trifluoroethyl)-N-methyltetrahydro-2H-thiopyran-4-carboxamide 1,1-dioxide

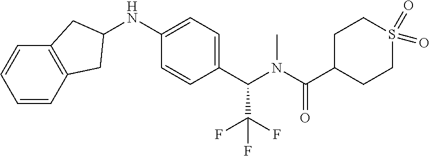 [SMILES: CN(C(=O)C1CCS(=O)(=O)CC1)[C@@H](c1ccc(NC2Cc3ccccc3C2)cc1)C(F)(F)F]

The title compound (40 mg) was prepared in an analogous manner to Intermediate 11 from Intermediate 3 (0.2 g, 0.47 mmol), 2,3-dihydro-1H-inden-2-amine hydrochloride (79 mg, 0.47 mmol, CAS 2975-41-9), Cs₂CO₃ (0.61 g, 1.87 mmol), tBuBrettphosPdG3 (80 mg, 0.01 mmol) and RuPhos (87 mg, 0.19 mmol) in 1,4-dioxane (10 mL) at 120° C. for 1 h. Purified by preparative HPLC (X-Select C18, 19×250 mm×5 μm, flow rate: 17 mL/min, 10 mM aqueous NH₄HCO₃ with MeCN 30% to 65% over 8 min, held at 65% for 7 min then ramped to 98% over 0.1 min and held for 2.9 min). ¹H NMR (400 MHz; DMSO-d₆) δ: 7.28-7.21 (m, 2H), 7.17-7.14 (m, 2H), 7.07 (d, 2H), 6.66 (d, 2H), 6.35 (dd, 1H), 6.26-6.21 (m, 1H), 4.23-4.18 (m, 1H), 3.23-3.08 (m, 7H), 2.88 (s, 3H), 2.79 (dd, 2H), 2.11-1.95 (m, 4H). LCMS (Method D): 481.3 [M+H]⁺.

### Example 4: N-((1S)-1-(4-((1,1-Dimethyl-2,3-dihydro-1H-inden-2-yl)amino)phenyl)-2,2,2-trifluoroethyl)-N-methyloxazole-5-carboxamide—Isomer 1

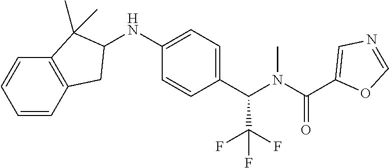 [SMILES: CN(C(=O)c1cnco1)[C@@H](c1ccc(NC2Cc3ccccc3C2(C)C)cc1)C(F)(F)F]

### Example 5: N-((1S)-1-(4-((1,1-Dimethyl-2,3-dihydro-1H-inden-2-yl)amino)phenyl)-2,2,2-trifluoroethyl)-N-methyloxazole-5-carboxamide—Isomer 2

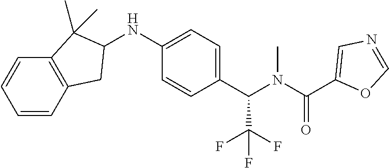 [SMILES: CN(C(=O)c1cnco1)[C@@H](c1ccc(NC2Cc3ccccc3C2(C)C)cc1)C(F)(F)F]

The title compounds were prepared in an analogous manner to Intermediate 11 from Intermediate 4 (0.38 g, 0.96 mmol), 1,1-dimethyl-2,3-dihydro-1H-inden-2-amine hydrochloride (0.23 g, 1.15 mmol, CAS 74413-86-8), Cs₂CO₃ (1.25 g, 3.84 mmol), tBuBrettphosPdG3 (0.16 g, 0.19 mmol) and RuPhos (0.18 g, 0.38 mmol) in 1,4-dioxane (7 mL) at 120° C. for 1 h. Purified by preparative HPLC (X Bridge C18, 19×250 mm×5 μm, flow rate: 17 mL/min, 5 mM aqueous NH₄HCO₃ with MeCN 35% to 75% over 28 min, then ramped to 98% over 0.1 min and held for 2.4 min). Chiral purification was carried out by preparative SFC (Chiralcel OJ-H, 30×250 mm×5 μm, temp 30° C., Back Pressure 100 bar, flow rate: 100 g/min, 70% CO₂ with 30% MeOH modifier) to provide Example 4 (18 mg) as Peak 1 and Example 5 (22 mg) as Peak 2. Example 4: ¹H NMR (400 MHz; DMSO-d₆) δ: 8.59 (s, 1H), 7.90 (br s, 1H), 7.22-7.15 (m, 6H), 6.82 (d, 2H), 6.40 (br s, 1H), 6.01 (d, 1H), 4.01 (t, 1H), 3.17 (dd, 1H), 3.07 (br s, 3H), 2.74 (dd, 1H), 1.32 (s, 3H), 1.09 (s, 3H). LCMS (Method A): 444.2 [M+H]⁺. Example 5: ¹H NMR (400 MHz; DMSO-d₆) δ: 8.64 (s, 1H), 7.92 (br s, 1H), 7.21-7.13 (m, 6H), 6.81 (d, 2H), 6.40 (br s, 1H), 6.01 (d, 1H), 4.01 (dd, 1H), 3.17 (dd, 1H), 3.05 (br s, 3H), 2.74 (dd, 1H), 1.31 (s, 3H), 1.09 (s, 3H). LCMS (Method A): 444.2 [M+H]⁺.

### Example 6: 1-Acetyl-N-((1S)-1-(4-((1,1-dimethyl-2,3-dihydro-1H-inden-2-yl)amino)phenyl)-2,2,2-trifluoroethyl)-N-methylazetidine-3-carboxamide—Isomer 1

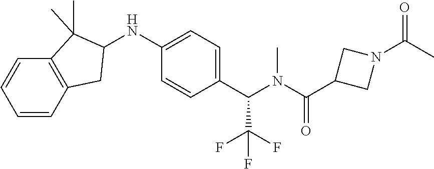 [SMILES: CC(=O)N1CC(C(=O)N(C)[C@@H](c2ccc(NC3Cc4ccccc4C3(C)C)cc2)C(F)(F)F)C1]

### Example 7: 1-Acetyl-N-((1S)-1-(4-((1,1-dimethyl-2,3-dihydro-1H-inden-2-yl)amino)phenyl)-2,2,2-trifluoroethyl)-N-methylazetidine-3-carboxamide—Isomer 2

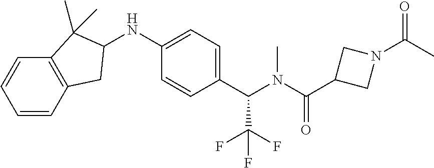 [SMILES: CC(=O)N1CC(C(=O)N(C)[C@@H](c2ccc(NC3Cc4ccccc4C3(C)C)cc2)C(F)(F)F)C1]

The title compounds were prepared in an analogous manner to Intermediate 11 from Intermediate 5 (0.40 g, 1.03 mmol), 1,1-dimethyl-2,3-dihydro-1H-inden-2-amine hydrochloride (0.20 g, 1.03 mmol, CAS 74413-86-8), Cs₂CO₃ (1.04 g, 3.09 mmol), tBuBrettphosPdG3 (44 mg, 0.05 mmol) and RuPhos (48 mg, 0.10 mmol) in 1,4-dioxane (7 mL) at 120° C. for 2 h. Purified by preparative HPLC (X Bridge C18, 19×250 mm×5 μm, flow rate: 17 mL/min, 5 mM aqueous NH₄HCO₃ with MeCN 10% to 50% over 0.1 min, to 67% over 16.9 min, ramped to 98% over 0.1 min and held for 2.9 min). Chiral purification was carried out by preparative SFC (Chiralcel OJ-H, 30×250 mm×5 μm, temp 30° C., Back Pressure 100 bar, flow rate: 90 g/min, 90% CO₂ with 10% MeOH/MeCN 1:1 modifier) to provide Example 6 (18 mg) as Peak 1 and Example 7 (22 mg) as Peak 2. Example 6: ¹H NMR (400 MHz; DMSO-d₆) δ: 7.22-7.05-7.22 (m, 6H), 6.79 (d, 2H), 6.33 (dd, 1H), 5.96 (d, 1H), 4.36-4.16 (m, 2H), 4.10-3.97 (m, 2H), 3.92-3.80 (m, 2H), 3.16 (dd, 1H), 2.76-2.71 (m, 4H), 1.76 (d, 3H), 1.32 (s, 3H), 1.09 (s, 3H). LCMS (Method A): 474.3 [M+H]⁺. Example 7: ¹H NMR (400 MHz; DMSO-d₆) δ: 7.22-7.05 (m, 6H), 6.79 (d, 2H), 6.33 (dd, 1H), 5.96 (d, 1H), 4.36-4.16 (m, 2H), 4.10-3.97 (m, 2H), 3.95-3.81 (m, 2H), 3.17 (dd, 1H), 2.76-2.71 (m, 4H), 1.76 (d, 3H), 1.31 (s, 3H), 1.09 (s, 3H). LCMS (Method A): 474.3 [M+H]⁺.

### Example 8: N-Methyl-N—((S)-2,2,2-trifluoro-1-(4-(((1,2-cis)-1-methyl-2,3-dihydro-1H-inden-2-yl)amino)phenyl)ethyl)tetrahydro-2H-thiopyran-4-carboxamide 1,1-dioxide

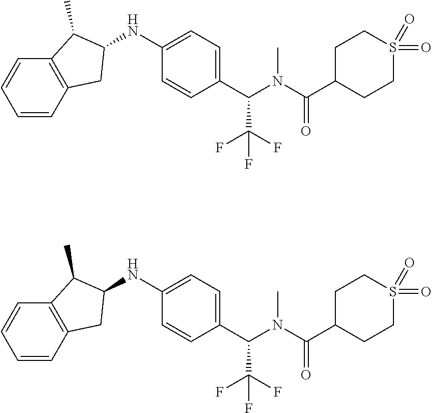 [SMILES: C[C@H]1c2ccccc2C[C@H]1Nc1ccc([C@H](N(C)C(=O)C2CCS(=O)(=O)CC2)C(F)(F)F)cc1 ; C[C@@H]1c2ccccc2C[C@@H]1Nc1ccc([C@H](N(C)C(=O)C2CCS(=O)(=O)CC2)C(F)(F)F)cc1]

### Example 9: N-Methyl-N—((S)-2,2,2-trifluoro-1-(4-(((1,2-trans)-1-methyl-2,3-dihydro-1H-inden-2-yl)amino)phenyl)ethyl)tetrahydro-2H-thiopyran-4-carboxamide 1,1-dioxide

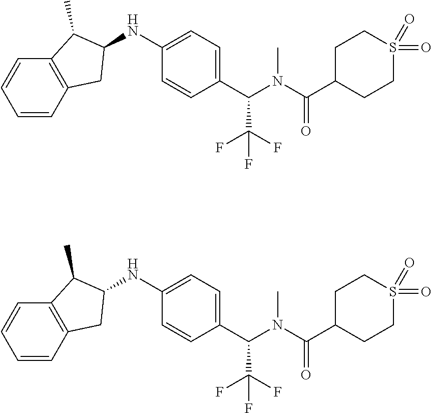 [SMILES: C[C@H]1c2ccccc2C[C@@H]1Nc1ccc([C@H](N(C)C(=O)C2CCS(=O)(=O)CC2)C(F)(F)F)cc1 ; C[C@@H]1c2ccccc2C[C@H]1Nc1ccc([C@H](N(C)C(=O)C2CCS(=O)(=O)CC2)C(F)(F)F)cc1]

The title compounds were prepared in an analogous manner to Intermediate 11 from Intermediate 3 (0.74 g, 1.74 mmol), Intermediate 6 (0.32 g, 1.74 mmol), Cs₂CO₃ (2.27 g, 6.97 mmol), tBuBrettphosPdG3 (0.30 g, 0.05 mmol) and RuPhos (0.33 g, 0.70 mmol) in 1,4-dioxane (10 mL) at 120° C. for 2 h. Purified by preparative HPLC (X Bridge C18, 19×250 mm×5 μm, flow rate: 17 mL/min, 5 mM aqueous NH₄HCO₃ with MeCN 35% to 75% over 28 min, ramped to 98% over 0.1 min and held for 2.4 min). Chiral purification was carried out by preparative SFC (Chiralcel IE, 30×250 mm×5 μm, temp 30° C., Back Pressure 100 bar, flow rate: 110 g/min, 67% CO₂ with 33% IPA modifier) to provide Example 8 (36 mg) as Peak 1 and Example 9 (10 mg) as Peak 2. Example 8: ¹H NMR (400 MHz; DMSO-d₆) δ: 7.26-7.11 (m, 4H), 7.07 (d, 2H), 6.77-6.67 (m, 2H), 6.35 (dd, 1H), 6.28-6.13 (m, 1H), 4.27-4.17 (m, 1H), 3.50-3.06 (m, 7H), 2.94-2.85 (m, 4H), 2.11-1.96 (m, 4H), 0.98 (d, 3H). LCMS (Method A): 495.3 [M+H]⁺. Example 9: ¹H NMR (400 MHz; DMSO-d₆) δ: 7.24-7.13 (m, 4H), 7.07 (d, 2H), 6.74-6.67 (m, 2H), 6.35 (dd, 1H), 6.28-6.23 (m, 1H), 3.76-3.69 (m, 1H), 3.41-3.15 (m, 7H), 2.89 (s, 3H), 2.67-2.55 (m, 1H), 2.11-1.96 (m, 4H), 1.30 (d, 3H). LCMS (Method A): 495.3 [M+H]⁺.

### Example 10: N-Methyl-N—((S)-2,2,2-trifluoro-1-(4-(((1,2-trans)-1-methoxy-2,3-dihydro-1H-inden-2-yl)amino)phenyl)ethyl)tetrahydro-2H-thiopyran-4-carboxamide 1,1-dioxide—Isomer 1

### Example 11: N-Methyl-N—((S)-2,2,2-trifluoro-1-(4-(((1,2-trans)-1-methoxy-2,3-dihydro-1H-inden-2-yl)amino)phenyl)ethyl)tetrahydro-2H-thiopyran-4-carboxamide 1,1-dioxide—Isomer 2

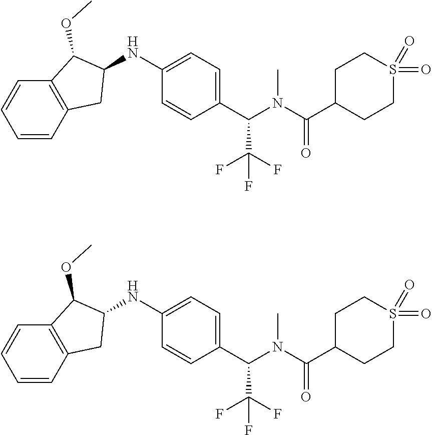 [SMILES: CO[C@H]1c2ccccc2C[C@@H]1Nc1ccc([C@H](N(C)C(=O)C2CCS(=O)(=O)CC2)C(F)(F)F)cc1 ; CO[C@@H]1c2ccccc2C[C@H]1Nc1ccc([C@H](N(C)C(=O)C2CCS(=O)(=O)CC2)C(F)(F)F)cc1]

The title compounds were prepared in an analogous manner to Intermediate 11 from Intermediate 3 (0.23 g, 0.54 mmol), 1-methoxy-2,3-dihydro-1H-inden-2-amine hydrochloride (0.13 g, 0.64 mmol, CAS 2702474-20-0), Cs₂CO₃ (0.70 g, 2.16 mmol), tBuBrettphosPdG3 (23 mg, 0.03 mmol) and RuPhos (25 mg, 0.05 mmol) in 1,4-dioxane (5 mL) at 120° C. for 1 h. Purified by preparative HPLC (X-Select C18, 19×250 mm×5 μm, flow rate: 17 mL/min, 5 mM aqueous NH₄HCO₃ with MeCN 10% to 45% over 0.1 min, then to 61% over 15.9 min, ramped to 95% over 0.1 min and held for 0.1 min). Diastereomer purification was carried out by preparative SFC (Chiralcel OD-H, 30×250 mm×5 μm, temp 30° C., Back Pressure 100 bar, flow rate: 110 g/min, 70% CO₂ with 30% MeOH modifier) to provide Example 10 (32 mg) as Peak 1 and Example 11 (30 mg) as Peak 2. Example 10: ¹H NMR (400 MHz; DMSO-d₆) δ: 7.39 (d, 1H), 7.32-7.16 (m, 3H), 7.09 (d, 2H), 6.75-6.67 (m, 2H), 6.36 (dd, 1H), 6.32-6.24 (m, 1H), 4.66 (d, 1H), 4.10-4.02 (m, 1H), 3.46-3.32 (m, 4H), 3.24-3.06 (m, 5H), 2.89 (s, 3H), 2.70-2.64 (m, 1H), 2.11-1.96 (m, 4H). LCMS (Method A): 511.2 [M+H]⁺. Example 11: ¹H NMR (400 MHz; DMSO-d₆) δ: 7.39 (d, 1H), 7.32-7.16 (m, 3H), 7.11 (d, 2H), 6.75-6.67 (m, 2H), 6.36 (dd, 1H), 6.32-6.24 (br m, 1H), 4.66 (d, 1H), 4.10-4.02 (m, 1H), 3.46-3.32 (m, 4H), 3.24-3.06 (m, 5H), 2.90 (s, 3H), 2.71-2.64 (m, 1H), 2.11-1.96 (m, 4H). LCMS (Method A): 511.2 [M+H]⁺.

### Example 12: N—((S)-1-(4-(((S)-1,1-Dimethyl-2,3-dihydro-1H-inden-2-yl)amino)phenyl)-2,2,2-trifluoroethyl)-N-methylmorpholine-4-carboxamide

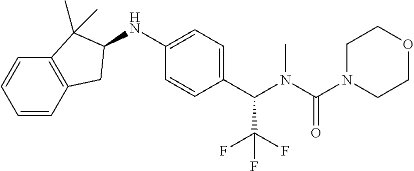 [SMILES: CN(C(=O)N1CCOCC1)[C@@H](c1ccc(N[C@H]2Cc3ccccc3C2(C)C)cc1)C(F)(F)F]

### Example 13: N—((S)-1-(4-(((R)-1,1-Dimethyl-2,3-dihydro-1H-inden-2-yl)amino)phenyl)-2,2,2-trifluoroethyl)-N-methylmorpholine-4-carboxamide

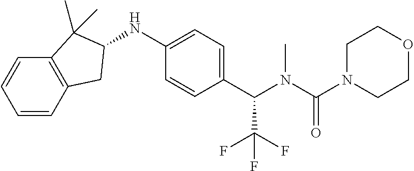 [SMILES: CN(C(=O)N1CCOCC1)[C@@H](c1ccc(N[C@@H]2Cc3ccccc3C2(C)C)cc1)C(F)(F)F]

The title compound were prepared in an analogous manner to Intermediate 11 from Intermediate 7 (0.40 g, 1.05 mmol), 1,1-dimethyl-2,3-dihydro-1H-inden-2-amine hydrochloride (0.20 g, 1.05 mmol, CAS 74413-86-8), Cs₂CO₃ (1.24 g, 3.15 mmol), tBuBrettphosPdG3 (45 mg, 0.05 mmol) and RuPhos (49 mg, 0.1 mmol) in 1,4-dioxane (5 mL) at 120° C. for 2 h. Purified by preparative HPLC (X-Select CSH C18, 19×250 mm×5 μm, flow rate: 17 mL/min, 5 mM aqueous NH₄HCO₃ with MeCN 10% to 50% over 0.1 min, then 78% over 18.9 min, ramped to 95% over 0.1 min and held for 2 min). Chiral purification was carried out by preparative SFC (Chiralcel OJ-H, 30×250 mm×5 μm, temp 30° C., Back Pressure 100 bar, flow rate: 100 g/min, 75% CO₂ with 25% MeOH modifier) to provide Example 12 (75 mg) as Peak 1 and Example 13 (52 mg) as Peak 2. Example 12: ¹H NMR (400 MHz; DMSO-d₆) δ: 7.22-7.11 (m, 6H), 6.78 (d, 2H), 5.93-5.83 (m, 2H), 3.99 (q, 1H), 3.67-3.52 (m, 4H), 3.24-3.11 (m, 5H), 2.77-2.67 (m, 4H), 1.32 (s, 3H), 1.09 (s, 3H). LCMS (Method E): 462.3 [M+H]⁺. Example 13: ¹H NMR (400 MHz; DMSO-d₆) δ: 7.22-7.11 (m, 6H), 6.79 (d, 2H), 5.93-5.83 (m, 2H), 3.99 (q, 1H), 3.67-3.52 (m, 4H), 3.24-3.11 (m, 5H), 2.77-2.67 (m, 4H), 1.32 (s, 3H), 1.09 (s, 3H). LCMS (Method E): 462.2 [M+H]⁺. Example 13 chirality confirmed by crystal structure.

### Example 14: N-((1S)-1-(4-((1,1-Dimethyl-2,3-dihydro-1H-inden-2-yl)amino)phenyl)-2,2,2-trifluoroethyl)-N-methylthiomorpholine-4-carboxamide 1,1-dioxide—Isomer 1

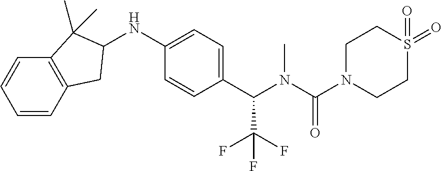 [SMILES: CN(C(=O)N1CCS(=O)(=O)CC1)[C@@H](c1ccc(NC2Cc3ccccc3C2(C)C)cc1)C(F)(F)F]

### Example 15: N-((1S)-1-(4-((1,1-Dimethyl-2,3-dihydro-1H-inden-2-yl)amino)phenyl)-2,2,2-trifluoroethyl)-N-methylthiomorpholine-4-carboxamide 1,1-dioxide—Isomer 2

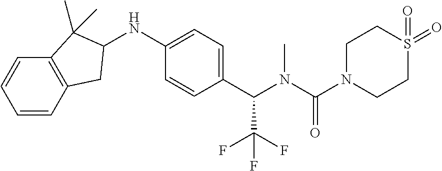 [SMILES: CN(C(=O)N1CCS(=O)(=O)CC1)[C@@H](c1ccc(NC2Cc3ccccc3C2(C)C)cc1)C(F)(F)F]

The title compounds were prepared in an analogous manner to Intermediate 11 from Intermediate 8 (0.35 g, 0.82 mmol), 1,1-dimethyl-2,3-dihydro-1H-inden-2-amine hydrochloride (0.17 g, 0.86 mmol, CAS 74413-86-8), Cs₂CO₃ (0.66 g, 2.05 mmol), tBuBrettphosPdG3 (35 mg, 0.04 mmol) and RuPhos (38 mg, 0.08 mmol) in 1,4-dioxane (5 mL) at 120° C. for 2 h. Purified by preparative HPLC (X-Select CSH C18, 19×250 mm×5 μm, flow rate: 17 mL/min, 5 mM aqueous NH₄HCO₃ with MeCN 10% to 50% over 0.1 min, to 78% over 18.9 min, ramped to 98% over 0.1 min and held for 2 min). Chiral purification was carried out by preparative SFC (Chiralcel OJ-H, 30×250 mm×5 μm, temp 30° C., Back Pressure 100 bar, flow rate: 100 g/min, 70% CO₂ with 30% MeOH modifier) to provide Example 14 (75 mg) as Peak 1 and Example 15 (52 mg) as Peak 2. Example 14: ¹H NMR (400 MHz; DMSO-d₆) δ: 7.21-7.12 (m, 6H), 6.79 (d, 2H), 5.93 (d, 1H), 5.83 (dd, 1H), 3.99 (dd, 1H), 3.62-3.56 (m, 4H), 3.29-3.11 (m, 5H), 2.76-2.70 (m, 4H), 1.32 (s, 3H), 1.09 (s, 3H). LCMS (Method A): 510.3 [M+H]⁺. Example 15: ¹H NMR (400 MHz; DMSO-d₆) δ: 7.21-7.12 (m, 6H), 6.79 (d, 2H), 5.93 (d, 1H), 5.84 (dd, 1H), 4.00 (dd, 1H), 3.62-3.56 (m, 4H), 3.29-3.11 (m, 5H), 2.76-2.70 (m, 4H), 1.31 (s, 3H), 1.09 (s, 3H). LCMS (Method A): 510.3 [M+H]⁺.

### Example 16: N-((1S)-1-(4-((1,1-Dimethyl-2,3-dihydro-1H-inden-2-yl)amino)phenyl)-2,2,2-trifluoroethyl)-4-hydroxy-N,4-dimethylpiperidine-1-carboxamide—Isomer 1

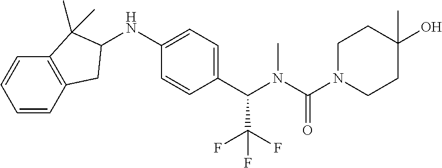 [SMILES: CN(C(=O)N1CCC(C)(O)CC1)[C@@H](c1ccc(NC2Cc3ccccc3C2(C)C)cc1)C(F)(F)F]

### Example 17: N-((1S)-1-(4-((1,1-Dimethyl-2,3-dihydro-1H-inden-2-yl)amino)phenyl)-2,2,2-trifluoroethyl)-4-hydroxy-N,4-dimethylpiperidine-1-carboxamide—Isomer 2

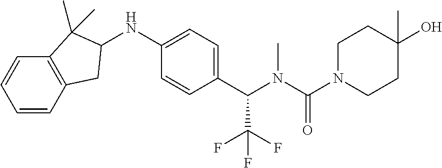 [SMILES: CN(C(=O)N1CCC(C)(O)CC1)[C@@H](c1ccc(NC2Cc3ccccc3C2(C)C)cc1)C(F)(F)F]

To a stirred solution of Intermediate 11 (2.5 g, 9.28 mmol) in THF (10 mL) at 0° C. was added TBAF (1M in THF, 1.35 mL, 1.35 mmol) and the mixture was stirred at RT for 3 h. The mixture was quenched with ice cold water and then extracted with EtOAc. The organic layer was concentrated under reduced pressure. The crude product was purified by preparative HPLC (Luna C18, 21.1×250 mm×5 μm, flow rate: 18 mL/min, 10 mM aqueous NH₄HCO₃ with MeCN 30% to 95% over 4.15 min, held at 95% for 12.85 min). Chiral purification was carried out by preparative SFC (Chiralcel OJ-H, 30×150 mm×5 μm, temp 30° C., Back Pressure 100 bar, flow rate: 100 g/min, 80% CO₂ with 20% MeOH modifier) to provide Example 16 (14 mg) as Peak 1 and Example 17 (17 mg) as Peak 2. Example 16: ¹H NMR (400 MHz; DMSO-d₆) δ: 7.21-7.12 (m, 6H), 6.79 (d, 2H), 5.92-5.80 (m, 2H), 4.33 (s, 1H), 3.99 (dd, 1H), 3.22-3.05 (m, 5H), 2.73 (dd, 1H), 2.64 (s, 3H), 1.57-1.35 (m, 4H), 1.32 (s, 3H), 1.12 (s, 3H), 1.09 (s, 3H). LCMS (Method A): 490.6 [M+H]⁺. Example 17: ¹H NMR (400 MHz; DMSO-d₆) δ: 7.21-7.12 (m, 6H), 6.78 (d, 2H), 5.92-5.80 (m, 2H), 4.33 (s, 1H), 3.99 (dd, 1H), 3.22-3.05 (m, 5H), 2.73 (dd, 1H), 2.64 (s, 3H), 1.57-1.35 (m, 4H), 1.32 (s, 3H), 1.12 (s, 3H), 1.09 (s, 3H). LCMS (Method A): 490.7 [M+H]⁺.

### Example 18: N-((1S)-1-(4-((1,1-Dimethyl-2,3-dihydro-1H-inden-2-yl)amino)phenyl)-2,2,2-trifluoroethyl)-N-methyltetrahydro-2H-pyran-4-carboxamide—Isomer 1

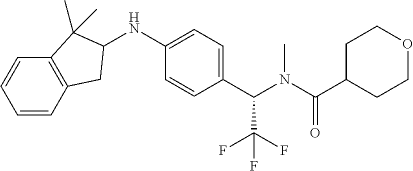 [SMILES: CN(C(=O)C1CCOCC1)[C@@H](c1ccc(NC2Cc3ccccc3C2(C)C)cc1)C(F)(F)F]

### Example 19: N-((1S)-1-(4-((1,1-Dimethyl-2,3-dihydro-1H-inden-2-yl)amino)phenyl)-2,2,2-trifluoroethyl)-N-methyltetrahydro-2H-pyran-4-carboxamide—Isomer 2

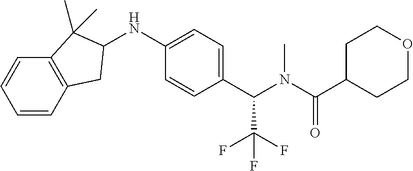 [SMILES: CN(C(=O)C1CCOCC1)[C@@H](c1ccc(NC2Cc3ccccc3C2(C)C)cc1)C(F)(F)F]

The title compounds were prepared in an analogous manner to Intermediate 11 from Intermediate 12 (0.18 g, 0.47 mmol), 1,1-dimethyl-2,3-dihydro-1H-inden-2-amine hydrochloride (0.11 g, 0.57 mmol, CAS 74413-86-8), Cs₂CO₃ (0.61 g, 1.88 mmol), tBuBrettphosPdG3 (40 mg, 0.05 mmol) and RuPhos (44 mg, 0.09 mmol) in 1,4-dioxane (2 mL) at 120° C. for 2 h. Purified by preparative HPLC (XBridge C18, 19×250 mm×5 μm, flow rate: 18 mL/min, 5 mM aqueous NH₄HCO₃ with MeCN 45% to 76% over 23 min, ramped to 98% over 0.1 min and held for 2 min). Chiral purification was carried out by preparative SFC (Chiralpak® IG, 30×250 mm×5 μm, temp 30° C., Back Pressure 100 bar, flow rate: 100 g/min, 60% CO₂ with 40% MeOH modifier) to provide Example 18 (12 mg) as Peak 1 and Example 19 (11 mg) as Peak 2. Example 18: ¹H NMR (400 MHz; DMSO-d₆) δ: 7.22-7.11 (m, 4H), 7.04 (d, 2H), 6.79 (d, 2H), 6.38 (dd, 1H), 5.94 (d, 1H), 3.99 (dd, 1H), 3.92-3.84 (m, 2H), 3.44-3.38 (m, 2H), 3.17 (dd, 1H), 3.02-2.92 (m, 1H), 2.89 (s, 3H), 2.73 (dd, 1H), 1.67-1.55 (m, 4H), 1.32 (s, 3H), 1.09 (s, 3H). LCMS (Method A): 461.3 [M+H]⁺. Example 19: ¹H NMR (400 MHz; DMSO-d₆) δ: 7.22-7.11 (m, 4H), 7.04 (d, 2H), 6.79 (d, 2H), 6.37 (dd, 1H), 5.94 (d, 1H), 3.99 (dd, 1H), 3.92-3.84 (m, 2H), 3.44-3.38 (m, 2H), 3.17 (dd, 1H), 3.02-2.92 (m, 1H), 2.89 (s, 3H), 2.73 (dd, 1H), 1.67-1.55 (m, 4H), 1.32 (s, 3H), 1.09 (s, 3H). LCMS (Method A): 461.3 [M+H]⁺.

### Example 20: N-((1S)-1-(4-((1,1-Dimethyl-2,3-dihydro-1H-inden-2-yl)amino)phenyl)-2,2,2-trifluoroethyl)-N-methyl-2-oxo-1,2-dihydropyridine-4-carboxamide—Isomer 1

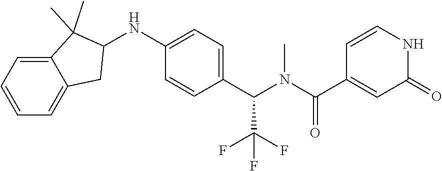 [SMILES: CN(C(=O)c1cc[nH]c(=O)c1)[C@@H](c1ccc(NC2Cc3ccccc3C2(C)C)cc1)C(F)(F)F]

### Example 21: N-((1S)-1-(4-((1,1-Dimethyl-2,3-dihydro-1H-inden-2-yl)amino)phenyl)-2,2,2-trifluoroethyl)-N-methyl-2-oxo-1,2-dihydropyridine-4-carboxamide—Isomer 2

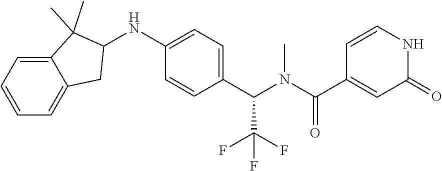 [SMILES: CN(C(=O)c1cc[nH]c(=O)c1)[C@@H](c1ccc(NC2Cc3ccccc3C2(C)C)cc1)C(F)(F)F]

The title compounds were prepared in an analogous manner to Intermediate 4 from Intermediate 14 (0.11 g, 0.31 mmol), 2-oxo-1,2-dihydropyridine-4-carboxylic acid (0.05 g, 0.37 mmol, CAS 22282-72-0), POCl₃ (0.03 mL, 0.31 mmol) and DMAP (2 mg) in pyridine (5 mL) at RT for 3 h. The crude product was purified by preparative HPLC (XBridge-Phenyl, 19×250 mm×5 μm, flow rate: 17 mL/min, 10 mM aqueous NH₄HCO₃ with MeCN 20% to 55% over 5 min, held at 55% for 6 min, ramped to 98% for 0.1 min and held at 98% for 3.9 min). Chiral purification was carried out by preparative SFC (Chiralpak® IG, 30×250 mm×5 μm, temp 30° C., Back Pressure 100 bar, flow rate: 110 g/min, 70% CO₂ with 30% 1:1 MeCN/MeOH modifier) to provide Example 20 (13 mg) as Peak 1 and Example 21 (14 mg) as Peak 2. Example 20: ¹H NMR (400 MHz; DMSO-d₆) δ: 11.84 (s, 1H), 7.54-7.45 (br m, 1H), 7.21-7.09 (m, 6H), 6.81 (d, 2H), 6.35 (dd, 1H), 6.26 (s, 1H), 6.14-5.98 (m, 2H), 4.01 (dd, 1H), 3.17 (dd, 1H), 2.82-2.72 (m, 4H), 1.32 (s, 3H), 1.09 (s, 3H). LCMS (Method A): 470.6 [M+H]⁺. Example 21: ¹H NMR (400 MHz; DMSO-d₆) δ: 11.85 (s, 1H), 7.54-7.45 (br m, 1H), 7.21-7.09 (m, 6H), 6.81 (d, 2H), 6.35 (dd, 1H), 6.26 (s, 1H), 6.14-5.984 (m, 2H), 4.01 (dd, 1H), 3.17 (dd, 1H), 2.82-2.72 (m, 4H), 1.32 (s, 3H), 1.09 (s, 3H). LCMS (Method A): 470.3 [M+H]⁺.

### Example 22: N-((1S)-1-(4-((1,1-Dimethyl-2,3-dihydro-1H-inden-2-yl)amino)phenyl)-2,2,2-trifluoroethyl)-4-hydroxy-N-methylpiperidine-1-carboxamide—Isomer 1

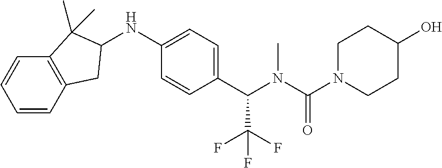 [SMILES: CN(C(=O)N1CCC(O)CC1)[C@@H](c1ccc(NC2Cc3ccccc3C2(C)C)cc1)C(F)(F)F]

### Example 23: N-((1S)-1-(4-((1,1-Dimethyl-2,3-dihydro-1H-inden-2-yl)amino)phenyl)-2,2,2-trifluoroethyl)-4-hydroxy-N-methylpiperidine-1-carboxamide—Isomer 2

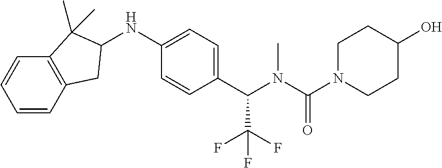 [SMILES: CN(C(=O)N1CCC(O)CC1)[C@@H](c1ccc(NC2Cc3ccccc3C2(C)C)cc1)C(F)(F)F]

The title compounds were prepared in an analogous manner to Intermediate 11 from Intermediate 16 (0.30 g, 0.64 mmol), 1,1-dimethyl-2,3-dihydro-1H-inden-2-amine hydrochloride (0.15 g, 0.77 mmol, CAS 74413-86-8), Cs₂CO₃ (0.84 g, 2.57 mmol), tBuBrettphosPdG3 (54 mg, 0.06 mmol) and RuPhos (59 mg, 0.13 mmol) in 1,4-dioxane (10 mL) at 120° C. for 2 h. Purified by preparative HPLC (XBridge-Phenyl, 19×250 mm×5 μm, flow rate: 17 mL/min, 10 mM aqueous NH₄HCO₃ with MeCN 30% to 65% over 8 min, held at 65% for 6.5 min, ramped to 98% over 0.1 min and held for 5.4 min). Chiral purification was carried out by preparative SFC (Chiralcel OG-H, 21×250 mm×5 μm, temp 30° C., Back Pressure 100 bar, flow rate: 100 g/min, 85% CO₂ with 15% MeOH modifier) to provide Example 22 (7.4 mg) as Peak 1 and Example 23 (7.6 mg) as Peak 2. Example 22: ¹H NMR (400 MHz; DMSO-d₆) δ: 7.22-7.12 (m, 6H), 6.79 (d, 2H), 5.93-5.81 (m, 2H), 4.70 (d, 1H), 3.99 (dd, 1H), 3.69-3.60 (m, 1H), 3.51-3.38 (m, 2H), 3.17 (dd, 1H), 2.97 (dt, 1H), 2.84 (dt, 1H), 2.73 (dd, 1H), 2.65 (s, 3H), 1.79-1.65 (m, 2H), 1.46-1.38 (m, 1H), 1.32 (s, 3H), 1.31-1.22 (m, 1H), 1.09 (s, 3H). LCMS (Method A): 476.3 [M+H]⁺. Example 23: ¹H NMR (400 MHz; DMSO-d₆) δ: 7.22-7.12 (m, 6H), 6.78 (d, 2H), 5.93-5.81 (m, 2H), 4.70 (d, 1H), 3.99 (dd, 1H), 3.69-3.60 (m, 1H), 3.51-3.38 (m, 2H), 3.17 (dd, 1H), 2.97 (dt, 1H), 2.84 (dt, 1H), 2.73 (dd, 1H), 2.65 (s, 3H), 1.79-1.65 (m, 2H), 1.46-1.38 (m, 1H), 1.32 (s, 3H), 1.31-1.22 (m, 1H), 1.09 (s, 3H). LCMS (Method A): 476.3 [M+H]⁺.

### Example 24: 3-Acetamido-N-((1S)-1-(4-((1,1-dimethyl-2,3-dihydro-1H-inden-2-yl)amino)phenyl)-2,2,2-trifluoroethyl)-N-methylpropanamide—Isomer 1

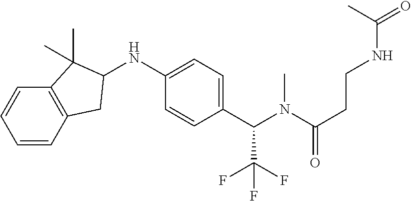 [SMILES: CC(=O)NCCC(=O)N(C)[C@@H](c1ccc(NC2Cc3ccccc3C2(C)C)cc1)C(F)(F)F]

### Example 25: 3-Acetamido-N-((1S)-1-(4-((1,1-dimethyl-2,3-dihydro-1H-inden-2-yl)amino)phenyl)-2,2,2-trifluoroethyl)-N-methylpropanamide—Isomer 2

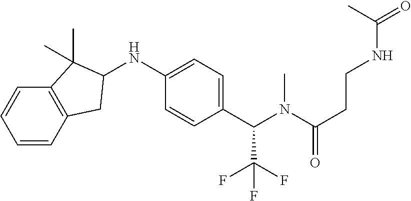 [SMILES: CC(=O)NCCC(=O)N(C)[C@@H](c1ccc(NC2Cc3ccccc3C2(C)C)cc1)C(F)(F)F]

The title compounds were prepared in an analogous manner to Intermediate 11 from Intermediate 17 (0.30 g, 0.79 mmol), 1,1-dimethyl-2,3-dihydro-1H-inden-2-amine hydrochloride (0.16 g, 0.79 mmol, CAS 74413-86-8), Cs₂CO₃ (1.02 g, 3.12 mmol), tBuBrettphosPdG3 (67 mg, 0.08 mmol) and RuPhos (79 mg, 0.16 mmol) in 1,4-dioxane (5 mL) at 120° C. for 2 h. Purified by preparative HPLC (XBridge-Phenyl, 19×250 mm×5 μm, flow rate: 17 mL/min, 10 mM aqueous NH₄HCO₃ with MeCN 35% to 65% over 17.5 min, ramped to 98% over 0.1 min and held for 2.9 min). Chiral purification was carried out by preparative SFC (Chiralpak® IG, 30×250 mm×5 μm, temp 30° C., Back Pressure 100 bar, flow rate: 110 g/min, 70% CO₂ with 30% 1:1 MeCN/MeOH modifier) to provide Example 24 (13 mg) as Peak 1 and Example 25 (10 mg) as Peak 2. Example 24: ¹H NMR (400 MHz; DMSO-d₆) δ: 7.89 (t, 1H), 7.22-7.12 (m, 4H), 7.06 (d, 2H), 6.78 (d, 2H), 6.34 (dd, 1H), 5.93 (d, 1H), 3.99 (dd, 1H), 3.28-3.25 (m, 2H), 3.17 (dd, 1H), 2.80 (s, 3H), 2.73 (dd, 1H), 2.68-2.54 (m, 2H), 1.78 (s, 3H), 1.31 (s, 3H), 1.08 (s, 3H). LCMS (Method A): 462.3 [M+H]⁺. Example 25: ¹H NMR (400 MHz; DMSO-d₆) δ: 7.89 (t, 1H), 7.22-7.12 (m, 4H), 7.06 (d, 2H), 6.78 (d, 2H), 6.34 (dd, 1H), 5.93 (d, 1H), 3.99 (dd, 1H), 3.28-3.25 (m, 2H), 3.17 (dd, 1H), 2.80 (s, 3H), 2.73 (dd, 1H), 2.68-2.54 (m, 2H), 1.78 (s, 3H), 1.31 (s, 3H), 1.08 (s, 3H). LCMS (Method A): 462.2 [M+H]⁺.

### Example 26: N-((1S)-1-(4-((1,1-Dimethyl-2,3-dihydro-1H-inden-2-yl)amino)phenyl)-2,2,2-trifluoroethyl)-N-methyloxazole-4-carboxamide—Isomer 1

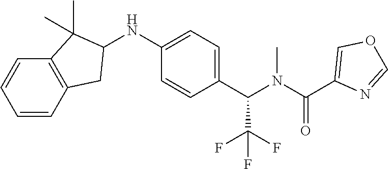 [SMILES: CN(C(=O)c1cocn1)[C@@H](c1ccc(NC2Cc3ccccc3C2(C)C)cc1)C(F)(F)F]

### Example 27: N-((1S)-1-(4-((1,1-Dimethyl-2,3-dihydro-1H-inden-2-yl)amino)phenyl)-2,2,2-trifluoroethyl)-N-methyloxazole-4-carboxamide—Isomer 2

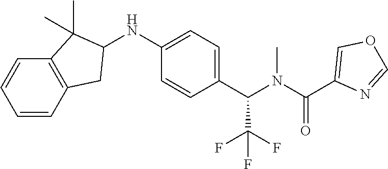 [SMILES: CN(C(=O)c1cocn1)[C@@H](c1ccc(NC2Cc3ccccc3C2(C)C)cc1)C(F)(F)F]

The title compounds were prepared in an analogous manner to Intermediate 11 from Intermediate 18 (0.35 g, 0.96 mmol), 1,1-dimethyl-2,3-dihydro-1H-inden-2-amine hydrochloride (0.19 g, 0.96 mmol, CAS 74413-86-8), Cs₂CO₃ (1.26 g, 3.85 mmol), tBuBrettphosPdG3 (82 mg, 0.10 mmol) and RuPhos (90 mg, 0.19 mmol) in 1,4-dioxane (5 mL) at 120° C. for 2 h. Purified by preparative HPLC (XBridge C18, 19×250 mm×5 μm, flow rate: 18 mL/min, 10 mM aqueous NH₄HCO₃ with MeCN 40% to 75% over 7 min, held at 75% for 7.5 min, ramped to 98% over 0.1 min and held for 7.4 min). Chiral purification was carried out by preparative SFC (Chiralpak® IG, 30×250 mm×5 μm, temp 30° C., Back Pressure 100 bar, flow rate: 110 g/min, 70% CO₂ with 30% 1:1 MeCN/MeOH modifier) to provide Example 26 (35 mg) as Peak 1 and Example 27 (53 mg) as Peak 2. Example 26: ¹H NMR (400 MHz; DMSO-d₆) δ: 8.79-8.52 (br m, 2H), 7.22-7.12 (m, 6H), 6.81 (d, 2H), 6.47 (br s, 1H), 6.00 (d, 1H), 4.00 (dd, 1H), 3.17 (dd, 1H), 3.08 (br s, 2H), 2.80 (br s, 1H), 2.74 (dd, 1H), 1.32 (s, 3H), 1.09 (s, 3H). LCMS (Method A): 444.2 [M+H]⁺. Example 27: ¹H NMR (400 MHz; DMSO-d₆) δ: 8.79-8.52 (br m, 2H), 7.22-7.12 (m, 6H), 6.81 (d, 2H), 6.47 (br s, 1H), 6.00 (d, 1H), 4.01 (dd, 1H), 3.17 (dd, 1H), 3.08 (br s, 2H), 2.80 (br s, 1H), 2.74 (dd, 1H), 1.32 (s, 3H), 1.09 (s, 3H). LCMS (Method A): 444.2 [M+H]⁺.

### Example 28: N-((1S)-1-(4-((1,1-Dimethyl-2,3-dihydro-1H-inden-2-yl)amino)phenyl)-2,2,2-trifluoroethyl)-2-methoxy-N-methylacetamide—Isomer 1

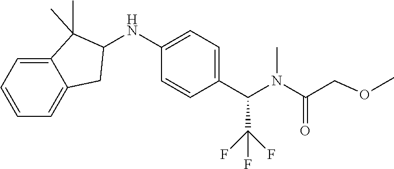 [SMILES: COCC(=O)N(C)[C@@H](c1ccc(NC2Cc3ccccc3C2(C)C)cc1)C(F)(F)F]

### Example 29: N-((1S)-1-(4-((1,1-Dimethyl-2,3-dihydro-1H-inden-2-yl)amino)phenyl)-2,2,2-trifluoroethyl)-2-methoxy-N-methylacetamide—Isomer 2

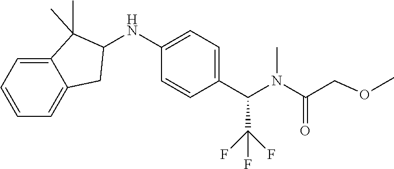 [SMILES: COCC(=O)N(C)[C@@H](c1ccc(NC2Cc3ccccc3C2(C)C)cc1)C(F)(F)F]

The title compounds were prepared in an analogous manner to Intermediate 11 from Intermediate 20 (0.38 g, 1.12 mmol), 1,1-dimethyl-2,3-dihydro-1H-inden-2-amine hydrochloride (0.22 g, 1.12 mmol, CAS 74413-86-8), Cs₂CO₃ (1.46 g, 4.48 mmol), tBuBrettphosPdG3 (0.14 g, 0.17 mmol) and RuPhos (0.16 g, 0.34 mmol) in 1,4-dioxane (5 mL) at 120° C. for 1 h. Purified by preparative HPLC (XBridge C18, 19×250 mm×5 μm, flow rate: 17 mL/min, 10 mM aqueous NH₄HCO₃ with MeCN 40% to 70% over 9 min, held at 75% for 7.5 min, ramped to 98% over 0.1 min and held for 4.4 min). Chiral purification was carried out by preparative SFC (Chiralpak® AD-H, 30×250 mm×5 μm, temp 30° C., Back Pressure 100 bar, flow rate: 100 g/min, 80% CO₂ with 20% MeOH modifier) to provide Example 28 (9.6 mg) as Peak 1 and Example 29 (9.4 mg) as Peak 2. Example 28: ¹H NMR (400 MHz; DMSO-d₆) δ: 7.22-7.12 (m, 4H), 7.06 (d, 2H), 6.79 (d, 2H), 6.30 (dd, 1H), 5.95 (d, 1H), 4.22 (dd, 2H), 3.99 (dd, 1H), 3.17 (dd, 1H), 2.77 (s, 3H), 2.73 (dd, 1H), 1.31 (s, 3H), 1.09 (s, 3H)—3H obscured by solvent peak at 3.33. LCMS (Method A): 421.4 [M+H]⁺. Example 29: ¹H NMR (400 MHz; DMSO-d₆) δ: 7.22-7.12 (m, 4H), 7.06 (d, 2H), 6.79 (d, 2H), 6.30 (dd, 1H), 5.95 (d, 1H), 4.22 (dd, 2H), 3.99 (dd, 1H), 3.17 (dd, 1H), 2.77 (s, 3H), 2.73 (dd, 1H), 1.31 (s, 3H), 1.09 (s, 3H)—3H obscured by solvent peak at 3.33. LCMS (Method A): 421.4 [M+H]⁺.

### Example 30: N-((1S)-1-(4-((1,1-Dimethyl-2,3-dihydro-1H-inden-2-yl)amino)phenyl)-2,2,2-trifluoroethyl)-2-hydroxy-N-methylacetamide—Isomer 1

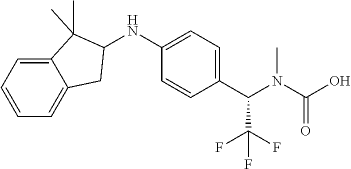 [SMILES: CN(C(=O)O)[C@@H](c1ccc(NC2Cc3ccccc3C2(C)C)cc1)C(F)(F)F]

### Example 31: N-((1S)-1-(4-((1,1-Dimethyl-2,3-dihydro-1H-inden-2-yl)amino)phenyl)-2,2,2-trifluoroethyl)-2-hydroxy-N-methylacetamide—Isomer 2

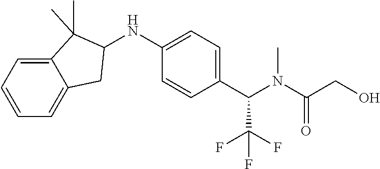 [SMILES: CN(C(=O)CO)[C@@H](c1ccc(NC2Cc3ccccc3C2(C)C)cc1)C(F)(F)F]

The title compounds were prepared from a 1:1 mixture of Example 28/29 (95 mg, 0.23 mmol) in DCM (10 mL) at 0° C. treated with boron tribromide (1M in DCM, 10 mL, 10 mmol) and stirred at 0° C. for 3 h. The mixture was concentrated under reduced pressure then triturated in diethyl ether. Purification was carried out by preparative SFC (Chiralpak® IG, 30×250 mm×5 μm, temp 30° C., Back Pressure 100 bar, flow rate: 50 g/min, 70% CO₂ with 30% MeOH modifier) to provide Example 30 (11 mg) as Peak 1 and Example 31 (10 mg) as Peak 2. Example 30: ¹H NMR (400 MHz; DMSO-d₆) δ: 7.22-7.12 (m, 4H), 7.07 (d, 2H), 6.79 (d, 2H), 6.30 (dd, 1H), 5.95 (d, 1H), 4.82 (dd, 1H), 4.19 (d, 2H), 3.99 (dd, 1H), 3.17 (dd, 1H), 2.76 (s, 3H), 2.72-2.66 (m, 1H), 1.31 (s, 3H), 1.09 (s, 3H). LCMS (Method A): 407.2 [M+H]⁺. Example 31: ¹H NMR (400 MHz; DMSO-d₆) δ: 7.22-7.12 (m, 4H), 7.07 (d, 2H), 6.79 (d, 2H), 6.30 (dd, 1H), 5.95 (d, 1H), 4.82 (dd, 1H), 4.19 (d, 2H), 3.99 (dd, 1H), 3.17 (dd, 1H), 2.76 (s, 3H), 2.72-2.66 (m, 1H), 1.32 (s, 3H), 1.08 (s, 3H). LCMS (Method A): 407.2 [M+H]⁺.

### Example 32: N-((1S)-1-(4-((1,1-Dimethyl-2,3-dihydro-1H-inden-2-yl)amino)phenyl)-2,2,2-trifluoroethyl)-3-methoxy-N-methylpropanamide—Isomer 1

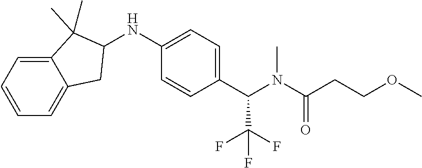 [SMILES: COCCC(=O)N(C)[C@@H](c1ccc(NC2Cc3ccccc3C2(C)C)cc1)C(F)(F)F]

### Example 33: N-((1S)-1-(4-((1,1-Dimethyl-2,3-dihydro-1H-inden-2-yl)amino)phenyl)-2,2,2-trifluoroethyl)-3-methoxy-N-methylpropanamide—Isomer 2

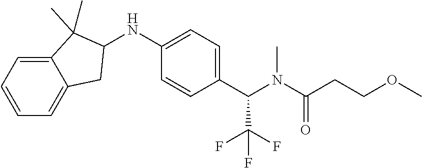 [SMILES: COCCC(=O)N(C)[C@@H](c1ccc(NC2Cc3ccccc3C2(C)C)cc1)C(F)(F)F]

The title compounds were prepared from Intermediate 22 (0.07 g, 0.16 mmol), purified by chiral preparative SFC (Chiralcel-OJ-H, 21×250 mm×5 μm, temp 30° C., Back Pressure 100 bar, flow rate: 60 g/min, 92% CO₂ with 8% MeOH modifier) to provide Example 32 (16 mg) as Peak 1 and Example 33 (18 mg) as Peak 2. Example 32: ¹H NMR (400 MHz; DMSO-d₆) δ: 7.22-7.12 (m, 4H), 7.05 (d, 2H), 6.78 (d, 2H), 6.34 (dd, 1H), 5.93 (d, 1H), 3.99 (dd, 1H), 3.60 (t, 2H), 3.24 (s, 3H), 3.17 (dd, 1H), 2.83 (s, 3H), 2.78-2.65 (m, 3H), 1.32 (s, 3H), 1.08 (s, 3H). LCMS (Method A): 435.5 [M+H]⁺. Example 33: ¹H NMR (400 MHz; DMSO-d₆) δ: 7.22-7.12 (m, 4H), 7.05 (d, 2H), 6.78 (d, 2H), 6.34 (dd, 1H), 5.93 (d, 1H), 3.99 (dd, 1H), 3.60 (t, 2H), 3.24 (s, 3H), 3.17 (dd, 1H), 2.83 (s, 3H), 2.78-2.65 (m, 3H), 1.32 (s, 3H), 1.09 (s, 3H). LCMS (Method A): 435.5 [M+H]⁺.

### Example 34: N-((1S)-1-(4-((1,1-Dimethyl-2,3-dihydro-1H-inden-2-yl)amino)phenyl)-2,2,2-trifluoroethyl)-3-hydroxy-N-methylpropanamide—Isomer 1

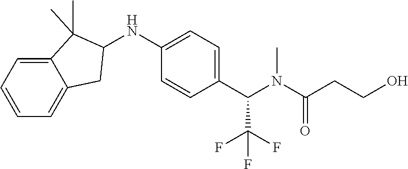 [SMILES: CN(C(=O)CCO)[C@@H](c1ccc(NC2Cc3ccccc3C2(C)C)cc1)C(F)(F)F]

### Example 35: N-((1S)-1-(4-((1,1-Dimethyl-2,3-dihydro-1H-inden-2-yl)amino)phenyl)-2,2,2-trifluoroethyl)-3-hydroxy-N-methylpropanamide—Isomer 2

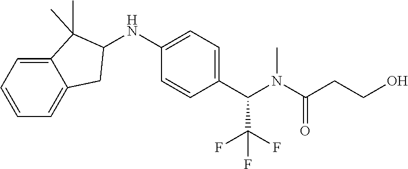 [SMILES: CN(C(=O)CCO)[C@@H](c1ccc(NC2Cc3ccccc3C2(C)C)cc1)C(F)(F)F]

The title compounds were prepared from Intermediate 22 (0.20 g, 0.46 mmol) in DCM (10 mL) at −40° C. treated with boron tribromide (0.34 g, 1.38 mmol) and stirred at −40° C. for 1 h. The mixture was diluted with water and extracted with DCM. The organic layer was dried over Na₂SO₄, filtered and concentrated under reduced pressure. Purified by preparative HPLC (XBridge-phenyl, 19×250 mm×5 μm, flow rate: 17 mL/min, 10 mM aqueous NH₄HCO₃ with MeCN 50% to 64% over 20 min, ramped to 98% over 0.1 min). Chiral purification was carried out by preparative SFC (Chiralpak®-IG-3, 30×250 mm×5 μm, temp 30° C., Back Pressure 100 bar, flow rate: 100 g/min, 70% CO₂ with 30% MeOH modifier) to provide Example 34 (28 mg) as Peak 1 and Example 35 (22 mg) as Peak 2. Example 34: ¹H NMR (400 MHz; DMSO-d₆) δ: 7.22-7.12 (m, 4H), 7.05 (d, 2H), 6.78 (d, 2H), 6.36 (dd, 1H), 5.92 (d, 1H), 4.55 (dd, 1H), 3.98 (dd, 1H), 3.70-3.66 (m, 2H), 3.16 (dd, 1H), 2.83 (s, 3H), 2.76-2.66 (m, 3H), 1.31 (s, 3H), 1.09 (s, 3H). LCMS (Method A): 421.2 [M+H]⁺. Example 35: ¹H NMR (400 MHz; DMSO-d₆) δ: 7.22-7.12 (m, 4H), 7.05 (d, 2H), 6.78 (d, 2H), 6.36 (dd, 1H), 5.92 (d, 1H), 4.55 (dd, 1H), 3.99 (dd, 1H), 3.70-3.66 (m, 2H), 3.16 (dd, 1H), 2.83 (s, 3H), 2.76-2.66 (m, 3H), 1.31 (s, 3H), 1.09 (s, 3H). LCMS (Method A): 421.2 [M+H]⁺.

### Example 36: N-((1S)-1-(6-((1,1-Dimethyl-2,3-dihydro-1H-inden-2-yl)amino)pyridin-3-yl)-2,2,2-trifluoroethyl)-N-methyltetrahydro-2H-thiopyran-4-carboxamide 1,1-dioxide—Isomer 1

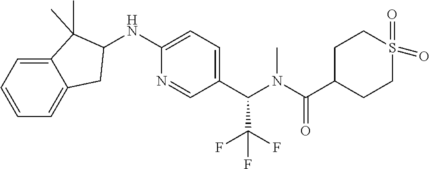 [SMILES: CN(C(=O)C1CCS(=O)(=O)CC1)[C@@H](c1ccc(NC2Cc3ccccc3C2(C)C)nc1)C(F)(F)F]

### Example 37: N-((1S)-1-(6-((1,1-Dimethyl-2,3-dihydro-1H-inden-2-yl)amino)pyridin-3-yl)-2,2,2-trifluoroethyl)-N-methyltetrahydro-2H-thiopyran-4-carboxamide 1,1-dioxide—Isomer 2

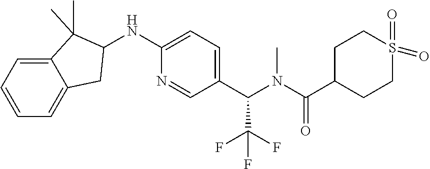 [SMILES: CN(C(=O)C1CCS(=O)(=O)CC1)[C@@H](c1ccc(NC2Cc3ccccc3C2(C)C)nc1)C(F)(F)F]

The title compounds were prepared in an analogous manner to Intermediate 3 from Intermediate 27 (0.40 g, 1.15 mmol), pyridine (4 mL), T3P® (50% EtOAc, 3.63 g, 5.72 mmol) and tetrahydro-2H-thiopyran-4-carboxylic acid 1,1-dioxide (0.51 g, 2.86 mmol, CAS 64096-87-3) at RT for 16 h. Purified by column chromatography (silica gel, eluting 40-45% EtOAc in petroleum ether). Chiral purification was carried out by preparative SFC (Chiralpak®-AD, 30×250 mm×5 μm, temp 30° C., Back Pressure 100 bar, flow rate: 100 g/min, 60% CO₂ with 40% MeOH/MeCN 1:1 modifier) to provide Example 36 (72 mg) as Peak 1 and Example 37 (69 mg) as Peak 2. Example 36: ¹H NMR (400 MHz; DMSO-d₆) δ: 8.04-7.94 (m, 1H), 7.50-7.40 (m, 1H), 7.22-7.12 (m, 4H), 6.99-6.90 (m, 1H), 6.78 (br d, 1H), 6.37 (dd, 1H), 4.56 (dd, 1H), 3.30-3.05 (m, 6H), 2.94 (s, 3H), 2.78 (dd, 1H), 2.12-1.94 (m, 4H), 1.28 (s, 3H), 1.09 (s, 3H). LCMS (Method A): 510.5 [M+H]⁺. Example 37: ¹H NMR (400 MHz; DMSO-d₆) δ: 8.04-7.94 (m, 1H), 7.50-7.40 (m, 1H), 7.22-7.12 (m, 4H), 7.01-6.90 (m, 1H), 6.69 (d, 1H), 6.37 (dd, 1H), 4.57 (dd, 1H), 3.30-3.05 (m, 6H), 2.94 (s, 3H), 2.78 (dd, 1H), 2.12-1.94 (m, 4H), 1.29 (s, 3H), 1.08 (s, 3H). LCMS (Method A): 510.5 [M+H]⁺.

### Example 38: N-((1S)-1-(4-((1,3-Dimethyl-2,3-dihydro-1H-inden-2-yl)amino)phenyl)-2,2,2-trifluoroethyl)-N-methyltetrahydro-2H-thiopyran-4-carboxamide 1,1-dioxide

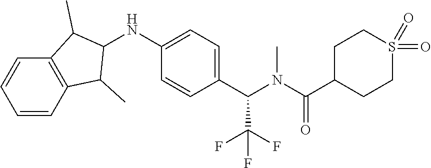 [SMILES: CC1c2ccccc2C(C)C1Nc1ccc([C@H](N(C)C(=O)C2CCS(=O)(=O)CC2)C(F)(F)F)cc1]

The title compound (18 mg) was prepared in an analogous manner to Intermediate 11 from Intermediate 3 (0.50 g, 1.16 mmol), 1,3-dimethyl-2,3-dihydro-1H-inden-2-amine (0.23 g, 1.16 mmol, CAS 61957-34-4), Cs₂CO₃ (1.13 g, 3.50 mmol), XPhosPdG3 (98 mg, 0.12 mmol) and RuPhos (0.11 g, 0.23 mmol) in 1,4-dioxane/DMF (4:1, 10 mL) at 130° C. for 1 h. Purified by preparative HPLC (XBridge C18, 19×250 mm×5 μm, flow rate: 18 mL/min, 10 mM aqueous NH₄HCO₃ with MeCN 30% to 60% over 5 min, held at 60% for 11 min, ramped to 100% over 0.1 min and held for 4.9 min). Chiral purification was carried out by preparative SFC (Chiralpak®-AD-3, 30×250 mm×5 μm, temp 30° C., Back Pressure 100 bar, flow rate: 100 g/min, 70% CO₂ with 30% MeOH modifier): ¹H NMR (400 MHz; DMSO-d₆) δ: 7.34-7.28 (m, 4H), 7.12-7.00 (m, 2H), 6.79 (d, 2H), 6.33 (dd, 1H), 5.60 (d, 1H), 4.40-4.32 (m, 1H), 3.29-3.06 (m, 7H), 2.88 (s, 3H), 2.11-1.95 (m, 4H), 1.20 (d, 6H). LCMS (Method C): 509.3 [M+H]⁺.

### Example 39: 2-Acetamido-N-((1S)-1-(4-((1,1-dimethyl-2,3-dihydro-1H-inden-2-yl)amino)phenyl)-2,2,2-trifluoroethyl)-N-methylacetamide—Isomer 1

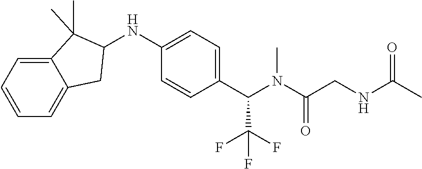 [SMILES: CC(=O)NCC(=O)N(C)[C@@H](c1ccc(NC2Cc3ccccc3C2(C)C)cc1)C(F)(F)F]

### Example 40: 2-Acetamido-N-((1S)-1-(4-((1,1-dimethyl-2,3-dihydro-1H-inden-2-ylamino)phenyl)-2,2,2-trifluoroethyl)-N-methylacetamide—Isomer 2

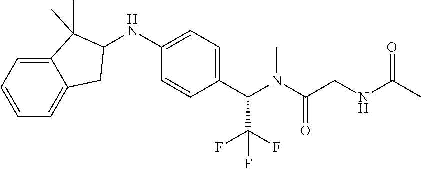 [SMILES: CC(=O)NCC(=O)N(C)[C@@H](c1ccc(NC2Cc3ccccc3C2(C)C)cc1)C(F)(F)F]

The title compounds were prepared in an analogous manner to Intermediate 3 from Intermediate 14 (24 mg, 0.21 mmol), pyridine (0.04 g, 0.52 mmol), T3P® (50% EtOAc, 0.55 g, 1.72 mmol) and N-acetylglycine (66 mg, 0.17 mmol, CAS 543-24-8) at RT for 16 h. Purified by column chromatography (silica gel, eluting 40-50% EtOAc in petroleum ether). Further purified by preparative HPLC (XBridge C18, 19×250 mm×5 μm, flow rate: 17 mL/min, 10 mM aqueous NH₄HCO₃ with MeCN 50% to 69% over 15 min, ramped to 98% over 0.1 min and held for 2 min). Chiral purification was carried out by preparative SFC (Chiralpak®-IG, 30×250 mm×5 μm, temp 30° C., Back Pressure 100 bar, flow rate: 100 g/min, 70% CO₂ with 30% MeOH modifier) to provide Example 39 (9 mg) as Peak 1 and Example 40 (9 mg) as Peak 2. Example 39: ¹H NMR (400 MHz; DMSO-d₆) δ: 8.19-8.07 (m, 1H), 7.22-7.12 (m, 4H), 7.06 (d, 2H), 6.79 (d, 2H), 6.28 (dd, 1H), 5.95 (d, 1H), 4.11-3.96 (m, 3H), 3.17 (dd, 1H), 2.83 (s, 3H), 2.73 (dd, 1H), 1.88 (s, 3H), 1.32 (s, 3H), 1.09 (s, 3H). LCMS (Method A): 470.3 [M+Na]⁺. Example 40: ¹H NMR (400 MHz; DMSO-d₆) δ: 8.19-8.07 (m, 1H), 7.22-7.12 (m, 4H), 7.06 (d, 2H), 6.79 (d, 2H), 6.28 (dd, 1H), 5.95 (d, 1H), 4.11-3.96 (m, 3H), 3.17 (dd, 1H), 2.83 (s, 3H), 2.73 (dd, 1H), 1.88 (s, 3H), 1.32 (s, 3H), 1.09 (s, 3H). LCMS (Method A): 470.3 [M+Na]⁺.

### Example 41: 1-Acetyl-N-((1S)-1-(4-((1,1-dimethyl-2,3-dihydro-1H-inden-2-yl)amino)phenyl)-2,2,2-trifluoroethyl)-N,3-dimethylazetidine-3-carboxamide—Isomer 1

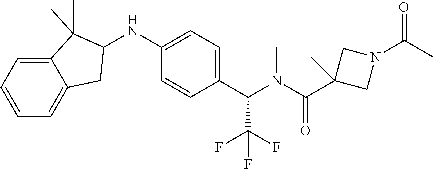 [SMILES: CC(=O)N1CC(C)(C(=O)N(C)[C@@H](c2ccc(NC3Cc4ccccc4C3(C)C)cc2)C(F)(F)F)C1]

### Example 42: 1-Acetyl-N-((1S)-1-(4-((1,1-dimethyl-2,3-dihydro-1H-inden-2-yl)amino)phenyl)-2,2,2-trifluoroethyl)-N,3-dimethylazetidine-3-carboxamide—Isomer 2

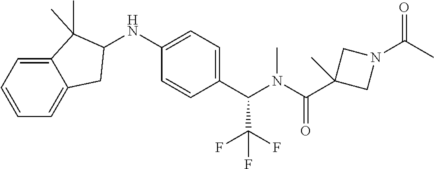 [SMILES: CC(=O)N1CC(C)(C(=O)N(C)[C@@H](c2ccc(NC3Cc4ccccc4C3(C)C)cc2)C(F)(F)F)C1]

The title compounds were prepared in an analogous manner to Intermediate 11 from Intermediate 30 (0.38 g, 0.93 mmol), 1,1-dimethyl-2,3-dihydro-1H-inden-2-amine hydrochloride (0.16 g, 1.02 mmol, CAS 74413-86-8), Cs₂CO₃ (0.90 g, 2.79 mmol), XPhosPdG3 (78 mg, 0.09 mmol) and RuPhos (86 mg, 0.18 mmol) in 1,4-dioxane (10 mL) at 120° C. for 1.5 h. Purified by preparative HPLC (XBridge C18, 19×250 mm×5 μm, flow rate: 17 mL/min, 10 mM aqueous NH₄HCO₃ with MeCN 25% to 70% over 7 min, held at 70% for 5.9 min, ramped to 98% over 0.1 min and held for 6 min). Chiral purification was carried out by preparative SFC (Chiralcel-OJ-H, 30×250 mm×5 μm, temp 30° C., Back Pressure 100 bar, flow rate: 100 g/min, 80% CO₂ with 20% MeOH modifier) to provide Example 41 (16 mg) as Peak 1 and Example 42 (18 mg) as Peak 2. Example 41: ¹H NMR (400 MHz; DMSO-d₆) δ: 7.22-7.13 (m, 4H), 7.10-7.05 (m, 2H), 6.79 (d, 2H), 6.33 (dd, 1H), 5.96 (d, 1H), 4.42 (dd, 1H), 4.15-3.95 (m, 2H), 3.90 (d, 1H), 3.66-3.63 (m, 1H), 3.17 (dd, 1H), 2.73 (dd, 1H), 2.66 (s, 3H), 1.76 (d, 3H), 1.51 (s, 3H), 1.32 (s, 3H), 1.08 (s, 3H). LCMS (Method A): 488.5 [M+H]⁺. Example 42: ¹H NMR (400 MHz; DMSO-d₆) δ: 7.22-7.13 (m, 4H), 7.10-7.05 (m, 2H), 6.79 (d, 2H), 6.33 (dd, 1H), 5.97 (d, 1H), 4.42 (dd, 1H), 4.15-3.95 (m, 2H), 3.90 (d, 1H), 3.66-3.63 (m, 1H), 3.17 (dd, 1H), 2.73 (dd, 1H), 2.66 (s, 3H), 1.76 (d, 3H), 1.51 (s, 3H), 1.32 (s, 3H), 1.09 (s, 3H). LCMS (Method A): 488.5 [M+H]⁺.

### Example 43: 1-Acetyl-N-((1S)-1-(4-((1,1-dimethyl-2,3-dihydro-1H-inden-2-yl)amino)phenyl)-2,2,2-trifluoroethyl)-3-fluoro-N-methylazetidine-3-carboxamide—Isomer 1

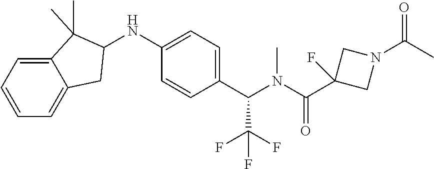 [SMILES: CC(=O)N1CC(F)(C(=O)N(C)[C@@H](c2ccc(NC3Cc4ccccc4C3(C)C)cc2)C(F)(F)F)C1]

### Example 44: 1-Acetyl-N-((1S)-1-(4-((1,1-dimethyl-2,3-dihydro-1H-inden-2-yl)amino)phenyl)-2,2,2-trifluoroethyl)-3-fluoro-N-methylazetidine-3-carboxamide—Isomer 2

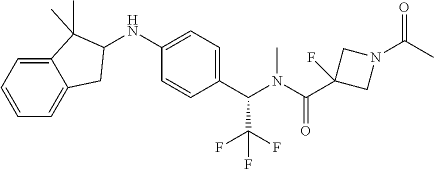 [SMILES: CC(=O)N1CC(F)(C(=O)N(C)[C@@H](c2ccc(NC3Cc4ccccc4C3(C)C)cc2)C(F)(F)F)C1]

The title compounds were prepared in an analogous manner to Intermediate 11 from Intermediate 33 (0.60 g, 1.46 mmol), 1,1-dimethyl-2,3-dihydro-1H-inden-2-amine hydrochloride (0.24 g, 1.19 mmol, CAS 74413-86-8), Cs₂CO₃ (1.42 g, 4.38 mmol), XPhosPdG3 (0.12 g, 0.15 mmol) and RuPhos (0.14 g, 0.29 mmol) in 1,4-dioxane/DMF (5:1, 12 mL) at 120° C. for 1 h. Purified by column chromatography (silica gel, eluting 50-60% EtOAc in hexane). Further purified by preparative HPLC (XBridge C18, 19×250 mm×5 μm, flow rate: 16 mL/min, 10 mM aqueous NH₄HCO₃ with MeCN 40% over 14.5 min, ramped to 98% over 0.1 min and held for 4.4 min). Chiral purification was carried out by preparative SFC (Chiralpak®-AD-H, 30×250 mm×5 μm, temp 30° C., Back Pressure 100 bar, flow rate: 110 g/min, 80% CO₂ with 20% MeOH modifier) to provide Example 43 (35 mg) as Peak 1 and Example 44 (41 mg) as Peak 2. Example 43: ¹H NMR (400 MHz; DMSO-d₆) δ: 7.22-7.08 (m, 6H), 6.80 (d, 2H), 6.25 (dd, 1H), 6.01 (d, 1H), 4.85-4.65 (m, 1H), 4.54-4.32 (m, 2H), 4.19-4.06 (m, 1H), 4.00 (dd, 1H), 3.18 (dd, 1H), 2.78 (s, 3H), 2.76 (dd, 1H), 1.82 (d, 3H), 1.32 (s, 3H), 1.09 (s, 3H). LCMS (Method A): 492.3 [M+H]⁺. Example 44: ¹H NMR (400 MHz; DMSO-d₆) δ: 7.22-7.08 (m, 6H), 6.80 (d, 2H), 6.25 (dd, 1H), 6.01 (d, 1H), 4.85-4.65 (m, 1H), 4.54-4.32 (m, 2H), 4.19-4.06 (m, 1H), 4.00 (dd, 1H), 3.18 (dd, 1H), 2.78 (s, 3H), 2.76 (dd, 1H), 1.82 (d, 3H), 1.32 (s, 3H), 1.09 (s, 3H). LCMS (Method A): 492.3 [M+H]⁺.

### Example 45: N-((1S)-1-(4-((1,1-Dimethyl-2,3-dihydro-1H-inden-2-yl)amino)phenyl)-2,2,2-trifluoroethyl)-N-methyl-2-(2-oxopyrrolidin-1-yl)acetamide—Isomer 1

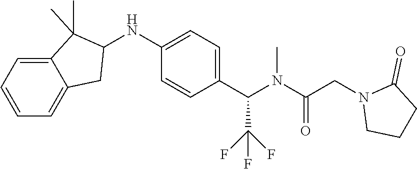 [SMILES: CN(C(=O)CN1CCCC1=O)[C@@H](c1ccc(NC2Cc3ccccc3C2(C)C)cc1)C(F)(F)F]

### Example 46: N-((1S)-1-(4-((1,1-Dimethyl-2,3-dihydro-1H-inden-2-yl)amino)phenyl)-2,2,2-trifluoroethyl)-N-methyl-2-(2-oxopyrrolidin-1-yl)acetamide—Isomer 2

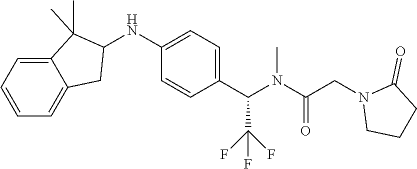 [SMILES: CN(C(=O)CN1CCCC1=O)[C@@H](c1ccc(NC2Cc3ccccc3C2(C)C)cc1)C(F)(F)F]

The title compounds were prepared in an analogous manner to Intermediate 11 from Intermediate 34 (0.40 g, 1.02 mmol), 1,1-dimethyl-2,3-dihydro-1H-inden-2-amine hydrochloride (0.13 g, 0.70 mmol, CAS 74413-86-8), Cs₂CO₃ (1.36 g, 4.07 mmol), XPhosPdG3 (86 mg, 0.10 mmol) and RuPhos (95 mg, 0.20 mmol) in 1,4-dioxane (4 mL) at 120° C. for 2 h. Purified by column chromatography (silica gel, eluting 20% EtOAc in petroleum ether). Further purified by preparative HPLC (XBridge C18, 19×250 mm×5 μm, flow rate: 17 mL/min, 10 mM aqueous NH₄HCO₃ with MeCN 30% to 60% over 6 min, held at 60% for 14 min, ramped to 98% over 0.1 min and held for 3.9 min). Chiral purification was carried out by preparative SFC (Chiralpak®-OD-H, 30×250 mm×5 μm, temp 30° C., Back Pressure 100 bar, flow rate: 110 g/min, 70% CO₂ with 30% MeOH modifier) to provide Example 45 (8 mg) as Peak 1 and Example 46 (9 mg) as Peak 2. Example 45: ¹H NMR (400 MHz; DMSO-d₆) δ: 7.22-7.12 (m, 4H), 7.06 (d, 2H), 6.79 (d, 2H), 6.25 (dd, 1H), 5.96 (d, 1H), 4.40-4.20 (m, 2H), 3.99 (dd, 1H), 3.40-3.35 (m, 2H), 3.17 (dd, 1H), 2.84 (s, 3H), 2.73 (dd, 1H), 2.26 (t, 2H), 2.01-1.92 (m, 2H), 1.31 (s, 3H), 1.09 (s, 3H). LCMS (Method A): 474.3 [M+H]⁺. Example 46: ¹H NMR (400 MHz; DMSO-d₆) δ: 7.22-7.12 (m, 4H), 7.06 (d, 2H), 6.79 (d, 2H), 6.25 (dd, 1H), 5.96 (d, 1H), 4.40-4.20 (m, 2H), 3.99 (dd, 1H), 3.40-3.35 (m, 2H), 3.17 (dd, 1H), 2.84 (s, 3H), 2.73 (dd, 1H), 2.26 (t, 2H), 2.01-1.92 (m, 2H), 1.32 (s, 3H), 1.09 (s, 3H). LCMS (Method A): 474.3 [M+H]⁺.

### Example 47: N-((1S)-1-(4-((1,1-Dimethyl-2,3-dihydro-1H-inden-2-yl)amino)phenyl)-2,2,2-trifluoroethyl)-N-methyl-3-(2-oxopyrrolidin-1-yl)propanamide—Isomer 1

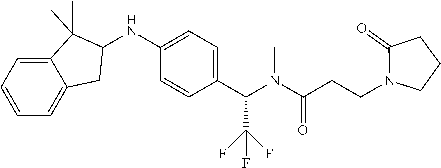 [SMILES: CN(C(=O)CCN1CCCC1=O)[C@@H](c1ccc(NC2Cc3ccccc3C2(C)C)cc1)C(F)(F)F]

### Example 48: N-((1S)-1-(4-((1,1-Dimethyl-2,3-dihydro-1H-inden-2-yl)amino)phenyl)-2,2,2-trifluoroethyl)-N-methyl-3-(2-oxopyrrolidin-1-yl)propanamide—Isomer 2

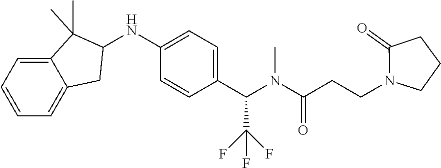 [SMILES: CN(C(=O)CCN1CCCC1=O)[C@@H](c1ccc(NC2Cc3ccccc3C2(C)C)cc1)C(F)(F)F]

The title compounds were prepared in an analogous manner to Intermediate 11 from Intermediate 35 (0.30 g, 0.86 mmol), 1,1-dimethyl-2,3-dihydro-1H-inden-2-amine hydrochloride (0.17 g, 0.86 mmol, CAS 74413-86-8), Cs₂CO₃ (0.84 g, 2.58 mmol), tBuBrettphosPdG3 (73 mg, 0.09 mmol) and RuPhos (80 mg, 0.17 mmol) in 1,4-dioxane (5 mL) at 120° C. for 2 h. Purified by preparative HPLC (XBridge C18, 19×250 mm×5 μm, flow rate: 17 mL/min, 10 mM aqueous NH₄HCO₃ with MeCN 25% to 65% over 34 min, held at 65% for 3 min, ramped to 95% over 0.1 min and held for 2 min). Chiral purification was carried out by preparative SFC (Chiralpak®-IG-3, 30×250 mm×5 μm, temp 30° C., Back Pressure 100 bar, flow rate: 100 g/min, 70% CO₂ with 30% MeOH modifier) to provide Example 47 (8 mg) as Peak 1 and Example 48 (9 mg) as Peak 2. Example 47: ¹H NMR (400 MHz; DMSO-d₆) δ: 7.22-7.12 (m, 4H), 7.06 (d, 2H), 6.78 (d, 2H), 6.34 (dd, 1H), 5.94 (d, 1H), 3.99 (dd, 1H), 3.43 (t, 2H), 3.32 (t, 2H), 3.17 (dd, 1H), 2.82 (s, 3H), 2.73 (dd, 1H), 2.65 (t, 2H), 2.18 (t, 2H), 1.92-1.83 (m, 2H), 1.31 (s, 3H), 1.09 (s, 3H). LCMS (Method A): 488.5 [M+H]⁺. Example 48: ¹H NMR (400 MHz; DMSO-d₆) δ: 7.22-7.12 (m, 4H), 7.06 (d, 2H), 6.78 (d, 2H), 6.34 (dd, 1H), 5.94 (d, 1H), 3.99 (dd, 1H), 3.43 (t, 2H), 3.32 (t, 2H), 3.17 (dd, 1H), 2.82 (s, 3H), 2.73 (dd, 1H), 2.65 (t, 2H), 2.18 (t, 2H), 1.92-1.83 (m, 2H), 1.32 (s, 3H), 1.09 (s, 3H). LCMS (Method A): 488.5 [M+H]⁺.

### Example 49: 5-(((1S)-1-(4-((1,1-Dimethyl-2,3-dihydro-1H-inden-2-yl)amino)phenyl)-2,2,2-trifluoroethyl)(methyl)amino)-5-oxopentanoic Acid—Isomer 1

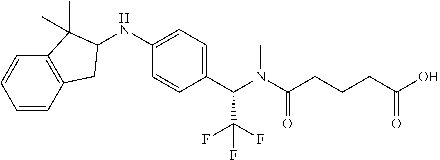 [SMILES: CN(C(=O)CCCC(=O)O)[C@@H](c1ccc(NC2Cc3ccccc3C2(C)C)cc1)C(F)(F)F]

### Example 50: 5-(((1S)-1-(4-((1,1-Dimethyl-2,3-dihydro-1H-inden-2-yl)amino)phenyl)-2,2,2-trifluoroethyl)(methyl)amino)-5-oxopentanoic Acid—Isomer 2

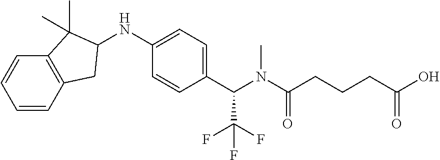 [SMILES: CN(C(=O)CCCC(=O)O)[C@@H](c1ccc(NC2Cc3ccccc3C2(C)C)cc1)C(F)(F)F]

The title compounds were prepared from Intermediate 37 (0.24 g, 0.49 mmol) in THF/water (4:1 20 mL) treated with lithium hydroxide monohydrate (0.12 g, 2.93 mmol) and stirred at RT for 16 h. The mixture was diluted with water and neutralized with citric acid. The solid was filtered and dried under reduced pressure. Purified by preparative HPLC (XBridge C18, 19×250 mm×5 μm, flow rate: 17 mL/min, 10 mM aqueous NH₄HCO₃ with MeCN 10% to 50% over 6 min, held at 50% for 8.5 min ramped to 98% over 0.1 min, held for 2.4 min). Chiral purification was carried out by preparative SFC (Chiralpak®-IG-3, 30×250 mm×5 μm, temp 30° C., Back Pressure 100 bar, flow rate: 60 g/min, 70% CO₂ with 30% MeOH modifier) to provide Example 49 (20 mg) as Peak 1 and Example 50 (21 mg) as Peak 2. Example 49: ¹H NMR (400 MHz; DMSO-d₆) δ: 12.10 (br s, 1H), 7.22-7.11 (m, 4H), 7.05 (d, 2H), 6.78 (d, 2H), 6.36 (dd, 1H), 5.92 (d, 1H), 3.99 (dd, 1H), 3.16 (dd, 1H), 2.80 (s, 3H), 2.73 (dd, 1H), 2.45 (t, 2H), 2.27 (t, 2H), 1.78-1.71 (m, 2H), 1.31 (s, 3H), 1.09 (s, 3H). LCMS (Method A): 463.3 [M+H]⁺. Example 50: ¹H NMR (400 MHz; DMSO-d₆) δ: 12.10 (br s, 1H), 7.22-7.11 (m, 4H), 7.05 (d, 2H), 6.78 (d, 2H), 6.36 (dd, 1H), 5.92 (d, 1H), 3.99 (dd, 1H), 3.16 (dd, 1H), 2.80 (s, 3H), 2.73 (dd, 1H), 2.45 (t, 2H), 2.27 (t, 2H), 1.78-1.71 (m, 2H), 1.32 (s, 3H), 1.09 (s, 3H). LCMS (Method A): 463.3 [M+H]⁺.

### Example 51: N-((1S)-1-(4-((1,1-Dimethyl-2,3-dihydro-1H-inden-2-yl)amino)phenyl)-2,2,2-trifluoroethyl)-N-methyl-1,2,5-oxadiazole-3-carboxamide

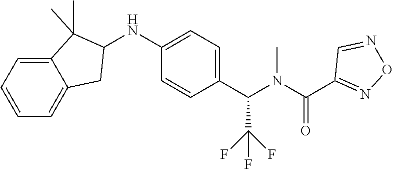 [SMILES: CN(C(=O)c1cnon1)[C@@H](c1ccc(NC2Cc3ccccc3C2(C)C)cc1)C(F)(F)F]

The title compound was prepared in an analogous manner to Intermediate 4 from Intermediate 14 (0.13 g, 0.34 mmol), pyridine (1.3 mL), POCl₃ (0.16 g, 1.01 mmol) and 1,2,5-oxadiazole-3-carboxylic acid (58 mg, 0.51 mmol, CAS 88598-08-7) at RT for 2 h. Purified by column chromatography (silica gel, eluting 10% EtOAc in petroleum ether). Chiral purification was carried out by preparative SFC (Chiralcel-OJ-3, 30×250 mm×5 μm, temp 30° C., Back Pressure 100 bar, flow rate: 100 g/min, 80% CO₂ with 20% MeCN modifier) to provide Example 51 (7.6 mg) as Peak 2. ¹H NMR (400 MHz; DMSO-d₆) δ: 9.41 (d, 1H), 7.22-7.12 (m, 6H), 6.82 (d, 2H), 6.43 (dd, 1H), 6.04 (d, 1H), 4.01 (dd, 1H), 3.18 (dd, 1H), 3.01 (s, 3H), 2.73 (dd, 1H), 1.32 (s, 3H), 1.09 (s, 3H). LCMS (Method A): 445.4 [M+H]⁺.

### Example 52: 4-(((1S)-1-(4-((1,1-Dimethyl-2,3-dihydro-1H-inden-2-yl)amino)phenyl)-2,2,2-trifluoroethyl)(methyl)amino)-4-oxobutanoic Acid—Isomer 1

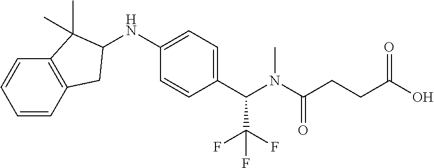 [SMILES: CN(C(=O)CCC(=O)O)[C@@H](c1ccc(NC2Cc3ccccc3C2(C)C)cc1)C(F)(F)F]

### Example 53: 4-(((1S)-1-(4-((1,1-Dimethyl-2,3-dihydro-1H-inden-2-yl)amino)phenyl)-2,2,2-trifluoroethyl)(methyl)amino)-4-oxobutanoic Acid—Isomer 2

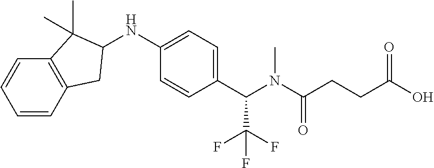 [SMILES: CN(C(=O)CCC(=O)O)[C@@H](c1ccc(NC2Cc3ccccc3C2(C)C)cc1)C(F)(F)F]

The title compounds were prepared from Intermediate 39 (0.24 g, 0.49 mmol) in THF/water/MeOH (4:1:1, 6 mL) treated with lithium hydroxide monohydrate (60 mg, 2.62 mmol) and stirred at RT for 2 h. The mixture was concentrated, diluted with water and washed with diethyl ether. The aqueous layer was acidified with citric acid then extracted with EtOAc. The organic layer was dried over Na₂SO₄, filtered and concentrated under reduced pressure. Purified by preparative HPLC (XBridge C18, 19×250 mm×5 μm, flow rate: 17 mL/min, 10 mM aqueous NH₄HCO₃ with MeCN 40% over 21 min). Chiral purification was carried out by preparative SFC (Chiralpak®-AD-H, 30×250 mm×5 μm, temp 30° C., Back Pressure 100 bar, flow rate: 100 g/min, 75% CO₂ with 25% MeOH modifier) to provide Example 52 (9.6 mg) as Peak 1 and Example 53 (9.5 mg) as Peak 2. Example 52: ¹H NMR (400 MHz; DMSO-d₆) δ: 12.15 (br s, 1H), 7.22-7.11 (m, 4H), 7.04 (d, 2H), 6.78 (d, 2H), 6.32 (dd, 1H), 5.92 (d, 1H), 3.99 (dd, 1H), 3.17 (dd, 1H), 2.84 (s, 3H), 2.74 (dd, 1H), 2.64-2.60 (m, 2H), 2.47-2.41 (m, 2H), 1.31 (s, 3H), 1.09 (s, 3H). LCMS (Method F): 449.3 [M+H]⁺. Example 53: ¹H NMR (400 MHz; DMSO-d₆) δ: 12.00 (br s, 1H), 7.22-7.11 (m, 4H), 7.04 (d, 2H), 6.78 (d, 2H), 6.32 (dd, 1H), 5.92 (d, 1H), 3.99 (dd, 1H), 3.17 (dd, 1H), 2.84 (s, 3H), 2.74 (dd, 1H), 2.64-2.60 (m, 2H), 2.47-2.41 (m, 2H), 1.32 (s, 3H), 1.09 (s, 3H). LCMS (Method F): 449.3 [M+H]⁺.

### Example 54: N-Methyl-N-((1S)-2,2,2-trifluoro-1-(4-((5-methoxy-2,3-dihydro-1H-inden-2-yl)amino)phenyl)ethyl)tetrahydro-2H-thiopyran-4-carboxamide 1,1-dioxide

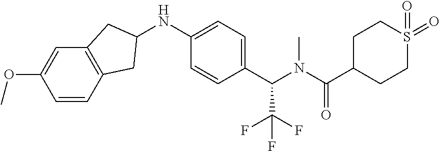 [SMILES: COc1ccc2c(c1)CC(Nc1ccc([C@H](N(C)C(=O)C3CCS(=O)(=O)CC3)C(F)(F)F)cc1)C2]

The title compound was prepared in an analogous manner to Intermediate 11 from Intermediate 3 (0.40 g, 0.94 mmol), 5-methoxy-2,3-dihydro-1H-inden-2-amine (0.15 g, 0.94 mmol, CAS 73305-09-6), Cs₂CO₃ (0.81 g, 2.81 mmol), tBuBrettphosPdG3 (79 mg, 0.09 mmol) and RuPhos (87 mg, 0.19 mmol) in 1,4-dioxane (5 mL) at 120° C. for 2 h. Purified by preparative HPLC (XBridge C18, 19×250 mm×5 μm, flow rate: 17 mL/min, 10 mM aqueous NH₄HCO₃ with MeCN 35% to 75% over 7 min, held at 75% for 3.5 min, ramped to 98% over 0.1 min and held for 4.4 min). Chiral purification was carried out by preparative SFC (Chiralpak®-AD-H, 30×250 mm×5 μm, temp 30° C., Back Pressure 110 bar, flow rate: 100 g/min, 75% CO₂ with 25% MeCN/IPA 1:1 modifier) to provide Example 54 (12 mg) as Peak 2. ¹H NMR (400 MHz; DMSO-d₆) δ: 7.16-7.06 (m, 3H), 6.82 (s, 1H), 6.71 (dd, 1H), 6.65 (d, 2H), 6.35 (dd, 1H), 6.20 (d, 1H), 4.20 (dd, 1H), 3.71 (s, 3H), 3.28-3.07 (m, 7H), 2.88 (s, 3H), 2.80-2.68 (m, 2H), 2.10-1.98 (m, 4H). LCMS (Method A): 511.5 [M+H]⁺.

### Example 55: N-((1S)-1-(4-((5-Chloro-2,3-dihydro-1H-inden-2-yl)amino)phenyl)-2,2,2-trifluoroethyl)-N-methyltetrahydro-2H-thiopyran-4-carboxamide 1,1-dioxide—Isomer 1

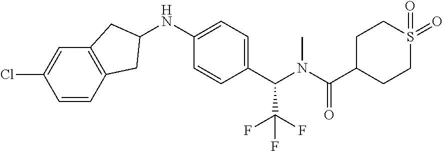 [SMILES: CN(C(=O)C1CCS(=O)(=O)CC1)[C@@H](c1ccc(NC2Cc3ccc(Cl)cc3C2)cc1)C(F)(F)F]

### Example 56: N-((1S)-1-(4-((5-Chloro-2,3-dihydro-1H-inden-2-yl)amino)phenyl)-2,2,2-trifluoroethyl)-N-methyltetrahydro-2H-thiopyran-4-carboxamide 1,1-dioxide—Isomer 2

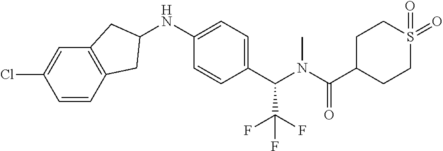 [SMILES: CN(C(=O)C1CCS(=O)(=O)CC1)[C@@H](c1ccc(NC2Cc3ccc(Cl)cc3C2)cc1)C(F)(F)F]

The title compounds were prepared in an analogous manner to Intermediate 11 from Intermediate 3 (0.30 g, 0.70 mmol), 5-chloro-2,3-dihydro-1H-inden-2-amine (0.15 g, 0.70 mmol, CAS 73536-83-1), Cs₂CO₃ (0.68 g, 2.11 mmol), tBuBrettphosPdG3 (59 mg, 0.07 mmol) and RuPhos (65 mg, 0.14 mmol) in 1,4-dioxane (5 mL) at 120° C. for 2 h. Purified by preparative HPLC (XBridge C18, 19×250 mm×5 μm, flow rate: 18 mL/min, 10 mM aqueous NH₄HCO₃ with MeCN 5% to 85% over 5 min, held at 85% for 10 min, ramped to 100% over 0.1 min and held for 2.9 min). Chiral purification was carried out by preparative SFC (Chiralpak®-AD-H, 21×250 mm×5 μm, temp 30° C., Back Pressure 100 bar, flow rate: 110 g/min, 80% CO₂ with 20% IPA/MeCN 1:1 modifier) to provide Example 55 (34 mg) as Peak 1 and Example 56 (23 mg) as Peak 2. Example 55: ¹H NMR (400 MHz; DMSO-d₆) δ: 7.30-7.16 (m, 3H), 7.08 (d, 2H), 6.65 (d, 2H), 6.35 (dd, 1H), 6.22 (d, 1H), 4.23 (dd, 1H), 3.29-3.07 (m, 7H), 2.89 (s, 3H), 2.83-2.73 (m, 2H), 2.10-1.95 (m, 4H). LCMS (Method A): 515.1 [M+H]⁺. Example 56: ¹H NMR (400 MHz; DMSO-d₆) δ: 7.30-7.16 (m, 3H), 7.08 (d, 2H), 6.65 (d, 2H), 6.35 (dd, 1H), 6.22 (d, 1H), 4.23 (dd, 1H), 3.29-3.07 (m, 7H), 2.88 (s, 3H), 2.83-2.73 (m, 2H), 2.10-1.95 (m, 4H). LCMS (Method A): 515.3 [M+H]⁺.

### Example 57: 1-Acetylazetidin-3-yl ((1S)-1-(4-((1,1-dimethyl-2,3-dihydro-1H-inden-2-yl)amino)phenyl)-2,2,2-trifluoroethyl)(methyl)carbamate—Isomer 1

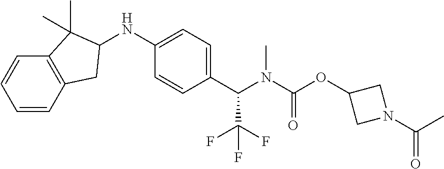 [SMILES: CC(=O)N1CC(OC(=O)N(C)[C@@H](c2ccc(NC3Cc4ccccc4C3(C)C)cc2)C(F)(F)F)C1]

### Example 58: 1-Acetylazetidin-3-yl ((1S)-1-(4-((1,1-dimethyl-2,3-dihydro-1H-inden-2-ylamino)phenyl)-2,2,2-trifluoroethyl)(methyl)carbamate—Isomer 2

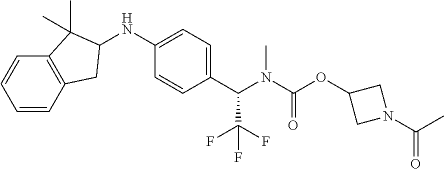 [SMILES: CC(=O)N1CC(OC(=O)N(C)[C@@H](c2ccc(NC3Cc4ccccc4C3(C)C)cc2)C(F)(F)F)C1]

To a stirred mixture of Intermediate 14 (0.30 g, 0.78 mmol) in DCM (5 mL) was added triphosgene (0.24 g, 0.82 mmol) portionwise at 0° C. and stirred for 30 min. Triethylamine (0.22 mL, 1.56 mmol) was added and stirred at RT for 2 h. The mixture was quenched with ice cold water, then extracted with DCM. The organic layer was dried over Na₂SO₄, filtered and concentrated under reduced pressure. The crude product was purified by flash chromatography (silica gel, eluting 20% EtOAc in petroleum ether). The material was then dissolved in THF (3 mL) and added to a mixture of 1-(3-hydroxyazetidin-1-yl)ethan-1-one (63 mg, 0.55 mmol, CAS 118972-96-6) and sodium hydride (60% in mineral oil, 30 mg, 0.73 mmol) in THF (5 mL) that was pre-mixed for 30 min at 0° C. The mixture was then stirred at RT for 2 h. The mixture was quenched with water and extracted with EtOAc. The organic layer was dried over Na₂SO₄, filtered and concentrated under reduced pressure. Purified by preparative HPLC (XBridge C18, 19×250 mm×5 μm, flow rate: 17 mL/min, 10 mM aqueous NH₄HCO₃ with MeCN 30% to 70% over 34 min, ramped to 95% over 0.1 min and held for 2 min). Chiral purification was carried out by preparative SFC (ChiralCel-OD-H, 30×250 mm×5 μm, temp 30° C., Back Pressure 100 bar, flow rate: 100 g/min, 85% CO₂ with 15% MeOH modifier) to provide Example 57 (7.8 mg) as Peak 1 and Example 58 (6.9 mg) as Peak 2. Example 57: ¹H NMR (400 MHz; DMSO-d₆) δ: 7.22-7.09 (m, 6H), 6.79 (d, 2H), 5.98 (d, 1H), 5.88 (br s, 1H), 5.19-5.13 (m, 1H), 4.48-4.42 (m, 1H), 4.18-4.06 (m, 2H), 4.00 (dd, 1H), 3.83-3.75 (m, 1H), 3.17 (dd, 1H), 2.78-2.65 (m, 4H), 1.77 (s, 3H), 1.31 (s, 3H), 1.09 (s, 3H). LCMS (Method A): 490.2 [M+H]⁺. Example 58: ¹H NMR (400 MHz; DMSO-d₆) δ: 7.22-7.09 (m, 6H), 6.79 (d, 2H), 5.98 (d, 1H), 5.88 (br s, 1H), 5.19-5.13 (m, 1H), 4.48-4.42 (m, 1H), 4.18-4.06 (m, 2H), 4.00 (dd, 1H), 3.83-3.75 (m, 1H), 3.17 (dd, 1H), 2.78-2.65 (m, 4H), 1.77 (s, 3H), 1.32 (s, 3H), 1.09 (s, 3H). LCMS (Method A): 490.5 [M+H]⁺.

### Example 59: tert-Butyl 3-((((1S)-1-(4-((1,1-dimethyl-2,3-dihydro-1H-inden-2-yl)amino)phenyl)-2,2,2-trifluoroethyl)(methyl)carbamoyl)oxy)azetidine-1-carboxylate

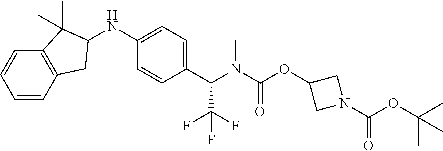 [SMILES: CN(C(=O)OC1CN(C(=O)OC(C)(C)C)C1)[C@@H](c1ccc(NC2Cc3ccccc3C2(C)C)cc1)C(F)(F)F]

The title compound was prepared in an analogous manner to Intermediate 11 from Intermediate 41 (0.75 g, 1.61 mmol), 1,1-dimethyl-2,3-dihydro-1H-inden-2-amine hydrochloride (0.32 g, 1.61 mmol, CAS 74413-86-8), Cs₂CO₃ (1.04 g, 3.20 mmol), tBuBrettphosPdG3 (0.14 g, 0.16 mmol) and RuPhos (0.15 g, 0.32 mmol) in 1,4-dioxane (10 mL) at 160° C. for 2 h. Purified by preparative HPLC (XBridge C18, 19×250 mm×5 μm, flow rate: 18 mL/min, 10 mM aqueous NH₄HCO₃ with MeCN 55% to 75% over 7 min, held at 75% for 7 min, ramped to 95% over 0.1 min and held for 3.9 min) to provide Example 59 (0.135 g). ¹H NMR (400 MHz; DMSO-d₆) δ: 7.22-7.08 (m, 6H), 6.79 (d, 2H), 5.97 (d, 1H), 5.88 (br s, 1H), 5.17-5.10 (m, 1H), 4.20-4.13 (m, 2H), 3.99 (dd, 1H), 3.93-3.75 (m, 2H), 3.17 (dd, 1H), 2.77-2.65 (m, 4H), 1.38 (s, 9H), 1.31 (s, 3H), 1.09 (s, 3H). LCMS (Method A): 548.2 [M+H]⁺.

### Example 60: Azetidin-3-yl ((1S)-1-(4-((1,1-dimethyl-2,3-dihydro-1H-inden-2-ylamino)phenyl)-2,2,2-trifluoroethyl)(methyl)carbamate Hydrochloride

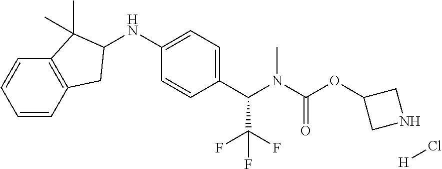

The title compound was prepared from Example 59 (50 mg, 0.09 mmol) in EtOAc (2 mL) at 0° C. treated with 4M HCl in 1,4-dioxane (2 mL, 2.0 mmol) then stirred at RT for 2 h. The mixture was concentrated under reduced pressure then the solid was triturated with diethyl ether and the solid dried under reduced pressure. Purified by preparative HPLC (Sunfire C18, 19×150 mm×5 μm, flow rate: 16 mL/min, 0.05% HCl in water with MeCN 10% to 40% over 9 min, held at 40% for 6 min, ramped to 98% over 0.1 min and held for 1.9 min) to provide Example 60 (24 mg). ¹H NMR (400 MHz; DMSO-d₆) δ: 9.11 (br s, 1H), 8.85 (br s, 1H), 7.22-7.08 (m, 6H), 6.80 (d, 2H), 5.99 (br s, 1H), 5.89-5.80 (m, 1H), 5.27-5.20 (m, 1H), 4.32-4.23 (m, 2H), 4.11-3.98 (m, 3H), 3.17 (dd, 1H), 2.80-2.65 (m, 4H), 1.32 (s, 3H), 1.09 (s, 3H). LCMS (Method A): 448.2 [M+H]⁺.

### Example 61: N-((1S)-1-(4-((1,1-Dimethyl-2,3-dihydro-1H-inden-2-yl)amino)phenyl)-2,2,2-trifluoroethyl)-N-methyl-3-(N-methylsulfamoyl)propanamide—Isomer 1

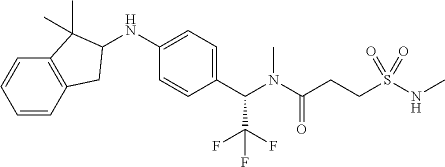 [SMILES: CNS(=O)(=O)CCC(=O)N(C)[C@@H](c1ccc(NC2Cc3ccccc3C2(C)C)cc1)C(F)(F)F]

### Example 62: N-((1S)-1-(4-((1,1-Dimethyl-2,3-dihydro-1H-inden-2-yl)amino)phenyl)-2,2,2-trifluoroethyl)-N-methyl-3-(N-methylsulfamoyl)propanamide—Isomer 2

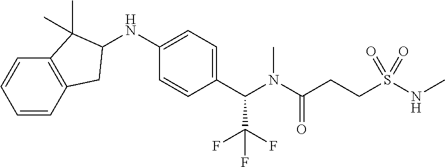 [SMILES: CNS(=O)(=O)CCC(=O)N(C)[C@@H](c1ccc(NC2Cc3ccccc3C2(C)C)cc1)C(F)(F)F]

The title compounds were prepared in an analogous manner to Intermediate 4 from Intermediate 14 (0.22 g, 0.57 mmol), pyridine (4.4 mL), DMAP (7 mg, 0.06 mmol), POCl₃ (0.16 mL, 1.71 mmol) and 3-(N-methylsulfamoyl)propanoic acid (0.19 g, 1.14 mmol, CAS 933714-87-5) at RT for 2 h. Purified by column chromatography (silica gel, eluting 40% EtOAc in petroleum ether). Further purified by preparative HPLC (XBridge C18, 19×250 mm×5 μm, flow rate: 18 mL/min, 10 mM aqueous NH₄HCO₃ with MeCN 40% to 70% over 9 min, held at 70% for 6 min, ramped to 98% over 0.1 min and held for 2.9 min). Chiral purification was carried out by preparative SFC (Chiralcel-OJ-3, 30×250 mm×5 μm, temp 30° C., Back Pressure 100 bar, flow rate: 100 g/min, 80% CO₂ with 20% MeCN modifier) to provide Example 61 (14 mg) as Peak 1 and Example 62 (14 mg) as Peak 2. Example 61: ¹H NMR (400 MHz; DMSO-d₆) δ: 7.22-7.12 (m, 4H), 7.07 (d, 2H), 6.96 (dd, 1H), 6.78 (d, 2H), 6.32 (dd, 1H), 5.94 (d, 1H), 3.99 (dd, 1H), 3.30-3.28 (m, 2H), 3.18 (dd, 1H), 2.88-2.65 (m, 6H), 2.56 (d, 3H), 1.32 (s, 3H), 1.09 (s, 3H). LCMS (Method A): 498.2 [M+H]⁺. Example 62: ¹H NMR (400 MHz; DMSO-d₆) δ: 7.22-7.12 (m, 4H), 7.07 (d, 2H), 6.96 (dd, 1H), 6.78 (d, 2H), 6.32 (dd, 1H), 5.94 (d, 1H), 3.99 (dd, 1H), 3.30-3.28 (m, 2H), 3.17 (dd, 1H), 2.88-2.65 (m, 6H), 2.56 (d, 3H), 1.31 (s, 3H), 1.09 (s, 3H). LCMS (Method A): 498.2 [M+H]⁺.

### Example 63: N-((1R)-1-(4-((1,1-Dimethyl-2,3-dihydro-1H-inden-2-yl)amino)phenyl)ethyl)-N-methyltetrahydro-2H-thiopyran-4-carboxamide 1,1-dioxide

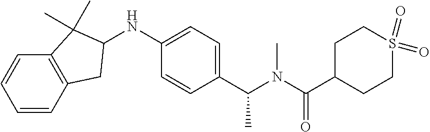 [SMILES: C[C@H](c1ccc(NC2Cc3ccccc3C2(C)C)cc1)N(C)C(=O)C1CCS(=O)(=O)CC1]

The title compound was prepared in an analogous manner to Intermediate 11 from Intermediate 42 (0.60 g, 1.60 mmol), 1,1-dimethyl-2,3-dihydro-1H-inden-2-amine hydrochloride (0.24 g, 1.60 mmol, CAS 74413-86-8), Cs₂CO₃ (1.56 g, 4.80 mmol), tBuBrettphosPdG3 (0.14 g, 0.16 mmol) and RuPhos (0.15 g, 0.32 mmol) in 1,4-dioxane (6 mL) at 140° C. for 1 h. Purified by column chromatography (silica gel, eluting 50-55% EtOAc in petroleum ether). Further purified preparative HPLC (XBridge C18, 19×250 mm×5 μm, flow rate: 18 mL/min, 10 mM aqueous NH₄HCO₃ with MeCN 35% to 75% over 7 min, held at 75% for 4 min, ramped to 98% over 0.1 min and held for 5.9 min). Chiral purification was carried out by preparative SFC (Chiralcel-OJ-H, 30×250 mm×5 μm, temp 30° C., Back Pressure 100 bar, flow rate: 110 g/min, 70% CO₂ with 30% MeOH modifier) to provide Example 63 (21 mg) as Peak 1. ¹H NMR (400 MHz; DMSO-d₆) δ: 7.21-7.11 (m, 4H), 6.98 (d, 1H), 6.93 (d, 1H), 6.74 (d, 1H), 6.70 (d, 1H), 5.69 (dd, 1H), 5.58 (d, 1H), 4.01-3.92 (m, 1H), 3.29-3.04 (m, 5H), 3.03-2.96 (m, 1H), 2.75-2.68 (m, 1H), 2.55 (s, 3H), 2.08-1.96 (m, 4H), 1.49 (d, 2H), 1.35-1.31 (m, 4H), 1.08 (s, 3H). LCMS (Method A): 455.5 [M+H]⁺.

### Example 64: N-((1S)-1-(4-((1,1-Dimethyl-2,3-dihydro-1H-inden-2-yl)amino)phenyl)-2,2,2-trifluoroethyl)-N-methyl-4-(N-methylsulfamoyl)butanamide—Isomer 1

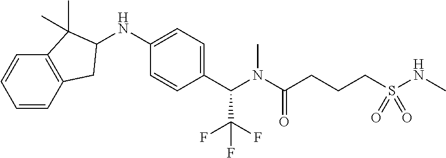 [SMILES: CNS(=O)(=O)CCCC(=O)N(C)[C@@H](c1ccc(NC2Cc3ccccc3C2(C)C)cc1)C(F)(F)F]

### Example 65: N-((1S)-1-(4-((1,1-Dimethyl-2,3-dihydro-1H-inden-2-yl)amino)phenyl)-2,2,2-trifluoroethyl)-N-methyl-4-(N-methylsulfamoyl)butanamide—Isomer 2

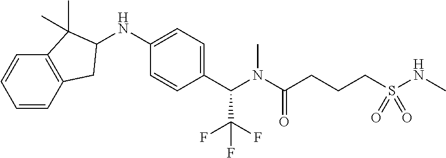 [SMILES: CNS(=O)(=O)CCCC(=O)N(C)[C@@H](c1ccc(NC2Cc3ccccc3C2(C)C)cc1)C(F)(F)F]

The title compounds were prepared in an analogous manner to Intermediate 3 from Intermediate 14 (0.15 g, 0.39 mmol), pyridine (0.1 mL, 1.17 mmol), T3P® (50% EtOAc, 1.15 mL, 1.95 mmol) and 4-(N-methylsulfamoyl)butanoic acid (0.11 g, 0.58 mmol, CAS 1042583-67-4) at RT for 16 h. Purified by preparative HPLC (XBridge C18, 19×250 mm×5 μm, flow rate: 17 mL/min, 10 mM aqueous NH₄HCO₃ with MeCN 4% to 60% over 10 min, held at 60% for 7 min, ramped to 98% over 1 min and held for 2 min). Chiral purification was carried out by preparative SFC (Chiralcel-OJ-H, 21×250 mm×5 μm, temp 30° C., Back Pressure 100 bar, flow rate: 60 g/min, 80% CO₂ with 20% IPA/MeCN 1:1 modifier) to provide Example 64 (12 mg) as Peak 1 and Example 65 (12 mg) as Peak 2. Example 64: ¹H NMR (400 MHz; DMSO-d₆) δ: 7.22-7.12 (m, 4H), 7.06 (d, 2H), 6.94 (dd, 1H), 6.78 (d, 2H), 6.36 (dd, 1H), 5.93 (d, 1H), 3.99 (dd, 1H), 3.16 (dd, 1H), 3.08-3.02 (m, 2H), 2.81 (s, 3H), 2.73 (dd, 1H), 2.65-2.60 (m, 2H), 2.56 (d, 3H), 1.94-1.86 (m, 2H), 1.32 (s, 3H), 1.09 (s, 3H). LCMS (Method A): 512.5 [M+H]⁺. Example 65: ¹H NMR (400 MHz; DMSO-d₆) δ: 7.22-7.12 (m, 4H), 7.06 (d, 2H), 6.94 (dd, 1H), 6.78 (d, 2H), 6.36 (dd, 1H), 5.93 (d, 1H), 3.99 (dd, 1H), 3.16 (dd, 1H), 3.08-3.02 (m, 2H), 2.80 (s, 3H), 2.73 (dd, 1H), 2.65-2.60 (m, 2H), 2.56 (d, 3H), 1.94-1.86 (m, 2H), 1.31 (s, 3H), 1.09 (s, 3H). LCMS (Method A): 512.5 [M+H]⁺.

### Example 66: N-((1S)-1-(4-((1,1-Dimethyl-2,3-dihydro-1H-inden-2-yl)amino)phenyl)-2,2,2-trifluoroethyl)-2-(2,4-dioxoimidazolidin-1-yl)-N-methylacetamide—Isomer 1

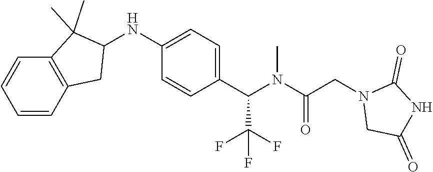 [SMILES: CN(C(=O)CN1CC(=O)NC1=O)[C@@H](c1ccc(NC2Cc3ccccc3C2(C)C)cc1)C(F)(F)F]

### Example 67: N-((1S)-1-(4-((1,1-Dimethyl-2,3-dihydro-1H-inden-2-yl)amino)phenyl)-2,2,2-trifluoroethyl)-2-(2,4-dioxoimidazolidin-1-yl)-N-methylacetamide—Isomer 2

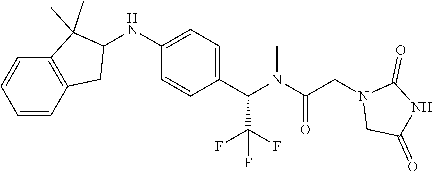 [SMILES: CN(C(=O)CN1CC(=O)NC1=O)[C@@H](c1ccc(NC2Cc3ccccc3C2(C)C)cc1)C(F)(F)F]

The title compounds were prepared in an analogous manner to Intermediate 3 from Intermediate 14 (0.15 g, 0.39 mmol), pyridine (0.13 mL, 1.56 mmol), T3P® (50% EtOAc, 1.74 mL, 2.73 mmol) and 2-(2,4-dioxoimidazolidin-1-yl)acetic acid (80 mg, 0.51 mmol, CAS 94738-31-5) at RT for 12 h. Purified by preparative HPLC (XBridge 018, 19×250 mm×5 μm, flow rate: 17 mL/min, 10 mM aqueous NH₄HCO₃ with MeCN 30% to 60% over 6 min, held at 60% for 8.6 min, ramped to 98% over 0.1 min and held for 3 min) Chiral purification was carried out by preparative SFC (Chiralcel-OJ-H, 21×250 mm×5 μm, temp 30° C. Back Pressure 100 bar, flow rate: 110 g/min, 85% CO₂ with 15% IPA/MeCN 1:1 modifier) to provide Example 66 (22 mg) as Peak 1 and Example 67 (23 mg) as Peak 2. Example 66: ¹H NMR (400 MHz; DMSO-d₆) δ: 10.91 (br s, 1H), 7.22-7.12 (m, 4H), 7.08 (d, 2H), 6.78 (d, 2H), 6.24 (dd, 1H), 5.96 (d, 1H), 4.50-4.25 (m, 2H), 3.99 (dd, 1H), 3.92 (d, 2H), 3.16 (dd, 1H), 2.84 (s, 3H), 2.73 (dd, 1H), 1.32 (s, 3H), 1.09 (s, 3H). LCMS (Method A): 489.4 [M+H]⁺. Example 67: ¹H NMR (400 MHz; DMSO-d₆) δ: 10.91 (br s, 1H), 7.21-7.12 (m, 4H), 7.08 (d, 2H), 6.79 (d, 2H), 6.24 (dd, 1H), 5.96 (d, 1H), 4.50-4.25 (m, 2H), 4.00 (dd, 1H), 3.88 (d, 2H), 3.17 (dd, 1H), 2.83 (s, 3H), 2.73 (dd, 1H), 1.31 (s, 3H), 1.09 (s, 3H). LCMS (Method A): 489.4 [M+H]⁺.

### Example 68: N-((1S)-1-(4-((1,1-Dimethyl-2,3-dihydro-1H-inden-2-yl)amino)phenyl)-2,2,2-trifluoroethyl)-N-methyl-1-(methylsulfonyl)azetidine-3-carboxamide—Isomer 1

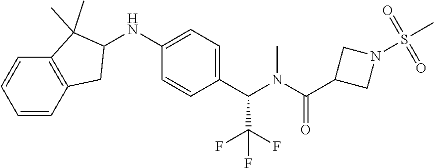 [SMILES: CN(C(=O)C1CN(S(C)(=O)=O)C1)[C@@H](c1ccc(NC2Cc3ccccc3C2(C)C)cc1)C(F)(F)F]

### Example 69: N-((1S)-1-(4-((1,1-Dimethyl-2,3-dihydro-1H-inden-2-yl)amino)phenyl)-2,2,2-trifluoroethyl)-N-methyl-1-(methylsulfonyl)azetidine-3-carboxamide—Isomer 2

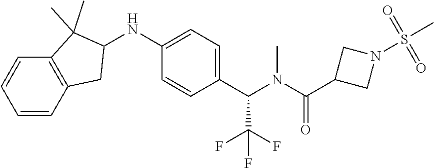 [SMILES: CN(C(=O)C1CN(S(C)(=O)=O)C1)[C@@H](c1ccc(NC2Cc3ccccc3C2(C)C)cc1)C(F)(F)F]

The title compounds were prepared in an analogous manner to Intermediate 11 from Intermediate 43 (0.25 g, 0.58 mmol), 1,1-dimethyl-2,3-dihydro-1H-inden-2-amine hydrochloride (0.14 g, 0.69 mmol, CAS 74413-86-8), Cs₂CO₃ (0.57 g, 1.74 mmol), tBuBrettphosPdG3 (50 mg, 0.06 mmol) and RuPhos (54 mg, 0.12 mmol) in 1,4-dioxane/DMF (9:1, 5 mL) at 140° C. for 2 h. Purified by preparative HPLC (XBridge Phenyl, 19×250 mm×5 μm, flow rate: 17 mL/min, 10 mM aqueous NH₄HCO₃ with MeCN 30% to 70% over 6 min, held at 70% for 6.8 min, ramped to 98% over 0.1 min and held for 3.6 min). Chiral purification was carried out by preparative SFC (Chiralcel-OJ-H, 21×250 mm×5 μm, temp 30° C., Back Pressure 100 bar, flow rate: 100 g/min, 80% CO₂ with 20% MeCN modifier) to provide Example 68 (21 mg) as Peak 1 and Example 69 (25 mg) as Peak 2. Example 68: ¹H NMR (400 MHz; DMSO-d₆) δ: 7.22-7.11 (m, 4H), 7.07 (d, 2H), 6.79 (d, 2H), 6.31 (dd, 1H), 5.96 (d, 1H), 4.12-3.95 (m, 5H), 3.93-3.86 (m, 1H), 3.16 (dd, 1H), 3.02 (s, 3H), 2.76-2.65 (m, 4H), 1.32 (s, 3H), 1.09 (s, 3H). LCMS (Method A): 510.4 [M+H]⁺. Example 69: ¹H NMR (400 MHz; DMSO-d₆) δ: 7.22-7.11 (m, 4H), 7.07 (d, 2H), 6.79 (d, 2H), 6.31 (dd, 1H), 5.96 (d, 1H), 4.12-3.95 (m, 5H), 3.93-3.86 (m, 1H), 3.16 (dd, 1H), 3.00 (s, 3H), 2.76-2.65 (m, 4H), 1.31 (s, 3H), 1.09 (s, 3H). LCMS (Method A): 510.4 [M+H]⁺.

### Example 70: (S)—N-(1-(4-((2,3-Dihydro-1H-inden-2-yl)amino)phenyl)-2,2,2-trifluoroethyl)-N-methylpiperidine-4-carboxamide

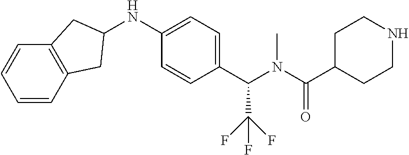 [SMILES: CN(C(=O)C1CCNCC1)[C@@H](c1ccc(NC2Cc3ccccc3C2)cc1)C(F)(F)F]

The title compound was prepared from Intermediate 45 (1.17 g, 2.20 mmol) in EtOAc (11.7 mL) at 0° C. treated with 4M HCl in 1,4-dioxane (11.7 mL, 46.8 mmol) then stirred at RT for 2 h. The mixture was concentrated under reduced pressure. Purified by preparative HPLC (XBridge C18, 19×150 mm×5 μm, flow rate: 18 mL/min, 10 mM aqueous NH₄HCO₃ plus 0.2% NH₄OH with MeCN 30% to 65% over 7 min, held at 65% for 6 min, ramped to 100% over 0.1 min and held for 2.4 min). Chiral purification was carried out by preparative SFC (Chiralpak®-AD-H, 30×250 mm×5 μm, temp 30° C., Back Pressure 100 bar, flow rate: 100 g/min, 60% CO₂ with 40% (0.2% methanolic ammonia in 1:1 MeOH/MeCN) modifier) to provide Example 70 (236 mg) as Peak 1. ¹H NMR (400 MHz; DMSO-d₆) δ: 7.25-7.20 (m, 2H), 7.18-7.12 (m, 2H), 7.05 (d, 2H), 6.66 (d, 2H), 6.38 (dd, 1H), 6.19 (d, 1H), 4.25-4.18 (m, 1H), 3.30-3.27 (m, 2H), 2.98-2.91 (m, 2H), 2.85 (s, 3H), 2.79 (dd, 2H), 2.75-2.60 (m, 2H), 2.49-2.45 (m, 1H), 2.01 (br s, 1H), 1.62-1.41 (m, 4H). LCMS (Method A): 432.5 [M+H]⁺.

### Example 71: (S)—N-(1-(5-((2,3-Dihydro-1H-inden-2-yl)amino)pyridin-2-yl)-2,2,2-trifluoroethyl)-N-methyltetrahydro-2H-thiopyran-4-carboxamide 1,1-dioxide

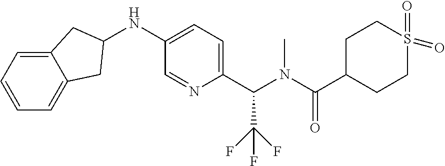 [SMILES: CN(C(=O)C1CCS(=O)(=O)CC1)[C@@H](c1ccc(NC2Cc3ccccc3C2)cn1)C(F)(F)F]

The title compound was prepared in an analogous manner to Intermediate 11 from Intermediate 48 (0.50 g, 1.17 mmol), 2,3-dihydro-1H-inden-2-amine hydrochloride (0.30 g, 1.75 mmol, CAS 2975-41-9), Cs₂CO₃ (1.14 g, 3.50 mmol), XPhosPdG2 (92 mg, 0.12 mmol) in toluene (5 mL) at 100° C. for 16 h. Purified by column chromatography (silica gel, eluting 35-40% EtOAc in petroleum ether). Further purified by preparative HPLC (XBridge C18, 19×250 mm×5 μm, flow rate: 18 mL/min, 10 mM aqueous NH₄HCO₃ with MeCN 40% to 60% over 7 min, held at 60% for 4.5 min, ramped to 100% over 0.1 min and held for 5.4 min). Chiral purification was carried out by preparative SFC (Chiralpak®-IC-H, 30×250 mm×5 μm, temp 30° C., Back Pressure 100 bar, flow rate: 100 g/min, 60% CO₂ with 40% MeOH modifier) to provide Example 71 (28 mg) as Peak 2. ¹H NMR (400 MHz; DMSO-d₆) δ: 8.06-8.02 (m, 1H), 7.26-7.21 (m, 2H), 7.18-7.12 (m, 2H), 7.10-7.01 (m, 2H), 6.53-6.45 (m, 1H), 6.37 (dd, 1H), 4.30-4.22 (m, 1H), 3.30-3.15 (m, 6H), 3.01 (s, 3H), 2.80 (dd, 2H), 2.60 (s, 1H), 2.19-1.94 (m, 4H). LCMS (Method A): 482.4 [M+H]⁺.

### Example 72: N-((1S)-1-(4-((1,1-Dimethyl-2,3-dihydro-1H-inden-2-yl)amino)phenyl)-2,2,2-trifluoroethyl)-N-methylazetidine-3-carboxamide Hydrochloride

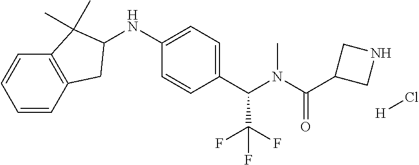

The title compound (26 mg) was prepared from Intermediate 50 (0.20 g, 0.37 mmol) in EtOAc (2 mL) at 0° C. treated with 4M HCl in 1,4-dioxane (1 mL, 4.0 mmol) then stirred at RT for 2 h. The mixture was concentrated under reduced pressure. Purified by preparative HPLC (Sunfire C18, 19×150 mm×5 μm, flow rate: 16 mL/min, 0.05% HCl in water with MeCN 10% to 30% over 1 min, then to 50% over 24 min, ramped to 95% over 0.1 min and held for 2 min). ¹H NMR (400 MHz; DMSO-d₆) δ: 9.10 (br s, 1H), 8.79 (br s, 1H), 7.22-7.05 (m, 6H), 6.79 (d, 2H), 6.30 (dd, 1H), 5.99 (br s, 1H), 4.21-3.96 (m, 6H), 3.20-3.12 (m, 1H), 2.68-2.66 (m, 4H), 1.32 (s, 3H), 1.09 (s, 3H). LCMS (Method A): 432.7 [M+H]⁺.

### Example 73: N³-((1S)-1-(4-((1,1-Dimethyl-2,3-dihydro-1H-inden-2-yl)amino)phenyl)-2,2,2-trifluoroethyl)-N¹,N³-dimethylazetidine-1,3-dicarboxamide—Isomer 1

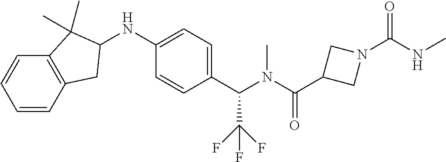 [SMILES: CNC(=O)N1CC(C(=O)N(C)[C@@H](c2ccc(NC3Cc4ccccc4C3(C)C)cc2)C(F)(F)F)C1]

### Example 74: N³-((1S)-1-(4-((1,1-Dimethyl-2,3-dihydro-1H-inden-2-yl)amino)phenyl)-2,2,2-trifluoroethyl)-N¹,N³-dimethylazetidine-1,3-dicarboxamide—Isomer 2

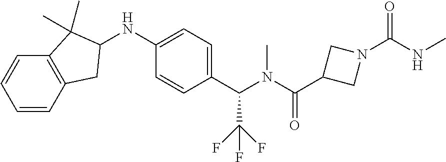 [SMILES: CNC(=O)N1CC(C(=O)N(C)[C@@H](c2ccc(NC3Cc4ccccc4C3(C)C)cc2)C(F)(F)F)C1]

To a stirred mixture of Example 72 (0.19 g, 0.63 mmol) in DCM (5 mL) was added triphosgene (0.19 g, 0.63 mmol) portionwise at 0° C. and stirred for 5 min. Triethylamine (0.27 mL, 1.87 mmol) was added and stirred at RT for 1 h. The mixture was concentrated under reduced pressure. The crude product was dissolved in DCM (10 mL) at 0° C. then methylamine (2M in THF, 0.82 mL, 1.64 mmol) and pyridine (0.14 mL, 1.64 mmol) were added and stirred at RT for 1 h. The mixture was concentrated under reduced pressure. Purified by column chromatography (silica gel, eluting 30-70% EtOAc in petroleum ether) and then by preparative HPLC (XBridge C18, 19×250 mm×5 μm, flow rate: 18 mL/min, 10 mM aqueous NH₄HCO₃ with MeCN 33% to 55% over 5 min, held at 55% for 14 min, ramped to 100% over 0.1 min and held for 4.9 min). Chiral purification was carried out by preparative SFC (Chiralpak®-IG3, 30×250 mm×5 μm, temp 30° C., Back Pressure 100 bar, flow rate: 80 g/min, 70% CO₂ with 30% MeOH/MeCN 1:1 modifier) to provide Example 73 (25 mg) as Peak 1 and Example 74 (23 mg) as Peak 2. Example 73: ¹H NMR (400 MHz; DMSO-d₆) δ: 7.22-7.12 (m, 4H), 7.06 (d, 2H), 6.79 (d, 2H), 6.37-6.26 (m, 2H), 5.97 (d, 1H), 4.03-3.90 (m, 3H), 3.88-3.74 (m, 3H), 3.16 (dd, 1H), 2.78-2.65 (m, 4H), 2.56-2.52 (m, 3H), 1.32 (s, 3H), 1.09 (s, 3H). LCMS (Method A): 489.5 [M+H]⁺. Example 74: ¹H NMR (400 MHz; DMSO-d₆) δ: 7.22-7.12 (m, 4H), 7.06 (d, 2H), 6.79 (d, 2H), 6.37-6.26 (m, 2H), 5.97 (d, 1H), 4.03-3.90 (m, 3H), 3.88-3.74 (m, 3H), 3.16 (dd, 1H), 2.78-2.65 (m, 4H), 2.56-2.52 (m, 3H), 1.31 (s, 3H), 1.09 (s, 3H). LCMS (Method A): 489.5 [M+H]⁺.

### Example 75: N-((1S)-1-(4-((1,1-Dimethyl-2,3-dihydro-1H-inden-2-yl)amino)phenyl)-2,2,2-trifluoroethyl)-N,1-dimethylazetidine-3-carboxamide—Isomer 1

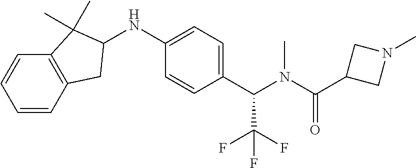 [SMILES: CN1CC(C(=O)N(C)[C@@H](c2ccc(NC3Cc4ccccc4C3(C)C)cc2)C(F)(F)F)C1]

### Example 76: N-((1S)-1-(4-((1,1-Dimethyl-2,3-dihydro-1H-inden-2-yl)amino)phenyl)-2,2,2-trifluoroethyl)-N,1-dimethylazetidine-3-carboxamide—Isomer 2

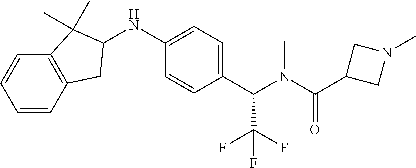 [SMILES: CN1CC(C(=O)N(C)[C@@H](c2ccc(NC3Cc4ccccc4C3(C)C)cc2)C(F)(F)F)C1]

To a stirred mixture of Example 72 (0.39 g, 0.83 mmol) in DCM (12 mL) was added AcOH (1 drop, catalytic) and paraformaldehyde (75 mg, 2.50 mmol) and stirred for 15 min at RT. Sodium triacetoxyborohydride (0.88 g, 4.16 mmol) was added at 0° C. then stirred at RT for 2 h. The mixture was diluted with water and extracted with DCM. The organic layer was dried over Na₂SO₄, filtered and concentrated under reduced pressure. Purified by preparative HPLC (XBridge C18, 19×250 mm×5 μm, flow rate: 18 mL/min, 10 mM aqueous NH₄HCO₃ with MeCN 50% to 70% over 9 min, held at 70% for 4 min, ramped to 100% over 0.1 min). Chiral purification was carried out by preparative SFC (Chiralpak®-IG3, 21×250 mm×5 μm, temp 30° C., Back Pressure 100 bar, flow rate: 80 g/min, 80% CO₂ with 20% (0.2% methanol ammonia in MeOH/MeCN 1:1) modifier to provide Example 75 (15 mg) as Peak 1 and Example 76 (13 mg) as Peak 2. Example 75: ¹H NMR (400 MHz; DMSO-d₆) δ: 7.22-7.12 (m, 4H), 7.05 (d, 2H), 6.78 (d, 2H), 6.31 (dd, 1H), 5.93 (d, 1H), 3.98 (dd, 1H), 3.58-3.408 (m, 3H), 3.16 (dd, 1H), 3.08 (t, 2H), 2.68-2.62 (m, 4H), 2.17 (s, 3H), 1.31 (s, 3H), 1.09 (s, 3H). LCMS (Method A): 446.5 [M+H]⁺. Example 76: ¹H NMR (400 MHz; DMSO-d₆) δ: 7.22-7.12 (m, 4H), 7.05 (d, 2H), 6.78 (d, 2H), 6.31 (dd, 1H), 5.93 (d, 1H), 3.98 (dd, 1H), 3.58-3.40 (m, 3H), 3.16 (dd, 1H), 3.08 (t, 2H), 2.68-2.62 (m, 4H), 2.17 (s, 3H), 1.31 (s, 3H), 1.08 (s, 3H). LCMS (Method A): 446.5 [M+H]⁺.

### Example 77: N-((1S)-1-(5-((1,1-Dimethyl-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

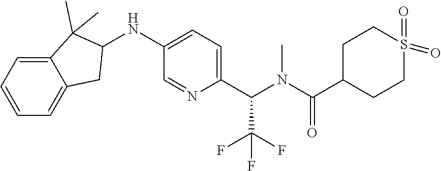 [SMILES: CN(C(=O)C1CCS(=O)(=O)CC1)[C@@H](c1ccc(NC2Cc3ccccc3C2(C)C)cn1)C(F)(F)F]

### Example 78: N-((1S)-1-(5-((1,1-Dimethyl-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

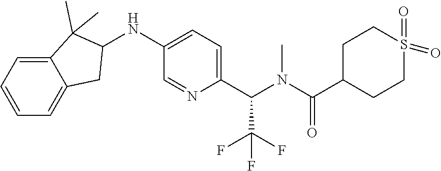 [SMILES: CN(C(=O)C1CCS(=O)(=O)CC1)[C@@H](c1ccc(NC2Cc3ccccc3C2(C)C)cn1)C(F)(F)F]

The title compound was prepared in an analogous manner to Intermediate 11 from Intermediate 48 (1.25 g, 2.91 mmol), 1,1-dimethyl-2,3-dihydro-1H-inden-2-amine hydrochloride (0.86 g, 4.37 mmol, CAS 74413-86-8), Cs₂CO₃ (2.84 g, 8.73 mmol), and RuPhosPdG3 (0.24 g, 0.29 mmol) in toluene (12.5 mL) at 110° C. for 16 h. Purified by column chromatography (silica gel, eluting 40-45% EtOAc in petroleum ether). Further purified preparative HPLC (X-Select C18, 19×250 mm×5 μm, flow rate: 16 mL/min, 10 mM aqueous NH₄HCO₃ with MeCN 30% to 60% over 6 min, held at 60% for 9 min, ramped to 98% over 0.1 min and held for 5.9 min). Chiral purification was carried out by preparative SFC (LUX-Cellulose-2, 30×250 mm×5 μm, temp 30° C., Back Pressure 100 bar, flow rate: 100 g/min, 70% CO₂ with 30% MeOH modifier) followed by a 2^{nd} purification (Chiracel-OJ-H, 30×250 mm×5 μm, temp 30° C., Back Pressure 100 bar, flow rate: 100 g/min, 75% CO₂ with 25% MeOH modifier) to provide Example 77 (18 mg) as Peak 2 and Example 78 (15 mg) as Peak 4. Example 77: ¹H NMR (400 MHz; DMSO-d₆) δ: 8.16-8.10 (m, 1H), 7.22-7.09 (m, 6H), 6.36 (dd, 1H), 6.28-6.20 (m, 1H), 4.04 (dd, 1H), 3.29-3.06 (m, 5H), 3.02 (s, 3H), 2.78-2.68 (m, 2H), 2.15-1.96 (m, 4H), 1.32 (s, 3H), 1.10 (s, 3H). LCMS (Method A): 510.6 [M+H]⁺. Example 78: ¹H NMR (400 MHz; DMSO-d₆) δ: 8.17-8.10 (m, 1H), 7.22-7.09 (m, 6H), 6.36 (dd, 1H), 6.28-6.20 (m, 1H), 4.04 (dd, 1H), 3.29-3.06 (m, 5H), 3.02 (s, 3H), 2.78-2.68 (m, 2H), 2.15-1.96 (m, 4H), 1.32 (s, 3H), 1.10 (s, 3H). LCMS (Method A): 510.5 [M+H]⁺.

### Example 79: N-((1S)-1-(4-((1,1-Dimethyl-2,3-dihydro-1H-inden-2-yl)amino)phenyl)-2,2,2-trifluoroethyl)-N-methylcyclopropanesulfonamide—Isomer 1

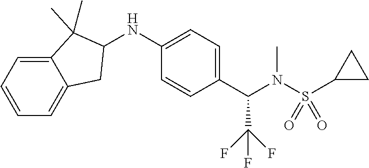 [SMILES: CN([C@@H](c1ccc(NC2Cc3ccccc3C2(C)C)cc1)C(F)(F)F)S(=O)(=O)C1CC1]

### Example 80: N-((1S)-1-(4-((1,1-Dimethyl-2,3-dihydro-1H-inden-2-yl)amino)phenyl)-2,2,2-trifluoroethyl)-N-methylcyclopropanesulfonamide—Isomer 2

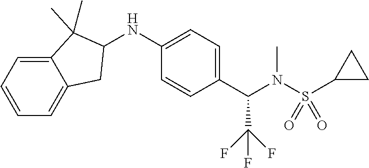 [SMILES: CN([C@@H](c1ccc(NC2Cc3ccccc3C2(C)C)cc1)C(F)(F)F)S(=O)(=O)C1CC1]

To a stirred mixture of Intermediate 14 (0.12 g, 0.29 mmol) in pyridine (1.2 mL) was added cyclopropanesulfonyl chloride (0.24 g, 1.71 mmol) and stirred at 70° C. for 6 h. The mixture was diluted with water and extracted with EtOAc. The organic layer was dried over Na₂SO₄, filtered and concentrated under reduced pressure. Purified by preparative SFC (Chiralpak®-AD-H, 30×250 mm×5 μm, temp 30° C., Back Pressure 100 bar, flow rate: 100 g/min, 75% CO₂ with 25% MeOH modifier) and a 2^{nd} time (Chiralcel-OX-H, 30×250 mm×5 μm, temp 30° C., Back Pressure 100 bar, flow rate: 100 g/min, 80% CO₂ with 20% MeOH modifier) to provide Example 79 (15 mg) as Peak 1 and Example 80 (15 mg) as Peak 2. Example 79: ¹H NMR (400 MHz; DMSO-d₆) δ: 7.22-7.12 (m, 6H), 6.80 (d, 2H), 5.99 (d, 1H), 5.50 (dd, 1H), 4.00 (dd, 1H), 3.18 (dd, 1H), 2.77-2.70 (m, 4H), 2.65-2.58 (m, 1H), 1.32 (s, 3H), 1.09 (s, 3H), 1.08-0.92 (m, 4H). LCMS (Method A): 453.5 [M+H]⁺. Example 80: ¹H NMR (400 MHz; DMSO-d₆) δ: 7.22-7.12 (m, 6H), 6.80 (d, 2H), 6.01 (d, 1H), 5.50 (dd, 1H), 4.01 (dd, 1H), 3.18 (dd, 1H), 2.77-2.70 (m, 4H), 2.65-2.58 (m, 1H), 1.32 (s, 3H), 1.09 (s, 3H), 1.08-0.92 (m, 4H). LCMS (Method A): 453.5 [M+H]⁺.

### Example 81: N-((1S)-1-(4-((1,1-Dimethyl-2,3-dihydro-1H-inden-2-yl)amino)phenyl)-2,2,2-trifluoroethyl)tetrahydro-2H-thiopyran-4-carboxamide 1,1-dioxide—Isomer 1

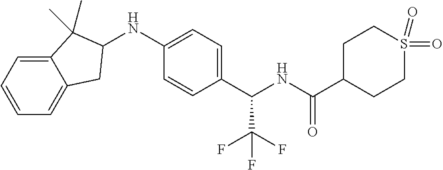 [SMILES: CC1(C)c2ccccc2CC1Nc1ccc([C@H](NC(=O)C2CCS(=O)(=O)CC2)C(F)(F)F)cc1]

### Example 82: N-((1S)-1-(4-((1,1-Dimethyl-2,3-dihydro-1H-inden-2-yl)amino)phenyl)-2,2,2-trifluoroethyl)tetrahydro-2H-thiopyran-4-carboxamide 1,1-dioxide—Isomer 2

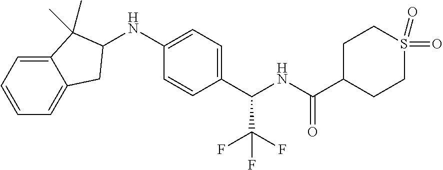 [SMILES: CC1(C)c2ccccc2CC1Nc1ccc([C@H](NC(=O)C2CCS(=O)(=O)CC2)C(F)(F)F)cc1]

The title compounds were prepared in an analogous manner to Intermediate 11 from Intermediate 51 (1.90 g, 4.58 mmol), 1,1-dimethyl-2,3-dihydro-1H-inden-2-amine hydrochloride (0.72 g, 3.67 mmol, CAS 74413-86-8), K₂CO₃ (1.9 g, 13.8 mmol), BrettphosPdG1 (0.36 g, 0.46 mmol) and BrettPhos (0.24 g, 0.46 mmol) in tBuOH (57 mL) at 160° C. for 1 h. Purified by preparative HPLC (X-Select C18, 19×250 mm×5 μm, flow rate: 18 mL/min, 10 mM aqueous NH₄HCO₃ with MeCN 40% to 65% over 6 min, held at 65% for 5 min, ramped to 98% over 0.6 min and held for 5.4 min). Chiral purification was carried out by preparative SFC (Chiralpak®-OD-H, 30×250 mm×5 μm, temp 30° C., Back Pressure 100 bar, flow rate: 100 g/min, 80% CO₂ with 20% MeOH containing 0.2% diethylamine modifier) to provide Example 81 (34 mg) as Peak 1 and Example 82 (36 mg) as Peak 2. Example 81: ¹H NMR (400 MHz; DMSO-d₆) δ: 9.03 (d, 1H), 7.23-7.12 (m, 6H), 6.74 (d, 2H), 5.83 (d, 1H), 5.52-5.48 (m, 1H), 3.99 (dd, 1H), 3.21-3.08 (m, 5H), 2.75-2.61 (m, 2H), 2.12-1.95 (m, 4H), 1.31 (s, 3H), 1.08 (s, 3H). LCMS (Method A): 495.5 [M+H]⁺. Example 82: ¹H NMR (400 MHz; DMSO-d₆) δ: 9.03 (d, 1H), 7.23-7.12 (m, 6H), 6.74 (d, 2H), 5.83 (d, 1H), 5.52-5.48 (m, 1H), 3.99 (dd, 1H), 3.21-3.08 (m, 5H), 2.75-2.61 (m, 2H), 2.12-1.95 (m, 4H), 1.31 (s, 3H), 1.08 (s, 3H). LCMS (Method A): 495.5 [M+H]⁺.

### Example 83: N-(1-(5-((1,1-Dimethyl-2,3-dihydro-1H-inden-2-yl)amino)pyridin-2-yl)-2,2,2-trifluoroethyl)-N-methylazetidine-3-carboxamide—Isomer 1

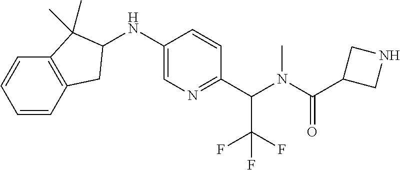 [SMILES: CN(C(=O)C1CNC1)C(c1ccc(NC2Cc3ccccc3C2(C)C)cn1)C(F)(F)F]

### Example 84: N-(1-(5-((1,1-Dimethyl-2,3-dihydro-1H-inden-2-yl)amino)pyridin-2-yl)-2,2,2-trifluoroethyl)-N-methylazetidine-3-carboxamide—Isomer 2

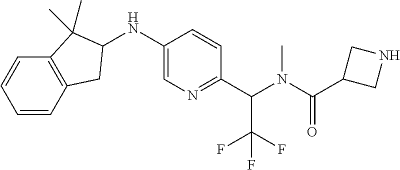 [SMILES: CN(C(=O)C1CNC1)C(c1ccc(NC2Cc3ccccc3C2(C)C)cn1)C(F)(F)F]

### Example 85: N-(1-(5-((1,1-Dimethyl-2,3-dihydro-1H-inden-2-yl)amino)pyridin-2-yl)-2,2,2-trifluoroethyl)-N-methylazetidine-3-carboxamide—Isomer 3

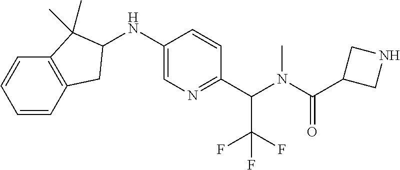 [SMILES: CN(C(=O)C1CNC1)C(c1ccc(NC2Cc3ccccc3C2(C)C)cn1)C(F)(F)F]

### Example 86: N-(1-(5-((1,1-Dimethyl-2,3-dihydro-1H-inden-2-yl)amino)pyridin-2-yl)-2,2,2-trifluoroethyl)-N-methylazetidine-3-carboxamide—Isomer 4

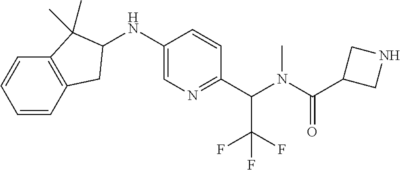 [SMILES: CN(C(=O)C1CNC1)C(c1ccc(NC2Cc3ccccc3C2(C)C)cn1)C(F)(F)F]

Title compounds were prepared from Intermediate 69 (100 mg) by preparative SFC (Chiralpak®-IG-H, 30×250 mm×5 μm, temp 30° C., Back Pressure 100 bar, flow rate: 100 g/min, 50% CO₂ with 50% (10 mM NH₄HCO₃ in MeOH) modifier) and a 2^{nd} time (Chiralcel-OX-H, 46×250 mm×5 μm, temp 30° C., Back Pressure 100 bar, flow rate: 100 g/min, 60% CO₂ with 40% (10 mM NH₄HCO₃ in MeOH) modifier) to provide Example 83 (17 mg) as Peak 1, Example 84 (18 mg) as Peak 2, Example 85 (19 mg) as Peak 3 and Example 86 (16 mg) as Peak 4. Example 83: ¹H NMR (400 MHz; DMSO-d₆) δ: 8.14-8.12 (m, 1H), 7.22-7.09 (m, 6H), 6.35 (dd, 1H), 6.20 (d, 1H), 4.08-3.99 (m, 1H), 3.86-3.70 (m, 1H), 3.67 (dd, 2H), 3.55 (t, 1H), 3.47 (t, 1H), 3.21 (dd, 1H), 2.78-2.70 (m, 4H), 1.31 (s, 3H), 1.10 (s, 3H)—NH not observed. LCMS (Method A): 433.5 [M+H]⁺. Example 84: ¹H NMR (400 MHz; DMSO-d₆) δ: 8.14-8.12 (m, 1H), 7.22-7.09 (m, 6H), 6.35 (dd, 1H), 6.20 (d, 1H), 4.04 (dd, 1H), 3.86-3.63 (m, 3H), 3.55 (t, 1H), 3.47 (t, 1H), 3.19 (dd, 1H), 2.78-2.70 (m, 4H), 1.32 (s, 3H), 1.10 (s, 3H)—NH not observed. LCMS (Method A): 433.5 [M+H]⁺. Example 85: ¹H NMR (400 MHz; DMSO-de) δ: 8.14-8.12 (m, 1H), 7.22-7.09 (m, 6H), 6.35 (dd, 1H), 6.20 (d, 1H), 4.04 (dd, 1H), 3.86-3.70 (m, 1H), 3.67 (dd, 2H), 3.55 (t, 1H), 3.47 (t, 1H), 3.19 (dd, 1H), 2.78-2.70 (m, 4H), 1.31 (s, 3H), 1.10 (s, 3H)—NH not observed. LCMS (Method A): 433.5 [M+H]⁺. Example 86: ¹H NMR (400 MHz; DMSO-d₆) δ: 8.14-8.12 (m, 1H), 7.22-7.09 (m, 6H), 6.35 (dd, 1H), 6.20 (d, 1H), 4.04 (dd, 1H), 3.86-3.63 (m, 3H), 3.55 (t, 1H), 3.47 (t, 1H), 3.19 (dd, 1H), 2.78-2.70 (m, 4H), 1.32 (s, 3H), 1.10 (s, 3H)—NH not observed. LCMS (Method A): 433.5 [M+H]⁺.

### Example 87: N-(1-(6-((1,1-Dimethyl-2,3-dihydro-1H-inden-2-yl)amino)pyridin-3-yl)-2,2,2-trifluoroethyl)-N-methylazetidine-3-carboxamide—Isomer 1

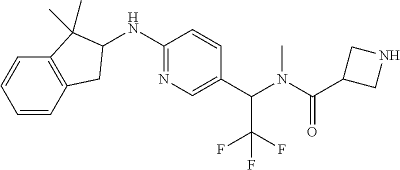 [SMILES: CN(C(=O)C1CNC1)C(c1ccc(NC2Cc3ccccc3C2(C)C)nc1)C(F)(F)F]

### Example 88: N-(1-(6-((1,1-Dimethyl-2,3-dihydro-1H-inden-2-yl)amino)pyridin-3-yl)-2,2,2-trifluoroethyl)-N-methylazetidine-3-carboxamide—Isomer 2

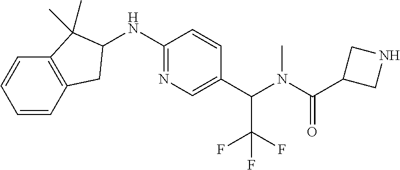 [SMILES: CN(C(=O)C1CNC1)C(c1ccc(NC2Cc3ccccc3C2(C)C)nc1)C(F)(F)F]

### Example 89: N-(1-(6-((1,1-Dimethyl-2,3-dihydro-1H-inden-2-yl)amino)pyridin-3-yl)-2,2,2-trifluoroethyl)-N-methylazetidine-3-carboxamide—Isomer 3

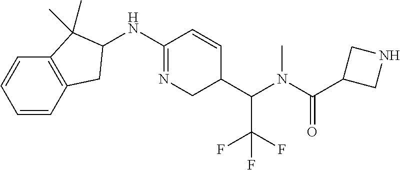 [SMILES: CN(C(=O)C1CNC1)C(C1C=CC(NC2Cc3ccccc3C2(C)C)=NC1)C(F)(F)F]

Title compounds were prepared from Intermediate 70 (100 mg) by preparative SFC (Chiralcel-OX-H, 30×250 mm×5 μm, temp 30° C., Back Pressure 120 bar, flow rate: 100 g/min, CO₂ with 45% (10 mM NH₄HCO₃ in MeOH) modifier over 7 min then to 60% (10 mM NH₄HCO₃ in MeOH) modifier for 10 min) to provide Example 87 (15 mg) as Peak 1, Example 88 (4.6 mg) as Peak 2 and Example 89 (8.7 mg) as Peak 3. Example 87: H NMR (400 MHz; DMSO-d₆) δ: 7.97-7.94 (m, 1H), 7.39 (d, 1H), 7.22-7.11 (m, 4H), 6.91 (d, 1H), 6.67 (d, 1H), 6.34 (dd, 1H), 4.59 (dd, 1H), 3.88-3.70 (m, 1H), 3.66 (dd, 2H), 3.55 (t, 1H), 3.48 (t, 1H), 3.15 (dd, 1H), 2.79-2.68 (m, 4H), 1.28 (s, 3H), 1.08 (s, 3H)—NH not observed. LCMS (Method G): 433.6 [M+H]⁺. Example 88: ¹H NMR (400 MHz; DMSO-d₆) δ: 7.97-7.94 (m, 1H), 7.39 (d, 1H), 7.22-7.11 (m, 4H), 6.91 (d, 1H), 6.67 (d, 1H), 6.34 (dd, 1H), 4.59 (dd, 1H), 3.87-3.63 (m, 3H), 3.57-3.45 (m, 2H), 3.15 (dd, 1H), 2.79-2.68 (m, 4H), 1.28 (s, 3H), 1.08 (s, 3H)—NH not observed. LCMS (Method G): 433.5 [M+H]⁺. Example 89: ¹H NMR (400 MHz; DMSO-d₆) δ: 7.97-7.94 (m, 1H), 7.39 (d, 1H), 7.22-7.11 (m, 4H), 6.91 (d, 1H), 6.67 (d, 1H), 6.34 (dd, 1H), 4.59 (dd, 1H), 3.88-3.70 (m, 1H), 3.66 (dd, 2H), 3.55 (t, 1H), 3.48 (t, 1H), 3.15 (dd, 1H), 2.79-2.68 (m, 4H), 1.29 (s, 3H), 1.08 (s, 3H)—NH not observed. LCMS (Method H): 433.6 [M+H]⁺.

### Example 90: N-(1-(5-((1,1-Dimethyl-2,3-dihydro-1H-inden-2-yl)amino)pyridin-2-yl)-2,2,2-trifluoroethyl)-N,1-dimethylazetidine-3-carboxamide—Isomer 1

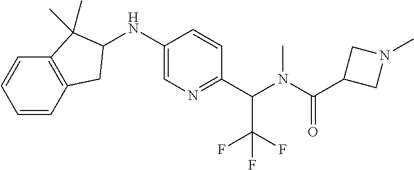 [SMILES: CN1CC(C(=O)N(C)C(c2ccc(NC3Cc4ccccc4C3(C)C)cn2)C(F)(F)F)C1]

### Example 91: N-(1-(5-((1,1-Dimethyl-2,3-dihydro-1H-inden-2-yl)amino)pyridin-2-yl)-2,2,2-trifluoroethyl)-N,1-dimethylazetidine-3-carboxamide—Isomer 2

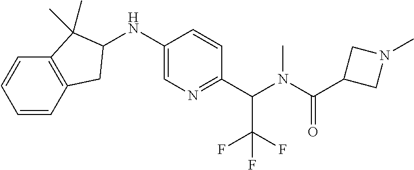 [SMILES: CN1CC(C(=O)N(C)C(c2ccc(NC3Cc4ccccc4C3(C)C)cn2)C(F)(F)F)C1]

### Example 92: N-(1-(5-((1,1-Dimethyl-2,3-dihydro-1H-inden-2-yl)amino)pyridin-2-yl)-2,2,2-trifluoroethyl)-N,1-dimethylazetidine-3-carboxamide—Isomer 3

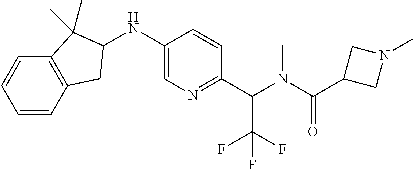 [SMILES: CN1CC(C(=O)N(C)C(c2ccc(NC3Cc4ccccc4C3(C)C)cn2)C(F)(F)F)C1]

To a stirred mixture of Intermediate 69 (0.30 g, 0.69 mmol) in MeOH (12 mL) was added AcOH (1 drop, catalytic) and paraformaldehyde (41 mg, 1.38 mmol) and stirred for 16 h at 60° C. Mixture was cooled then sodium triacetoxyborohydride (0.13 g, 2.08 mmol) was added at 0° C. then stirred at RT for 2 h. The mixture was diluted with water and extracted with EtOAc. The organic layer was dried over Na₂SO₄, filtered and concentrated under reduced pressure. Purified by preparative HPLC (Gemini-NX, 21.5×250 mm×5 μm, flow rate: 18 mL/min, 10 mM aqueous NH₄HCO₃ with MeCN 40% to 85% over 9 min, held at 85% for 4.5 min, ramped to 98% over 0.1 min and held for 4.4 min). Chiral purification was carried out by preparative SFC (LUX Cellulose-2, 30×250 mm×5 μm, temp 30° C., Back Pressure 100 bar, flow rate: 100 g/min, 73% CO₂ with 27% (10 mM NH₄HCO₃ in methanol/MeCN 1:1) modifier followed by (Chiralpak®-AD-H, 30×250 mm×5 μm, temp 30° C., Back Pressure 100 bar, flow rate: 100 g/min, 73% CO₂ with 27% (10 mM NH₄HCO₃ in methanol/MeCN 1:1) modifier to provide Example 90 (8.9 mg) as Peak 1 and Example 91 (3.3 mg) as Peak 3 and Example 92 (7.9 mg) as Peak 4. Example 90: ¹H NMR (400 MHz; DMSO-d₆) δ: 8.15-8.12 (m, 1H), 7.22-7.07 (m, 6H), 6.33 (dd, 1H), 6.20 (d, 1H), 4.04 (dd, 1H), 3.58-3.40 (m, 3H), 3.19 (dd, 1H), 3.10-3.02 (m, 2H), 2.81 (s, 3H), 2.78-2.70 (m, 1H), 2.17 (s, 3H), 1.32 (s, 3H), 1.10 (s, 3H). LCMS (Method A): 447.4 [M+H]⁺. Example 91: ¹H NMR (400 MHz; DMSO-d₆) δ: 8.15-8.12 (m, 1H), 7.22-7.07 (m, 6H), 6.32 (dd, 1H), 6.21 (d, 1H), 4.04 (dd, 1H), 3.58-3.40 (m, 3H), 3.19 (dd, 1H), 3.09-3.04 (m, 2H), 2.80 (s, 3H), 2.78-2.70 (m, 1H), 2.17 (s, 3H), 1.31 (s, 3H), 1.10 (s, 3H). LCMS (Method A): 447.4 [M+H]⁺. Example 92: ¹H NMR (400 MHz; DMSO-d₆) δ: 8.15-8.12 (m, 1H), 7.22-7.07 (m, 6H), 6.33 (dd, 1H), 6.21 (d, 1H), 4.04 (dd, 1H), 3.58-3.40 (m, 3H), 3.19 (dd, 1H), 3.09-3.04 (m, 2H), 2.80 (s, 3H), 2.78-2.70 (m, 1H), 2.17 (s, 3H), 1.32 (s, 3H), 1.10 (s, 3H). LCMS (Method A): 447.4 [M+H]⁺.

### Example 93: N-(1-(6-((1,1-Dimethyl-2,3-dihydro-1H-inden-2-yl)amino)pyridin-3-yl)-2,2,2-trifluoroethyl)-N,1-dimethylazetidine-3-carboxamide—Isomer 1

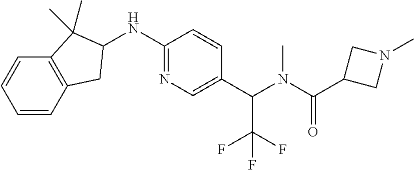 [SMILES: CN1CC(C(=O)N(C)C(c2ccc(NC3Cc4ccccc4C3(C)C)nc2)C(F)(F)F)C1]

### Example 94: N-(1-(6-((1,1-Dimethyl-2,3-dihydro-1H-inden-2-yl)amino)pyridin-3-yl)-2,2,2-trifluoroethyl)-N,1-dimethylazetidine-3-carboxamide—Isomer 2

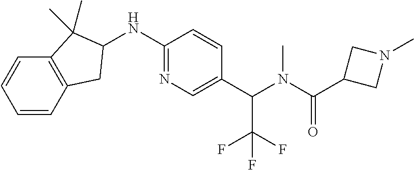 [SMILES: CN1CC(C(=O)N(C)C(c2ccc(NC3Cc4ccccc4C3(C)C)nc2)C(F)(F)F)C1]

### Example 95: N-(1-(6-((1,1-Dimethyl-2,3-dihydro-1H-inden-2-yl)amino)pyridin-3-yl)-2,2,2-trifluoroethyl)-N,1-dimethylazetidine-3-carboxamide—Isomer 3

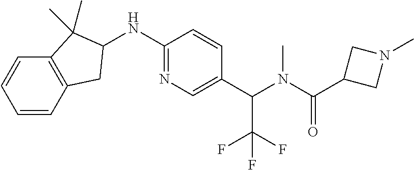 [SMILES: CN1CC(C(=O)N(C)C(c2ccc(NC3Cc4ccccc4C3(C)C)nc2)C(F)(F)F)C1]

To a stirred mixture of Intermediate 70 (0.20 g, 0.46 mmol) in MeOH (8 mL) was added AcOH (1 drop, catalytic) and paraformaldehyde (30 mg, 0.92 mmol) and stirred for 16 h at RT. Sodium cyanoborohydride (0.13 g, 2.08 mmol) was added at 0° C. then stirred at RT for 2 h. The mixture was diluted with water and extracted with EtOAc. The organic layer was dried over Na₂SO₄, filtered and concentrated under reduced pressure. Purified by preparative HPLC (Gemini-NX, 21.5×250 mm×5 μm, flow rate: 18 mL/min, 10 mM aqueous NH₄HCO₃ with MeCN 35% to 70% over 6 min, held at 70% for 6 min, ramped to 98% over 0.1 min and held for 2.9 min). Chiral purification was carried out by preparative SFC (Chiralpak®-IG, 30×250 mm×5 μm, temp 30° C., Back Pressure 100 bar, flow rate: 100 g/min, 67% CO₂ with 33% (10 mM NH₄HCO₃ in methanol/MeCN 1:1) modifier to provide Example 93 (7.7 mg) as Peak 1 and Example 94 (7.2 mg) as Peak 2 and Example 95 (6.3 mg) as Peak 3. Example 93: ¹H NMR (400 MHz; DMSO-d₆) δ: 7.98-7.93 (m, 1H), 7.40-7.36 (m, 1H), 7.21-7.09 (m, 4H), 6.91 (d, 1H), 6.66 (d, 1H), 6.32 (dd, 1H), 4.58 (dd, 1H), 3.58-3.38 (m, 3H), 3.15 (dd, 1H), 3.06 (dd, 2H), 2.80-2.68 (m, 4H), 2.17 (s, 3H), 1.28 (s, 3H), 1.08 (s, 3H). LCMS (Method H): 447.7 [M+H]⁺. Example 94: ¹H NMR (400 MHz; DMSO-d₆) δ: 7.98-7.93 (m, 1H), 7.41-7.36 (m, 1H), 7.21-7.11 (m, 4H), 6.91 (d, 1H), 6.67 (d, 1H), 6.32 (dd, 1H), 4.57 (dd, 1H), 3.58-3.38 (m, 3H), 3.16 (dd, 1H), 3.07 (dd, 2H), 2.80-2.68 (m, 4H), 2.17 (s, 3H), 1.29 (s, 3H), 1.08 (s, 3H). LCMS (Method H): 447.7 [M+H]⁺. Example 95: ¹H NMR (400 MHz; DMSO-d₆) δ: 7.98-7.93 (m, 1H), 7.41-7.36 (m, 1H), 7.21-7.11 (m, 4H), 6.91 (d, 1H), 6.67 (d, 1H), 6.32 (dd, 1H), 4.58 (dd, 1H), 3.58-3.38 (m, 3H), 3.15 (dd, 1H), 3.06 (dd, 2H), 2.80-2.68 (m, 4H), 2.17 (s, 3H), 1.29 (s, 3H), 1.08 (s, 3H). LCMS (Method H): 447.6 [M+H]⁺.

### Example 96: N-(1-(5-((1,1-Dimethyl-2,3-dihydro-1H-inden-2-yl)amino)pyridin-2-yl)-2,2,2-trifluoroethyl)-N-methyl-2-oxo-1,2-dihydropyridine-4-carboxamide—Isomer 1

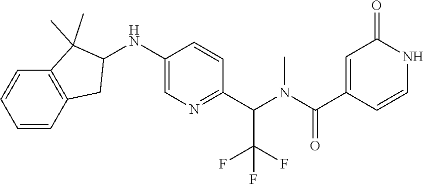 [SMILES: CN(C(=O)c1cc[nH]c(=O)c1)C(c1ccc(NC2Cc3ccccc3C2(C)C)cn1)C(F)(F)F]

### Example 97: N-(1-(5-((1,1-Dimethyl-2,3-dihydro-1H-inden-2-yl)amino)pyridin-2-yl)-2,2,2-trifluoroethyl)-N-methyl-2-oxo-1,2-dihydropyridine-4-carboxamide—Isomer 2

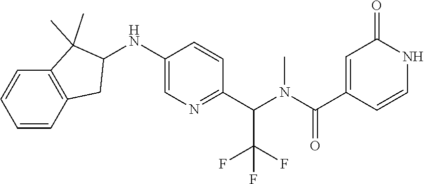 [SMILES: CN(C(=O)c1cc[nH]c(=O)c1)C(c1ccc(NC2Cc3ccccc3C2(C)C)cn1)C(F)(F)F]

### Example 98: N-(1-(5-((1,1-Dimethyl-2,3-dihydro-1H-inden-2-yl)amino)pyridin-2-yl)-2,2,2-trifluoroethyl)-N-methyl-2-oxo-1,2-dihydropyridine-4-carboxamide—Isomer 3

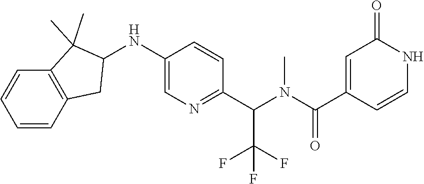 [SMILES: CN(C(=O)c1cc[nH]c(=O)c1)C(c1ccc(NC2Cc3ccccc3C2(C)C)cn1)C(F)(F)F]

### Example 99: N-(1-(5-((1,1-Dimethyl-2,3-dihydro-1H-inden-2-yl)amino)pyridin-2-yl)-2,2,2-trifluoroethyl)-N-methyl-2-oxo-1,2-dihydropyridine-4-carboxamide—Isomer 4

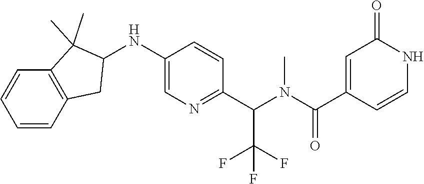 [SMILES: CN(C(=O)c1cc[nH]c(=O)c1)C(c1ccc(NC2Cc3ccccc3C2(C)C)cn1)C(F)(F)F]

The title compounds were prepared in an analogous manner to Intermediate 3 from Intermediate 57 (0.35 g, 0.91 mmol), pyridine (3.5 mL), T3P® (50% EtOAc, 2.80 mL, 4.54 mmol) and 2-oxo-1,2-dihydropyridine-4-carboxylic acid (0.19 g, 1.36 mmol, CAS 22282-72-0) in THF (5 mL) at 60° C. for 16 h. Purified by column chromatography (silica gel, eluting 30% EtOAc in petroleum ether). Further purification was carried out by achiral preparative SFC (YMC-Pack-Diol, 30×250 mm×5 μm, temp 30° C., Back Pressure 100 bar, flow rate: 100 g/min, 75% CO₂ with 25% MeOH modifier). Chiral purification was carried out by preparative SFC (Chiralpak®-IG, 30×250 mm×5 μm, temp 30° C., Back Pressure 100 bar, flow rate: 100 g/min, 60% CO₂ with 40% (10 mM NH₄HCO₃ in methanol/MeCN 1:1) modifier to provide Example 96 (35 mg) as Peak 1, Example 97 (27 mg) as Peak 2, Example 98 (31 mg) as Peak 3 and Example 99 (35 mg) as Peak 4. Example 96: ¹H NMR (400 MHz; DMSO-d₆) δ: 11.66 (br s, 1H), 8.17 (s, 1H), 7.52-7.45 (m, 1H), 7.23-7.12 (m, 6H), 6.37 (dd, 1H), 6.28 (d, 2H), 6.14-6.07 (m, 1H), 4.06 (dd, 1H), 3.19 (dd, 1H), 2.90-2.84 (m, 3H), 2.75 (dd, 1H), 1.33 (s, 3H), 1.10 (s, 3H). LCMS (Method A): 471.2 [M+H]⁺. Example 97: ¹H NMR (400 MHz; DMSO-d₆) δ: 11.74 (br s, 1H), 8.17 (s, 1H), 7.52-7.46 (m, 1H), 7.23-7.12 (m, 6H), 6.38 (dd, 1H), 6.32-6.22 (m, 2H), 6.14-6.07 (m, 1H), 4.06 (dd, 1H), 3.20 (dd, 1H), 2.88 (s, 3H), 2.75 (dd, 1H), 1.32 (s, 3H), 1.10 (s, 3H). LCMS (Method A): 471.2 [M+H]⁺. Example 98: ¹H NMR (400 MHz; DMSO-d₆) δ: 11.70 (br s, 1H), 8.17 (s, 1H), 7.53-7.48 (m, 1H), 7.23-7.12 (m, 6H), 6.38 (dd, 1H), 6.32-6.18 (m, 2H), 6.08-6.04 (m, 1H), 4.06 (dd, 1H), 3.20 (dd, 1H), 2.87 (s, 3H), 2.75 (dd, 1H), 1.32 (s, 3H), 1.11 (s, 3H). LCMS (Method A): 471.2 [M+H]⁺. Example 99: ¹H NMR (400 MHz; DMSO-d₆) δ: 10.50 (br s, 1H), 8.17 (s, 1H), 7.53-7.48 (m, 1H), 7.23-7.12 (m, 6H), 6.38 (dd, 1H), 6.23-6.18 (m, 2H), 6.08-6.04 (m, 1H), 4.06 (dd, 1H), 3.19 (dd, 1H), 2.90-2.85 (m, 3H), 2.75 (dd, 1H), 1.32 (s, 3H), 1.10 (s, 3H). LCMS (Method A): 471.2 [M+H]⁺.

### Example 100: N-(1-(6-((1,1-Dimethyl-2,3-dihydro-1H-inden-2-yl)amino)pyridin-3-yl)-2,2,2-trifluoroethyl)-N-methyl-2-oxo-1,2-dihydropyridine-4-carboxamide—Isomer 1

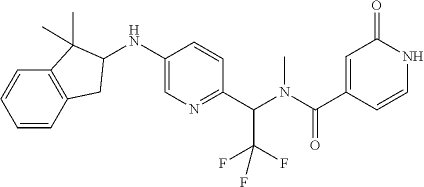 [SMILES: CN(C(=O)c1cc[nH]c(=O)c1)C(c1ccc(NC2Cc3ccccc3C2(C)C)cn1)C(F)(F)F]

### Example 101: N-(1-(6-((1,1-Dimethyl-2,3-dihydro-1H-inden-2-yl)amino)pyridin-3-yl)-2,2,2-trifluoroethyl)-N-methyl-2-oxo-1,2-dihydropyridine-4-carboxamide—Isomer 2

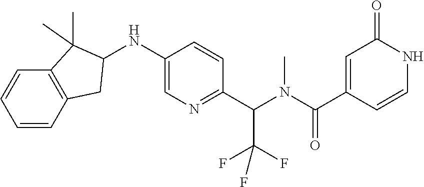 [SMILES: CN(C(=O)c1cc[nH]c(=O)c1)C(c1ccc(NC2Cc3ccccc3C2(C)C)cn1)C(F)(F)F]

### Example 102: N-(1-(6-((1,1-Dimethyl-2,3-dihydro-1H-inden-2-yl)amino)pyridin-3-yl)-2,2,2-trifluoroethyl)-N-methyl-2-oxo-1,2-dihydropyridine-4-carboxamide—Isomer 3

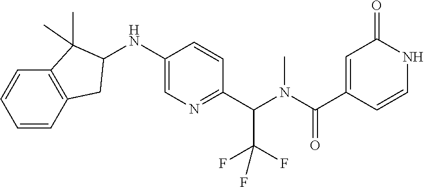 [SMILES: CN(C(=O)c1cc[nH]c(=O)c1)C(c1ccc(NC2Cc3ccccc3C2(C)C)cn1)C(F)(F)F]

### Example 103: N-(1-(6-((1,1-Dimethyl-2,3-dihydro-1H-inden-2-yl)amino)pyridin-3-yl)-2,2,2-trifluoroethyl)-N-methyl-2-oxo-1,2-dihydropyridine-4-carboxamide—Isomer 4

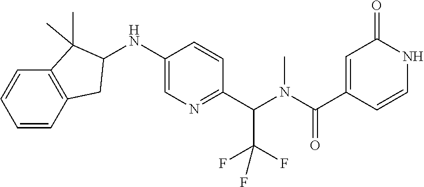 [SMILES: CN(C(=O)c1cc[nH]c(=O)c1)C(c1ccc(NC2Cc3ccccc3C2(C)C)cn1)C(F)(F)F]

To a stirred solution of 2-oxo-1,2-dihydropyridine-4-carboxylic acid (0.36 g, 2.58 mmol, CAS 22282-72-0) in DCM (3 mL) at 0° C. was added oxalyl chloride (0.29 mL, 3.38 mmol) then the mixture was stirred at RT for 1 h. A solution of Intermediate 26 (0.30 g, 0.88 mmol) and triethylamine (0.84 mL, 6.04 mmol) in DCM (3 mL) premixed for 15 min at RT was cooled at 0° C. then the acid chloride mixture was added and the mixture stirred at RT for 2 h. The mixture was diluted with water and extracted with DCM. The organic layer was dried over Na₂SO₄, filtered and concentrated under reduced pressure. Purified by preparative HPLC (Gemini NX-C18, 19×250 mm×5 μm, flow rate: 18 mL/min, 10 mM aqueous NH₄HCO₃ with MeCN 20% to 55% over 7 min, then to 60% over 2 min, held at 65% for 4.5 min, ramped to 98% over 0.1 min). Chiral purification was carried out by preparative SFC (Chiralpak®-OX-H, 30×250 mm×5 μm, temp 30° C., Back Pressure 100 bar, flow rate: 100 g/min, 55% CO₂ with 45% (10 mM NH₄HCO₃ in methanol) modifier for 7 min then 40% CO₂ with 60% (10 mM NH₄HCO₃ in methanol) for 10 min to provide Example 100 (7.5 mg) as Peak 1, Example 101 (8.7 mg) as Peak 2, Example 102 (8.6 mg) as Peak 3 and Example 103 (8.1 mg) as Peak 4. Example 100: ¹H NMR (400 MHz; DMSO-d₆) δ: 11.75 (br s, 1H), 8.06-7.93 (m, 1H), 7.58-7.42 (m, 2H), 7.23-7.12 (m, 4H), 6.97 (d, 1H), 6.72 (d, 1H), 6.47-6.32 (m, 1H), 6.23 (d, 1H), 6.08 (d, 1H), 4.60 (dd, 1H), 3.17 (dd, 1H), 2.90-2.72 (m, 4H), 1.30 (s, 3H), 1.09 (s, 3H). LCMS (Method A): 471.5 [M+H]⁺. Example 101: ¹H NMR (400 MHz; DMSO-d₆) δ: 11.83 (br s, 1H), 8.04-7.93 (m, 1H), 7.54-7.42 (m, 2H), 7.23-7.12 (m, 4H), 6.97 (d, 1H), 6.70 (d, 1H), 6.43-6.32 (m, 1H), 6.23 (s, 1H), 6.08 (d, 1H), 4.60 (dd, 1H), 3.16 (dd, 1H), 2.87-2.72 (m, 4H), 1.30 (s, 3H), 1.09 (s, 3H). LCMS (Method A): 471.5 [M+H]⁺. Example 102: ¹H NMR (400 MHz; DMSO-d₆) δ: 11.63 (br s, 1H), 8.04-7.93 (m, 1H), 7.54-7.42 (m, 2H), 7.23-7.12 (m, 4H), 6.97 (d, 1H), 6.69 (d, 1H), 6.43-6.32 (m, 1H), 6.23 (s, 1H), 6.08 (br d, 1H), 4.60 (dd, 1H), 3.16 (dd, 1H), 2.87-2.72 (m, 4H), 1.30 (s, 3H), 1.09 (s, 3H). LCMS (Method A): 471.5 [M+H]⁺. Example 103: ¹H NMR (400 MHz; DMSO-d₆) δ: 11.33 (br s, 1H), 8.04-7.93 (m, 1H), 7.54-7.42 (m, 2H), 7.23-7.12 (m, 4H), 6.97 (d, 1H), 6.69 (d, 1H), 6.43-6.32 (m, 1H), 6.23 (s, 1H), 6.08 (br d, 1H), 4.60 (dd, 1H), 3.17 (dd, 1H), 2.87-2.72 (m, 4H), 1.29 (s, 3H), 1.09 (s, 3H). LCMS (Method A): 471.5 [M+H]⁺.

### Example 104: N-((1S)-1-(5-((1,1-Dimethyl-2,3-dihydro-1H-inden-2-yl)amino)pyridin-2-yl)-2,2,2-trifluoroethyl)-N-methyl-6-oxopiperidine-3-carboxamide—Isomer 1

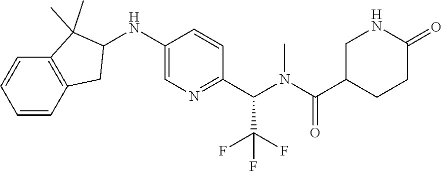 [SMILES: CN(C(=O)C1CCC(=O)NC1)[C@@H](c1ccc(NC2Cc3ccccc3C2(C)C)cn1)C(F)(F)F]

### Example 105: N-((1S)-1-(5-((1,1-Dimethyl-2,3-dihydro-1H-inden-2-yl)amino)pyridin-2-yl)-2,2,2-trifluoroethyl)-N-methyl-6-oxopiperidine-3-carboxamide—Isomer 2

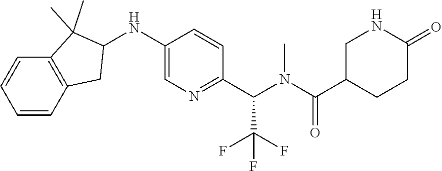 [SMILES: CN(C(=O)C1CCC(=O)NC1)[C@@H](c1ccc(NC2Cc3ccccc3C2(C)C)cn1)C(F)(F)F]

### Example 106: N-((1S)-1-(5-((1,1-Dimethyl-2,3-dihydro-1H-inden-2-yl)amino)pyridin-2-yl)-2,2,2-trifluoroethyl)-N-methyl-6-oxopiperidine-3-carboxamide—Isomer 3

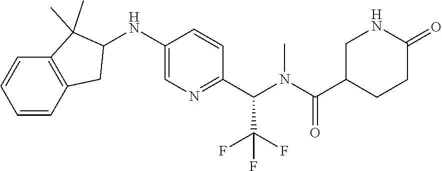 [SMILES: CN(C(=O)C1CCC(=O)NC1)[C@@H](c1ccc(NC2Cc3ccccc3C2(C)C)cn1)C(F)(F)F]

### Example 107: N-((1S)-1-(5-((1,1-Dimethyl-2,3-dihydro-1H-inden-2-yl)amino)pyridin-2-yl)-2,2,2-trifluoroethyl)-N-methyl-6-oxopiperidine-3-carboxamide—Isomer 4

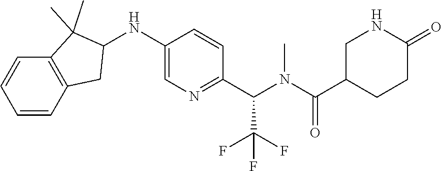 [SMILES: CN(C(=O)C1CCC(=O)NC1)[C@@H](c1ccc(NC2Cc3ccccc3C2(C)C)cn1)C(F)(F)F]

The title compounds were prepared in an analogous manner to Intermediate 3 from Intermediate 61 (0.75 g, 1.89 mmol), pyridine (3.8 mL), T3P® (50% EtOAc, 1.80 g, 5.67 mmol) and 6-oxopiperidine-3-carboxylic acid (0.32 g, 2.27 mmol, CAS 22540-50-7) at 60° C. for 16 h. Purified by preparative HPLC (XBridge C18, 19×250 mm×5 μm, flow rate: 18 mL/min, 10 mM aqueous NH₄HCO₃ with MeCN 30% to 60% over 5 min, held at 60% for 5 min, ramped to 100% over 0.1 min, held for 4.9 min). Chiral purification was carried out by preparative SFC (Chiralpak®-AS-H, 30×250 mm×5 μm, temp 30° C., Back Pressure 100 bar, flow rate: 100 g/min, 60% CO₂ with 40% MeOH modifier) then (Chiralpak®-IG, 30×250 mm×5 μm, temp 30° C., Back Pressure 100 bar, flow rate: 100 g/min, 50% CO₂ with 50% MeOH modifier) and (Chiralpak®-IG, 30×250 mm×5 μm, temp 30° C., Back Pressure 100 bar, flow rate: 100 g/min, 60% CO₂ with 40% MeOH modifier) to provide Example 104 (38 mg) as Peak 1, Example 105 (31 mg) as Peak 2, Example 106 (37 mg) as Peak 3 and Example 107 (23 mg) as Peak 4. Example 104: ¹H NMR (400 MHz; DMSO-d₆) δ: 8.17-8.14 (m, 1H), 7.48 (br s, 1H), 7.23-7.06 (m, 6H), 6.36 (dd, 1H), 6.21 (d, 1H), 4.04 (dd, 1H), 3.28-3.15 (m, 4H), 3.03 (s, 3H), 2.79-2.71 (m, 1H), 2.31-2.19 (m, 2H), 1.88-1.72 (m, 2H), 1.32 (s, 3H), 1.10 (s, 3H). LCMS (Method A): 475.5 [M+H]⁺. Example 105: ¹H NMR (400 MHz; DMSO-d₆) δ: 8.17-8.14 (m, 1H), 7.48 (br s, 1H), 7.23-7.16 (m, 6H), 6.36 (dd, 1H), 6.21 (d, 1H), 4.04 (dd, 1H), 3.28-3.15 (m, 4H), 3.03 (s, 3H), 2.79-2.71 (m, 1H), 2.31-2.19 (m, 2H), 1.88-1.72 (m, 2H), 1.32 (s, 3H), 1.10 (s, 3H). LCMS (Method A): 475.4 [M+H]⁺. Example 106: ¹H NMR (400 MHz; DMSO-d₆) δ: 8.17-8.14 (m, 1H), 7.48 (br s, 1H), 7.23-7.09 (m, 6H), 6.38 (dd, 1H), 6.21 (d, 1H), 4.05 (dd, 1H), 3.28-3.15 (m, 4H), 3.04 (s, 3H), 2.79-2.71 (m, 1H), 2.31-2.19 (m, 2H), 1.98-1.90 (m, 1H), 1.82-1.72 (m, 1H), 1.32 (s, 3H), 1.10 (s, 3H). LCMS (Method A): 475.5 [M+H]⁺. Example 107: ¹H NMR (400 MHz; DMSO-d₆) δ: 8.17-8.14 (m, 1H), 7.48 (br s, 1H), 7.23-7.08 (m, 6H), 6.38 (dd, 1H), 6.21 (d, 1H), 4.05 (dd, 1H), 3.28-3.15 (m, 4H), 3.04 (s, 3H), 2.79-2.71 (m, 1H), 2.31-2.19 (m, 2H), 1.97-1.88 (m, 1H), 1.82-1.72 (m, 1H), 1.32 (s, 3H), 1.10 (s, 3H). LCMS (Method A): 475.5 [M+H]⁺.

### Example 108: N-((1S)-1-(5-((1,1-Dimethyl-2,3-dihydro-1H-inden-2-yl)amino)pyridin-2-yl)-2,2,2-trifluoroethyl)-N-methyl-2-oxooxazolidine-5-carboxamide—Isomer 1

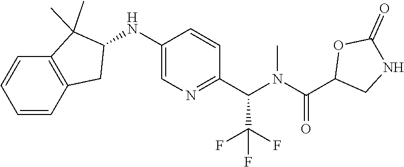 [SMILES: CN(C(=O)C1CNC(=O)O1)[C@@H](c1ccc(N[C@@H]2Cc3ccccc3C2(C)C)cn1)C(F)(F)F]

### Example 109: N-((1S)-1-(5-((1,1-Dimethyl-2,3-dihydro-1H-inden-2-yl)amino)pyridin-2-yl)-2,2,2-trifluoroethyl)-N-methyl-2-oxooxazolidine-5-carboxamide—Isomer 2

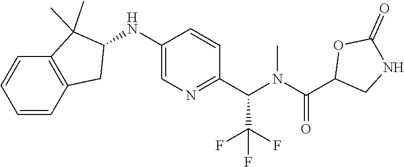 [SMILES: CN(C(=O)C1CNC(=O)O1)[C@@H](c1ccc(N[C@@H]2Cc3ccccc3C2(C)C)cn1)C(F)(F)F]

### Example 110: N-((1S)-1-(5-((1,1-Dimethyl-2,3-dihydro-1H-inden-2-yl)amino)pyridin-2-yl)-2,2,2-trifluoroethyl)-N-methyl-2-oxooxazolidine-5-carboxamide—Isomer 3

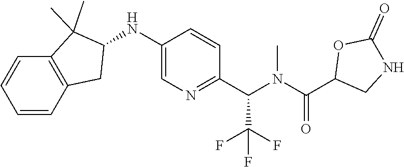 [SMILES: CN(C(=O)C1CNC(=O)O1)[C@@H](c1ccc(N[C@@H]2Cc3ccccc3C2(C)C)cn1)C(F)(F)F]

### Example 111: N-((1S)-1-(5-((1,1-Dimethyl-2,3-dihydro-1H-inden-2-yl)amino)pyridin-2-yl)-2,2,2-trifluoroethyl)-N-methyl-2-oxooxazolidine-5-carboxamide—Isomer 4

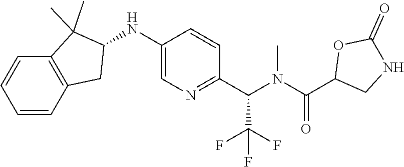 [SMILES: CN(C(=O)C1CNC(=O)O1)[C@@H](c1ccc(N[C@@H]2Cc3ccccc3C2(C)C)cn1)C(F)(F)F]

The title compounds were prepared in an analogous manner to Intermediate 3 from Intermediate 61 (0.50 g, 1.43 mmol), pyridine (2.5 mL), T3P® (50% EtOAc, 6.4 mL, 10.0 mmol) and 2-oxo-1,3-oxazolidine-5-carboxylic acid (0.28 g, 2.14 mmol, CAS 60487-08-3) at RT for 16 h. Purified by preparative HPLC (XBridge C18, 19×250 mm×5 μm, flow rate: 18 mL/min, 10 mM aqueous NH₄HCO₃ with MeCN 45% to 60% over 12 min, ramped to 98% over 0.1 min, held for 1.9 min). Chiral purification was carried out by preparative SFC (Chiralpak®-IG, 30×250 mm×5 μm, temp 30° C., Back Pressure 100 bar, flow rate: 100 g/min, 60% CO₂ with 40% MeOH modifier) to provide Example 108 (89 mg) as Peak 1, Example 109 (40 mg) as Peak 2, Example 110 (89 mg) as Peak 3 and Example 111 (54 mg) as Peak 4. Example 108: ¹H NMR (400 MHz; DMSO-d₆) δ: 8.17-8.14 (m, 1H), 7.49 (br s, 1H), 7.23-7.12 (m, 6H), 6.27 (dd, 2H), 5.55 (dd, 1H), 4.04 (dd, 1H), 3.70 (t, 1H), 3.52 (dd, 1H), 3.20 (dd, 1H), 2.97 (s, 3H), 2.74 (dd, 1H), 1.32 (s, 3H), 1.10 (s, 3H). LCMS (Method A): 463.4 [M+H]⁺. Example 109: ¹H NMR (400 MHz; DMSO-d₆) δ: 8.17-8.14 (m, 1H), 7.75 (br s, 1H), 7.23-7.12 (m, 6H), 6.27 (dd, 2H), 5.54 (dd, 1H), 4.04 (dd, 1H), 3.70 (t, 1H), 3.52 (dd, 1H), 3.20 (dd, 1H), 2.97 (s, 3H), 2.74 (dd, 1H), 1.32 (s, 3H), 1.10 (s, 3H). LCMS (Method A): 463.4 [M+H]⁺. Example 110: ¹H NMR (400 MHz; DMSO-d₆) δ: 8.16 (s, 1H), 7.77 (br s, 1H), 7.23-7.12 (m, 6H), 6.27 (dd, 2H), 5.57 (dd, 1H), 4.05 (dd, 1H), 3.78 (t, 1H), 3.49 (dd, 1H), 3.20 (dd, 1H), 2.98 (s, 3H), 2.74 (dd, 1H), 1.32 (s, 3H), 1.10 (s, 3H). LCMS (Method A): 463.4 [M+H]⁺. Example 111: ¹H NMR (400 MHz; DMSO-d₆) δ: 8.16 (s, 1H), 7.77 (br s, 1H), 7.23-7.12 (m, 6H), 6.27 (dd, 2H), 5.57 (dd, 1H), 4.05 (dd, 1H), 3.78 (t, 1H), 3.49 (dd, 1H), 3.20 (dd, 1H), 2.98 (s, 3H), 2.74 (dd, 1H), 1.32 (s, 3H), 1.10 (s, 3H). LCMS (Method A): 463.4 [M+H]⁺.

### Example 112: N-((1S)-1-(5-((1,1-Dimethyl-2,3-dihydro-1H-inden-2-yl)amino)pyridin-2-yl)-2,2,2-trifluoroethyl)-3-trans-(dimethylamino)-N-methylcyclobutane-1-carboxamide—Isomer 1

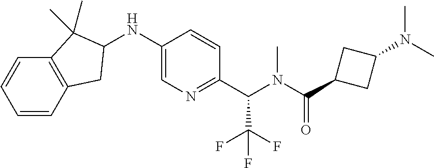 [SMILES: CN(C(=O)[C@H]1C[C@H](N(C)C)C1)[C@@H](c1ccc(NC2Cc3ccccc3C2(C)C)cn1)C(F)(F)F]

### Example 113: N-((1S)-1-(5-((1,1-Dimethyl-2,3-dihydro-1H-inden-2-yl)amino)pyridin-2-yl)-2,2,2-trifluoroethyl)-3-trans-(dimethylamino)-N-methylcyclobutane-1-carboxamide—Isomer 2

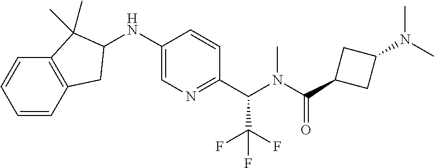 [SMILES: CN(C(=O)[C@H]1C[C@H](N(C)C)C1)[C@@H](c1ccc(NC2Cc3ccccc3C2(C)C)cn1)C(F)(F)F]

The title compounds were prepared in an analogous manner to Intermediate 3 from Intermediate 61 (0.20 g, 0.57 mmol), pyridine (2.0 mL), T3P® (50% EtOAc, 1.82 mL, 2.86 mmol) and (1,3-trans)-3-(dimethylamino)cyclobutane-1-carboxylic acid hydrochloride (0.16 g, 0.62 mmol, CAS 2230913-68-3) at RT for 16 h. Purified by preparative HPLC (Gemini-NX C18, 19×250 mm×5 μm, flow rate: 18 mL/min, 10 mM aqueous NH₄HCO₃ with MeCN 40% to 80% over 9 min, held at 80% for 3 min, ramped to 100% over 0.1 min, held for 4.9 min). Chiral purification was carried out by preparative SFC (Chiralpak®-IG, 30×250 mm×5 μm, temp 30° C., Back Pressure 100 bar, flow rate: 100 g/min, 60% CO₂ with 40% MeOH modifier) to provide Example 112 (28 mg) as Peak 1 and Example 113 (29 mg) as Peak 2. Example 112: ¹H NMR (400 MHz; DMSO-d₆) δ: 8.15-8.13 (m, 1H), 7.23-7.08 (m, 6H), 6.38 (dd, 1H), 6.19 (dd, 1H), 4.02 (dd, 1H), 3.29-3.22 (m, 1H), 3.19 (dd, 1H), 2.83 (s, 3H), 2.78-2.60 (m, 2H), 2.30-2.05 (m, 4H), 2.00 (s, 6H), 1.32 (s, 3H), 1.10 (s, 3H). LCMS (Method F): 475.9 [M+H]⁺. Example 113: ¹H NMR (400 MHz; DMSO-d₆) δ: 8.15-8.13 (m, 1H), 7.23-7.08 (m, 6H), 6.38 (dd, 1H), 6.19 (dd, 1H), 4.02 (dd, 1H), 3.29-3.22 (m, 1H), 3.19 (dd, 1H), 2.82 (s, 3H), 2.78-2.58 (m, 2H), 2.30-2.05 (m, 4H), 2.00 (s, 6H), 1.32 (s, 3H), 1.10 (s, 3H). LCMS (Method F): 475.9 [M+H]⁺.

### Example 114: N-((1S)-1-(6-((1,1-Dimethyl-2,3-dihydro-1H-inden-2-yl)amino)pyridin-3-yl)-2,2,2-trifluoroethyl)-3-trans-(dimethylamino)-N-methylcyclobutane-1-carboxamide—Isomer 1

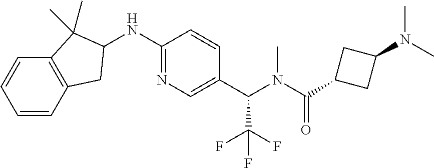 [SMILES: CN(C(=O)[C@H]1C[C@H](N(C)C)C1)[C@@H](c1ccc(NC2Cc3ccccc3C2(C)C)nc1)C(F)(F)F]

### Example 115: N-((1S)-1-(6-((1,1-Dimethyl-2,3-dihydro-1H-inden-2-yl)amino)pyridin-3-yl)-2,2,2-trifluoroethyl)-3-trans-(dimethylamino)-N-methylcyclobutane-1-carboxamide—Isomer 2

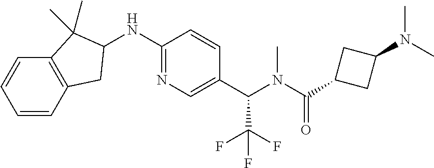 [SMILES: CN(C(=O)[C@H]1C[C@H](N(C)C)C1)[C@@H](c1ccc(NC2Cc3ccccc3C2(C)C)nc1)C(F)(F)F]

The title compounds were prepared in an analogous manner to Intermediate 3 from Intermediate 64 (0.35 g, 0.90 mmol), pyridine (1.8 mL), T3P® (50% EtOAc, 3.50 mL, 5.50 mmol) and (1,3-trans)-3-(dimethylamino)cyclobutane-1-carboxylic acid hydrochloride (0.26 g, 1.81 mmol, CAS 2230913-68-3) at RT for 16 h. Purified by preparative HPLC (XBridge C18,19×250 mm×5 μm, flow rate: 19 mL/min, 10 mM aqueous NH₄HCO₃ with MeCN 35% to 65% over 6 min, held at 65% for 5.9 min, ramped to 100% over 0.1 min, held for 5 min). Chiral purification was carried out by preparative SFC (Chiralpak®-AD-H, 30×250 mm×5 μm, temp 30° C., Back Pressure 100 bar, flow rate: 100 g/min, 70% CO₂ with 30% (5 mM NH₄HCO₃ in MeOH) modifier) to provide Example 114 (40 mg) as Peak 1 and Example 115 (31 mg) as Peak 2. Example 114: ¹H NMR (400 MHz; DMSO-d₆) δ: 7.94 (s, 1H), 7.42-7.34 (m, 1H), 7.22-7.10 (m, 4H), 6.91 (d, 1H), 6.67 (d, 1H), 6.34 (dd, 1H), 4.58 (dd, 1H), 3.29-3.23 (m, 1H), 3.15 (dd, 1H), 2.80-2.72 (m, 4H), 2.68-2.60 (m, 1H), 2.30-2.05 (m, 4H), 2.01 (s, 6H), 1.29 (s, 3H), 1.09 (s, 3H). LCMS (Method F): 473.5 [M−H]⁻. Example 115: ¹H NMR (400 MHz; DMSO-d₆) δ: 7.94 (s, 1H), 7.42-7.34 (m, 1H), 7.22-7.10 (m, 4H), 6.91 (d, 1H), 6.67 (d, 1H), 6.37 (dd, 1H), 4.58 (dd, 1H), 3.29-3.23 (m, 1H), 3.15 (dd, 1H), 2.80-2.72 (m, 4H), 2.68-2.60 (m, 1H), 2.30-2.05 (m, 4H), 2.01 (s, 6H), 1.29 (s, 3H), 1.08 (s, 3H). LCMS (Method F): 473.4 [M−H]⁻.

### Example 116: N-((1S)-1-(5-((1,1-Dimethyl-2,3-dihydro-1H-inden-2-yl)amino)pyridin-2-yl)-2,2,2-trifluoroethyl)-N-methyl-2-oxopiperidine-4-carboxamide—Isomer 1

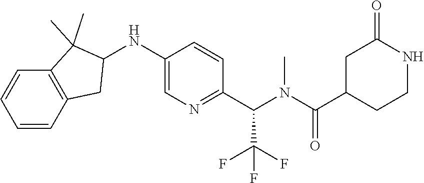 [SMILES: CN(C(=O)C1CCNC(=O)C1)[C@@H](c1ccc(NC2Cc3ccccc3C2(C)C)cn1)C(F)(F)F]

### Example 117: N-((1S)-1-(5-((1,1-Dimethyl-2,3-dihydro-1H-inden-2-yl)amino)pyridin-2-yl)-2,2,2-trifluoroethyl)-N-methyl-2-oxopiperidine-4-carboxamide—Isomer 2

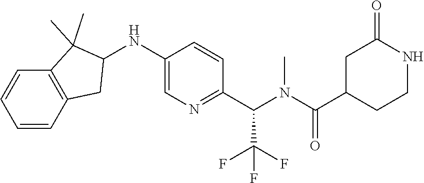 [SMILES: CN(C(=O)C1CCNC(=O)C1)[C@@H](c1ccc(NC2Cc3ccccc3C2(C)C)cn1)C(F)(F)F]

### Example 118: N-((1S)-1-(5-((1,1-Dimethyl-2,3-dihydro-1H-inden-2-yl)amino)pyridin-2-yl)-2,2,2-trifluoroethyl)-N-methyl-2-oxopiperidine-4-carboxamide—Isomer 3

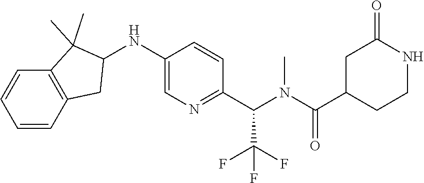 [SMILES: CN(C(=O)C1CCNC(=O)C1)[C@@H](c1ccc(NC2Cc3ccccc3C2(C)C)cn1)C(F)(F)F]

### Example 119: N-((1S)-1-(5-((1,1-Dimethyl-2,3-dihydro-1H-inden-2-yl)amino)pyridin-2-yl)-2,2,2-trifluoroethyl)-N-methyl-2-oxopiperidine-4-carboxamide—Isomer 4

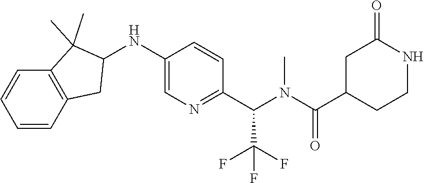 [SMILES: CN(C(=O)C1CCNC(=O)C1)[C@@H](c1ccc(NC2Cc3ccccc3C2(C)C)cn1)C(F)(F)F]

The title compounds were prepared in an analogous manner to Intermediate 3 from Intermediate 61 (0.50 g, 1.43 mmol), pyridine (2.5 mL), T3P® (50% EtOAc, 2.70 mL, 4.29 mmol) and 2-oxopiperidine-4-carboxylic acid (0.41 g, 2.86 mmol, CAS 24537-50-6) at RT for 16 h. Purified by preparative HPLC (XBridge C18, 19×250 mm×5 μm, flow rate: 19 mL/min, 10 mM aqueous NH₄HCO₃ with MeCN 10% to 60% over 3 min, held at 60% for 7 min, ramped to 100% over 0.1 min, held for 1.9 min). Chiral purification was carried out by preparative SFC (LUX-i-Amylose-3, 30×250 mm×5 μm, temp 30° C., Back Pressure 100 bar, flow rate: 100 g/min, 60% CO₂ with 40% (5 mM NH₄HCO₃ in MeOH) modifier) to provide Example 116 (41 mg) as Peak 1, Example 117 (20 mg) as Peak 2, Example 118 (47 mg) as Peak 3 and Example 119 (26 mg) as Peak 4. Example 116: ¹H NMR (400 MHz; DMSO-d₆) δ: 8.17-8.14 (m, 1H), 7.52-7.48 (m, 1H), 7.23-7.06 (m, 6H), 6.37 (dd, 1H), 6.21 (d, 1H), 4.04 (dd, 1H), 3.29-3.10 (m, 4H), 3.00 (s, 3H), 2.79-2.71 (m, 1H), 2.31-2.19 (m, 2H), 1.85-1.75 (m, 1H), 1.70-1.60 (m, 1H), 1.32 (s, 3H), 1.10 (s, 3H). LCMS (Method C): 475.5 [M+H]⁺. Example 117: ¹H NMR (400 MHz; DMSO-d₆) δ: 8.17-8.14 (m, 1H), 7.52-7.48 (m, 1H), 7.23-7.06 (m, 6H), 6.37 (dd, 1H), 6.20 (d, 1H), 4.04 (dd, 1H), 3.29-3.10 (m, 4H), 3.00 (s, 3H), 2.78-2.71 (m, 1H), 2.31-2.19 (m, 2H), 1.85-1.75 (m, 1H), 1.70-1.60 (m, 1H), 1.32 (s, 3H), 1.10 (s, 3H). LCMS (Method C): 475.5 [M+H]⁺. Example 118: ¹H NMR (400 MHz; DMSO-d₆) δ: 8.17-8.14 (m, 1H), 7.52-7.48 (m, 1H), 7.23-7.06 (m, 6H), 6.39 (dd, 1H), 6.22 (d, 1H), 4.04 (dd, 1H), 3.29-3.10 (m, 4H), 3.01 (s, 3H), 2.78-2.71 (m, 1H), 2.35-2.15 (m, 2H), 1.94-1.85 (m, 1H), 1.73-1.64 (m, 1H), 1.32 (s, 3H), 1.10 (s, 3H). LCMS (Method C): 475.5 [M+H]⁺. Example 119: ¹H NMR (400 MHz; DMSO-d₆) δ: 8.17-8.14 (m, 1H), 7.52-7.48 (m, 1H), 7.23-7.09 (m, 6H), 6.39 (dd, 1H), 6.22 (d, 1H), 4.04 (dd, 1H), 3.29-3.10 (m, 4H), 3.01 (s, 3H), 2.78-2.71 (m, 1H), 2.35-2.15 (m, 2H), 1.94-1.85 (m, 1H), 1.72-1.63 (m, 1H), 1.32 (s, 3H), 1.10 (s, 3H). LCMS (Method C): 475.5 [M+H]⁺.

### Example 120: N-((1S)-1-(6-((1,1-Dimethyl-2,3-dihydro-1H-inden-2-yl)amino)pyridin-3-yl)-2,2,2-trifluoroethyl)-N-methylquinuclidine-3-carboxamide—Isomer 1

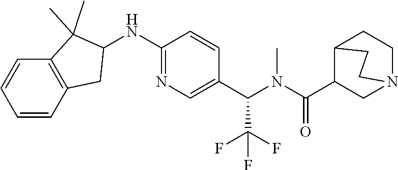 [SMILES: CN(C(=O)C1CN2CCC1CC2)[C@@H](c1ccc(NC2Cc3ccccc3C2(C)C)nc1)C(F)(F)F]

### Example 121: N-((1S)-1-(6-((1,1-Dimethyl-2,3-dihydro-1H-inden-2-yl)amino)pyridin-3-yl)-2,2,2-trifluoroethyl)-N-methylquinuclidine-3-carboxamide—Isomer 2

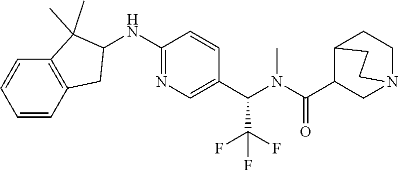 [SMILES: CN(C(=O)C1CN2CCC1CC2)[C@@H](c1ccc(NC2Cc3ccccc3C2(C)C)nc1)C(F)(F)F]

### Example 122: N-((1S)-1-(6-((1,1-Dimethyl-2,3-dihydro-1H-inden-2-yl)amino)pyridin-3-yl)-2,2,2-trifluoroethyl)-N-methylquinuclidine-3-carboxamide—Isomer 3

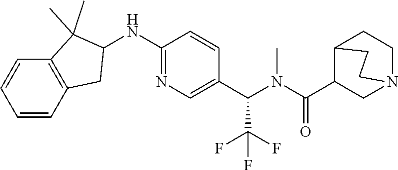 [SMILES: CN(C(=O)C1CN2CCC1CC2)[C@@H](c1ccc(NC2Cc3ccccc3C2(C)C)nc1)C(F)(F)F]

### Example 123: N-((1S)-1-(6-((1,1-Dimethyl-2,3-dihydro-1H-inden-2-yl)amino)pyridin-3-yl)-2,2,2-trifluoroethyl)-N-methylquinuclidine-3-carboxamide—Isomer 4

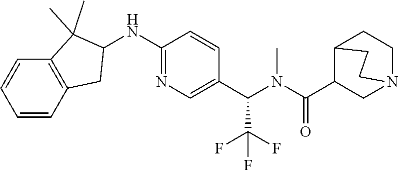 [SMILES: CN(C(=O)C1CN2CCC1CC2)[C@@H](c1ccc(NC2Cc3ccccc3C2(C)C)nc1)C(F)(F)F]

The title compounds were prepared in an analogous manner to Intermediate 3 from Intermediate 64 (0.35 g, 0.90 mmol), pyridine (3.5 mL), T3P® (50% EtOAc, 3.0 mL, 4.50 mmol) and quinuclidine-3-carboxylic acid (0.17 g, 1.09 mmol, CAS 75208-40-1) at RT for 16 h. Purified by preparative HPLC (XBridge C18, 19×250 mm×5 μm, flow rate: 18 mL/min, 10 mM aqueous NH₄HCO₃ with MeCN 35% to 70% over 8 min, held at 70% for 2 min, ramped to 100% over 0.1 min, held for 1.9 min). Chiral purification was carried out by preparative SFC (Chiralpak®-IG, 30×250 mm×5 μm, temp 30° C., Back Pressure 100 bar, flow rate: 100 g/min, 50% CO₂ with 50% (10 mM NH₄HCO₃ in MeOH/MeCN 1:1) modifier) then (Chiralpak®-IJ, 30×250 mm×5 μm, temp 30° C., Back Pressure 100 bar, flow rate: 100 g/min, 70% CO₂ with 30% (10 mM NH₄HCO₃ in MeOH/MeCN 1:1) modifier) to provide Example 120 (41 mg) as Peak 1, Example 121 (20 mg) as Peak 2, Example 122 (47 mg) as Peak 3 and Example 123 (26 mg) as Peak 4. Example 120: ¹H NMR (400 MHz; DMSO-d₆) δ: 7.93-7.91 (m, 1H), 7.33 (dd, 1H), 7.22-7.11 (m, 4H), 6.89 (d, 1H), 6.67 (d, 1H), 6.43 (dd, 1H), 4.57 (dd, 1H), 3.18 (dd, 1H), 3.10-3.03 (m, 1H), 2.95-2.88 (m, 1H), 2.87 (s, 3H), 2.79-2.58 (m, 5H), 1.84-1.80 (m, 1H), 1.65-1.45 (m, 3H), 1.70-1.60 (m, 1H), 1.28 (s, 3H), 1.27-1.22 (m, 1H), 1.08 (s, 3H). LCMS (Method A): 487.5 [M+H]⁺. Example 121: ¹H NMR (400 MHz; DMSO-d₆) δ: 7.90 (br s, 1H), 7.34 (dd, 1H), 7.22-7.11 (m, 4H), 6.91 (d, 1H), 6.67 (d, 1H), 6.43 (dd, 1H), 4.57 (dd, 1H), 3.15 (dd, 1H), 3.10-3.04 (m, 1H), 2.95-2.88 (m, 1H), 2.87 (s, 3H), 2.79-2.59 (m, 5H), 1.84-1.80 (m, 1H), 1.65-1.45 (m, 3H), 1.70-1.60 (m, 1H), 1.28 (s, 3H), 1.27-1.22 (m, 1H), 1.08 (s, 3H). LCMS (Method A): 487.5 [M+H]⁺. Example 122: ¹H NMR (400 MHz; DMSO-d₆) δ: 7.95 (br s, 1H), 7.38 (dd, 1H), 7.22-7.11 (m, 4H), 6.91 (d, 1H), 6.68 (d, 1H), 6.46 (dd, 1H), 4.59 (dd, 1H), 3.23-3.12 (m, 2H), 2.95-2.88 (m, 1H), 2.88 (s, 3H), 2.79-2.59 (m, 5H), 1.91-1.88 (m, 1H), 1.63-1.47 (m, 3H), 1.70-1.60 (m, 1H), 1.28 (s, 3H), 1.27-1.22 (m, 1H), 1.08 (s, 3H). LCMS (Method A): 487.5 [M+H]⁺. Example 123: ¹H NMR (400 MHz; DMSO-d₆) δ: 7.95 (br s, 1H), 7.38 (dd, 1H), 7.22-7.11 (m, 4H), 6.91 (d, 1H), 6.67 (d, 1H), 6.47 (dd, 1H), 4.58 (dd, 1H), 3.23-3.12 (m, 2H), 2.95-2.88 (m, 1H), 2.88 (s, 3H), 2.79-2.60 (m, 5H), 1.92-1.89 (m, 1H), 1.63-1.48 (m, 3H), 1.70-1.60 (m, 1H), 1.28 (s, 3H), 1.27-1.22 (m, 1H), 1.08 (s, 3H). LCMS (Method A): 487.5 [M+H]⁺.

### Example 124: N-((1S)-1-(5-((1,1-Dimethyl-2,3-dihydro-1H-inden-2-yl)amino)pyridin-2-yl)-2,2,2-trifluoroethyl)-N-methylpiperidine-3-carboxamide—Isomer 1

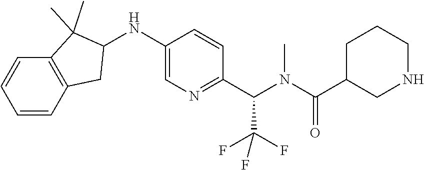 [SMILES: CN(C(=O)C1CCCNC1)[C@@H](c1ccc(NC2Cc3ccccc3C2(C)C)cn1)C(F)(F)F]

### Example 125: N-((1S)-1-(5-((1,1-Dimethyl-2,3-dihydro-1H-inden-2-yl)amino)pyridin-2-yl)-2,2,2-trifluoroethyl)-N-methylpiperidine-3-carboxamide—Isomer 2

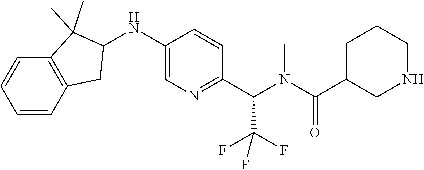 [SMILES: CN(C(=O)C1CCCNC1)[C@@H](c1ccc(NC2Cc3ccccc3C2(C)C)cn1)C(F)(F)F]

### Example 126: N-((1S)-1-(5-((1,1-Dimethyl-2,3-dihydro-1H-inden-2-yl)amino)pyridin-2-yl)-2,2,2-trifluoroethyl)-N-methylpiperidine-3-carboxamide—Isomer 3

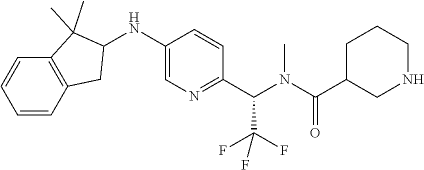 [SMILES: CN(C(=O)C1CCCNC1)[C@@H](c1ccc(NC2Cc3ccccc3C2(C)C)cn1)C(F)(F)F]

### Example 127: N-((1S)-1-(5-((1,1-Dimethyl-2,3-dihydro-1H-inden-2-yl)amino)pyridin-2-yl)-2,2,2-trifluoroethyl)-N-methylpiperidine-3-carboxamide—Isomer 4

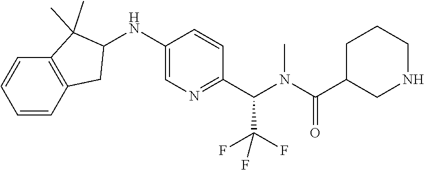 [SMILES: CN(C(=O)C1CCCNC1)[C@@H](c1ccc(NC2Cc3ccccc3C2(C)C)cn1)C(F)(F)F]

Intermediate 71 (0.13 g) was purified by chiral preparative SFC (Chiralpak®-IG, 30×250 mm×5 μm, temp 30° C., Back Pressure 100 bar, flow rate: 100 g/min, 65% CO₂ with 35% (5 mM NH₄HCO₃ in MeOH/MeCN 1:1) modifier) then (LUX Cellulose-02, 30×250 mm×5 μm, temp 30° C., Back Pressure 100 bar, flow rate: 100 g/min, 60% CO₂ with 40% 5 mM NH₄HCO₃ in MeOH modifier) to provide Example 124 (16 mg) as Peak 1, Example 125 (17 mg) as Peak 2, Example 126 (24 mg) as Peak 3 and Example 127 (16 mg) as Peak 4. Example 124: ¹H NMR (400 MHz; DMSO-d₆) δ: 8.17-8.12 (m, 1H), 7.22-7.12 (m, 5H), 7.06 (d, 1H), 6.37 (dd, 1H), 6.20 (d, 1H), 4.04 (dd, 1H), 3.20 (dd, 1H), 2.99 (s, 3H), 2.96-2.85 (m, 2H), 2.82-2.65 (m, 2H), 2.53-2.47 (m, 2H), 1.80-1.66 (m, 1H), 1.65-1.37 (m, 3H), 1.32 (s, 3H), 1.23 (br s, 1H), 1.09 (s, 3H). LCMS (Method A): 461.5 [M+H]⁺. Example 125: ¹H NMR (400 MHz; DMSO-d₆) δ: 8.17-8.12 (m, 1H), 7.22-7.12 (m, 5H), 7.06 (d, 1H), 6.37 (dd, 1H), 6.20 (d, 1H), 4.04 (dd, 1H), 3.20 (dd, 1H), 3.06-2.90 (m, 5H), 2.88-2.80 (m, 1H), 2.74 (dd, 1H), 2.70-2.55 (m, 2H), 1.81-1.44 (m, 4H), 1.32 (s, 3H), 1.23 (br s, 1H), 1.10 (s, 3H). LCMS (Method A): 461.5 [M+H]⁺. Example 126: ¹H NMR (400 MHz; DMSO-d₆) δ: 8.17-8.12 (m, 1H), 7.22-7.12 (m, 5H), 7.06 (d, 1H), 6.37 (dd, 1H), 6.20 (d, 1H), 4.04 (dd, 1H), 3.20 (dd, 1H), 2.99 (s, 3H), 2.93-2.83 (m, 2H), 2.82-2.65 (m, 2H), 2.53-2.47 (m, 2H), 1.88-1.80 (m, 1H), 1.62-1.37 (m, 3H), 1.32 (s, 3H), 1.24 (br s, 1H), 1.10 (s, 3H). LCMS (Method H): 461.5 [M+H]⁺. Example 127: ¹H NMR (400 MHz; DMSO-d₆) δ: 8.17-8.12 (m, 1H), 7.22-7.11 (m, 5H), 7.06 (d, 1H), 6.37 (dd, 1H), 6.20 (d, 1H), 4.04 (dd, 1H), 3.20 (dd, 1H), 2.99 (s, 3H), 2.96-2.85 (m, 2H), 2.80-2.65 (m, 2H), 2.53-2.47 (m, 2H), 1.88-1.79 (m, 1H), 1.60-1.37 (m, 3H), 1.32 (s, 3H), 1.24 (br s, 1H), 1.10 (s, 3H). LCMS (Method H): 461.5 [M+H]⁺.

### Example 128: N-((1S)-1-(5-((1,1-Dimethyl-2,3-dihydro-1H-inden-2-yl)amino)pyridin-2-yl)-2,2,2-trifluoroethyl)-N,1-dimethylpiperidine-3-carboxamide—Isomer 1

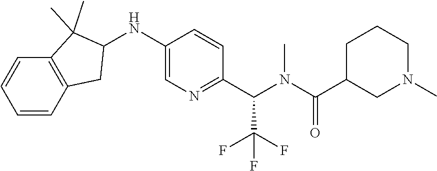 [SMILES: CN1CCCC(C(=O)N(C)[C@@H](c2ccc(NC3Cc4ccccc4C3(C)C)cn2)C(F)(F)F)C1]

### Example 129: N-((1S)-1-(5-((1,1-Dimethyl-2,3-dihydro-1H-inden-2-yl)amino)pyridin-2-yl)-2,2,2-trifluoroethyl)-N,1-dimethylpiperidine-3-carboxamide—Isomer 2

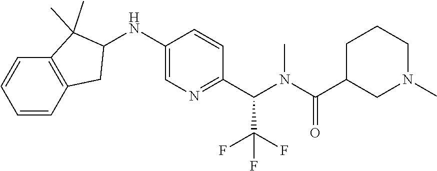 [SMILES: CN1CCCC(C(=O)N(C)[C@@H](c2ccc(NC3Cc4ccccc4C3(C)C)cn2)C(F)(F)F)C1]

### Example 130: N-((1S)-1-(5-((1,1-Dimethyl-2,3-dihydro-1H-inden-2-yl)amino)pyridin-2-yl)-2,2,2-trifluoroethyl)-N,1-dimethylpiperidine-3-carboxamide—Isomer 3

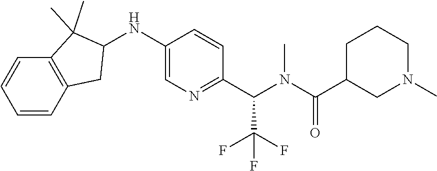 [SMILES: CN1CCCC(C(=O)N(C)[C@@H](c2ccc(NC3Cc4ccccc4C3(C)C)cn2)C(F)(F)F)C1]

### Example 131: N-((1S)-1-(5-((1,1-Dimethyl-2,3-dihydro-1H-inden-2-yl)amino)pyridin-2-yl)-2,2,2-trifluoroethyl)-N,1-dimethylpiperidine-3-carboxamide—Isomer 4

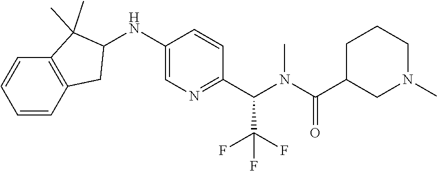 [SMILES: CN1CCCC(C(=O)N(C)[C@@H](c2ccc(NC3Cc4ccccc4C3(C)C)cn2)C(F)(F)F)C1]

The title compounds were prepared in an analogous manner to Intermediate 3 from Intermediate 61 (0.25 g, 0.72 mmol), pyridine (0.3 mL), T3P® (50% EtOAc, 3.2 mL, 5.01 mmol) and 1-methylpiperidine-3-carboxylic acid (0.15 g, 1.07 mmol, CAS 5657-70-5) at RT for 16 h. Purified by achiral SFC (YMCPAK DIOL, 30×250 mm×5 μm, temp 30° C., Back Pressure 100 bar, flow rate: 100 g/min, 75% CO₂ with 25% 5 mM NH₄HCO₃ in MeOH modifier). Chiral purification was carried out by preparative SFC (LUX Cellulose-2, 30×250 mm×5 μm, temp 30° C., Back Pressure 100 bar, flow rate: 100 g/min, 75% CO₂ with 25% 10 mM NH₄HCO₃ in MeOH modifier) to provide Example 128 (22 mg) as Peak 1, Example 129 (29 mg) as Peak 2, Example 130 (31 mg) as Peak 3 and Example 131 (26 mg) as Peak 4. Example 128: ¹H NMR (400 MHz; DMSO-d₆) δ: 8.14 (d, 1H), 7.22-7.12 (m, 5H), 7.06 (d, 1H), 6.37 (dd, 1H), 6.20 (d, 1H), 4.04 (dd, 1H), 3.19 (dd, 1H), 3.00 (s, 3H), 2.90-2.70 (m, 4H), 2.16 (s, 3H), 1.88 (t, 1H), 1.82-1.74 (m, 1H), 1.70-1.59 (m, 2H), 1.56-1.45 (m, 1H), 1.37-1.23 (m, 4H), 1.10 (s, 3H). LCMS (Method A): 475.4 [M+H]⁺. Example 129: ¹H NMR (400 MHz; DMSO-d₆) δ: 8.14 (d, 1H), 7.22-7.12 (m, 5H), 7.07 (d, 1H), 6.37 (dd, 1H), 6.20 (d, 1H), 4.04 (dd, 1H), 3.19 (dd, 1H), 3.00 (s, 3H), 2.92-2.82 (m, 1H), 2.78-2.70 (m, 3H), 2.15 (s, 3H), 1.96-1.88 (m, 1H), 1.82-1.74 (m, 2H), 1.68-1.49 (m, 2H), 1.32 (s, 3H), 1.28-1.22 (m, 1H), 1.10 (s, 3H). LCMS (Method A): 475.4 [M+H]⁺. Example 130: ¹H NMR (400 MHz; DMSO-d₆) δ: 8.14 (d, 1H), 7.22-7.12 (m, 5H), 7.06 (d, 1H), 6.37 (dd, 1H), 6.20 (d, 1H), 4.04 (dd, 1H), 3.19 (dd, 1H), 3.00 (s, 3H), 2.90-2.70 (m, 4H), 2.16 (s, 3H), 1.92-1.75 (m, 2H), 1.72-1.59 (m, 2H), 1.56-1.45 (m, 1H), 1.37-1.23 (m, 4H), 1.10 (s, 3H). LCMS (Method A): 475.5 [M+H]⁺. Example 131: ¹H NMR (400 MHz; DMSO-d₆) δ: 8.14 (d, 1H), 7.22-7.12 (m, 5H), 7.07 (d, 1H), 6.37 (dd, 1H), 6.20 (d, 1H), 4.04 (dd, 1H), 3.19 (dd, 1H), 3.00 (s, 3H), 2.90-2.82 (m, 1H), 2.78-2.68 (m, 3H), 2.14 (s, 3H), 1.96-1.88 (m, 1H), 1.82-1.74 (m, 2H), 1.68-1.49 (m, 2H), 1.32 (s, 3H), 1.28-1.22 (m, 1H), 1.10 (s, 3H). LCMS (Method A): 475.5 [M+H]⁺.

### Example 132: N-((1S)-1-(5-((1,1-Dimethyl-2,3-dihydro-1H-inden-2-yl)amino)pyridin-2-yl)-2,2,2-trifluoroethyl)-N,1-dimethylpiperidine-4-carboxamide—Isomer 1

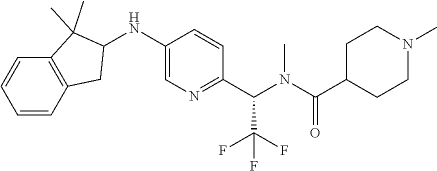 [SMILES: CN1CCC(C(=O)N(C)[C@@H](c2ccc(NC3Cc4ccccc4C3(C)C)cn2)C(F)(F)F)CC1]

### Example 133: N-((1S)-1-(5-((1,1-Dimethyl-2,3-dihydro-1H-inden-2-yl)amino)pyridin-2-yl)-2,2,2-trifluoroethyl)-N,1-dimethylpiperidine-4-carboxamide—Isomer 2

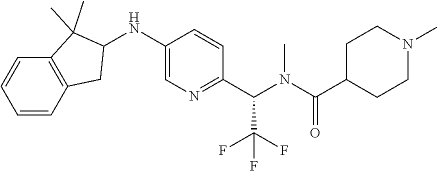 [SMILES: CN1CCC(C(=O)N(C)[C@@H](c2ccc(NC3Cc4ccccc4C3(C)C)cn2)C(F)(F)F)CC1]

The title compounds were prepared in an analogous manner to Intermediate 3 from Intermediate 61 (0.20 g, 0.57 mmol), pyridine (1.0 mL), T3P® (50% EtOAc, 1.27 g, 4.00 mmol) and 1-methylpiperidine-4-carboxylic acid (0.24 g, 1.71 mmol, CAS 68947-43-3) at RT for 16 h. Purified by preparative HPLC (XBridge C18, 19×250 mm×5 μm, flow rate: 19 mL/min, 10 mM aqueous NH₄HCO₃ with MeCN 45% to 70% over 8 min, held at 70% for 2 min, ramped to 100% over 0.1 min, held for 1.9 min). Chiral purification was carried out by preparative SFC (LUX Cellulose-2, 30×250 mm×5 μm, temp 30° C., Back Pressure 100 bar, flow rate: 100 g/min, 60% CO₂ with 40% 5 mM NH₄HCO₃ in MeOH modifier) to provide Example 132 (51 mg) as Peak 1 and Example 133 (68 mg) as Peak 2. Example 132: ¹H NMR (400 MHz; DMSO-d₆) δ: 8.14 (d, 1H), 7.22-7.12 (m, 5H), 7.06 (d, 1H), 6.38 (dd, 1H), 6.19 (d, 1H), 4.04 (dd, 1H), 3.21 (dd, 1H), 2.99 (s, 3H), 2.81-2.55 (m, 4H), 2.14 (s, 3H), 1.94-1.83 (m, 2H), 1.72-1.52 (m, 4H), 1.32 (s, 3H), 1.10 (s, 3H). LCMS (Method A): 475.5 [M+H]⁺. Example 133: ¹H NMR (400 MHz; DMSO-d₆) δ: 8.14 (d, 1H), 7.22-7.12 (m, 5H), 7.06 (d, 1H), 6.38 (dd, 1H), 6.20 (d, 1H), 4.04 (dd, 1H), 3.19 (dd, 1H), 2.98 (s, 3H), 2.81-2.55 (m, 4H), 2.14 (s, 3H), 1.94-1.83 (m, 2H), 1.72-1.52 (m, 4H), 1.32 (s, 3H), 1.10 (s, 3H). LCMS (Method A): 475.4 [M+H]⁺.

### Example 134: N-((1S)-1-(5-((1,1-Dimethyl-2,3-dihydro-1H-inden-2-yl)amino)pyridin-2-yl)-2,2,2-trifluoroethyl)-N-methyl-5-oxomorpholine-2-carboxamide—Isomer 1

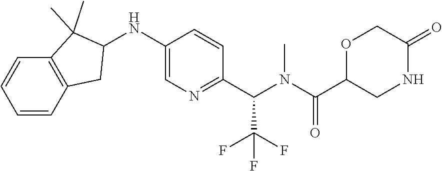 [SMILES: CN(C(=O)C1CNC(=O)CO1)[C@@H](c1ccc(NC2Cc3ccccc3C2(C)C)cn1)C(F)(F)F]

### Example 135: N-((1S)-1-(5-((1,1-Dimethyl-2,3-dihydro-1H-inden-2-yl)amino)pyridin-2-yl)-2,2,2-trifluoroethyl)-N-methyl-5-oxomorpholine-2-carboxamide—Isomer 2

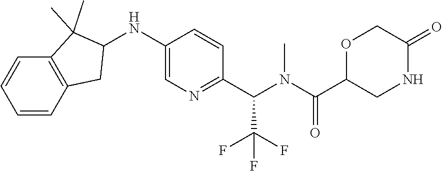 [SMILES: CN(C(=O)C1CNC(=O)CO1)[C@@H](c1ccc(NC2Cc3ccccc3C2(C)C)cn1)C(F)(F)F]

### Example 136: N-((1S)-1-(5-((1,1-Dimethyl-2,3-dihydro-1H-inden-2-yl)amino)pyridin-2-yl)-2,2,2-trifluoroethyl)-N-methyl-5-oxomorpholine-2-carboxamide—Isomer 3

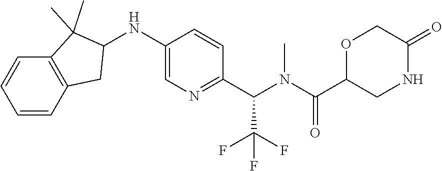 [SMILES: CN(C(=O)C1CNC(=O)CO1)[C@@H](c1ccc(NC2Cc3ccccc3C2(C)C)cn1)C(F)(F)F]

### Example 137: N-((1S)-1-(5-((1,1-Dimethyl-2,3-dihydro-1H-inden-2-yl)amino)pyridin-2-yl)-2,2,2-trifluoroethyl)-N-methyl-5-oxomorpholine-2-carboxamide—Isomer 4

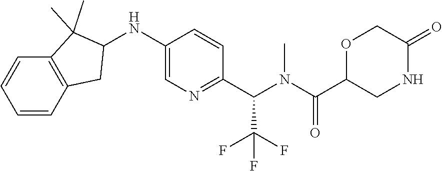 [SMILES: CN(C(=O)C1CNC(=O)CO1)[C@@H](c1ccc(NC2Cc3ccccc3C2(C)C)cn1)C(F)(F)F]

The title compounds were prepared in an analogous manner to Intermediate 3 from Intermediate 61 (0.11 g, 0.32 mmol), pyridine (0.55 mL), T3P® (50% EtOAc, 0.6 mL, 0.94 mmol) and Intermediate 68 (90 mg, 0.63 mmol) at RT for 16 h. Purified by column chromatography (silica gel, eluting 100% EtOAc) followed by preparative HPLC (XBridge C18, 19×150 mm×5 μm, flow rate: 18 mL/min, 10 mM aqueous NH₄HCO₃ with MeCN 45% to 60% over 13 min, ramped to 98% over 0.1 min, held for 1.9 min). Chiral purification was carried out by preparative SFC (Chiralpak®-AD-H, 30×250 mm×5 μm, temp 30° C., Back Pressure 100 bar, flow rate: 100 g/min, 65% CO₂ with 35% 5 mM NH₄HCO₃ in MeOH modifier) to provide Example 134 (16 mg) as Peak 1, Example 135 (13 mg) as Peak 2, Example 136 (21 mg) as Peak 3 and Example 137 (24 mg) as Peak 4. Example 134: ¹H NMR (400 MHz; DMSO-d₆) δ: 8.15 (s, 1H), 8.12-8.06 (m, 1H), 7.22-7.12 (m, 6H), 6.31 (dd, 1H), 6.24 (d, 1H), 4.77-4.71 (m, 1H), 4.21-4.02 (m, 3H), 3.56-3.48 (m, 1H), 3.30-3.25 (m, 1H), 3.26 (dd, 1H), 3.04 (s, 3H), 2.80-2.70 (m, 1H), 1.32 (s, 3H), 1.10 (s, 3H). LCMS (Method C): 477.4 [M+H]⁺. Example 135: ¹H NMR (400 MHz; DMSO-d₆) δ: 8.15 (s, 1H), 8.12-8.06 (m, 1H), 7.22-7.12 (m, 6H), 6.31 (dd, 1H), 6.24 (d, 1H), 4.77-4.71 (m, 1H), 4.21-4.02 (m, 3H), 3.56-3.48 (m, 1H), 3.30-3.25 (m, 1H), 3.26 (dd, 1H), 3.04 (s, 3H), 2.80-2.70 (m, 1H), 1.32 (s, 3H), 1.10 (s, 3H). LCMS (Method C): 477.4 [M+H]⁺. Example 136: ¹H NMR (400 MHz; DMSO-d₆) δ: 8.17-8.14 (m, 1H), 8.11-8.07 (m, 1H), 7.22-7.08 (m, 6H), 6.32-6.22 (m, 2H), 4.82-4.75 (m, 1H), 4.18-4.02 (m, 3H), 3.53-3.48 (m, 1H), 3.30-3.25 (m, 1H), 3.19 (dd, 1H), 3.04 (s, 3H), 2.80-2.70 (m, 1H), 1.32 (s, 3H), 1.10 (s, 3H). LCMS (Method C): 477.5 [M+H]⁺. Example 137: ¹H NMR (400 MHz; DMSO-d₆) δ: 8.16 (br s, 1H), 8.08 (br s, 1H), 7.22-7.08 (m, 6H), 6.32-6.22 (m, 2H), 4.81-4.75 (m, 1H), 4.18-4.02 (m, 3H), 3.53-3.48 (m, 1H), 3.30-3.18 (m, 2H), 3.04 (s, 3H), 2.80-2.70 (m, 1H), 1.32 (s, 3H), 1.10 (s, 3H). LCMS (Method C): 477.4 [M+H]⁺.

### Example 138: N-((1S)-1-(5-((1,1-Dimethyl-2,3-dihydro-1H-inden-2-yl)amino)pyridin-2-yl)-2,2,2-trifluoroethyl)-N-methylquinuclidine-3-carboxamide—Isomer 1

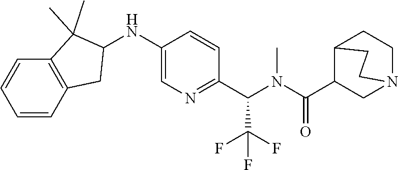 [SMILES: CN(C(=O)C1CN2CCC1CC2)[C@@H](c1ccc(NC2Cc3ccccc3C2(C)C)cn1)C(F)(F)F]

### Example 139: N-((1S)-1-(5-((1,1-Dimethyl-2,3-dihydro-1H-inden-2-yl)amino)pyridin-2-yl)-2,2,2-trifluoroethyl)-N-methylquinuclidine-3-carboxamide—Isomer 2

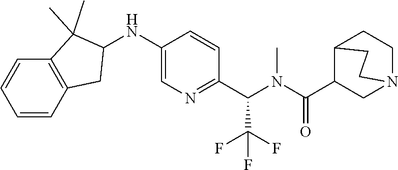 [SMILES: CN(C(=O)C1CN2CCC1CC2)[C@@H](c1ccc(NC2Cc3ccccc3C2(C)C)cn1)C(F)(F)F]

### Example 140: N-((1S)-1-(5-((1,1-Dimethyl-2,3-dihydro-1H-inden-2-yl)amino)pyridin-2-yl)-2,2,2-trifluoroethyl)-N-methylquinuclidine-3-carboxamide—Isomer 3

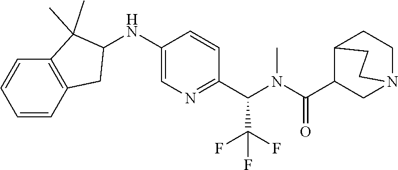 [SMILES: CN(C(=O)C1CN2CCC1CC2)[C@@H](c1ccc(NC2Cc3ccccc3C2(C)C)cn1)C(F)(F)F]

### Example 141: N-((1S)-1-(5-((1,1-Dimethyl-2,3-dihydro-1H-inden-2-yl)amino)pyridin-2-yl)-2,2,2-trifluoroethyl)-N-methylquinuclidine-3-carboxamide—Isomer 4

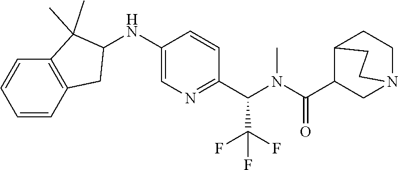 [SMILES: CN(C(=O)C1CN2CCC1CC2)[C@@H](c1ccc(NC2Cc3ccccc3C2(C)C)cn1)C(F)(F)F]

The title compounds were prepared in an analogous manner to Intermediate 3 from Intermediate 61 (0.10 g, 0.29 mmol), pyridine (0.5 mL), T3P® (50% EtOAc, 1.82 mL, 2.86 mmol) and quinuclidine-3-carboxylic acid (67 mg, 0.43 mmol, CAS 75208-40-1) at RT for 16 h. Purified by preparative HPLC (Sunfire C18, 19×150 mm×5 μm, flow rate: 18 mL/min, 0.1% formic acid in water with MeCN 5% to 30% over 6 min, held at 30% for 12 min, ramped to 100% over 0.1 min, held for 1.9 min). Chiral purification was carried out by preparative SFC (Chiralpak®-IG, 30×250 mm×5 μm, temp 30° C., Back Pressure 100 bar, flow rate: 100 g/min, 50% CO₂ with 50% 10 mM NH₄HCO₃ in MeOH modifier) to provide Example 138 (18 mg) as Peak 1, Example 139 (25 mg) as Peak 2, Example 140 (7.8 mg) as Peak 3 and Example 141 (8.8 mg) as Peak 4. Example 138: ¹H NMR (400 MHz; DMSO-d₆) δ: 8.15-8.12 (m, 1H), 7.22-7.11 (m, 5H), 7.03 (d, 1H), 6.43 (dd, 1H), 6.17 (d, 1H), 4.03 (dd, 1H), 3.20 (dd, 1H), 3.11-2.90 (m, 6H), 2.80-2.60 (m, 5H), 1.87 (s, 1H), 1.65-1.46 (m, 3H), 1.31 (s, 3H), 1.28-1.22 (m, 1H), 1.10 (s, 3H). LCMS (Method A): 487.4 [M+H]⁺. Example 139: ¹H NMR (400 MHz; DMSO-d₆) δ: 8.15-8.12 (m, 1H), 7.22-7.08 (m, 6H), 6.47 (dd, 1H), 6.20 (d, 1H), 4.03 (dd, 1H), 3.23-3.17 (m, 2H), 2.96-2.91 (m, 3H), 2.81-2.57 (m, 7H), 1.93-1.89 (m, 1H), 1.65-1.49 (m, 3H), 1.32 (s, 3H), 1.30-1.22 (m, 1H), 1.10 (s, 3H). LCMS (Method A): 487.5 [M+H]⁺. Example 140: ¹H NMR (400 MHz; DMSO-d₆) δ: 8.15-8.12 (m, 1H), 7.22-7.11 (m, 5H), 7.03 (d, 1H), 6.43 (dd, 1H), 6.17 (d, 1H), 4.03 (dd, 1H), 3.23-3.14 (m, 2H), 3.07-2.94 (m, 5H), 2.88-2.65 (m, 5H), 1.87 (s, 1H), 1.67-1.48 (m, 3H), 1.32 (s, 3H), 1.28-1.22 (m, 1H), 1.10 (s, 3H). LCMS (Method A): 487.4 [M+H]⁺. Example 141: ¹H NMR (400 MHz; DMSO-d₆) δ: 8.15-8.12 (m, 1H), 7.22-7.08 (m, 6H), 6.47 (dd, 1H), 6.21 (d, 1H), 4.04 (dd, 1H), 3.29-3.25 (m, 1H), 2.18 (dd, 1H), 3.05-2.98 (m, 1H), 2.96 (s, 3H), 2.90-2.65 (m, 6H), 1.93-1.89 (m, 1H), 1.65-1.49 (m, 3H), 1.41-1.33 (m, 1H), 1.32 (s, 3H), 1.10 (s, 3H). LCMS (Method A): 487.4 [M+H]⁺.

### Example 142: N-((1S)-1-(5-((1,1-Dimethyl-2,3-dihydro-1H-inden-2-yl)amino)pyridin-2-yl)-2,2,2-trifluoroethyl)-N-methylthiomorpholine-4-carboxamide 1,1-dioxide—Isomer 1

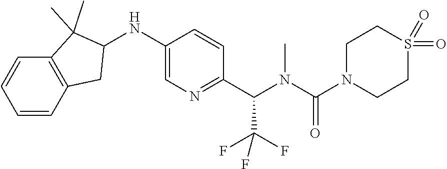 [SMILES: CN(C(=O)N1CCS(=O)(=O)CC1)[C@@H](c1ccc(NC2Cc3ccccc3C2(C)C)cn1)C(F)(F)F]

### Example 143: N-((1S)-1-(5-((1,1-Dimethyl-2,3-dihydro-1H-inden-2-yl)amino)pyridin-2-yl)-2,2,2-trifluoroethyl)-N-methylthiomorpholine-4-carboxamide 1,1-dioxide—Isomer 2

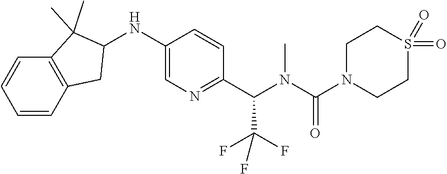 [SMILES: CN(C(=O)N1CCS(=O)(=O)CC1)[C@@H](c1ccc(NC2Cc3ccccc3C2(C)C)cn1)C(F)(F)F]

To a stirred mixture of Intermediate 61 (0.10 g, 0.29 mmol) and thiomorpholine 1,1-dioxide (39 mg, 0.29 mmol, CAS 39093-93-1) in DCM (5 mL) was added triethylamine (87 mg, 0.86 mmol) at 0° C. After 10 min, a solution of triphosgene (85 mg, 0.29 mmol) in DCM (0.2 mL) was added at 0° C. then stirred at RT for 1 h. The mixture was heated at 80° C. in a sealed tube then allowed to cool. The mixture was poured into water and extracted with DCM. The organic layer was dried over Na₂SO₄, filtered and concentrated under reduced pressure. Purified by preparative HPLC (X-Select C18, 19×150 mm×5 μm, flow rate: 16 mL/min, 10 mM aqueous NH₄HCO₃ containing 0.1% aqueous ammonia with MeCN 70% 10 min, ramped to 100% over 0.1 min and held for 10 min). Chiral purification was carried out by preparative SFC (Chiralpak®-IG, 30×250 mm×5 μm, temp 30° C., Back Pressure 100 bar, flow rate: 100 g/min, 55% CO₂ with 45% 5 mM NH₄HCO₃ in MeOH modifier) to provide Example 142 (16 mg) as Peak 1 and Example 143 (24 mg) as Peak 2. Example 142: ¹H NMR (400 MHz; DMSO-d₆) δ: 8.14 (d, 1H), 7.23-7.12 (m, 6H), 6.20 (d, 1H), 5.78 (dd, 1H), 4.04 (dd, 1H), 3.62-3.57 (m, 4H), 3.29-3.10 (m, 5H), 2.87 (s, 3H), 2.74 (dd, 1H), 1.32 (s, 3H), 1.09 (s, 3H). LCMS (Method A): 511.4 [M+H]⁺. Example 143: ¹H NMR (400 MHz; DMSO-d₆) δ: 8.14 (d, 1H), 7.23-7.12 (m, 6H), 6.19 (d, 1H), 5.78 (dd, 1H), 4.04 (dd, 1H), 3.62-3.57 (m, 4H), 3.29-3.10 (m, 5H), 2.88 (s, 3H), 2.74 (dd, 1H), 1.32 (s, 3H), 1.09 (s, 3H). LCMS (Method A): 511.4 [M+H]⁺.

### Example 144: N-((1S)-1-(6-((1,1-Dimethyl-2,3-dihydro-1H-inden-2-yl)amino)pyridin-3-yl)-2,2,2-trifluoroethyl)-N,1-dimethylpiperidine-4-carboxamide—Isomer 1

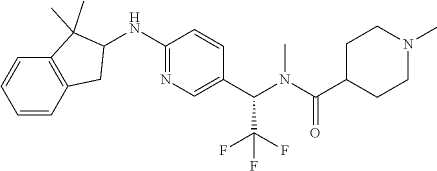 [SMILES: CN1CCC(C(=O)N(C)[C@@H](c2ccc(NC3Cc4ccccc4C3(C)C)nc2)C(F)(F)F)CC1]

### Example 145: N-((1S)-1-(6-((1,1-Dimethyl-2,3-dihydro-1H-inden-2-yl)amino)pyridin-3-yl)-2,2,2-trifluoroethyl)-N,1-dimethylpiperidine-4-carboxamide—Isomer 2

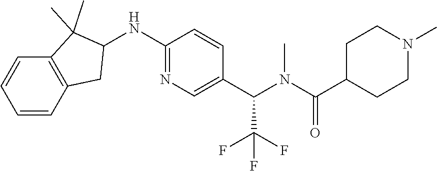 [SMILES: CN1CCC(C(=O)N(C)[C@@H](c2ccc(NC3Cc4ccccc4C3(C)C)nc2)C(F)(F)F)CC1]

The title compounds were prepared in an analogous manner to Intermediate 3 from Intermediate 64 (0.37 g, 0.96 mmol), pyridine (1.85 mL), T3P® (50% EtOAc, 4.3 mL, 6.70 mmol) and 1-methylpiperidine-4-carboxylic acid (0.41 g, 2.88 mmol, CAS 68947-43-3) at RT for 16 h. Purified by preparative HPLC (XBridge C18, 19×250 mm×5 μm, flow rate: 18 mL/min, 10 mM aqueous NH₄HCO₃ with MeCN 20% to 66% over 7 min, held at 66% for 5.7 min, ramped to 98% over 0.1 min, held for 11.2 min). Chiral purification was carried out by preparative SFC (Chiralpak®-IG, 30×250 mm×5 μm, temp 30° C., Back Pressure 100 bar, flow rate: 100 g/min, 60% CO₂ with 40% 10 mM NH₄HCO₃ in MeOH modifier) to provide Example 144 (30 mg) as Peak 1 and Example 145 (34 mg) as Peak 2. Example 144: ¹H NMR (400 MHz; DMSO-d₆) δ: 7.93 (s, 1H), 7.42-7.32 (m, 1H), 7.22-7.10 (m, 4H), 6.91 (d, 1H), 6.67 (d, 1H), 6.37 (dd, 1H), 4.58 (dd, 1H), 3.15 (dd, 1H), 2.91 (s, 3H), 2.84-2.57 (m, 4H), 2.19 (s, 3H), 1.98 (br s, 2H), 1.73-1.55 (m, 4H), 1.28 (s, 3H), 1.08 (s, 3H). LCMS (Method H): 475.5 [M+H]⁺. Example 145: ¹H NMR (400 MHz; DMSO-d₆) δ: 7.93 (s, 1H), 7.42-7.32 (m, 1H), 7.22-7.10 (m, 4H), 6.91 (d, 1H), 6.67 (d, 1H), 6.37 (dd, 1H), 4.58 (dd, 1H), 3.15 (dd, 1H), 2.91 (s, 3H), 2.84-2.57 (m, 4H), 2.34-2.05 (m, 5H), 1.73-1.55 (m, 4H), 1.29 (s, 3H), 1.08 (s, 3H). LCMS (Method H): 475.5 [M+H]⁺.

### Example 146: N-((1S)-1-(5-((1,1-Dimethyl-2,3-dihydro-1H-inden-2-yl)amino)pyridin-2-yl)-2,2,2-trifluoroethyl)-N-methylpiperidine-4-carboxamide—Isomer 1

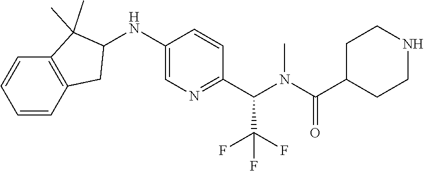 [SMILES: CN(C(=O)C1CCNCC1)[C@@H](c1ccc(NC2Cc3ccccc3C2(C)C)cn1)C(F)(F)F]

### Example 147: N-((1S)-1-(5-((1,1-Dimethyl-2,3-dihydro-1H-inden-2-yl)amino)pyridin-2-yl)-2,2,2-trifluoroethyl)-N-methylpiperidine-4-carboxamide—Isomer 2

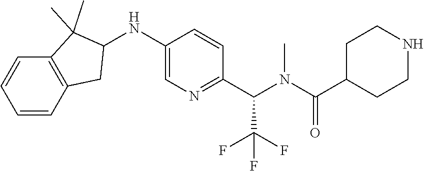 [SMILES: CN(C(=O)C1CCNCC1)[C@@H](c1ccc(NC2Cc3ccccc3C2(C)C)cn1)C(F)(F)F]

The title compounds were prepared from Intermediate 72 (0.15 g, 0.27 mmol) in 1,4-dioxane (1.5 mL) at 0° C. treated with 4M HCl in 1,4-dioxane (0.75 mL, 3.0 mmol) then stirred at RT for 1 h. The mixture was concentrated under reduced pressure then diluted with saturated aqueous NaHCO₃ and extracted with EtOAc. The organic layer was dried over Na₂SO₄, filtered and concentrated under reduced pressure. Purified by achiral preparative SFC (YMC Pack Diol, 30×250 mm×5 μm, temp 30° C., Back Pressure 100 bar, flow rate: 100 g/min, 75% CO₂ with 25% 10 mM NH₄HCO₃ in MeOH modifier). Chiral purification was carried out by preparative SFC (Chiralpak®-IG, 30×250 mm×5 μm, temp 30° C., Back Pressure 100 bar, flow rate: 100 g/min, 60% CO₂ with 40% 10 mM NH₄HCO₃ in MeOH modifier) to provide Example 146 (22 mg) as Peak 1 and Example 147 (22 mg) as Peak 2. Example 146: ¹H NMR (400 MHz; DMSO-d₆) δ: 8.14 (d, 1H), 7.22-7.12 (m, 5H), 7.06 (d, 1H), 6.39 (dd, 1H), 6.01 (d, 1H), 4.04 (dd, 1H), 3.20 (dd, 1H), 3.00-2.90 (m, 5H), 2.79-2.64 (m, 4H), 1.73-1.40 (m, 4H), 1.32 (s, 3H), 1.23 (br s, 1H), 1.10 (s, 3H). LCMS (Method A): 461.4 [M+H]⁺. Example 147: ¹H NMR (400 MHz; DMSO-d₆) δ: 8.14 (d, 1H), 7.22-7.12 (m, 5H), 7.06 (d, 1H), 6.39 (dd, 1H), 6.01 (d, 1H), 4.04 (dd, 1H), 3.20 (dd, 1H), 3.00-2.90 (m, 5H), 2.79-2.64 (m, 4H), 1.73-1.40 (m, 4H), 1.32 (s, 3H), 1.23 (br s, 1H), 1.10 (s, 3H). LCMS (Method A): 461.4 [M+H]⁺.

### Example 148: N-((1S)-1-(6-((1,1-Dimethyl-2,3-dihydro-1H-inden-2-yl)amino)pyridin-3-yl)-2,2,2-trifluoroethyl)-N-methylpiperidine-4-carboxamide—Isomer 1

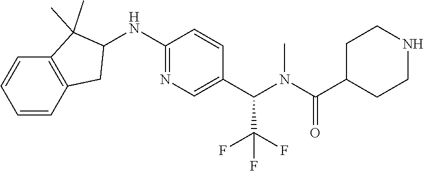 [SMILES: CN(C(=O)C1CCNCC1)[C@@H](c1ccc(NC2Cc3ccccc3C2(C)C)nc1)C(F)(F)F]

### Example 149: N-((1S)-1-(6-((1,1-Dimethyl-2,3-dihydro-1H-inden-2-yl)amino)pyridin-3-yl)-2,2,2-trifluoroethyl)-N-methylpiperidine-4-carboxamide—Isomer 2

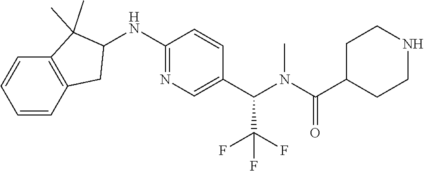 [SMILES: CN(C(=O)C1CCNCC1)[C@@H](c1ccc(NC2Cc3ccccc3C2(C)C)nc1)C(F)(F)F]

The title compounds were prepared from Intermediate 73 (0.09 g, 0.16 mmol) in 1,4-dioxane (0.45 mL) at 0° C. treated with 4M HCl in 1,4-dioxane (0.45 mL, 1.8 mmol) then stirred at RT for 1 h. The mixture was concentrated under reduced pressure then diluted with saturated aqueous NaHCO₃ and extracted with EtOAc. The organic layer was dried over Na₂SO₄, filtered and concentrated under reduced pressure. Purified by achiral preparative SFC (YMCPak Diol, 30×250 mm×5 μm, temp 30° C., Back Pressure 100 bar, flow rate: 100 g/min, 70% CO₂ with 30% 10 mM NH₄HCO₃ in MeOH modifier). Chiral purification was carried out by preparative SFC (Chiralpak®-IG, 30×250 mm×5 μm, temp 30° C., Back Pressure 100 bar, flow rate: 100 g/min, 55% CO₂ with 45% 5 mM NH₄HCO₃ in MeOH modifier) to provide Example 148 (7.4 mg) as Peak 1 and Example 149 (9.6 mg) as Peak 2. Example 148: ¹H NMR (400 MHz; DMSO-d₆) δ: 7.93 (br s, 1H), 7.36 (d, 1H), 7.22-7.12 (m, 4H), 6.90 (d, 1H), 6.67 (d, 1H), 6.38 (dd, 1H), 4.59 (dd, 1H), 3.16 (dd, 1H), 3.03-2.93 (m, 2H), 2.91 (s, 3H), 2.85-2.54 (m, 4H), 1.71-1.40 (m, 4H), 1.29 (s, 3H), 1.23 (br s, 1H), 1.08 (s, 3H). LCMS (Method A): 461.5 [M+H]⁺. Example 149: ¹H NMR (400 MHz; DMSO-d₆) δ: 7.92 (br s, 1H), 7.41-7.34 (m, 1H), 7.22-7.12 (m, 4H), 6.93-6.87 (m, 1H), 6.67 (d, 1H), 6.38 (dd, 1H), 4.58 (dd, 1H), 3.19-3.09 (m, 3H), 2.99-2.84 (m, 4H), 2.80-2.54 (m, 3H), 1.99-1.91 (m, 1H), 1.80-1.52 (m, 3H), 1.29 (s, 3H), 1.23 (br s, 1H), 1.08 (s, 3H). LCMS (Method A): 461.5 [M+H]⁺.

### Example 150: N-((1S)-1-(4-((6-Chloro-1,1-dimethyl-2,3-dihydro-1H-inden-2-yl)amino)phenyl)-2,2,2-trifluoroethyl)-N-methyltetrahydro-2H-thiopyran-4-carboxamide 1,1-dioxide—Isomer 1

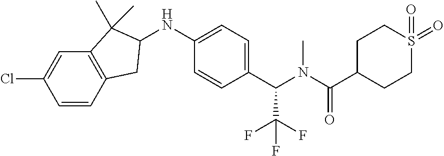 [SMILES: CN(C(=O)C1CCS(=O)(=O)CC1)[C@@H](c1ccc(NC2Cc3ccc(Cl)cc3C2(C)C)cc1)C(F)(F)F]

### Example 151: N-((1S)-1-(4-((6-Chloro-1,1-dimethyl-2,3-dihydro-1H-inden-2-yl)amino)phenyl)-2,2,2-trifluoroethyl)-N-methyltetrahydro-2H-thiopyran-4-carboxamide 1,1-dioxide—Isomer 2

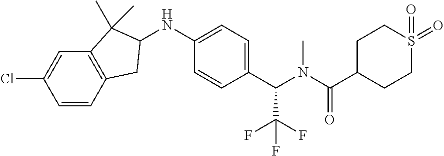 [SMILES: CN(C(=O)C1CCS(=O)(=O)CC1)[C@@H](c1ccc(NC2Cc3ccc(Cl)cc3C2(C)C)cc1)C(F)(F)F]

### Example 152: N-((1S)-1-(4-((5-Chloro-1,1-dimethyl-2,3-dihydro-1H-inden-2-yl)amino)phenyl)-2,2,2-trifluoroethyl)-N-methyltetrahydro-2H-thiopyran-4-carboxamide 1,1-dioxide—Isomer 1

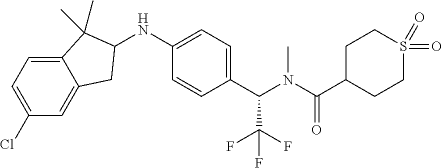 [SMILES: CN(C(=O)C1CCS(=O)(=O)CC1)[C@@H](c1ccc(NC2Cc3cc(Cl)ccc3C2(C)C)cc1)C(F)(F)F]

### Example 153: N-((1S)-1-(4-((5-Chloro-1,1-dimethyl-2,3-dihydro-1H-inden-2-yl)amino)phenyl)-2,2,2-trifluoroethyl)-N-methyltetrahydro-2H-thiopyran-4-carboxamide 1,1-dioxide—Isomer 2

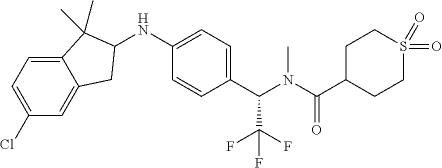 [SMILES: CN(C(=O)C1CCS(=O)(=O)CC1)[C@@H](c1ccc(NC2Cc3cc(Cl)ccc3C2(C)C)cc1)C(F)(F)F]

The title compounds were prepared in an analogous manner to Intermediate 11 from Intermediate 3 (0.40 g, 0.93 mmol), Intermediate 78 (0.22 g, 0.93 mmol), Cs₂CO₃ (1.22 g, 3.74 mmol), tBuBrettphosPdG3 (40 mg, 0.05 mmol) and RuPhos (44 mg, 0.09 mmol) in 1,4-dioxane (10 mL) at 120° C. for 2 h. Purified by flash chromatography (silica gel, eluting 50% EtOAc in petroleum ether) then by preparative HPLC (X-Select C18, 19×250 mm×5 μm, flow rate: 17 mL/min, 5 mM aqueous NH₄HCO₃ with MeCN 10% over 5 min, ramped to 50% over 0.1 min, 50% to 80% over 20 min, ramped to 95% over 0.1 min and held for 5 min). Chiral purification was carried out by preparative SFC (Chiralcel-OJ-H, 30×250 mm×5 μm, temp 30° C., Back Pressure 100 bar, flow rate: 90 g/min, 80% CO₂ with 20% MeOH/MeCN 1:1 modifier) to provide Example 150 (12 mg) as Peak 1, Example 151 (14 mg) as Peak 2, Example 152 (17 mg) as Peak 3 and Example 153 (17 mg) as Peak 4. Example 150: ¹H NMR (400 MHz; DMSO-d₆) δ: 7.27-7.11 (m, 3H), 7.05 (d, 2H), 6.78 (d, 2H), 6.35 (dd, 1H), 5.97 (d, 1H), 4.01 (dd, 1H), 3.30-3.08 (m, 6H), 2.88 (s, 3H), 2.70 (dd, 1H), 2.10-1.98 (m, 4H), 1.31 (s, 3H), 1.10 (s, 3H). LCMS (Method A): 543.3 [M+H]⁺. Example 151: ¹H NMR (400 MHz; DMSO-d₆) δ: 7.27-7.11 (m, 3H), 7.05 (d, 2H), 6.78 (d, 2H), 6.34 (dd, 1H), 5.97 (d, 1H), 4.01 (dd, 1H), 3.30-3.08 (m, 6H), 2.89 (s, 3H), 2.70 (dd, 1H), 2.10-1.98 (m, 4H), 1.31 (s, 3H), 1.10 (s, 3H). LCMS (Method A): 543.3 [M+H]⁺. Example 152: ¹H NMR (400 MHz; DMSO-d₆) δ: 7.29-7.11 (m, 3H), 7.05 (d, 2H), 6.78 (d, 2H), 6.34 (dd, 1H), 5.97 (d, 1H), 4.01 (dd, 1H), 3.30-3.08 (m, 6H), 2.89 (s, 3H), 2.73 (dd, 1H), 2.11-1.98 (m, 4H), 1.30 (s, 3H), 1.08 (s, 3H). LCMS (Method A): 543.3 [M+H]⁺. Example 153: ¹H NMR (400 MHz; DMSO-d₆) δ: 7.29-7.11 (m, 3H), 7.05 (d, 2H), 6.78 (d, 2H), 6.34 (dd, 1H), 5.97 (d, 1H), 4.01 (dd, 1H), 3.30-3.08 (m, 6H), 2.88 (s, 3H), 2.73 (dd, 1H), 2.11-1.98 (m, 4H), 1.30 (s, 3H), 1.08 (s, 3H). LCMS (Method A): 543.3 [M+H]⁺.

### Example 154: N-((1S)-1-(5-((1,1-Dimethyl-2,3-dihydro-1H-inden-2-yl)amino)pyridin-2-yl)-2,2,2-trifluoroethyl)-N-methyl-1-azaspiro[3.3]heptane-6-carboxamide—Isomer 1

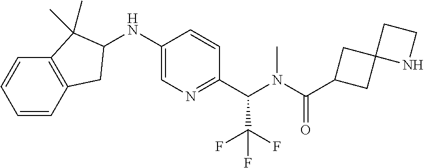 [SMILES: CN(C(=O)C1CC2(CCN2)C1)[C@@H](c1ccc(NC2Cc3ccccc3C2(C)C)cn1)C(F)(F)F]

A solution of Intermediate 79a (0.57 g, 0.99 mmol) in TFA (0.38 mL) and DCM (11.4 mL) was stirred at RT for 16 h. The mixture was concentrated under reduced pressure and triturated in pentane to provide Example 154 TFA salt (0.8 g). 100 mg of the TFA salt was purified by preparative HPLC (XBridge C18, 19×250 mm×5 μm, flow rate: 18 mL/min, 10 mM aqueous NH₄HCO₃ with MeCN 14% to 49% over 6 min, to 51% over 6 min, held at 51% for 1.5 min, ramped to 100% over 0.1 min, held for 1.4 min) to provide the title compound (6.4 mg). ¹H NMR (400 MHz; DMSO-d₆) δ: 8.13 (d, 1H), 7.22-7.04 (m, 6H), 6.35 (dd, 1H), 6.23-6.17 (m, 1H), 4.01 (dd, 1H), 3.27-3.14 (m, 3H), 2.82 (s, 3H), 2.78-2.65 (m, 2H), 2.44-2.22 (m, 3H), 2.18-2.08 (m, 2H), 1.91 (br s, 1H), 1.32 (s, 3H), 1.10 (s, 3H). LCMS (Method A): 473.5 [M+H]⁺.

### Example 155: N-((1S)-1-(5-((1,1-Dimethyl-2,3-dihydro-1H-inden-2-yl)amino)pyridin-2-yl)-2,2,2-trifluoroethyl)-N-methyl-1-azaspiro[3.3]heptane-6-carboxamide—Isomer 2

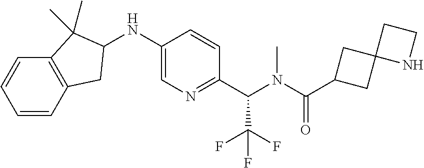 [SMILES: CN(C(=O)C1CC2(CCN2)C1)[C@@H](c1ccc(NC2Cc3ccccc3C2(C)C)cn1)C(F)(F)F]

A solution of Intermediate 79b (0.38 g, 0.66 mmol) in TFA (0.25 mL) and DCM (3.8 mL) was stirred at RT for 16 h. The mixture was concentrated under reduced pressure and triturated in pentane to provide Example 155 TFA salt (0.5 g). 100 mg of the TFA salt residue was purified by preparative HPLC (XBridge C18, 19×250 mm×5 μm, flow rate: 18 mL/min, 10 mM aqueous NH₄HCO₃ with MeCN 30% to 75% over 18 min, ramped to 100% over 0.1 min, held for 4.9 min) to provide the title compound (6.9 mg). ¹H NMR (400 MHz; DMSO-d₆) δ: 8.13 (d, 1H), 7.22-7.04 (m, 6H), 6.33 (dd, 1H), 6.23-6.18 (m, 1H), 4.01 (dd, 1H), 3.27-3.14 (m, 3H), 3.04-2.96 (m, 1H), 2.86 (s, 3H), 2.78-2.65 (m, 1H), 2.46-2.06 (m, 6H), 1.32 (s, 3H), 1.10 (s, 3H). LCMS (Method A): 473.5 [M+H]⁺.

### Example 156: (4,6-trans)-N-((1S)-1-(5-((1,1-Dimethyl-2,3-dihydro-1H-inden-2-yl)amino)pyridin-2-yl)-2,2,2-trifluoroethyl)-N,1-dimethyl-1-azaspiro[3.3]heptane-6-carboxamide—Isomer 1

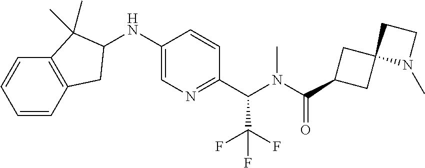 [SMILES: CN(C(=O)[C@H]1C[C@@]2(CCN2C)C1)[C@@H](c1ccc(NC2Cc3ccccc3C2(C)C)cn1)C(F)(F)F]

### Example 157: (4,6-trans)-N-((1S)-1-(5-((1,1-Dimethyl-2,3-dihydro-1H-inden-2-yl)amino)pyridin-2-yl)-2,2,2-trifluoroethyl)-N,1-dimethyl-1-azaspiro[3.3]heptane-6-carboxamide—Isomer 2

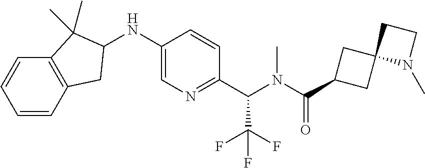 [SMILES: CN(C(=O)[C@H]1C[C@@]2(CCN2C)C1)[C@@H](c1ccc(NC2Cc3ccccc3C2(C)C)cn1)C(F)(F)F]

To a stirred mixture of Example 154 TFA salt (0.70 g, 1.19 mmol) in MeOH (14 mL) was added triethylamine (0.61 mL, 4.44 mmol) and paraformaldehyde (48 mg, 1.62 mmol) and stirred at 60° C. for 16 h. The mixture was cooled to 0° C. then sodium borohydride (0.14 g, 3.70 mmol) was added and stirred at RT for 2 h. The mixture was diluted with water and extracted with EtOAc. The organic layer was dried over Na₂SO₄, filtered and concentrated under reduced pressure. Purified by preparative HPLC (XBridge C18, 19×150 mm×5 μm, flow rate: 18 mL/min, 10 mM aqueous NH₄HCO₃ with MeCN 45% to 60% over 13 min, ramped to 98% over 0.1 min and held for 1.9 min). Chiral purification was carried out by preparative SFC (Lux Cellulose-C2, 30×250 mm×5 μm, temp 30° C., Back Pressure 100 bar, flow rate: 100 g/min, 60% CO₂ with 40% (10 mM NH₄HCO₃ in methanol) modifier to provide Example 156 (23 mg) as Peak 1 and Example 157 (33 mg) as Peak 2. Example 156: ¹H NMR (400 MHz; DMSO-d₆) δ: ¹H NMR (400 MHz; DMSO-d₆) δ: 8.14 (d, 1H), 7.22-7.05 (m, 6H), 6.33 (dd, 1H), 6.22-6.17 (m, 1H), 4.02 (dd, 1H), 3.25-3.16 (m, 2H), 3.08-2.99 (m, 2H), 2.83 (s, 3H), 2.78-2.56 (m, 2H), 2.34-1.98 (m, 8H), 1.32 (s, 3H), 1.10 (s, 3H). LCMS (Method I): 487.4 [M+H]⁺. Example 157: ¹H NMR (400 MHz; DMSO-d₆) δ: 8.14 (d, 1H), 7.22-7.05 (m, 6H), 6.37 (dd, 1H), 6.22-6.17 (m, 1H), 4.04 (dd, 1H), 3.25-3.14 (m, 2H), 2.96 (t, 2H), 2.82 (s, 3H), 2.78-2.46 (m, 3H), 2.19-2.08 (m, 5H), 2.03-1.92 (m, 2H), 1.32 (s, 3H), 1.10 (s, 3H). LCMS (Method I): 487.4 [M+H]⁺.

### Example 158: (4,6-cis)-N-((1S)-1-(5-((1,1-Dimethyl-2,3-dihydro-1H-inden-2-yl)amino)pyridin-2-yl)-2,2,2-trifluoroethyl)-N,1-dimethyl-1-azaspiro[3.3]heptane-6-carboxamide—Isomer 1

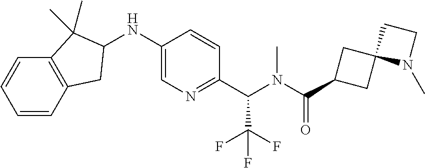 [SMILES: CN(C(=O)[C@H]1C[C@]2(CCN2C)C1)[C@@H](c1ccc(NC2Cc3ccccc3C2(C)C)cn1)C(F)(F)F]

### Example 159: (4,6-cis)-N-((1S)-1-(5-((1,1-Dimethyl-2,3-dihydro-1H-inden-2-yl)amino)pyridin-2-yl)-2,2,2-trifluoroethyl)-N,1-dimethyl-1-azaspiro[3.3]heptane-6-carboxamide—Isomer 2

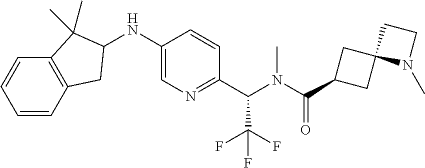 [SMILES: CN(C(=O)[C@H]1C[C@]2(CCN2C)C1)[C@@H](c1ccc(NC2Cc3ccccc3C2(C)C)cn1)C(F)(F)F]

To a stirred mixture of Example 155 TFA salt (0.40 g, 0.68 mmol) in MeOH (8 mL) was added triethylamine (0.35 mL, 2.54 mmol) and paraformaldehyde (27 mg, 0.93 mmol) and stirred at 60° C. for 16 h. The mixture was cooled to 0° C. then sodium borohydride (80 mg, 2.11 mmol) was added and stirred at RT for 2 h. The mixture was diluted with water and extracted with EtOAc. The organic layer was dried over Na₂SO₄, filtered and concentrated under reduced pressure. Purified by preparative HPLC (XBridge C18, 19×150 mm×5 μm, flow rate: 18 mL/min, 10 mM aqueous NH₄HCO₃ with MeCN 30% to 50% over 3 min, to 68 over 5 min, held at 68\hat{ } for 3.5 min, ramped to 10% over 0.1 min and held for 3.4 min). Chiral purification was carried out by preparative SFC (Lux Cellulose-C2, 30×250 mm×5 μm, temp 30° C., Back Pressure 100 bar, flow rate: 100 g/min, 60% CO₂ with 40% (5 mM NH₄HCO₃ in methanol) modifier to provide Example 158 (31 mg) as Peak 1 and Example 159 (31 mg) as Peak 2. Example 158: ¹H NMR (400 MHz; DMSO-d₆) δ: ¹H NMR (400 MHz; DMSO-d₆) δ: 8.14 (d, 1H), 7.22-7.05 (m, 6H), 6.33 (dd, 1H), 6.22-6.17 (m, 1H), 4.02 (dd, 1H), 3.20 (dd, 1H), 3.09-3.02 (m, 1H), 2.97 (t, 2H), 2.89 (s, 3H), 2.78-2.70 (m, 1H), 2.52 (br s, 1H), 2.30-2.04 (m, 8H), 1.32 (s, 3H), 1.10 (s, 3H). LCMS (Method I): 487.4 [M+H]⁺. Example 159: ¹H NMR (400 MHz; DMSO-d₆) δ: 8.13 (d, 1H), 7.22-7.05 (m, 6H), 6.33 (dd, 1H), 6.22-6.17 (m, 1H), 4.02 (dd, 1H), 3.20 (dd, 1H), 3.09-3.02 (m, 1H), 2.98 (t, 2H), 2.88 (s, 3H), 2.78-2.70 (m, 1H), 2.54-2.50 (m, 1H), 2.30-2.06 (m, 8H), 1.32 (s, 3H), 1.10 (s, 3H). LCMS (Method I): 487.4 [M+H]⁺.

### Example 160: (S)—N—((S)-1-(5-(((S)-1,1-Dimethyl-2,3-dihydro-1H-inden-2-yl)amino)pyridin-2-yl)-2,2,2-trifluoroethyl)-N-methyl-5-oxopyrrolidine-3-carboxamide

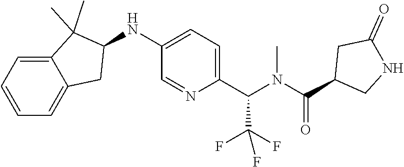 [SMILES: CN(C(=O)[C@@H]1CNC(=O)C1)[C@@H](c1ccc(N[C@H]2Cc3ccccc3C2(C)C)cn1)C(F)(F)F]

The title compound (97 mg) was prepared in an analogous manner to Intermediate 3 from Intermediate 82 (0.15 g, 0.43 mmol), pyridine (1.5 mL), T3P® (50% EtOAc, 0.79 mL, 1.29 mmol) and (3S)-5-oxopyrrolidine-3-carboxylic acid (83 mg, 0.64 mmol, CAS 30948-17-5) at RT for 16 h. Purified by preparative HPLC (Gemini-NX-C18, 21×250 mm×5 μm, flow rate: 35 mL/min, 0.05% aqueous ammonia with MeCN 45% isocratic for 12 min, ramped to 95% over 0.1 min, held for 1.8 min). ¹H NMR (400 MHz; DMSO-d₆) δ: 8.26-8.11 (m, 1H), 7.34 (s, 1H), 7.28-7.07 (m, 6H), 6.39 (s, 1H), 5.93 (d, 1H), 4.05 (dd, 1H), 3.75 (s, 1H), 3.57 (dd, 1H), 3.33 (dd, 1H), 3.25 (dd, 1H), 3.01 (d, 3H), 2.78 (dd, 1H), 2.39 (d, 2H), 1.35 (s, 3H), 1.15 (s, 3H). LCMS (Method N): 461.3 [M+H]⁺.

### Example 161: N-((1S)-1-(5-((1,1-Dimethyl-2,3-dihydro-1H-inden-2-yl)amino)pyridin-2-yl)-2,2,2-trifluoroethyl)-N-methyl-5-oxomorpholine-3-carboxamide—Isomer 1

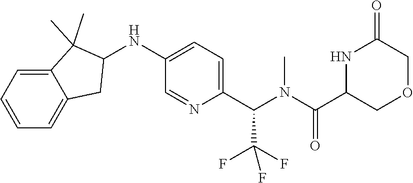 [SMILES: CN(C(=O)C1COCC(=O)N1)[C@@H](c1ccc(NC2Cc3ccccc3C2(C)C)cn1)C(F)(F)F]

### Example 162: N-((1S)-1-(5-((1,1-Dimethyl-2,3-dihydro-1H-inden-2-yl)amino)pyridin-2-yl)-2,2,2-trifluoroethyl)-N-methyl-5-oxomorpholine-3-carboxamide—Isomer 2

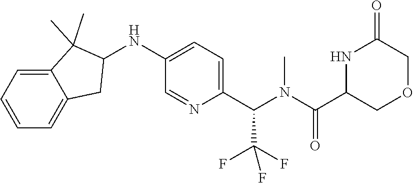 [SMILES: CN(C(=O)C1COCC(=O)N1)[C@@H](c1ccc(NC2Cc3ccccc3C2(C)C)cn1)C(F)(F)F]

### Example 163: N-((1S)-1-(5-((1,1-Dimethyl-2,3-dihydro-1H-inden-2-yl)amino)pyridin-2-yl)-2,2,2-trifluoroethyl)-N-methyl-5-oxomorpholine-3-carboxamide—Isomer 3

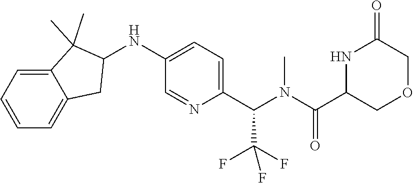 [SMILES: CN(C(=O)C1COCC(=O)N1)[C@@H](c1ccc(NC2Cc3ccccc3C2(C)C)cn1)C(F)(F)F]

### Example 164: N-((1S)-1-(5-((1,1-Dimethyl-2,3-dihydro-1H-inden-2-yl)amino)pyridin-2-yl)-2,2,2-trifluoroethyl)-N-methyl-5-oxomorpholine-3-carboxamide—Isomer 4

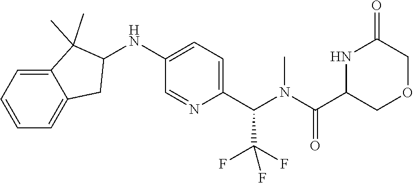 [SMILES: CN(C(=O)C1COCC(=O)N1)[C@@H](c1ccc(NC2Cc3ccccc3C2(C)C)cn1)C(F)(F)F]

The title compounds were prepared in an analogous manner to Intermediate 3 from Intermediate 61 (0.50 g, 0.23 mmol), pyridine (2.5 mL), T3P® (50% EtOAc, 4.6 mL, 15.5 mmol) and 5-oxomorpholine-3-carboxylic acid (0.57 g, 3.86 mmol, CAS 1367717-62-1) at RT for 16 h. Purified purified by reverse phase chromatography (40 g C18, eluting 25% MeCN in 0.1M NH₄HCO₃). Chiral purification was carried out by preparative SFC (Chiralpak®-AS-H, 30×250 mm×5 μm, temp 30° C., Back Pressure 100 bar, flow rate: 100 g/min, 60% CO₂ with 40% MeOH modifier); (Chiralpak®-AD-H, 30×250 mm×5 μm, temp 30° C., Back Pressure 100 bar, flow rate: 110 g/min, 55% CO₂ with 45% 1:1 MeOH/MeCN modifier) and (Chiralpak®-IG, 30×250 mm×5 μm, temp 30° C., Back Pressure 100 bar, flow rate: 110 g/min, 55% CO₂ with 45% 1:1 MeOH/MeCN modifier) to provide Example 161 (9 mg) as Peak 1, Example 162 (9 mg) as Peak 2, Example 163 (12 mg) as Peak 3 and Example 164 (12 mg) as Peak 4. Example 161: ¹H NMR (400 MHz; DMSO-d₆) δ: 8.21-8.14 (m, 2H), 7.20-7.07 (m, 6H), 6.35-6.05 (m, 2H), 4.65 (m, 1H), 4.07-3.88 (m, 4H), 3.79-3.76 (m, 1H), 3.22-3.17 (m, 1H), 2.99 (s, 3H) 2.82-2.67 (m, 1H), 1.32 (s, 3H), 1.10 (s, 3H). LCMS (Method A): 477.3 [M+H]⁺. Example 162: ¹H NMR (400 MHz; DMSO-d₆) δ: 8.21-8.14 (m, 2H), 7.27-7.07 (m, 6H), 6.35-6.05 (m, 2H), 4.95-4.65 (m, 1H), 4.07-3.91 (m, 4H), 3.89-3.76 (m, 1H), 3.22-3.16 (m, 1H), 2.99 (s, 3H) 2.81-2.71 (m, 1H), 1.32 (s, 3H), 1.10 (s, 3H). LCMS (Method A): 477.3 [M+H]⁺. Example 163: ¹H NMR (400 MHz; DMSO-d₆) δ: 8.22-7.97 (m, 2H), 7.22-7.07 (m, 6H), 6.39-5.99 (m, 2H), 4.84-4.65 (m, 1H), 4.07-3.94 (m, 4H), 3.85-3.82 (m, 1H) 3.22-3.17 (m, 1H), 3.02 (s, 3H) 2.78-2.72 (m, 1H), 1.32 (s, 3H), 1.10 (s, 3H). LCMS (Method A): 477.3 [M+H]⁺. Example 164: ¹H NMR (400 MHz; DMSO-d₆) δ: 8.22-7.98 (m, 2H), 7.24-7.11 (m, 6H), 6.39-5.99 (m, 2H), 4.84-4.65 (m, 1H), 4.07-3.94 (m, 4H), 3.85-3.81 (m, 1H), 3.22-3.16 (m, 1H), 3.02 (s, 3H) 2.77-2.71 (m, 1H), 1.32 (s, 3H), 1.13 (s, 3H). LCMS (Method A): 477.3 [M+H]⁺.

### Example 165: (S)—N—((S)-1-(5-(((S)-1,1-Dimethyl-2,3-dihydro-1H-inden-2-yl)amino)pyridin-2-yl)-2,2,2-trifluoroethyl)-N-methyl-4-oxoazetidine-2-carboxamide

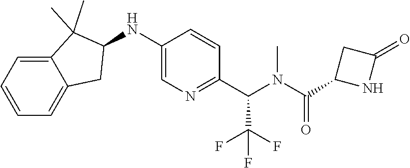 [SMILES: CN(C(=O)[C@@H]1CC(=O)N1)[C@@H](c1ccc(N[C@H]2Cc3ccccc3C2(C)C)cn1)C(F)(F)F]

The title compound (88 mg) was prepared in an analogous manner to Intermediate 3 from Intermediate 82 (0.15 g, 0.43 mmol), pyridine (1.4 mL), T3P® (50% MeTHF, 2.6 mL, 1.30 mmol) and (2S)-4-oxoazetidine-2-carboxylic acid (83 mg, 0.64 mmol, CAS 16404-97-7) at RT for 16 h. Purified by preparative HPLC (Gemini-NX-C18, 21×250 mm×5 μm, flow rate: 30 mL/min, 0.1% aqueous formic acid with MeCN 50% to 69% over 3 min, held for 1 min, ramped to 98% over 0.1 min, held for 2.0 min). ¹H NMR (400 MHz; DMSO-d₆) δ: 8.36 (s, 1H), 8.16 (m, 1H), 7.28-7.05 (m, 6H), 6.33 (m, 1H), 6.26 (d, 1H), 4.54 (dd, 1H), 4.06 (dd, 1H), 3.31-3.28 (m, 1H), 3.20 (dd, 1H), 2.93 (s, 3H), 2.82-2.71 (m, 2H), 1.33 (s, 3H), 1.11 (s, 3H). LCMS (Method M): 447.3 [M+H]⁺.

### Example 166: (R)—N—((S)-1-(5-(((S)-1,1-Dimethyl-2,3-dihydro-1H-inden-2-yl)amino)pyridin-2-yl)-2,2,2-trifluoroethyl)-N-methyl-5-oxopyrrolidine-3-carboxamide

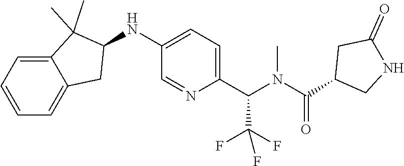 [SMILES: CN(C(=O)[C@H]1CNC(=O)C1)[C@@H](c1ccc(N[C@H]2Cc3ccccc3C2(C)C)cn1)C(F)(F)F]

The title compound (44 mg) was prepared in an analogous manner to Intermediate 3 from Intermediate 82 (0.15 g, 0.43 mmol), pyridine (1.4 mL), T3P® (50% MeTHF, 2.6 mL, 1.30 mmol) and (3R)-5-oxopyrrolidine-3-carboxylic acid (83 mg, 0.64 mmol, CAS 428518-37-0) at RT for 16 h. Purified by preparative HPLC (Gemini-NX-C18, 21×250 mm×5 μm, flow rate: 35 mL/min, 0.05% aqueous ammonia with MeCN 40% to 50% over 11 min, ramped to 95% over 0.2 min, held for 1.8 min). ¹H NMR (300 MHz; DMSO-d₆) δ: 8.23-8.09 (m, 1H), 7.66 (s, 1H), 7.31-7.07 (m, 6H), 6.37 (dd, 1H), 6.23 (d, 1H), 4.05 (dd, 1H), 3.83-3.62 (m, 1H), 3.52-3.41 (m, 1H), 3.30-3.26 (m, 1H), 3.20 (dd, 1H), 2.97 (s, 3H), 2.82-2.68 (m, 2H), 2.34-2.22 (m, 1H), 1.33 (s, 3H), 1.11 (s, 3H). LCMS (Method M): 461.1 [M+H]⁺.

### Example 167: (S)—N—((S)-1-(5-(((S)-1,1-Dimethyl-2,3-dihydro-1H-inden-2-yl)amino)pyridin-2-yl)-2,2,2-trifluoroethyl)-N-methylmorpholine-2-carboxamide dihydrochloride

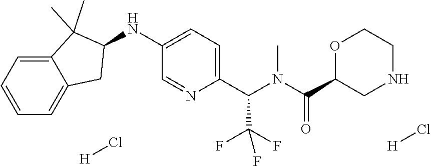 [SMILES: CN(C(=O)[C@@H]1CNCCO1)[C@@H](c1ccc(N[C@H]2Cc3ccccc3C2(C)C)cn1)C(F)(F)F ; Cl ; Cl]

To a stirred solution of Intermediate 83 (0.21 g, 0.37 mmol) in 1,4-dioxane (1.5 mL) was added 4M HCl in 1,4-dioxane (1.4 mL) at RT for 16 h. The mixture was concentrated under reduced pressure and the residue was triturated in diethyl ether to provide the title compound (0.17 g). ¹H NMR (300 MHz; DMSO-d₆) δ: 8.09 (d, 1H), 7.24-7.06 (m, 6H), 6.21 (dd, 1H), 4.68 (dd, 1H), 4.00 (d, 1H), 3.79 (dd, 1H), 3.35-3.03 (m, 5H), 3.01 (s, 3H), 2.71 (dd, 1H), 2.53-2.50 (m, 1H), 1.28 (s, 3H), 1.04 (s, 3H)—4 NH protons not observed. LCMS (Method M): 463.3 [M+H]⁺.

### Example 168: (S)—N—((S)-1-(5-(((S)-1,1-Dimethyl-2,3-dihydro-1H-inden-2-yl)amino)pyridin-2-yl)-2,2,2-trifluoroethyl)-N-methylpyrrolidine-3-carboxamide

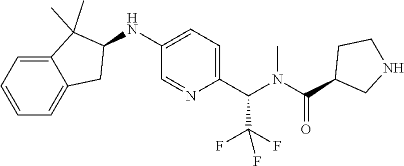 [SMILES: CN(C(=O)[C@H]1CCNC1)[C@@H](c1ccc(N[C@H]2Cc3ccccc3C2(C)C)cn1)C(F)(F)F]

To a stirred solution of Intermediate 84 (0.18 g, 0.33 mmol) in 1,4-dioxane (2.2 mL) was added 4M HCl in 1,4-dioxane (0.49 mL) at RT for 16 h. The mixture was cooled and basified with 1M aqueous NaOH then extracted with DCM. The organic layer washed with brine, dried over Na₂SO₄, filtered and concentrated under reduced pressure. The crude product was purified by preparative HPLC (Gemini-NX-C18, 21×250 mm×5 μm, flow rate: 30 mL/min, 0.1% aqueous formic acid with MeCN 55% to 60% over 6 min, held for 5 min ramped to 95% over 0.2 min, held for 1.8 min) to provide the title compound (0.16 g). ¹H NMR (400 MHz; DMSO-d₆) δ: 8.16 (d, 1H), 7.23-7.19 (m, 3H), 7.19-7.07 (m, 3H), 6.38 (dd, 1H), 6.24 (d, 1H), 4.05 (dd, 1H), 3.40-3.33 (m, 3H), 3.24-3.16 (m, 3H), 3.00 (s, 3H), 2.97-2.91 (m, 1H), 2.79-2.71 (m, 1H), 2.05-1.95 (m, 1H), 1.86-1.75 (m, 1H), 1.33 (s, 3H), 1.11 (s, 3H). LCMS (Method K): 447.1 [M+H]⁺.

### Example 169: (S)-1-Acetyl-N—((S)-1-(5-(((S)-1,1-dimethyl-2,3-dihydro-1H-inden-2-yl)amino)pyridin-2-yl)-2,2,2-trifluoroethyl)-N-methylpyrrolidine-3-carboxamide

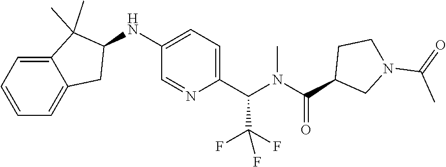 [SMILES: CC(=O)N1CC[C@H](C(=O)N(C)[C@@H](c2ccc(N[C@H]3Cc4ccccc4C3(C)C)cn2)C(F)(F)F)C1]

To a stirred mixture of Example 168 (0.11 g, 0.24 mmol) and triethylamine (0.10 mL, 0.72 mmol) in DCM (2 mL) was added acetyl chloride (1M in DCM, 0.24 mL, 0.24 mmol) and the mixture was stirred at RT for 15 min. The mixture was poured into water then extracted with DCM. The organic layer was dried over Na₂SO₄, filtered and concentrated under reduced pressure. The crude product was purified by preparative HPLC (Gemini-NX-C18, 21×250 mm×5 μm, flow rate: 30 mL/min, 0.1% aqueous formic acid with 0.1% formic acid in MeCN; 50% isocratic for 11 min, ramped to 95% over 0.2 min, held for 2.0 min) to provide the title compound (41 mg). ¹H NMR (400 MHz; DMSO-d₆) δ: 8.19-8.14 (m, 1H), 7.23-7.09 (m, 5H), 6.42-6.33 (m, 1H), 6.24 (d, 1H), 4.06 (dd, 1H), 3.79-3.72 (m, 1H), 3.66-3.38 (m, 4H), 3.20 (dd, 1H), 3.03 (d, 3H), 2.80-2.70 (m, 2H), 2.18-1.99 (m, 2H), 1.95 (d, 3H), 1.33 (s, 3H), 1.11 (s, 3H). LCMS (Method M): 489.2 [M+H]⁺.

### Example 170: (S)—N—((S)-1-(5-(((S)-1,1-Dimethyl-2,3-dihydro-1H-inden-2-yl)amino)pyridin-2-yl)-2,2,2-trifluoroethyl)-N-methyl-5-oxopyrrolidine-2-carboxamide

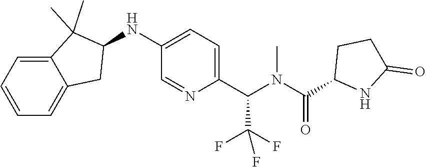 [SMILES: CN(C(=O)[C@@H]1CCC(=O)N1)[C@@H](c1ccc(N[C@H]2Cc3ccccc3C2(C)C)cn1)C(F)(F)F]

The title compound (11 mg) was prepared in an analogous manner to Intermediate 3 from Intermediate 82 (0.10 g, 0.29 mmol), pyridine (1.4 mL), T3P® (50% MeTHF, 0.53 mL, 0.87 mmol) and (2S)-5-oxopyrrolidine-2-carboxylic acid (55 mg, 0.43 mmol, CAS 98-79-3) at RT for 16 h. Purified by preparative HPLC (Gemini-NX-C18, 21×250 mm×5 μm, flow rate: 30 mL/min, 0.1% aqueous formic acid with MeCN 30-40% over 6 min, held at 40% for 18 min, ramped to 95% over 0.1 min, held for 1.8 min). ¹H NMR (400 MHz; DMSO-d₆) δ: 8.17 (d, 1H), 7.81 (s, 1H), 7.25-7.09 (m, 6H), 6.36 (dd, 1H), 6.25 (d, 1H), 4.64 (dd, 1H), 4.06 (dd, 1H), 3.24-3.15 (m, 1H), 3.01 (s, 3H), 2.80-2.70 (m, 1H), 2.47-2.37 (m, 1H), 2.19-2.07 (m, 2H), 1.89-1.79 (m, 1H), 1.33 (s, 3H), 1.11 (s, 3H). LCMS (Method M): 461.3 [M+H]⁺.

### Example 171: (R)-1-Acetyl-N—((S)-1-(5-(((S)-1,1-dimethyl-2,3-dihydro-1H-inden-2-yl)amino)pyridin-2-yl)-2,2,2-trifluoroethyl)-N-methylpyrrolidine-2-carboxamide

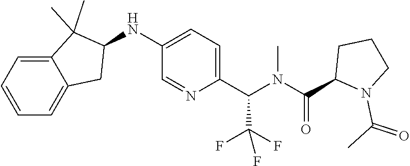 [SMILES: CC(=O)N1CCC[C@@H]1C(=O)N(C)[C@@H](c1ccc(N[C@H]2Cc3ccccc3C2(C)C)cn1)C(F)(F)F]

The title compound (26 mg) was prepared in an analogous manner to Intermediate 3 from Intermediate 82 (50 mg, 0.14 mmol), pyridine (2.1 mL), T3P® (50% MeTHF, 0.27 mL, 0.43 mmol) and (2R)-1-acetylpyrrolidine-2-carboxylic acid (55 mg, 0.43 mmol, CAS 59785-68-1) at RT for 16 h. Purified by preparative HPLC (Gemini-NX-C18, 21×250 mm×5 μm, flow rate: 30 mL/min, 0.05% aqueous ammonia with MeCN 50% isocratic for 20 min). ¹H NMR (400 MHz; DMSO-d₆) δ: 8.16-8.11 (m, 1H), 7.28-7.04 (m, 6H), 6.29-6.13 (m, 2H), 4.15-3.98 (m, 1H), 3.64-3.49 (m, 1H), 3.48-3.37 (m, 1H), 3.27-3.13 (m, 1H), 3.01 (s, 3H), 2.81-2.71 (m, 1H), 2.59 (s, 1H), 2.23-2.03 (m, 1H), 1.98 (d, 2H), 1.96-1.85 (m, 1H), 1.78 (s, 3H), 1.32 (s, 3H), 1.10 (s, 3H). LCMS (Method N): 489.3 [M+H]⁺.

### Example 172: (1R,3S)-3-Acetamido-N—((S)-1-(5-(((S)-1,1-dimethyl-2,3-dihydro-1H-inden-2-yl)amino)pyridin-2-yl)-2,2,2-trifluoroethyl)-N-methylcyclopentane-1-carboxamide

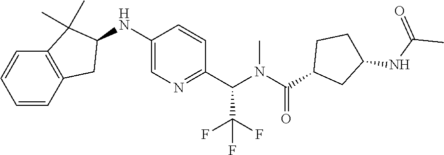 [SMILES: CC(=O)N[C@H]1CC[C@@H](C(=O)N(C)[C@@H](c2ccc(N[C@H]3Cc4ccccc4C3(C)C)cn2)C(F)(F)F)C1]

To a stirred mixture of Intermediate 86 (0.13 g, 0.29 mmol) and triethylamine (0.15 mL, 1.10 mmol) in DCM (1.2 mL) was added acetyl chloride (1M in DCM, 0.55 mL, 0.55 mmol) and the mixture was stirred at RT for 1 h. The mixture was concentrated under reduced pressure and purified by prep-TLC (silica gel, eluting 30% EtOAc in hexane) to provide the title compound (59 mg). ¹H NMR (300 MHz; DMSO-d₆) δ: 8.15 (d, 1H), 7.89 (d, 1H), 7.30-7.04 (m, 6H), 6.40 (dd, 1H), 6.22 (d, 1H), 4.05 (dd, 2H), 3.18 (m, 2H), 2.96 (s, 3H), 2.75 (m, 2H), 2.12-2.01 (m, 1H), 1.89 (d, 2H), 1.77 (s, 3H), 1.68-1.51 (m, 1H), 1.46 (d, 1H), 1.33 (s, 3H), 1.10 (s, 3H). LCMS (Method M): 503.2 [M+H]⁺.

### Example 173: N—((S)-1-(5-(((S)-1,1-Dimethyl-2,3-dihydro-1H-inden-2-yl)amino)pyridin-2-yl)-2,2,2-trifluoroethyl)-N-methyl-2-azaspiro[3.3]heptane-6-carboxamide

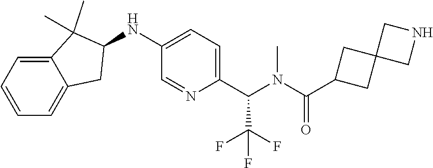 [SMILES: CN(C(=O)C1CC2(CNC2)C1)[C@@H](c1ccc(N[C@H]2Cc3ccccc3C2(C)C)cn1)C(F)(F)F]

A solution of Intermediate 87 (0.38 g, 0.66 mmol) in TFA (0.27 mL) and DCM (1.7 mL) was stirred at RT for 4 h. The mixture was poured into aqueous K₂CO₃ and extracted with DCM. The organic layer washed with brine, dried over Na₂SO₄, filtered and concentrated under reduced pressure. The crude product was purified by preparative HPLC (Kinetex XB-C18, 21×250 mm×5 μm, flow rate: 30 mL/min, 0.1% aqueous formic acid with MeCN 5% to 80% over 10 min, ramped to 95% over 0.2 min, held for 1.8 min) to provide the title compound (15 mg). ¹H NMR (400 MHz; DMSO-d₆) δ: 8.14 (s, 1H), 7.23-7.06 (m, 6H), 6.38-6.15 (m, 2H), 4.04 (dd, 1H), 3.98-3.84 (m, 2H), 3.75 (br s, 2H), 3.31 (m, 1H), 3.18 (dd, 1H), 2.83 (s, 3H), 2.80-2.69 (m, 1H), 2.54-2.23 (m, 5H), 1.32 (s, 3H), 1.09 (s, 3H). LCMS (Method M): 473.2 [M+H]⁺.

### Example 174: N-((1S)-1-(6-((1,1-Dimethyl-2,3-dihydro-1H-inden-2-yl)amino)pyridin-3-yl)-2,2,2-trifluoroethyl)-N-methyl-7-oxo-6-oxa-8-azaspiro[3.5]nonane-2-carboxamide Isomer 1

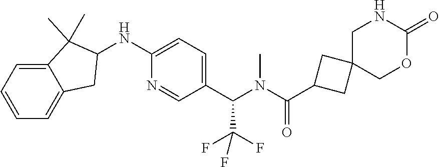 [SMILES: CN(C(=O)C1CC2(CNC(=O)OC2)C1)[C@@H](c1ccc(NC2Cc3ccccc3C2(C)C)nc1)C(F)(F)F]

### Example 175: N-((1S)-1-(6-((1,1-Dimethyl-2,3-dihydro-1H-inden-2-yl)amino)pyridin-3-yl)-2,2,2-trifluoroethyl)-N-methyl-7-oxo-6-oxa-8-azaspiro[3.5]nonane-2-carboxamide Isomer 2

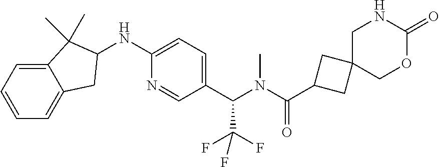 [SMILES: CN(C(=O)C1CC2(CNC(=O)OC2)C1)[C@@H](c1ccc(NC2Cc3ccccc3C2(C)C)nc1)C(F)(F)F]

A solution of Intermediate 88 (0.40 g, 0.70 mmol) in TFA (0.42 mL) and DCM (70 mL) was stirred at 40° C. for 16 h. The mixture was concentrated under reduced pressure. The crude product was purified by preparative HPLC (Gemini NX-C18, 21×250 mm×5 μm, flow rate: 30 mL/min, 0.1% aqueous formic acid with MeCN 20% to 35% over 6 min, held at 35% for 5 min, ramped to 95% over 0.2 min, held for 1.8 min) to provide 68 mg of the diastereomeric mixture. Chiral purification was carried out by preparative SFC (Chiralpak®-AD-H, 30×250 mm×5 μm, temp 30° C., Back Pressure 100 bar, flow rate: 100 g/min, 60% CO₂ with 40% MeOH/MeCN 1:1 modifier) to provide Example 174 (15 mg) as Peak 1 and Example 175 (20 mg) as Peak 2. Example 174: ¹H NMR (400 MHz; DMSO-d₆) δ: 8.00-7.95 (m, 1H), 7.42-7.37 (m, 1H), 7.25-7.12 (m, 5H), 6.95-6.90 (m, 1H), 6.70-6.66 (m, 1H), 6.38-5.66 (m, 1H), 4.59 (dd, 1H), 3.98 (s, 2H), 3.79-3.53 (m, 1H), 3.28-3.13 (m, 3H), 2.78-2.70 (m, 4H), 2.20-2.02 (m, 4H), 1.28 (s, 3H), 1.08 (s, 3H). LCMS (Method H): 517.4 [M+H]⁺. Example 175: ¹H NMR (400 MHz; DMSO-d₆) δ: 8.00-7.93 (m, 1H), 7.43-7.38 (m, 1H), 7.24-7.12 (m, 5H), 6.96-6.90 (m, 1H), 6.70-6.66 (m, 1H), 6.38-5.64 (m, 1H), 4.58 (dd, 1H), 3.98 (s, 2H), 3.79-3.55 (m, 1H), 3.28-3.13 (m, 3H), 2.77-2.70 (m, 4H), 2.18-2.03 (m, 4H), 1.29 (s, 3H), 1.08 (s, 3H). LCMS (Method H): 517.4 [M+H]⁺.

### Example 176: (R)—N—((S)-1-(5-(((S)-1,1-Dimethyl-2,3-dihydro-1H-inden-2-yl)amino)pyridin-2-yl)-2,2,2-trifluoroethyl)-N-methylpyrrolidine-3-carboxamide

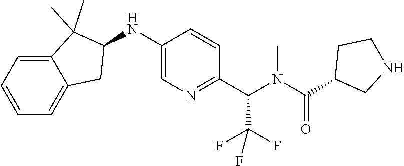 [SMILES: CN(C(=O)[C@@H]1CCNC1)[C@@H](c1ccc(N[C@H]2Cc3ccccc3C2(C)C)cn1)C(F)(F)F]

To a stirred solution of Intermediate 89 (0.21 g, 0.38 mmol) in 1,4-dioxane (2.6 mL) was added 4M HCl in 1,4-dioxane (1.15 mL) at RT for 48 h. The mixture was cooled and basified with 1M aqueous NaOH then extracted with DCM. The organic layer washed with brine, dried over Na₂SO₄, filtered and concentrated under reduced pressure. The crude product was purified by automated column chromatography (silica gel 12 g, eluting 0-10% MeOH in DCM) and triturated in pentane, to provide the title compound (0.17 g). ¹H NMR (300 MHz; MeCN-d₃) δ: 8.11 (s, 1H), 7.36-7.02 (m, 6H), 6.46 (dd, 1H), 4.88 (d, 1H), 4.07 (dd, 1H), 3.57-2.61 (m, 10H), 2.13 (dd, 2H), 1.91-1.80 (m, 1H), 1.36 (s, 3H), 1.15 (s, 3H). LCMS (Method M): 447.3 [M+H]⁺.

### Example 177: N-Methyl-N-((1S)-2,2,2-trifluoro-1-(5-((4-methoxy-2,3-dihydro-1H-inden-2-yl)amino)pyridin-2-yl)ethyl)tetrahydro-2H-thiopyran-4-carboxamide 1,1-dioxide—Isomer 1

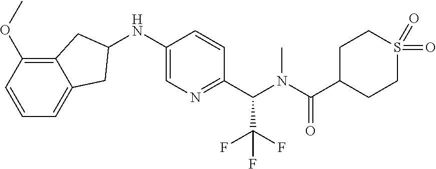 [SMILES: COc1cccc2c1CC(Nc1ccc([C@H](N(C)C(=O)C3CCS(=O)(=O)CC3)C(F)(F)F)nc1)C2]

### Example 178: N-Methyl-N-((1S)-2,2,2-trifluoro-1-(5-((4-methoxy-2,3-dihydro-1H-inden-2-yl)amino)pyridin-2-yl)ethyl)tetrahydro-2H-thiopyran-4-carboxamide 1,1-dioxide—Isomer 2

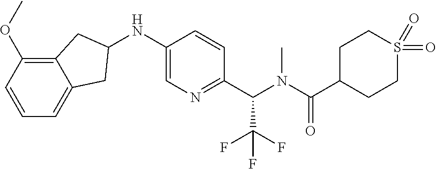 [SMILES: COc1cccc2c1CC(Nc1ccc([C@H](N(C)C(=O)C3CCS(=O)(=O)CC3)C(F)(F)F)nc1)C2]

The title compound was prepared in an analogous manner to Intermediate 11 from Intermediate 90 (0.45 g, 0.71 mmol), 4-methoxy-2,3-dihydro-1H-inden-2-amine (0.12 g, 0.71 mmol, CAS 76413-92-8), Cs₂CO₃ (0.70 g, 2.14 mmol), and RuPhosPdG3 (0.12 g, 0.14 mmol) in toluene (10 mL) at 110° C. for 2 h. Purified by preparative HPLC (XBridge C18, 30×150 mm×5 μm, flow rate: 18 mL/min, 10 mM aqueous NH₄HCO₃ with MeCN 30% to 65% over 10 min, ramped to 98% over 0.1 min and held for 1.9 min). Chiral purification was carried out by preparative SFC (LUX-1 Amylose-3, 30×250 mm×5 μm, temp 30° C., Back Pressure 100 bar, flow rate: 100 g/min, 55% CO₂ with 45% MeCN/MeOH 1:1 modifier) to provide Example 177 (18 mg) as Peak 1 and Example 178 (15 mg) as Peak 2. Example 177: ¹H NMR (400 MHz; DMSO-d₆) δ: 8.01 (d, 1H), 7.26-7.00 (m, 3H), 6.84 (d, 1H), 6.78 (d, 1H), 6.52-6.45 (m, 1H), 6.34-6.36 (m, 1H), 4.25 (t, 1H), 3.76 (s, 3H), 3.16-3.18 (m, 7H), 2.68-2.72 (m, 5H), 1.95-1.99 (m, 4H). LCMS (Method A): 512.3 [M+H]⁺. Example 178: ¹H NMR (400 MHz; DMSO-d₆) δ: 8.01 (d, 1H), 7.26-7.12 (m, 2H), 7.07-7.00 (m, 1H), 6.85 (d, 1H), 6.79 (d, 1H), 6.52-6.46 (m, 1H), 6.04-6.34 (m, 1H), 4.25-4.26 (m, 1H), 3.76 (s, 3H), 3.25-3.08 (m, 7H), 3.01 (s, 3H), 2.82-2.73 (m, 2H), 2.10-1.99 (m, 4H). LCMS (Method A): 512.3 [M+H]⁺.

### Example 179: 3-Acetamido-N—((S)-1-(5-(((S)-1,1-dimethyl-2,3-dihydro-1H-inden-2-yl)amino)pyridin-2-yl)-2,2,2-trifluoroethyl)-N-methylbicyclo[1.1.1]pentane-1-carboxamide

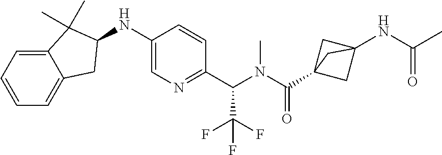

To a stirred mixture of Intermediate 92 (0.11 g, 0.20 mmol) and triethylamine (0.09 mL, 0.63 mmol) in DCE (1.6 mL) was added acetyl chloride (1M in DCM, 0.25 mL, 0.25 mmol) and the mixture was stirred at RT for 15 min. The mixture was poured into water and extracted with DCM. The organic layer washed with brine, dried over Na₂SO₄, filtered and concentrated under reduced pressure. The crude was purified by preparative HPLC (Gemini-NX C18, 21×250 mm×5 μm, flow rate: 30 mL/min, 0.1% aqueous formic acid with MeCN 40% to 65% over 6 min, held for 4 min, ramped to 95% over 0.2 min and held for 1.8 min) to provide the title compound (50 mg). ¹H NMR (400 MHz; DMSO-d₆) δ: 8.54-8.47 (m, 1H), 8.21-8.12 (m, 1H), 7.23-7.07 (m, 6H), 6.37-6.16 (m, 2H), 4.05 (dd, 1H), 3.19 (dd, 1H), 3.01 (s, 3H), 2.80-2.73 (m, 1H), 2.55 (s, 1H), 2.42-2.30 (m, 5H), 1.79-1.74 (m, 3H), 1.33 (s, 3H), 1.11 (s, 3H). LCMS (Method M): 501.1 [M+H]⁺.

### Example 180: (R)—N—((S)-1-(5-(((S)-1,1-Dimethyl-2,3-dihydro-1H-inden-2-yl)amino)pyridin-2-yl)-2,2,2-trifluoroethyl)-N,4-dimethylmorpholine-2-carboxamide

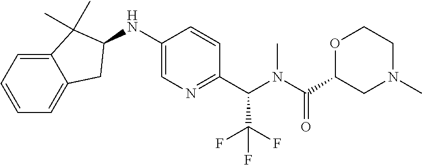 [SMILES: CN1CCO[C@@H](C(=O)N(C)[C@@H](c2ccc(N[C@H]3Cc4ccccc4C3(C)C)cn2)C(F)(F)F)C1]

A mixture of Intermediate 94 (70 mg, 0.13 mmol) and formaldehyde (37% aqueous, 12 μL, 0.16 mmol) in MeOH (1.6 mL) was stirred at RT for 15 min. Borane-pyridine complex (8M, 65 μL, 0.52 mmol) was added and the mixture was stirred at RT for 2 h. 1M aqueous HCl was added and stirring continued for 30 min. Aqueous K₂CO₃ was added until pH 7 the mixture was extracted with DCM. The organic layer washed with brine, dried over Na₂SO₄, filtered and concentrated under reduced pressure. The crude was purified by preparative HPLC (Gemini-NX C18, 21×250 mm×5 μm, flow rate: 30 mL/min, 0.1% aqueous formic acid with MeCN 30% to 80% over 12 min, held for 2 min, ramped to 95% over 0.2 min and held for 1.8 min) to provide the title compound (13 mg). ¹H NMR (400 MHz; DMSO-d₆) δ: 8.31 (s, 1H), 8.15 (d, 1H), 7.25-7.06 (m, 6H), 6.28 (dd, 2H), 4.36 (dd, 1H), 4.06 (m, 1H), 3.93-3.80 (m, 1H), 3.69-3.54 (m, 1H), 3.01 (s, 2H), 2.76-2.55 (m, 4H), 2.22-2.15 (m, 3H), 2.14 (m, 1H), 2.08-1.93 (m, 1H), 1.33 (s, 3H), 1.10 (s, 3H). LCMS (Method M): 477.1 [M+H]⁺.

### Example 181: (S)-4-Acetyl-N—((S)-1-(5-(((S)-1,1-dimethyl-2,3-dihydro-1H-inden-2-yl)amino)pyridin-2-yl)-2,2,2-trifluoroethyl)-N-methylmorpholine-2-carboxamide

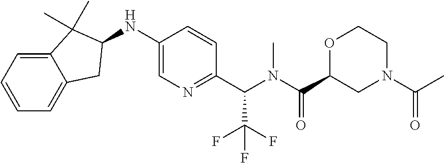 [SMILES: CC(=O)N1CCO[C@H](C(=O)N(C)[C@@H](c2ccc(N[C@H]3Cc4ccccc4C3(C)C)cn2)C(F)(F)F)C1]

The title compound (29 mg) was prepared in an analogous manner to Example 169 from Intermediate 96 (58 mg, 0.12 mmol), triethylamine (52 μL, 0.37 mmol) and acetyl chloride (1M in DCM, 0.15 mL, 0.15 mmol) in DCM (1.2 mL) at RT for 15 min. Purified by preparative HPLC (Gemini-NX C18, 21×250 mm×5 μm, flow rate: 30 mL/min, 0.1% aqueous formic acid with MeCN 50% to 60% over 6 min, held for 3 min, ramped to 95% over 0.2 min and held for 1.8 min). ¹H NMR (400 MHz; DMSO-d₆) δ: 8.16 (s, 1H), 7.30-7.10 (m, 6H), 6.30-6.20 (m, 2H), 4.43 (dd, 1H), 4.05 (m, 1H), 3.70-3.60 (m, 2H), 3.55-3.40 (m, 1H), 3.20 (dd, 1H), 3.03 (s, 3H), 2.85-2.55 (m, 4H), 2.05 (s, 3H), 1.33 (s, 3H), 1.11 (s, 3H). LCMS (Method M): 505.3 [M+H]⁺.

### Example 182: (1,3-cis)-3-Acetamido-N—((S)-1-(5-(((S)-1,1-dimethyl-2,3-dihydro-1H-inden-2-yl)amino)pyridin-2-yl)-2,2,2-trifluoroethyl)-N-methylcyclobutane-1-carboxamide

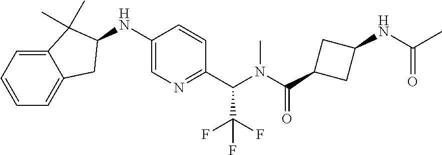 [SMILES: CC(=O)N[C@H]1C[C@@H](C(=O)N(C)[C@@H](c2ccc(N[C@H]3Cc4ccccc4C3(C)C)cn2)C(F)(F)F)C1]

The title compound (63 mg) was prepared in an analogous manner to Example 169 from Intermediate 98 (0.13 g, 0.29 mmol), triethylamine (81 μL, 0.58 mmol) and acetyl chloride (1M in DCM, 0.35 mL, 0.35 mmol) in DCM (1.5 mL) at RT for 16 h. Purified by preparative HPLC (Kinetex-XB C18, 21×250 mm×5 μm, flow rate: 30 mL/min, 0.1% aqueous formic acid with MeCN 40% to 63% over 6 min, held for 5 min, ramped to 95% over 0.2 min and held for 1.8 min). ¹H NMR (400 MHz; DMSO-d₆) δ: 8.15 (s, 1H), 8.14 (s, 1H), 7.29-7.02 (m, 6H), 6.35 (dd, 1H), 6.22 (d, 1H), 4.16 (dd, 1H), 4.05 (dd, 1H), 3.26-3.04 (m, 2H), 2.87 (s, 3H), 2.80-2.60 (m, 2H), 2.46-2.34 (m, 1H), 2.15-1.96 (m, 2H), 1.76 (s, 3H), 1.33 (s, 3H), 1.10 (s, 3H). LCMS (Method M): 489.2 [M+H]⁺.

### Example 183: (2S,4R)-1-Acetyl-N—((S)-1-(5-(((S)-1,1-dimethyl-2,3-dihydro-1H-inden-2-yl)amino)pyridin-2-yl)-2,2,2-trifluoroethyl)-4-hydroxy-N-methylpyrrolidine-2-carboxamide

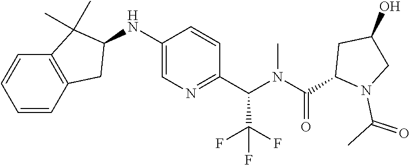 [SMILES: CC(=O)N1C[C@H](O)C[C@H]1C(=O)N(C)[C@@H](c1ccc(N[C@H]2Cc3ccccc3C2(C)C)cn1)C(F)(F)F]

The title compound (10 mg) was prepared in an analogous manner to Example 16 from Intermediate 100 (0.13 g, 0.29 mmol), TBAF (1M in THF, 0.48 mL, 0.48 mmol) in THF (0.8 mL) at RT for 30 min. Purified by preparative HPLC (Gemini NX-C18, 30×250 mm×5 μm, flow rate: 18 mL/min, 0.1% aqueous formic acid with 50% MeCN isocratic). ¹H NMR (400 MHz; DMSO-d₆) δ: 8.14 (d, 1H), 7.26-7.04 (m, 6H), 6.40-6.16 (m, 2H), 5.20-5.00 (m, 1H), 4.80-4.35 (m, 1H), 4.07 (dd, 1H), 3.66 (dd, 1H), 3.56-3.38 (m, 1H), 3.21 (dd, 1H), 3.02 (s, 3H), 2.81-2.73 (m, 1H), 2.18 (s, 1H), 1.95 (s, 3H), 1.91-1.70 (m, 2H), 1.33 (s, 3H), 1.11 (s, 3H). LCMS (Method M): 505.3 [M+H]⁺.

### Example 184: (1R,3S)—N—((S)-1-(5-(((S)-1,1-Dimethyl-2,3-dihydro-1H-inden-2-yl)amino)pyridin-2-yl)-2,2,2-trifluoroethyl)-N-methyl-3-(2,2,2-trifluoroacetamido)cyclopentane-1-carboxamide

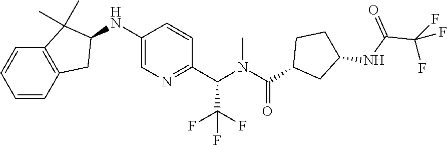 [SMILES: CN(C(=O)[C@@H]1CC[C@H](NC(=O)C(F)(F)F)C1)[C@@H](c1ccc(N[C@H]2Cc3ccccc3C2(C)C)cn1)C(F)(F)F]

To a stirred mixture of Intermediate 86 (0.13 g, 0.29 mmol) and triethylamine (0.15 mL, 1.10 mmol) in DCM (1.2 mL) was added acetyl chloride (1M in DCM, 0.55 mL, 0.55 mmol) and the mixture was stirred at RT for 1 h. The mixture was concentrated under reduced pressure and purified by prep-TLC (silica gel, eluting 30% EtOAc in hexane) to provide the title compound (12 mg). ¹H NMR (300 MHz; DMSO-d₆) δ: 9.43 (d, 1H), 8.15 (d, 1H), 7.30-7.04 (m, 6H), 6.39 (dd, 1H), 6.23 (d, 1H), 4.24-3.98 (m, 2H), 3.28-3.09 (m, 2H), 2.98 (s, 3H), 2.75 (dd, 2H), 2.04-1.62 (m, 5H), 1.33 (s, 3H), 1.10 (s, 3H). LCMS (Method M): 557.2 [M+H]⁺.

### Example 185: N¹—((S)-1-(5-(((S)-1,1-Dimethyl-2,3-dihydro-1H-inden-2-yl)amino)pyridin-2-yl)-2,2,2-trifluoroethyl)-N¹,N³-dimethylbicyclo[1.1.1]pentane-1,3-dicarboxamide

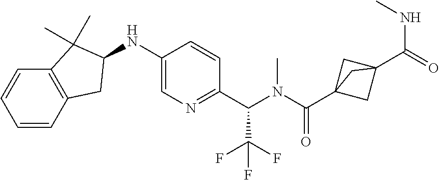 [SMILES: CNC(=O)C12CC(C(=O)N(C)[C@@H](c3ccc(N[C@H]4Cc5ccccc5C4(C)C)cn3)C(F)(F)F)(C1)C2]

A stirred mixture of Intermediate 101 (0.13 g, 0.29 mmol) and methylamine (2M in THF, 1.32 mL, 2.65 mmol) in MeOH (1 mL) was heated at 85° C. in a sealed tube for 16 h. The mixture was concentrated under reduced pressure and purified by by preparative HPLC (Kinetex-XB C18, 21×250 mm×5 μm, flow rate: 30 mL/min, 0.1% aqueous formic acid with MeCN 50% to 60% over 6 min, held for 5 min, ramped to 95% over 0.2 min and held for 1.8 min) to provide the title compound (35 mg). ¹H NMR (300 MHz; DMSO-d₆) δ: 8.16 (s, 1H), 7.80 (d, 1H), 7.31-7.02 (m, 6H), 6.40-6.20 (m, 2H), 4.05 (dd, 1H), 3.19 (dd, 1H), 3.03 (s, 3H), 2.84-2.64 (m, 1H), 2.57 (d, 3H), 2.32 (d, 1H), 2.40-2.20 (m, 5H), 1.33 (s, 3H), 1.11 (s, 3H). LCMS (Method M): 501.4 [M+H]⁺.

### Example 186: (1,3-trans)-3-Acetamido-N—((S)-1-(5-(((S)-1,1-dimethyl-2,3-dihydro-1H-inden-2-yl)amino)pyridin-2-yl)-2,2,2-trifluoroethyl)-N-methylcyclobutane-1-carboxamide

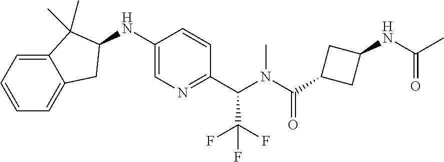 [SMILES: CC(=O)N[C@H]1C[C@H](C(=O)N(C)[C@@H](c2ccc(N[C@H]3Cc4ccccc4C3(C)C)cn2)C(F)(F)F)C1]

The title compound (29 mg) was prepared in an analogous manner to Example 169 from Intermediate 103 (77 mg, 0.17 mmol), triethylamine (72 μL, 0.52 mmol) and acetyl chloride (1M in DCM, 0.17 mL, 0.17 mmol) in DCM (1.1 mL) at RT for 15 min. Purified by preparative HPLC (Gemini NX-C18, 21×250 mm×5 μm, flow rate: 30 mL/min, 0.05% aqueous ammonia with MeCN 40% to 60% over 5.6 min, held for 4.5 min, ramped to 95% over 0.2 min and held for 2.7 min). ¹H NMR (300 MHz; DMSO-d₆) δ: 8.23-8.12 (m, 2H), 7.25-7.04 (m, 6H), 6.40 (dd, 1H), 6.22 (d, 1H), 4.19 (dd, 1H), 4.04 (dd, 1H), 3.20 (dd, 1H), 2.83 (s, 3H), 2.80-2.69 (m, 2H), 2.43-2.31 (m, 2H), 2.25-2.06 (m, 2H), 1.79 (s, 3H), 1.32 (s, 3H), 1.10 (s, 3H). LCMS (Method M): 489.2 [M+H]⁺.

### Example 187: (1,3-cis)-3-Acetamido-N—((S)-1-(5-(((S)-1,1-dimethyl-2,3-dihydro-1H-inden-2-yl)amino)pyridin-2-yl)-2,2,2-trifluoroethyl)-N-methylcyclobutane-1-carboxamide

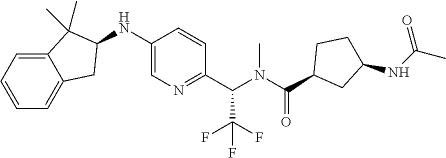 [SMILES: CC(=O)N[C@@H]1CC[C@H](C(=O)N(C)[C@@H](c2ccc(N[C@H]3Cc4ccccc4C3(C)C)cn2)C(F)(F)F)C1]

The title compound (18 mg) was prepared in an analogous manner to Example 169 from Intermediate 105 (58 mg, 0.13 mmol), triethylamine (53 μL, 0.38 mmol) and acetyl chloride (1M in DCM, 0.13 mL, 0.13 mmol) in DCM (1.7 mL) at RT for 15 min. Purified by preparative HPLC (Gemini NX-C18, 21×250 mm×5 μm, flow rate: 30 mL/min, 0.05% aqueous ammonia with MeCN 50% isocratic for 15 min, ramped to 95% over 0.2 min and held for 2.8 min). ¹H NMR (300 MHz; DMSO-d₆) δ: 8.15 (d, 1H), 7.90 (d, 1H), 7.23-7.06 (m, 6H), 6.40 (dd, 1H), 6.22 (d, 1H), 4.16-3.95 (m, 2H), 3.20 (dd, 2H), 2.96 (s, 3H), 2.82-2.68 (m, 2H), 2.25-2.09 (m, 1H), 1.88-1.79 (m, 2H), 1.78 (s, 3H), 1.59-1.37 (m, 2H), 1.32 (s, 3H), 1.10 (s, 3H). LCMS (Method M): 503.2 [M+H]⁺.

### Example 188: N-((1S)-1-(5-((1,1-Dimethyl-2,3-dihydro-1H-inden-2-yl)amino)pyridin-2-yl)-2,2,2-trifluoroethyl)-N-ethyltetrahydro-2H-thiopyran-4-carboxamide 1,1-dioxide—Isomer 1

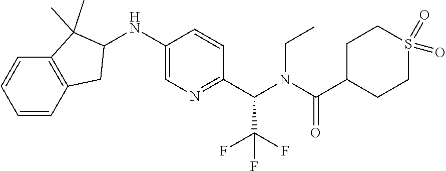 [SMILES: CCN(C(=O)C1CCS(=O)(=O)CC1)[C@@H](c1ccc(NC2Cc3ccccc3C2(C)C)cn1)C(F)(F)F]

### Example 189: N-((1S)-1-(5-((1,1-Dimethyl-2,3-dihydro-1H-inden-2-yl)amino)pyridin-2-yl)-2,2,2-trifluoroethyl)-N-ethyltetrahydro-2H-thiopyran-4-carboxamide 1,1-dioxide—Isomer 2

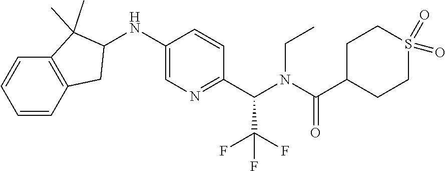 [SMILES: CCN(C(=O)C1CCS(=O)(=O)CC1)[C@@H](c1ccc(NC2Cc3ccccc3C2(C)C)cn1)C(F)(F)F]

The title compound was prepared in an analogous manner to Intermediate 11 from Intermediate 107 (0.62 g, 1.38 mmol), 1,1-dimethyl-2,3-dihydro-1H-inden-2-amine hydrochloride (0.44 g, 2.07 mmol, CAS 74413-86-8), Cs₂CO₃ (1.35 g, 4.14 mmol), and RuPhosPdG3 (0.12 g, 0.14 mmol) in toluene (12 mL) at 110° C. for 16 h. Purified by preparative HPLC (XBridge C18, 19×250 mm×5 μm, flow rate: 18 mL/min, 10 mM aqueous NH₄HCO₃ with MeCN 55% to 60% over 8 min, held for 4 min, ramped to 95% over 0.1 min and held for 1.9 min). Chiral purification was carried out by preparative SFC (Chiralpak® AD-H, 30×250 mm×5 μm, temp 30° C., Back Pressure 100 bar, flow rate: 100 g/min, 70% CO₂ with 30% MeOH modifier) to provide Example 188 (19 mg) as Peak 1 and Example 189 (22 mg) as Peak 2. Example 188: ¹H NMR (400 MHz; DMSO-d₆) δ: 8.18-8.16 (m, 1H), 7.26-7.13 (m, 6H), 6.39-5.96 (m, 2H), 4.09 (dd, 1H), 3.58-3.55 (m, 2H), 3.39-3.03 (m, 6H), 2.78-2.72 (m, 1H), 2.19-1.95 (m, 4H), 1.32 (s, 3H), 1.09 (s, 3H), 0.76-0.61 (m, 3H). LCMS (Method A): 524.4 [M+H]⁺. Example 189: ¹H NMR (400 MHz; DMSO-d₆) δ: 8.17-8.15 (m, 1H), 7.25-7.13 (m, 6H), 6.39-5.95 (m, 2H), 4.06 (dd, 1H), 3.57-3.55 (m, 2H), 3.39-3.06 (m, 6H), 2.77-2.71 (m, 1H), 2.13-1.95 (m, 4H), 1.31 (s, 3H), 1.10 (s, 3H), 0.76-0.61 (m, 3H). LCMS (Method A): 524.4 [M+H]⁺.

### Example 190: N-Methyl-N-((1S)-2,2,2-trifluoro-1-(5-((4-(trifluoromethyl)-2,3-dihydro-1H-inden-2-yl)amino)pyridin-2-yl)ethyl)tetrahydro-2H-thiopyran-4-carboxamide 1,1-dioxide—Isomer 1

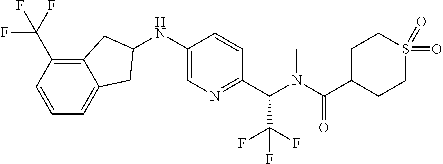 [SMILES: CN(C(=O)C1CCS(=O)(=O)CC1)[C@@H](c1ccc(NC2Cc3cccc(C(F)(F)F)c3C2)cn1)C(F)(F)F]

### Example 191: N-Methyl-N-((1S)-2,2,2-trifluoro-1-(5-((4-(trifluoromethyl)-2,3-dihydro-1H-inden-2-yl)amino)pyridin-2-yl)ethyl)tetrahydro-2H-thiopyran-4-carboxamide 1,1-dioxide—Isomer 2

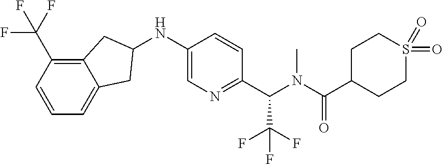 [SMILES: CN(C(=O)C1CCS(=O)(=O)CC1)[C@@H](c1ccc(NC2Cc3cccc(C(F)(F)F)c3C2)cn1)C(F)(F)F]

The title compound was prepared in an analogous manner to Intermediate 11 from Intermediate 90 (0.38 g, 0.88 mmol), Intermediate 109 (0.32 g, 1.31 mmol), Cs₂CO₃ (1.43 g, 4.38 mmol), and RuPhosPdG3 (0.15 g, 0.18 mmol) in toluene (5 mL) at 110° C. for 3 h. Purified by preparative HPLC (XBridge C18, 19×250 mm×5 μm, flow rate: 17 mL/min, 10 mM aqueous NH₄HCO₃ with MeCN 35% to 65% over 10 min, held for 6 min, ramped to 95% over 0.1 min and held for 4.9 min). Chiral purification was carried out by preparative SFC (Chiralpak® AD-H, 30×250 mm×5 μm, temp 30° C., Back Pressure 100 bar, flow rate: 100 g/min, 70% CO₂ with 30% MeOH modifier) to provide Example 190 (26 mg) as Peak 1 and Example 191 (24 mg) as Peak 2. Example 190: ¹H NMR (400 MHz; DMSO-d₆) δ: 8.07-8.02 (m, 1H), 7.57-7.51 (m, 2H), 7.42-7.38 (m, 1H), 7.28-7.14 (m, 1H), 7.10-7.03 (m, 1H), 6.57-6.52 (m, 1H), 6.42-6.04 (m, 1H), 4.38-4.33 (m, 1H), 3.52-3.38 (m, 2H), 3.32-3.12 (m, 5H), 3.09-2.86 (m, 4H), 2.68 (s, 1H), 2.17-1.95 (m, 4H). LCMS (Method A): 550.3 [M+H]⁺. Example 191: ¹H NMR (400 MHz; DMSO-d₆) δ: 8.06-8.02 (m, 1H), 7.57-7.51 (m, 2H), 7.42-7.38 (m, 1H), 7.28-7.14 (m, 1H), 7.10-7.03 (m, 1H), 6.57-6.52 (m, 1H), 6.42-6.04 (m, 1H), 4.38-4.33 (m, 1H), 3.52-3.38 (m, 2H), 3.32-3.12 (m, 5H), 3.09-2.86 (m, 4H), 2.68 (s, 1H), 2.17-1.95 (m, 4H). LCMS (Method A): 550.2 [M+H]⁺.

### Example 192: (R)-1-Acetyl-N—((S)-1-(5-(((S)-1,1-dimethyl-2,3-dihydro-1H-inden-2-yl)amino)pyridin-2-yl)-2,2,2-trifluoroethyl)-N-methylpyrrolidine-3-carboxamide

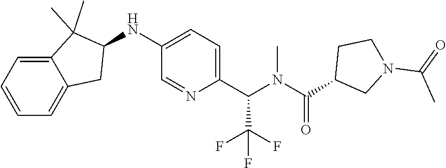 [SMILES: CC(=O)N1CC[C@@H](C(=O)N(C)[C@@H](c2ccc(N[C@H]3Cc4ccccc4C3(C)C)cn2)C(F)(F)F)C1]

The title compound (14 mg) was prepared in an analogous manner to Example 169 from Example 176 (50 mg, 0.11 mmol), triethylamine (47 μL, 0.34 mmol) and acetyl chloride (1M in DCM, 0.13 mL, 0.13 mmol) in DCM (0.7 mL) at RT for 15 min. Purified by preparative HPLC (Kinetex-XB C18, 21×250 mm×5 μm, flow rate: 30 mL/min, 0.1% aqueous formic acid with MeCN 50% isocratic for 11 min, ramped to 95% over 0.2 min, held for 1.8 min). ¹H NMR (300 MHz; DMSO-d₆) δ: 8.15 (d, 1H), 7.31-6.99 (m, 6H), 6.48-6.30 (m, 1H), 6.23 (d, 1H), 4.05 (dd, 1H), 3.68-3.45 (m, 3H), 3.19 (dd, 2H), 3.05-3.00 (m, 3H), 2.86-2.65 (m, 2H), 2.25-1.97 (m, 2H), 1.94 (s, 3H), 1.32 (s, 3H), 1.10 (s, 3H). LCMS (Method M): 489.2 [M+H]⁺.

### Example 193: (R)—N—((S)-1-(5-(((S)-1,1-Dimethyl-2,3-dihydro-1H-inden-2-yl)amino)pyridin-2-yl)-2,2,2-trifluoroethyl)-N-methylmorpholine-2-carboxamide

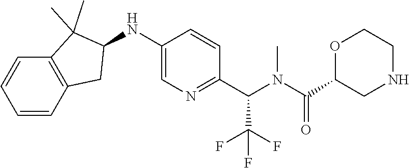 [SMILES: CN(C(=O)[C@H]1CNCCO1)[C@@H](c1ccc(N[C@H]2Cc3ccccc3C2(C)C)cn1)C(F)(F)F]

To a stirred solution of Intermediate 93 (0.15 g, 0.26 mmol) in 1,4-dioxane (2.6 mL) was added 4M HCl in 1,4-dioxane (0.39 mL) at RT for 16 h. The mixture concentrated under reduced pressure. The crude was purified by preparative HPLC (Gemini NX-C18, 21×250 mm×5 μm, flow rate: 30 mL/min, 0.05% aqueous ammonia with MeCN 50% isocratic for 15 min), to provide the title compound (10 mg). ¹H NMR (400 MHz; DMSO-d₆) δ: 8.33 (s, 1H), 8.15 (d, 1H), 7.26-7.05 (m, 6H), 6.35-5.95 (m, 2H), 4.37-4.17 (m, 1H), 4.12-3.99 (m, 1H), 3.76 (d 1H), 3.65-3.50 (m, 2H), 3.19 (dd, 1H), 3.00 (s, 2H), 2.93-2.63 (m, 5H), 1.33 (s, 3H), 1.10 (s, 3H). LCMS (Method M): 463.3 [M+H]⁺.

### Example 194: (S)-1-Acetyl-N—((S)-1-(5-(((S)-1,1-dimethyl-2,3-dihydro-1H-inden-2-yl)amino)pyridin-2-yl)-2,2,2-trifluoroethyl)-N-methylpyrrolidine-2-carboxamide

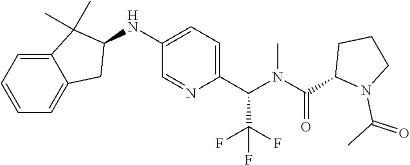 [SMILES: CC(=O)N1CCC[C@H]1C(=O)N(C)[C@@H](c1ccc(N[C@H]2Cc3ccccc3C2(C)C)cn1)C(F)(F)F]

The title compound (18 mg) was prepared in an analogous manner to Intermediate 3 from Intermediate 82 (50 mg, 0.14 mmol), pyridine (0.6 mL), T3P® (50% MeTHF, 0.27 mL, 0.43 mmol) and 1-acetylpyrrolidine-2-carboxylic acid (55 mg, 0.43 mmol, CAS 1074-79-9) at RT for 16 h. Purified by preparative HPLC (Gemini-NX-C18, 21×250 mm×5 μm, flow rate: 30 mL/min, 0.05% aqueous ammonia with MeCN 50% isocratic for 18 min) as the first Peak. ¹H NMR (400 MHz; DMSO-d₆) δ: 8.17 (d, 1H), 7.28-7.02 (m, 6H), 6.34 (br s, 1H), 5.91 (d, 1H), 4.95-4.60 (m, 1H), 4.05 (dd, 1H), 3.58 (dd, 1H), 3.42 (dd, 2H), 3.26 (dd, 1H), 2.78 (dd, 1H), 2.30-2.20 (m, 1H), 2.10-1.85 (m, 4H), 1.77 (s, 3H), 1.35 (s, 3H), 1.15-1.10 (m, 4H). LCMS (Method M): 489.2 [M+H]⁺.

### Example 195: (S)—N—((S)-1-(5-(((S)-1,1-Dimethyl-2,3-dihydro-1H-inden-2-yl)amino)pyridin-2-yl)-2,2,2-trifluoroethyl)-N,4-dimethylmorpholine-2-carboxamide

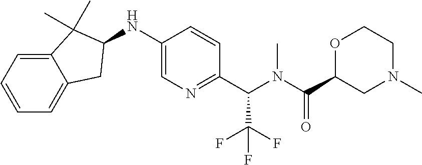 [SMILES: CN1CCO[C@H](C(=O)N(C)[C@@H](c2ccc(N[C@H]3Cc4ccccc4C3(C)C)cn2)C(F)(F)F)C1]

The title compound (39 mg) was prepared in an analogous manner to Example 180 from Intermediate 96 (76 mg, 0.14 mmol), formaldehyde (37% aqueous, 12 μL, 0.16 mmol) and borane-pyridine complex (8M, 36 μL, 0.29 mmol) in MeOH (1.4 mL) at RT for 2 h. Purified by preparative HPLC (Kinetex XBC18, 21×250 mm×5 μm, flow rate: 30 mL/min, 0.1% aqueous formic acid with MeCN 29% isocratic over 11 min, ramped to 95% over 0.2 min and held for 1.8 min). ¹H NMR (400 MHz; DMSO-d₆) δ: 8.30 (s, 1H), 8.15 (d, 1H), 7.24-7.13 (m, 5H), 7.08 (d, 1H), 6.36-6.10 (m, 2H), 4.48-4.30 (m, 1H), 4.05 (dd, 1H), 3.91-3.75 (m, 1H), 3.58 (dd, 1H), 3.20 (dd, 1H), 3.03 (s, 3H), 2.89-2.70 (m, 1H), 2.61 (dd, 1H), 2.21 (s, 3H), 2.12 (dd, 1H), 2.00 (dd, 1H), 1.33 (s, 3H), 1.11 (s, 3H). LCMS (Method M): 477.3 [M+H]⁺.

### Example 196: N—((S)-1-(5-(((S)-1,1-Dimethyl-2,3-dihydro-1H-inden-2-yl)amino)pyridin-2-yl)-2,2,2-trifluoroethyl)-2-(1,1-dioxidotetrahydro-2H-thiopyran-4-yl)-N-methylacetamide

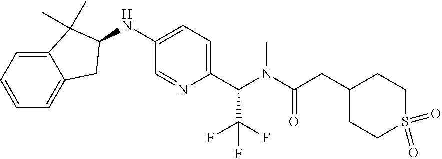 [SMILES: CN(C(=O)CC1CCS(=O)(=O)CC1)[C@@H](c1ccc(N[C@H]2Cc3ccccc3C2(C)C)cn1)C(F)(F)F]

The title compound (71 mg) was prepared in an analogous manner to Intermediate 3 from Intermediate 82 (50 mg, 0.14 mmol), pyridine (0.7 mL), T3P® (50% MeTHF, 0.26 mL, 0.43 mmol) and 2-(1,1-dioxidotetrahydro-2H-thiopyran-4-yl)acetic acid (30 mg, 0.16 mmol, CAS 1224869-02-6) at RT for 16 h. ¹H NMR (400 MHz; DMSO-d₆) δ: 8.17 (d, 1H), 7.25-7.05 (m, 6H), 6.50-6.40 (m, 1H), 5.92 (d, 1H), 4.04 (dd, 1H), 3.25 (dd, 1H), 3.23-2.80 (m, 6H), 2.77 (dd, 1H), 2.54 (s, 1H), 2.23-2.05 (m, 3H), 1.75 (dd, 2H), 1.35 (s, 3H), 1.28 (d, 2H), 1.15 (s, 3H). LCMS (Method M): 524.3 [M+H]⁺.

### Example 197: N-((1S)-1-(5-((4-Ethyl-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

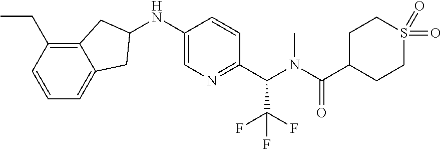 [SMILES: CCc1cccc2c1CC(Nc1ccc([C@H](N(C)C(=O)C3CCS(=O)(=O)CC3)C(F)(F)F)nc1)C2]

### Example 198: N-((1S)-1-(5-((4-Ethyl-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

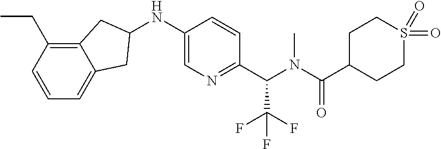 [SMILES: CCc1cccc2c1CC(Nc1ccc([C@H](N(C)C(=O)C3CCS(=O)(=O)CC3)C(F)(F)F)nc1)C2]

The title compound was prepared in an analogous manner to Intermediate 11 from Intermediate 90 (0.58 g, 1.06 mmol), Intermediate 111 (0.44 g, 2.12 mmol), Cs₂CO₃ (1.03 g, 3.17 mmol), and RuPhosPdG3 (89 mg, 0.11 mmol) in toluene (10 mL) at 110° C. for 16 h. Purified by preparative HPLC (XBridge C18, 19×250 mm×5 μm, flow rate: 18 mL/min, 10 mM aqueous NH₄HCO₃ with MeCN 40% to 60% over 10 min, held for 7 min, ramped to 98% over 0.1 min and held for 3.9 min). Chiral purification was carried out by preparative SFC (Chiralpak® AD-H, 30×250 mm×5 μm, temp 30° C., Back Pressure 100 bar, flow rate: 100 g/min, 65% CO₂ with 35% MeOH modifier) to provide Example 197 (38 mg) as Peak 1 and Example 198 (34 mg) as Peak 2. Example 197: ¹H NMR (400 MHz; DMSO-d₆) δ: 8.06-8.02 (m, 1H), 7.27-6.98 (m, 5H), 6.50-6.02 (m, 2H), 4.28-4.24 (m, 1H), 3.36-3.34 (m, 2H), 3.25-3.08 (m, 5H), 3.02 (s, 3H), 2.80 (dd, 1H), 2.74 (dd, 1H), 2.58-2.54 (m, 2H), 2.07-1.99 (m, 4H), 1.14 (t, 3H). LCMS (Method A): 510.4 [M+H]⁺. Example 198: ¹H NMR (400 MHz; DMSO-d₆) δ: 8.06-8.02 (m, 1H), 7.27-6.98 (m, 5H), 6.51-6.02 (m, 2H), 4.28-4.23 (m, 1H), 3.40-3.34 (m, 2H), 3.31-3.08 (m, 5H), 3.02 (s, 3H), 2.81 (dd, 1H), 2.74 (dd, 1H), 2.58-2.54 (m, 2H), 2.20-1.95 (m, 4H), 1.14 (t, 3H). LCMS (Method A): 510.3 [M+H]⁺.

### Example 199: N—((S)-1-(5-(((S)-5-Chloro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

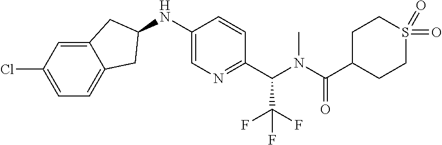 [SMILES: CN(C(=O)C1CCS(=O)(=O)CC1)[C@@H](c1ccc(N[C@H]2Cc3ccc(Cl)cc3C2)cn1)C(F)(F)F]

### Example 200: N—((S)-1-(5-(((R)-5-Chloro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

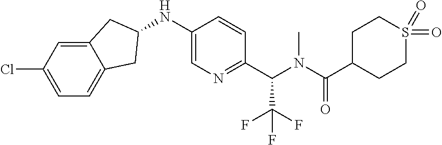 [SMILES: CN(C(=O)C1CCS(=O)(=O)CC1)[C@@H](c1ccc(N[C@@H]2Cc3ccc(Cl)cc3C2)cn1)C(F)(F)F]

The title compound was prepared in an analogous manner to Intermediate 3 from Intermediate 113 (0.57 g, 1.04 mmol), pyridine (1.0 mL), T3P® (50% MeTHF, 10 mL, 32.6 mmol) and tetrahydro-2H-thiopyran-4-carboxylic acid 1,1-dioxide (0.56 g, 3.12 mmol, CAS 64096-87-3) at RT for 16 h. Purified by preparative HPLC (XBridge C18, 19×250 mm×5 μm, flow rate: 18 mL/min, 10 mM aqueous NH₄HCO₃ with MeCN 40% to 58% over 9 min, held for 5 min, ramped to 100% over 0.1 min and held for 4.9 min). Chiral purification was carried out by preparative SFC (Chiralpak® AD-H, 30×250 mm×5 μm, temp 30° C., Back Pressure 100 bar, flow rate: 100 g/min, 65% CO₂ with 35% MeOH modifier) to provide Example 199 (0.16 g) as Peak 1 and Example 200 (0.15 g) as Peak 2. Example 199: ¹H NMR (400 MHz; DMSO-d₆) δ: 8.04-7.98 (m, 1H), 7.31 (s, 1H), 7.26 (d, 1H), 7.21 (d, 1H), 7.14 (d, 1H), 7.07-7.00 (m, 1H), 6.53-6.47 (m, 1H), 6.41-6.03 (m, 1H), 4.30-4.25 (m, 1H), 3.28-3.08 (m, 7H), 3.01 (s, 3H), 2.88-2.70 (m, 2H), 2.16-1.94 (m, 4H). LCMS (Method A): 516.4 [M+H]⁺. Example 200: ¹H NMR (400 MHz; DMSO-d₆) δ: 8.04-8.00 (m, 1H), 7.31 (s, 1H), 7.26 (d, 1H), 7.20 (d, 1H), 7.14 (d, 1H), 7.07-7.00 (m, 1H), 6.53-6.47 (m, 1H), 6.41-6.01 (m, 1H), 4.31-4.25 (m, 1H), 3.28-3.08 (m, 7H), 3.01 (s, 3H), 2.88-2.72 (m, 2H), 2.16-1.94 (m, 4H). LCMS (Method A): 516.3 [M+H]⁺.

### Example 201: N—((S)-1-(5-(((S)-1,1-Dimethyl-2,3-dihydro-1H-inden-2-yl)amino)pyridin-2-yl)-2,2,2-trifluoroethyl)-N,1-dimethyl-5-oxopiperazine-2-carboxamide—Isomer 1

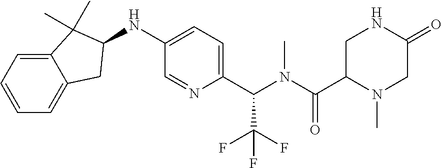 [SMILES: CN1CC(=O)NCC1C(=O)N(C)[C@@H](c1ccc(N[C@H]2Cc3ccccc3C2(C)C)cn1)C(F)(F)F]

### Example 202: N—((S)-1-(5-(((S)-1,1-Dimethyl-2,3-dihydro-1H-inden-2-yl)amino)pyridin-2-yl)-2,2,2-trifluoroethyl)-N,1-dimethyl-5-oxopiperazine-2-carboxamide—Isomer 2

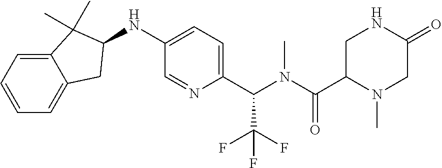 [SMILES: CN1CC(=O)NCC1C(=O)N(C)[C@@H](c1ccc(N[C@H]2Cc3ccccc3C2(C)C)cn1)C(F)(F)F]

The title compound was prepared in an analogous manner to Intermediate 3 from Intermediate 82 (0.10 g, 0.29 mmol), pyridine (1.2 mL), T3P® (50% EtOAc, 0.18 mL, 0.29 mmol) and 1-methyl-5-oxo-piperazine-2-carboxylic acid (50 mg, 0.31 mmol, CAS 1698289-73-4) at RT for 16 h. Purified by preparative HPLC (Kinetex XB-C18, 21×250 mm×5 μm, flow rate: 30 mL/min, 0.1% aqueous formic acid with MeCN 40% isocratic for 10 min, ramped to 95% over 0.2 min and held for 1.8 min), to provide Example 201 (0.16 g) as Peak 1 and Example 202 (0.15 g) as Peak 2. Example 201: ¹H NMR (400 MHz; 353 K, DMSO-d₆) δ: 8.54 (t, 1H), 7.48-7.30 (m, 1H), 7.28-6.95 (m, 6H), 6.35 (s, 1H), 5.93 (d, 1H), 4.09-3.87 (m, 2H), 3.55-3.45 (m, 1H), 3.35 (ddd, 1H), 3.30-3.18 (m, 3H), 2.77 (dd, 1H), 2.54 (s, 3H), 2.30 (s, 3H), 1.34 (s, 3H), 1.14 (s, 3H). LCMS (Method M): 490.2 [M+H]⁺. Example 202: ¹H NMR (400 MHz; 353 K, DMSO-d₆) δ: 8.50 (br s, 1H), 7.41 (br s, 1H), 7.23-7.09 (m, 6H), 6.40-6.30 (m, 1H), 5.93 (d, 1H), 4.12-3.92 (m, 2H), 3.58-3.47 (m, 1H), 3.31 (ddd, 1H), 3.29-3.17 (m, 3H), 2.77 (dd, 1H), 2.53 (s, 3H), 2.33 (s, 3H), 1.34 (s, 3H), 1.14 (s, 3H). LCMS (Method M): 490.2 [M+H]⁺.

### Example 203: N¹—((S)-1-(5-(((R)-1,1-Dimethyl-2,3-dihydro-1H-inden-2-yl)amino)pyridin-2-yl)-2,2,2-trifluoroethyl)-N¹,N³-dimethylbicyclo[1.1.1]pentane-1,3-dicarboxamide

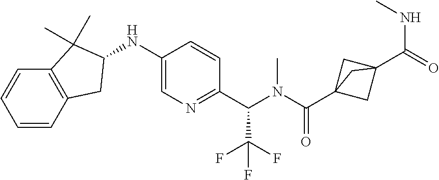 [SMILES: CNC(=O)C12CC(C(=O)N(C)[C@@H](c3ccc(N[C@@H]4Cc5ccccc5C4(C)C)cn3)C(F)(F)F)(C1)C2]

A stirred mixture of Intermediate 117 (0.17 g, 0.33 mmol) and methylamine (2M in THF, 1.65 mL, 3.31 mmol) was heated at 85° C. in a sealed tube for 16 h. The mixture was concentrated under reduced pressure and purified by by preparative HPLC (Gemini NX-C18, 21×250 mm×5 μm, flow rate: 30 mL/min, water with MeCN 40% to 58% over 6 min, held for 5 min) to provide the title compound (32 mg). ¹H NMR (300 MHz; DMSO-d₆) δ: 8.16 (s, 1H), 7.80 (s, 1H), 7.27-7.06 (m, 6H), 6.38-6.18 (m, 2H), 4.04 (dd, 1H), 3.20 (dd, 1H), 3.03 (s, 3H), 2.77-2.65 (m, 1H), 2.60-2.55 (m, 3H), 2.38-2.18 (m, 6H), 1.32 (s, 3H), 1.10 (s, 3H). LCMS (Method M): 501.2 [M+H]⁺.

### Example 204: (1R,3S)-3-Acetamido-N—((S)-1-(5-(((R)-1,1-dimethyl-2,3-dihydro-1H-inden-2-yl)amino)pyridin-2-yl)-2,2,2-trifluoroethyl)-N-methylcyclopentane-1-carboxamide

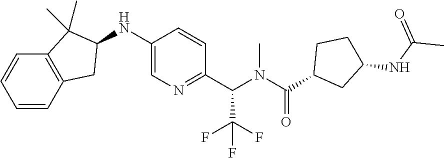 [SMILES: CC(=O)N[C@H]1CC[C@@H](C(=O)N(C)[C@@H](c2ccc(N[C@H]3Cc4ccccc4C3(C)C)cn2)C(F)(F)F)C1]

The title compound (27 mg) was prepared in an analogous manner to Example 169 from Intermediate 119 (0.10 g, 0.13 mmol), triethylamine (55 μL, 0.39 mmol) and acetyl chloride (1M in DCM, 0.16 mL, 0.16 mmol) in DCM (1.3 mL) at RT for 15 min. Purified by preparative HPLC (Gemini NX-C18, 21×250 mm×5 μm, flow rate: 30 mL/min, 0.1% aqueous formic acid with MeCN 48% isocratic for 13 min, ramped to 95% over 0.2 min and held for 2.8 min). ¹H NMR (400 MHz; DMSO-d₆) δ: 8.15 (d, 1H), 7.91 (d, 1H), 7.24-7.02 (m, 6H), 6.41 (dd, 1H), 6.22 (d, 1H), 4.04 (dd, 2H), 3.25-3.12 (m, 2H), 2.97 (s, 3H), 2.83-2.70 (m, 1H), 2.10-2.00 (m, 1H), 1.96-1.83 (m, 2H), 1.81-1.70 (m, 4H), 1.70-1.50 (m, 1H), 1.50-1.40 (m, 1H), 1.33 (s, 3H), 1.11 (s, 3H). LCMS (Method M): 503.2 [M+H]⁺.

### Example 205: (1,3-cis)-3-Acetamido-N—((S)-1-(5-(((R)-1,1-dimethyl-2,3-dihydro-1H-inden-2-yl)amino)pyridin-2-yl)-2,2,2-trifluoroethyl)-N-methylcyclobutane-1-carboxamide

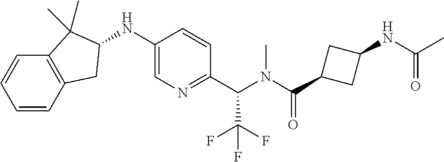 [SMILES: CC(=O)N[C@H]1C[C@@H](C(=O)N(C)[C@@H](c2ccc(N[C@@H]3Cc4ccccc4C3(C)C)cn2)C(F)(F)F)C1]

The title compound (51 mg) was prepared in an analogous manner to Example 169 from Intermediate 121 (90 mg, 0.17 mmol), triethylamine (97 μL, 0.69 mmol) and acetyl chloride (1M in DCM, 0.18 mL, 0.18 mmol) in DCM (0.9 mL) at RT for 1 h. Purified by preparative HPLC (Gemini NX-C18, 21×250 mm×5 μm, flow rate: 30 mL/min, 0.1% aqueous formic acid with MeCN 40% isocratic). ¹H NMR (300 MHz; DMSO-d₆) δ: 8.27-8.07 (m, 2H), 7.27-7.06 (m, 6H), 6.40 (dd, 1H), 6.21 (d, 1H), 4.19 (dd, 1H), 4.03 (dd, 1H), 3.20 (dd, 1H), 2.83 (s, 3H), 2.75 (dd, 2H), 2.43-2.33 (m, 2H), 2.26-2.05 (m, 2H), 1.79 (s, 3H), 1.32 (s, 3H), 1.11 (s, 3H). LCMS (Method M): 489.2 [M+H]⁺.

### Example 206: (1,3-trans)-3-Acetamido-N—((S)-1-(5-(((R)-1,1-dimethyl-2,3-dihydro-1H-inden-2-yl)amino)pyridin-2-yl)-2,2,2-trifluoroethyl)-N-methylcyclobutane-1-carboxamide

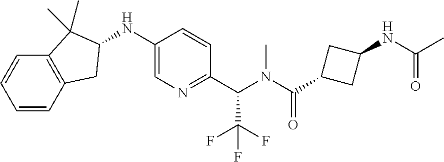 [SMILES: CC(=O)N[C@H]1C[C@H](C(=O)N(C)[C@@H](c2ccc(N[C@@H]3Cc4ccccc4C3(C)C)cn2)C(F)(F)F)C1]

The title compound (51 mg) was prepared in an analogous manner to Example 169 from Intermediate 123 (90 mg, 0.17 mmol), triethylamine (97 μL, 0.69 mmol) and acetyl chloride (1M in DCM, 0.18 mL, 0.18 mmol) in DCM (0.9 mL) at RT for 1 h. ¹H NMR (300 MHz; DMSO-d₆) δ: 8.15 (s, 2H), 7.34-7.02 (m, 6H), 6.35 (dd, 1H), 6.21 (d, 1H), 4.26-4.10 (m, 1H), 4.04 (dd, 1H), 3.28-3.03 (m, 2H), 2.87 (s, 3H), 2.74 (dd, 2H), 2.43-2.32 (m, 1H), 2.14-2.06 (m, 2H), 1.76 (s, 3H), 1.32 (s, 3H), 1.10 (s, 3H). LCMS (Method M): 489.3 [M+H]⁺.

### Example 207: (2,4-cis)-N-((1S)-1-(5-((1,1-Dimethyl-2,3-dihydro-1H-inden-2-yl)amino)pyridin-2-yl)-2,2,2-trifluoroethyl)-N-methyl-6-oxo-7-oxa-5-azaspiro[3.4]octane-2-carboxamide—Isomer 1

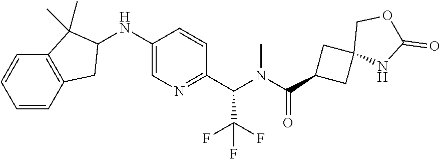 [SMILES: CN(C(=O)[C@H]1C[C@@]2(COC(=O)N2)C1)[C@@H](c1ccc(NC2Cc3ccccc3C2(C)C)cn1)C(F)(F)F]

### Example 208: (2,4-cis)-N-((1S)-1-(5-((1,1-Dimethyl-2,3-dihydro-1H-inden-2-yl)amino)pyridin-2-yl)-2,2,2-trifluoroethyl)-N-methyl-6-oxo-7-oxa-5-azaspiro[3.4]octane-2-carboxamide—Isomer 2

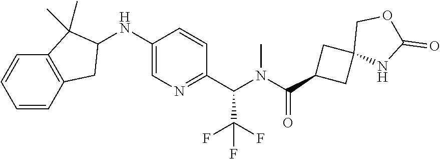 [SMILES: CN(C(=O)[C@H]1C[C@@]2(COC(=O)N2)C1)[C@@H](c1ccc(NC2Cc3ccccc3C2(C)C)cn1)C(F)(F)F]

The title compound was prepared in an analogous manner to Intermediate 3 from Intermediate 61 (0.45 g, 1.17 mmol), pyridine (0.28 mL, 3.51 mmol), T3P® (1.72 mL, 5.84 mmol) and cis-6-oxo-7-oxa-5-azaspiro[3.4]octane-2-carboxylic acid (0.20 g, 1.17 mmol, CAS 2135785-62-3) at RT for 16 h. Purified by preparative HPLC (XBridge C18, 19×250 mm×5 μm, flow rate: 18 mL/min, 10 mM aqueous NH₄HCO₃ with MeCN 40% to 65% over 10 min, held for 2.5 min, ramped to 100% over 0.2 min and held for 3.3 min). Chiral purification was carried out by preparative SFC (LUX Cellulose-C4, 30×250 mm×5 μm, temp 30° C., Back Pressure 100 bar, flow rate: 100 g/min, 55% CO₂ with 45% MeOH modifier) to provide Example 207 (69 mg) as Peak 1 and Example 208 (59 mg) as Peak 2. Example 207: ¹H NMR (400 MHz; DMSO-d₆) δ: 8.14 (d, 1H), 8.06 (s, 1H), 7.21-7.07 (m, 6H), 6.36-6.19 (m, 2H), 4.40-4.33 (m, 2H), 4.03 (dd, 1H), 3.20-3.12 (m, 2H), 2.88 (s, 3H), 2.77-2.66 (m, 1H), 2.44-2.35 (m, 4H), 1.31 (s, 3H), 1.09 (s, 3H). LCMS (Method Q): 503.4 [M+H]⁺. Example 208: ¹H NMR (400 MHz; DMSO-d₆) δ: 8.13 (d, 1H), 8.06 (s, 1H), 7.20-7.07 (m, 6H), 6.36-6.20 (m, 2H), 4.41-4.34 (m, 2H), 4.04 (dd, 1H), 3.31-3.12 (m, 2H), 2.87 (s, 3H), 2.78-2.70 (m, 1H), 2.44-2.35 (m, 4H), 1.31 (s, 3H), 1.09 (s, 3H). LCMS (Method Q): 503.4 [M+H]⁺.

### Example 209: N-((1S)-1-(5-((1,1-Dimethyl-2,3-dihydro-1H-inden-2-yl)amino)pyridin-2-yl)-2,2,2-trifluoroethyl)-N-methyl-1-(methylsulfonyl)azetidine-3-carboxamide—Isomer 1

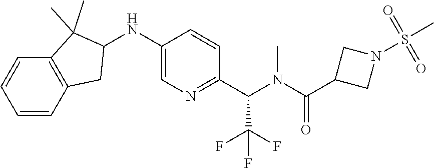 [SMILES: CN(C(=O)C1CN(S(C)(=O)=O)C1)[C@@H](c1ccc(NC2Cc3ccccc3C2(C)C)cn1)C(F)(F)F]

### Example 210: N-((1S)-1-(5-((1,1-Dimethyl-2,3-dihydro-1H-inden-2-yl)amino)pyridin-2-yl)-2,2,2-trifluoroethyl)-N-methyl-1-(methylsulfonyl)azetidine-3-carboxamide—Isomer 2

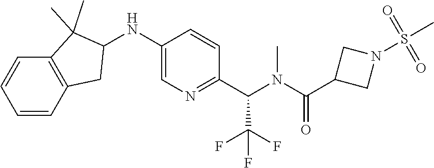 [SMILES: CN(C(=O)C1CN(S(C)(=O)=O)C1)[C@@H](c1ccc(NC2Cc3ccccc3C2(C)C)cn1)C(F)(F)F]

The title compound was prepared in an analogous manner to Intermediate 3 from Intermediate 61 (0.20 g, 0.54 mmol), pyridine (0.2 mL), T3P® (50% in EtOAc, 4.0 mL, 13.4 mmol) and 1-(methylsulfonyl)azetidine-3-carboxylic acid (0.15 g, 0.82 mmol, CAS 1219828-27-9) at RT for 16 h. Purified by preparative HPLC (XBridge C18, 19×150 mm×5 μm, flow rate: 18 mL/min, 10 mM aqueous NH₄HCO₃ with MeCN 45% to 60% over 6 min, held for 8 min, ramped to 100% over 0.1 min and held for 1.9 min). Chiral purification was carried out by preparative SFC (Chiralpak®-AD-H, 30×250 mm×5 μm, temp 30° C., Back Pressure 100 bar, flow rate: 100 g/min, 65% CO₂ with 35% MeOH modifier) to provide Example 209 (54 mg) as Peak 1 and Example 210 (62 mg) as Peak 2. Example 209: ¹H NMR (400 MHz; DMSO-d₆) δ: 8.15 (s, 1H), 7.21-7.13 (m, 6H), 6.24-5.65 (m, 2H), 4.07-3.87 (m, 6H), 3.22-3.15 (m, 1H), 3.00 (s, 3H), 2.83 (s, 3H), 2.78-2.73 (m, 1H), 1.32 (s, 3H), 1.10 (s, 3H). LCMS (Method Q): 511.4 [M+H]⁺. Example 210: ¹H NMR (400 MHz; DMSO-d₆) δ: 8.15 (s, 1H), 7.21-7.13 (m, 6H), 6.37-5.65 (m, 2H), 4.04-3.86 (m, 6H), 3.22-3.16 (m, 1H), 3.00 (s, 3H), 2.84 (s, 3H), 2.77-2.66 (m, 1H), 1.32 (s, 3H), 1.10 (s, 3H). LCMS (Method Q): 511.4 [M+H]⁺.

### Example 211: (S)—N—((S)-1-(5-(((R)-1,1-Dimethyl-2,3-dihydro-1H-inden-2-yl)amino)pyridin-2-yl)-2,2,2-trifluoroethyl)-N-methyl-5-oxopyrrolidine-3-carboxamide

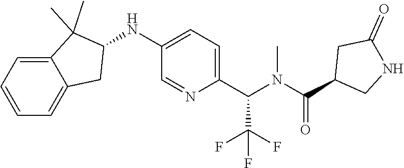 [SMILES: CN(C(=O)[C@@H]1CNC(=O)C1)[C@@H](c1ccc(N[C@@H]2Cc3ccccc3C2(C)C)cn1)C(F)(F)F]

The title compound (41 mg) was prepared in an analogous manner to Intermediate 3 from Intermediate 116 (0.10 g, 0.28 mmol), pyridine (1.1 mL), T3P® (50% MeTHF, 0.55 mL, 0.86 mmol) and (3S)-5-oxopyrrolidine-3-carboxylic acid (40 mg, 0.31 mmol, CAS 30948-17-5) at RT for 65 h. Purified by preparative HPLC (Gemini NX-C18, 21×250 mm×5 μm, flow rate: 30 mL/min, 0.1% aqueous formic acid with MeCN 30% to 65% over 6 min, held for 3 min, ramped to 95% over 0.2 min and held for 1.8 min). ¹H NMR (400 MHz; DMSO-d₆) δ: 7.70-7.55 (m, 1H), 7.24-7.07 (m, 6H), 6.37 (dd, 1H), 6.24 (d, 1H), 4.70-3.70 (m, 1H), 3.82-3.65 (m, 1H), 3.57 (dd, 1H), 3.31-3.10 (m, 3H), 2.97 (s, 3H), 2.75 (dd, 1H), 2.45-2.28 (m, 2H), 1.32 (s, 3H), 1.11 (s, 3H). LCMS (Method M): 461.4 [M+H]⁺.

### Example 212: N-((1S)-1-(5-((1,1-Dimethyl-2,3-dihydro-1H-inden-2-yl)amino)pyridin-2-yl)-2,2,2-trifluoroethyl)-2-(2,4-dioxoimidazolidin-1-yl)-N-methylacetamide—Isomer 1

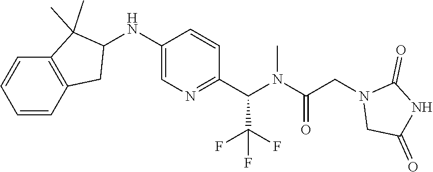 [SMILES: CN(C(=O)CN1CC(=O)NC1=O)[C@@H](c1ccc(NC2Cc3ccccc3C2(C)C)cn1)C(F)(F)F]

### Example 213: N-((1S)-1-(5-((1,1-Dimethyl-2,3-dihydro-1H-inden-2-yl)amino)pyridin-2-yl)-2,2,2-trifluoroethyl)-2-(2,4-dioxoimidazolidin-1-yl)-N-methylacetamide—Isomer 2

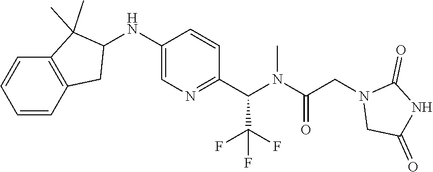 [SMILES: CN(C(=O)CN1CC(=O)NC1=O)[C@@H](c1ccc(NC2Cc3ccccc3C2(C)C)cn1)C(F)(F)F]

The title compound was prepared in an analogous manner to Intermediate 3 from Intermediate 61 (0.44 g, 1.14 mmol), pyridine (0.2 mL), T3P® (1.41 mL, 4.74 mmol) and 2-(2,4-dioxoimidazolidin-1-yl)acetic acid (0.15 g, 0.95 mmol, CAS 94738-31-5) at RT for 16 h. Chiral purification was carried out by preparative SFC (Chiralcel-OJ-H, 30×250 mm×5 μm, temp 30° C., Back Pressure 100 bar, flow rate: 100 g/min, 70% CO₂ with 30% MeOH modifier) to provide Example 212 (61 mg) as Peak 1 and Example 213 (64 mg) as Peak 2. Example 212: ¹H NMR (400 MHz; DMSO-d₆) δ: 10.93 (s, 1H), 8.15 (s, 1H), 7.20-7.12 (m, 6H), 6.32-5.85 (m, 2H), 4.42-4.30 (m, 2H), 3.97-3.87 (m, 3H), 3.18 (dd, 1H), 2.98 (s, 3H), 2.78-2.71 (m, 1H), 1.31 (s, 3H), 1.09 (s, 3H). LCMS (Method Q): 490.4 [M+H]⁺. Example 213: ¹H NMR (400 MHz; DMSO-d₆) δ: 10.93 (s, 1H), 8.15 (s, 1H), 7.22-7.14 (m, 6H), 6.28-5.83 (m, 2H), 4.42-4.30 (m, 2H), 4.08-3.87 (m, 3H), 3.18 (dd, 1H), 2.98 (s, 3H), 2.78-2.71 (m, 1H), 1.32 (s, 3H), 1.09 (s, 3H). LCMS (Method Q): 490.4 [M+H]⁺.

### Example 214: N-((1S)-1-(5-((1,1-Dimethyl-2,3-dihydro-1H-inden-2-yl)amino)pyridin-2-yl)-2,2,2-trifluoroethyl)-N-methyl-3-thiabicyclo[3.1.0]hexane-6-carboxamide 3,3-dioxide—Isomer 1

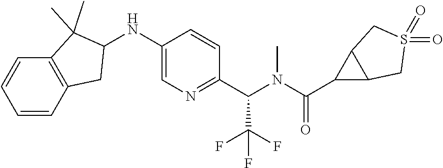 [SMILES: CN(C(=O)C1C2CS(=O)(=O)CC21)[C@@H](c1ccc(NC2Cc3ccccc3C2(C)C)cn1)C(F)(F)F]

### Example 215: N-((1S)-1-(5-((1,1-Dimethyl-2,3-dihydro-1H-inden-2-yl)amino)pyridin-2-yl)-2,2,2-trifluoroethyl)-N-methyl-3-thiabicyclo[3.1.0]hexane-6-carboxamide 3,3-dioxide—Isomer 2

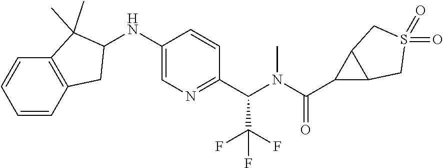 [SMILES: CN(C(=O)C1C2CS(=O)(=O)CC21)[C@@H](c1ccc(NC2Cc3ccccc3C2(C)C)cn1)C(F)(F)F]

### Example 216: N-((1S)-1-(5-((1,1-Dimethyl-2,3-dihydro-1H-inden-2-yl)amino)pyridin-2-yl)-2,2,2-trifluoroethyl)-N-methyl-3-thiabicyclo[3.1.0]hexane-6-carboxamide 3,3-dioxide—Isomer 3

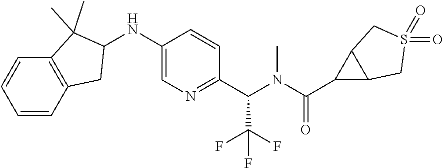 [SMILES: CN(C(=O)C1C2CS(=O)(=O)CC21)[C@@H](c1ccc(NC2Cc3ccccc3C2(C)C)cn1)C(F)(F)F]

### Example 217: N-((1S)-1-(5-((1,1-Dimethyl-2,3-dihydro-1H-inden-2-yl)amino)pyridin-2-yl)-2,2,2-trifluoroethyl)-N-methyl-3-thiabicyclo[3.1.0]hexane-6-carboxamide 3,3-dioxide—Isomer 4

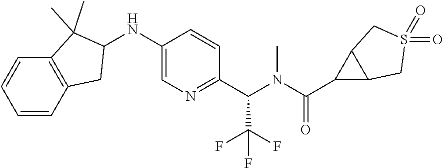 [SMILES: CN(C(=O)C1C2CS(=O)(=O)CC21)[C@@H](c1ccc(NC2Cc3ccccc3C2(C)C)cn1)C(F)(F)F]

The title compound was prepared in an analogous manner to Intermediate 3 from Intermediate 61 (0.30 g, 0.82 mmol), pyridine (0.3 mL), T3P® (50% in EtOAc, 5.7 mL, 17.9 mmol) and 3-thiabicyclo[3.1.0]hexane-6-carboxylic acid 3,3-dioxide (0.22 g, 1.22 mmol, CAS 1783317-08-7) at RT for 16 h. Purified by preparative HPLC (Gemini NX-C18, 19×250 mm×5 μm, flow rate: 18 mL/min, 10 mM aqueous NH₄HCO₃ with MeCN 50% to 85% over 9 min, held for 1.5 min, ramped to 100% over 0.1 min and held for 2.4 min). Chiral purification was carried out by preparative SFC (Lux Cellulose-4, 30×250 mm×5 μm, temp 30° C., Back Pressure 100 bar, flow rate: 100 g/min, 75% CO₂ with 25% MeOH modifier) to provide Example 214 (42 mg) as Peak 1, Example 215 (33 mg) as Peak 2, Example 216 (85 mg) as Peak 3, Example 217 (88 mg) as Peak 4. Example 214: ¹H NMR (400 MHz; DMSO-d₆) δ: 8.17-8.15 (m, 1H), 7.20-7.12 (m, 6H), 6.40-6.13 (m, 2H), 4.05 (dd, 1H), 3.48-3.38 (m, 2H), 3.23-3.13 (m, 2H), 3.04-2.67 (m, 5H), 2.54-2.51 (m, 1H), 2.33-2.24 (m, 2H), 1.32 (s, 3H), 1.10 (s, 3H). LCMS (Method Q): 508.4 [M+H]⁺. Example 215: ¹H NMR (400 MHz; DMSO-d₆) δ: 8.17-8.15 (m, 1H), 7.20-7.12 (m, 6H), 6.40-6.13 (m, 2H), 4.07 (dd, 1H), 3.48-3.38 (m, 2H), 3.22-3.12 (m, 2H), 3.06-2.67 (m, 5H), 2.54-2.40 (m, 1H), 2.51-2.24 (m, 2H), 1.32 (s, 3H), 1.10 (s, 3H). LCMS (Method Q): 508.4 [M+H]⁺. Example 216: ¹H NMR (400 MHz; DMSO-d₆) δ: 8.18-8.14 (m, 1H), 7.22-7.09 (m, 6H), 6.33-6.22 (m, 2H), 4.06 (dd, 1H), 3.59-3.51 (m, 2H), 3.22-2.67 (m, 7H), 2.42 (dd, 1H), 2.16-2.13 (m, 2H), 1.32 (s, 3H), 1.01 (s, 3H). LCMS (Method Q): 508.4 [M+H]⁺. Example 217: ¹H NMR (400 MHz; DMSO-d₆) δ: 8.18-8.15 (m, 1H), 7.21-7.08 (m, 6H), 6.33-6.21 (m, 2H), 4.05 (dd, 1H), 3.59-3.51 (m, 2H), 3.22-3.16 (m, 1H), 3.12 (s, 3H), 3.07-3.00 (m, 1H), 2.77-2.70 (m, 2H), 2.43 (dd, 1H), 2.17-2.13 (m, 2H), 1.32 (s, 3H), 1.01 (s, 3H). LCMS (Method Q): 508.4 [M+H]⁺.

### Example 218: (R)—N—((S)-1-(6-(((S)-1,1-dimethyl-2,3-dihydro-1H-inden-2-yl)amino)pyridin-3-yl)-2,2,2-trifluoroethyl)-N-methyl-6-oxopiperidine-3-carboxamide

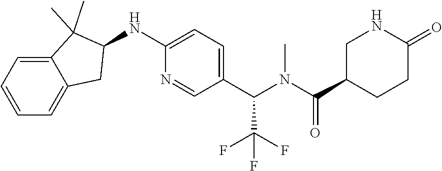 [SMILES: CN(C(=O)[C@@H]1CCC(=O)NC1)[C@@H](c1ccc(N[C@H]2Cc3ccccc3C2(C)C)nc1)C(F)(F)F]

The title compound (5 mg) was prepared in an analogous manner to Intermediate 3 from Intermediate 124 (50 mg, 0.14 mmol), pyridine (0.7 mL), T3P® (50% MeTHF, 0.13 mL, 0.43 mmol) and (R)-6-oxopiperidine-3-carboxylic acid (23 mg, 0.16 mmol, CAS 1426408-55-0) at RT for 24 h. Purified by preparative HPLC (Gemini NX-C18, 21×250 mm×5 μm, flow rate: 29 mL/min, 0.05% aqueous ammonia with MeCN 45% to 50% over 6 min, held for 10 min, ramped to 95% over 0.2 min and held for 1.8 min). ¹H NMR (400 MHz; 353 K, DMSO-d₆) δ: 7.99 (s, 1H), 7.43-7.36 (m, 1H), 7.24-7.10 (m, 5H), 6.70 (d, 1H), 6.60 (d, 1H), 6.40-6.25 (m, 1H), 4.59 (dd, 1H), 3.40-3.14 (m, 4H), 2.94 (s, 3H), 2.79 (dd, 1H), 2.31-2.19 (m, 2H), 2.03-1.91 (m, 1H), 1.88-1.77 (m, 1H), 1.32 (s, 3H), 1.12 (s, 3H). LCMS (Method M): 475.3 [M+H]⁺

### Example 219: (S)—N—((S)-1-(6-(((S)-1,1-dimethyl-2,3-dihydro-1H-inden-2-yl)aminopyridin-3-yl)-2,2,2-trifluoroethyl)-N-methyl-6-oxopiperidine-3-carboxamide

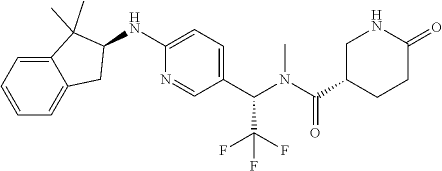 [SMILES: CN(C(=O)[C@H]1CCC(=O)NC1)[C@@H](c1ccc(N[C@H]2Cc3ccccc3C2(C)C)nc1)C(F)(F)F]

The title compound (7 mg) was prepared in an analogous manner to Intermediate 3 from Intermediate 124 (70 mg, 0.20 mmol), pyridine (1.0 mL), T3P® (50% MeTHF, 0.18 mL, 0.60 mmol) and (S)-6-oxopiperidine-3-carboxylic acid (23 mg, 0.16 mmol, CAS 1426408-56-1) at RT for 24 h. Purified by preparative HPLC (Gemini NX-C18, 21×250 mm×5 μm, flow rate: 30 mL/min, 0.05% aqueous ammonia with MeCN 45% to 50% over 6 min, held for 5 min, ramped to 95% over 0.2 min and held for 1.8 min). ¹H NMR (300 MHz; DMSO-d₆) δ: 7.94 (s, 1H), 7.50 (s, 1H), 7.38 (d, 1H), 7.25-7.10 (m, 4H), 6.93 (d, 1H), 6.68 (d, 1H), 6.36 (dd, 1H), 4.59 (dd, 1H), 3.40-3.10 (m, 5H), 2.96 (s, 3H), 2.80-2.20 (m, 2H), 2.25-2.10 (m, 1H), 1.85-1.72 (m, 1H), 1.30 (s, 3H), 1.09 (s, 3H). LCMS (Method M): 475.3 [M+H]⁺.

### Example 220: (2S,4R)-1-Acetyl-N—((S)-1-(5-(((R)-1,1-dimethyl-2,3-dihydro-1H-inden-2-yl)amino)pyridin-2-yl)-2,2,2-trifluoroethyl)-4-hydroxy-N-methylpyrrolidine-2-carboxamide

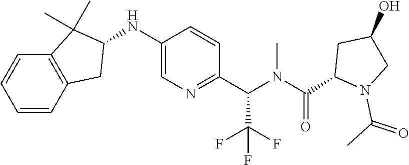 [SMILES: CC(=O)N1C[C@H](O)C[C@H]1C(=O)N(C)[C@@H](c1ccc(N[C@@H]2Cc3ccccc3C2(C)C)cn1)C(F)(F)F]

The title compound (7 mg) was prepared in an analogous manner to Example 16 from Intermediate 125 (0.11 g, 0.18 mmol), TBAF (1M in THF, 0.18 mL, 0.18 mmol) in THF (0.9 mL) at RT for 1 h. Purified by preparative HPLC (Gemini NX-C18, 21×250 mm×5 μm, flow rate: 30 mL/min, water with 50% MeCN isocratic). ¹H NMR (300 MHz; DMSO-d₆) δ: 8.13 (s, 1H), 7.27-7.15 (m, 4H), 7.02 (d, 1H), 6.49 (dd, 1H), 5.01 (dd, 1H), 4.75-4.20 (m, 1H), 4.15-4.00 (m, 1H), 3.93 (dd, 1H), 3.57 (d, 1H), 3.36 (dd, 1H), 3.13 (s, 3H), 2.75 (dd, 1H), 2.37-2.23 (m, 2H), 2.20 (s, 3H), 2.15-1.80 (m, 3H), 1.40 (s, 3H), 1.22 (s, 3H). LCMS (Method M): 505.2 [M+H]⁺.

### Example 221: (1S,3R)—N—((S)-1-(5-(((S)-1,1-Dimethyl-2,3-dihydro-1H-inden-2-yl)amino)pyridin-2-yl)-2,2,2-trifluoroethyl)-N-methyl-3-(methylsulfonamido)cyclopentane-1-carboxamide

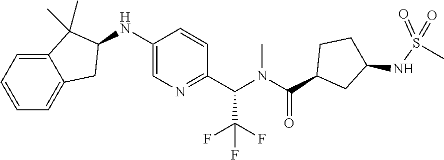 [SMILES: CN(C(=O)[C@H]1CC[C@@H](NS(C)(=O)=O)C1)[C@@H](c1ccc(N[C@H]2Cc3ccccc3C2(C)C)cn1)C(F)(F)F]

The title compound (15 mg) was prepared in an analogous manner to Example 169 from Intermediate 105 (44 mg, 0.10 mmol), triethylamine (40 μL, 0.29 mmol) and methane sulfonyl chloride (13 mg, 0.11 mmol) in DCM (0.6 mL) at RT for 15 min. Purified by preparative HPLC (Gemini NX-C18, 21×250 mm×5 μm, flow rate: 30 mL/min, 0.05% aqueous ammonia with MeCN 60% isocratic for 12 min, ramped to 95% over 0.2 min and held for 1.8 min). ¹H NMR (400 MHz; DMSO-d₆) δ: 8.15 (d, 1H), 7.23-7.05 (m, 7H), 6.40 (dd, 1H), 6.22 (d, 1H), 4.05 (dd, 1H), 3.67-3.59 (m, 1H), 3.23-3.12 (m, 2H), 2.96 (s, 3H), 2.91 (s, 3H), 2.78-2.72 (m, 1H), 2.26-2.24 (m, 1H), 1.93-1.90 (m, 1H), 1.78 (dd, 2H), 1.68-1.47 (m, 2H), 1.33 (s, 3H), 1.10 (s, 3H). LCMS (Method M): 539.3 [M+H]⁺.

### Example 222: (1,3-trans)-N—((S)-1-(5-(((S)-1,1-Dimethyl-2,3-dihydro-1H-inden-2-yl)amino)pyridin-2-yl)-2,2,2-trifluoroethyl)-N-methyl-3-(methylsulfonamido)cyclobutane-1-carboxamide

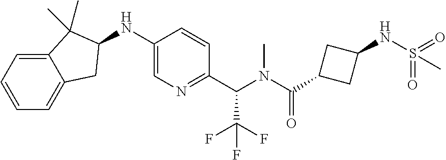 [SMILES: CN(C(=O)[C@H]1C[C@H](NS(C)(=O)=O)C1)[C@@H](c1ccc(N[C@H]2Cc3ccccc3C2(C)C)cn1)C(F)(F)F]

The title compound (26 mg) was prepared in an analogous manner to Example 169 from Intermediate 103 (58 mg, 0.13 mmol), triethylamine (54 μL, 0.39 mmol) and methane sulfonyl chloride (18 mg, 0.16 mmol) in DCM (0.9 mL) at RT for 15 min. Purified by preparative HPLC (Gemini NX-C18, 21×250 mm×5 μm, flow rate: 30 mL/min, 0.05% aqueous ammonia with MeCN 30% to 68% over 6 min, held for 4 min ramped to 95% over 0.1 min and held for 1.9 min). ¹H NMR (400 MHz; DMSO-d₆) δ: 8.15 (d, 1H), 7.51 (dd, 1H), 7.24-7.08 (m, 7H), 6.39 (dd, 1H), 6.23 (d, 1H), 4.05 (dd, 1H), 3.85 (dd, 1H), 3.39-3.34 (m, 1H), 3.20 (dd, 1H), 2.88-2.81 (m, 6H), 2.80-2.70 (m, 2H), 2.32-2.18 (m, 2H), 1.33 (s, 3H), 1.10 (s, 3H). LCMS (Method M): 525.3 [M+H]⁺.

### Example 223: N—((S)-1-(5-(((S)-1,1-Dimethyl-2,3-dihydro-1H-inden-2-yl)amino)pyridin-2-yl)-2,2,2-trifluoroethyl)-N-methyl-1-(methylsulfonyl)piperidine-4-carboxamide

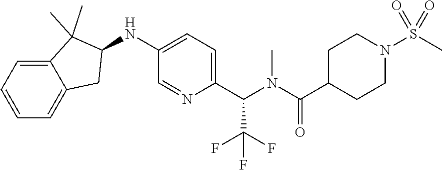 [SMILES: CN(C(=O)C1CCN(S(C)(=O)=O)CC1)[C@@H](c1ccc(N[C@H]2Cc3ccccc3C2(C)C)cn1)C(F)(F)F]

The title compound (26 mg) was prepared in an analogous manner to Example 169 from Intermediate 128 (80 mg, 0.16 mmol), DIPEA (0.11 mL, 0.64 mmol) and methane sulfonyl chloride (18 mg, 0.16 mmol) in THF (0.4 mL) and MeCN (0.4 mL) at RT for 16 h. Purified by preparative HPLC (Kinetex XB-C18, 21×250 mm×5 μm, flow rate: 30 mL/min, 0.1% aqueous formic acid with MeCN 40% to 60% over 6 min, held for 5 min ramped to 95% over 0.2 min and held for 1.8 min). ¹H NMR (300 MHz; DMSO-d₆) δ: 8.15 (s, 1H), 7.30-7.01 (m, 6H), 6.38 (dd, 1H), 6.23 (d, 1H), 4.15-3.95 (m, 1H), 3.59 (d, 2H), 3.20 (dd, 2H), 3.03 (s, 3H), 2.87 (s, 3H), 2.86-2.70 (m, 3H), 1.95-1.68 (m, 2H), 1.62-1.52 (m, 2H), 1.33 (s, 3H), 1.11 (s, 3H). LCMS (Method M): 539.2 [M+H]⁺.

### Example 224: 1-Acetyl-N—((S)-1-(5-(((S)-1,1-dimethyl-2,3-dihydro-1H-inden-2-yl)amino)pyridin-2-yl)-2,2,2-trifluoroethyl)-N-methylpiperidine-4-carboxamide

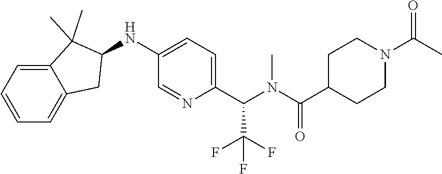 [SMILES: CC(=O)N1CCC(C(=O)N(C)[C@@H](c2ccc(N[C@H]3Cc4ccccc4C3(C)C)cn2)C(F)(F)F)CC1]

The title compound (29 mg) was prepared in an analogous manner to Example 169 from Intermediate 128 (80 mg, 0.16 mmol), DIPEA (0.11 mL, 0.64 mmol) and acetyl chloride (1M in DCM, 0.16 mL, 0.16 mmol) in THF (0.8 mL) at RT for 16 h. Purified by preparative HPLC (Kinetex XB-C18, 21×250 mm×5 μm, flow rate: 30 mL/min, 0.1% aqueous formic acid with MeCN 50% to 55% over 6 min, held for 5 min ramped to 95% over 0.2 min and held for 1.8 min). ¹H NMR (300 MHz; DMSO-d₆) δ: 8.15 (d, 1H), 7.28-7.03 (m, 6H), 6.39 (dd, 1H), 6.22 (d, 1H), 4.37 (d, 1H), 4.05 (dd, 1H), 3.83 (d, 1H), 3.26-3.00 (m, 6H), 2.82-2.64 (m, 2H), 2.62-2.55 (m, 1H), 2.00 (s, 3H), 1.85-1.42 (m, 3H), 1.33 (s, 3H), 1.11 (s, 3H). LCMS (Method M): 503.3 [M+H]⁺.

### Example 225: N—((S)-1-(5-(((S)-1,1-Dimethyl-2,3-dihydro-1H-inden-2-yl)amino)pyridin-2-yl)-2,2,2-trifluoroethyl)-N,1-dimethyl-5-oxopyrrolidine-2-carboxamide—Isomer 1

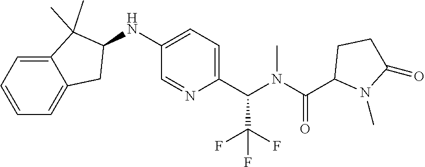 [SMILES: CN1C(=O)CCC1C(=O)N(C)[C@@H](c1ccc(N[C@H]2Cc3ccccc3C2(C)C)cn1)C(F)(F)F]

### Example 226: N—((S)-1-(5-(((S)-1,1-Dimethyl-2,3-dihydro-1H-inden-2-yl)amino)pyridin-2-yl)-2,2,2-trifluoroethyl)-N,1-dimethyl-5-oxopyrrolidine-2-carboxamide—Isomer 2

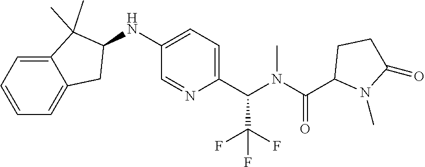 [SMILES: CN1C(=O)CCC1C(=O)N(C)[C@@H](c1ccc(N[C@H]2Cc3ccccc3C2(C)C)cn1)C(F)(F)F]

The title compound was prepared in an analogous manner to Intermediate 3 from Intermediate 82 (50 mg, 0.14 mmol), pyridine (0.6 mL), T3P® (50% in EtOAc, 0.26 mL, 0.43 mmol) and 1-methyl-5-oxo-pyrrolidine-2-carboxylic acid (22 mg, 0.16 mmol, CAS 72442-37-6) at RT for 16 h. Purified by preparative HPLC (Gemini NX-C18, 21×250 mm×5 μm, flow rate: 33 mL/min, 0.1% aqueous formic acid with MeCN 43% isocratic over 11 min, ramped to 95% over 0.2 min and held for 1.8 min) to provide Example 225 (7 mg) as Peak 1, Example 226 (5 mg) as Peak 2. Example 225: ¹H NMR (400 MHz; DMSO-d₆) δ: 8.16 (d, 1H), 7.28-7.10 (m, 6H), 6.43-6.20 (m, 2H), 4.73 (dd, 1H), 4.06 (dd, 1H), 3.19 (dd, 1H), 3.02 (s, 3H), 2.82-2.58 (m, 4H), 2.43-2.30 (m, 1H), 2.22 (dd, 2H), 1.74 (dd, 1H), 1.32 (s, 3H), 1.10 (s, 3H). LCMS (Method M): 475.3 [M+H]⁺. Example 226: ¹H NMR (400 MHz; DMSO-d₆) δ: 8.16 (d, 1H), 7.27-7.08 (m, 6H), 6.40-6.18 (m, 2H), 4.73 (dd, 1H), 4.05 (dd, 1H), 3.19 (dd, 1H), 2.98 (s, 3H), 2.81-2.70 (m, 1H), 2.64 (s, 3H), 2.32-2.15 (m, 3H), 1.76-1.60 (m, 1H), 1.32 (s, 3H), 1.10 (s, 3H). LCMS (Method M): 475.3 [M+H]⁺.

### Example 227: N³-((1S)-1-(5-((1,1-dimethyl-2,3-dihydro-1H-inden-2-yl)amino)pyridin-2-yl)-2,2,2-trifluoroethyl)-N¹,N³-dimethylazetidine-1,3-dicarboxamide—Isomer 1

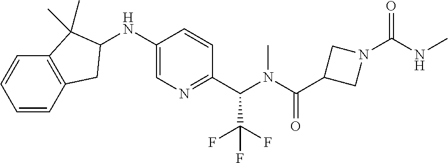 [SMILES: CNC(=O)N1CC(C(=O)N(C)[C@@H](c2ccc(NC3Cc4ccccc4C3(C)C)cn2)C(F)(F)F)C1]

### Example 228: N³-((1S)-1-(5-((1,1-dimethyl-2,3-dihydro-1H-inden-2-yl)amino)pyridin-2-yl)-2,2,2-trifluoroethyl)-N¹,N³-dimethylazetidine-1,3-dicarboxamide—Isomer 2

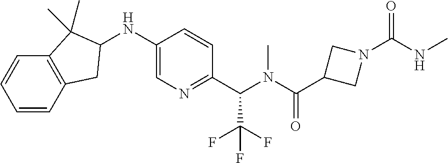 [SMILES: CNC(=O)N1CC(C(=O)N(C)[C@@H](c2ccc(NC3Cc4ccccc4C3(C)C)cn2)C(F)(F)F)C1]

To a stirred solution of Intermediate 130 (0.15 g, 0.26 mmol) and methylamine (1M in THF, 2.48 mL, 2.48 mmol) in THF (12 mL) was added 4-nitrophenyl chloroformate (0.10 g, 0.50 mmol) at 0° C. for 10 min. Triethylamine (0.35 mL, 2.48 mmol) was added then the mixture was stirred at 60° C. for 16 h. Additional methylamine (1M in THF, 4.97 mL, 4.97 mmol) was added and stirred at 60° C. for 6 h. The mixture was poured into water and extracted with EtOAc. The organic layer washed with water, brine, dried over Na₂SO₄, filtered and concentrated under reduced pressure. The crude product was purified by preparative HPLC (Gemini NX-C18, 19×150 mm×5 μm, flow rate: 18 mL/min, 10 mM aqueous NH₄HCO₃ with MeCN 50% to 70% for 7 min, held for 1.4 min, ramped to 100% over 0.2 min and held for 2.9 min). Chiral purification was carried out by preparative SFC (Chiralpak®-AD-H, 30×250 mm×5 μm, temp 30° C., Back Pressure 100 bar, flow rate: 100 g/min, 60% CO₂ with 40% MeOH modifier) to provide Example 227 (12 mg) as Peak 1, Example 228 (16 mg) as Peak 2. Example 227: ¹H NMR (400 MHz; DMSO-d₆) δ: 8.14 (s, 1H), 7.21-7.10 (m, 6H), 6.35-6.21 (m, 3H), 4.03-3.75 (m, 6H), 3.21-3.16 (m, 1H), 2.80-2.71 (m, 4H), 2.53-2.51 (m, 3H), 1.40 (s, 3H), 1.09 (s, 3H). LCMS (Method A): 490.4 [M+H]⁺. Example 228: ¹H NMR (400 MHz; DMSO-d₆) δ: 8.14 (s, 1H), 7.21-7.10 (m, 6H), 6.38-6.20 (m, 3H), 3.94-3.76 (m, 6H), 3.22-3.16 (m, 1H), 2.81-2.70 (m, 4H), 2.53-2.51 (m, 3H), 1.40 (s, 3H), 1.09 (s, 3H). LCMS (Method A): 490.5 [M+H]⁺.

### Example 229: N-((1S)-1-(5-((1,1-Dimethyl-2,3-dihydro-1H-inden-2-yl)amino)pyridin-2-yl)-2,2,2-trifluoroethyl)-N-methyl-6-oxo-5-azaspiro[3.4]octane-2-carboxamide—Isomer 1

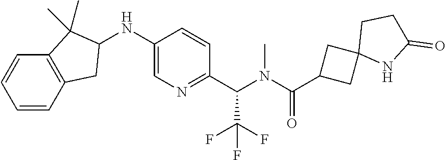 [SMILES: CN(C(=O)C1CC2(CCC(=O)N2)C1)[C@@H](c1ccc(NC2Cc3ccccc3C2(C)C)cn1)C(F)(F)F]

### Example 230: N-((1S)-1-(5-((1,1-Dimethyl-2,3-dihydro-1H-inden-2-yl)amino)pyridin-2-yl)-2,2,2-trifluoroethyl)-N-methyl-6-oxo-5-azaspiro[3.4]octane-2-carboxamide—Isomer 2

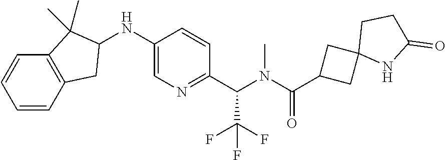 [SMILES: CN(C(=O)C1CC2(CCC(=O)N2)C1)[C@@H](c1ccc(NC2Cc3ccccc3C2(C)C)cn1)C(F)(F)F]

### Example 231: N-((1S)-1-(5-((1,1-Dimethyl-2,3-dihydro-1H-inden-2-yl)amino)pyridin-2-yl)-2,2,2-trifluoroethyl)-N-methyl-6-oxo-5-azaspiro[3.4]octane-2-carboxamide—Isomer 3

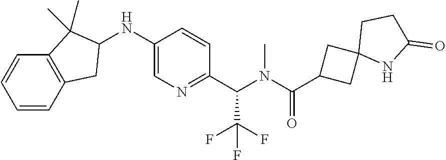 [SMILES: CN(C(=O)C1CC2(CCC(=O)N2)C1)[C@@H](c1ccc(NC2Cc3ccccc3C2(C)C)cn1)C(F)(F)F]

### Example 232: N-((1S)-1-(5-((1,1-Dimethyl-2,3-dihydro-1H-inden-2-yl)amino)pyridin-2-yl)-2,2,2-trifluoroethyl)-N-methyl-6-oxo-5-azaspiro[3.4]octane-2-carboxamide—Isomer 4

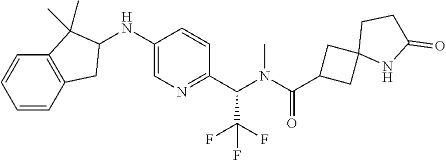 [SMILES: CN(C(=O)C1CC2(CCC(=O)N2)C1)[C@@H](c1ccc(NC2Cc3ccccc3C2(C)C)cn1)C(F)(F)F]

The title compound was prepared in an analogous manner to Intermediate 3 from Intermediate 61 (0.15 g, 0.40 mmol), pyridine (0.5 mL), T3P® (50% in EtOAc, 1.19 mL, 3.99 mmol) and 6-oxo-5-azaspiro[3.4]octane-2-carboxylic acid (0.18 g, 0.80 mmol, CAS 2166848-89-9) at RT for 16 h. Purified by preparative HPLC (X-Select C18, 19×250 mm×5 μm, flow rate: 18 mL/min, 10 mM aqueous NH₄HCO₃ with MeCN 45% to 65% over 9 min, held for 2.4 min, ramped to 100% over 0.1 min and held for 2.6 min). Chiral purification was carried out by preparative SFC (Lux Cellulose-4, 30×250 mm×5 μm, temp 30° C., Back Pressure 100 bar, flow rate: 100 g/min, 60% CO₂ with 40% MeOH modifier) to provide Example 229 (9 mg) as Peak 1, Example 230 (8 mg) as Peak 2, Example 231 (26 mg) as Peak 3, Example 232 (22 mg) as Peak 4. Example 229: ¹H NMR (400 MHz; DMSO-d₆) δ: 8.14-8.13 (m, 1H), 7.24-7.11 (m, 6H), 6.40-6.18 (m, 2H), 4.04 (dd, 1H), 3.36-3.16 (m, 2H), 2.84-2.70 (m, 4H) 2.48-2.30 (m, 4H), 2.17-1.99 (m, 4H) 1.32 (s, 3H), 1.09 (s, 3H).—1H obscured by solvent. LCMS (Method A): 501.4 [M+H]⁺. Example 230: ¹H NMR (400 MHz; DMSO-d₆) δ: 8.16-8.13 (m, 1H), 7.21-7.08 (m, 6H), 6.41-6.19 (m, 2H), 4.04 (dd, 1H), 3.32-3.16 (m, 2H), 2.83-2.71 (m, 4H) 2.44-2.32 (m, 4H), 2.13-1.98 (m, 4H), 1.32 (s, 3H), 1.10 (s, 3H)—1H obscured by solvent. LCMS (Method A): 501.4 [M+H]⁺. Example 231: ¹H NMR (400 MHz; DMSO-d₆) δ: 8.14-8.13 (m, 1H), 7.22-7.08 (m, 6H), 6.37-6.19 (m, 2H), 4.07-4.00 (m, 1H), 3.32-3.14 (m, 2H), 2.89-2.71 (m, 4H) 2.34-2.15 (m, 8H), 1.32 (s, 3H), 1.10 (s, 3H)—1H obscured by solvent. LCMS (Method A): 501.4 [M+H]⁺. Example 232: ¹H NMR (400 MHz; DMSO-d₆) δ: 8.14-8.13 (m, 1H), 7.22-7.09 (m, 6H), 6.37-6.19 (m, 2H), 4.07-4.00 (m, 1H), 3.32-3.15 (m, 2H), 2.89-2.73 (m, 4H) 2.33-2.15 (m, 8H), 1.32 (s, 3H), 1.09 (s, 3H)—1H obscured by solvent. LCMS (Method A): 501.4 [M+H]⁺.

### Example 233: N—((S)-1-(5-(((S)-1,1-dimethyl-2,3-dihydro-1H-inden-2-yl)amino)pyridin-2-yl)-2,2,2-trifluoroethyl)-N-methyl-3-(methylsulfonamido)bicyclo[1.1.1]pentane-1-carboxamide

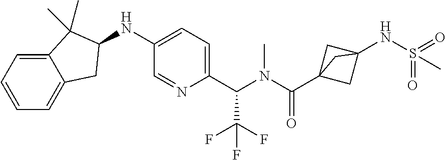 [SMILES: CN(C(=O)C12CC(NS(C)(=O)=O)(C1)C2)[C@@H](c1ccc(N[C@H]2Cc3ccccc3C2(C)C)cn1)C(F)(F)F]

The title compound (54 mg) was prepared in an analogous manner to Example 169 from Intermediate 92 (0.21 g, 0.24 mmol), triethylamine (0.10 mL, 0.72 mmol) and methane sulfonyl chloride (34 mg, 0.30 mmol) in DCM (1.6 mL) at RT for 30 min. Purified by preparative HPLC (Gemini NX-C18, 21×250 mm×5 μm, flow rate: 30 mL/min, 0.1% aqueous formic acid with MeCN 50% to 55% over 6 min, held for 9 min, ramped to 95% over 0.5 min and held for 1.5 min). ¹H NMR (400 MHz; DMSO-d₆) δ: 8.16-8.10 (m, 1H), 7.30-7.01 (m, 6H), 6.45-6.13 (m, 2H), 4.05 (dd, 1H), 3.19 (dd, 1H), 3.10-2.90 (m, 5H), 2.76-2.66 (m, 2H), 2.40-2.26 (m, 6H), 1.33 (s, 3H), 1.11 (s, 3H)—1H obscured by solvent. LCMS (Method M): 537.3 [M+H]⁺.

### Example 234: N—((S)-1-(5-(((S)-1,1-Dimethyl-2,3-dihydro-1H-inden-2-yl)amino)pyridin-2-yl)-2,2,2-trifluoroethyl)-2-(1,1-dioxidothiomorpholino)-N-methylacetamide

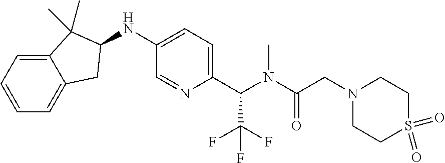 [SMILES: CN(C(=O)CN1CCS(=O)(=O)CC1)[C@@H](c1ccc(N[C@H]2Cc3ccccc3C2(C)C)cn1)C(F)(F)F]

The title compound (32 mg) was prepared in an analogous manner to Intermediate 3 from Intermediate 82 (50 mg, 0.14 mmol), pyridine (0.7 mL), T3P® (50% MeTHF, 0.26 mL, 0.43 mmol) and 2-(1,1-dioxidothiomorpholino)acetic acid (30 mg, 0.16 mmol, CAS 155480-08-3) at RT for 17 h. Purified by preparative HPLC (Gemini NX-C18, 21×250 mm×5 μm, flow rate: 30 mL/min, 0.05% aqueous ammonia with MeCN 55% to 60% over 6 min, held for 5 min, ramped to 95% over 0.2 min and held for 1.8 min). ¹H NMR (300 MHz; DMSO-d₆) δ: 8.15 (d, 1H), 7.27-7.19 (m, 3H), 7.19-7.09 (m, 3H), 6.34 (dd, 1H), 6.23 (d, 1H), 4.06 (dd, 1H), 3.60 (d, 1H), 3.19 (dd, 1H), 3.15-2.95 (m, 11H), 2.80-2.71 (m, 2H), 1.33 (s, 3H), 1.10 (s, 3H). LCMS (Method M): 525.2 [M+H]⁺.

### Example 235: 1-Acetyl-N-((1S)-1-(5-((1,1-dimethyl-2,3-dihydro-1H-inden-2-yl)amino)pyridin-2-yl)-2,2,2-trifluoroethyl)-N-methyl-1-azaspiro[3.3]heptane-6-carboxamide—Isomer 1

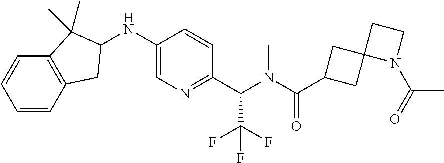 [SMILES: CC(=O)N1CCC12CC(C(=O)N(C)[C@@H](c1ccc(NC3Cc4ccccc4C3(C)C)cn1)C(F)(F)F)C2]

### Example 236: 1-Acetyl-N-((1S)-1-(5-((1,1-dimethyl-2,3-dihydro-1H-inden-2-yl)amino)pyridin-2-yl)-2,2,2-trifluoroethyl)-N-methyl-1-azaspiro[3.3]heptane-6-carboxamide—Isomer 2

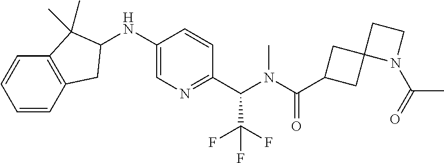 [SMILES: CC(=O)N1CCC12CC(C(=O)N(C)[C@@H](c1ccc(NC3Cc4ccccc4C3(C)C)cn1)C(F)(F)F)C2]

The title compound was prepared in an analogous manner to Intermediate 3 from Intermediate 138 (0.25 g, 0.49 mmol), pyridine (0.12 mL, 1.48 mmol), AcOH (34 μL, 0.59 mmol), T3P® (50% in EtOAc, 1.46 mL, 4.92 mmol) at RT for 16 h. Purified by preparative HPLC (XBridge C18, 19×250 mm×5 μm, flow rate: 18 mL/min, 10 mM aqueous NH₄HCO₃ with MeCN 50% to 65% over 6 min, held for 3.75 min, ramped to 100% over 0.1 min and held for 4.2 min). Chiral purification was carried out by preparative SFC (Lux Cellulose-4, 30×250 mm×5 μm, temp 30° C., Back Pressure 100 bar, flow rate: 100 g/min, 60% CO₂ with 40% MeOH modifier) to provide Example 235 (9 mg) as Peak 1, Example 236 (9 mg) as Peak 2. Example 235: ¹H NMR (400 MHz; DMSO-d₆) δ: 8.14 (d, 1H), 7.21-7.08 (m, 6H), 6.39-6.19 (m, 2H), 4.04 (dd, 1H), 3.90 (dd, 1H), 3.60-3.48 (m, 2H), 3.22 (dd, 1H), 3.03-2.71 (m, 6H), 2.49-2.15 (m, 4H), 1.72 (s, 3H), 1.32 (s, 3H), 1.10 (s, 3H). LCMS (Method A): 515.5 [M+H]⁺. Example 236: ¹H NMR (400 MHz; DMSO-d₆) δ: 8.13 (d, 1H), 7.21-7.07 (m, 6H), 6.37-6.18 (m, 2H), 4.03 (dd, 1H), 3.90 (dd, 1H), 3.52-3.50 (m, 2H), 3.18 (dd, 1H), 3.10-2.51 (m, 6H), 2.35-2.10 (m, 4H), 1.72 (s, 3H), 1.32 (d, 3H), 1.09 (s, 3H). LCMS (Method Q): 515.5 [M+H]⁺.

### Example 237: N-((1S)-1-(5-((4-Bromo-2,3-dihydro-1H-inden-2-yl)amino)pyridin-2-yl)-2,2,2-trifluoroethyl)-N-methyltetrahydro-2H-thiopyran-4-carboxamide 1,1-dioxide

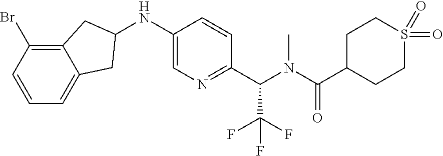 [SMILES: CN(C(=O)C1CCS(=O)(=O)CC1)[C@@H](c1ccc(NC2Cc3cccc(Br)c3C2)cn1)C(F)(F)F]

The title compound (5 mg) was prepared in an analogous manner to Intermediate 26 from Intermediate 90 (0.25 g, 0.55 mmol), 4-bromo-2,3-dihydro-1H-inden-2-amine (0.17 g, 0.61 mmol, CAS 1196157-02-4), Cs₂CO₃ (0.54 g, 1.66 mmol), Pd₂(dba)₃ (51 mg, 0.06 mmol) and X-Phos (53 mg, 0.11 mmol) in toluene (5 mL) at 110° C. for 2 h. Purified by preparative HPLC (XBridge C18, 19×250 mm×5 μm, flow rate: 18 mL/min, 10 mM aqueous NH₄HCO₃ with MeCN 35% to 60% over 8 min, held for 6.5 min, ramped to 100% over 0.1 min and held for 5.4 min). ¹H NMR (400 MHz; DMSO-d₆) δ: 8.05-8.02 (m, 1H), 7.38 (d, 1H), 7.26 (d, 1H), 7.16-7.01 (m, 3H), 6.58-6.04 (m, 2H), 4.28 (dd, 1H), 3.47-3.36 (m, 2H), 3.25-3.12 (m, 5H), 3.09 (s, 3H), 2.96 (dd, 1H), 2.82 (dd, 1H), 2.25-1.92 (m, 4H). LCMS (Method A): 562.3 [M+H]⁺.

### Example 238: N⁶-((1S)-1-(5-((1,1-Dimethyl-2,3-dihydro-1H-inden-2-yl)amino)pyridin-2-yl)-2,2,2-trifluoroethyl)-N¹,N⁶-dimethyl-1-azaspiro[3.3]heptane-1,6-dicarboxamide—Isomer 1

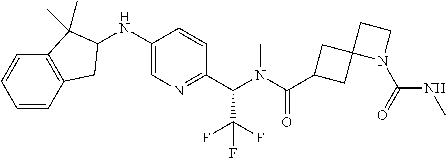 [SMILES: CNC(=O)N1CCC12CC(C(=O)N(C)[C@@H](c1ccc(NC3Cc4ccccc4C3(C)C)cn1)C(F)(F)F)C2]

### Example 239: N⁶-((1S)-1-(5-((1,1-Dimethyl-2,3-dihydro-1H-inden-2-yl)amino)pyridin-2-yl)-2,2,2-trifluoroethyl)-N,N-dimethyl-1-azaspiro[3.3]heptane-1,6-dicarboxamide—Isomer 2

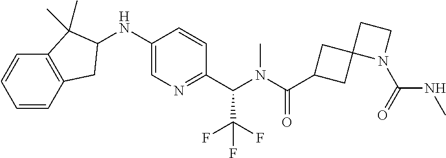 [SMILES: CNC(=O)N1CCC12CC(C(=O)N(C)[C@@H](c1ccc(NC3Cc4ccccc4C3(C)C)cn1)C(F)(F)F)C2]

The title compounds were prepared in an analogous manner to Example 227 using Example 154 (0.35 g, 0.58 mmol), methylamine (1M in THF, 5.78 mL, 5.78 mmol), 4-nitrophenyl chloroformate (0.18 g, 0.87 mmol) and triethylamine (0.40 mL, 2.89 mmol) in THF (5 mL) at 60° C. for 16 h in a sealed tube. Additional methylamine hydrochloride (0.39 mL, 5.77 mmol) and triethylamine (0.40 mL, 2.89 mmol) was added and stirred at 60° C. for 16 h. The crude product was purified by preparative HPLC (X-Select CHS C18, 19×150 mm×5 μm, flow rate: 18 mL/min, 10 mM aqueous NH₄HCO₃ with MeCN 45% to 60% for 6 min, held for 5.5 min, ramped to 100% over 0.1 min and held for 4.4 min). Chiral purification was carried out by preparative SFC (Lux Cellulose-4, 30×250 mm×5 μm, temp 30° C., Back Pressure 100 bar, flow rate: 100 g/min, 75% CO₂ with 25% MeOH modifier) to provide Example 238 (19 mg) as Peak 1, Example 239 (21 mg) as Peak 2. Example 238: ¹H NMR (400 MHz; DMSO-d₆) δ: 8.13 (d, 1H), 7.22-7.07 (m, 6H), 6.38-6.01 (m, 3H), 4.04 (dd, 1H), 3.62 (dd, 2H), 3.44-3.40 (m, 1H), 3.19 (dd, 1H), 2.99-2.69 (m, 6H), 2.56-2.53 (m, 3H), 2.31-2.17 (m, 2H), 2.16-2.10 (m, 2H), 1.31 (s, 3H), 1.09 (s, 3H). LCMS (Method Q): 530.4 [M+H]⁺. Example 239: ¹H NMR (400 MHz; DMSO-d₆) δ: 8.13 (d, 1H), 7.21-7.06 (m, 6H), 6.40-5.95 (m, 3H), 4.04 (dd, 1H), 3.60 (dd, 2H), 3.41-3.31 (m, 1H), 3.18 (dd, 1H), 2.99-2.71 (m, 6H), 2.55-2.50 (m, 3H), 2.31-2.15 (m, 2H), 2.14-2.09 (m, 2H), 1.31 (s, 3H), 1.09 (s, 3H). LCMS (Method Q): 530.4 [M+H]⁺.

### Example 240: (1,3-cis)-N¹—((S)-1-(5-(((S)-1,1-Dimethyl-2,3-dihydro-1H-inden-2-yl)amino)pyridin-2-yl)-2,2,2-trifluoroethyl)-N¹,N³-dimethylcyclobutane-1,3-dicarboxamide

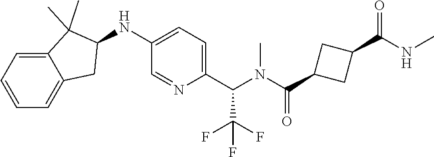 [SMILES: CNC(=O)[C@H]1C[C@@H](C(=O)N(C)[C@@H](c2ccc(N[C@H]3Cc4ccccc4C3(C)C)cn2)C(F)(F)F)C1]

The title compound (11 mg) was prepared in an analogous manner to Intermediate 3 from Intermediate 82 (40 mg, 0.12 mmol), pyridine (2.3 mL), T3P® (50% MeTHF, 0.42 mL, 0.69 mmol) and Intermediate 132 (23 mg, 0.15 mmol) at RT for 16 h. Purified by preparative HPLC (Gemini NX-C18, 21×250 mm×5 μm, flow rate: 30 mL/min, 0.1% aqueous formic acid with MeCN 50% to 65% over 6 min, held for 3 min, ramped to 95% over 0.2 min and held for 1.8 min). ¹H NMR (400 MHz; DMSO-d₆) δ: 8.14 (d, 1H), 7.67 (d, 1H), 7.25-7.10 (m, 5H), 7.08 (d, 1H), 6.33 (dd, 1H), 6.21 (d, 1H), 4.04 (dd, 1H), 3.19 (dd, 1H), (dd, 1H), 2.85 (s, 3H), 2.80-2.69 (m, 2H), 2.60-2.49 (m, 3H), 2.36-2.15 (m, 4H), 1.32 (s, 3H), 1.09 (s, 3H). LCMS (Method M): 489.3 [M+H]⁺.

### Example 241: (1,3-cis)-N—((S)-1-(5-(((S)-1,1-Dimethyl-2,3-dihydro-1H-inden-2-yl)amino)pyridin-2-yl)-2,2,2-trifluoroethyl)-N-methyl-3-(methylsulfonamido)cyclobutane-1-carboxamide

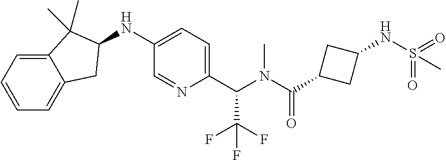 [SMILES: CN(C(=O)[C@H]1C[C@@H](NS(C)(=O)=O)C1)[C@@H](c1ccc(N[C@H]2Cc3ccccc3C2(C)C)cn1)C(F)(F)F]

The title compound (29 mg) was prepared in an analogous manner to Example 169 from Intermediate 126 (0.22 g, 0.42 mmol), triethylamine (0.18 mL, 1.28 mmol) and methane sulfonyl chloride (61 mg, 0.53 mmol) in DCM (1.6 mL) at RT for 30 min. Purified by preparative HPLC (Gemini NX-C18, 21×250 mm×5 μm, flow rate: 30 mL/min, 0.1% aqueous formic acid with MeCN 50% to 60% over 6 min, held for 4 min, ramped to 95% over 0.2 min and held for 1.8 min). ¹H NMR (400 MHz; DMSO-d₆) δ: 8.14 (d, 1H), 7.41 (d, 1H), 7.26-7.03 (m, 6H), 6.33 (dd, 1H), 6.22 (d, 1H), 4.04 (dd, 1H), 3.70 (dd, 1H), 3.28-3.02 (m, 2H), 2.87 (s, 3H), 2.86-2.82 (m, 3H), 2.79-2.69 (m, 2H), 2.60-2.53 (m, 1H), 2.17-2.04 (m, 2H), 1.31 (s, 3H), 1.09 (s, 3H). LCMS (Method M): 525.3 [M+H]⁺.

### Example 242: N-((1S)-1-(5-((1,1-Dimethyl-2,3-dihydro-1H-inden-2-yl)amino)pyridin-2-yl)-2,2,2-trifluoroethyl)-1-(2-methoxyethyl)-N-methyl-1-azaspiro[3.3]heptane-6-carboxamide—Isomer 1

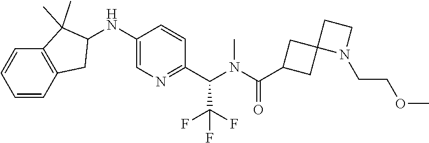 [SMILES: COCCN1CCC12CC(C(=O)N(C)[C@@H](c1ccc(NC3Cc4ccccc4C3(C)C)cn1)C(F)(F)F)C2]

### Example 243: N-((1S)-1-(5-((1,1-Dimethyl-2,3-dihydro-1H-inden-2-yl)amino)pyridin-2-yl)-2,2,2-trifluoroethyl)-1-(2-methoxyethyl)-N-methyl-1-azaspiro[3.3]heptane-6-carboxamide—Isomer 2

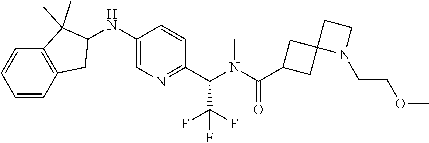 [SMILES: COCCN1CCC12CC(C(=O)N(C)[C@@H](c1ccc(NC3Cc4ccccc4C3(C)C)cn1)C(F)(F)F)C2]

To a stirred solution of Intermediate 138 (0.30 g, 0.49 mmol) and 1-bromo-2-methoxyethane (50 μL, 0.53 mmol) in DMF (6 mL) was added DIPEA (0.25 mL, 1.45 mmol) then the mixture was stirred at 70° C. for 16 h. The mixture was poured into water and extracted with EtOAc. The organic layer washed with water, brine, dried over Na₂SO₄, filtered and concentrated under reduced pressure. The crude was purified by preparative HPLC (XBridge C18, 19×250 mm×5 μm, flow rate: 18 mL/min, 10 mM aqueous NH₄HCO₃ with MeCN 20% to 60% for 6 min, held for 10 min, ramped to 100% over 0.1 min and held for 2.9 min). Chiral purification was carried out by preparative SFC (Lux Cellulose-2, 30×250 mm×5 μm, temp 30° C., Back Pressure 100 bar, flow rate: 100 g/min, 60% CO₂ with 40% (10 mM NH₄HCO₃ in MeOH) modifier) to provide Example 242 (12 mg) as Peak 1, Example 243 (16 mg) as Peak 2. Example 242: ¹H NMR (400 MHz; DMSO-d₆) δ: 8.14 (d, 1H), 7.22-7.06 (m, 6H), 6.40-6.18 (m, 2H), 4.03 (dd, 1H), 3.29-3.14 (m, 7H), 2.97 (dd, 2H), 2.82 (s, 3H), 2.76-2.54 (m, 4H), 2.49-2.44 (m, 1H), 2.21-2.10 (m, 2H), 2.00-1.93 (m, 2H), 1.32 (s, 3H), 1.10 (s, 3H). LCMS (Method A): 531.5 [M+H]⁺. Example 243: ¹H NMR (400 MHz; DMSO-d₆) δ: 8.14 (d, 1H), 7.20-7.05 (m, 6H), 6.40-6.18 (m, 2H), 4.04 (dd, 1H), 3.24-3.14 (m, 7H), 2.97 (dd, 2H), 2.77 (s, 3H), 2.63-2.55 (m, 5H), 2.21-2.12 (m, 2H), 2.01-1.93 (m, 2H), 1.32 (s, 3H), 1.10 (s, 3H). LCMS (Method A): 531.5 [M+H]⁺.

### Example 244: (2S,4R)—N—((S)-1-(5-(((S)-1,1-Dimethyl-2,3-dihydro-1H-inden-2-yl)amino)pyridin-2-yl)-2,2,2-trifluoroethyl)-4-hydroxy-N-methyl-1-(methylsulfonyl)pyrrolidine-2-carboxamide

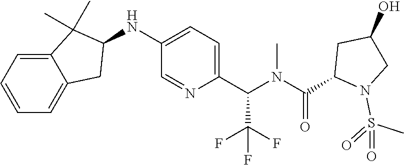 [SMILES: CN(C(=O)[C@@H]1C[C@@H](O)CN1S(C)(=O)=O)[C@@H](c1ccc(N[C@H]2Cc3ccccc3C2(C)C)cn1)C(F)(F)F]

The title compound (50 mg) was prepared in an analogous manner to Example 169 from Intermediate 135 (0.17 g, 0.31 mmol), triethylamine (0.17 mL, 1.24 mmol) and methane sulfonyl chloride (24 μL, 0.31 mmol) in DCM (1.6 mL) at RT for 16 h. Purified by preparative HPLC (Gemini NX-C18, 21×250 mm×5 μm, flow rate: 30 mL/min, water with MeCN 47% to 65% over 6 min, held for 5 min, ramped to 95% over 0.2 min and held for 1.8 min). ¹H NMR (400 MHz; DMSO-d₆) δ: 8.13 (d, 1H), 7.22-7.10 (m, 5H), 7.04 (d, 1H), 6.34 (dd, 1H), 6.22 (d, 1H), 5.22 (d, 1H), 4.84 (dd, 1H), 4.40-4.30 (m, 1H), 4.05 (dd, 1H), 3.46 (dd, 1H), 3.33-3.30 (m, 1H), 3.18 (dd, 1H), 2.99 (s, 3H), 2.94 (s, 3H), 2.79-2.69 (m, 1H), 2.28 (dd, 1H), 1.85 (ddd, 1H), 1.31 (s, 3H), 1.09 (s, 3H). LCMS (Method M): 541.3 [M+H]⁺.

### Example 245: N-((1S)-1-(5-((1,1-Dimethyl-2,3-dihydro-1H-inden-2-yl)amino)pyridin-2-yl)-2,2,2-trifluoroethyl)-N-methyl-2-(2-oxoimidazolidin-1-yl)acetamide—Isomer 1

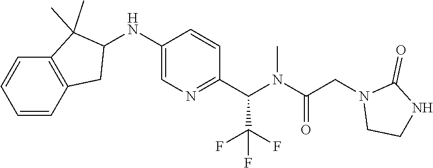 [SMILES: CN(C(=O)CN1CCNC1=O)[C@@H](c1ccc(NC2Cc3ccccc3C2(C)C)cn1)C(F)(F)F]

### Example 246: N-((1S)-1-(5-((1,1-Dimethyl-2,3-dihydro-1H-inden-2-yl)amino)pyridin-2-yl)-2,2,2-trifluoroethyl)-N-methyl-2-(2-oxoimidazolidin-1-yl)acetamide—Isomer 2

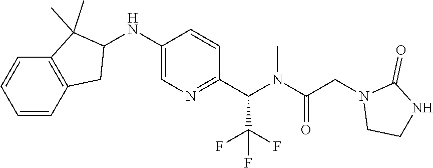 [SMILES: CN(C(=O)CN1CCNC1=O)[C@@H](c1ccc(NC2Cc3ccccc3C2(C)C)cn1)C(F)(F)F]

The title compound was prepared in an analogous manner to Intermediate 3 from Intermediate 61 (0.12 g, 0.33 mmol), pyridine (0.2 mL), 2-(2-oxoimidazolidin-1-yl)acetic acid (59 mg, 0.39 mmol, CAS 87219-22-5), T3P® (50% in EtOAc, 0.97 mL, 3.26 mmol) at RT for 16 h. Purified by preparative HPLC (Gemini NX-C18, 21×250 mm×5 μm, flow rate: 18 mL/min, 5 mM aqueous NH₄HCO₃ with MeCN 50% to 53% over 8 min, held for 5.3 min, ramped to 100% over 0.2 min and held for 2 min). Chiral purification was carried out by preparative SFC (Chiralpak®-AD-H, 30×250 mm×5 μm, temp 30° C., Back Pressure 100 bar, flow rate: 100 g/min, 60% CO₂ with 40% MeOH modifier) to provide Example 245 (11 mg) as Peak 1, Example 246 (11 mg) as Peak 2. Example 245: ¹H NMR (400 MHz; DMSO-d₆) δ: 8.16 (d, 1H), 7.20-7.10 (m, 6H), 6.46-6.20 (m, 3H), 4.22-3.93 (m, 3H), 3.44-3.42 (m, 2H), 3.32-3.16 (m, 3H), 2.96 (s, 3H), 2.76-2.71 (m, 1H), 1.32 (s, 3H), 1.10 (s, 3H). LCMS (Method Q): 476.4 [M+H]⁺. Example 246: ¹H NMR (400 MHz; DMSO-d₆) δ: 8.16 (d, 1H), 7.20-7.10 (m, 6H), 6.46-6.21 (m, 3H), 4.22-3.93 (m, 3H), 3.44-3.42 (m, 2H), 3.32-3.16 (m, 3H), 2.96 (s, 3H), 2.76-2.71 (m, 1H), 1.32 (s, 3H), 1.10 (s, 3H). LCMS (Method Q): 476.4 [M+H]⁺.

### Example 247: (1,3-trans)-N—((S)-1-(5-(((S)-1,1-Dimethyl-2,3-dihydro-1H-inden-2-yl)amino)pyridin-2-yl)-2,2,2-trifluoroethyl)-N¹,N³-dimethylcyclobutane-1,3-dicarboxamide

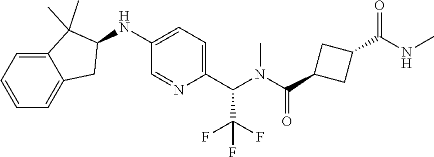 [SMILES: CNC(=O)[C@H]1C[C@H](C(=O)N(C)[C@@H](c2ccc(N[C@H]3Cc4ccccc4C3(C)C)cn2)C(F)(F)F)C1]

The title compound (25 mg) was prepared in an analogous manner to Intermediate 3 from Intermediate 82 (0.12 g, 0.34 mmol), pyridine (2.3 mL), T3P® (50% MeTHF, 1.23 mL, 2.06 mmol) and Intermediate 137 (70 mg, 0.45 mmol) at RT for 16 h. Purified by preparative HPLC (Gemini NX-C18, 21×250 mm×5 μm, flow rate: 30 mL/min, 0.1% aqueous formic acid with MeCN 40% to 55% over 6 min, held for 5 min, ramped to 95% over 0.2 min and held for 1.8 min). ¹H NMR (400 MHz; DMSO-d₆) δ: 8.14 (d, 1H), 7.69 (d, 1H), 7.29-7.04 (m, 6H), 6.37 (dd, 1H), 6.21 (d, 1H), 4.04 (dd, 1H), 3.48-3.37 (m, 1H), 3.19 (dd, 1H), 2.94-2.90 (m, 1H), 2.81 (s, 3H), 2.79-2.69 (m, 1H), 2.57 (d, 3H), 2.43-2.21 (m, 4H), 1.32 (s, 3H), 1.09 (s, 3H). LCMS (Method M): 489.5 [M+H]⁺.

### Example 248: N-((1S)-1-(5-((1,1-Dimethyl-2,3-dihydro-1H-inden-2-yl)amino)pyridin-2-yl)-2,2,2-trifluoroethyl)-1-(2-hydroxyethyl)-N-methyl-1-azaspiro[3.3]heptane-6-carboxamide—Isomer 1

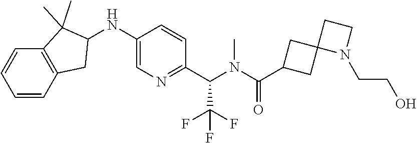 [SMILES: CN(C(=O)C1CC2(CCN2CCO)C1)[C@@H](c1ccc(NC2Cc3ccccc3C2(C)C)cn1)C(F)(F)F]

### Example 249: N-((1S)-1-(5-((1,1-Dimethyl-2,3-dihydro-1H-inden-2-yl)amino)pyridin-2-yl)-2,2,2-trifluoroethyl)-1-(2-hydroxyethyl)-N-methyl-1-azaspiro[3.3]heptane-6-carboxamide—Isomer 2

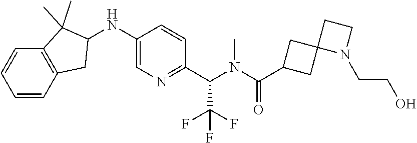 [SMILES: CN(C(=O)C1CC2(CCN2CCO)C1)[C@@H](c1ccc(NC2Cc3ccccc3C2(C)C)cn1)C(F)(F)F]

To a stirred solution of Intermediate 139 (0.27 g, 0.34 mmol) in diethyl ether (3.5 mL) was added 2M HCl in diethyl ether (0.47 mL) and stirred at RT for 2 h. The mixture was concentrated under reduced pressure. The crude product was purified by preparative HPLC (XBridge-C18, 19×250 mm×5 μm, flow rate: 18 mL/min, 10 mM aqueous NH₄HCO₃ with MeCN 40% to 60% over 9 min, held for 2.5 min, ramped to 100% over 0.5 min and held for 4 min). Chiral purification was carried out by preparative SFC (Lux Cellulose-4, 30×250 mm×5 μm, temp 30° C., Back Pressure 100 bar, flow rate: 100 g/min, 65% CO₂ with 35% MeOH modifier) to provide Example 248 (22 mg) as Peak 1, Example 249 (25 mg) as Peak 2. Example 248: ¹H NMR (400 MHz; DMSO-d₆) δ: 8.14 (d, 1H), 7.21-7.06 (m, 6H), 6.41-6.16 (m, 2H), 4.38 (dd, 1H), 4.04 (dd, 1H), 3.39-3.35 (m, 2H), 3.22-3.17 (m, 2H), 2.99-2.96 (m, 2H), 2.82 (s, 3H), 2.77-2.71 (m, 1H), 2.62-2.51 (m, 2H), 2.48-2.45 (m, 2H), 2.19-2.13 (d, 2H), 2.00-1.94 (m, 2H), 1.32 (s, 3H), 1.10 (s, 3H). LCMS (Method Q): 517.5 [M+H]⁺. Example 249: ¹H NMR (400 MHz; DMSO-d₆) δ: 8.14 (d, 1H), 7.21-7.06 (m, 6H), 6.41-6.17 (m, 2H), 4.39-4.36 (m, 1H), 4.04 (dd, 1H), 3.42-3.38 (m, 2H), 3.32-3.15 (m, 2H), 2.99-2.96 (d, 2H), 2.77 (s, 3H), 2.76-2.52 (m, 3H), 2.50-2.46 (m, 2H), 2.19-2.11 (m, 2H), 2.01-1.94 (m, 2H), 1.32 (s, 3H), 1.10 (s, 3H). LCMS (Method Q): 517.5 [M+H]⁺.

### Example 250: N—((S)-1-(5-(((S)-1,1-Dimethyl-2,3-dihydro-1H-inden-2-yl)amino)pyridin-2-yl)-2,2,2-trifluoroethyl)-N,1-dimethyl-2-oxopiperidine-4-carboxamide—Isomer 1

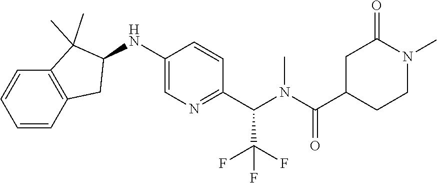 [SMILES: CN1CCC(C(=O)N(C)[C@@H](c2ccc(N[C@H]3Cc4ccccc4C3(C)C)cn2)C(F)(F)F)CC1=O]

### Example 251: N—((S)-1-(5-(((S)-1,1-Dimethyl-2,3-dihydro-1H-inden-2-yl)amino)pyridin-2-yl)-2,2,2-trifluoroethyl)-N,1-dimethyl-2-oxopiperidine-4-carboxamide—Isomer 2

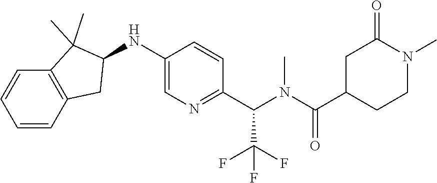 [SMILES: CN1CCC(C(=O)N(C)[C@@H](c2ccc(N[C@H]3Cc4ccccc4C3(C)C)cn2)C(F)(F)F)CC1=O]

The title compound was prepared in an analogous manner to Intermediate 3 from Intermediate 82 (0.12 g, 0.34 mmol), pyridine (1.7 mL), 1-methyl-2-oxopiperidine-4-carboxylic acid (60 mg, 0.38 mmol, CAS 1000932-09-1), T3P® (50% in MeTHF, 0.65 mL, 1.02 mmol) at RT for 16 h. Purified by preparative SFC (Lux Cellulose-4, 30×250 mm×5 μm, temp 30° C., Back Pressure 100 bar, flow rate: 100 g/min, 65% CO₂ with 35% (10 mM NH₄HCO₃ in MeOH) modifier) to provide Example 250 (13 mg) as Peak 1, Example 252 (7 mg) as Peak 2. Example 250: ¹H NMR (400 MHz; DMSO-d₆) δ: 8.17-8.14 (m, 1H), 7.23-7.09 (m, 6H), 6.42-6.19 (m, 2H), 4.05 (dd, 1H), 3.29-3.16 (m, 4H), 3.01 (s, 3H), 2.81-2.71 (m, 4H), 2.37-2.25 (m, 2H), 2.00-1.96 (m, 1H), 1.82-1.78 (m, 1H), 1.32 (s, 3H), 1.10 (s, 3H). LCMS (Method Q): 489.5 [M+H]⁺. Example 251: ¹H NMR (400 MHz; DMSO-d₆) δ: 8.16-8.14 (m, 1H), 7.21-7.08 (m, 6H), 6.40-6.19 (m, 2H), 4.04 (dd, 1H), 3.32-3.16 (m, 4H), 3.00 (s, 3H), 2.82-2.71 (m, 4H), 2.33-2.24 (m, 2H), 1.92-1.85 (m, 1H), 1.78-1.73 (m, 1H), 1.32 (s, 3H), 1.10 (s, 3H). LCMS (Method Q): 489.4 [M+H]⁺.

### Example 252: N—((S)-1-(5-(((S)-1,1-Dimethyl-2,3-dihydro-1H-inden-2-yl)amino)pyridin-2-yl)-2,2,2-trifluoroethyl)-N,1-dimethyl-6-oxopiperidine-3-carboxamide—Isomer 1

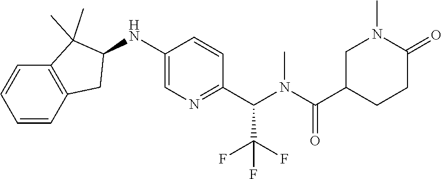 [SMILES: CN1CC(C(=O)N(C)[C@@H](c2ccc(N[C@H]3Cc4ccccc4C3(C)C)cn2)C(F)(F)F)CCC1=O]

### Example 253: N—((S)-1-(5-(((S)-1,1-Dimethyl-2,3-dihydro-1H-inden-2-yl)amino)pyridin-2-yl)-2,2,2-trifluoroethyl)-N,1-dimethyl-6-oxopiperidine-3-carboxamide—Isomer 2

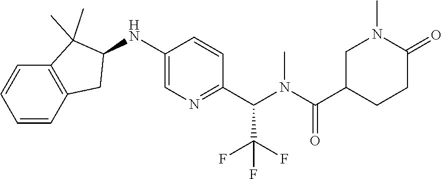 [SMILES: CN1CC(C(=O)N(C)[C@@H](c2ccc(N[C@H]3Cc4ccccc4C3(C)C)cn2)C(F)(F)F)CCC1=O]

The title compound was prepared in an analogous manner to Intermediate 3 from Intermediate 82 (0.10 g, 0.34 mmol), pyridine (1.7 mL), 1-methyl-6-oxopiperidine-3-carboxylic acid (51 mg, 0.38 mmol, CAS 22540-51-8), T3P® (50% in MeTHF, 0.55 mL, 0.87 mmol) at RT for 16 h. Purified by preparative HPLC (Genesis-NX-C18, 21×250 mm×5 μm, flow rate: 30 mL/min, 01% aqueous formic acid with MeCN 40% to 50% over 6 min, held for 9 min, ramped to 95% over 0.2 min and held for 1.8 min). Chiral purification by preparative SFC (Lux Cellulose-4, 30×250 mm×5 μm, temp 30° C., Back Pressure 100 bar, flow rate: 100 g/min, 65% CO₂ with 35% (10 mM NH₄HCO₃ in MeOH) modifier) to provide Example 252 (3 mg) as Peak 1, Example 253 (4 mg) as Peak 2. Example 252: ¹H NMR (400 MHz; DMSO-d₆) δ: 8.17-8.14 (m, 1H), 7.23-7.08 (m, 6H), 6.40-6.13 (m, 2H), 4.05 (dd, 1H), 3.40-3.32 (m, 2H), 3.27-3.16 (m, 2H), 3.04 (s, 3H), 2.83-2.71 (m, 4H), 2.30-2.24 (m, 2H), 1.87-1.76 (m, 2H), 1.32 (s, 3H), 1.10 (s, 3H). LCMS (Method Q): 489.5 [M+H]⁺. Example 253: ¹H NMR (400 MHz; DMSO-d₆) δ: 8.17-8.15 (m, 1H), 7.20-7.10 (m, 6H), 6.42-6.09 (m, 2H), 4.06 (dd, 1H), 3.46-3.32 (m, 2H), 3.18-3.16 (m, 2H), 3.05 (s, 3H), 2.83-2.67 (m, 4H), 2.34-2.31 (m, 1H), 2.29-2.20 (m, 1H), 1.96-1.79 (m, 2H), 1.32 (s, 3H), 1.10 (s, 3H). LCMS (Method Q): 489.5 [M+H]⁺.

### Example 254: N-((1S)-1-(5-((4-Cyclopropyl-2,3-dihydro-1H-inden-2-yl)amino)pyridin-2-yl)-2,2,2-trifluoroethyl)-N-methyltetrahydro-2H-thiopyran-4-carboxamide 1,1-dioxide

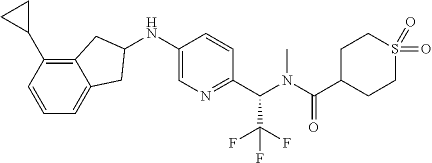 [SMILES: CN(C(=O)C1CCS(=O)(=O)CC1)[C@@H](c1ccc(NC2Cc3cccc(C4CC4)c3C2)cn1)C(F)(F)F]

The title compound (14 mg) was prepared in an analogous manner to Intermediate 11 from Intermediate 90 (0.25 g, 0.58 mmol), Intermediate 143 (0.12 g, 0.58 mmol), Cs₂CO₃ (0.48 g, 0.58 mmol), and RuPhosPdG3 (48 mg, 0.58 mmol) in toluene (5 mL) at 110° C. for 16 h. Purified by preparative HPLC (XBridge C18, 19×250 mm×5 μm, flow rate: 18 mL/min, 10 mM aqueous NH₄HCO₃ with MeCN 55% to 62% over 10 min, held for 9 min, ramped to 100% over 0.5 min and held for 7 min). Chiral purification was carried out by preparative SFC (Lux-i-Amylose-3, 30×250 mm×5 μm, temp 30° C., Back Pressure 100 bar, flow rate: 100 g/min, 50% CO₂ with 50% (NH₄HCO₃ in MeOH) modifier) to provide Example 254 as Peak 2. ¹H NMR (400 MHz; DMSO-de) δ: 8.07-8.03 (m, 1H), 7.27-7.00 (m, 4H), 6.68 (d, 1H), 6.54-6.48 (m, 1H), 6.41-6.01 (m, 1H), 4.29-4.25 (m, 1H), 3.43 (dd, 1H), 3.24-3.09 (m, 3H), 3.02 (s, 3H), 2.88-2.67 (m, 5H), 2.02-1.98 (m, 4H), 1.87-1.83 (m, 1H), 0.92-0.89 (m, 2H), 0.64-0.62 (m, 2H). LCMS (Method Q): 522.4 [M+H]⁺.

### Example 255: N-Methyl-N-((1S)-2,2,2-trifluoro-1-(5-((5-(trifluoromethyl)-2,3-dihydro-1H-inden-2-yl)amino)pyridin-2-yl)ethyl)tetrahydro-2H-thiopyran-4-carboxamide 1,1-dioxide—Isomer 1

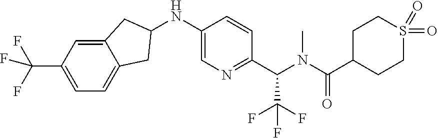 [SMILES: CN(C(=O)C1CCS(=O)(=O)CC1)[C@@H](c1ccc(NC2Cc3ccc(C(F)(F)F)cc3C2)cn1)C(F)(F)F]

### Example 256: N-Methyl-N-((1S)-2,2,2-trifluoro-1-(5-((5-(trifluoromethyl)-2,3-dihydro-1H-inden-2-yl)amino)pyridin-2-yl)ethyl)tetrahydro-2H-thiopyran-4-carboxamide 1,1-dioxide—Isomer 2

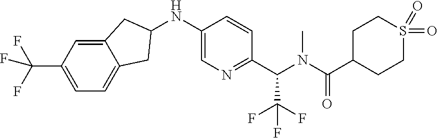 [SMILES: CN(C(=O)C1CCS(=O)(=O)CC1)[C@@H](c1ccc(NC2Cc3ccc(C(F)(F)F)cc3C2)cn1)C(F)(F)F]

The title compound was prepared in an analogous manner to Example 100 from tetrahydro-2H-thiopyran-4-carboxylic acid 1,1-dioxide (0.67 g, 3.74 mmol, CAS 64096-87-3), oxalyl chloride (0.53 mL, 6.23 mmol) in DCM (10 mL) at 0° C. then RT for 1 h, followed by addition of Intermediate 145 (0.50 g, 1.25 mmol), triethylamine (0.87 mL, 6.23 mmol) in DCM (10 mL) at RT for 2 h. Purified by preparative HPLC (X-Select C18, 19×250 mm×5 μm, flow rate: 18 mL/min, 10 mM aqueous NH₄HCO₃ with MeCN 50% to 60% over 11 min, held for 2 min, ramped to 100% over 0.5 min, held for 2 min). Chiral purification was carried out by preparative SFC (Chiralpak®-AD-H, 30×250 mm×5 μm, temp 30° C., Back Pressure 100 bar, flow rate: 100 g/min, 70% CO₂ with 30% MeOH modifier) to provide Example 255 (65 mg) as Peak 1 and Example 256 (73 mg) as Peak 2. Example 255: ¹H NMR (400 MHz; DMSO-d₆) δ: 8.05-8.01 (m, 1H), 7.61 (s, 1H), 7.53-7.45 (m, 2H), 7.27-7.13 (m, 1H), 7.08-7.01 (m, 1H), 6.56-6.50 (m, 1H), 6.41-6.03 (m, 1H), 4.34-4.31 (m, 1H), 3.39 (dd, 2H), 3.29-3.08 (m, 5H), 3.01 (s, 3H), 2.99 (dd, 2H), 2.08-1.97 (m, 4H). LCMS (Method A): 550.4 [M+H]⁺. Example 256: ¹H NMR (400 MHz; DMSO-d₆) δ: 8.05-8.01 (m, 1H), 7.61 (s, 1H), 7.53-7.45 (m, 2H), 7.27-7.13 (m, 1H), 7.08-7.01 (m, 1H), 6.56-6.50 (m, 1H), 6.41-6.03 (m, 1H), 4.34-4.30 (m, 1H), 3.37 (dd, 2H), 3.24-3.09 (m, 5H), 3.01 (s, 3H), 2.87 (dd, 2H), 2.16-1.95 (m, 4H). LCMS (Method A): 550.4 [M+H]⁺.

### Example 257: N-((1S)-1-(5-((4-Cyano-2,3-dihydro-1H-inden-2-yl)amino)pyridin-2-yl)-2,2,2-trifluoroethyl)-N-methyltetrahydro-2H-thiopyran-4-carboxamide 1,1-dioxide

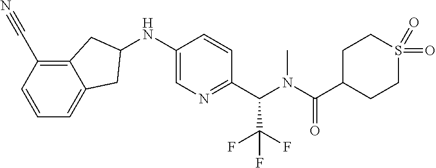 [SMILES: CN(C(=O)C1CCS(=O)(=O)CC1)[C@@H](c1ccc(NC2Cc3cccc(C#N)c3C2)cn1)C(F)(F)F]

The title compound (26 mg) was prepared in an analogous manner to Intermediate 11 from Intermediate 90 (0.40 g, 0.92 mmol), Intermediate 146 (0.26 g, 1.11 mmol), Cs₂CO₃ (0.90 g, 2.77 mmol), and RuPhosPdG3 (77 mg, 0.09 mmol) in toluene (8 mL) at 110° C. for 16 h. Purified by preparative HPLC (Gemini-NX C18, 19×250 mm×5 μm, flow rate: 18 mL/min, 10 mM aqueous NH₄HCO₃ with MeCN 40% to 60% over 7 min, held for 3.5 min, ramped to 100% over 0.1 min and held for 3.9 min). Chiral purification was carried out by preparative SFC (Lux-i-Amylose-3, 30×250 mm×5 μm, temp 30° C., Back Pressure 100 bar, flow rate: 100 g/min, 50% CO₂ with 50% MeOH modifier) to provide Example 257 as Peak 2. ¹H NMR (400 MHz; DMSO-d₆) δ: 8.05-8.01 (m, 1H), 7.64-7.58 (m, 2H), 7.37 (dd, 1H), 7.28-7.14 (m, 1H), 7.10-7.02 (m, 1H), 6.60-6.547 (m, 1H), 6.42-6.04 (m, 1H), 4.36-4.34 (m, 1H), 3.52 (dd, 1H), 3.42-3.36 (m, 2H), 3.25-3.09 (m, 4H), 3.01 (s, 3H), 2.99-2.89 (m, 2H), 2.17-2.07 (m, 4H). LCMS (Method A): 507.3 [M+H]⁺.

### Example 258: N-Methyl-N-((1S)-2,2,2-trifluoro-1-(5-((5-fluoro-2,3-dihydro-1H-inden-2-yl)amino)pyridin-2-yl)ethyl)tetrahydro-2H-thiopyran-4-carboxamide 1,1-dioxide—Isomer 1

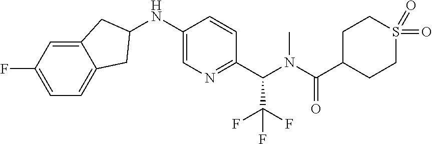 [SMILES: CN(C(=O)C1CCS(=O)(=O)CC1)[C@@H](c1ccc(NC2Cc3ccc(F)cc3C2)cn1)C(F)(F)F]

### Example 259: N-Methyl-N-((1S)-2,2,2-trifluoro-1-(5-((5-fluoro-2,3-dihydro-1H-inden-2-yl)amino)pyridin-2-yl)ethyl)tetrahydro-2H-thiopyran-4-carboxamide 1,1-dioxide—Isomer 2

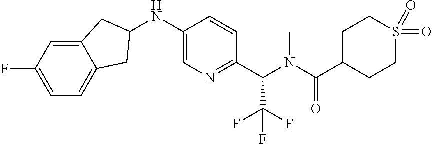 [SMILES: CN(C(=O)C1CCS(=O)(=O)CC1)[C@@H](c1ccc(NC2Cc3ccc(F)cc3C2)cn1)C(F)(F)F]

The title compound was prepared in an analogous manner to Intermediate 11 from Intermediate 90 (0.50 g, 0.86 mmol), 5-fluoro-2,3-dihydro-1H-inden-2-amine hydrochloride (0.23 g, 1.22 mmol, CAS 2340-07-0), Cs₂CO₃ (0.79 g, 2.45 mmol), and RuPhosPdG3 (68 mg, 0.08 mmol) in toluene (5 mL) at 110° C. for 16 h. Purified by preparative HPLC (YPC Triart exrs C18, 20×250 mm×5 μm, flow rate: 18 mL/min, 10 mM aqueous NH₄HCO₃ with MeCN 40% to 65% over 9 min, held for 4 min, ramped to 100% over 0.1 min and held for 4.9 min). Chiral purification was carried out by preparative SFC (Chiralpak® IC, 30×250 mm×5 μm, temp 30° C., Back Pressure 100 bar, flow rate: 100 g/min, 65% CO₂ with 35% MeOH modifier) to provide Example 258 (49 mg) as Peak 1 and Example 259 (59 mg) as Peak 2. Example 258: ¹H NMR (400 MHz; DMSO-d₆) δ: 8.04-8.00 (m, 1H), 7.24 (dd, 1H), 7.26 (d, 1H), 7.23-6.94 (m, 3H), 6.54-6.47 (m, 1H), 6.41-6.03 (m, 1H), 4.31-4.24 (m 1H), 3.27-3.08 (m, 7H), 3.01 (s, 3H), 2.83-2.73 (m, 2H), 2.07-1.98 (m, 4H). LCMS (Method R): 500.4 [M+H]⁺. Example 259: ¹H NMR (400 MHz; DMSO-d₆) δ: 8.04-8.01 (m, 1H), 7.24 (dd, 1H), 7.19 (d, 1H), 7.09-6.94 (m, 3H), 6.54-6.48 (m, 1H), 6.41-6.01 (m, 1H), 4.31-4.26 (m, 1H), 3.27-3.08 (m, 7H), 3.01 (s, 3H), 2.83-2.67 (m, 2H), 2.19-1.94 (m, 4H). LCMS (Method R): 500.4 [M+H]⁺.

### Example 260: (S)—N-(1-(5-((5,6-Dimethyl-2,3-dihydro-1H-inden-2-yl)amino)pyridin-2-yl)-2,2,2-trifluoroethyl)-N-methyltetrahydro-2H-thiopyran-4-carboxamide 1,1-dioxide

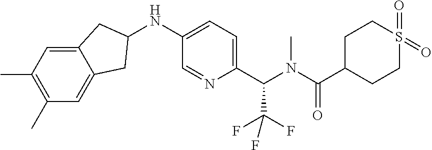 [SMILES: Cc1cc2c(cc1C)CC(Nc1ccc([C@H](N(C)C(=O)C3CCS(=O)(=O)CC3)C(F)(F)F)nc1)C2]

The title compound (20 mg) was prepared in an analogous manner to Intermediate 3 from Intermediate 148 (47 mg, 0.13 mmol), pyridine (0.7 mL), T3P® (50% MeTHF, 0.24 mL, 0.40 mmol) and tetrahydro-2H-thiopyran-4-carboxylic acid 1,1-dioxide (26 mg, 0.14 mmol, CAS 64096-87-3) at RT for 16 h. Purified by preparative HPLC (Gemini NX-C18, 21×250 mm×5 μm, flow rate: 30 mL/min, 0.1% aqueous formic acid with MeCN 50% to 57% over 6 min, held for 6 min, ramped to 95% over 0.2 min and held for 1.8 min). ¹H NMR (400 MHz; 353 K, DMSO-d₆) δ: 8.04 (d, 1H), 7.14 (br s, 1H), 7.07-7.00 (m, 3H), 6.40-6.30 (m, 1H), 6.16 (d, 1H), 4.25 (dd, 2H), 3.27 (dd, 2H), 3.25-3.10 (m, 4H), 2.76 (dd, 2H), 2.20 (s, 6H), 2.14-2.03 (m, 4H)—3H obscured by water signal. LCMS (Method M): 510.2 [M+H]⁺.

### Example 261: N-((1S)-1-(5-((4,5-Difluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

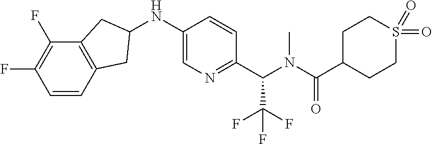 [SMILES: CN(C(=O)C1CCS(=O)(=O)CC1)[C@@H](c1ccc(NC2Cc3ccc(F)c(F)c3C2)cn1)C(F)(F)F]

### Example 262: N-((1S)-1-(5-((4,5-Difluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

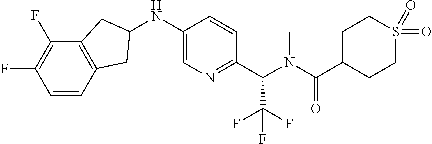 [SMILES: CN(C(=O)C1CCS(=O)(=O)CC1)[C@@H](c1ccc(NC2Cc3ccc(F)c(F)c3C2)cn1)C(F)(F)F]

The title compound was prepared in an analogous manner to Intermediate 11 from Intermediate 90 (0.50 g, 0.86 mmol), 4,5-difluoro-2,3-dihydro-1H-inden-2-amine hydrochloride (0.30 g, 1.29 mmol, CAS 180914-89-0), Cs₂CO₃ (0.84 g, 2.58 mmol), and RuPhosPdG3 (72 mg, 0.09 mmol) in toluene (10 mL) at 100° C. for 16 h. Purified by preparative HPLC (XBridge Phenyl, 19×250 mm×5 μm, flow rate: 17 mL/min, 10 mM aqueous NH₄HCO₃ with MeCN 30% to 65% over 14 min, held for 4 min, ramped to 100% over 0.1 min and held for 2.9 min). Chiral purification was carried out by preparative SFC (Chiralpak® AD-H, 30×250 mm×5 μm, temp 30° C., Back Pressure 100 bar, flow rate: 100 g/min, 60% CO₂ with 40% MeOH modifier) to provide Example 261 (13 mg) as Peak 1 and Example 262 (12 mg) as Peak 2. Example 261: ¹H NMR (400 MHz; DMSO-d₆) δ: 8.05-8.01 (m, 1H), 7.27-7.02 (m, 4H), 6.60-6.53 (m, 1H), 6.41-6.03 (m, 1H), 4.38-4.33 (m, 1H), 3.42 (dd, 2H), 3.22-3.09 (m, 5H), 3.01 (s, 3H), 2.88-2.67 (m, 2H), 2.16-1.94 (m, 4H). LCMS (Method A): 518.3 [M+H]⁺. Example 262: ¹H NMR (400 MHz; DMSO-d₆) δ: 8.05-8.01 (m, 1H), 7.27-7.01 (m, 4H), 6.60-6.53 (m, 1H), 6.41-6.06 (m, 1H), 4.37-4.34 (m, 1H), 3.41 (dd, 2H), 3.25-3.08 (m, 5H), 3.01 (s, 3H), 2.88-2.97 (m, 2H), 2.16-1.90 (m, 4H). LCMS (Method A): 518.3 [M+H]⁺.

### Example 263: N-((1S)-1-(5-((4-Chloro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

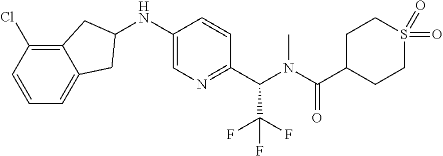 [SMILES: CN(C(=O)C1CCS(=O)(=O)CC1)[C@@H](c1ccc(NC2Cc3cccc(Cl)c3C2)cn1)C(F)(F)F]

### Example 264: N-((1S)-1-(5-((4-Chloro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

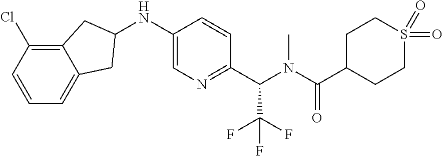 [SMILES: CN(C(=O)C1CCS(=O)(=O)CC1)[C@@H](c1ccc(NC2Cc3cccc(Cl)c3C2)cn1)C(F)(F)F]

The title compound was prepared in an analogous manner to Intermediate 3 from Intermediate 150 (0.50 g, 1.21 mmol), pyridine (0.5 mL), tetrahydro-2H-thiopyran-4-carboxylic acid 1,1-dioxide (0.65 g, 3.63 mmol, CAS 64096-87-3), T3P® (50% in EtOAc, 7.19 mL, 24.2 mmol) at RT for 16 h. Purified by preparative HPLC (YMC Triart C18, 19×250 mm×5 μm, flow rate: 18 mL/min, 10 mM aqueous NH₄HCO₃ with MeCN 40% to 65% over 14 min, held for 4.5 min, ramped to 100% over 0.1 min and held for 4.4 min). Chiral purification was carried out by preparative SFC (Chiralpak®-IC, 30×250 mm×5 μm, temp 30° C., Back Pressure 100 bar, flow rate: 100 g/min, 60% CO₂ with 40% MeOH modifier) to provide Example 263 (0.10 g) as Peak 1, Example 264 (0.10 g) as Peak 2. Example 263: ¹H NMR (400 MHz; DMSO-d₆) δ: 8.05-8.02 (m, 1H), 7.27-7.02 (m, 5H), 6.58-6.52 (m, 1H), 6.41-6.04 (m, 1H), 4.31-4.95 (m, 1H), 3.44-3.35 (m, 3H), 3.25-3.12 (m, 4H), 3.09 (s, 3H), 2.93-2.68 (m, 2H), 2.28-1.99 (m, 4H). LCMS (Method A): 516.3 [M+H]⁺. Example 264: ¹H NMR (400 MHz; DMSO-d₆) δ: 8.05-8.02 (m, 1H), 7.28-7.02 (m, 5H), 6.58-6.52 (m, 1H), 6.41-6.04 (m, 1H), 4.32-4.28 (m, 1H), 3.57-3.35 (m, 3H), 3.32-3.12 (m, 4H), 3.01 (s, 3H), 2.93-2.68 (m, 2H), 2.28-2.01 (m, 4H). LCMS (Method A): 516.3 [M+H]⁺.

### Example 265: N-((1S)-1-(5-((4-Chloro-5-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

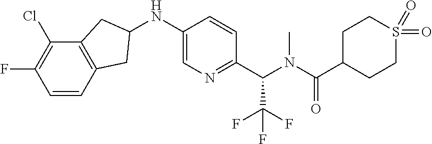 [SMILES: CN(C(=O)C1CCS(=O)(=O)CC1)[C@@H](c1ccc(NC2Cc3ccc(F)c(Cl)c3C2)cn1)C(F)(F)F]

### Example 266: N-((1S)-1-(5-((4-Chloro-5-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

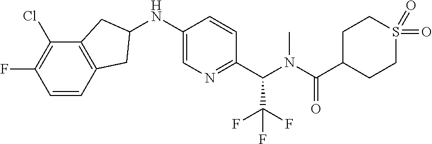 [SMILES: CN(C(=O)C1CCS(=O)(=O)CC1)[C@@H](c1ccc(NC2Cc3ccc(F)c(Cl)c3C2)cn1)C(F)(F)F]

To a stirred solution of Intermediate 166 (0.25 g, 0.66 mmol) and Intermediate 153 (0.72 g, 1.33 mmol) in MeOH (2.5 mL) was added AcOH (0.04 mL, 0.73 mmol) and the mixture was stirred at 60° C. for 8 h. Sodium cyanoborohydride (83 mg, 1.33 mmol) was added and the mixture stirred at RT for 16 h. The mixture was poured into water and extracted with EtOAc. The organics were washed with water, brine, dried over Na₂SO₄, filtered and concentrated under reduced pressure. The crude product was purified by preparative HPLC (XBridge Phenyl, 19×250 mm×5 μm, flow rate: 18 mL/min, 10 mM aqueous NH₄HCO₃ with MeCN 40% to 70% over 14 min, held for 1 min, ramped to 100% over 0.1 min and held for 2.9 min). Chiral purification was carried out by preparative SFC (Chiralpak®-AD-H, 30×250 mm×5 μm, temp 30° C., Back Pressure 100 bar, flow rate: 100 g/min, 60% CO₂ with 40% MeOH modifier) to provide Example 265 (5 mg) as Peak 1, Example 266 (7 mg) as Peak 2. Example 265: ¹H NMR (400 MHz; DMSO-d₆) δ: 8.04-8.01 (m, 1H), 7.27-7.02 (m, 4H), 6.59-6.53 (m, 1H), 6.41-6.03 (m, 1H), 4.35-4.20 (m, 1H), 3.46-3.35 (m, 2H), 3.30-3.08 (m, 5H), 3.02 (s, 3H), 2.82-2.67 (m, 2H), 2.20-1.93 (m, 4H). LCMS (Method S): 534.2 [M+H]⁺. Example 266: ¹H NMR (400 MHz; DMSO-d₆) δ: 8.04-8.01 (m, 1H), 7.27-7.02 (m, 4H), 6.59-6.53 (m, 1H), 6.41-6.03 (m, 1H), 4.34 (dd, 1H), 3.44-3.38 (m, 2H), 3.30-3.11 (m, 5H), 3.01 (s, 3H), 2.91-2.80 (m, 2H), 2.20-1.94 (m, 4H). LCMS (Method S): 534.2 [M+H]⁺.

### Example 267: (S)—N-(1-(5-((4,7-Dimethyl-2,3-dihydro-1H-inden-2-yl)amino)pyridin-2-yl)-2,2,2-trifluoroethyl)-N-methyltetrahydro-2H-thiopyran-4-carboxamide 1,1-dioxide

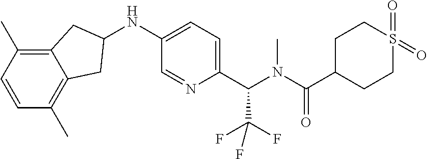 [SMILES: Cc1ccc(C)c2c1CC(Nc1ccc([C@H](N(C)C(=O)C3CCS(=O)(=O)CC3)C(F)(F)F)nc1)C2]

The title compound (8 mg) was prepared in an analogous manner to Intermediate 3 from Intermediate 156 (29 mg, 0.08 mmol), pyridine (0.4 mL), T3P® (50% MeTHF, 0.15 mL, 0.25 mmol) and tetrahydro-2H-thiopyran-4-carboxylic acid 1,1-dioxide (16 mg, 0.09 mmol, CAS 64096-87-3) at RT for 16 h. Purified by preparative HPLC (Gemini NX-C18, 21×250 mm×5 μm, flow rate: 30 mL/min, 0.1% aqueous formic acid with MeCN 50% to 60% over 6 min, held for 5 min, ramped to 95% over 0.2 min and held for 1.8 min). ¹H NMR (400 MHz; 353 K, DMSO-d₆) δ: 8.06 (d, 1H), 7.19-7.14 (m, 1H), 7.06 (d, 1H), 6.89 (s, 2H), 6.39 (br s, 1H), 6.21 (d, 1H), 4.33-4.26 (m, 1H), 3.31 (dd, 2H), 3.25-2.20 (m, 5H), 2.76 (dd, 2H), 2.18 (s, 6H), 2.14-2.04 (m, 4H)—3H obscured by water signal. LCMS (Method M): 510.3 [M+H]⁺.

### Example 268: N-((1S)-1-(5-((4,6-Difluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

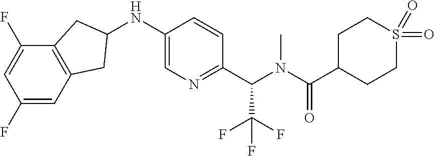 [SMILES: CN(C(=O)C1CCS(=O)(=O)CC1)[C@@H](c1ccc(NC2Cc3cc(F)cc(F)c3C2)cn1)C(F)(F)F]

### Example 269: N-((1S)-1-(5-((4,6-Difluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

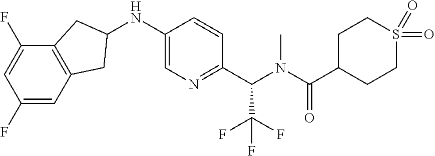 [SMILES: CN(C(=O)C1CCS(=O)(=O)CC1)[C@@H](c1ccc(NC2Cc3cc(F)cc(F)c3C2)cn1)C(F)(F)F]

The title compound was prepared in an analogous manner to Intermediate 11 from Intermediate 90 (0.45 g, 0.86 mmol), 4,6-difluoro-2,3-dihydro-1H-inden-2-amine hydrochloride (0.21 g, 0.95 mmol, CAS 1008361-87-2), Cs₂CO₃ (0.84 g, 2.58 mmol), and RuPhosPdG3 (72 mg, 0.09 mmol) in toluene (4.5 mL) at 110° C. for 16 h. Purified by preparative HPLC (X-Select C18, 19×250 mm×5 μm, flow rate: 17 mL/min, 10 mM aqueous NH₄HCO₃ with MeCN 30% to 60% over 7 min, held for 6.4 min, ramped to 100% over 0.1 min and held for 8.5 min). Chiral purification was carried out by preparative SFC (Chiralpcel IC, 30×250 mm×5 μm, temp 30° C., Back Pressure 100 bar, flow rate: 100 g/min, 73% CO₂ with 27% MeOH modifier) to provide Example 268 (40 mg) as Peak 1 and Example 269 (38 mg) as Peak 2. Example 268: ¹H NMR (400 MHz; DMSO-d₆) δ: 8.05-8.01 (m, 1H), 7.27-7.13 (m, 1H), 7.08-6.98 (m, 3H), 6.58-6.52 (m, 1H), 6.41-6.01 (m, 1H), 4.35-4.30 (m, 1H), 3.38-3.36 (m, 1H), 3.30-3.08 (m, 6H), 3.01 (s, 3H), 2.88-2.67 (m, 2H), 2.19-1.94 (m, 4H). LCMS (Method S): 518.2 [M+H]⁺. Example 269: ¹H NMR (400 MHz; DMSO-d₆) δ: 8.05-8.01 (m, 1H), 7.27-7.13 (m, 1H), 7.08-6.98 (m, 3H), 6.58-6.52 (m, 1H), 6.41-6.02 (m, 1H), 4.36-4.32 (m, 1H), 3.39-3.36 (m, 1H), 3.30-3.08 (m, 6H), 3.01 (s, 3H), 2.88-2.74 (m, 2H), 2.16-1.94 (m, 4H). LCMS (Method S): 518.3 [M+H]⁺.

### Example 270: N-((1S)-1-(5-((6-Chloro-4-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

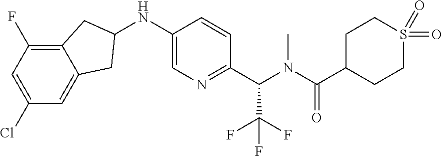 [SMILES: CN(C(=O)C1CCS(=O)(=O)CC1)[C@@H](c1ccc(NC2Cc3cc(Cl)cc(F)c3C2)cn1)C(F)(F)F]

### Example 271: N-((1S)-1-(5-((6-Chloro-4-fluoro-2,3-dihydro-1H-inden-2-ylamino)pyridin-2-yl)-2,2,2-trifluoroethyl)-N-methyltetrahydro-2H-thiopyran-4-carboxamide 1,1-dioxide—Isomer 2

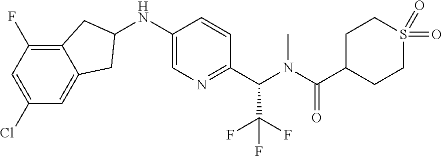 [SMILES: CN(C(=O)C1CCS(=O)(=O)CC1)[C@@H](c1ccc(NC2Cc3cc(Cl)cc(F)c3C2)cn1)C(F)(F)F]

The title compound was prepared in an analogous manner to Intermediate 3 from Intermediate 158 (0.19 g, 0.44 mmol), pyridine (0.6 mL), tetrahydro-2H-thiopyran-4-carboxylic acid 1,1-dioxide (0.16 g, 0.88 mmol, CAS 64096-87-3), T3P® (50% in MeTHF, 1.41 mL, 4.42 mmol) at RT for 16 h. Purified by preparative HPLC (YMC Triart C18, 20×250 mm×5 μm, flow rate: 17 mL/min, 10 mM aqueous NH₄HCO₃ with MeCN 40% to 75% over 14 min, held for 3 min, ramped to 100% over 0.1 min and held for 1.9 min). Chiral purification was carried out by preparative SFC (Lux-i-Amylose-3, 30×250 mm×5 μm, temp 30° C., Back Pressure 100 bar, flow rate: 100 g/min, 50% CO₂ with 50% (MeOH/MeCN 1:1) modifier) to provide Example 270 (31 mg) as Peak 1, Example 271 (51 mg) as Peak 2. Example 270: ¹H NMR (400 MHz; DMSO-d₆) δ: 8.04-8.00 (m, 1H), 7.23-7.13 (m, 3H), 7.08-7.01 (m, 1H), 6.58-6.52 (m, 1H), 6.41-6.02 (m, 1H), 4.36-4.32 (m, 1H), 3.39-3.37 (m, 1H), 3.27-3.09 (m, 6H), 3.01 (s, 3H), 2.89-2.76 (m, 2H), 2.19-1.95 (m, 4H). LCMS (Method A): 534.3 [M+H]⁺. Example 271: ¹H NMR (400 MHz; DMSO-d₆) δ: 8.04-8.00 (m, 1H), 7.23-7.13 (m, 3H), 7.09-7.01 (m, 1H), 6.58-6.52 (m, 1H), 6.40-6.02 (m, 1H), 4.36-4.32 (m, 1H), 3.38-3.32 (m, 1H), 3.18-3.12 (m, 6H), 3.01 (s, 3H), 2.89-2.76 (m, 2H), 2.16-1.94 (m, 4H). LCMS (Method A): 534.3 [M+H]⁺.

### Example 272: (S)—N-(1-(5-((5,6-Difluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide

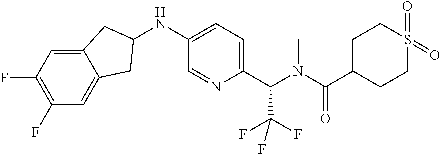 [SMILES: CN(C(=O)C1CCS(=O)(=O)CC1)[C@@H](c1ccc(NC2Cc3cc(F)c(F)cc3C2)cn1)C(F)(F)F]

The title compound (5 mg) was prepared in an analogous manner to Intermediate 3 from Intermediate 160 (10 mg, 0.03 mmol), pyridine (0.1 mL), T3P® (50% MeTHF, 0.05 mL, 0.08 mmol) and tetrahydro-2H-thiopyran-4-carboxylic acid 1,1-dioxide (5 mg, 0.03 mmol, CAS 64096-87-3) at RT for 16 h. Purified by preparative HPLC (Gemini NX-C18, 21×250 mm×5 μm, flow rate: 30 mL/min, 0.1% aqueous formic acid with MeCN 50% to 60% over 10 min, held for 1 min, ramped to 95% over 0.2 min and held for 1.8 min). ¹H NMR (400 MHz; DMSO-d₆) δ: 8.04-8.00 (m, 1H), 7.31 (dd, 2H), 7.14 (d, 1H), 7.06-7.00 (m, 1H), 5.62-5.56 (m, 1H), 6.38 (dd, 1H), 4.30 (dd, 1H), 3.30-3.10 (m, 7H), 3.01 (s, 3H), 2.78 (dd, 2H), 2.10-1.98 (m, 4H). LCMS (Method M): 518.2 [M+H]⁺.

### Example 273: (S)—N-(1-(5-((4,7-Difluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide

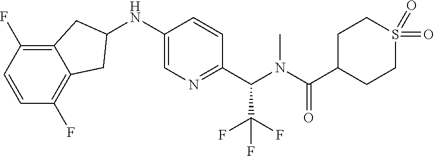 [SMILES: CN(C(=O)C1CCS(=O)(=O)CC1)[C@@H](c1ccc(NC2Cc3c(F)ccc(F)c3C2)cn1)C(F)(F)F]

The title compound (5 mg) was prepared in an analogous manner to Intermediate 11 from Intermediate 90 (36 mg, 0.09 mmol), 4,7-difluoro-2,3-dihydro-1H-inden-2-amine (15 mg, 0.09 mmol, CAS 173998-68-0), Cs₂CO₃ (82 mg, 0.25 mmol), and RuPhosPdG3 (11 mg, 0.01 mmol) in toluene (0.4 mL) at 110° C. for 14 h. Purified by preparative HPLC (Gemini-NX C18, 21×250 mm×5 μm, flow rate: 30 mL/min, 0.1% aqueous formic acid with MeCN 40% to 55% over 6 min, held for 6 min, ramped to 95% over 0.2 min and held for 1.8 min). ¹H NMR (400 MHz; 353 K, DMSO-d₆) δ: 8.58 (s, 1H), 8.06 (d, 1H), 7.19-7.15 (m, 1H), 7.07 (d, 1H), 7.02 (dd, 2H), 6.37-6.31 (m, 1H), 4.48-4.41 (m, 1H), 3.42 (dd, 2H), 3.26-3.13 (m, 5H), 2.89 (dd, 2H), 2.16-2.03 (m, 4H)—3H obscured by water signal. LCMS (Method M): 518.2 [M+H]⁺.

### Example 274: N-Methyl-N-((1S)-2,2,2-trifluoro-1-(5-((4-fluoro-2,3-dihydro-1H-inden-2-yl)amino)pyridin-2-yl)ethyl)tetrahydro-2H-thiopyran-4-carboxamide 1,1-dioxide—Isomer 1

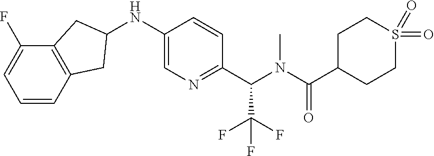 [SMILES: CN(C(=O)C1CCS(=O)(=O)CC1)[C@@H](c1ccc(NC2Cc3cccc(F)c3C2)cn1)C(F)(F)F]

### Example 275: N-Methyl-N-((1S)-2,2,2-trifluoro-1-(5-((4-fluoro-2,3-dihydro-1H-inden-2-yl)amino)pyridin-2-yl)ethyl)tetrahydro-2H-thiopyran-4-carboxamide 1,1-dioxide—Isomer 2

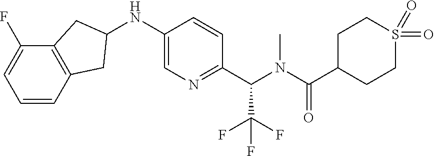 [SMILES: CN(C(=O)C1CCS(=O)(=O)CC1)[C@@H](c1ccc(NC2Cc3cccc(F)c3C2)cn1)C(F)(F)F]

The title compound was prepared in an analogous manner to Intermediate 11 from Intermediate 90 (0.45 g, 1.23 mmol), 4-fluoro-2,3-dihydro-1H-inden-2-amine hydrochloride (0.35 g, 1.35 mmol, CAS 2340-05-8), Cs₂CO₃ (1.21 g, 3.71 mmol), and RuPhosPdG3 (0.10 g, 0.12 mmol) in toluene (5 mL) at 110° C. for 16 h. Purified by preparative HPLC (XBridge Phenyl, 19×250 mm×5 μm, flow rate: 18 mL/min, 10 mM aqueous NH₄HCO₃ with MeCN 20% to 50% over 9 min, held for 3 min, ramped to 100% over 0.1 min and held for 3.9 min). Chiral purification was carried out by preparative SFC (Chiralpcel IC, 30×250 mm×5 μm, temp 30° C., Back Pressure 100 bar, flow rate: 100 g/min, 73% CO₂ with 27% MeOH modifier) to provide Example 274 (17 mg) as Peak 1 and Example 275 (14 mg) as Peak 2. Example 274: ¹H NMR (400 MHz; DMSO-d₆) δ: 8.05-8.01 (m, 1H), 7.21-6.96 (m, 5H), 6.58-6.54 (m, 1H), 6.52-6.01 (m, 1H), 4.35-4.28 (m, 1H), 3.39-3.20 (m, 2H), 3.24-3.08 (m, 5H), 3.01 (s, 3H), 2.88-2.78 (m, 2H), 2.19-1.95 (m, 4H). LCMS (Method A): 500.4 [M+H]⁺. Example 275: ¹H NMR (400 MHz; DMSO-d₆) δ: 8.05-8.01 (m, 1H), 7.29-6.96 (m, 5H), 6.56-6.52 (m, 1H), 6.41-6.03 (m, 1H), 4.35-4.28 (m, 1H), 3.39-3.27 (m, 2H), 3.25-3.08 (m, 5H), 3.01 (s, 3H), 2.88-2.78 (m, 2H), 2.19-1.95 (m, 4H). LCMS (Method A): 500.4 [M+H]⁺.

### Example 276: N-((1S)-1-(5-((5-Chloro-4-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

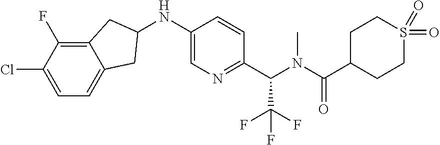 [SMILES: CN(C(=O)C1CCS(=O)(=O)CC1)[C@@H](c1ccc(NC2Cc3ccc(Cl)c(F)c3C2)cn1)C(F)(F)F]

### Example 277: N-((1S)-1-(5-((5-Chloro-4-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

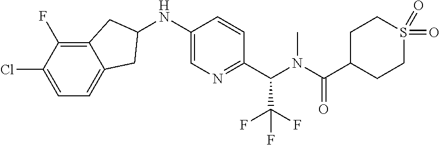 [SMILES: CN(C(=O)C1CCS(=O)(=O)CC1)[C@@H](c1ccc(NC2Cc3ccc(Cl)c(F)c3C2)cn1)C(F)(F)F]

The title compound was prepared in an analogous manner to Intermediate 3 from Intermediate 162 (0.22 g, 0.45 mmol), pyridine (0.7 mL), tetrahydro-2H-thiopyran-4-carboxylic acid 1,1-dioxide (0.24 g, 1.36 mmol, CAS 64096-87-3), T3P® (50% in MeTHF, 1.44 mL, 4.53 mmol) at RT for 16 h. Purified by preparative HPLC (XBridge C18, 19×250 mm×5 μm, flow rate: 18 mL/min, 10 mM aqueous NH₄HCO₃ with MeCN 30% to 68% over 10 min, held for 4 min, ramped to 100% over 0.1 min and held for 5.9 min). Chiral purification was carried out by preparative SFC (Chiralpak®-IC, 30×250 mm×5 μm, temp 30° C., Back Pressure 100 bar, flow rate: 100 g/min, 63% CO₂ with 37% MeOH modifier) to provide Example 276 (24 mg) as Peak 1, Example 277 (24 mg) as Peak 2. Example 276: ¹H NMR (400 MHz; DMSO-d₆) δ: 8.05-8.01 (m, 1H), 7.37 (dd, 1H), 7.28-7.01 (m, 3H), 6.59-6.01 (m, 2H), 4.38-4.32 (m, 1H), 3.44-3.37 (m, 2H), 3.32-3.12 (m, 5H), 3.01 (s, 3H), 2.85 (dd, 2H), 2.18-1.95 (m, 4H). LCMS (Method A): 534.3 [M+H]⁺. Example 277: ¹H NMR (400 MHz; DMSO-d₆) δ: 8.05-8.01 (m, 1H), 7.37 (dd, 1H), 7.27-7.01 (m, 3H), 6.59-6.53 (m, 1H), 6.41-6.40 (m, 1H), 4.37-4.33 (m, 1H), 3.44-3.32 (m, 2H), 3.24-3.09 (m, 5H), 3.01 (s, 3H), 2.87-2.67 (m, 2H), 2.18-1.94 (m, 4H). LCMS (Method A): 534.3 [M+H]⁺.

### Example 278: N-((1S)-1-(5-((4,5-Dichloro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

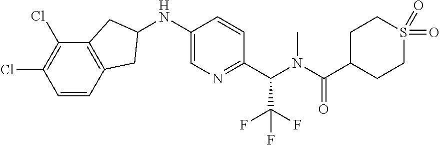 [SMILES: CN(C(=O)C1CCS(=O)(=O)CC1)[C@@H](c1ccc(NC2Cc3ccc(Cl)c(Cl)c3C2)cn1)C(F)(F)F]

### Example 279: N-((1S)-1-(5-((4,5-Dichloro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

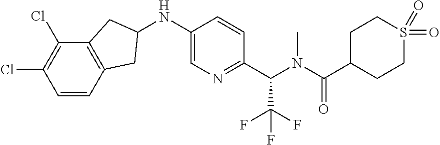 [SMILES: CN(C(=O)C1CCS(=O)(=O)CC1)[C@@H](c1ccc(NC2Cc3ccc(Cl)c(Cl)c3C2)cn1)C(F)(F)F]

The title compound was prepared in an analogous manner to Example 265 from Intermediate 166 (0.25 g, 0.66 mmol), 4,5-dichloro-1,3-dihydro-2H-inden-2-one (0.72 g, 1.33 mmol, CAS 69392-70-7), AcOH (0.04 mL, 0.66 mmol) in MeOH (2.5 mL) at 60° C. for 8 h then sodium cyanoborohydride (83 mg, 1.33 mmol) at RT for 16 h. Purified by flash chromatography (silica gel, eluting 0-80% EtOAc in hexane). Chiral purification was carried out by preparative SFC (Lux-i-Amylose-3, 30×250 mm×5 μm, temp 30° C., Back Pressure 100 bar, flow rate: 100 g/min, 55% CO₂ with 45% (MeOH/MeCN 1:1) modifier) to provide Example 278 (25 mg) as Peak 1, Example 279 (33 mg) as Peak 2. Example 278: ¹H NMR (400 MHz; DMSO-d₆) δ: 8.05-8.00 (m, 1H), 7.44 (d, 1H), 7.28-7.24 (m, 1H), 7.15 (d, 1H), 7.08-7.01 (m, 1H), 6.59-6.54 (m, 1H), 6.41-6.04 (m, 1H), 4.34-4.32 (m, 1H), 3.46-3.36 (m, 2H), 3.20-3.08 (m, 5H), 3.01 (s, 3H), 2.92-2.84 (m, 2H), 2.21-1.94 (m, 4H). LCMS (Method A): 550.3 [M+H]⁺. Example 279: ¹H NMR (400 MHz; DMSO-d₆) δ: 8.05-8.00 (m, 1H), 7.44 (d, 1H), 7.28-7.24 (m, 1H), 7.15 (d, 1H), 7.08-7.01 (m, 1H), 6.59-6.54 (m, 1H), 6.41-6.2 (m, 1H), 4.35-4.32 (m, 1H), 3.46-3.36 (m, 2H), 3.25-3.08 (m, 5H), 3.01 (s, 3H), 2.92-2.85 (m, 2H), 2.18-1.94 (m, 4H). LCMS (Method A): 550.3 [M+H]⁺.

### Example 280: N-Methyl-N-((1S)-2,2,2-trifluoro-1-(6-((4-(trifluoromethyl)-2,3-dihydro-1H-inden-2-yl)amino)pyridin-3-yl)ethyl)tetrahydro-2H-thiopyran-4-carboxamide 1,1-dioxide—Isomer 1

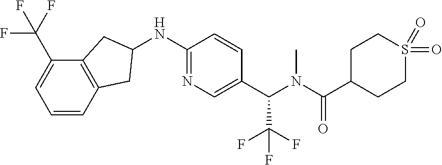 [SMILES: CN(C(=O)C1CCS(=O)(=O)CC1)[C@@H](c1ccc(NC2Cc3cccc(C(F)(F)F)c3C2)nc1)C(F)(F)F]

### Example 281: N-Methyl-N-((1S)-2,2,2-trifluoro-1-(6-((4-(trifluoromethyl)-2,3-dihydro-1H-inden-2-yl)amino)pyridin-3-yl)ethyl)tetrahydro-2H-thiopyran-4-carboxamide 1,1-dioxide—Isomer 2

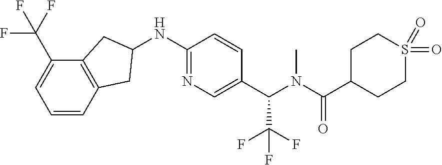 [SMILES: CN(C(=O)C1CCS(=O)(=O)CC1)[C@@H](c1ccc(NC2Cc3cccc(C(F)(F)F)c3C2)nc1)C(F)(F)F]

The title compound was prepared in an analogous manner to Intermediate 3 from Intermediate 167 (0.50 g, 1.23 mmol), pyridine (1.5 mL), tetrahydro-2H-thiopyran-4-carboxylic acid 1,1-dioxide (1.10 g, 6.16 mmol, CAS 64096-87-3), T3P® (50% in MeTHF, 7.34 mL, 12.3 mmol) at RT for 16 h. Purified by preparative HPLC (XBridge C18, 19×250 mm×5 μm, flow rate: 18 mL/min, 10 mM aqueous NH₄HCO₃ with MeCN 30% to 75% over 14 min, held for 1.5 min, ramped to 100% over 0.1 min and held for 2.4 min). Chiral purification was carried out by preparative SFC (Chiralpak®-AD-H, 30×250 mm×5 μm, temp 30° C., Back Pressure 100 bar, flow rate: 100 g/min, 63% CO₂ with 37% MeOH modifier) to provide Example 280 (58 mg) as Peak 1, Example 281 (54 mg) as Peak 2. Example 280: ¹H NMR (400 MHz; DMSO-d₆) δ: 8.12-8.00 (m, 1H), 7.56 (d, 1H), 7.51 (d, 1H), 7.47-7.35 (m, 2H), 7.29-7.22 (m, 1H), 6.60-6.56 (d, 1H), 6.41-6.23 (m, 1H), 4.67 (dd, 1H), 3.46 (dd, 1H), 3.38-3.36 (m 1H), 3.26-3.12 (m, 5H), 3.09-2.88 (m, 5H) 2.10-2.03 (m, 4H). LCMS (Method A): 550.4 [M+H]⁺. Example 281: ¹H NMR (400 MHz; DMSO-d₆) δ: 8.12-8.01 (s, 1H), 7.56 (d, 1H), 7.50 (d, 1H), 7.40-7.36 (m, 2H), 7.29-7.22 (m, 1H), 6.60-6.54 (m, 1H), 6.41-6.01 (m, 1H), 4.67 (dd, 1H), 3.45 (dd, 1H), 3.38-3.36 (m, 1H), 3.25-3.12 (m, 5H), 3.09-2.87 (m, 5H), 2.11-2.03 (m, 4H). LCMS (Method A): 550.4 [M+H]⁺.

### Example 282: N-((1S)-1-(5-((4,6-Dichloro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

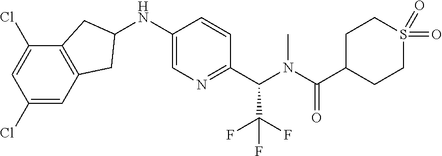 [SMILES: CN(C(=O)C1CCS(=O)(=O)CC1)[C@@H](c1ccc(NC2Cc3cc(Cl)cc(Cl)c3C2)cn1)C(F)(F)F]

### Example 283: N-((1S)-1-(5-((4,6-Dichloro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

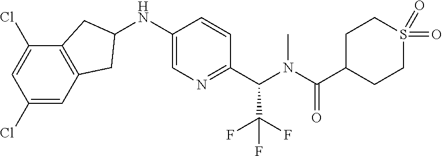 [SMILES: CN(C(=O)C1CCS(=O)(=O)CC1)[C@@H](c1ccc(NC2Cc3cc(Cl)cc(Cl)c3C2)cn1)C(F)(F)F]

The title compound was prepared in an analogous manner to Example 265 from Intermediate 166 (0.25 g, 0.63 mmol), 4,6-dichloro-1,3-dihydro-2H-inden-2-one (0.49 g, 0.94 mmol, CAS 1187983-76-1), AcOH (0.09 mL, 1.61 mmol) and MgSO₄ (76 mg, 0.63 mmol) in DCE (10 mL) at 60° C. for 4 h then sodium cyanoborohydride (0.12 g, 1.89 mmol) at RT for 16 h. Purified by flash chromatography (silica gel, eluting 0-80% EtOAc in hexane). Chiral purification was carried out by preparative SFC (Lux-Cellulose-4, 30×250 mm×5 μm, temp 30° C., Back Pressure 100 bar, flow rate: 100 g/min, 55% CO₂ with 45% (MeOH/MeCN 1:1) modifier) to provide Example 282 (26 mg) as Peak 1, Example 283 (25 mg) as Peak 2. Example 282: ¹H NMR (400 MHz; DMSO-d₆) δ: 8.04-8.00 (m, 1H), 7.39 (s, 1H), 7.34 (s, 1H), 7.28-7.13 (m, 1H), 7.08-7.01 (m, 1H), 6.59-6.52 (m, 1H), 6.41-6.34 (m, 1H), 4.33-3.31 (m, 1H), 3.44-3.37 (m, 2H), 3.17-3.11 (m, 5H), 3.09 (s, 3H), 2.93 (dd, 1H), 2.80 (dd, 1H), 2.16-1.94 (m, 4H). LCMS (Method A): 550.3 [M+H]⁺. Example 283: ¹H NMR (400 MHz; DMSO-d₆) δ: 8.04-8.00 (m, 1H), 7.39 (s, 1H), 7.34 (s, 1H), 7.28-7.13 (m, 1H), 7.08-7.00 (m, 1H), 6.59-6.53 (m, 1H), 6.41-6.04 (m, 1H) 4.35-4.31 (m, 1H), 3.44-3.37 (m, 2H), 3.16-3.12 (m, 5H), 3.01 (s, 3H), 2.91 (dd, 1H), 2.80 (dd, 1H), 2.16-1.94 (m, 4H). LCMS (Method A): 550.3 [M+H]⁺.

### Example 284: N-((1S)-1-(5-((5-Bromo-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

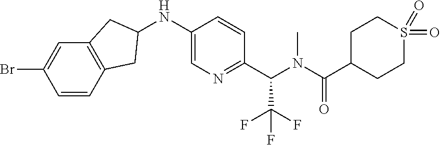 [SMILES: CN(C(=O)C1CCS(=O)(=O)CC1)[C@@H](c1ccc(NC2Cc3ccc(Br)cc3C2)cn1)C(F)(F)F]

### Example 285: N-((1S)-1-(5-((5-Bromo-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

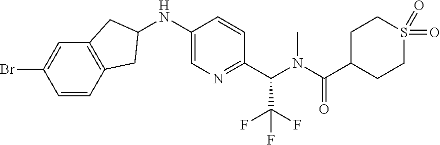 [SMILES: CN(C(=O)C1CCS(=O)(=O)CC1)[C@@H](c1ccc(NC2Cc3ccc(Br)cc3C2)cn1)C(F)(F)F]

The title compound was prepared in an analogous manner to Example 265 from Intermediate 166 (0.35 g, 0.88 mmol), 5-bromo-1,3-dihydro-2H-inden-2-one (0.37 g, 1.76 mmol, CAS 174349-93-0), AcOH (0.06 mL, 0.88 mmol) in MeOH (5 mL) at 60° C. for 4 h then sodium cyanoborohydride (0.11 g, 1.76 mmol) at RT for 16 h. Purified by preparative HPLC (XBridge C18, 30×150 mm×5 μm, flow rate: 18 mL/min, 10 mM aqueous NH₄HCO₃ with MeCN 20% to 65% over 9 min, held for 4 min, ramped to 100% over 0.1 min and held for 2.9 min). Chiral purification was carried out by preparative SFC (Lux-i-Amylose-3, 30×250 mm×5 μm, temp 30° C., Back Pressure 100 bar, flow rate: 100 g/min, 55% CO₂ with 45% (MeOH/MeCN 1:1) modifier) to provide Example 284 (49 mg) as Peak 1, Example 285 (55 mg) as Peak 2. Example 284: ¹H NMR (400 MHz; DMSO-d₆) δ: 8.04-8.00 (m, 1H), 7.45 (s, 1H), 7.29 (d, 1H), 7.24-7.14 (m, 1H), 7.12 (d, 1H), 7.04-7.00 (m, 1H), 6.53-6.47 (m, 1H), 6.41-6.03 (m, 1H), 4.31-4.24 (m, 1H), 3.56-3.09 (m, 7H), 3.01 (s, 3H), 2.84-2.67 (m, 2H), 2.16-1.94 (m, 4H). LCMS (Method A): 562.3 [M+H]⁺. Example 285: ¹H NMR (400 MHz; DMSO-d₆) δ: 8.04-8.00 (m, 1H), 7.45 (s, 1H) 7.34 (d, 1H), 7.24-7.20 (m, 1H), 7.14 (d, 1H), 7.13-7.00 (m, 1H), 6.53-6.47 (d, 1H), 6.41-6.03 (m, 1H), 4.29-4.24 (m, 1H), 3.24-3.09 (m, 7H), 3.01 (s, 3H), 2.83-2.73 (m, 2H), 2.16-1.95 (m, 4H). LCMS (Method A): 560.3 [M+H]⁺.

### Example 286: N-((1S)-1-(5-((4-Chloro-6-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

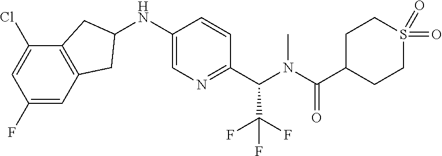 [SMILES: CN(C(=O)C1CCS(=O)(=O)CC1)[C@@H](c1ccc(NC2Cc3cc(F)cc(Cl)c3C2)cn1)C(F)(F)F]

### Example 287: N-((1S)-1-(5-((4-Chloro-6-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

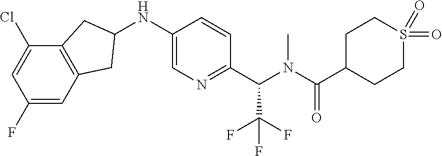 [SMILES: CN(C(=O)C1CCS(=O)(=O)CC1)[C@@H](c1ccc(NC2Cc3cc(F)cc(Cl)c3C2)cn1)C(F)(F)F]

The title compound was prepared in an analogous manner to Example 265 from Intermediate 166 (0.31 g, 0.82 mmol), Intermediate 168 (0.62 g, 1.88 mmol), AcOH (0.05 mL, 0.82 mmol) in MeOH (5 mL) at 60° C. for 4 h then sodium cyanoborohydride (0.10 g, 1.64 mmol) at RT for 16 h. Purified by preparative HPLC (XBridge Phenyl, 19×250 mm×5 μm, flow rate: 18 mL/min, 10 mM aqueous NH₄HCO₃ with MeCN 30% to 60% over 9 min, held for 7 min, ramped to 100% over 0.1 min and held for 0.9 min). Chiral purification was carried out by preparative SFC (Chiralpak®-IC, 30×250 mm×5 μm, temp 30° C., Back Pressure 100 bar, flow rate: 100 g/min, 55% CO₂ with 45% MeOH modifier) to provide Example 286 (32 mg) as Peak 1, Example 287 (28 mg) as Peak 2. Example 286: ¹H NMR (400 MHz; DMSO-d₆) δ: 8.05-8.01 (m, 1H), 7.28-7.01 (m, 4H), 6.59-6.53 (m, 1H), 6.41-6.02 (m, 1H), 4.35-4.30 (m, 1H), 3.44-3.32 (m, 2H), 3.24-3.09 (m, 5H), 3.01 (s, 3H), 2.91 (dd, 1H), 2.78 (dd, 1H), 2.16-1.90 (m, 4H). LCMS (Method A): 534.3 [M+H]1. Example 287: ¹H NMR (400 MHz; DMSO-d₆) δ: 8.05-8.01 (m, 1H), 7.28-7.01 (m, 4H), 6.59-6.53 (m, 1H), 6.41-6.02 (m, 1H), 4.34-4.31 (m, 1H), 3.44-3.38 (m, 2H), 3.32-3.09 (m, 5H), 3.01 (s, 3H), 2.91 (dd, 1H), 2.78 (dd, 1H), 2.19-1.95 (m, 4H). LCMS (Method A): 534.3 [M+H]⁺.

### Example 288: N-((1S)-1-(6-((5-Chloro-2,3-dihydro-1H-inden-2-yl)amino)pyridin-3-yl)-2,2,2-trifluoroethyl)-N-methyltetrahydro-2H-thiopyran-4-carboxamide 1,1-dioxide—Isomer 1

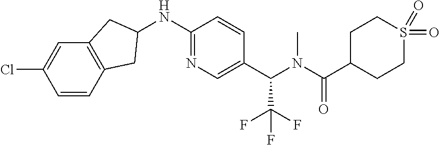 [SMILES: CN(C(=O)C1CCS(=O)(=O)CC1)[C@@H](c1ccc(NC2Cc3ccc(Cl)cc3C2)nc1)C(F)(F)F]

### Example 289: N-((1S)-1-(6-((5-Chloro-2,3-dihydro-1H-inden-2-yl)amino)pyridin-3-yl)-2,2,2-trifluoroethyl)-N-methyltetrahydro-2H-thiopyran-4-carboxamide 1,1-dioxide—Isomer 2

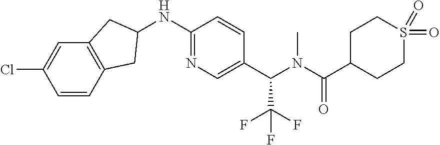 [SMILES: CN(C(=O)C1CCS(=O)(=O)CC1)[C@@H](c1ccc(NC2Cc3ccc(Cl)cc3C2)nc1)C(F)(F)F]

The title compound was prepared in an analogous manner to Intermediate 3 from Intermediate 169 (0.32 g, 0.58 mmol), pyridine (0.9 mL), tetrahydro-2H-thiopyran-4-carboxylic acid 1,1-dioxide (0.52 g, 2.92 mmol, CAS 64096-87-3), T3P® (4.16 mL, 14.0 mmol) at RT for 16 h. Purified by preparative HPLC (XBridge Phenyl, 19×250 mm×5 μm, flow rate: 18 mL/min, 10 mM aqueous NH₄HCO₃ with MeCN 40% to 55% over 9 min, held for 4 min, ramped to 100% over 0.1 min and held for 2.9 min). Chiral purification was carried out by preparative SFC (Chiralcel-OJ-H, 30×250 mm×5 μm, temp 30° C., Back Pressure 100 bar, flow rate: 100 g/min, 75% CO₂ with 25% MeOH modifier) to provide Example 288 (69 mg) as Peak 1, Example 289 (64 mg) as Peak 2. Example 288: ¹H NMR (400 MHz; DMSO-d₆) δ: 8.10-7.98 (m, 1H), 7.48-7.17 (m, 5H), 6.58-6.52 (m, 1H), 6.40-6.03 (m, 1H), 4.62-4.57 (m, 1H), 3.29-3.08 (m, 7H), 2.93 (s, 3H), 2.86-2.76 (m, 2H), 2.13-1.97 (m, 4H). LCMS (Method A): 516.3 [M+H]⁺. Example 289: ¹H NMR (400 MHz; DMSO-d₆) δ: 8.10-7.98 (m, 1H), 7.48-7.37 (m, 1H), 7.30 (s, 1H), 7.26-7.17 (m, 3H), 6.58-6.52 (m, 1H), 6.40-6.00 (m, 1H), 4.62-4.55 (m, 1H), 3.29-3.08 (m, 7H), 2.93 (s, 3H), 2.85-2.67 (m, 2H), 2.11-2.02 (m, 4H). LCMS (Method A): 516.3 [M+H]⁺.

### Example 290: N-((1S)-1-(5-((5-Chloro-4-fluoro-2,3-dihydro-1H-inden-2-yl)amino)pyridin-2-yl)-2,2,2-trifluoroethyl)-N-methyl-1-(methylsulfonamido)-cyclopropane-1-carboxamide

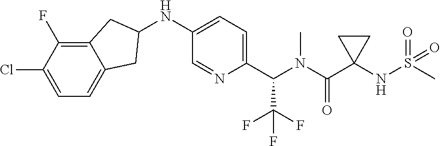 [SMILES: CN(C(=O)C1(NS(C)(=O)=O)CC1)[C@@H](c1ccc(NC2Cc3ccc(Cl)c(F)c3C2)cn1)C(F)(F)F]

The title compound (36 mg) was prepared in an analogous manner to Example 265 from Intermediate 183 (0.28 g, 0.76 mmol), Intermediate 204 (0.21 g, 1.15 mmol), AcOH (0.05 mL, 0.84 mmol) in MeOH (3.8 mL) at 50° C. for 3 h then sodium cyanoborohydride (0.19 g, 3.06 mmol) at RT for 16 h. Purified by preparative HPLC (Kinetex XB-C18, 21×250 mm×5 μm, flow rate: 30 mL/min, 0.1% aqueous formic acid with MeCN 40% to 60% over 6 min, held for 5 min, ramped to 95% over 0.2 min and held for 1.8 min). ¹H NMR (400 MHz; DMSO-d₆) δ: 8.09 (br s, 1H), 8.01 (d, 1H), 7.45-7.31 (m, 2H), 7.13 (d, 2H), 7.06-6.99 (m, 1H), 6.53 (d, 1H), 6.29-6.14 (m, 1H), 4.43-4.30 (m, 1H), 3.47-3.35 (m, 2H), 3.10 (br s, 2H), 2.89-2.82 (m, 4H), 2.84 (d, 1H), 1.31-1.07 (m, 4H). LCMS (Method M): 535.2 [M+H]⁺.

### Example 291: N-((1S)-1-(5-((5-Chloro-6-(difluoromethoxy)-2,3-dihydro-1H-inden-2-yl)amino)pyridin-2-yl)-2,2,2-trifluoroethyl)-N-methyltetrahydro-2H-thiopyran-4-carboxamide 1,1-dioxide

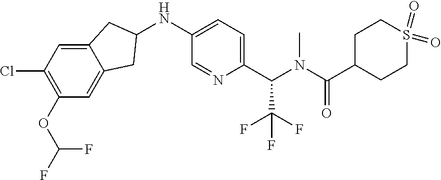 [SMILES: CN(C(=O)C1CCS(=O)(=O)CC1)[C@@H](c1ccc(NC2Cc3cc(Cl)c(OC(F)F)cc3C2)cn1)C(F)(F)F]

The title compound (30 mg) was prepared in an analogous manner to Example 265 from Intermediate 166 (70 mg, 0.19 mmol), Intermediate 174 (89 mg, 0.38 mmol), AcOH (57 mg, 0.96 mmol) in MeOH (1.3 mL) at 50° C. for 72 h then sodium cyanoborohydride (42 mg, 0.67 mmol) at RT for 17 h. Purified by preparative HPLC (XBridge C18, 19×150 mm×5 μm, flow rate: 30 mL/min, 0.05% aqueous ammonia with MeCN 10% to 60% over 5 min, held for 7 min, ramped to 95% over 0.2 min and held for 1.8 min). ¹H NMR (400 MHz; DMSO-d₆) δ: 8.01 (d, 1H), 7.48 (s, 1H), 7.30 (t, 1H), 7.28 (s, 1H), 7.15 (d, 1H), 7.04-7.00 (m, 1H), 6.51 (d, 1H), 6.38 (dd, 1H), 4.30 (dd, 1H), 3.26-3.08 (m, 6H), 3.02 (s, 3H), 2.84-2.80 (m, 2H), 2.21-1.94 (m, 5H). LCMS (Method M): 582.2 [M+H]⁺.

### Example 292: N¹-((1S)-1-(5-((5-Chloro-2,3-dihydro-1H-inden-2-yl)amino)pyridin-2-yl)-2,2,2-trifluoroethyl)-N¹,N⁴-dimethylcubane-1,4-dicarboxamide

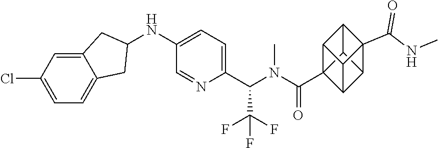 [SMILES: CNC(=O)C12C3C4C1C1C2C3C41C(=O)N(C)[C@@H](c1ccc(NC2Cc3ccc(Cl)cc3C2)cn1)C(F)(F)F]

The title compound (12 mg) was prepared in an analogous manner to Intermediate 3 from Intermediate 113 (27 mg, 0.07 mmol), pyridine (0.3 mL), Intermediate 176 (35 mg, 0.09 mmol), T3P® (0.24 mL, 0.40 mmol) at 50° C. for 72 h. Purified by preparative HPLC (Genesis NX-C18, 21×250 mm×5 μm, flow rate: 30 mL/min, water with MeCN 40% to 60% over 6 min, held for 4 min, ramped to 95% over 0.2 min and held for 2.8 min). ¹H NMR (400 MHz; DMSO-d₆) δ: 8.01 (d, 1H), 7.72 (d, 1H), 7.31 (d, 1H), 7.29-7.13 (m, 3H), 7.01 (dd, 1H), 6.49 (d, 1H), 6.30 (dd, 1H), 4.27 (dd, 1H), 4.21-4.07 (m, 6H), 3.27 (d, 2H), 2.86 (s, 3H), 2.84-2.73 (m, 2H), 2.59 (d, 3H). LCMS (Method T): 543.2 [M+H]⁺.

### Example 293: N-((1S)-1-(5-((4-Chloro-7-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

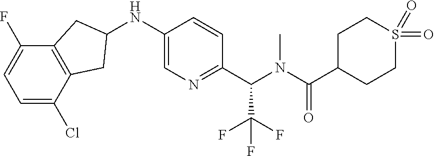 [SMILES: CN(C(=O)C1CCS(=O)(=O)CC1)[C@@H](c1ccc(NC2Cc3c(F)ccc(Cl)c3C2)cn1)C(F)(F)F]

### Example 294: N-((1S)-1-(5-((4-Chloro-7-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

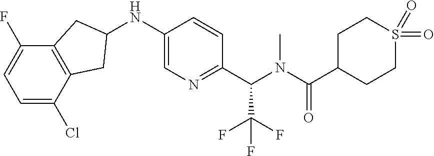 [SMILES: CN(C(=O)C1CCS(=O)(=O)CC1)[C@@H](c1ccc(NC2Cc3c(F)ccc(Cl)c3C2)cn1)C(F)(F)F]

The title compound was prepared in an analogous manner to Example 265 from Intermediate 166 (0.26 g, 0.68 mmol), 4-chloro-7-fluoro-1,3-dihydro-2H-inden-2-one (1.40 g, 1.37 mmol, CAS 1823959-98-3), AcOH (0.04 mL, 0.68 mmol) in MeOH (5 mL) at 60° C. for 6 h then sodium cyanoborohydride (86 mg, 1.36 mmol) at RT for 16 h. Purified by flash chromatography (silica gel, eluting 0-70% EtOAc in petroleum ether). Chiral purification was carried out by preparative SFC (Lux-Cellulose-2, 30×250 mm×5 μm, temp 30° C., Back Pressure 100 bar, flow rate: 100 g/min, 70% CO₂ with 30% MeOH modifier) to provide Example 293 (38 mg) as Peak 1, Example 294 (38 mg) as Peak 2. Example 293: ¹H NMR (400 MHz; DMSO-d₆) δ: 8.06-8.01 (m, 1H), 7.32-7.26 (m, 1H), 7.15 (d, 1H), 7.10 (d, 1H), 7.07-7.02 (m, 1H), 6.63-6.57 (m, 1H), 6.42-6.02 (m 1H), 4.39-4.36 (m, 1H), 3.48-3.31 (m, 2H), 3.24-3.08 (m, 5H), 3.01 (s, 3H), 2.93-2.83 (m, 2H), 2.16-1.95 (m, 4H). LCMS (Method A): 534.4 [M+H]⁺. Example 294: ¹H NMR (400 MHz; DMSO-d₆) δ: 8.06-8.01 (m, 1H), 7.32-7.26 (m, 1H), 7.16 (d, 1H), 7.08 (d, 1H), 7.06-7.02 (m, 1H), 6.63-6.57 (m, 1H), 6.42-6.03 (m, 1H), 4.38-4.36 (m, 1H), 3.47-3.37 (m, 2H), 3.27-3.09 (m, 5H), 3.01 (s, 3H), 2.93-2.83 (m, 2H), 2.16-1.95 (m, 4H). LCMS (Method A): 534.4 [M+H]⁺.

### Example 295: N-Methyl-N-((1S)-2,2,2-trifluoro-1-(5-((4,5,6-trifluoro-2,3-dihydro-1H-inden-2-yl)amino)pyridin-2-yl)ethyl)tetrahydro-2H-thiopyran-4-carboxamide 1,1-dioxide—Isomer 1

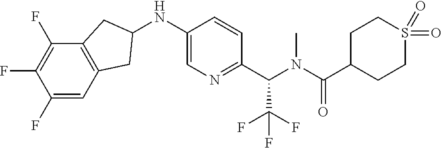 [SMILES: CN(C(=O)C1CCS(=O)(=O)CC1)[C@@H](c1ccc(NC2Cc3cc(F)c(F)c(F)c3C2)cn1)C(F)(F)F]

### Example 296: N-Methyl-N-((1S)-2,2,2-trifluoro-1-(5-((4,5,6-trifluoro-2,3-dihydro-1H-inden-2-yl)amino)pyridin-2-yl)ethyl)tetrahydro-2H-thiopyran-4-carboxamide 1,1-dioxide—Isomer 2

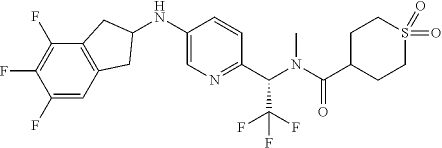 [SMILES: CN(C(=O)C1CCS(=O)(=O)CC1)[C@@H](c1ccc(NC2Cc3cc(F)c(F)c(F)c3C2)cn1)C(F)(F)F]

The title compound was prepared in an analogous manner to Intermediate 3 from Intermediate 180 (0.13 g, 0.29 mmol), pyridine (3.9 mL), tetrahydro-2H-thiopyran-4-carboxylic acid 1,1-dioxide (0.16 g, 0.88 mmol, CAS 64096-87-3), T3P® (20 mL, 67.2 mmol) at RT for 16 h. Purified by preparative HPLC (Gemini NX-C18, 21×250 mm×5 μm, flow rate: 18 mL/min, 10 mM aqueous NH₄HCO₃ with MeCN 50% to 82% over 7 min, held for 1.75 min, ramped to 95% over 0.2 min and held for 3 min). Chiral purification was carried out by preparative SFC (Chiralpak®-AD-H, 30×250 mm×5 μm, temp 30° C., Back Pressure 100 bar, flow rate: 100 g/min, 60% CO₂ with 40% MeOH modifier) to provide Example 295 (33 mg) as Peak 1, Example 296 (30 mg) as Peak 2. Example 295: ¹H NMR (400 MHz; DMSO-d₆) δ: 8.04-8.00 (m, 1H), 7.26-7.15 (m, 1H), 7.11 (d, 1H), 7.07-7.01 (m, 1H), 6.59-6.53 (m, 1H), 6.41-6.04 (m, 1H), 4.37-4.33 (m, 1H), 3.42-3.23 (m, 2H), 3.21-3.09 (m, 5H), 3.01 (s, 3H), 2.86-2.67 (m, 2H), 2.15-1.94 (m, 4H). LCMS (Method A): 536.3 [M+H]⁺. Example 296: ¹H NMR (400 MHz; DMSO-d₆) δ: 8.04-8.00 (m, 1H), 7.27-7.15 (m, 1H), 7.15 (d, 1H), 7.08-7.01 (m, 1H), 6.59-6.53 (m, 1H), 6.41-6.02 (m, 1H), 4.37-4.34 (m, 1H), 3.41-3.08 (m, 7H), 3.01 (s, 3H), 2.85-2.80 (m, 2H), 2.16-1.94 (m, 4H). LCMS (Method A): 536.3 [M+H]⁺.

### Example 297: (S)—N—((S)-1-(5-(((S)-5-Chloro-2,3-dihydro-1H-inden-2-yl)amino)pyridin-2-yl)-2,2,2-trifluoroethyl)-N-methyl-6-oxopiperidine-3-carboxamide

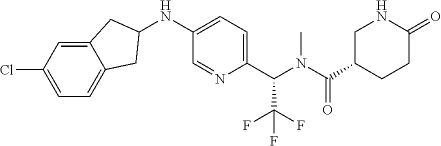 [SMILES: CN(C(=O)[C@H]1CCC(=O)NC1)[C@@H](c1ccc(NC2Cc3ccc(Cl)cc3C2)cn1)C(F)(F)F]

The title compound (17 mg) was prepared in an analogous manner to Intermediate 3 from Intermediate 187 (60 mg, 0.17 mmol), pyridine (0.6 mL), (3S)-6-oxopiperidine-3-carboxylic acid (27 mg, 0.19 mmol, CAS 1426408-56-1), T3P® (50% in MeTHF, 0.31 mL, 0.49 mmol) at RT for 32 h. Extra (3S)-6-oxopiperidine-3-carboxylic acid (6 mg, 0.04 mmol, CAS 1426408-56-1), T3P® (50% in MeTHF, 0.21 mL, 0.34 mmol) were added and stirred at RT for 4 h. Purified by preparative HPLC (Genesis NX-C18, 21×250 mm×5 μm, flow rate: 30 mL/min, 0.1% aqueous formic acid with MeCN 40% to 50% over 6 min, held for 8 min, ramped to 95% over 0.2 min and held for 1.8 min). ¹H NMR (400 MHz; DMSO-d₆) δ: 8.02 (s, 1H), 7.49 (s, 1H), 7.32 (d, 1H), 7.27 (d, 1H), 7.21 (dd, 1H), 7.13 (d, 1H), 7.08-6.94 (m, 1H), 6.49 (d, 1H), 6.43-6.35 (m, 1H), 4.28 (dd, 1H), 3.30-3.11 (m, 5H), 3.03 (s, 3H), 2.85-2.65 (m, 2H), 2.30-2.14 (m, 2H), 1.94-1.69 (m, 2H). LCMS (Method M): 481.2 [M+H]⁺.

### Example 298: (S)—N-(1-(5-((5,6-Dichloro-2,3-dihydro-1H-inden-2-yl)amino)pyridin-2-yl)-2,2,2-trifluoroethyl)-N-methyltetrahydro-2H-thiopyran-4-carboxamide 1,1-dioxide

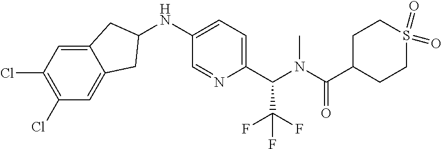 [SMILES: CN(C(=O)C1CCS(=O)(=O)CC1)[C@@H](c1ccc(NC2Cc3cc(Cl)c(Cl)cc3C2)cn1)C(F)(F)F]

The title compound (15 mg) was prepared in an analogous manner to Example 265 from Intermediate 166 (68 mg, 0.19 mmol), 5,6-dichloro-1,3-dihydro-2H-inden-2-one (46 mg, 0.22 mmol, CAS 1187983-77-2), AcOH (0.02 mL, 0.40 mmol) in MeOH (1.9 mL) at 60° C. for 6 h then sodium cyanoborohydride (19 mg, 0.30 mmol) and 5,6-dichloro-1,3-dihydro-2H-inden-2-one (37 mg, 0.18 mmol, CAS 1187983-77-2), at RT for 42 h. Purified by flash chromatography (silica gel, eluting 10-100% EtOAc in cyclohexane). ¹H NMR (400 MHz; DMSO-d₆) δ: 8.01 (d, 1H), 7.53 (s, 2H), 7.15 (d, 1H), 7.04 (dd, 1H), 6.52 (d, 1H), 6.38 (dd, 1H), 4.30 (dd, 1H), 3.31-3.07 (m, 7H), 3.01 (s, 3H), 2.80 (dd, 2H), 2.14-1.89 (m, 4H). LCMS (Method M): 550.2 [M+H]⁺.

### Example 299: N—((S)-1-(5-(((S)-5-Chloro-2,3-dihydro-1H-inden-2-yl)amino)pyridin-2-yl)-2,2,2-trifluoroethyl)-N-methyl-3-(methylsulfonamido)bicyclo[1.1.1]pentane-1-carboxamide

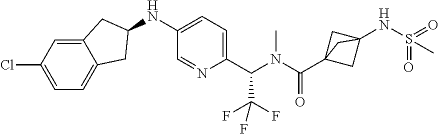 [SMILES: CN(C(=O)C12CC(NS(C)(=O)=O)(C1)C2)[C@@H](c1ccc(N[C@H]2Cc3ccc(Cl)cc3C2)cn1)C(F)(F)F]

The title compound (26 mg) was prepared in an analogous manner to Example 169 from Intermediate 189 (77 mg, 0.16 mmol), triethylamine (69 μL, 0.49 mmol) and methane sulfonyl chloride (15 μL, 0.20 mmol) in DCM (1.2 mL) at RT for 16 h. Purified by preparative HPLC (Gemini NX-C18, 21×250 mm×5 μm, flow rate: 30 mL/min, 0.05% aqueous ammonia with MeCN 50% to 65% over 6 min, held for 2 min, ramped to 95% over 0.2 min and held for 1.8 min). ¹H NMR (400 MHz; CDCl₃) δ: 8.05 (d, 1H), 7.25 (s, 1H), 7.24-7.14 (m, 3H), 6.91 (dd, 1H), 6.45 (dd, 1H), 4.93 (s, 1H), 4.10 (dd, 1H), 3.44-3.33 (m, 2H), 3.13-3.11 (m, 2H), 3.03 (s, 3H), 2.93-2.84 (m, 2H), 2.83 (s, 1H), 2.60-2.48 (m, 6H), 2.20 (s, 1H). LCMS (Method M): 543.1 [M+H]⁺.

### Example 300: (S)—N—((S)-1-(5-(((S)-5-Chloro-2,3-dihydro-1H-inden-2-yl)amino)pyridin-2-yl)-2,2,2-trifluoroethyl)-N-methyl-5-oxopyrrolidine-3-carboxamide

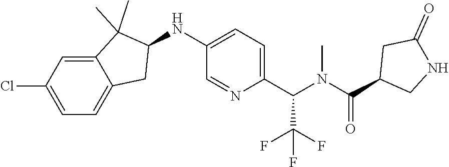 [SMILES: CN(C(=O)[C@@H]1CNC(=O)C1)[C@@H](c1ccc(N[C@H]2Cc3ccc(Cl)cc3C2(C)C)cn1)C(F)(F)F]

The title compound (20 mg) was prepared in an analogous manner to Intermediate 3 from Intermediate 187 (60 mg, 0.17 mmol), pyridine (0.6 mL), (3S)-5-oxopyrrolidine-3-carboxylic acid (24 mg, 0.19 mmol, CAS 30948-17-5), T3P® (50% in MeTHF, 0.54 mL, 0.84 mmol) at RT for 16 h. Purified by preparative HPLC (Genesis NX-C18, 21×250 mm×5 μm, flow rate: 30 mL/min, 0.1% aqueous formic acid with MeCN 30% to 60% over 10 min, ramped to 95% over 0.2 min and held for 1.8 min). ¹H NMR (400 MHz; DMSO-d₆) δ: 8.02 (d, 1H), 7.65 (d, 1H), 7.36-7.10 (m, 4H), 7.03 (d, 1H), 6.50 (d, 1H), 6.38 (dd, 1H), 4.27 (dd, 1H), 3.79-3.67 (m, 1H), 3.56 (dd, 1H), 3.35 (s, 1H), 3.28-3.24 (m, 2H), 2.96 (s, 3H), 2.86-2.71 (m, 2H), 2.43-2.28 (m, 2H). LCMS (Method M): 467.2 [M+H]⁺.

### Example 301: (R)—N—((S)-1-(5-(((S)-5-Chloro-2,3-dihydro-1H-inden-2-yl)amino)pyridin-2-yl)-2,2,2-trifluoroethyl)-N,3-dimethyl-2-oxoimidazolidine-4-carboxamide

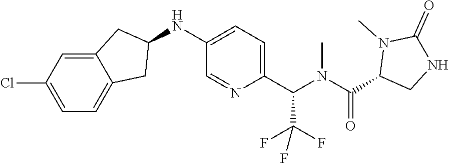 [SMILES: CN1C(=O)NC[C@@H]1C(=O)N(C)[C@@H](c1ccc(N[C@H]2Cc3ccc(Cl)cc3C2)cn1)C(F)(F)F]

The title compound (6 mg) was prepared in an analogous manner to Intermediate 3 from Intermediate 187 (60 mg, 0.17 mmol), pyridine (0.8 mL), (4R)-3-methyl-2-oxo-imidazolidine-4-carboxylic acid (30 mg, 0.18 mmol, CAS 1426408-63-0), T3P® (50% in MeTHF, 0.30 mL, 0.51 mmol) at RT for 16 h. Purified by preparative HPLC (Genesis NX-C18, 21×250 mm×5 μm, flow rate: 30 mL/min, 0.1% aqueous formic acid with MeCN 50% isocratic over 14 min, ramped to 95% over 0.2 min and held for 1.8 min). ¹H NMR (400 MHz; DMSO-d₆) δ: 8.02 (d, 1H), 7.33-7.13 (m, 4H), 7.03 (dd, 1H), 6.53 (d, 1H), 6.41-6.30 (m, 2H), 4.75 (dd, 1H), 4.32-4.24 (m, 1H), 3.59 (dd, 1H), 3.29-3.25 (m, 2H), 2.93 (s, 3H), 2.84-2.76 (m, 3H), 2.62 (s, 3H). LCMS (Method M): 482.2 [M+H]⁺.

### Example 302: (S)—N-(1-(5-((4,7-Dichloro-2,3-dihydro-1H-inden-2-yl)amino)pyridin-2-yl)-2,2,2-trifluoroethyl)-N-methyltetrahydro-2H-thiopyran-4-carboxamide 1,1-dioxide

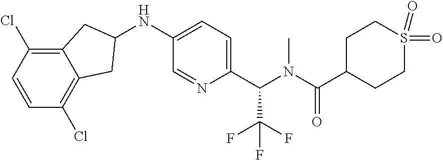 [SMILES: CN(C(=O)C1CCS(=O)(=O)CC1)[C@@H](c1ccc(NC2Cc3c(Cl)ccc(Cl)c3C2)cn1)C(F)(F)F]

The title compound (13 mg) was prepared in an analogous manner to Example 265 from Intermediate 166 (61 mg, 0.15 mmol), 4,7-dichloro-1,3-dihydro-2H-inden-2-one (60 mg, 0.29 mmol, CAS 1823959-93-8), AcOH (43 μL, 0.08 mmol) in MeOH (0.9 mL) at 50° C. for 16 h then sodium cyanoborohydride (39 mg, 0.62 mmol) and molecular sieves at RT for 72 h. Purified by preparative HPLC (Genesis NX-C18, 21×250 mm×5 μm, flow rate: 30 mL/min, 0.1% aqueous formic acid with MeCN 40% to 55% over 6 min, held for 5 min, ramped to 95% over 0.2 min and held for 1.8 min). ¹H NMR (400 MHz; DMSO-d₆) δ: 8.04 (d, 1H), 7.32 (s, 2H), 7.17 (d, 1H), 7.12-7.00 (m, 1H), 6.61 (d, 1H), 6.39 (dd, 1H), 4.43-4.29 (m, 1H), 3.47 (dd, 2H), 3.28-3.09 (m, 5H), 3.02 (s, 3H), 2.92 (dd, 2H), 2.23-1.90 (m, 4H). LCMS (Method M): 550.1 [M+H]⁺.

### Example 303: N—((S)-1-(5-(((S)-5-Chloro-2,3-dihydro-1H-inden-2-yl)amino)pyridin-2-yl)-2,2,2-trifluoroethyl)-N-methyl-2-oxooxazolidine-5-carboxamide

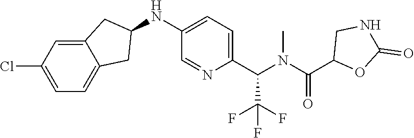 [SMILES: CN(C(=O)C1CNC(=O)O1)[C@@H](c1ccc(N[C@H]2Cc3ccc(Cl)cc3C2)cn1)C(F)(F)F]

The title compound (27 mg) was prepared in an analogous manner to Intermediate 3 from Intermediate 187 (50 mg, 0.14 mmol), pyridine (0.7 mL), 2-oxooxazolidine-5-carboxylic acid (20 mg, 0.16 mmol, CAS 60487-08-3), T3P® (50% in MeTHF, 0.42 mL, 0.70 mmol) at RT for 16 h. Purified by preparative HPLC (Genesis NX-C18, 21×250 mm×5 μm, flow rate: 30 mL/min, 0.05% aqueous ammonia with MeCN 30% to 65% over 6 min, held for 5 min, ramped to 95% over 0.2 min and held for 1.8 min). ¹H NMR (300 MHz; DMSO-d₆) δ: 8.03 (d, 1H), 7.78 (dd, 1H), 7.37-7.13 (m, 4H), 7.11-6.99 (m, 1H), 6.54 (dd, 1H), 6.41-6.23 (m, 1H), 5.60-5.54 (m, 1H), 4.39-4.18 (m, 1H), 3.80-3.72 (m, 1H), 3.54-3.48 (m, 1H), 3.39-3.23 (m, 2H), 3.01-2.94 (m, 3H), 2.87-2.73 (m, 2H). LCMS (Method M): 469.1 [M+H]⁺.

### Example 304: (R)—N—((S)-1-(5-(((S)-5-Chloro-2,3-dihydro-1H-inden-2-yl)amino)pyridin-2-yl)-2,2,2-trifluoroethyl)-N-methyl-5-oxopyrrolidine-2-carboxamide Hydrochloride

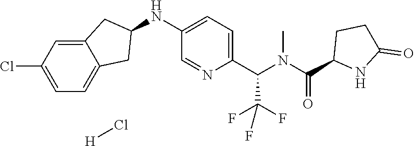 [SMILES: CN(C(=O)[C@H]1CCC(=O)N1)[C@@H](c1ccc(N[C@H]2Cc3ccc(Cl)cc3C2)cn1)C(F)(F)F ; Cl]

To a stirred solution of Intermediate 190 (14 mg, 24.4 μmol) in 1,4-dioxane (0.1 mL) was added 4M HCl in 1,4-dioxane (0.1 mL) at RT for 2 h. The mixture concentrated under reduced pressure. The crude was dissolved in DCM then 2M HCl in diethyl ether (87 μL) and stirred at RT for 20 h. The mixture was concentrated under reduced pressure to provide the title compound (11 mg). ¹H NMR (300 MHz; DMSO-d₆) δ: 8.02 (d, 1H), 7.91 (s, 1H), 7.36-7.11 (m, 4H), 7.03 (dd, 1H), 6.53 (br s, 1H), 6.34 (dd, 1H), 4.64 (dd, 1H), 4.36-4.15 (m, 1H), 3.35-3.23 (m, 3H), 2.96 (s, 3H), 2.87-2.75 (m, 2H), 2.45-2.29 (m, 2H), 2.19-2.05 (m, 2H). LCMS (Method M): 467.2 [M+H]⁺.

### Example 305: N-((1S)-1-(5-((5-Chloro-4-cyclopropyl-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

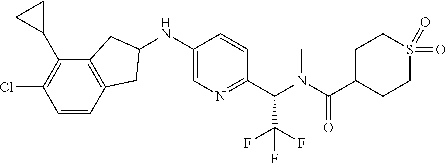 [SMILES: CN(C(=O)C1CCS(=O)(=O)CC1)[C@@H](c1ccc(NC2Cc3ccc(Cl)c(C4CC4)c3C2)cn1)C(F)(F)F]

### Example 306: N-((1S)-1-(5-((5-Chloro-4-cyclopropyl-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

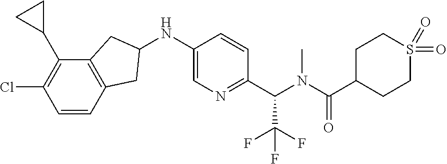 [SMILES: CN(C(=O)C1CCS(=O)(=O)CC1)[C@@H](c1ccc(NC2Cc3ccc(Cl)c(C4CC4)c3C2)cn1)C(F)(F)F]

The title compound was prepared in an analogous manner to Example 265 from Intermediate 166 (0.10 g, 0.27 mmol), Intermediate 193 (0.11 g, 0.54 mmol), AcOH (0.08 mL, 0.14 mmol) in MeOH (1.8 mL) at 50° C. for 16 h then sodium cyanoborohydride (73 mg, 1.16 mmol) at RT for 72 h. Purified by preparative HPLC (Genesis NX-C18, 21×250 mm×5 μm, flow rate: 30 mL/min, 0.1% aqueous formic acid with MeCN 40% to 55% over 6 min, held for 5 min, ramped to 95% over 0.2 min and held for 1.8 min). Chiral purification was carried out by preparative SFC (Lux-i-Amylose-3, 30×250 mm×5 μm, temp 30° C., Back Pressure 100 bar, flow rate: 100 g/min, 70% CO₂ with 30% MeOH modifier) to provide Example 305 (11 mg) as Peak 1, Example 306 (11 mg) as Peak 3. Example 305: ¹H NMR (400 MHz; DMSO-d₆) δ: 8.04-8.01 (m, 1H), 7.28-7.00 (m, 4H), 6.52-6.46 (m, 1H), 6.40-6.03 (m, 1H), 4.24 (dd, 1H), 3.44 (dd, 1H), 3.27-3.08 (m, 6H), 3.02 (s, 3H), 2.90 (dd, 1H), 2.77 (dd, 1H), 2.08-2.01 (m, 4H), 1.81 (dd, 1H), 0.97 (dd, 2H), 0.64-0.61 (m, 2H). LCMS (Method A): 556.3 [M+H]⁺. Example 306: ¹H NMR (400 MHz; DMSO-d₆) δ: 8.05-8.01 (m, 1H), 7.27-7.00 (m, 4H), 6.52-6.46 (m, 1H), 6.40-6.03 (m, 1H), 4.25 (dd, 1H), 3.44 (dd, 1H), 3.25-3.08 (m, 6H), 3.02 (s, 3H), 2.94-2.87 (m, 1H), 2.78 (dd, 1H), 2.07-1.99 (m, 4H), 1.98-1.81 (m, 1H), 0.98-0.95 (m, 2H), 0.65-0.61 (m, 2H). LCMS (Method A): 556.3 [M+H]⁺.

### Example 307: N-((1S)-1-(5-((5-Cyclopropyl-2,3-dihydro-1H-inden-2-yl)amino)pyridin-2-yl)-2,2,2-trifluoroethyl)-N-methyltetrahydro-2H-thiopyran-4-carboxamide 1,1-dioxide

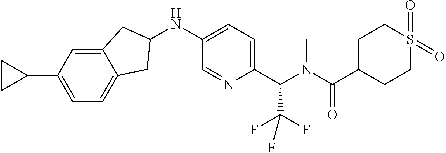 [SMILES: CN(C(=O)C1CCS(=O)(=O)CC1)[C@@H](c1ccc(NC2Cc3ccc(C4CC4)cc3C2)cn1)C(F)(F)F]

The title compound (14 mg) was prepared in an analogous manner to Intermediate 193 from Example 284 (40 mg, 0.07 mmol), potassium cyclopropyltrifluoroborate (21 mg, 0.14 mmol, CAS 1065010-87-8), Pd(dppf)Cl₂ (5.2 mg, 0.07 mmol) and K₃PO₄ (45 mg, 0.14 mmol) in toluene (0.5 mL) and water (0.1 ml) at 90° C. for 16 h. Purified by preparative HPLC (XBridge C18, 19×150 mm×5 μm, flow rate: 30 mL/min, 0.1% aqueous formic acid with MeCN 10% to 65% over 5 min, held for 4 min, ramped to 95% over 0.2 min and held for 1.8 min). ¹H NMR (300 MHz; DMSO-d₆) δ: 8.02 (d, 1H), 7.25-6.80 (m, 5H), 6.52-6.32 (m, 2H), 4.23 (dd, 1H), 3.29-3.06 (m, 7H), 3.02 (s, 3H), 2.79-2.69 (m, 3H), 2.07-1.81 (m, 4H), 0.96-0.84 (m, 2H), 0.66-0.56 (m, 2H). LCMS (Method M): 522.2 [M+H]⁺.

### Example 308: N-((1S)-1-(5-((5-Cyclopropyl-2,3-dihydro-1H-inden-2-yl)amino)pyridin-2-yl)-2,2,2-trifluoroethyl)-N-methyltetrahydro-2H-thiopyran-4-carboxamide 1,1-dioxide

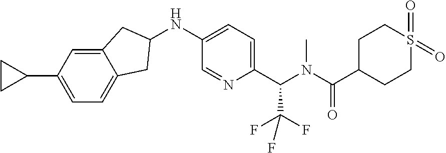 [SMILES: CN(C(=O)C1CCS(=O)(=O)CC1)[C@@H](c1ccc(NC2Cc3ccc(C4CC4)cc3C2)cn1)C(F)(F)F]

The title compound (26 mg) was prepared in an analogous manner to Intermediate 193 from Example 285 (40 mg, 0.07 mmol), potassium cyclopropyltrifluoroborate (21 mg, 0.14 mmol, CAS 1065010-87-8), Pd(dppf)Cl₂ (5.2 mg, 0.07 mmol) and K₃PO₄ (45 mg, 0.14 mmol) in toluene (0.5 mL) and water (0.1 ml) at 90° C. for 16 h. Purified by preparative HPLC (Gemini-NX C18, 21×250 mm×5 μm, flow rate: 30 mL/min, 0.1% aqueous formic acid with MeCN 50% to 60% over 6 min, held for 5 min, ramped to 95% over 0.2 min and held for 1.8 min). ¹H NMR (400 MHz; 353 K, DMSO-d₆) δ: 8.05 (d, 1H), 7.22-7.08 (m, 2H), 7.07-7.00 (m, 1H), 6.96 (s, 1H), 6.90 (dd, 1H), 6.38 (br s, 1H), 6.19 (d, 1H), 4.27 (dd, 1H), 3.30-3.10 (m, 6H), 3.04 (s, 3H), 2.79 (ddd, 2H), 2.19-2.00 (m, 4H), 1.90 (dd, 1H), 0.96-0.83 (m, 2H), 0.68-0.55 (m, 2H)—1H obscured by water signal. LCMS (Method M): 522.3 [M+H]⁺.

### Example 309: (R)—N—((S)-1-(5-(((S)-5-Chloro-2,3-dihydro-1H-inden-2-yl)amino)pyridin-2-yl)-2,2,2-trifluoroethyl)-N-methyl-6-oxopiperidine-2-carboxamide

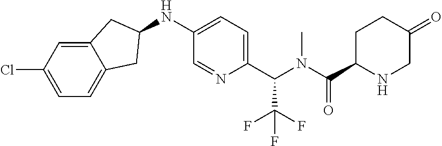 [SMILES: CN(C(=O)[C@H]1CCC(=O)CN1)[C@@H](c1ccc(N[C@H]2Cc3ccc(Cl)cc3C2)cn1)C(F)(F)F]

The title compound (1.6 mg) was prepared in an analogous manner to Intermediate 3 from Intermediate 187 (36 mg, 0.10 mmol), pyridine (0.8 mL), Intermediate 194 (0.22 g, 0.10 mmol), T3P® (50% in MeTHF, 0.36 mL, 0.61 mmol) at RT for 42 h. Purified by preparative HPLC (Genesis NX-C18, 21×250 mm×5 μm, flow rate: 30 mL/min, 0.05% aqueous ammonia with MeCN 30% to 70% over 6 min, held for 5 min, ramped to 95% over 0.2 min and held for 1.8 min). ¹H NMR (400 MHz; 353 K, DMSO-d₆) δ: 8.04 (d, 1H), 7.28 (d, 1H), 7.25 (d, 1H), 7.20-7.08 (m, 3H), 7.04 (dd, 1H), 6.30 (br s, 1H), 6.24-6.17 (m, 1H), 4.57 (br s, 1H), 4.38-4.16 (m, 1H), 3.40-2.28 (m, 2H), 2.98 (s, 3H), 2.89-2.78 (m, 2H), 2.18-2.14 (m, 2H), 1.99-1.88 (m, 1H), 1.83-1.56 (m, 3H). LCMS (Method M): 481.2 [M+H]⁺.

### Example 310: N-((1S)-1-(5-((5-Bromo-6-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide

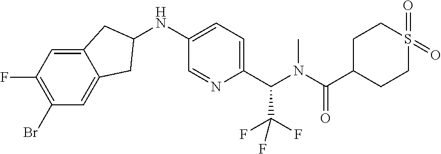 [SMILES: CN(C(=O)C1CCS(=O)(=O)CC1)[C@@H](c1ccc(NC2Cc3cc(F)c(Br)cc3C2)cn1)C(F)(F)F]

The title compound (0.20 g) was prepared in an analogous manner to Example 265 from Intermediate 166 (0.20 g, 0.55 mmol), Intermediate 196 (0.25 g, 1.10 mmol), AcOH (0.21 g, 3.28 mmol) in MeOH (3.6 mL) at 50° C. for 16 h then sodium cyanoborohydride (39 mg, 0.62 mmol) and molecular sieves at RT for 16 h. Purified by automated flash chromatography (40 g silica gel, eluting 0-45% EtOAc in cyclohexane). ¹H NMR (400 MHz; DMSO-d₆) δ: 8.04-8.00 (m, 1H), 7.57 (d, 1H), 7.29 (d, 1H), 7.15 (d, 1H), 7.08-7.00 (m, 1H), 6.50 (d, 1H), 6.38 (dd, 1H), 4.30 (dd, 1H), 3.32-3.07 (m, 7H), 3.02 (s, 3H), 2.79 (dd, 2H), 2.18-1.92 (m, 4H). LCMS (Method M): 580.1 [M+H]⁺.

### Example 311: N-((1S)-1-(5-((5-Chloro-4-(trifluoromethyl)-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

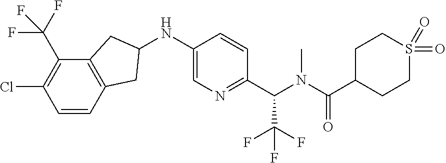 [SMILES: CN(C(=O)C1CCS(=O)(=O)CC1)[C@@H](c1ccc(NC2Cc3ccc(Cl)c(C(F)(F)F)c3C2)cn1)C(F)(F)F]

### Example 312: N-((1S)-1-(5-((5-Chloro-4-(trifluoromethyl)-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

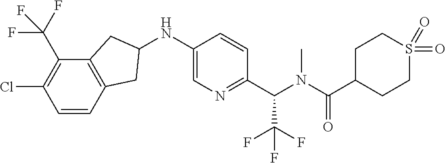 [SMILES: CN(C(=O)C1CCS(=O)(=O)CC1)[C@@H](c1ccc(NC2Cc3ccc(Cl)c(C(F)(F)F)c3C2)cn1)C(F)(F)F]

The title compound was prepared in an analogous manner to Example 265 from Intermediate 166 (0.26 g, 0.68 mmol), Intermediate 199 (0.18 g, 0.75 mmol), AcOH (41 mg, 0.68 mmol) in MeOH (15 mL) at 60° C. for 6 h then sodium cyanoborohydride (0.13 g, 2.05 mmol) at 60° C. for 6 h. Purified by flash chromatography (silica gel, eluting 0-70% EtOAc in petroleum ether). Chiral purification was carried out by preparative SFC (Chiralpak®-IG, 30×250 mm×5 μm, temp 30° C., Back Pressure 100 bar, flow rate: 100 g/min, 50% CO₂ with 50% MeOH modifier) to provide Example 311 (21 mg) as Peak 1, Example 312 (28 mg) as Peak 2. Example 311: ¹H NMR (400 MHz; DMSO-d₆) δ: 8.04-8.00 (m, 1H), 7.55 (d, 1H), 7.51 (dd 1H), 7.27-7.13 (m, 1H), 7.08-7.01 (m, 1H), 6.57-6.51 (m, 1H), 6.41-6.01 (m, 1H), 4.30 (dd, 1H), 3.62-3.56 (m, 1H), 3.24-3.09 (m, 7H), 3.01 (s, 3H), 2.86 (dd, 1H), 2.18-1.94 (m, 4H). LCMS (Method R): 584.3 [M+H]⁺. Example 312: ¹H NMR (400 MHz; DMSO-d₆) δ: 8.04-8.00 (m, 1H), 7.55 (d, 1H), 7.51 (d, 1H), 7.28-7.13 (m, 1H), 7.08-7.01 (m, 1H), 6.57-6.51 (m, 1H), 6.41-6.01 (m, 1H), 4.30 (dd, 1H), 3.62-3.56 (m, 1H), 3.24-3.14 (m, 7H), 3.01 (s, 3H), 2.85 (dd, 1H), 2.18-1.94 (m, 4H). LCMS (Method R): 584.3 [M+H]⁺.

### Example 313: N—((S)-1-(5-(((S)-5-Chloro-2,3-dihydro-1H-inden-2-yl)amino)pyridin-2-yl)-2,2,2-trifluoroethyl)-N-methyl-2-oxopiperidine-4-carboxamide—Isomer 1

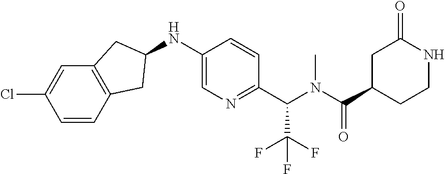 [SMILES: CN(C(=O)[C@@H]1CCNC(=O)C1)[C@@H](c1ccc(N[C@H]2Cc3ccc(Cl)cc3C2)cn1)C(F)(F)F]

### Example 314: N—((S)-1-(5-(((S)-5-Chloro-2,3-dihydro-1H-inden-2-yl)amino)pyridin-2-yl)-2,2,2-trifluoroethyl)-N-methyl-2-oxopiperidine-4-carboxamide—Isomer 2

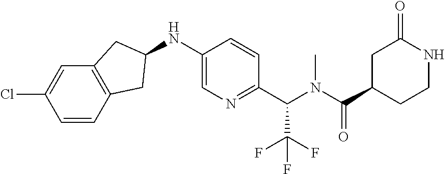 [SMILES: CN(C(=O)[C@@H]1CCNC(=O)C1)[C@@H](c1ccc(N[C@H]2Cc3ccc(Cl)cc3C2)cn1)C(F)(F)F]

The title compound was prepared in an analogous manner to Intermediate 3 from Intermediate 187 (0.10 g, 0.28 mmol), pyridine (1.4 mL), 2-oxopiperidine-4-carboxylic acid (44 mg, 0.31 mmol, CAS 24537-50-6), T3P® (0.50 mL, 0.84 mmol) at RT for 16 h. Purified by preparative HPLC (Gemini NX-C18, 21×250 mm×5 μm, flow rate: 30 mL/min, 0.1% aqueous formic acid with MeCN 30% to 60% over 8 min, held for 3 min, ramped to 95% over 0.2 min and held for 1.8 min). Chiral purification was carried out by preparative SFC (Chiralpak®-AD-H, 30×250 mm×5 μm, temp 30° C., Back Pressure 100 bar, flow rate: 100 g/min, 60% CO₂ with 40% MeOH modifier) to provide Example 313 (9 mg) as Peak 1, Example 314 (10 mg) as Peak 2. Example 313: ¹H NMR (400 MHz; DMSO-d₆) δ: 8.01 (d, 1H), 7.50-7.46 (m, 1H), 7.31 (s, 1H), 7.27-7.25 (m, 1H), 7.23-7.21 (m, 1H), 7.16-7.13 (m, 1H), 7.07-7.00 (m, 1H), 6.51-6.47 (m, 1H), 6.43-6.07 (m, 1H), 4.27 (dd, 1H), 3.26-2.83 (m, 5H), 3.00 (s, 3H), 2.84-2.76 (m, 2H), 2.29-2.15 (m, 2H), 1.92-1.88 (m, 1H), 1.71-1.65 (m, 1H). LCMS (Method A): 481.3 [M+H]⁺. Example 314: ¹H NMR (400 MHz; DMSO-d₆) δ: 8.04-7.97 (m, 1H), 7.53-7.50 (m, 1H), 7.43-7.18 (m, 3H), 7.07-7.00 (m, 2H), 6.52-6.47 (m, 1H), 6.41-6.10 (m, 1H), 4.28 (dd, 1H), 3.27-3.12 (m, 5H), 3.00 (s, 3H), 2.93-2.67 (m, 2H), 2.26-2.22 (m, 2H), 1.84-1.62 (m, 2H). LCMS (Method A): 481.4 [M+H]⁺.

### Example 315: (S)—N—((S)-1-(5-(((S)-5-Chloro-2,3-dihydro-1H-inden-2-yl)amino)pyridin-2-yl)-2,2,2-trifluoroethyl)-N-methyl-5-oxopyrrolidine-2-carboxamide

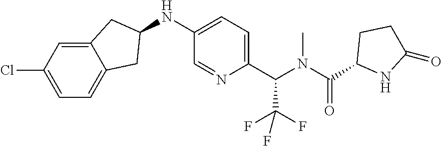 [SMILES: CN(C(=O)[C@@H]1CCC(=O)N1)[C@@H](c1ccc(N[C@H]2Cc3ccc(Cl)cc3C2)cn1)C(F)(F)F]

To a stirred solution of Intermediate 200 (28 mg, 47 μmol) in DCM (0.2 mL) was added TFA (0.04 mL) at RT for 1 h. The mixture concentrated under reduced pressure. The crude was purified by SPE (InertSep NH2 2 g, eluting 2% MeOH in DCM) to provide the title compound (16 mg). ¹H NMR (400 MHz; 353 K, DMSO-d₆) δ: 8.05 (d, 1H), 7.47 (s, 1H), 7.31-7.22 (m, 2H), 7.22-7.13 (m, 2H), 7.04 (dd, 1H), 6.36 (br s, 1H), 6.23 (d, 1H), 4.64 (br s, 1H), 4.64-4.28 (m, 1H), 3.36-3.30 (m, 2H), 2.98 (s, 3H), 2.83 (ddd, 2H), 2.47-2.40 (m, 1H), 2.25-2.07 (m, 2H), 1.98-1.81 (m, 1H). LCMS (Method M): 467.1 [M+H]⁺.

### Example 316: (3S)—N-((1S)-1-(5-((5-chloro-4-fluoro-2,3-dihydro-1H-inden-2-yl)amino)pyridin-2-yl)-2,2,2-trifluoroethyl)-N-methyl-5-oxopyrrolidine-3-carboxamide—Isomer 1

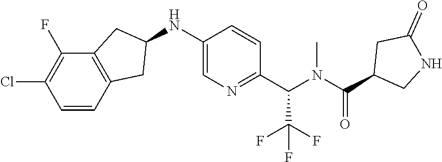 [SMILES: CN(C(=O)[C@@H]1CNC(=O)C1)[C@@H](c1ccc(N[C@H]2Cc3ccc(Cl)c(F)c3C2)cn1)C(F)(F)F]

### Example 317: (3S)—N-((1S)-1-(5-((5-chloro-4-fluoro-2,3-dihydro-1H-inden-2-yl)amino)pyridin-2-yl)-2,2,2-trifluoroethyl)-N-methyl-5-oxopyrrolidine-3-carboxamide—Isomer 2

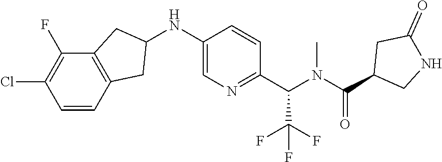 [SMILES: CN(C(=O)[C@@H]1CNC(=O)C1)[C@@H](c1ccc(NC2Cc3ccc(Cl)c(F)c3C2)cn1)C(F)(F)F]

The title compound was prepared in an analogous manner to Example 265 from Intermediate 202 (77 mg, 0.24 mmol), Intermediate 204 (90 mg, 0.49 mmol), AcOH (70 μL, 0.12 mmol) in MeOH (2.1 mL) at 50° C. for 16 h then sodium cyanoborohydride (61 mg, 0.98 mmol) at RT for 42 h. Purified by preparative HPLC (Kinetex XB-C18, 21×250 mm×5 μm, flow rate: 30 mL/min, 0.1% aqueous formic acid with MeCN 40% to 50% over 6 min, held for 5 min, ramped to 95% over 0.2 min and held for 1.8 min). Chiral purification was carried out by preparative SFC (Chiralpak®-IH, 30×250 mm×5 μm, temp 30° C., Back Pressure 100 bar, flow rate: 100 g/min, 60% CO₂ with 40% MeOH modifier) to provide Example 316 (13 mg) as Peak 1, Example 317 (12 mg) as Peak 2. Example 316: ¹H NMR (400 MHz; DMSO-d₆) δ: 8.02 (d, 1H), 7.65-7.62 (m, 1H), 7.37 (dd, 1H), 7.17-7.01 (m, 2H), 6.58-6.53 (m, 1H), 6.41-5.98 (m, 1H), 4.38-4.33 (m, 1H), 3.73-3.70 (m, 1H), 3.55 (dd, 1H), 3.43-3.37 (m, 2H), 3.31-3.24 (m, 2H), 2.96 (s, 3H), 2.88-2.66 (m, 2H), 2.49-2.32 (m, 2H). LCMS (Method A): 485.4 [M+H]⁺. Example 317: ¹H NMR (400 MHz; DMSO-d₆) δ: 8.02 (d, 1H), 7.65-7.62 (m, 1H), 7.39-7.24 (m, 1H), 7.17-7.01 (m, 2H), 6.58-6.53 (m, 1H), 6.42-5.98 (m, 1H), 4.34-4.33 (m, 1H), 3.75-3.70 (m, 1H), 3.56 (dd, 1H), 3.44-3.35 (m, 2H), 3.29-3.24 (m, 2H), 2.96 (s, 3H), 2.87-2.82 (m, 2H), 2.50-2.29 (m, 2H). LCMS (Method A): 485.3 [M+H]⁺.

### Example 318: (3S)—N-((1S)-1-(5-((4,5-Dichloro-2,3-dihydro-1H-inden-2-yl)amino)pyridin-2-yl)-2,2,2-trifluoroethyl)-N-methyl-5-oxopyrrolidine-3-carboxamide—Isomer 1

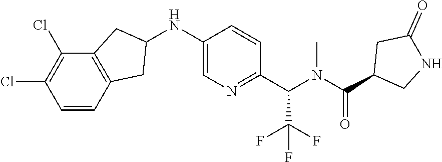 [SMILES: CN(C(=O)[C@@H]1CNC(=O)C1)[C@@H](c1ccc(NC2Cc3ccc(Cl)c(Cl)c3C2)cn1)C(F)(F)F]

### Example 319: (3S)—N-((1S)-1-(5-((4,5-Dichloro-2,3-dihydro-1H-inden-2-yl)amino)pyridin-2-yl)-2,2,2-trifluoroethyl)-N-methyl-5-oxopyrrolidine-3-carboxamide—Isomer 2

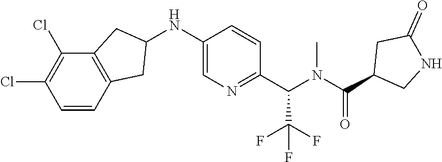 [SMILES: CN(C(=O)[C@@H]1CNC(=O)C1)[C@@H](c1ccc(NC2Cc3ccc(Cl)c(Cl)c3C2)cn1)C(F)(F)F]

The title compound was prepared in an analogous manner to Example 265 from Intermediate 202 (50 mg, 0.14 mmol), 4,5-dichloro-1,3-dihydro-2H-inden-2-one (57 mg, 0.28 mmol, CAS 69392-70-7), AcOH (41 μL, 0.07 mmol) in MeOH (1.4 mL) at 50° C. for 16 h then sodium cyanoborohydride (36 mg, 0.57 mmol) at RT for 16 h. Purified by preparative HPLC (Kinetex XB-C18, 21×250 mm×5 μm, flow rate: 30 mL/min, 0.1% aqueous formic acid with MeCN 40% to 60% over 6 min, held for 5 min, ramped to 95% over 0.2 min and held for 1.8 min). Chiral purification was carried out by preparative SFC (Chiralpak®-IH, 30×250 mm×5 μm, temp 30° C., Back Pressure 100 bar, flow rate: 100 g/min, 60% CO₂ with 40% MeOH modifier) to provide Example 318 (22 mg) as Peak 1, Example 319 (21 mg) as Peak 2. Example 318: ¹H NMR (400 MHz; DMSO-d₆) δ: 8.02-8.01 (m, 1H), 7.66-7.63 (m, 1H), 7.44 (d, 1H), 7.25 (d, 1H), 7.16 (d, 1H), 7.07-7.01 (m, 1H), 6.58-6.54 (m, 1H), 6.42-5.99 (m, 1H), 4.35-4.32 (m, 1H), 3.95-3.74 (m, 1H), 3.56 (dd, 1H), 3.46-3.36 (m, 2H), 3.27-3.24 (m, 1H), 2.96 (s, 3H), 2.92-2.85 (m, 2H), 2.49-2.33 (m, 2H). LCMS (Method A): 501.3 [M+H]⁺. Example 319: ¹H NMR (400 MHz; DMSO-d₆) δ: 8.02 (d, 1H), 7.66-7.63 (m, 1H), 7.44 (d, 1H), 7.25 (d, 1H), 7.16 (d, 1H), 7.08-7.01 (d, 1H), 6.58-6.54 (m, 1H), 6.38 (dd, 1H), 4.34-4.33 (m, 1H), 3.95-3.69 (m, 1H), 3.71 (dd, 1H), 3.50-3.25 (m, 3H), 2.96 (s, 3H), 2.92-2.85 (m, 2H), 2.43-2.35 (m, 2H). LCMS (Method A): 501.3 [M+H]⁺.

### Example 320: (3S)—N-((1S)-1-(5-((4,5-Dichloro-2,3-dihydro-1H-inden-2-yl)amino)pyridin-2-yl)-2,2,2-trifluoroethyl)-N-methyl-6-oxopiperidine-3-carboxamide—Isomer 1

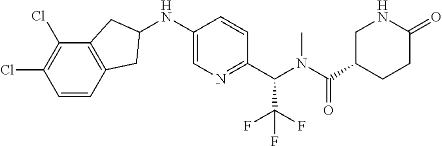 [SMILES: CN(C(=O)[C@H]1CCC(=O)NC1)[C@@H](c1ccc(NC2Cc3ccc(Cl)c(Cl)c3C2)cn1)C(F)(F)F]

### Example 321: ((3S)—N-((1S)-1-(5-((4,5-Dichloro-2,3-dihydro-1H-inden-2-yl)amino)pyridin-2-yl)-2,2,2-trifluoroethyl)-N-methyl-6-oxopiperidine-3-carboxamide—Isomer 2

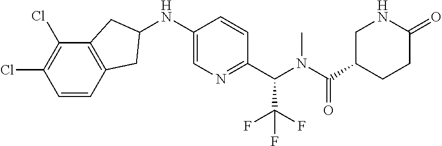 [SMILES: CN(C(=O)[C@H]1CCC(=O)NC1)[C@@H](c1ccc(NC2Cc3ccc(Cl)c(Cl)c3C2)cn1)C(F)(F)F]

The title compound was prepared in an analogous manner to Example 265 from Intermediate 206 (0.22 g, 0.61 mmol), 4,5-dichloro-1,3-dihydro-2H-inden-2-one (0.27 g, 1.21 mmol, CAS 69392-70-7), AcOH (18 mg, 0.30 mmol) in MeOH (3.0 mL) at 50° C. for 16 h then sodium cyanoborohydride (0.23 g, 3.63 mmol) at RT for 16 h. Purified by preparative HPLC (Gemini NX-C18, 21×250 mm×5 μm, flow rate: 30 mL/min, 0.05% aqueous ammonia with MeCN 45% to 50% over 6 min, held for 7 min, ramped to 95% over 0.2 min and held for 2.8 min). Chiral purification was carried out by preparative SFC (Chiralpak®-AD-H, 30×250 mm×5 μm, temp 30° C., Back Pressure 100 bar, flow rate: 100 g/min, 60% CO₂ with 40% MeOH modifier) to provide Example 320 (22 mg) as Peak 1, Example 321 (21 mg) as Peak 2. Example 320: ¹H NMR (400 MHz; DMSO-d₆) δ: 8.04-8.01 (m, 1H), 7.48 (s, 1H), 7.44 (d, 1H), 7.28-7.24 (m, 1H), 7.13 (d, 1H), 7.08-7.01 (m, 1H), 6.58-5.54 (m, 1H), 6.41-6.10 (m, 1H), 4.33 (dd, 1H), 3.46-3.32 (m, 2H), 3.23-3.17 (m, 3H), 3.02 (s, 3H), 2.99-2.71 (m, 2H), 2.24-2.19 (m, 2H), 1.85-1.76 (m, 2H). LCMS (Method A): 515.3 [M+H]⁺. Example 321: ¹H NMR (400 MHz; DMSO-d₆) δ: 8.02-8.01 (m, 1H), 7.48 (s, 1H), 7.44 (d, 1H), 7.28-7.24 (m, 1H), 7.13 (d, 1H), 7.04-7.02 (m, 1H), 6.58-6.54 (m, 1H), 6.41-6.10 (m, 1H), 4.33 (dd, 1H), 3.45-3.32 (m, 2H), 3.23-3.17 (m, 3H), 3.02 (s, 3H), 2.99-2.80 (m, 2H), 2.24-2.20 (m, 2H), 1.83-1.75 (m, 2H). LCMS (Method A): 515.3 [M+H]⁺.

### Example 322: N-((1S)-1-(5-((4,5-dichloro-2,3-dihydro-1H-inden-2-yl)amino)pyridin-2-yl)-2,2,2-trifluoroethyl)-N-methyl-3-(methylsulfonamido)bicyclo[1.1.1]pentane-1-carboxamide—Isomer 1

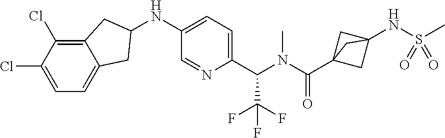 [SMILES: CN(C(=O)C12CC(NS(C)(=O)=O)(C1)C2)[C@@H](c1ccc(NC2Cc3ccc(Cl)c(Cl)c3C2)cn1)C(F)(F)F]

### Example 323: N-((1S)-1-(5-((4,5-dichloro-2,3-dihydro-1H-inden-2-yl)amino)pyridin-2-yl)-2,2,2-trifluoroethyl)-N-methyl-3-(methylsulfonamido)bicyclo[1.1.1]pentane-1-carboxamide—Isomer 2

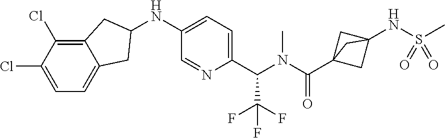 [SMILES: CN(C(=O)C12CC(NS(C)(=O)=O)(C1)C2)[C@@H](c1ccc(NC2Cc3ccc(Cl)c(Cl)c3C2)cn1)C(F)(F)F]

The title compound was prepared in an analogous manner to Example 265 from Intermediate 210 (0.26 g, 0.61 mmol), 4,5-dichloro-1,3-dihydro-2H-inden-2-one (0.24 g, 1.21 mmol, CAS 69392-70-7), AcOH (18 mg, 0.30 mmol) in MeOH (6.0 mL) at 50° C. for 16 h then sodium cyanoborohydride (0.15 g, 2.42 mmol) at RT for 16 h. Purified by preparative HPLC (Kinetex XB-C18, 21×250 mm×5 μm, flow rate: 30 mL/min, 0.1% aqueous formic acid with MeCN 25% to 70% over 10 min, held for 1.5 min, ramped to 95% over 0.2 min and held for 2.3 min). Chiral purification was carried out by preparative SFC (Chiralpak®-AD-H, 30×250 mm×5 μm, temp 30° C., Back Pressure 100 bar, flow rate: 100 g/min, 60% CO₂ with 40% MeOH modifier) to provide Example 322 (30 mg) as Peak 1, Example 323 (29 mg) as Peak 2. Example 322: ¹H NMR (400 MHz; DMSO-d₆) δ: 8.11 (br s, 1H), 8.03-8.01 (m, 1H), 7.44 (d, 1H), 7.27-7.20 (m, 1H), 7.15 (d, 1H), 7.09-7.01 (m, 1H), 6.60-6.56 (m, 1H), 6.37-5.76 (m, 1H), 4.35-4.32 (m, 1H), 3.46-3.36 (m, 2H), 3.03 (s, 3H), 2.96-2.84 (m, 5H), 2.40-2.07 (m, 6H). LCMS (Method A): 577.2 [M+H]⁺. Example 323: ¹H NMR (400 MHz; DMSO-d₆) δ: 8.12 (br s, 1H), 8.03-8.01 (m, 1H), 7.44 (d, 1H), 7.26-7.20 (m, 1H), 7.15 (d, 1H), 7.04-7.00 (m, 1H), 6.60-6.56 (m, 1H), 6.36-5.76 (m, 1H), 4.34-4.32 (m, 1H), 3.46-3.36 (m, 2H), 3.00 (s, 3H), 2.96-2.84 (m, 5H), 2.50-2.29 (m, 6H). LCMS (Method A): 577.2 [M+H]⁺.

### Example 324: N-((1S)-1-(5-((5-(Difluoromethoxy)-2,3-dihydro-1H-inden-2-yl)amino)pyridin-2-yl)-2,2,2-trifluoroethyl)-N-methyltetrahydro-2H-thiopyran-4-carboxamide 1,1-dioxide

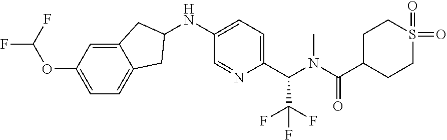 [SMILES: CN(C(=O)C1CCS(=O)(=O)CC1)[C@@H](c1ccc(NC2Cc3ccc(OC(F)F)cc3C2)cn1)C(F)(F)F]

The title compound (82 mg) was prepared in an analogous manner to Example 265 from Intermediate 166 (0.15 g, 0.41 mmol), Intermediate 211 (0.18 g, 0.82 mmol), AcOH (0.12 mL, 0.20 mmol) in MeOH (4.5 mL) at 50° C. for 16 h then sodium cyanoborohydride (0.10 g, 1.64 mmol) at RT for 16 h. Purified by preparative HPLC (Kinetex XB-C18, 21×250 mm×5 μm, flow rate: 30 mL/min, 0.1% aqueous formic acid with MeCN 50% isocratic over 9 min, ramped to 95% over 0.2 min and held for 1.8 min). ¹H NMR (400 MHz; 363 K, DMSO-d₆) δ: 8.05 (d, 1H), 7.26 (d, 1H), 7.20-7.09 (m, 1H), 7.07-7.02 (m, 3H), 6.96 (dd, 1H), 6.40-6.36 (m, 1H), 6.23 (d, 1H), 4.37-4.27 (m, 1H), 3.44-3.27 (m, 2H), 3.28-3.09 (m, 5H), 2.89-2.78 (m, 2H), 2.51 (s, 3H), 2.21-1.95 (m, 4H). LCMS (Method M): 548.2 [M+H]⁺.

### Example 325: N-((1S)-1-(5-((4,5-Dichloro-2,3-dihydro-1H-inden-2-yl)amino)pyridin-2-yl)-2,2,2-trifluoroethyl)-N-methyl-1-(methylsulfonyl)piperidine-4-carboxamide—Isomer 1

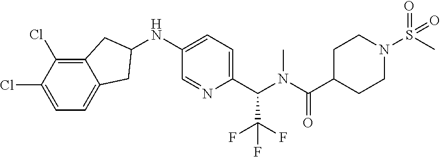 [SMILES: CN(C(=O)C1CCN(S(C)(=O)=O)CC1)[C@@H](c1ccc(NC2Cc3ccc(Cl)c(Cl)c3C2)cn1)C(F)(F)F]

### Example 326: N-((1S)-1-(5-((4,5-Dichloro-2,3-dihydro-1H-inden-2-yl)amino)pyridin-2-yl)-2,2,2-trifluoroethyl)-N-methyl-1-(methylsulfonyl)piperidine-4-carboxamide—Isomer 2

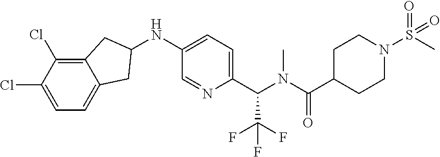 [SMILES: CN(C(=O)C1CCN(S(C)(=O)=O)CC1)[C@@H](c1ccc(NC2Cc3ccc(Cl)c(Cl)c3C2)cn1)C(F)(F)F]

The title compound was prepared in an analogous manner to Example 265 from Intermediate 213 (0.15 g, 0.41 mmol), 4,5-dichloro-1,3-dihydro-2H-inden-2-one (0.18 g, 0.82 mmol, CAS 69392-70-7), AcOH (0.12 mL, 0.20 mmol) in MeOH (4.5 mL) at 50° C. for 16 h then sodium cyanoborohydride (0.10 g, 1.64 mmol) at RT for 16 h. Purified by preparative HPLC (Kinetex XB-C18, 21×250 mm×5 μm, flow rate: 30 mL/min, 0.1% aqueous formic acid with MeCN 50% isocratic over 9 min, ramped to 95% over 0.2 min and held for 1.8 min). Chiral purification was carried out by preparative SFC (Lux Cellulose-4, 30×250 mm×5 μm, temp 30° C., Back Pressure 100 bar, flow rate: 100 g/min, 55% CO₂ with 45% MeOH modifier) to provide Example 325 (9 mg) as Peak 1, Example 326 (9 mg) as Peak 2. Example 325: ¹H NMR (400 MHz; DMSO-d₆) δ: 8.04-8.01 (m, 1H), 7.44 (d, 1H), 7.25 (d, 1H), 7.14 (d, 1H), 7.08-7.01 (m, 1H), 6.58-6.53 (m, 1H), 6.44-6.06 (m, 1H), 4.40-4.29 (m, 1H), 3.60-3.57 (m, 2H), 3.57-3.37 (m, 2H), 3.02 (s, 3H), 2.90-2.68 (m, 8H), 1.86-1.70 (m, 2H), 1.64-1.54 (m, 2H). LCMS (Method A): 579.2 [M+H]⁺. Example 326: ¹H NMR (400 MHz; DMSO-d₆) δ: 8.04-8.01 (m, 1H), 7.44 (d, 1H), 7.25 (d, 1H), 7.14 (d, 1H), 7.08-7.01 (m, 1H), 6.58-6.54 (m, 1H), 6.44-6.07 (m, 1H), 4.40-4.30 (m, 1H), 3.60-3.56 (m, 2H), 3.46-3.37 (m, 2H), 3.02 (s, 3H), 2.93-2.66 (m, 8H), 1.86-1.74 (m, 2H), 1.62-1.55 (m, 2H). LCMS (Method A): 579.2 [M+H]⁺.

### Example 327: (3S)—N-((1S)-1-(5-((5-Chloro-4-cyclopropyl-2,3-dihydro-1H-inden-2-yl)amino)pyridin-2-yl)-2,2,2-trifluoroethyl)-N-methyl-5-oxopyrrolidine-3-carboxamide—Isomer 1

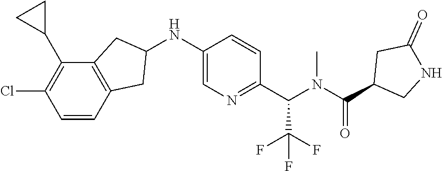 [SMILES: CN(C(=O)[C@@H]1CNC(=O)C1)[C@@H](c1ccc(NC2Cc3ccc(Cl)c(C4CC4)c3C2)cn1)C(F)(F)F]

### Example 328: (3S)—N-((1S)-1-(5-((5-Chloro-4-cyclopropyl-2,3-dihydro-1H-inden-2-yl)amino)pyridin-2-yl)-2,2,2-trifluoroethyl)-N-methyl-5-oxopyrrolidine-3-carboxamide—Isomer 2

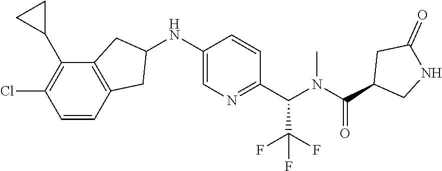 [SMILES: CN(C(=O)[C@@H]1CNC(=O)C1)[C@@H](c1ccc(NC2Cc3ccc(Cl)c(C4CC4)c3C2)cn1)C(F)(F)F]

The title compound was prepared in an analogous manner to Example 265 from Intermediate 202 (0.18 g, 0.51 mmol), Intermediate 193 (0.22 g, 1.02 mmol), AcOH (15 mg, 0.26 mmol) in MeOH (2.5 mL) at 50° C. for 16 h then sodium cyanoborohydride (0.19 g, 3.07 mmol) at RT for 16 h. Purified by preparative HPLC (Gemini NX-C18, 21×250 mm×5 μm, flow rate: 30 mL/min, 0.1% aqueous formic acid with MeCN 52% isocratic over 7.3 min, ramped to 95% over 0.2 min and held for 1.3 min). Chiral purification was carried out by preparative SFC (Chiralpak®-IH-H, 30×250 mm×5 μm, temp 30° C., Back Pressure 100 bar, flow rate: 100 g/min, 50% CO₂ with 50% MeOH modifier) to provide Example 327 (9 mg) as Peak 1, Example 328 (10 mg) as Peak 2. Example 327: ¹H NMR (400 MHz; DMSO-d₆) δ: 8.04-8.01 (m, 1H), 7.66-7.36 (m, 1H), 7.25-7.19 (m, 1H), 7.17-7.10 (m, 2H), 7.04-7.01 (m, 1H), 6.49-6.47 (m, 1H), 6.39-5.97 (m, 1H), 4.32-4.20 (m, 1H), 4.00-3.70 (m, 1H), 3.59-3.56 (m, 1H), 3.44 (dd, 1H), 3.28-3.21 (m, 2H), 2.96-2.74 (m, 5H), 2.43-2.32 (m, 2H), 1.82-1.81 (m, 1H), 0.98-0.95 (m, 2H), 0.65-0.61 (m, 2H). LCMS (Method A): 507.4 [M+H]⁺. Example 328: ¹H NMR (400 MHz; DMSO-d₆) δ: 8.02 (d, 1H), 7.66-7.63 (m, 1H), 7.26-7.01 (m, 4H), 6.51-6.47 (m, 1H), 6.41-5.97 (m, 1H), 4.26-4.23 (m, 1H), 3.74-3.72 (m, 1H), 3.59-3.55 (dd, 1H), 3.44 (dd, 1H), 3.29-3.21 (m, 2H), 2.97-2.74 (m, 5H), 2.43-2.33 (m, 2H), 1.83-1.79 (m, 1H), 0.98-0.95 (m, 2H), 0.65-0.61 (m, 2H). LCMS (Method A): 507.4 [M+H]⁺.

### Example 329: (3S)—N-((1S)-1-(5-((5-chloro-4-cyclopropyl-2,3-dihydro-1H-inden-2-yl)amino)pyridin-2-yl)-2,2,2-trifluoroethyl)-N-methyl-6-oxopiperidine-3-carboxamide—Isomer 1

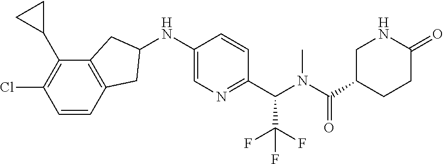 [SMILES: CN(C(=O)[C@H]1CCC(=O)NC1)[C@@H](c1ccc(NC2Cc3ccc(Cl)c(C4CC4)c3C2)cn1)C(F)(F)F]

### Example 330: (3S)—N-((1S)-1-(5-((5-chloro-4-cyclopropyl-2,3-dihydro-1H-inden-2-yl)amino)pyridin-2-yl)-2,2,2-trifluoroethyl)-N-methyl-6-oxopiperidine-3-carboxamide—Isomer 2

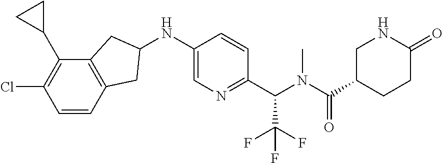 [SMILES: CN(C(=O)[C@H]1CCC(=O)NC1)[C@@H](c1ccc(NC2Cc3ccc(Cl)c(C4CC4)c3C2)cn1)C(F)(F)F]

The title compound was prepared in an analogous manner to Example 265 from Intermediate 206 (0.13 g, 0.34 mmol), Intermediate 193 (0.15 g, 0.69 mmol), AcOH (11 mg, 0.17 mmol) in MeOH (4.3 mL) at 50° C. for 3 h then sodium cyanoborohydride (85 mg, 1.35 mmol) at RT for 16 h. Purified by preparative HPLC (Gemini NX-C18, 21×250 mm×5 μm, flow rate: 30 mL/min, 0.1% aqueous formic acid with MeCN 40% to 47% over 6 min, held for 5 min, ramped to 95% over 0.2 min and held for 1.3 min). Chiral purification was carried out by preparative SFC (Chiralpak®-AD-H, 30×250 mm×5 μm, temp 30° C., Back Pressure 100 bar, flow rate: 100 g/min, 60% CO₂ with 40% (MeOH/MeCN 1:1) modifier) to provide Example 329 (16 mg) as Peak 1, Example 330 (17 mg) as Peak 2. Example 329: ¹H NMR (400 MHz; DMSO-d₆) δ: 8.01 (d, 1H), 7.48 (s, 1H), 7.27-7.01 (m, 4H), 6.48-6.46 (m, 1H), 6.41-6.11 (m, 1H), 4.30-4.19 (m, 1H), 3.44 (dd, 1H), 3.25-3.17 (m, 4H), 3.03 (s, 3H), 2.90 (dd 1H), 2.76 (dd, 1H), 2.27-2.19 (m, 2H), 1.83-1.77 (m, 3H), 0.96 (dd, 2H), 0.63 (dd, 2H). LCMS (Method A): 521.4 [M+H]⁺. Example 330: ¹H NMR (400 MHz; DMSO-d₆) δ: 8.02 (d, 1H), 7.49 (s, 1H), 7.49-7.01 (m, 4H), 6.50-6.46 (m, 1H), 6.15-6.10 (m, 1H), 4.30-4.20 (m, 1H), 3.44 (dd, 1H), 3.27-3.17 (m, 4H), 3.03 (s, 3H), 2.90 (dd, 1H), 2.74 (dd 1H), 2.24-2.19 (m, 2H), 1.83-1.78 (m, 3H), 0.98-0.95 (m, 2H), 0.65-0.61 (m, 2H). LCMS (Method A): 521.4 [M+H]⁺.

### Example 331: N-((1S)-1-(6-((5-Chloro-4-fluoro-2,3-dihydro-1H-inden-2-yl)amino)pyridin-3-yl)-2,2,2-trifluoroethyl)-N-methyltetrahydro-2H-thiopyran-4-carboxamide 1,1-dioxide—Isomer 1

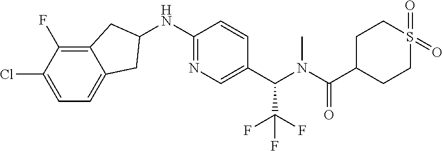 [SMILES: CN(C(=O)C1CCS(=O)(=O)CC1)[C@@H](c1ccc(NC2Cc3ccc(Cl)c(F)c3C2)nc1)C(F)(F)F]

### Example 332: N-((1S)-1-(6-((5-Chloro-4-fluoro-2,3-dihydro-1H-inden-2-yl)amino)pyridin-3-yl)-2,2,2-trifluoroethyl)-N-methyltetrahydro-2H-thiopyran-4-carboxamide 1,1-dioxide—Isomer 2

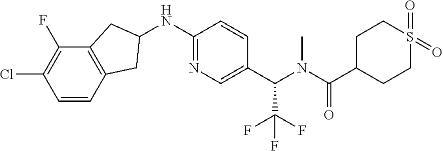 [SMILES: CN(C(=O)C1CCS(=O)(=O)CC1)[C@@H](c1ccc(NC2Cc3ccc(Cl)c(F)c3C2)nc1)C(F)(F)F]

The title compound was prepared in an analogous manner to Intermediate 3 from Intermediate 216 (0.15 g, 0.32 mmol), pyridine (0.3 mL), tetrahydro-2H-thiopyran-4-carboxylic acid 1,1-dioxide (0.34 g, 1.91 mmol, CAS 64096-87-3), T3P® (0.95 mL, 3.18 mmol) at RT for 16 h. Purified by flash chromatography (silica gel, eluting 10-50% EtOAc in hexane). Chiral purification was carried out by preparative SFC (Chiralpak®-IH, 30×250 mm×5 μm, temp 30° C., Back Pressure 100 bar, flow rate: 100 g/min, 75% CO₂ with 25% MeOH modifier) to provide Example 331 (9 mg) as Peak 1, Example 332 (10 mg) as Peak 2. Example 331: ¹H NMR (400 MHz; DMSO-d₆) δ: 8.11-7.99 (m, 1H), 7.49-7.34 (m, 2H), 7.30-7.22 (m, 1H), 7.12 (d, 1H), 6.58-6.52 (m, 1H), 6.41-6.03 (m, 1H), 4.68 (dd, 1H), 3.28-3.08 (m, 7H), 2.98-2.84 (m, 5H), 2.11-1.94 (m, 4H). LCMS (Method O): 534.2 [M+H]⁺. Example 332: ¹H NMR (400 MHz; DMSO-d₆) δ: 8.11-8.00 (m, 1H), 7.49-7.34 (m, 2H), 7.30-7.23 (m, 1H), 7.11 (d, 1H), 6.58-6.52 (m, 1H), 6.41-6.03 (m, 1H), 4.67 (dd, 1H), 3.28-3.09 (m, 7H), 2.93-2.83 (m, 5H), 2.11-1.94 (m, 4H). LCMS (Method O): 534.2 [M+H]⁺.

### Example 333: N-((1S)-1-(5-((4-(Difluoromethoxy)-2,3-dihydro-1H-inden-2-yl)amino)pyridin-2-yl)-2,2,2-trifluoroethyl)-N-methyltetrahydro-2H-thiopyran-4-carboxamide 1,1-dioxide

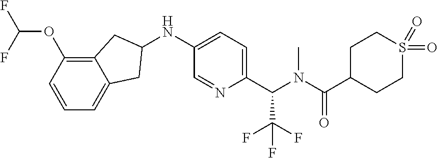 [SMILES: CN(C(=O)C1CCS(=O)(=O)CC1)[C@@H](c1ccc(NC2Cc3cccc(OC(F)F)c3C2)cn1)C(F)(F)F]

The title compound (0.14 g) was prepared in an analogous manner to Example 265 from Intermediate 166 (0.20 g, 0.55 mmol), Intermediate 218 (0.16 g, 0.82 mmol), AcOH (0.12 mL, 0.20 mmol) in MeOH (4.1 mL) at 50° C. for 3 h then sodium cyanoborohydride (0.10 g, 1.63 mmol) at RT for 16 h. Purified by preparative HPLC (Gemini NX-C18, 21×250 mm×5 μm, flow rate: 30 mL/min, 0.1% aqueous formic acid with MeCN 40% to 48% over 6 min, held for 5 min, ramped to 95% over 0.2 min and held for 1.3 min). ¹H NMR (400 MHz; 353 K, DMSO-d₆) δ: 8.06 (d, 1H), 7.29-7.03 (m, 5H), 6.99 (dd, 1H), 6.46-6.31 (m, 1H), 6.24 (d, 1H), 4.39-4.29 (m, 1H), 3.46-3.30 (m, 2H), 3.30-3.04 (m, 8H), 2.97-2.79 (m, 2H), 2.19-1.92 (m, 4H). LCMS (Method M): 548.3 [M+H]⁺.

### Example 334: N³-((1S)-1-(5-((4,5-Dichloro-2,3-dihydro-1H-inden-2-yl)amino)pyridin-2-yl)-2,2,2-trifluoroethyl)-N¹,N³-dimethylazetidine-1,3-dicarboxamide—Isomer 1

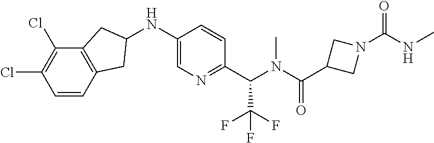 [SMILES: CNC(=O)N1CC(C(=O)N(C)[C@@H](c2ccc(NC3Cc4ccc(Cl)c(Cl)c4C3)cn2)C(F)(F)F)C1]

### Example 335: N³-((1S)-1-(5-((4,5-Dichloro-2,3-dihydro-1H-inden-2-yl)amino)pyridin-2-yl)-2,2,2-trifluoroethyl)-N¹,N³-dimethylazetidine-1,3-dicarboxamide—Isomer 2

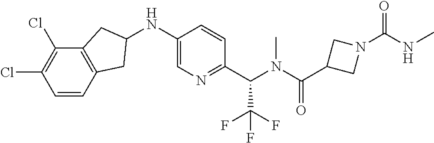 [SMILES: CNC(=O)N1CC(C(=O)N(C)[C@@H](c2ccc(NC3Cc4ccc(Cl)c(Cl)c4C3)cn2)C(F)(F)F)C1]

The title compound was prepared in an analogous manner to Example 265 from Intermediate 222 (0.12 g, 0.33 mmol), 4,5-dichloro-1,3-dihydro-2H-inden-2-one (0.15 g, 0.67 mmol, CAS 69392-70-7), AcOH (11 mg, 0.17 mmol) in MeOH (3.7 mL) at 50° C. for 2 h then sodium cyanoborohydride (0.11 g, 1.67 mmol) at RT for 2 h. Purified by preparative HPLC (Gemini NX-C18, 21×250 mm×5 μm, flow rate: 30 mL/min, 0.1% aqueous formic acid with MeCN 40% to 55% over 4 min, held for 3 min, ramped to 95% over 0.2 min and held for 1.3 min). Chiral purification was carried out by preparative SFC (Chiralpak®-IH-H, 30×250 mm×5 μm, temp 30° C., Back Pressure 100 bar, flow rate: 100 g/min, 65% CO₂ with 35% MeOH modifier) to provide Example 334 (16 mg) as Peak 1, Example 335 (17 mg) as Peak 2. Example 334: ¹H NMR (400 MHz; DMSO-d₆) δ: 8.01 (d, 1H), 7.44 (d, 1H), 7.26-7.16 (m, 2H), 7.08-7.01 (m, 1H), 6.58-6.54 (m, 1H), 6.40-5.65 (m, 2H), 4.40-4.30 (m, 1H), 3.87-3.75 (m, 5H), 3.47-3.36 (m, 2H), 2.92-2.81 (m, 5H), 2.53 (s, 3H). LCMS (Method A): 530.3 [M+H]⁺. Example 335: ¹H NMR (400 MHz; DMSO-d₆) δ: 8.01 (d, 1H), 7.43 (d, 1H), 7.26-7.16 (m, 2H), 7.06-7.01 (m, 1H), 6.56 (d, 1H), 6.44-5.65 (m, 2H), 4.44 (br s, 1H), 3.84-3.74 (m, 5H), 3.45-3.36 (m, 2H), 2.93-2.75 (m, 5H), 2.52 (s, 3H). LCMS (Method A): 530.3 [M+H]⁺.

### Example 336: (3S)—N-((1S)-1-(5-((5-chloro-4-fluoro-2,3-dihydro-1H-inden-2-yl)amino)pyridin-2-yl)-2,2,2-trifluoroethyl)-N-methyl-6-oxopiperidine-3-carboxamide—Isomer 1

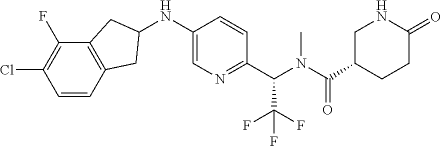 [SMILES: CN(C(=O)[C@H]1CCC(=O)NC1)[C@@H](c1ccc(NC2Cc3ccc(Cl)c(F)c3C2)cn1)C(F)(F)F]

### Example 337: (3S)—N-((1S)-1-(5-((5-chloro-4-fluoro-2,3-dihydro-1H-inden-2-yl)amino)pyridin-2-yl)-2,2,2-trifluoroethyl)-N-methyl-6-oxopiperidine-3-carboxamide—Isomer 2

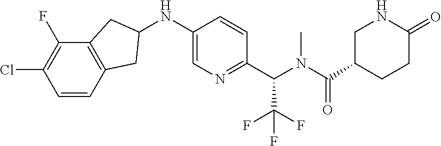 [SMILES: CN(C(=O)[C@H]1CCC(=O)NC1)[C@@H](c1ccc(NC2Cc3ccc(Cl)c(F)c3C2)cn1)C(F)(F)F]

The title compound was prepared in an analogous manner to Example 265 from Intermediate 206 (0.20 g, 0.48 mmol), Intermediate 204 (0.18 g, 0.97 mmol), AcOH (15 mg, 0.24 mmol) in MeOH (4.0 mL) at 50° C. for 16 h then sodium cyanoborohydride (0.12 g, 1.94 mmol) at RT for 42 h. Purified by preparative HPLC (Gemini NX-C18, 21×250 mm×5 μm, flow rate: 30 mL/min, 0.1% aqueous formic acid with MeCN 40% to 45% over 6 min, held for 5 min, ramped to 95% over 0.2 min and held for 1.3 min). Chiral purification was carried out by preparative SFC (Chiralpak®-AD-H, 30×250 mm×5 μm, temp 30° C., Back Pressure 100 bar, flow rate: 100 g/min, 63% CO₂ with 37% (IPA/MeCN 1:1) modifier) to provide Example 336 (19 mg) as Peak 1, Example 337 (24 mg) as Peak 2. Example 336: ¹H NMR (400 MHz; DMSO-d₆) δ: 8.02 (s, 1H), 7.47 (s, 1H), 7.39-7.35 (m, 1H), 7.28-7.02 (m, 3H), 6.53 (d, 1H), 6.39-6.14 (m, 1H), 4.43-4.28 (m, 1H), 3.44-3.35 (m, 2H), 3.25-3.17 (m, 3H), 3.02 (s, 3H), 2.87-2.67 (m, 2H), 2.24-2.20 (m, 2H), 1.83-1.76 (m, 2H). LCMS (Method A): 499.3 [M+H]⁺. Example 337: ¹H NMR (400 MHz; DMSO-d₆) δ: 8.02 (s, 1H), 7.48 (s, 1H), 7.39-7.35 (m, 1H), 7.27-7.02 (m, 3H), 6.57-6.53 (m, 1H), 6.41-6.14 (m, 1H), 4.41-4.29 (m, 1H), 3.44-3.35 (m, 2H), 3.25-3.17 (m, 3H), 3.02 (s, 3H), 2.87-2.83 (m, 2H), 2.24-2.20 (m, 2H), 1.82-1.76 (m, 2H). LCMS (Method A): 499.3 [M+H]⁺.

### Example 338: (3R)—N-((1S)-1-(6-((5-Chloro-4-fluoro-2,3-dihydro-1H-inden-2-yl)amino)pyridin-3-yl)-2,2,2-trifluoroethyl)-N-methyl-5-oxopyrrolidine-3-carboxamide—Isomer 1

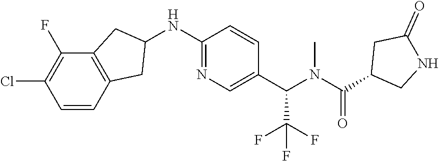 [SMILES: CN(C(=O)[C@H]1CNC(=O)C1)[C@@H](c1ccc(NC2Cc3ccc(Cl)c(F)c3C2)nc1)C(F)(F)F]

### Example 339: (3R)—N-((1S)-1-(6-((5-Chloro-4-fluoro-2,3-dihydro-1H-inden-2-yl)amino)pyridin-3-yl)-2,2,2-trifluoroethyl)-N-methyl-5-oxopyrrolidine-3-carboxamide—Isomer 2

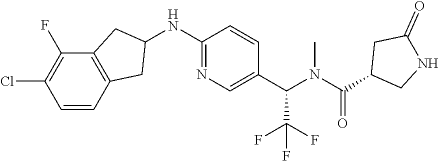 [SMILES: CN(C(=O)[C@H]1CNC(=O)C1)[C@@H](c1ccc(NC2Cc3ccc(Cl)c(F)c3C2)nc1)C(F)(F)F]

The title compound was prepared in an analogous manner to Intermediate 3 from Intermediate 216 (50 mg, 0.11 mmol), pyridine (0.1 mL), (3R)-5-oxopyrrolidine-3-carboxylic acid (36 mg, 0.28 mmol, CAS 428518-37-0), T3P® (0.67 mL, 1.12 mmol) at RT for 16 h. Purified by flash chromatography (silica gel, eluting 5-10% MeOH in DCM). Chiral purification was carried out by preparative SFC (Lux-Cellulose-4, 30×250 mm×5 μm, temp 30° C., Back Pressure 100 bar, flow rate: 100 g/min, 73% CO₂ with 27% MeOH modifier) to provide Example 338 (7 mg) as Peak 1, Example 339 (7 mg) as Peak 2. Example 338: ¹H NMR (400 MHz; DMSO-d₆) δ: 8.09-8.00 (m, 1H), 7.69-7.66 (m, 1H), 7.48-7.34 (m, 2H), 7.29-7.26 (m, 1H), 7.11 (d, 1H), 6.58-6.52 (m, 1H), 6.41-6.02 (m, 1H), 4.69-4.65 (m, 1H), 4.00-3.66 (m, 1H), 3.48-3.43 (m, 1H), 3.38-3.27 (m, 2H), 2.89-2.83 (m, 5H), 2.72-2.65 (m, 2H), 2.30-2.24 (m, 1H). LCMS (Method O): 485.2 [M+H]⁺. Example 339: ¹H NMR (400 MHz; DMSO-d₆) δ: 8.09-8.01 (m, 1H), 7.65 (s, 1H), 7.48-7.34 (m, 2H), 7.25-7.23 (m, 1H), 7.11 (d, 1H), 6.58-6.52 (m, 1H), 6.40-6.02 (m, 1H), 4.68-4.66 (m, 1H), 3.98-3.68 (m, 3H), 3.48-3.38 (m, 2H), 2.89-2.83 (m, 5H), 2.33-2.24 (m, 1H). LCMS (Method O): 485.2 [M+H]⁺.

### Example 340: N-((1S)-1-(5-((4,5-Dichloro-2,3-dihydro-1H-inden-2-yl)amino)pyridin-2-yl)-2,2,2-trifluoroethyl)-N-methyl-6-oxo-5-azaspiro[3.4]octane-2-carboxamide—Isomer 1

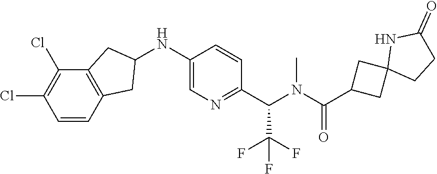 [SMILES: CN(C(=O)C1CC2(CCC(=O)N2)C1)[C@@H](c1ccc(NC2Cc3ccc(Cl)c(Cl)c3C2)cn1)C(F)(F)F]

### Example 341: N-((1S)-1-(5-((4,5-Dichloro-2,3-dihydro-1H-inden-2-yl)amino)pyridin-2-yl)-2,2,2-trifluoroethyl)-N-methyl-6-oxo-5-azaspiro[3.4]octane-2-carboxamide—Isomer 2

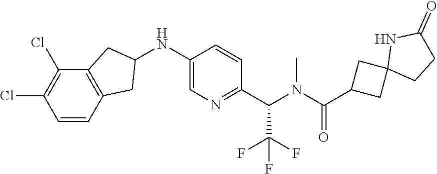 [SMILES: CN(C(=O)C1CC2(CCC(=O)N2)C1)[C@@H](c1ccc(NC2Cc3ccc(Cl)c(Cl)c3C2)cn1)C(F)(F)F]

### Example 342: N-((1S)-1-(5-((4,5-Dichloro-2,3-dihydro-1H-inden-2-yl)amino)pyridin-2-yl)-2,2,2-trifluoroethyl)-N-methyl-6-oxo-5-azaspiro[3.4]octane-2-carboxamide—Isomer 3

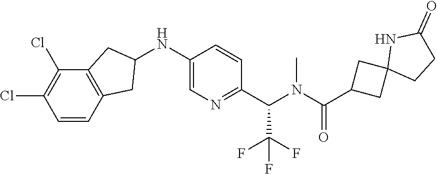 [SMILES: CN(C(=O)C1CC2(CCC(=O)N2)C1)[C@@H](c1ccc(NC2Cc3ccc(Cl)c(Cl)c3C2)cn1)C(F)(F)F]

### Example 343: N-((1S)-1-(5-((4,5-Dichloro-2,3-dihydro-1H-inden-2-yl)amino)pyridin-2-yl)-2,2,2-trifluoroethyl)-N-methyl-6-oxo-5-azaspiro[3.4]octane-2-carboxamide—Isomer 4

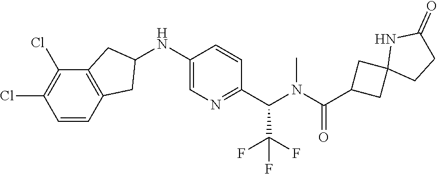 [SMILES: CN(C(=O)C1CC2(CCC(=O)N2)C1)[C@@H](c1ccc(NC2Cc3ccc(Cl)c(Cl)c3C2)cn1)C(F)(F)F]

The title compound was prepared in an analogous manner to Example 265 from Intermediate 224 (0.23 g, 0.57 mmol), 4,5-dichloro-1,3-dihydro-2H-inden-2-one (0.23 g, 1.14 mmol, CAS 69392-70-7), AcOH (17 mg, 0.28 mmol) in MeOH (5.7 mL) at 50° C. for 20 h then sodium cyanoborohydride (0.14 g, 2.28 mmol) at RT for 16 h. Purified by preparative HPLC (Gemini NX-C18, 21×250 mm×5 μm, flow rate: 30 mL/min, 0.1% aqueous formic acid with MeCN 40% to 48% over 6 min, held for 5 min, ramped to 95% over 0.2 min and held for 1.8 min). Chiral purification was carried out by preparative SFC (Chiralpak®-IH, 30×250 mm×5 μm, temp 30° C., Back Pressure 100 bar, flow rate: 100 g/min, 60% CO₂ with 40% MeOH modifier) to provide Example 340 (20 mg) as Peak 1, Example 341 (18 mg) as Peak 2, Example 342 (6 mg) as Peak 3, Example 343 (8 mg) as Peak 4. Example 340: ¹H NMR (400 MHz; DMSO-d₆) δ: 8.03-8.01 (m, 1H), 7.97 (s, 1H), 7.44 (d, 1H), 7.26-7.20 (m, 1H), 7.13 (d, 1H), 7.07-7.00 (m, 1H), 6.55 (d, 1H), 6.35-5.76 (m, 1H), 4.34-4.32 (m, 1H), 3.46-3.36 (m, 2H), 3.19-3.14 (m, 1H), 2.92-2.84 (m, 5H), 2.36-2.23 (m, 3H), 2.21-2.16 (m, 5H). LCMS (Method A): 541.3 [M+H]⁺. Example 341: ¹H NMR (400 MHz; DMSO-d₆) δ: 8.02-8.00 (m, 1H), 7.96 (s, 1H), 7.44 (d, 1H), 7.26-7.20 (m, 1H), 7.13 (d, 1H), 7.07-7.00 (m, 1H), 6.54 (d, 1H), 6.39-5.75 (m, 1H), 4.34-4.32 (m, 1H), 3.46-3.36 (m, 2H), 3.21-3.12 (m, 1H), 2.92-2.72 (m, 5H), 2.40-2.32 (m, 3H), 2.31-2.19 (m, 5H). LCMS (Method A): 541.3 [M+H]⁺. Example 342: ¹H NMR (400 MHz; DMSO-d₆) δ: 8.17-8.13 (m, 1H), 8.01-8.00 (m, 1H), 7.44 (d, 1H), 7.31-7.21 (m, 1H), 7.14 (d, 1H), 7.06-7.01 (m, 1H), 6.60-6.54 (m, 1H), 6.43-5.51 (m, 1H), 4.34-4.32 (m, 1H), 3.57-3.36 (m, 3H), 2.92-2.75 (m, 5H), 2.45-2.42 (m, 2H), 2.36-2.34 (m, 2H), 2.12-2.01 (m, 2H), 2.00-1.96 (m, 2H). LCMS (Method H): 541.3 [M+H]⁺. Example 343: ¹H NMR (400 MHz; DMSO-d₆) δ: 8.16-8.13 (m, 1H), 8.02-8.00 (m, 1H), 7.44 (d, 1H), 7.26-7.21 (m, 1H), 7.14 (d, 1H), 7.06-7.01 (m, 1H), 6.59-6.54 (m, 1H), 6.43-5.76 (m, 1H), 4.34-4.32 (m, 1H), 3.46-3.38 (m, 3H), 2.91-2.82 (m, 5H), 2.50-2.45 (m, 2H), 2.37-2.19 (m, 2H), 2.12-1.98 (m, 4H). LCMS (Method A): 541.3 [M+H]⁺.

### Example 344: N-((1S)-1-(6-((4,5-Dichloro-2,3-dihydro-1H-inden-2-yl)amino)pyridin-3-yl)-2,2,2-trifluoroethyl)-N-methyltetrahydro-2H-thiopyran-4-carboxamide 1,1-dioxide—Isomer 1

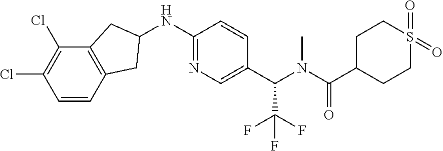 [SMILES: CN(C(=O)C1CCS(=O)(=O)CC1)[C@@H](c1ccc(NC2Cc3ccc(Cl)c(Cl)c3C2)nc1)C(F)(F)F]

### Example 345: N-((1S)-1-(6-((4,5-Dichloro-2,3-dihydro-1H-inden-2-yl)amino)pyridin-3-yl)-2,2,2-trifluoroethyl)-N-methyltetrahydro-2H-thiopyran-4-carboxamide 1,1-dioxide—Isomer 2

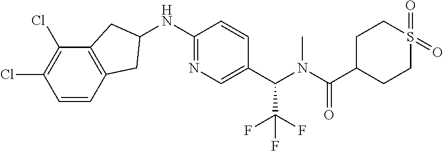 [SMILES: CN(C(=O)C1CCS(=O)(=O)CC1)[C@@H](c1ccc(NC2Cc3ccc(Cl)c(Cl)c3C2)nc1)C(F)(F)F]

The title compound was prepared in an analogous manner to Intermediate 3 from Intermediate 226 (60 mg, 0.11 mmol), pyridine (0.2 mL), tetrahydro-2H-thiopyran-4-carboxylic acid 1,1-dioxide (79 mg, 0.44 mmol, CAS 64096-87-3), T3P® (0.35 g, 0.44 mmol) at RT for 8 h. Purified by flash chromatography (silica gel, eluting 0-60% EtOAc in petroleum ether). Chiral purification was carried out by preparative SFC (Chiralpak®-AD-H, 30×250 mm×5 μm, temp 30° C., Back Pressure 100 bar, flow rate: 100 g/min, 50% CO₂ with 50% MeOH modifier) to provide Example 344 (9 mg) as Peak 1, Example 345 (10 mg) as Peak 2. Example 344: ¹H NMR (400 MHz; DMSO-d₆) δ: 8.11-8.00 (m, 1H), 7.49-7.39 (m, 2H), 7.31-7.23 (m, 2H), 6.58-6.53 (m, 1H), 6.41-6.03 (m, 1H), 4.66-4.62 (m, 1H), 3.41-3.34 (m, 2H), 3.25-3.09 (m, 5H), 2.95-2.67 (m, 5H), 2.11-1.94 (m, 4H). LCMS (Method O): 550.2 [M+H]⁺. Example 345: ¹H NMR (400 MHz; DMSO-d₆) δ: 8.11-8.00 (m, 1H), 7.49-7.39 (m, 2H), 7.31-7.13 (m, 2H), 6.58-6.52 (m, 1H), 6.41-6.03 (m, 1H), 4.66-4.62 (m, 1H), 3.41-3.31 (m, 2H), 3.22-3.08 (m, 5H), 2.92-2.87 (m, 5H), 2.11-1.97 (m, 4H). LCMS (Method O): 550.2 [M+H]⁺.

### Example 346: (3R)—N-((1S)-1-(6-((4,5-Dichloro-2,3-dihydro-1H-inden-2-yl)amino)pyridin-3-yl)-2,2,2-trifluoroethyl)-N-methyl-5-oxopyrrolidine-3-carboxamide—Isomer 1

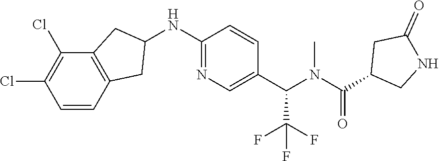 [SMILES: CN(C(=O)[C@H]1CNC(=O)C1)[C@@H](c1ccc(NC2Cc3ccc(Cl)c(Cl)c3C2)nc1)C(F)(F)F]

### Example 347: (3R)—N-((1S)-1-(6-((4,5-Dichloro-2,3-dihydro-1H-inden-2-yl)amino)pyridin-3-yl)-2,2,2-trifluoroethyl)-N-methyl-5-oxopyrrolidine-3-carboxamide—Isomer 2

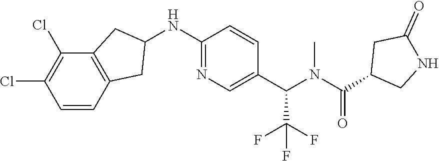 [SMILES: CN(C(=O)[C@H]1CNC(=O)C1)[C@@H](c1ccc(NC2Cc3ccc(Cl)c(Cl)c3C2)nc1)C(F)(F)F]

The title compound was prepared in an analogous manner to Intermediate 3 from Intermediate 226 (50 mg, 0.09 mmol), pyridine (0.1 mL), (3R)-5-oxopyrrolidine-3-carboxylic acid (48 mg, 0.37 mmol, CAS 428518-37-0), T3P® (50% in EtOAc, 0.56 mL, 0.92 mmol) at RT for 16 h. Purified by flash chromatography (silica gel, eluting 0-100% EtOAc in petroleum ether). Chiral purification was carried out by preparative SFC (Chiralpak®-IH, 30×250 mm×5 μm, temp 30° C., Back Pressure 100 bar, flow rate: 100 g/min, 55% CO₂ with 45% (MeOH/MeCN 1:1) modifier) to provide Example 346 (13 mg) as Peak 1, Example 347 (13 mg) as Peak 2. Example 346: ¹H NMR (400 MHz; DMSO-d₆) δ: 8.10-8.01 (m, 1H), 7.69-7.66 (m, 1H), 7.48-7.42 (m, 2H), 7.29-7.23 (m, 2H), 6.58-6.52 (m, 1H), 6.41-6.02 (m, 1H), 4.64 (dd, 1H), 4.00-3.68 (m, 1H), 3.48-3.36 (m, 4H), 2.94-2.67 (m, 5H), 2.50-2.46 (m, 1H), 2.33-2.23 (m, 1H). LCMS (Method A): 501.3 [M+H]⁺. Example 347: ¹H NMR (400 MHz; DMSO-d₆) δ: 8.10-8.01 (m, 1H), 7.69-7.66 (m, 1H), 7.48-7.40 (m, 2H), 7.29-7.23 (m, 2H), 6.58-6.53 (d, 1H), 6.41-6.02 (m, 1H), 4.66-4.62 (m, 1H), 4.00-3.68 (m, 1H), 3.48-3.28 (m, 4H), 2.94-2.70 (m, 5H), 2.50-2.46 (m, 1H), 2.33-2.23 (m, 1H). LCMS (Method A): 501.3 [M+H]⁺.

### Example 348: N-((1S)-1-(5-((5-Chloro-4-fluoro-2,3-dihydro-1H-inden-2-yl)amino)pyridin-2-yl)-2,2,2-trifluoroethyl)-N,2-dimethylpyrimidine-5-carboxamide

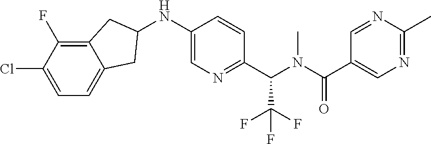 [SMILES: Cc1ncc(C(=O)N(C)[C@@H](c2ccc(NC3Cc4ccc(Cl)c(F)c4C3)cn2)C(F)(F)F)cn1]

The title compound (19 mg) was prepared in an analogous manner to Intermediate 3 from Intermediate 162 (0.15 g, 0.35 mmol), pyridine (1.5 mL), 2-methylpyrimidine-5-carboxylic acid (72 mg, 0.52 mmol, CAS 5194-32-1), T3P® (50% in EtOAc, 1.10 g, 3.45 mmol) at RT for 16 h. Purified by flash chromatography (silica gel, eluting 0-100% EtOAc in petroleum ether). Chiral purification was carried out by preparative SFC (Lux-i-Amylose-3, 30×250 mm×5 μm, temp 30° C., Back Pressure 100 bar, flow rate: 100 g/min, 53% CO₂ with 47% (MeOH/MeCN 1:1) modifier) to provide the title compound as Peak 1. ¹H NMR (400 MHz; DMSO-d₆) δ: 8.84-8.00 (m, 2H), 8.05 (s, 1H), 7.39-7.29 (m, 2H), 7.13 (d, 1H), 7.07 (dd, 1H), 6.60 (d, 1H), 6.48-5.61 (m, 1H), 4.38-4.36 (m, 1H), 3.45-3.36 (m, 2H), 2.97 (s, 3H), 2.88-2.84 (m, 2H), 2.68 (s, 3H). LCMS (Method A): 494.3 [M+H]⁺.

### Example 349: 2-Cyano-N-((1S)-1-(5-((4,5-dichloro-2,3-dihydro-1H-inden-2-yl)amino)pyridin-2-yl)-2,2,2-trifluoroethyl)-N-methylacetamide—Isomer 1

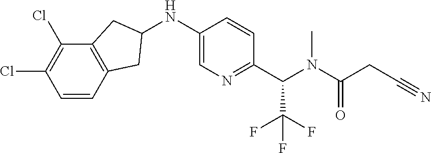 [SMILES: CN(C(=O)CC#N)[C@@H](c1ccc(NC2Cc3ccc(Cl)c(Cl)c3C2)cn1)C(F)(F)F]

### Example 350: 2-Cyano-N-((1S)-1-(5-((4,5-dichloro-2,3-dihydro-1H-inden-2-yl)amino)pyridin-2-yl)-2,2,2-trifluoroethyl)-N-methylacetamide—Isomer 2

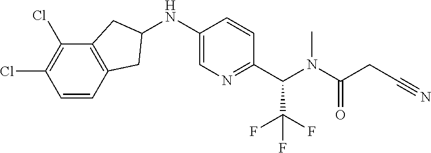 [SMILES: CN(C(=O)CC#N)[C@@H](c1ccc(NC2Cc3ccc(Cl)c(Cl)c3C2)cn1)C(F)(F)F]

To a stirred solution of 2-cyanoacetic acid (60 mg, 0.35 mmol, CAS 372-09-8), EDCI (67 mg, 0.70 mmol) and HOBt (47 mg, 0.35 mmol) in THF (1.0 mL) was added triethylamine (71 mg, 0.70 mmol) and Intermediate 228 (0.10 g, 0.23 mmol) and the mixture was stirred at RT for 16 h. The mixture was diluted with water, then extracted with EtOAc. The organic layer was dried over Na₂SO₄, filtered and concentrated under reduced pressure. The crude product was purified by flash chromatography (silica gel, eluting 0-70% EtOAc in hexane). Chiral purification was carried out by preparative SFC (Chiralpak®-IH, 30×250 mm×5 μm, temp 30° C., Back Pressure 100 bar, flow rate: 100 g/min, 65% CO₂ with 35% MeOH modifier) to provide Example 349 (21 mg) as Peak 1, Example 350 (23 mg) as Peak 2. Example 349: ¹H NMR (400 MHz; DMSO-d₆) δ: 8.02 (d, 1H), 7.44 (d, 1H), 7.35-7.19 (m, 2H), 7.07-7.01 (m, 1H), 6.58 (d, 1H), 6.31-5.78 (m, 1H), 4.35-4.16 (m, 3H), 3.46-3.37 (m, 2H), 2.92-2.74 (m, 5H). LCMS (Method H): 457.3 [M+H]⁺. Example 350: ¹H NMR (400 MHz; DMSO-de) δ: 8.02 (d, 1H), 7.44 (d, 1H), 7.35-7.19 (m, 2H), 7.07-7.01 (m, 1H), 6.59-6.56 (m, 1H), 6.32-5.78 (m, 1H), 4.35-4.16 (m, 3H), 3.46-3.37 (m, 2H), 2.92-2.73 (m, 5H). LCMS (Method H): 457.3 [M+H]⁺.

### Example 351: N-((1S)-1-(5-((4,5-Dichloro-2,3-dihydro-1H-inden-2-yl)amino)pyridin-2-yl)-2,2,2-trifluoroethyl)-N-methyl-1-(tetrahydrofuran-3-carbonyl)azetidine-3-carboxamide—Isomer 1

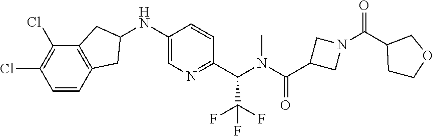 [SMILES: CN(C(=O)C1CN(C(=O)C2CCOC2)C1)[C@@H](c1ccc(NC2Cc3ccc(Cl)c(Cl)c3C2)cn1)C(F)(F)F]

### Example 352: N-((1S)-1-(5-((4,5-Dichloro-2,3-dihydro-1H-inden-2-yl)amino)pyridin-2-yl)-2,2,2-trifluoroethyl)-N-methyl-1-(tetrahydrofuran-3-carbonyl)azetidine-3-carboxamide—Isomer 2

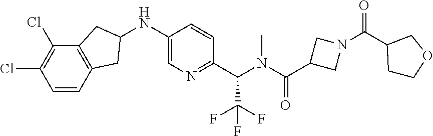 [SMILES: CN(C(=O)C1CN(C(=O)C2CCOC2)C1)[C@@H](c1ccc(NC2Cc3ccc(Cl)c(Cl)c3C2)cn1)C(F)(F)F]

### Example 353: N-((1S)-1-(5-((4,5-Dichloro-2,3-dihydro-1H-inden-2-yl)amino)pyridin-2-yl)-2,2,2-trifluoroethyl)-N-methyl-1-(tetrahydrofuran-3-carbonyl)azetidine-3-carboxamide—Isomer 3

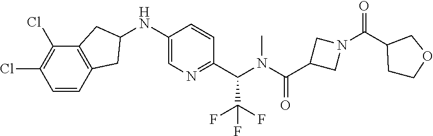 [SMILES: CN(C(=O)C1CN(C(=O)C2CCOC2)C1)[C@@H](c1ccc(NC2Cc3ccc(Cl)c(Cl)c3C2)cn1)C(F)(F)F]

### Example 354: N-((1S)-1-(5-((4,5-Dichloro-2,3-dihydro-1H-inden-2-yl)amino)pyridin-2-yl)-2,2,2-trifluoroethyl)-N-methyl-1-(tetrahydrofuran-3-carbonyl)azetidine-3-carboxamide—Isomer 4

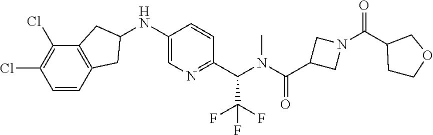 [SMILES: CN(C(=O)C1CN(C(=O)C2CCOC2)C1)[C@@H](c1ccc(NC2Cc3ccc(Cl)c(Cl)c3C2)cn1)C(F)(F)F]

The title compound was prepared in an analogous manner to Intermediate 3 from Intermediate 228 (0.20 g, 0.48 mmol), pyridine (0.1 mL), 1-(tetrahydrofuran-3-carbonyl)azetidine-3-carboxylic acid (0.46 g, 0.95 mmol, CAS 1344264-60-3), T3P® (50% in EtOAc, 2.85 mL, 4.77 mmol) at RT for 2 h. Chiral purification was carried out by preparative SFC (Chiralpak®-IH, 30×250 mm×5 μm, temp 30° C., Back Pressure 100 bar, flow rate: 100 g/min, 63% CO₂ with 37% MeOH modifier) to provide Example 351 (8 mg) as Peak 1, Example 352 (9 mg) as Peak 2, Example 353 (7 mg) as Peak 3, Example 354 (6 mg) as Peak 4. Example 351: ¹H NMR (400 MHz; DMSO-d₆) δ: 8.04-8.01 (m, 1H), 7.44 (d, 1H), 7.25 (d, 1H), 7.21-7.16 (m, 1H), 7.07-7.01 (m, 1H), 6.59-6.55 (m, 1H), 6.44-5.65 (m, 1H), 4.36-4.27 (m, 3H), 4.09-4.05 (m, 1H), 3.90-3.81 (m, 3H), 3.72-3.58 (m, 3H), 3.46-3.36 (m, 2H), 3.08-2.92 (m, 1H), 2.91-2.83 (m, 5H), 2.10-1.82 (m, 2H). LCMS (Method H): 571.3 [M+H]⁺. Example 352: ¹H NMR (400 MHz; DMSO-d₆) δ: 8.04-8.01 (m, 1H), 7.44 (d, 1H), 7.25 (d, 1H), 7.21-7.16 (m, 1H), 7.08-7.01 (m, 1H), 6.60-5.55 (m, 1H), 6.40-5.67 (m, 1H), 4.36-4.27 (m, 3H), 4.18-4.00 (m, 1H), 3.90-3.84 (m, 3H), 3.72-3.58 (m, 3H), 3.42-3.37 (m, 2H), 3.00 (ddd, 1H), 2.92-2.83 (m, 5H), 2.10-1.85 (m, 2H). LCMS (Method H): 571.3 [M+H]⁺. Example 353: ¹H NMR (400 MHz; DMSO-d₆) δ: 8.04-8.02 (m, 1H), 7.44 (d, 1H), 7.25 (d, 1H), 7.21-7.17 (m, 1H), 7.07-7.01 (m, 1H), 6.59-6.55 (m, 1H), 6.41-5.68 (m, 1H), 4.38-4.33 (m, 3H), 4.20-4.03 (m, 1H), 3.89-3.82 (m, 3H), 3.72-3.58 (m, 3H), 3.46-3.36 (m, 2H), 3.05-2.98 (m, 1H), 2.92-2.83 (m, 5H), 2.10-1.85 (m, 2H). LCMS (Method H): 571.3 [M+H]⁺. Example 354: ¹H NMR (400 MHz; DMSO-d₆) δ: 8.04-8.01 (m, 1H), 7.44 (d, 1H), 7.25 (d, 1H), 7.21-7.17 (m, 1H), 7.07-7.01 (m, 1H), 6.59-6.55 (m, 1H), 6.41-5.68 (m, 1H), 4.42-4.25 (m, 3H), 4.20-4.00 (m, 1H), 3.89-3.82 (m, 3H), 3.72-3.60 (m, 3H), 3.46-3.37 (m, 2H), 3.05-2.98 (m, 1H), 2.92-2.83 (m, 5H), 2.10-1.85 (m, 2H). LCMS (Method H): 571.3 [M+H]⁺.

### Example 355: N-((1S)-1-(5-((4,5-Dichloro-2,3-dihydro-1H-inden-2-yl)amino)pyridin-2-yl)-2,2,2-trifluoroethyl)-N-methylthietane-3-carboxamide 1,1-dioxide

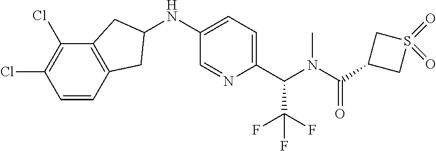

The title compound (22 mg) was prepared in an analogous manner to Example 265 from Intermediate 242 (80 mg, 0.24 mmol), 4,5-dichloro-1,3-dihydro-2H-inden-2-one (95 mg, 0.47 mmol, CAS 69392-70-7), AcOH (7 μL, 0.12 mmol) in MeOH (2.4 mL) at 50° C. for 6 h then sodium cyanoborohydride (60 mg, 0.95 mmol) at RT for 16 h. An additional portion of sodium cyanoborohydride (30 mg, 0.48 mmol) was added and stirred at RT for 90 h. Purified by preparative HPLC (Kinetex-XB-C18, 21×250 mm×5 μm, flow rate: 28 mL/min, 0.1% aqueous formic acid with MeCN 25% to 55% over 5 min, held for 0.5 min, to 75% over 3.5 min, ramped to 95% over 0.2 min and held for 2.3 min). ¹H NMR (400 MHz; DMSO-d₆) δ: 8.02 (d, 1H), 7.45 (d, 1H), 7.33-7.13 (m, 2H), 7.07-7.03 (m, 1H), 6.62-6.57 (m, 1H), 6.36 (dd, 1H), 4.63-4.20 (m, 5H), 4.00-3.60 (m, 1H), 3.49-3.36 (m, 2H), 2.95 (s, 3H), 2.93-2.83 (m, 2H). LCMS (Method M): 522.1 [M+H]⁺.

### Example 356: N-((1S)-1-(5-((5-Chloro-4-(methoxymethyl)-2,3-dihydro-1H-inden-2-yl)amino)pyridin-2-yl)-2,2,2-trifluoroethyl)-N-methyltetrahydro-2H-thiopyran-4-carboxamide 1,1-dioxide

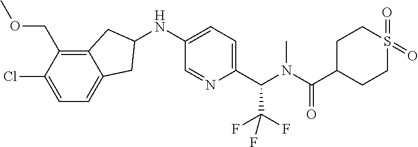 [SMILES: COCc1c(Cl)ccc2c1CC(Nc1ccc([C@H](N(C)C(=O)C3CCS(=O)(=O)CC3)C(F)(F)F)nc1)C2]

The title compound (15 mg) was prepared in an analogous manner to Intermediate 193 from Intermediate 229 (0.10 g, 0.17 mmol), potassium (methoxymethyl)trifluoroborate (51 mg, 0.34 mmol, CAS 910251-11-5), Pd(dppf)Cl₂ (12.3 mg, 0.02 mmol) and K₃PO₄ (0.11 g, 0.50 mmol) in toluene (1 mL) and water (0.2 mL) at 90° C. for 16 h. Purified by preparative HPLC (Gemini-NX C18, 21×250 mm×5 μm, flow rate: 30 mL/min, 0.1% aqueous formic acid with MeCN 48% isocratic over 11.5 min, ramped to 95% over 0.2 min and held for 1.8 min). ¹H NMR (400 MHz; DMSO-d₆) δ: 8.02 (d, 1H), 7.25 (dd, 2H), 7.15 (d, 1H), 7.08-7.00 (m, 1H), 6.42-6.23 (m, 1H), 4.54-4.45 (m, 2H), 4.29 (dd, 1H), 3.44 (dd, 2H), 3.27-3.10 (m, 8H), 3.02 (s, 3H), 2.90-2.75 (m, 2H), 2.16-2.02 (m, 5H). LCMS (Method M): 560.3 [M+H]⁺.

### Example 357: N-((1S)-1-(5-((4,5-Dichloro-2,3-dihydro-1H-inden-2-yl)amino)pyridin-2-yl)-2,2,2-trifluoroethyl)-N-methyltetrahydrothiophene-3-carboxamide 1,1-dioxide—Isomer 1

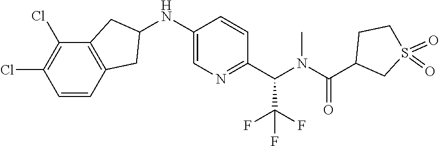 [SMILES: CN(C(=O)C1CCS(=O)(=O)C1)[C@@H](c1ccc(NC2Cc3ccc(Cl)c(Cl)c3C2)cn1)C(F)(F)F]

### Example 358: N-((1S)-1-(5-((4,5-Dichloro-2,3-dihydro-1H-inden-2-yl)amino)pyridin-2-yl)-2,2,2-trifluoroethyl)-N-methyltetrahydrothiophene-3-carboxamide 1,1-dioxide—Isomer 2

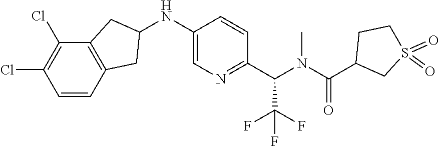 [SMILES: CN(C(=O)C1CCS(=O)(=O)C1)[C@@H](c1ccc(NC2Cc3ccc(Cl)c(Cl)c3C2)cn1)C(F)(F)F]

### Example 359: N-((1S)-1-(5-((4,5-Dichloro-2,3-dihydro-1H-inden-2-yl)amino)pyridin-2-yl)-2,2,2-trifluoroethyl)-N-methyltetrahydrothiophene-3-carboxamide 1,1-dioxide—Isomer 3

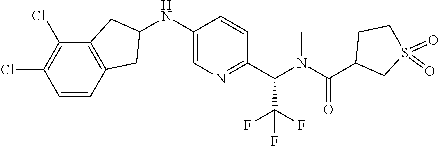 [SMILES: CN(C(=O)C1CCS(=O)(=O)C1)[C@@H](c1ccc(NC2Cc3ccc(Cl)c(Cl)c3C2)cn1)C(F)(F)F]

### Example 360: N-((1S)-1-(5-((4,5-Dichloro-2,3-dihydro-1H-inden-2-yl)amino)pyridin-2-yl)-2,2,2-trifluoroethyl)-N-methyltetrahydrothiophene-3-carboxamide 1,1-dioxide—Isomer 4

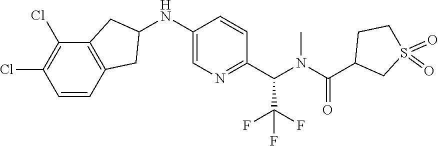 [SMILES: CN(C(=O)C1CCS(=O)(=O)C1)[C@@H](c1ccc(NC2Cc3ccc(Cl)c(Cl)c3C2)cn1)C(F)(F)F]

The title compound was prepared in an analogous manner to Intermediate 3 from Intermediate 228 (0.20 g, 0.47 mmol), pyridine (0.7 mL), tetrahydrothiophene-3-carboxylic acid 1,1-dioxide (0.15 g, 0.93 mmol, CAS 4785-67-5), T3P® (50% in EtOAc, 2.85 mL, 4.77 mmol) at RT for 6 h. The crude product was purified by flash chromatography (silica gel, eluting 0-100% EtOAc in petroleum ether). Chiral purification was carried out by preparative SFC (Chiralpak®-IH, 30×250 mm×5 μm, temp 30° C., Back Pressure 100 bar, flow rate: 100 g/min, 70% CO₂ with 30% MeOH modifier) to provide Example 357 (24 mg) as Peak 1, Example 358 (20 mg) as Peak 2, Example 359 (22 mg) as Peak 3, Example 360 (23 mg) as Peak 4. Example 357: ¹H NMR (400 MHz; DMSO-d₆) δ: 8.04-8.01 (m, 1H), 7.44 (d, 1H), 7.31-7.24 (m, 1H), 7.17 (d, 1H), 7.08-7.01 (m, 1H), 6.59-6.55 (m, 1H), 6.39-6.07 (m, 1H), 4.35-4.32 (m, 1H), 3.80 (dd, 1H), 3.48-3.36 (m, 3H), 3.23-3.09 (m, 3H), 3.02 (s, 3H), 2.93-2.75 (m, 2H), 2.38-2.35 (m, 1H), 2.08-2.06 (m, 1H). LCMS (Method H): 536.3 [M+H]⁺. Example 358: ¹H NMR (400 MHz; DMSO-d₆) δ: 8.04-8.01 (m, 1H), 7.44 (d, 1H), 7.31-7.24 (m, 1H), 7.17 (d, 1H), 7.08-7.01 (m, 1H), 6.57-6.55 (m, 1H), 6.39-6.07 (m, 1H), 4.34-4.32 (m, 1H), 3.80 (dd, 1H), 3.48-3.36 (m, 3H), 3.26-3.09 (m, 3H), 3.02 (s, 3H), 2.92-2.75 (m, 2H), 2.38-2.32 (m, 1H), 2.11-2.02 (m, 1H). LCMS (Method H): 536.3 [M+H]⁺. Example 359: ¹H NMR (400 MHz; DMSO-d₆) δ: 8.06-8.03 (m, 1H), 7.44 (d, 1H), 7.30-7.24 (m, 1H), 7.22 (d, 1H), 7.08-7.01 (m, 1H), 6.61-6.55 (m, 1H), 6.41-6.13 (m, 1H), 4.38-4.32 (m, 1H), 3.81 (dd, 1H), 3.46-3.35 (m, 3H), 3.24-3.11 (m, 3H), 3.03 (s, 3H), 2.92-2.73 (m, 2H), 2.50-2.44 (m, 1H), 2.07-2.03 (m, 1H). LCMS (Method H): 536.3 [M+H]⁺. Example 360: ¹H NMR (400 MHz; DMSO-d₆) δ: 8.05-8.01 (m, 1H), 7.44 (d, 1H), 7.30-7.24 (m, 1H), 7.17 (d, 1H), 7.08-7.01 (m, 1H), 6.61-6.55 (m, 1H), 6.41-6.11 (m, 1H), 4.35-4.31 (m, 1H), 3.81 (dd, 1H), 3.46-3.34 (m, 3H), 3.27-3.11 (m, 3H), 3.04 (s, 3H), 2.92-2.73 (m, 2H), 2.51-2.44 (m, 1H), 2.07-2.03 (m, 1H). LCMS (Method H): 536.2 [M+H]⁺.

### Example 361: N-((1S)-1-(5-((5-Chloro-4-fluoro-2,3-dihydro-1H-inden-2-yl)amino)pyridin-2-yl)-2,2,2-trifluoroethyl)-2-(1,1-dioxidoisothiazolidin-2-yl)-N-methylacetamide—Isomer 1

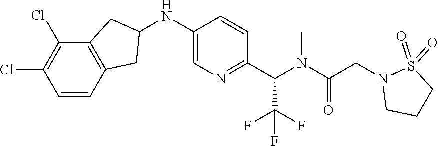 [SMILES: CN(C(=O)CN1CCCS1(=O)=O)[C@@H](c1ccc(NC2Cc3ccc(Cl)c(Cl)c3C2)cn1)C(F)(F)F]

### Example 362: N-((1S)-1-(5-((5-Chloro-4-fluoro-2,3-dihydro-1H-inden-2-yl)amino)pyridin-2-yl)-2,2,2-trifluoroethyl)-2-(1,1-dioxidoisothiazolidin-2-yl)-N-methylacetamide—Isomer 2

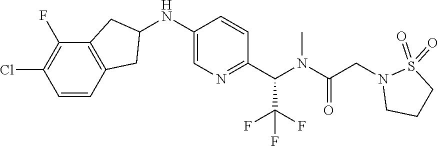 [SMILES: CN(C(=O)CN1CCCS1(=O)=O)[C@@H](c1ccc(NC2Cc3ccc(Cl)c(F)c3C2)cn1)C(F)(F)F]

The title compound was prepared in an analogous manner to Intermediate 3 from Intermediate 162 (0.15 g, 0.37 mmol), pyridine (0.5 mL), 2-isothiazolidineacetic acid, 1,1-dioxide (0.13 g, 0.73 mmol, CAS 63459-24-5), T3P® (50% in MeTHF, 2.23 mL, 3.65 mmol) at RT for 6 h. Purified by flash chromatography (silica gel, eluting 0-100% EtOAc in petroleum ether). Chiral purification was carried out by preparative SFC (Chiralpak®-IG, 30×250 mm×5 μm, temp 30° C., Back Pressure 100 bar, flow rate: 100 g/min, 65% CO₂ with 35% (IPA/MeCN 1:1) modifier) to provide Example 361 (31 mg) as Peak 1, Example 362 (33 mg) as Peak 2. Example 361: ¹H NMR (400 MHz; DMSO-d₆) δ: 8.01 (d, 1H), 7.37 (dd, 1H), 7.29-7.07 (m, 2H), 7.02 (dd, 1H), 6.55 (d, 1H), 6.34-5.88 (m, 1H), 4.36 (dd, 1H), 4.21-3.98 (m, 2H), 3.44-3.35 (m, 4H), 3.21 (dd, 2H), 2.96 (s, 3H), 2.86 (dd, 2H), 2.32-2.26 (m, 2H). LCMS (Method H): 535.3 [M+H]⁺. Example 362: ¹H NMR (400 MHz; DMSO-d₆) δ: 8.02 (d, 1H), 7.36 (dd, 1H), 7.17 (d, 1H), 7.12 (d, 1H), 7.03 (dd, 1H), 6.55 (d, 1H), 6.35-5.84 (m, 1H), 4.37-4.34 (m, 1H), 4.21-3.99 (m, 2H), 3.44-3.37 (m, 4H), 3.21 (dd, 2H), 2.96 (s, 3H), 2.87-2.73 (m, 2H), 2.32-2.24 (m, 2H). LCMS (Method H): 535.3 [M+H]⁺.

### Example 363: (1,3-trans)-N¹-((1S)-1-(5-((4,5-Dichloro-2,3-dihydro-1H-inden-2-yl)amino)pyridin-2-yl)-2,2,2-trifluoroethyl)-N¹,N³-dimethylcyclobutane-1,3-dicarboxamide

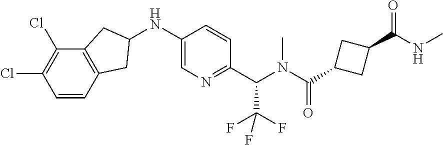 [SMILES: CNC(=O)[C@H]1C[C@H](C(=O)N(C)[C@@H](c2ccc(NC3Cc4ccc(Cl)c(Cl)c4C3)cn2)C(F)(F)F)C1]

To a stirred solution of Intermediate 233 (41 mg, 0.08 mmol) in DCM (1.2 mL) was added triethylamine (28 mg, 0.28 mmol), EDCI (18 mg, 0.10 mmol) and methylamine (2M in THF, 48 μL, 0.10 mmol) and the mixture was stirred at RT for 16 h. The mixture washed with 1M aqueous HCl dried over Na₂SO₄, filtered and concentrated under reduced pressure. The crude product was purified by preparative HPLC (Kinatex-XB C18, 21×250 mm×5 μm, flow rate: 30 mL/min, 0.1% aqueous formic acid with MeCN 35% isocratic for 6 min then to 40% over 5 min, ramped to 95% over 0.2 min and held for 1.8 min) to provide the title compound (2.4 mg). ¹H NMR (400 MHz; DMSO-d₆) δ: 8.01 (d, 1H), 7.70 (d, 1H), 7.44 (d, 1H), 7.26 (d, 1H), 7.15 (d, 1H), 7.03 (dd, 1H), 6.55 (d, 1H), 6.40 (dd, 1H), 4.40-4.28 (m, 1H), 3.49-3.35 (m, 3H), 2.99-2.83 (m, 3H), 2.81 (s, 3H), 2.57 (d, 3H), 2.45-2.21 (m, 4H). LCMS (Method M): 529.1 [M+H]⁺.

### Example 364: N-((1S)-1-(5-((5-Chloro-4-fluoro-2,3-dihydro-1H-inden-2-yl)amino)pyridin-2-yl)-2,2,2-trifluoroethyl)-N-methyl-1-(5-methyl-1,3,4-oxadiazol-2-yl)azetidine-3-carboxamide—Isomer 1

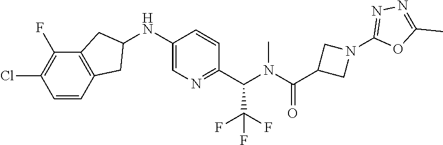 [SMILES: Cc1nnc(N2CC(C(=O)N(C)[C@@H](c3ccc(NC4Cc5ccc(Cl)c(F)c5C4)cn3)C(F)(F)F)C2)o1]

### Example 365: N-((1S)-1-(5-((5-Chloro-4-fluoro-2,3-dihydro-1H-inden-2-yl)amino)pyridin-2-yl)-2,2,2-trifluoroethyl)-N-methyl-1-(5-methyl-1,3,4-oxadiazol-2-yl)azetidine-3-carboxamide—Isomer 2

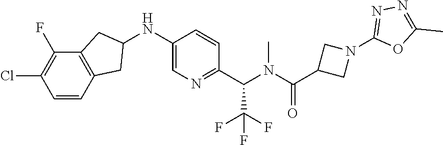 [SMILES: Cc1nnc(N2CC(C(=O)N(C)[C@@H](c3ccc(NC4Cc5ccc(Cl)c(F)c5C4)cn3)C(F)(F)F)C2)o1]

The title compound was prepared in an analogous manner to Intermediate 3 from Intermediate 162 (0.20 g, 0.44 mmol), triethylamine (0.19 mL, 1.33 mmol), 1-(5-methyl-1,3,4-oxadiazol-2-yl)azetidine-3-carboxylic acid (0.17 g, 0.89 mmol, CAS 2303330-69-8), T3P® (50% in MeTHF, 1.31 mL, 4.44 mmol) at RT for 16 h. Purified by flash chromatography (silica gel, eluting 0-100% EtOAc in hexane). Chiral purification was carried out by preparative SFC (Chiralpak®-AD-H, 30×250 mm×5 μm, temp 30° C., Back Pressure 100 bar, flow rate: 100 g/min, 60% CO₂ with 40% MeOH modifier) to provide Example 364 (21 mg) as Peak 2, Example 365 (22 mg) as Peak 1. Example 364: ¹H NMR (400 MHz; DMSO-d₆) δ: 8.04-8.01 (m, 1H), 7.37 (dd, 1H), 7.35-7.17 (m, 1H), 7.12 (d, 1H), 7.03 (dd, 1H), 6.59-6.54 (m, 1H), 6.40-5.65 (m, 1H), 4.23-4.25 (m, 3H), 4.23-4.06 (m, 3H), 3.44-3.35 (m, 2H), 2.87-2.75 (m, 5H), 2.33 (s, 3H). LCMS (Method O): 539.3 [M+H]⁺. Example 365: ¹H NMR (400 MHz; DMSO-d₆) δ: 8.04-7.97 (m, 1H), 7.39 (dd, 1H), 7.27-7.17 (m, 1H), 7.12 (d, 1H), 7.03 (dd, 1H), 6.59-6.54 (m, 1H), 6.46-5.66 (m, 1H), 4.41-4.22 (m, 3H), 4.19-4.06 (m, 3H), 3.44-3.35 (m, 2H), 2.96-2.75 (m, 5H), 2.33 (s, 3H). LCMS (Method 0): 539.3 [M+H]⁺.

### Example 366: N-((1S)-1-(5-((5-chloro-4-fluoro-2,3-dihydro-1H-inden-2-yl)amino)pyridin-2-yl)-2,2,2-trifluoroethyl)-2-hydroxy-N-methylacetamide—Isomer 1

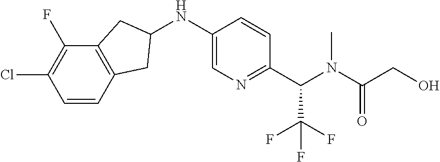 [SMILES: CN(C(=O)CO)[C@@H](c1ccc(NC2Cc3ccc(Cl)c(F)c3C2)cn1)C(F)(F)F]

### Example 367: N-((1S)-1-(5-((5-chloro-4-fluoro-2,3-dihydro-1H-inden-2-yl)amino)pyridin-2-yl)-2,2,2-trifluoroethyl)-2-hydroxy-N-methylacetamide—Isomer 2

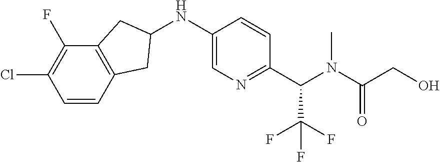 [SMILES: CN(C(=O)CO)[C@@H](c1ccc(NC2Cc3ccc(Cl)c(F)c3C2)cn1)C(F)(F)F]

To a stirred solution of Intermediate 234 (0.15 g, 0.26 mmol) in DCM (0.1 mL) was added TFA (0.04 mL) at RT for 4 h. The mixture was concentrated under reduced pressure then diluted with aqueous NaHCO₃ and extracted into EtOAc. The combined organics were dried over Na₂SO₄, filtered and concentrated under reduced pressure. The crude was purified by flash chromatography (silica gel, eluting 0-100% EtOAc in petroleum ether). Chiral purification was carried out by preparative SFC (Lux-Cellulose-4, 30×250 mm×5 μm, temp 30° C., Back Pressure 100 bar, flow rate: 100 g/min, 90% CO₂ with 10% MeOH modifier) to provide Example 366 (12 mg) as Peak 2, Example 367 (16 mg) as Peak 1. Example 366: ¹H NMR (400 MHz; DMSO-d₆) δ: 8.02 (d, 1H), 7.37 (dd, 1H), 7.28-7.11 (m, 2H), 7.06-7.01 (m, 1H), 6.55 (d, 1H), 6.39-5.82 (m, 1H), 5.10-4.79 (m, 1H), 4.38-4.19 (m, 3H), 3.44-3.35 (m, 2H), 2.86-2.74 (m, 5H). LCMS (Method H): 432.3 [M+H]⁺. Example 367: ¹H NMR (400 MHz; DMSO-d₆) δ: 8.02 (s, 1H), 7.37 (dd, 1H), 7.28-7.11 (m, 2H), 7.06-7.01 (m, 1H), 6.55 (d, 1H), 6.39-5.82 (m, 1H), 5.11-4.49 (m, 1H), 4.39-4.19 (m, 3H), 3.44-3.33 (m, 2H), 2.86-2.74 (m, 5H). LCMS (Method H): 432.3 [M+H]⁺.

### Example 368: N-((1S)-1-(5-((5-Chloro-4-fluoro-2,3-dihydro-1H-inden-2-yl)amino)pyridin-2-yl)-2,2,2-trifluoroethyl)-N,N′-dimethylcyclopropane-1,1-dicarboxamide

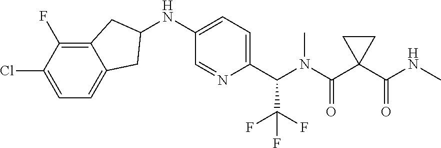 [SMILES: CNC(=O)C1(C(=O)N(C)[C@@H](c2ccc(NC3Cc4ccc(Cl)c(F)c4C3)cn2)C(F)(F)F)CC1]

The title compound (63 mg) was prepared in an analogous manner to Example 265 from Intermediate 236 (0.23 g, 0.70 mmol), Intermediate 204 (0.26 g, 1.39 mmol), AcOH (0.21 g, 0.35 mmol) in MeOH (4.6 mL) at 50° C. for 17 h then sodium cyanoborohydride (0.22 g, 3.49 mmol) at RT for 2 h. Purified by preparative HPLC (Kinetex-XB C18, 21×250 mm×5 μm, flow rate: 30 mL/min, 0.1% aqueous formic acid with MeCN 58% isocratic over 11 min, ramped to 95% over 0.2 min and held for 1.8 min). ¹H NMR (400 MHz; DMSO-d₆) δ: 8.03 (d, 1H), 7.63 (d, 1H), 7.38 (dd, 1H), 7.24 (d, 1H), 7.13 (d, 1H), 7.06 (dd, 1H), 6.55 (d, 1H), 6.30 (dd, 1H), 4.42-4.33 (m, 1H), 3.46-3.33 (m, 2H), 2.96-2.83 (m, 5H), 2.62 (d, 3H), 1.40-1.33 (m, 1H), 1.29-1.22 (m, 1H), 1.18-1.06 (m, 2H). LCMS (Method M): 499.2 [M+H]⁺.

### Example 369: N-((1S)-1-(5-((5-Chloro-4-isopropyl-2,3-dihydro-1H-inden-2-yl)amino)pyridin-2-yl)-2,2,2-trifluoroethyl)-N-methyltetrahydro-2H-thiopyran-4-carboxamide 1,1-dioxide

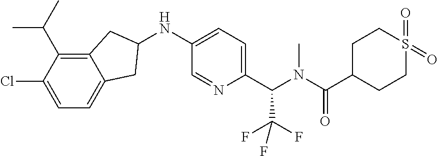 [SMILES: CC(C)c1c(Cl)ccc2c1CC(Nc1ccc([C@H](N(C)C(=O)C3CCS(=O)(=O)CC3)C(F)(F)F)nc1)C2]

The title compound (4.4 mg) was prepared in an analogous manner to Intermediate 193 from Intermediate 229 (60 mg, 0.10 mmol), isopropylboronic acid (18 mg, 0.20 mmol), Pd(dppf)Cl₂·DCM (8.3 mg, 0.01 mmol) and K₃PO₄ (64 mg, 0.30 mmol) in toluene (0.9 mL) and water (0.2 mL) at 90° C. for 16 h. Purified by preparative HPLC (Gemini-NX C18, 21×250 mm×5 μm, flow rate: 30 mL/min, 0.1% aqueous formic acid with MeCN 63% isocratic over 11 min, ramped to 95% over 0.2 min and held for 1.8 min). ¹H NMR (400 MHz; DMSO-d₆) δ: 8.02 (d, 1H), 7.29-6.99 (m, 4H), 6.49 (d, 1H), 6.38 (dd, 1H), 4.34-4.22 (m, 1H), 3.29-3.07 (m, 7H), 3.01 (s, 3H), 2.85-2.74 (m, 2H), 2.62 (d, 2H), 2.14-1.91 (m, 4H), 1.52 (dd, 2H), 0.93 (dd, 3H). LCMS (Method M): 558.1 [M+H]⁺.

### Example 370: N-((1S)-1-(5-((5-Chloro-4-fluoro-2,3-dihydro-1H-inden-2-yl)amino)pyridin-2-yl)-2,2,2-trifluoroethyl)-N-methyl-1-(oxetane-3-carbonyl)azetidine-3-carboxamide—Isomer 1

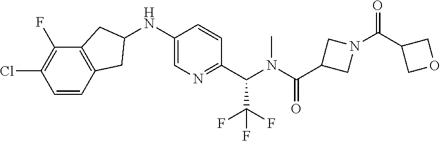 [SMILES: CN(C(=O)C1CN(C(=O)C2COC2)C1)[C@@H](c1ccc(NC2Cc3ccc(Cl)c(F)c3C2)cn1)C(F)(F)F]

### Example 371: N-((1S)-1-(5-((5-Chloro-4-fluoro-2,3-dihydro-1H-inden-2-yl)amino)pyridin-2-yl)-2,2,2-trifluoroethyl)-N-methyl-1-(oxetane-3-carbonyl)azetidine-3-carboxamide—Isomer 2

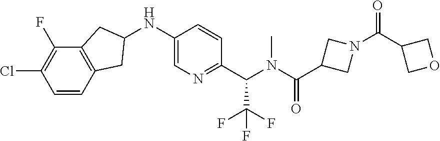 [SMILES: CN(C(=O)C1CN(C(=O)C2COC2)C1)[C@@H](c1ccc(NC2Cc3ccc(Cl)c(F)c3C2)cn1)C(F)(F)F]

To a stirred solution of oxetane-3-carboxylic acid (24 mg, 0.23 mmol, CAS 114012-41-8), DIPEA (0.15 mL, 0.88 mmol) and Intermediate 239 (0.10 g, 0.22 mmol) in DMF (1.1 mL) was added HATU (97 mg, 0.26 mmol) and the mixture was stirred at RT for 3 h. The mixture was concentrated under reduced pressure. The crude product was purified by preparative HPLC (Gemini-NX C18, 21×250 mm×5 μm, flow rate: 30 mL/min, 0.1% aqueous formic acid with MeCN 63% isocratic over 11 min, ramped to 95% over 0.2 min and held for 1.8 min). Chiral purification was carried out by preparative SFC (Chiralpak®-AS-H, 30×250 mm×5 μm, temp 30° C., Back Pressure 100 bar, flow rate: 100 g/min, 78% CO₂ with 22% MeOH modifier) to provide Example 370 (8 mg) as Peak 1, Example 371 (9 mg) as Peak 2. Example 370: ¹H NMR (400 MHz; DMSO-d₆) δ: 8.03-8.00 (m, 1H), 7.37 (dd, 1H), 7.25-7.13 (m, 2H), 7.12-7.01 (m, 1H), 6.59-6.54 (m, 1H), 6.39-5.63 (m, 1H), 4.64-4.56 (m, 4H), 4.36-4.34 (m, 1H), 4.23-4.04 (m, 3H), 3.93-3.83 (m, 3H), 3.44-3.31 (m, 2H), 2.87-2.74 (m, 5H). LCMS (Method H): 541.3 [M+H]⁺. Example 371: ¹H NMR (400 MHz; DMSO-d₆) δ: 8.03-8.00 (m, 1H), 7.37 (dd, 1H), 7.25-7.11 (m, 2H), 7.06-7.01 (m, 1H), 6.59-6.54 (m, 1H), 6.39-5.63 (m, 1H), 4.64-4.56 (m, 4H), 4.37-4.33 (m, 1H), 4.23-4.04 (m, 3H), 3.93-3.83 (m, 3H), 3.44-3.31 (m, 2H), 2.87-2.74 (m, 5H). LCMS (Method H): 541.3 [M+H]⁺.

### Example 372: N-((1S)-1-(5-((5-Chloro-4-methyl-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 1

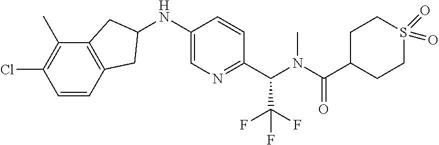 [SMILES: Cc1c(Cl)ccc2c1CC(Nc1ccc([C@H](N(C)C(=O)C3CCS(=O)(=O)CC3)C(F)(F)F)nc1)C2]

### Example 373: N-((1S)-1-(5-((5-Chloro-4-methyl-2,3-dihydro-1H-inden-2-yl)amino)pyridin-2-yl)-2,2,2-trifluoroethyl)-N-methyltetrahydro-2H-thiopyran-4-carboxamide 1,1-dioxide—Isomer 2

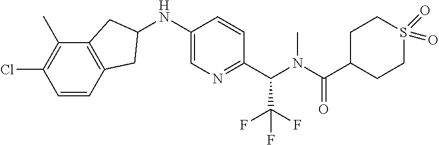 [SMILES: Cc1c(Cl)ccc2c1CC(Nc1ccc([C@H](N(C)C(=O)C3CCS(=O)(=O)CC3)C(F)(F)F)nc1)C2]

The title compound (4.4 mg) was prepared in an analogous manner to Intermediate 193 from Intermediate 229 (0.10 g, 0.17 mmol), methylboronic acid (20 mg, 0.34 mmol), Pd(dppf)Cl₂ (12.3 mg, 0.02 mmol) and K₃PO₄ (0.11 g, 0.50 mmol) in toluene (1.0 mL) and water (0.2 mL) at 90° C. for 16 h. Purified by preparative HPLC (Gemini-NX C18, 21×250 mm×5 μm, flow rate: 30 mL/min, 0.1% aqueous formic acid with MeCN 60% isocratic over 11 min, ramped to 95% over 0.2 min and held for 1.8 min). Chiral purification was carried out by preparative SFC (Chiralpak®-AD-H, 30×250 mm×5 μm, temp 30° C., Back Pressure 100 bar, flow rate: 100 g/min, 60% CO₂ with 40% (1:1 MeCN/MeOH) modifier) to provide Example 372 (18 mg) as Peak 1, Example 373 (21 mg) as Peak 2. Example 372: ¹H NMR (400 MHz; DMSO-d₆) δ: 8.05-8.01 (m, 1H), 7.27-7.20 (m, 1H), 7.14 (d, 1H), 7.08 (d 1H), 7.02 (dd, 1H), 6.54-6.48 (m, 1H), 6.41-6.00 (m, 1H), 4.30-4.25 (m, 1H), 3.38-3.27 (m, 1H), 3.29-3.08 (m, 6H), 3.02 (s, 3H), 2.84-2.60 (m, 2H), 2.25 (s, 3H), 2.19-1.95 (m, 4H). LCMS (Method H): 530.3 [M+H]⁺. Example 373: ¹H NMR (400 MHz; DMSO-d₆) δ: 8.05-8.01 (m, 1H), 7.27-7.20 (m, 1H), 7.14 (d, 1H), 7.08 (d, 1H), 7.02 (dd, 1H), 6.54-6.48 (m, 1H), 6.40-6.01 (m, 1H), 4.30-4.25 (m, 1H), 3.38-3.32 (m, 1H), 3.24-3.08 (m, 6H), 3.02 (s, 3H), 2.84-2.68 (m, 2H), 2.25 (s, 3H), 2.10-1.90 (m, 4H). LCMS (Method H): 530.3 [M+H]⁺.

### Example 374: (1,3-trans)-N-((1S)-1-(5-((5-Chloro-4-fluoro-2,3-dihydro-1H-inden-2-yl)amino)pyridin-2-yl)-2,2,2-trifluoroethyl)-3-hydroxy-N-methylcyclobutane-1-carboxamide—Isomer 1

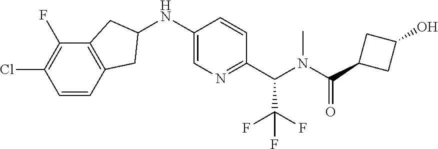 [SMILES: CN(C(=O)[C@H]1C[C@H](O)C1)[C@@H](c1ccc(NC2Cc3ccc(Cl)c(F)c3C2)cn1)C(F)(F)F]

### Example 375: (1,3-trans)-N-((1S)-1-(5-((5-Chloro-4-fluoro-2,3-dihydro-1H-inden-2-yl)amino)pyridin-2-yl)-2,2,2-trifluoroethyl)-3-hydroxy-N-methylcyclobutane-1-carboxamide—Isomer 2

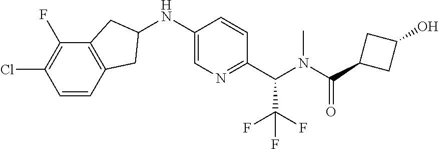 [SMILES: CN(C(=O)[C@H]1C[C@H](O)C1)[C@@H](c1ccc(NC2Cc3ccc(Cl)c(F)c3C2)cn1)C(F)(F)F]

The title compound was prepared in an analogous manner to Example 16 from Intermediate 240 (0.20 g, 0.34 mmol), TBAF (1M in THF, 0.68 mL, 0.68 mmol) in THF (0.5 mL) at RT for 1 h. Chiral purification was carried out by preparative SFC (Chiralpak®-AD-H, 30×250 mm×5 μm, temp 30° C., Back Pressure 100 bar, flow rate: 100 g/min, 73% CO₂ with 27% (1:1 MeCN/MeOH) modifier) to provide Example 374 (19 mg) as Peak 1, Example 375 (26 mg) as Peak 2. Example 374: ¹H NMR (400 MHz; DMSO-d₆) δ: 8.03-8.00 (m, 1H), 7.37 (dd, 1H), 7.22-7.12 (m, 2H), 7.06-7.01 (m, 1H), 6.56-6.51 (m, 1H), 6.42-5.58 (m, 1H), 5.12-5.08 (m, 1H), 4.35-4.33 (m, 1H), 4.15 (dd, 1H), 3.44-3.34 (m, 2H), 3.28-3.26 (m, 1H), 2.88-2.81 (m, 5H), 2.38-2.33 (m, 2H), 2.13-2.04 (m, 2H). LCMS (Method H): 472.3 [M+H]⁺. Example 375: ¹H NMR (400 MHz; DMSO-d₆) δ: 8.02-8.00 (m, 1H), 7.37 (dd, 1H), 7.22-7.11 (m, 2H), 7.06-7.00 (m, 1H), 6.56-6.52 (m, 1H), 6.43-5.59 (m, 1H), 5.12-5.07 (m, 1H), 4.35-4.34 (m, 1H), 4.15 (dd, 1H), 3.44-3.35 (m, 2H), 3.29-3.28 (m, 1H), 2.88-2.81 (m, 5H), 2.38-2.32 (m, 2H), 2.13-2.04 (m, 2H). LCMS (Method H): 472.4 [M+H]⁺.

### Example 376: 1-Acetyl-N-((1S)-1-(5-((4,5-dichloro-2,3-dihydro-1H-inden-2-yl)amino)pyridin-2-yl)-2,2,2-trifluoroethyl)-N-methylazetidine-3-carboxamide—Isomer 1

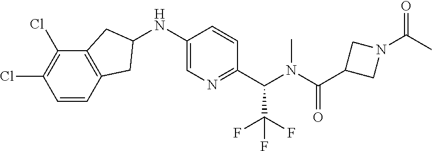 [SMILES: CC(=O)N1CC(C(=O)N(C)[C@@H](c2ccc(NC3Cc4ccc(Cl)c(Cl)c4C3)cn2)C(F)(F)F)C1]

### Example 377: 1-Acetyl-N-((1S)-1-(5-((4,5-dichloro-2,3-dihydro-1H-inden-2-yl)amino)pyridin-2-yl)-2,2,2-trifluoroethyl)-N-methylazetidine-3-carboxamide—Isomer 2

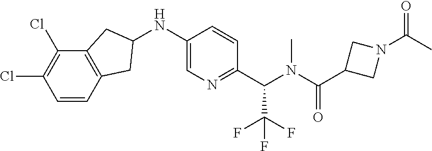 [SMILES: CC(=O)N1CC(C(=O)N(C)[C@@H](c2ccc(NC3Cc4ccc(Cl)c(Cl)c4C3)cn2)C(F)(F)F)C1]

The title compounds were prepared in an analogous manner to Intermediate 3 from Intermediate 228 (0.20 g, 0.47 mmol), triethylamine (0.2 mL, 1.40 mmol), T3P® (50% EtOAc, 0.09 mL, 4.66 mmol) and 1-acetylazetidine-3-carboxylic acid (0.26 g, 1.81 mmol, CAS 97628-91-6) in DCM (2 mL) at RT for 16 h. Purified by flash chromatography (silica gel, eluting 0-20% MeOH in DCM). Chiral purification was carried out by preparative SFC (Chiralpak®-IH, 30×250 mm×5 μm, temp 30° C., Back Pressure 100 bar, flow rate: 100 g/min, 75% CO₂ with 25% (10 mM NH₄HCO₃ in MeOH) modifier) to provide Example 376 (21 mg) as Peak 1, Example 377 (23 mg) as Peak 2. Example 376: ¹H NMR (400 MHz; DMSO-d₆) δ: 8.04-8.01 (m, 1H), 7.44 (d, 1H), 7.25 (d, 1H), 7.18 (dd, 1H), 7.07-7.01 (m, 1H), 6.59-6.54 (m, 1H), 6.41-5.67 (m, 1H), 4.33-4.17 (m, 3H), 4.08-4.00 (m, 1H), 3.88-3.80 (m, 2H), 3.46-3.37 (m, 2H), 2.92-2.75 (m, 5H), 1.76-1.75 (m, 3H). LCMS (Method A): 515.4 [M+H]⁺. Example 377: ¹H NMR (400 MHz; DMSO-d₆) δ: 8.04-8.01 (m, 1H), 7.44 (d, 1H), 7.25 (d, 1H), 7.18 (dd, 1H), 7.07-7.01 (m, 1H), 6.59-6.54 (m, 1H), 6.41-5.67 (m, 1H), 4.35-4.17 (m, 3H), 4.06-4.00 (m, 1H), 3.88-3.80 (m, 2H), 3.46-3.40 (m, 2H), 2.91-2.75 (m, 5H), 1.77-1.74 (m, 3H). LCMS (Method A): 515.4 [M+H]⁺.

### Example 378: N-((1S)-1-(5-((4,5-Dichloro-2,3-dihydro-1H-inden-2-yl)amino)pyridin-2-yl)-2,2,2-trifluoroethyl)-1-(2-hydroxyacetyl)-N-methylazetidine-3-carboxamide—Isomer 1

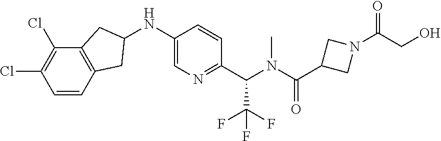 [SMILES: CN(C(=O)C1CN(C(=O)CO)C1)[C@@H](c1ccc(NC2Cc3ccc(Cl)c(Cl)c3C2)cn1)C(F)(F)F]

### Example 379: N-((1S)-1-(5-((4,5-Dichloro-2,3-dihydro-1H-inden-2-yl)amino)pyridin-2-yl)-2,2,2-trifluoroethyl)-1-(2-hydroxyacetyl)-N-methylazetidine-3-carboxamide—Isomer 2

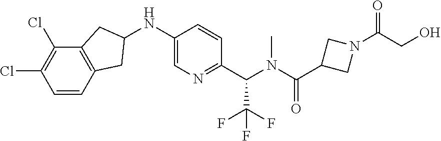 [SMILES: CN(C(=O)C1CN(C(=O)CO)C1)[C@@H](c1ccc(NC2Cc3ccc(Cl)c(Cl)c3C2)cn1)C(F)(F)F]

To a stirred solution of Intermediate 243 (0.13 g, 0.19 mmol) in DCM (1.3 mL) was added TFA (0.04 mL) at RT for 3 h. The mixture was concentrated under reduced pressure then diluted with aqueous NaHCO₃ and extracted into EtOAc. The combined organics were dried over Na₂SO₄, filtered and concentrated under reduced pressure. The crude was purified by flash chromatography (silica gel, eluting 0-80% EtOAc in hexane). Chiral purification was carried out by preparative SFC (Chiralpak®-AS-H, 30×250 mm×5 μm, temp 30° C., Back Pressure 100 bar, flow rate: 100 g/min, 70% CO₂ with 30% MeOH modifier) to provide Example 378 (15 mg) as Peak 1, Example 379 (18 mg) as Peak 2. Example 378: ¹H NMR (400 MHz; DMSO-d₆) δ: 8.03-8.01 (m, 1H), 7.44 (d, 1H), 7.25 (d, 1H), 7.19 (dd, 1H), 7.07-7.01 (m, 1H), 6.59-6.54 (m, 1H), 6.41-5.65 (m, 1H), 4.97 (dd, 1H), 4.43-4.32 (m, 3H), 4.28-4.03 (m, 1H), 3.95-3.86 (m, 4H), 3.46-3.36 (m, 2H), 2.92-2.76 (m, 5H). LCMS (Method H): 531.3 [M+H]⁺. Example 379: ¹H NMR (400 MHz; DMSO-d₆) δ: 8.04-8.01 (m, 1H), 7.43 (d, 1H), 7.25 (d, 1H), 7.18 (dd, 1H), 7.07-7.01 (m, 1H), 6.59-6.54 (m, 1H), 6.41-5.66 (m, 1H), 4.97 (dd, 1H), 4.43-4.24 (m, 3H), 4.11-4.05 (m, 1H), 3.96-3.86 (m, 4H), 3.46-3.36 (m, 2H), 2.92-2.76 (m, 5H). LCMS (Method H): 531.3 [M+H]⁺.

### Example 380: N-((1S)-1-(5-((4,5-Dichloro-2,3-dihydro-1H-inden-2-yl)amino)pyridin-2-yl)-2,2,2-trifluoroethyl)-N-methyl-2-(methylsulfonamido)acetamide—Isomer 1

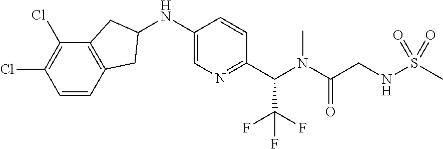 [SMILES: CN(C(=O)CNS(C)(=O)=O)[C@@H](c1ccc(NC2Cc3ccc(Cl)c(Cl)c3C2)cn1)C(F)(F)F]

### Example 381: N-((1S)-1-(5-((4,5-Dichloro-2,3-dihydro-1H-inden-2-yl)amino)pyridin-2-yl)-2,2,2-trifluoroethyl)-N-methyl-2-(methylsulfonamido)acetamide—Isomer 2

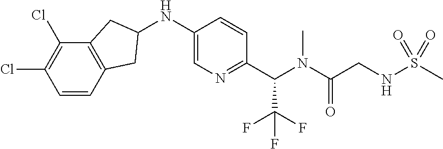 [SMILES: CN(C(=O)CNS(C)(=O)=O)[C@@H](c1ccc(NC2Cc3ccc(Cl)c(Cl)c3C2)cn1)C(F)(F)F]

The title compounds were prepared in an analogous manner to Intermediate 3 from Intermediate 228 (0.15 g, 0.35 mmol), pyridine (0.5 mL), T3P® (50% EtOAc, 1.50 mL, 2.55 mmol) and 2-(methanesulfonamido)acetic acid (0.12 g, 0.76 mmol, CAS 35688-18-7) at RT for 16 h. Purified by flash chromatography (silica gel, eluting 0-45% EtOAc in hexane). Chiral purification was carried out by preparative SFC (Chiralpak®-IH, 30×250 mm×5 μm, temp 30° C., Back Pressure 100 bar, flow rate: 100 g/min, 73% CO₂ with 27% (10 mM NH₄HCO₃ in MeOH) modifier) to provide Example 380 (10 mg) as Peak 1, Example 381 (13 mg) as Peak 2. Example 380: ¹H NMR (400 MHz; DMSO-d₆) δ: 8.09-8.01 (m, 1H), 7.44 (d, 1H), 7.34-7.26 (m, 2H), 7.19 (d, 1H), 7.03 (dd, 1H), 6.59-6.55 (m, 1H), 6.38-5.89 (m, 1H), 4.33-4.35 (m, 1H), 4.22-4.04 (m, 2H), 3.46-3.37 (m, 2H), 2.96-2.74 (m, 8H). LCMS (Method K): 525.3 [M+H]⁺. Example 381: ¹H NMR (400 MHz; DMSO-d₆) δ: 8.09-8.02 (m, 1H), 7.43 (d, 1H), 7.34-7.24 (m, 2H), 7.19 (d, 1H), 7.03 (dd, 1H), 6.59-6.55 (m, 1H), 6.38-5.89 (m, 1H), 4.35-4.33 (m, 1H), 4.22-4.04 (m, 2H), 3.46-3.36 (m, 2H), 2.96-2.82 (m, 8H). LCMS (Method K): 525.3 [M+H]⁺.

### Example 382: (1,3-cis)-N-((1S)-1-(5-((5-Chloro-4-fluoro-2,3-dihydro-1H-inden-2-yl)amino)pyridin-2-yl)-2,2,2-trifluoroethyl)-3-hydroxy-N-methylcyclobutane-1-carboxamide—Isomer 1

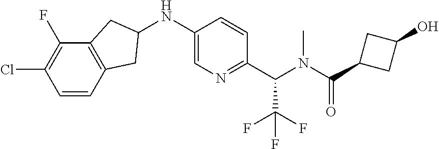 [SMILES: CN(C(=O)[C@H]1C[C@@H](O)C1)[C@@H](c1ccc(NC2Cc3ccc(Cl)c(F)c3C2)cn1)C(F)(F)F]

### Example 383: (1,3-cis)-N-((1S)-1-(5-((5-Chloro-4-fluoro-2,3-dihydro-1H-inden-2-yl)amino)pyridin-2-yl)-2,2,2-trifluoroethyl)-3-hydroxy-N-methylcyclobutane-1-carboxamide—Isomer 2

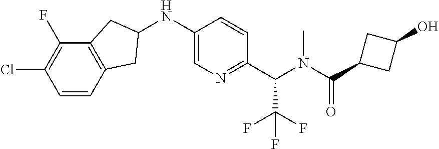 [SMILES: CN(C(=O)[C@H]1C[C@@H](O)C1)[C@@H](c1ccc(NC2Cc3ccc(Cl)c(F)c3C2)cn1)C(F)(F)F]

The title compound was prepared in an analogous manner to Example 16 from Intermediate 244 (0.16 g, 0.25 mmol), TBAF (1M in THF, 0.51 mL, 0.51 mmol) in THF (8 mL) at RT for 1 h. Purified by preparative SFC (DCPAK P4VP, 30×250 mm×5 μm, temp 30° C., Back Pressure 100 bar, flow rate: 100 g/min, 73% CO₂ with 27% MeOH modifier). Chiral purification was carried out by preparative SFC (Chiralpak®-AD-H, 30×250 mm×5 μm, temp 30° C., Back Pressure 100 bar, flow rate: 100 g/min, 65% CO₂ with 35% MeOH modifier) to provide Example 382 (21 mg) as Peak 1, Example 383 (21 mg) as Peak 2. Example 382: ¹H NMR (400 MHz; DMSO-d₆) δ: 8.01 (d, 1H), 7.37 (dd, 1H), 7.21-7.11 (m, 2H), 7.06-7.00 (m, 1H), 6.55-6.51 (m, 1H), 6.40-5.68 (m, 1H), 5.10-5.08 (m, 1H), 4.34 (dd, 1H), 4.01-3.93 (m, 1H), 3.44-3.35 (m, 2H), 3.03-2.71 (m, 6H), 2.43-2.36 (m, 2H), 2.02-1.91 (m, 2H). LCMS (Method H): 472.3 [M+H]⁺. Example 383: ¹H NMR (400 MHz; DMSO-d₆) δ: 8.00 (d, 1H), 7.37 (dd, 1H), 7.21-7.11 (m, 2H), 7.06-7.00 (m, 1H), 6.55-6.52 (m, 1H), 6.40-5.69 (m, 1H), 5.11-5.08 (m, 1H), 4.34 (dd, 1H), 4.00-3.93 (m, 1H), 3.44-3.31 (m, 2H), 3.08-2.71 (m, 6H), 2.43-2.37 (m, 2H), 2.00-1.90 (m, 2H). LCMS (Method H): 472.3 [M+H]⁺.

### Example 384: N-((1S)-1-(5-((5-Chloro-4-fluoro-2,3-dihydro-1H-inden-2-yl)amino)pyridin-2-yl)-2,2,2-trifluoroethyl)-N-methyl-1-(oxetane-2-carbonyl)azetidine-3-carboxamide—Isomer 1

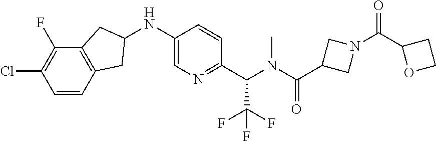 [SMILES: CN(C(=O)C1CN(C(=O)C2CCO2)C1)[C@@H](c1ccc(NC2Cc3ccc(Cl)c(F)c3C2)cn1)C(F)(F)F]

### Example 385: N-((1S)-1-(5-((5-Chloro-4-fluoro-2,3-dihydro-1H-inden-2-yl)amino)pyridin-2-yl)-2,2,2-trifluoroethyl)-N-methyl-1-(oxetane-2-carbonyl)azetidine-3-carboxamide—Isomer 2

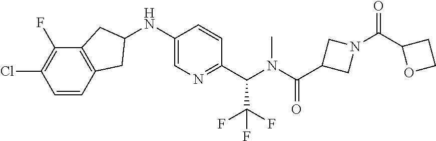 [SMILES: CN(C(=O)C1CN(C(=O)C2CCO2)C1)[C@@H](c1ccc(NC2Cc3ccc(Cl)c(F)c3C2)cn1)C(F)(F)F]

### Example 386: N-((1S)-1-(5-((5-Chloro-4-fluoro-2,3-dihydro-1H-inden-2-yl)amino)pyridin-2-yl)-2,2,2-trifluoroethyl)-N-methyl-1-(oxetane-2-carbonyl)azetidine-3-carboxamide—Isomer 3

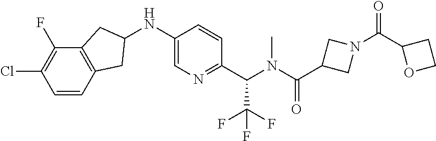 [SMILES: CN(C(=O)C1CN(C(=O)C2CCO2)C1)[C@@H](c1ccc(NC2Cc3ccc(Cl)c(F)c3C2)cn1)C(F)(F)F]

### Example 387: N-((1S)-1-(5-((5-Chloro-4-fluoro-2,3-dihydro-1H-inden-2-yl)amino)pyridin-2-yl)-2,2,2-trifluoroethyl)-N-methyl-1-(oxetane-2-carbonyl)azetidine-3-carboxamide—Isomer 4

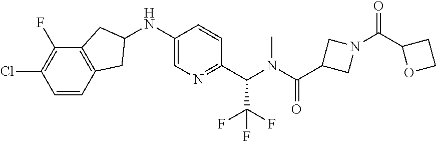 [SMILES: CN(C(=O)C1CN(C(=O)C2CCO2)C1)[C@@H](c1ccc(NC2Cc3ccc(Cl)c(F)c3C2)cn1)C(F)(F)F]

The title compounds were prepared in an analogous manner to Intermediate 3 from Intermediate 239 (0.20 g, 0.34 mmol), triethylamine (3 mL, 21.5 mmol), T3P® (50% EtOAc, 2 mL) and oxetane-2-carboxylic acid (74 mg, 0.69 mmol, CAS 864373-47-7) in DCM (1.5 mL) at RT for 16 h. Purified by flash chromatography (silica gel, eluting 0-15% MeOH in DCM). Chiral purification was carried out by preparative SFC (Chiralpak®-IC, 30×250 mm×5 μm, temp 30° C., Back Pressure 100 bar, flow rate: 100 g/min, 55% CO₂ with 45% MeOH modifier) to provide Example 384 (20 mg) as Peak 1, Example 385 (12 mg) as Peak 2, Example 386 (14 mg) as Peak 3, Example 387 (8 mg) as Peak 4. Example 384: ¹H NMR (400 MHz; DMSO-d₆) δ: 8.01 (br s, 1H), 7.37 (dd, 1H), 7.24-7.17 (m, 1H), 7.13 (d, 1H), 7.08-7.00 (m, 1H), 6.59-6.54 (m, 1H), 6.39-5.67 (m, 1H), 5.14 (dd, 1H), 4.56-3.91 (m, 8H), 3.39 (dd, 2H), 2.87-2.76 (m, 6H), 2.67-2.62 (m, 1H). LCMS (Method A): 541.4 [M+H]⁺. Example 385: ¹H NMR (400 MHz; DMSO-d₆) δ: 8.06-8.00 (m, 1H), 7.37 (dd, 1H), 7.24-7.16 (m, 1H), 7.12 (d, 1H), 7.07-7.00 (m, 1H), 6.58-6.54 (m, 1H), 6.39-5.67 (m, 1H), 5.14 (dd, 1H), 4.56-3.91 (m, 8H), 3.39 (dd, 2H), 2.87-2.75 (m, 6H), 2.70-2.60 (m, 1H). LCMS (Method A): 541.4 [M+H]⁺. Example 386: ¹H NMR (400 MHz; DMSO-d₆) δ: 8.03-8.00 (m, 1H), 7.37 (dd, 1H), 7.24-7.16 (m, 1H), 7.12 (d, 1H), 7.08-7.00 (m, 1H), 6.59-6.54 (m, 1H), 6.39-5.65 (m, 1H), 5.14 (dd, 1H), 4.57-3.88 (m, 8H), 3.39 (dd, 2H), 2.87-2.76 (m, 6H), 2.69-2.60 (m, 1H). LCMS (Method A): 541.4 [M+H]⁺. Example 387: ¹H NMR (400 MHz; DMSO-d₆) δ: 8.02-8.00 (m, 1H), 7.37 (dd, 1H), 7.24-7.16 (m, 1H), 7.12 (d, 1H), 7.08-7.01 (m, 1H), 6.59-6.54 (m, 1H), 6.41-5.65 (m, 1H), 5.14 (dd, 1H), 4.57-3.89 (m, 8H), 3.39 (dd, 2H), 2.87-2.75 (m, 6H), 2.68-2.60 (m, 1H). LCMS (Method A): 541.4 [M+H]⁺.

### Example 388: (3S)—N-((1S)-1-(6-((5-Chloro-4-fluoro-2,3-dihydro-1H-inden-2-yl)amino)pyridin-3-yl)-2,2,2-trifluoroethyl)-N-methyl-6-oxopiperidine-3-carboxamide—Isomer 1

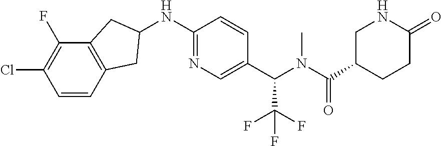 [SMILES: CN(C(=O)[C@H]1CCC(=O)NC1)[C@@H](c1ccc(NC2Cc3ccc(Cl)c(F)c3C2)nc1)C(F)(F)F]

### Example 389: (3S)—N-((1S)-1-(6-((5-Chloro-4-fluoro-2,3-dihydro-1H-inden-2-yl)amino)pyridin-3-yl)-2,2,2-trifluoroethyl)-N-methyl-6-oxopiperidine-3-carboxamide—Isomer 2

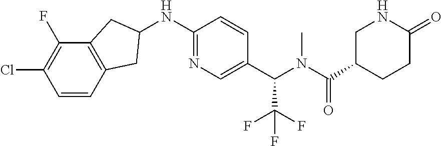 [SMILES: CN(C(=O)[C@H]1CCC(=O)NC1)[C@@H](c1ccc(NC2Cc3ccc(Cl)c(F)c3C2)nc1)C(F)(F)F]

To a stirred solution of Intermediate 245 (0.14 g, 0.18 mmol) in DCM (3 mL) was added TFA (0.14 mL) at RT for 2 h. The mixture was concentrated under reduced pressure. The crude was purified by preparative SFC (DCPAK P4VP, 30×250 mm×5 μm, temp 30° C., Back Pressure 100 bar, flow rate: 100 g/min, 75% CO₂ with 25% MeOH modifier). Chiral purification was carried out by preparative SFC (ChiraICEL-OX-H, 30×250 mm×5 μm, temp 30° C., Back Pressure 100 bar, flow rate: 100 g/min, 65% CO₂ with 35% MeOH modifier) to provide Example 388 (12 mg) as Peak 1, Example 389 (11 mg) as Peak 2. Example 388: ¹H NMR (400 MHz; DMSO-d₆) δ: 8.09-7.98 (m, 1H), 7.49 (br s, 1H), 7.39 (d, 1H), 7.35 (d, 1H), 7.25 (d, 1H), 7.12 (d, 1H), 6.56-6.53 (m, 1H), 6.40-6.16 (m, 1H), 4.67 (dd, 1H), 3.38-3.15 (m, 5H), 2.95 (s, 3H), 2.88 (dd, 2H), 2.26-2.18 (m, 2H), 1.85-1.74 (m, 2H). LCMS (Method H): 499.3 [M+H]⁺. Example 389: ¹H NMR (400 MHz; DMSO-d₆) δ: 8.09-7.99 (m, 1H), 7.49 (br s, 1H), 7.43 (d, 1H), 7.37 (d, 1H), 7.24 (d, 1H), 7.11 (d, 1H), 6.57-6.530 (m, 1H), 6.40-6.15 (m, 1H), 4.67 (dd, 1H), 3.39-3.14 (m, 5H), 2.95 (s, 3H), 2.91-2.83 (m, 2H), 2.24-2.18 (m, 2H), 1.84-1.75 (m, 2H). LCMS (Method H): 499.3 [M+H]⁺.

### Biological Assays

### MALT1 Protease Assay 1

MALT1 protease activity was assessed in vitro by measuring the cleavage of a fluorogenic tetrapeptide substrate.

A protein (MALT1-GS-Ub) comprising 6-His-hMALT-1 (residues 339-715), fused to ubiquitin with an 8×GGS linker, was expressed in E. coli, and purified using chitin resin to remove contaminants followed by affinity chromatography purification and size-exclusion chromatography, according to standard protocols.

Briefly, MALT1-GS-Ub (300-600 nM) was incubated with substrate (Ac-LVSR-AMC, 100 μM) in reaction buffer comprising 50 mM HEPES (pH7.0), 25 mM KCl, 0.1% (v/v) CHAPS and 1 mM TCEP.

Test compounds dissolved in DMSO were dispensed into assay plates (384-well, black, shallow ProxiPlates). 7 μl enzyme solution was added and incubated at room temperature for 30 minutes to allow compound binding to occur. 2 μl substrate solution was then added and the fluorescence (excitation 360 nm, emission 460 nm) read every 15 minutes using a suitable plate reader. Final assay DMSO concentration was 1%. Linearity over 60 minutes was confirmed, and assay signal was calculated by subtracting raw counts at time 0 from those at 60 minutes. % inhibition was calculated for each well, using no enzyme for 100% inhibition controls and no compound for 0% inhibition controls. IC50 values were calculated using GraphPad Prism using a 4-parameter non-linear regression curve fit.

### MALT1 Protease Assay 2

MALT1 protease activity was assessed in vitro by measuring the cleavage of a fluorogenic tetrapeptide substrate.

For a higher sensitivity assay, commercially available FL hMALT1 enzyme (Abcam, 1-10 nM) was incubated with substrate (Ac-LVSR-AMC, 100 μM) in reaction buffer comprising 50 mM HEPES (pH7.0), 25 mM KCl, 0.1% (v/v) CHAPS, 1 mM TCEP and 0.7 M sodium citrate.

Test compounds dissolved in DMSO were dispensed into assay plates (384-well, black, shallow ProxiPlates). 7 μl enzyme solution was added and incubated at room temperature for 30 minutes to allow compound binding to occur. 2 μl substrate solution was then added and the fluorescence (excitation 360 nm, emission 460 nm) read every 15 minutes using a suitable plate reader. Final assay DMSO concentration was 1%. Linearity over 60 minutes was confirmed, and assay signal was calculated by subtracting raw counts at time 0 from those at 60 minutes. % inhibition was calculated for each well, using no enzyme for 100% inhibition controls and no compound for 0% inhibition controls. IC₅₀ values were calculated using GraphPad Prism using a 4-parameter non-linear regression curve fit.

### Human IL-2 Release Phenotypic Assay (Assay 3)

This assay measured the inhibition by MALT1 inhibitors of the IL-2 release from stimulated Jurkat cells, a human immortalized T lymphocyte cell line. IL-2 production from Jurkat cells is regulated by activation of NFκB signalling, which is in turn regulated by MALT1 protease activity.

Jurkat cells (clone E6-1, ATCC) were cultured in RPMI1640 supplemented with 10% (v/v) FBS and 1% (v/v) penicillin/streptomycin (100× liquid stocks). Cells were seeded in 96-well white tissue culture-treated plates (Perkin Elmer) at 50 000 cells/well and incubated overnight at 37° C., 5% CO₂. Test compounds in DMSO at half-log dilutions were added to the cells and incubated for 30 mins. Final DMSO concentration was 0.5%. Cells were then stimulated with 200 ng/mL PMA+300 ng/mL ionomycin and incubated for 48 hours. IL-2 present in the supernatant was then measured by ELISA according to the kit instructions (ELISA MAX Deluxe Set Human IL-2, BioLegend). % inhibition was calculated for each well, using 30 μM of the commercially available MALT1 inhibitor, MLT-748, for 100% inhibition controls and no compound for 0% inhibition controls. IC₅₀ values were calculated using GraphPad Prism using 4-parameter non-linear regression curve fit.

The results of testing the Example compounds in the three in vitro assays described above are shown in Table 1.

TABLE 1
MALT1 assay data

| Example | Assay 1 | Assay 2 | Assay 3 |
|---|---|---|---|
| Number | IC₅₀ (μM) | IC₅₀ (μM) | IC₅₀ (μM) |
| 1 | 0.14 | 0.007 | 0.43 |
| 2 | 0.14 | 0.05 | 0.62 |
| 3 | 0.34 | 0.12 | 1.94 |
| 4 | 0.14 | 0.034 | 0.069 |
| 5 | 0.14 | 0.25 | 2.25 |
| 6 | 0.15 | 0.016 | 0.32 |
| 7 | 0.19 | 0.068 | 0.82 |
| 8 | 0.20 | 0.039 | 0.15 |
| 9 | 0.24 | 0.006 | 0.21 |
| 10 | 0.54 | 0.12 | 1.55 |
| 11 | 3.89 | 1.20 | >10 |
| 12 | 0.18 | 0.29 | 0.49 |
| 13 | 0.40 | 0.40 | 1.93 |
| 14 | 0.22 | 0.11 | 0.32 |
| 15 | 0.29 | 0.42 | 3.1 |
| 16 | 0.24 | 0.17 | 0.25 |
| 17 | 0.48 | 4.5 | 2.95 |
| 18 | 0.33 | 0.12 | 0.52 |
| 19 | 0.17 | 0.030 | 0.11 |
| 20 | 0.21 | 0.24 | 0.51 |
| 21 | 0.20 | 0.062 | 0.26 |
| 22 | 0.29 | 0.45 | 1.73 |
| 23 | 0.39 | 0.33 | 1.08 |
| 24 | 0.16 | 0.089 | 0.87 |
| 25 | 0.16 | 0.027 | 0.061 |
| 26 | 0.36 | 0.70 | 0.91 |
| 27 | 0.19 | 0.18 | 0.40 |
| 28 | 0.29 | 0.20 | 2.61 |
| 29 | 0.16 | 0.014 | 0.69 |
| 30 | 0.25 | 0.27 | 7.4 |
| 31 | 0.13 | 0.05 | 1.19 |
| 32 | 0.20 | 0.13 | 5.68 |
| 33 | 0.36 | 0.26 | 2.48 |
| 34 | 0.17 | 0.085 | 4.98 |
| 35 | 0.29 | 0.027 | 3.76 |
| 36 | 0.38 | 0.039 | 1.16 |
| 37 | 0.27 | 0.039 | 0.50 |
| 38 | 0.21 | 0.016 | 0.56 |
| 39 | 0.24 | 0.17 | 3.59 |
| 40 | 0.23 | 0.077 | 1.35 |
| 41 | 0.22 | 0.019 | 1.04 |
| 42 | 0.16 | 0.035 | 1.60 |
| 43 | 0.15 | 0.062 | 4.04 |
| 44 | 0.18 | 0.021 | 0.77 |
| 45 | 0.24 | 0.18 | 0.73 |
| 46 | 0.25 | 0.066 | 0.44 |
| 47 | 0.17 | 0.11 | 2.35 |
| 48 | 0.14 | 0.017 | 1.16 |
| 49 | 0.26 | — | 4.69 |
| 50 | 0.12 | — | 2.94 |
| 51 | 0.93 | — | — |
| 52 | 0.26 | — | 9.51 |
| 53 | 0.17 | — | 7.29 |
| 54 | 0.33 | — | 2.11 |
| 55 | 0.52 | 0.22 | 4.57 |
| 56 | 0.15 | 0.014 | 0.093 |
| 57 | 0.16 | 0.091 | 3.69 |
| 58 | 0.19 | 0.038 | 1.02 |
| 59 | 2.71 | — | — |
| 60 | 0.21 | 0.053 | 1.1 |
| 61 | 0.18 | — | 0.84 |
| 62 | 0.17 | — | 2.12 |
| 63 | 0.39 | 0.20 | 2.55 |
| 64 | 0.15 | 0.012 | 0.29 |
| 65 | 0.19 | 0.045 | 2.24 |
| 66 | 0.22 | 0.020 | 0.59 |
| 67 | 0.45 | 0.078 | 5.97 |
| 68 | 0.19 | 0.015 | 0.12 |
| 69 | 0.28 | 0.079 | 0.92 |
| 70 | 0.43 | 0.81 | 3.5 |
| 71 | 0.33 | — | 2.62 |
| 72 | 0.27 | 0.22 | 0.52 |
| 73 | 0.42 | 0.007 | 0.37 |
| 74 | 0.32 | 0.018 | 0.18 |
| 75 | 0.25 | 0.37 | 0.82 |
| 76 | 0.22 | 0.036 | 0.50 |
| 77 | 0.31 | 0.068 | 0.92 |
| 78 | 0.28 | 0.018 | 0.156 |
| 79 | 0.61 | 0.73 | 4.31 |
| 80 | 0.27 | 0.21 | 2.24 |
| 81 | 1.44 | 0.80 | 3.86 |
| 82 | 0.47 | 0.46 | 2.99 |
| 83 | 16 | >3 | — |
| 84 | 0.32 | 0.18 | 0.64 |
| 85 | 0.61 | 0.62 | 1.94 |
| 86 | 11 | >3 | — |
| 87 | 1.22 | 0.47 | 1.13 |
| 88 | 0.65 | 0.37 | 1.71 |
| 89 | 6.86 | >3 | — |
| 90 | 81 | >3 | — |
| 91 | 12 | >3 | — |
| 92 | 0.50 | 0.28 | 0.87 |
| 93 | 1.0 | 0.69 | 3.04 |
| 94 | 17 | >3 | — |
| 95 | 0.58 | 0.64 | 1.30 |
| 96 | 0.14 | 0.13 | 1.56 |
| 97 | 16 | >3 | — |
| 98 | 0.21 | 0.59 | 0.88 |
| 99 | 26 | >3 | — |
| 100 | 0.42 | 0.41 | 0.85 |
| 101 | 0.28 | 0.39 | 2.64 |
| 102 | 17 | >3 | — |
| 103 | 7.2 | >3 | — |
| 104 | — | 0.14 | 2.70 |
| 105 | — | 0.036 | 0.23 |
| 106 | — | 0.13 | 0.54 |
| 107 | — | 0.045 | 0.41 |
| 108 | — | 0.15 | 0.90 |
| 109 | — | 0.76 | 0.90 |
| 110 | — | 0.016 | 0.149 |
| 111 | — | 0.052 | 0.65 |
| 112 | — | 0.23 | 0.43 |
| 113 | — | 0.067 | 0.49 |
| 114 | — | 0.19 | 1.02 |
| 115 | — | 0.24 | 0.51 |
| 116 | — | 0.068 | 0.51 |
| 117 | — | 0.24 | 2.06 |
| 118 | — | 0.049 | 0.28 |
| 119 | — | 0.15 | 1.72 |
| 120 | — | 0.77 | — |
| 121 | — | 0.51 | — |
| 122 | — | 1.14 | — |
| 123 | — | 0.87 | — |
| 124 | — | 0.19 | 1.29 |
| 125 | — | 0.062 | 0.33 |
| 126 | — | 0.12 | 0.70 |
| 127 | — | 0.30 | — |
| 128 | — | 0.40 | — |
| 129 | — | 0.21 | 1.80 |
| 130 | — | 0.12 | 1.08 |
| 131 | — | 0.26 | — |
| 132 | — | 0.25 | 1.37 |
| 133 | — | 0.15 | 0.60 |
| 134 | — | 0.16 | 0.71 |
| 135 | — | 0.45 | — |
| 136 | — | 0.036 | 0.31 |
| 137 | — | 0.15 | 1.25 |
| 138 | — | 0.78 | — |
| 139 | — | 0.37 | — |
| 140 | — | 0.18 | 1.27 |
| 141 | — | 0.21 | 1.23 |
| 142 | — | 0.20 | 1.79 |
| 143 | — | 0.68 | — |
| 144 | — | 0.21 | 2.63 |
| 145 | — | 0.23 | — |
| 146 | — | 0.11 | 0.48 |
| 147 | — | 0.26 | — |
| 148 | — | 0.29 | — |
| 149 | — | 0.24 | — |
| 150 | 0.15 | 0.066 | 0.10 |
| 151 | 0.12 | 0.004 | 0.097 |
| 152 | 0.26 | 0.42 | 0.36 |
| 153 | 0.19 | 0.067 | 0.12 |
| 154 | — | 0.15 | |
| 155 | — | 0.18 | |
| 156 | — | 0.17 | 1.72 |
| 157 | — | 0.064 | 0.77 |
| 158 | — | 0.44 | |
| 159 | — | 0.14 | 1.16 |
| 160 | — | 0.031 | 0.18 |
| 161 | — | 0.31 | |
| 162 | — | 0.11 | |
| 163 | — | 0.19 | |
| 164 | — | 0.033 | 0.15 |
| 165 | — | 0.087 | 0.45 |
| 166 | — | 0.083 | 0.51 |
| 167 | — | 0.10 | |
| 168 | — | 0.18 | |
| 169 | — | 0.076 | 0.72 |
| 170 | — | 0.052 | 0.21 |
| 171 | — | 0.21 | |
| 172 | — | 0.071 | 0.49 |
| 173 | — | 0.13 | |
| 174 | — | 0.070 | 0.89 |
| 175 | — | 0.054 | 0.81 |
| 176 | — | 0.19 | |
| 177 | — | 6.6 | |
| 178 | — | 0.26 | |
| 179 | — | 0.088 | 0.47 |
| 180 | — | 0.37 | |
| 181 | — | 0.15 | |
| 182 | — | 0.05 | 0.40 |
| 183 | — | 0.072 | 0.51 |
| 184 | — | 0.15 | |
| 185 | — | 0.042 | 0.30 |
| 186 | — | 0.068 | 0.46 |
| 187 | — | 0.094 | 0.45 |
| 188 | — | 0.047 | 0.65 |
| 189 | — | 0.091 | 3.0 |
| 190 | — | 3.3 | |
| 191 | — | 0.026 | 0.74 |
| 192 | — | 0.06 | 0.44 |
| 193 | — | 0.29 | |
| 194 | — | 0.053 | 0.63 |
| 195 | — | 0.22 | |
| 196 | — | 0.051 | 0.86 |
| 197 | — | 2.6 | |
| 198 | — | 0.078 | 1.2 |
| 199 | — | 0.073 | 0.29 |
| 200 | — | 1.1 | |
| 201 | — | 0.11 | |
| 202 | — | 0.16 | |
| 203 | — | 0.12 | |
| 204 | — | 0.22 | |
| 205 | — | 0.18 | |
| 206 | — | 0.17 | |
| 207 | — | 0.12 | |
| 208 | — | 0.027 | 0.25 |
| 209 | — | 0.052 | 0.38 |
| 210 | — | 0.20 | |
| 211 | — | 0.11 | |
| 212 | — | 0.13 | |
| 213 | — | 0.50 | |
| 214 | — | 0.32 | |
| 215 | — | 0.088 | 0.63 |
| 216 | — | 0.051 | 1.9 |
| 217 | — | 0.17 | |
| 218 | — | 0.089 | 0.43 |
| 219 | — | 0.067 | 0.33 |
| 220 | — | 0.16 | |
| 221 | — | 0.021 | 0.23 |
| 222 | — | 0.026 | 0.26 |
| 223 | — | 0.023 | 0.15 |
| 224 | — | 0.017 | 0.20 |
| 225 | — | 0.063 | 0.52 |
| 226 | — | 0.039 | 0.25 |
| 227 | — | 0.018 | 0.099 |
| 228 | — | 0.057 | 1.0 |
| 229 | — | 0.14 | |
| 230 | — | 0.038 | 0.34 |
| 231 | — | 0.11 | |
| 232 | — | 0.03 | 0.18 |
| 233 | — | 0.020 | 0.17 |
| 234 | — | 0.12 | |
| 235 | — | 0.06 | 1.0 |
| 236 | — | 0.21 | |
| 237 | — | 0.11 | |
| 238 | — | 0.47 | |
| 239 | — | 0.15 | |
| 240 | — | 0.037 | 0.33 |
| 241 | — | 0.025 | 0.40 |
| 242 | — | 0.20 | |
| 243 | — | 0.086 | 0.64 |
| 244 | — | 0.055 | 0.27 |
| 245 | — | 0.13 | |
| 246 | — | 0.33 | |
| 247 | — | 0.03 | 0.17 |
| 248 | — | 0.14 | |
| 249 | — | 0.05 | 0.35 |
| 250 | — | 0.041 | 0.30 |
| 251 | — | 0.032 | 0.32 |
| 252 | — | 0.071 | 0.59 |
| 253 | — | 0.066 | 0.42 |
| 254 | — | 0.053 | 1.4 |
| 255 | — | 0.11 | |
| 256 | — | 1.1 | |
| 257 | — | 2.0 | |
| 258 | — | 4.5 | |
| 259 | — | 0.18 | |
| 260 | — | 0.21 | 0.86 |
| 261 | — | 0.082 | 1.41 |
| 262 | — | 2.68 | 7.7 |
| 263 | — | 0.061 | 0.51 |
| 264 | — | 1.2 | |
| 265 | — | 0.032 | 0.22 |
| 266 | — | 1.6 | |
| 267 | — | 2.2 | |
| 268 | — | 3.4 | |
| 269 | — | 0.15 | |
| 270 | — | 0.081 | 0.27 |
| 271 | — | 3.3 | |
| 272 | — | 2.1 | |
| 273 | — | 0.46 | |
| 274 | — | 0.28 | |
| 275 | — | 0.52 | |
| 276 | — | 0.78 | |
| 277 | — | 0.043 | 0.20 |
| 278 | — | 0.35 | |
| 279 | — | 0.015 | 0.096 |
| 280 | — | 0.86 | |
| 281 | — | 0.014 | 0.46 |
| 282 | — | 0.34 | |
| 283 | — | 0.91 | |
| 284 | — | 0.085 | 0.49 |
| 285 | — | 0.69 | |
| 286 | — | 0.73 | |
| 287 | — | 0.29 | |
| 288 | — | 1.64 | |
| 289 | — | 0.045 | 0.30 |
| 290 | — | 0.12 | |
| 291 | — | 0.34 | |
| 292 | — | 0.84 | |
| 293 | — | 0.055 | 0.46 |
| 294 | — | 2.4 | |
| 295 | — | 1.9 | |
| 296 | — | 1.4 | |
| 297 | — | 0.17 | 0.36 |
| 298 | — | 0.21 | |
| 299 | — | 0.094 | 0.43 |
| 300 | — | 0.25 | 0.35 |
| 301 | — | 0.27 | |
| 302 | — | 0.48 | |
| 303 | — | 0.19 | |
| 304 | — | 0.27 | |
| 305 | — | 0.30 | |
| 306 | — | 0.009 | 0.20 |
| 307 | — | 0.22 | |
| 308 | — | 1.4 | |
| 309 | — | 0.23 | |
| 310 | — | 0.43 | |
| 311 | — | 0.30 | |
| 312 | — | 0.015 | 0.35 |
| 313 | — | 0.19 | |
| 314 | — | 0.15 | 0.38 |
| 315 | — | 0.21 | |
| 316 | — | 1.1 | |
| 317 | — | 0.072 | 0.113 |
| 318 | — | 0.66 | |
| 319 | — | 0.024 | 0.066 |
| 320 | — | 0.75 | |
| 321 | — | 0.025 | 0.086 |
| 322 | — | 0.31 | |
| 323 | — | 0.014 | 0.10 |
| 324 | — | 1.0 | |
| 325 | — | 0.009 | 0.035 |
| 326 | — | 0.24 | |
| 327 | — | 0.57 | |
| 328 | — | 0.018 | 0.24 |
| 329 | — | 0.68 | |
| 330 | — | 0.016 | 0.22 |
| 331 | — | 0.40 | |
| 332 | — | 0.014 | 0.047 |
| 333 | — | 0.16 | |
| 334 | — | 0.30 | |
| 335 | — | 0.014 | 0.042 |
| 336 | — | 0.69 | |
| 337 | — | 0.051 | 0.17 |
| 338 | — | 0.96 | |
| 339 | — | 0.024 | 0.066 |
| 340 | — | 0.56 | |
| 341 | — | 0.015 | 0.10 |
| 342 | — | 1.32 | |
| 343 | — | 0.077 | 0.36 |
| 344 | — | 0.31 | |
| 345 | — | 0.007 | |
| 346 | — | 1.1 | |
| 347 | — | 0.016 | 0.041 |
| 348 | — | 0.41 | |
| 349 | — | 1.04 | |
| 350 | — | 0.061 | 0.23 |
| 351 | — | 0.59 | |
| 352 | — | 0.046 | 0.11 |
| 353 | — | 0.033 | 0.063 |
| 354 | — | 1.0 | |
| 355 | — | 0.15 | |
| 356 | — | 0.21 | |
| 357 | — | 0.69 | |
| 358 | — | 0.055 | 0.25 |
| 359 | — | 0.48 | |
| 360 | — | 0.03 | 0.11 |
| 361 | — | 7.2 | |
| 362 | — | 0.28 | |
| 363 | — | 0.07 | |
| 364 | — | 0.12 | |
| 365 | — | 3.64 | |
| 366 | — | 3.66 | |
| 367 | — | 0.31 | |
| 368 | — | 0.27 | |
| 369 | — | 0.072 | |
| 370 | — | 1.37 | |
| 371 | — | 0.075 | 0.22 |
| 372 | — | 0.019 | 0.13 |
| 373 | — | 0.49 | |
| 374 | — | 0.05 | 0.23 |
| 375 | — | 1.32 | |
| 376 | — | 1.20 | |
| 377 | — | 0.04 | 0.13 |
| 378 | — | 1.29 | |
| 379 | — | 0.046 | |
| 380 | — | 1.87 | |
| 381 | — | 0.077 | |
| 382 | — | 0.095 | |
| 383 | — | 1.60 | |
| 384 | — | 2..09 | |
| 385 | — | 0.13 | |
| 386 | — | 2.67 | |
| 387 | — | 0.31 | |
| 388 | — | 0.49 | |
| 389 | — | 0.040 | |

### IL-2 Release PK/PD Model

Female C57BL/6 mice at approx. 6 weeks of age are allocated into groups based on body weight, and orally dosed with compound at time T −1 hr, in a vehicle comprising 20% HPBCD (w/v), 0.5% Tween 80 (v/v) and 10% (v/v) N,N-di-methyl acetamide in saline, at a volume of 10 mL/kg. One hour later, at T0, they are challenged with an intravenous injection of anti-CD3 antibody (Ultra-LEAF™, #100340 Biolegend), appropriately diluted in sterile PBS to dose 10 μg/mouse in a volume of 100 μL. 4 hours after aCD3 challenge the mice are anaesthetized and terminal blood samples collected from the carotid artery. A 20 μL blood sample is transferred to an EDTA tube, then diluted 1:1 with water and frozen at −20° C. until subsequent bioanalysis by LC-MS/MS. The remaining blood samples are allowed to clot for 30 minutes, centrifuged to separate serum, snap frozen and stored at −80° C. until IL-2 analysis by ELISA (Mouse IL-2 DuoSet ELISA, R&D Systems). IL-2 concentration in the serum is calculated by interpolation from a standard curve. Statistical analysis (ordinary one-way ANOVA and Dunnett's multiple comparison) is performed using GraphPad Prism.

1. A compound according to Formula I, or a pharmaceutically acceptable salt thereof: 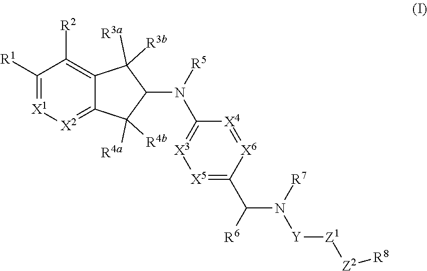
wherein:
X¹ and X² are independently selected from CR⁹ or N;
X³, X⁴, X⁵, and X⁶ are independently selected from CR¹⁰ or N, provided that no more than two of X³, X⁴, X⁵, and X⁶ are N;
Y is 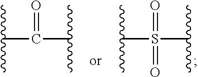
Z¹ is absent, 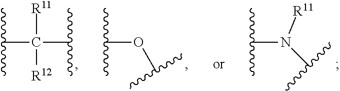
Z² is absent or a C₁₋₄alkylene linker group, optionally substituted with one or more substituents independently selected from C₁₋₄alkyl, C₁₋₄alkoxy, C₁₋₄haloalkyl, halo, cyano, hydroxy, and C₃₋₇cycloalkyl; and optionally spiro attached, to a 3- to 6-membered cycloalkyl or 3- to 6-membered heterocyclyl ring, wherein the cycloalkyl and heterocyclyl ring is optionally substituted with one or more substituents independently selected from C₁₋₄alkyl, C₁₋₄haloalkyl, hydroxy, C₁₋₄alkoxy, C₃₋₆cycloalkyl, halo, and cyano;
R¹ and R² are independently selected from hydrogen, C₁₋₄alkyl, C₁₋₄alkoxy, C₁₋₄haloalkyl, C₁₋₄haloalkoxy, halo, cyano, hydroxy, C₃₋₇cycloalkyl, and S(O)₂C₁₋₄alkyl;
R^{3a}, R^{3b}, R^{4a}, and R^{4b} are independently selected from hydrogen, C₁₋₄alkyl, C₁₋₄haloalkyl, C₃₋₇cycloalkyl, halo, cyano, NR¹³R¹⁴, C(O)OR¹⁵, C(O)NR¹⁶R¹⁷, and OR¹⁸;
R⁵ is hydrogen or C₁₋₃alkyl;
R⁶ is C₁₋₃alkyl, C₁₋₃haloalkyl, or C₃₋₅cycloalkyl;
R⁷ is hydrogen, C₁₋₃alkyl, or CD₃;
R⁸ is selected from OR¹⁹, NR²⁰R²¹, C(O)R²², S(O)₂NR²³R²⁴, cyano, C₃₋₈cycloalkyl, 4- to 10-membered heterocyclyl, 5- to 10-membered heteroaryl, and phenyl, wherein said C₃₋₈cycloalkyl, 4- to 10-membered heterocyclyl, 5- to 10-membered heteroaryl, and phenyl groups are optionally substituted with one or more substituents of formula C₀₋₃alkylene-R²⁵, wherein each R²⁵ is independently selected from C₁₋₄alkyl, hydroxy, C₁₋₄alkoxy, halo, C₁₋₄haloalkyl, C₃₋₆cycloalkyl, 4- to 6-membered heterocyclyl, NR²⁶R²⁷, NR²⁸C(O)R²⁹, C(O)R³⁰, S(O)₂R³¹ and NR³²S(O)₂R³³; and
wherein said C₃₋₈cycloalkyl and 4- to 10-membered heterocyclyl R⁸ groups may be:
iii) monocyclic or polycyclic ring systems wherein the rings in the polycyclic ring systems are fused, bridged or spiro attached; and
iv) in addition to the optional C₀₋₃alkylene-R²⁵ substituents, may also be optionally substituted with one or more oxo groups;
R⁹ is selected from hydrogen, C₁₋₄alkyl, C₁₋₄alkoxy, C₁₋₄haloalkyl, C₁₋₄haloalkoxy, halo, cyano, hydroxy, C₃₋₇cycloalkyl, and S(O)₂C₁₋₄alkyl;
R¹⁰ is selected from hydrogen, C₁₋₄alkyl, C₁₋₄alkoxy, C₁₋₄haloalkyl, halo, cyano, and hydroxy;
R¹¹ and R¹² are independently selected from hydrogen, C₁₋₄alkyl, and halo;
R¹³, R¹⁴, R¹⁵, R¹⁶, and R¹⁷ are independently selected from hydrogen and C₁₋₄alkyl;
R¹⁸ and R¹⁹ are independently selected from hydrogen, C₁₋₄alkyl, C₃₋₇cycloalkyl, 4- to 7-membered heterocyclyl, and 5- or 6-membered heteroaryl, wherein said C₁₋₄alkyl, C₃₋₇cycloalkyl, 4- to 7-membered heterocyclyl, and 5- or 6-membered heteroaryl groups are optionally substituted with one or more substituents selected from C₁₋₄alkyl, halo, OH, C₁₋₄alkoxy, C₃₋₇cycloalkyl, NR³⁴R³⁵, C(O)OR³⁶, and C(O)NR³⁷R³⁸;
R²⁰ and R²¹ are independently selected from hydrogen, C₁₋₄alkyl, C₁₋₄haloalkyl, C₃₋₇cycloalkyl, C(O)C₁₋₄alkyl, C(O)NR³⁹R⁴⁰, and S(O)₂R⁴¹;
R²² is selected from hydroxy, C₁₋₄alkoxy, and NR⁴²R⁴³;
R²³ and R²⁴ are independently selected from hydrogen and C₁₋₄alkyl;
R²⁶ and R²⁷ are independently selected from hydrogen, C₁₋₄alkyl, C₁₋₄haloalkyl, and C₃₋₇cycloalkyl;
R²⁸ is hydrogen or C₁₋₄alkyl;
R²⁹ is C₁₋₄alkyl, C₁₋₄haloalkyl, or NR⁴⁴R⁴⁵;
R³⁰ is selected from C₁₋₄alkyl, hydroxy, C₁₋₄alkoxy, NR⁴⁶R⁴⁷, and 4- to 6-membered heterocyclyl;
R³¹ is C₁₋₄alkyl or NR⁴⁸R⁴⁹; and
R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, and R⁴⁹ are independently selected from hydrogen and C₁₋₄alkyl; or
R³⁴ and R³⁵; R³⁷ and R³⁸; R³⁹ and R⁴⁰; R⁴² and R⁴³; R⁴⁴ and R⁴⁵; R⁴⁶ and R⁴⁷; or R⁴⁸ and R⁴⁹, together with the respective nitrogen atom to which they are attached, form a 4- to 7-membered heterocyclic ring.

2. A compound according to claim 1, wherein the compound has a structure according to formula IA: 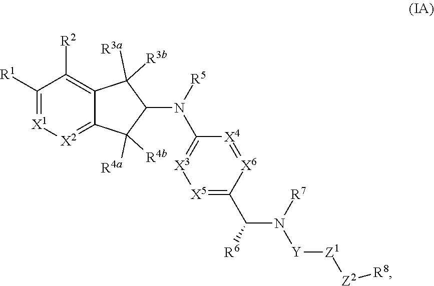
or a pharmaceutically acceptable salt thereof.

3. A compound according to claim 1 or claim 2, wherein:
a. one of X¹ and X² is CH and the other is N;
b. both of X¹ and X² are CR⁹; or
c. both of X¹ and X² are CH.

4. A compound according to any one of claims 1 to 3, wherein;
a. X³, X⁴, X⁵, and X⁶ are independently selected from CH or N, provided that no more than two of X³, X⁴, X⁵, and X⁶ are N;
b. one of X³, X⁴, X⁵, and X⁶ is N and the other three are CH; or
c. X³, X⁴, X⁵, and X⁶ are all CH.

5. A compound according to any one of claims 1 to 4, wherein Y is

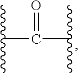 [SMILES: *C(*)=O]

6. A compound according to any one of claims 1 to 5, wherein Z¹ is absent.

7. A compound according to any one of claims 1 to 6, wherein Z² is absent or C₁₋₃alkylene.

8. A compound according to any one of claims 1 to 7, wherein R¹ and R² are independently selected from:
a. hydrogen, C₁₋₂alkyl, C₁₋₄alkoxy, C₁₋₂haloalkyl, halo, and cyano; or
b. hydrogen, methyl, ethyl, methoxy, trifluoromethyl, fluoro, chloro and cyano.

9. A compound according to any one of claims 1 to 8, wherein R^{3a}, R^{3b}, R^{4a}, and R^{4b} are independently selected from hydrogen, C₁₋₄alkyl, and OR¹⁸.

10. A compound according to claim 9, wherein R^{3a} and R^{3b} are both C₁₋₄alkyl, such as both methyl.

11. A compound according to any one of claims 1 to 10, wherein R⁵ is hydrogen.

12. A compound according to any one of claims 1 to 11, wherein R⁶ is C₁₋₃haloalkyl, such as trifluoromethyl.

13. A compound according to any one of claims 1 to 12, wherein R⁷ is C₁₋₃alkyl, such as methyl.

14. A compound according to any one of claims 1 to 13, wherein R⁸ is selected from OR¹⁹, NR²⁰R²¹, C(O)R²², S(O)₂NR²³R²⁴, cyano, C₃₋₈cycloalkyl, 4- to 10-membered heterocyclyl, and 5- to 6-membered heteroaryl, wherein said C₃₋₈cycloalkyl, 4- to 10-membered heterocyclyl, and 5- to 6-membered heteroaryl groups are optionally substituted with one or more substituents of formula C₀₋₃alkylene-R²⁵, wherein each R²⁵ is independently selected from C₁₋₄alkyl, hydroxy, C₁₋₄alkoxy, halo, C₁₋₄haloalkyl, C₃₋₆cycloalkyl, 4- to 6-membered heterocyclyl, NR²⁶R²⁷, NR²⁸C(O)R²⁹, C(O)R³⁰, S(O)₂R³¹ and NR³²S(O)₂R³³; and
wherein said C₃₋₈cycloalkyl and 4- to 10-membered heterocyclyl R⁸ groups may be: monocyclic or polycyclic ring systems wherein the rings in the polycyclic ring systems are fused, bridged or spiro attached; and
in addition to the optional C₀₋₃alkylene-R²⁵ substituents, may also be optionally substituted with one or more oxo groups.

15. A compound according to claim 1, wherein the compound is selected from one of the following compounds, or a pharmaceutically acceptable salt thereof:
N—((S)-1-(4-((-1,1-dimethyl-2,3-dihydro-1H-inden-2-yl)amino)phenyl)-2,2,2-trifluoroethyl)-N-methyltetrahydro-2H-thiopyran-4-carboxamide 1,1-dioxide;
(S)—N-(1-(4-((2,3-dihydro-1H-inden-2-yl)amino)phenyl)-2,2,2-trifluoroethyl)-N-methyltetrahydro-2H-thiopyran-4-carboxamide 1,1-dioxide;
N-((1S)-1-(4-((1,1-dimethyl-2,3-dihydro-1H-inden-2-yl)amino)phenyl)-2,2,2-trifluoroethyl)-N-methyloxazole-5-carboxamide;
1-acetyl-N-((1S)-1-(4-((1,1-dimethyl-2,3-dihydro-1H-inden-2-yl)amino)phenyl)-2,2,2-trifluoroethyl)-N-methylazetidine-3-carboxamide;
N-Methyl-N—((S)-2,2,2-trifluoro-1-(4-(((1,2-cis)-1-methyl-2,3-dihydro-1H-inden-2-yl)amino)phenyl)ethyl)tetrahydro-2H-thiopyran-4-carboxamide 1,1-dioxide;
N-Methyl-N—((S)-2,2,2-trifluoro-1-(4-(((1,2-trans)-1-methyl-2,3-dihydro-1H-inden-2-yl)amino)phenyl)ethyl)tetrahydro-2H-thiopyran-4-carboxamide 1,1-dioxide;
N-methyl-N-((1S)-2,2,2-trifluoro-1-(4-((1-methoxy-2,3-dihydro-1H-inden-2-yl)amino)phenyl)ethyl)tetrahydro-2H-thiopyran-4-carboxamide 1,1-dioxide;
N—((S)-1-(4-(((S)-1,1-dimethyl-2,3-dihydro-1H-inden-2-yl)amino)phenyl)-2,2,2-trifluoroethyl)-N-methylmorpholine-4-carboxamide;
N—((S)-1-(4-(((R)-1,1-dimethyl-2,3-dihydro-1H-inden-2-yl)amino)phenyl)-2,2,2-trifluoroethyl)-N-methylmorpholine-4-carboxamide;
N-((1S)-1-(4-((1,1-dimethyl-2,3-dihydro-1H-inden-2-yl)amino)phenyl)-2,2,2-trifluoroethyl)-N-methylthiomorpholine-4-carboxamide 1,1-dioxide;
N-((1S)-1-(4-((1,1-dimethyl-2,3-dihydro-1H-inden-2-yl)amino)phenyl)-2,2,2-trifluoroethyl)-4-hydroxy-N,4-dimethylpiperidine-1-carboxamide;
N-((1S)-1-(4-((1,1-dimethyl-2,3-dihydro-1H-inden-2-yl)amino)phenyl)-2,2,2-trifluoroethyl)-N-methyltetrahydro-2H-pyran-4-carboxamide;
N-((1S)-1-(4-((1,1-dimethyl-2,3-dihydro-1H-inden-2-yl)amino)phenyl)-2,2,2-trifluoroethyl)-N-methyl-2-oxo-1,2-dihydropyridine-4-carboxamide;
N-((1S)-1-(4-((1,1-dimethyl-2,3-dihydro-1H-inden-2-yl)amino)phenyl)-2,2,2-trifluoroethyl)-4-hydroxy-N-methylpiperidine-1-carboxamide;
3-acetamido-N-((1S)-1-(4-((1,1-dimethyl-2,3-dihydro-1H-inden-2-yl)amino)phenyl)-2,2,2-trifluoroethyl)-N-methylpropanamide;
N-((1S)-1-(4-((1,1-dimethyl-2,3-dihydro-1H-inden-2-yl)amino)phenyl)-2,2,2-trifluoroethyl)-N-methyloxazole-4-carboxamide;
N-((1S)-1-(4-((1,1-dimethyl-2,3-dihydro-1H-inden-2-yl)amino)phenyl)-2,2,2-trifluoroethyl)-2-methoxy-N-methylacetamide;
N-((1S)-1-(4-((1,1-dimethyl-2,3-dihydro-1H-inden-2-yl)amino)phenyl)-2,2,2-trifluoroethyl)-2-hydroxy-N-methylacetamide;
N-((1S)-1-(4-((1,1-dimethyl-2,3-dihydro-1H-inden-2-yl)amino)phenyl)-2,2,2-trifluoroethyl)-3-methoxy-N-methylpropanamide;
N-((1S)-1-(4-((1,1-dimethyl-2,3-dihydro-1H-inden-2-yl)amino)phenyl)-2,2,2-trifluoroethyl)-3-hydroxy-N-methylpropanamide;
N-((1S)-1-(6-((1,1-dimethyl-2,3-dihydro-1H-inden-2-yl)amino)pyridin-3-yl)-2,2,2-trifluoroethyl)-N-methyltetrahydro-2H-thiopyran-4-carboxamide 1,1-dioxide;
N-((1S)-1-(4-((1,3-dimethyl-2,3-dihydro-1H-inden-2-yl)amino)phenyl)-2,2,2-trifluoroethyl)-N-methyltetrahydro-2H-thiopyran-4-carboxamide 1,1-dioxide;
2-acetamido-N-((1S)-1-(4-((1,1-dimethyl-2,3-dihydro-1H-inden-2-yl)amino)phenyl)-2,2,2-trifluoroethyl)-N-methylacetamide;
1-acetyl-N-((1S)-1-(4-((1,1-dimethyl-2,3-dihydro-1H-inden-2-yl)amino)phenyl)-2,2,2-trifluoroethyl)-N,3-dimethylazetidine-3-carboxamide;
1-acetyl-N-((1S)-1-(4-((1,1-dimethyl-2,3-dihydro-1H-inden-2-yl)amino)phenyl)-2,2,2-trifluoroethyl)-3-fluoro-N-methylazetidine-3-carboxamide;
N-((1S)-1-(4-((1,1-dimethyl-2,3-dihydro-1H-inden-2-yl)amino)phenyl)-2,2,2-trifluoroethyl)-N-methyl-2-(2-oxopyrrolidin-1-yl)acetamide;
N-((1S)-1-(4-((1,1-dimethyl-2,3-dihydro-1H-inden-2-yl)amino)phenyl)-2,2,2-trifluoroethyl)-N-methyl-3-(2-oxopyrrolidin-1-yl)propenamide;
5-(((1S)-1-(4-((1,1-dimethyl-2,3-dihydro-1H-inden-2-yl)amino)phenyl)-2,2,2-trifluoroethyl)(methyl)amino)-5-oxopentanoic acid;
N-((1S)-1-(4-((1,1-dimethyl-2,3-dihydro-1H-inden-2-yl)amino)phenyl)-2,2,2-trifluoroethyl)-N-methyl-1,2,5-oxadiazole-3-carboxamide;
4-(((1S)-1-(4-((1,1-dimethyl-2,3-dihydro-1H-inden-2-yl)amino)phenyl)-2,2,2-trifluoroethyl)(methyl)amino)-4-oxobutanoic acid;
N-methyl-N-((1S)-2,2,2-trifluoro-1-(4-((5-methoxy-2,3-dihydro-1H-inden-2-yl)amino)phenyl)ethyl)tetrahydro-2H-thiopyran-4-carboxamide 1,1-dioxide;
N-((1S)-1-(4-((5-chloro-2,3-dihydro-1H-inden-2-yl)amino)phenyl)-2,2,2-trifluoroethyl)-N-methyltetrahydro-2H-thiopyran-4-carboxamide 1,1-dioxide;
1-acetylazetidin-3-yl ((1S)-1-(4-((1,1-dimethyl-2,3-dihydro-1H-inden-2-yl)amino)phenyl)-2,2,2-trifluoroethyl)(methyl)carbamate;
tert-butyl 3-((((1S)-1-(4-((1,1-dimethyl-2,3-dihydro-1H-inden-2-yl)amino)phenyl)-2,2,2-trifluoroethyl)(methyl)carbamoyl)oxy)azetidine-1-carboxylate;
azetidin-3-yl ((1S)-1-(4-((1,1-dimethyl-2,3-dihydro-1H-inden-2-yl)amino)phenyl)-2,2,2-trifluoroethyl)(methyl)carbamate hydrochloride;
N-((1S)-1-(4-((1,1-dimethyl-2,3-dihydro-1H-inden-2-yl)amino)phenyl)-2,2,2-trifluoroethyl)-N-methyl-3-(N-methylsulfamoyl)propenamide;
N-((1R)-1-(4-((1,1-dimethyl-2,3-dihydro-1H-inden-2-yl)amino)phenyl)ethyl)-N-methyltetrahydro-2H-thiopyran-4-carboxamide 1,1-dioxide;
N-((1S)-1-(4-((1,1-dimethyl-2,3-dihydro-1H-inden-2-yl)amino)phenyl)-2,2,2-trifluoroethyl)-N-methyl-4-(N-methylsulfamoyl)butanamide;
N-((1S)-1-(4-((1,1-dimethyl-2,3-dihydro-1H-inden-2-yl)amino)phenyl)-2,2,2-trifluoroethyl)-2-(2,4-dioxoimidazolidin-1-yl)-N-methylacetamide;
N-((1S)-1-(4-((1,1-dimethyl-2,3-dihydro-1H-inden-2-yl)amino)phenyl)-2,2,2-trifluoroethyl)-N-methyl-1-(methylsulfonyl)azetidine-3-carboxamide;
(S)—N-(1-(4-((2,3-dihydro-1H-inden-2-yl)amino)phenyl)-2,2,2-trifluoroethyl)-N-methylpiperidine-4-carboxamide;
(S)—N-(1-(5-((2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(4-((1,1-dimethyl-2,3-dihydro-1H-inden-2-yl)amino)phenyl)-2,2,2-trifluoroethyl)-N-methylazetidine-3-carboxamide;
N3-((1S)-1-(4-((1,1-dimethyl-2,3-dihydro-1H-inden-2-yl)amino)phenyl)-2,2,2-trifluoroethyl)-N1,N3-dimethylazetidine-1,3-dicarboxamide;
N-((1S)-1-(4-((1,1-dimethyl-2,3-dihydro-1H-inden-2-yl)amino)phenyl)-2,2,2-trifluoroethyl)-N,1-dimethylazetidine-3-carboxamide;
N-((1S)-1-(5-((1,1-dimethyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(4-((1,1-dimethyl-2,3-dihydro-1H-inden-2-yl)amino)phenyl)-2,2,2-trifluoroethyl)-N-methylcyclopropanesulfonamide;
N-((1S)-1-(4-((1,1-dimethyl-2,3-dihydro-1H-inden-2-yl)amino)phenyl)-2,2,2-trifluoroethyl)tetrahydro-2H-thiopyran-4-carboxamide 1,1-dioxide;
N-(1-(5-((1,1-dimethyl-2,3-dihydro-1H-inden-2-yl)amino)pyridin-2-yl)-2,2,2-trifluoroethyl)-N-methylazetidine-3-carboxamide;
N-(1-(6-((1,1-dimethyl-2,3-dihydro-1H-inden-2-yl)amino)pyridin-3-yl)-2,2,2-trifluoroethyl)-N-methylazetidine-3-carboxamide;
N-(1-(5-((1,1-dimethyl-2,3-dihydro-1H-inden-2-yl)amino)pyridin-2-yl)-2,2,2-trifluoroethyl)-N,1-dimethylazetidine-3-carboxamide;
N-(1-(6-((1,1-dimethyl-2,3-dihydro-1H-inden-2-yl)amino)pyridin-3-yl)-2,2,2-trifluoroethyl)-N,1-dimethylazetidine-3-carboxamide;
N-(1-(5-((1,1-dimethyl-2,3-dihydro-1H-inden-2-yl)amino)pyridin-2-yl)-2,2,2-trifluoroethyl)-N-methyl-2-oxo-1,2-dihydropyridine-4-carboxamide;
N-(1-(6-((1,1-dimethyl-2,3-dihydro-1H-inden-2-yl)amino)pyridin-3-yl)-2,2,2-trifluoroethyl)-N-methyl-2-oxo-1,2-dihydropyridine-4-carboxamide;
N-((1S)-1-(5-((1,1-dimethyl-2,3-dihydro-1H-inden-2-yl)amino)pyridin-2-yl)-2,2,2-trifluoroethyl)-N-methyl-6-oxopiperidine-3-carboxamide;
N-((1S)-1-(5-((1,1-dimethyl-2,3-dihydro-1H-inden-2-yl)amino)pyridin-2-yl)-2,2,2-trifluoroethyl)-N-methyl-2-oxooxazolidine-5-carboxamide;
N-((1S)-1-(5-((1,1-dimethyl-2,3-dihydro-1H-inden-2-yl)amino)pyridin-2-yl)-2,2,2-trifluoroethyl)-3-(dimethylamino)-N-methylcyclobutane-1-carboxamide;
N-((1S)-1-(6-((1,1-Dimethyl-2,3-dihydro-1H-inden-2-yl)amino)pyridin-3-yl)-2,2,2-trifluoroethyl)-3-trans-(dimethylamino)-N-methylcyclobutane-1-carboxamide;
N-((1S)-1-(5-((1,1-dimethyl-2,3-dihydro-1H-inden-2-yl)amino)pyridin-2-yl)-2,2,2-trifluoroethyl)-N-methyl-2-oxopiperidine-4-carboxamide;
N-((1S)-1-(6-((1,1-dimethyl-2,3-dihydro-1H-inden-2-yl)amino)pyridin-3-yl)-2,2,2-trifluoroethyl)-N-methylquinuclidine-3-carboxamide;
N-((1S)-1-(5-((1,1-dimethyl-2,3-dihydro-1H-inden-2-yl)amino)pyridin-2-yl)-2,2,2-trifluoroethyl)-N-methylpiperidine-3-carboxamide;
N-((1S)-1-(5-((1,1-dimethyl-2,3-dihydro-1H-inden-2-yl)amino)pyridin-2-yl)-2,2,2-trifluoroethyl)-N,1-dimethylpiperidine-3-carboxamide;
N-((1S)-1-(5-((1,1-dimethyl-2,3-dihydro-1H-inden-2-yl)amino)pyridin-2-yl)-2,2,2-trifluoroethyl)-N,1-dimethylpiperidine-4-carboxamide;
N-((1S)-1-(5-((1,1-dimethyl-2,3-dihydro-1H-inden-2-yl)amino)pyridin-2-yl)-2,2,2-trifluoroethyl)-N-methyl-5-oxomorpholine-2-carboxamide;
N-((1S)-1-(5-((1,1-dimethyl-2,3-dihydro-1H-inden-2-yl)amino)pyridin-2-yl)-2,2,2-trifluoroethyl)-N-methylquinuclidine-3-carboxamide;
N-((1S)-1-(5-((1,1-dimethyl-2,3-dihydro-1H-inden-2-yl)amino)pyridin-2-yl)-2,2,2-trifluoroethyl)-N-methylthiomorpholine-4-carboxamide 1,1-dioxide;
N-((1S)-1-(6-((1,1-dimethyl-2,3-dihydro-1H-inden-2-yl)amino)pyridin-3-yl)-2,2,2-trifluoroethyl)-N,1-dimethylpiperidine-4-carboxamide;
N-((1S)-1-(5-((1,1-dimethyl-2,3-dihydro-1H-inden-2-yl)amino)pyridin-2-yl)-2,2,2-trifluoroethyl)-N-methylpiperidine-4-carboxamide;
N-((1S)-1-(6-((1,1-dimethyl-2,3-dihydro-1H-inden-2-yl)amino)pyridin-3-yl)-2,2,2-trifluoroethyl)-N-methylpiperidine-4-carboxamide;
N-((1S)-1-(4-((6-chloro-1,1-dimethyl-2,3-dihydro-1H-inden-2-yl)amino)phenyl)-2,2,2-trifluoroethyl)-N-methyltetrahydro-2H-thiopyran-4-carboxamide 1,1-dioxide;
N-((1S)-1-(4-((5-chloro-1,1-dimethyl-2,3-dihydro-1H-inden-2-yl)amino)phenyl)-2,2,2-trifluoroethyl)-N-methyltetrahydro-2H-thiopyran-4-carboxamide 1,1-dioxide;
N-((1S)-1-(5-((1,1-dimethyl-2,3-dihydro-1H-inden-2-yl)amino)pyridin-2-yl)-2,2,2-trifluoroethyl)-N-methyl-1-azaspiro[3.3]heptane-6-carboxamide;
(4,6-trans)-N-((1S)-1-(5-((1,1-dimethyl-2,3-dihydro-1H-inden-2-yl)amino)pyridin-2-yl)-2,2,2-trifluoroethyl)-N,1-dimethyl-1-azaspiro[3.3]heptane-6-carboxamide;
(4,6-cis)-N-((1S)-1-(5-((1,1-dimethyl-2,3-dihydro-1H-inden-2-yl)amino)pyridin-2-yl)-2,2,2-trifluoroethyl)-N,1-dimethyl-1-azaspiro[3.3]heptane-6-carboxamide;
(S)—N—((S)-1-(5-(((S)-1,1-dimethyl-2,3-dihydro-1H-inden-2-yl)amino)pyridin-2-yl)-2,2,2-trifluoroethyl)-N-methyl-5-oxopyrrolidine-3-carboxamide;
N-((1S)-1-(5-((1,1-dimethyl-2,3-dihydro-1H-inden-2-yl)amino)pyridin-2-yl)-2,2,2-trifluoroethyl)-N-methyl-5-oxomorpholine-3-carboxamide;
(S)—N—((S)-1-(5-(((S)-1,1-dimethyl-2,3-dihydro-1H-inden-2-yl)amino)pyridin-2-yl)-2,2,2-trifluoroethyl)-N-methyl-4-oxoazetidine-2-carboxamide;
(R)—N—((S)-1-(5-(((S)-1,1-dimethyl-2,3-dihydro-1H-inden-2-yl)amino)pyridin-2-yl)-2,2,2-trifluoroethyl)-N-methyl-5-oxopyrrolidine-3-carboxamide;
(S)—N—((S)-1-(5-(((S)-1,1-dimethyl-2,3-dihydro-1H-inden-2-yl)amino)pyridin-2-yl)-2,2,2-trifluoroethyl)-N-methylmorpholine-2-carboxamide dihydrochloride;
(S)—N—((S)-1-(5-(((S)-1,1-dimethyl-2,3-dihydro-1H-inden-2-yl)amino)pyridin-2-yl)-2,2,2-trifluoroethyl)-N-methylpyrrolidine-3-carboxamide;
(S)-1-acetyl-N—((S)-1-(5-(((S)-1,1-dimethyl-2,3-dihydro-1H-inden-2-yl)amino)pyridin-2-yl)-2,2,2-trifluoroethyl)-N-methylpyrrolidine-3-carboxamide;
(S)—N—((S)-1-(5-(((S)-1,1-dimethyl-2,3-dihydro-1H-inden-2-yl)amino)pyridin-2-yl)-2,2,2-trifluoroethyl)-N-methyl-5-oxopyrrolidine-2-carboxamide;
(R)-1-acetyl-N—((S)-1-(5-(((S)-1,1-dimethyl-2,3-dihydro-1H-inden-2-yl)amino)pyridin-2-yl)-2,2,2-trifluoroethyl)-N-methylpyrrolidine-2-carboxamide;
(1R,3S)-3-acetamido-N—((S)-1-(5-(((S)-1,1-dimethyl-2,3-dihydro-1H-inden-2-yl)amino)pyridin-2-yl)-2,2,2-trifluoroethyl)-N-methylcyclopentane-1-carboxamide;
N—((S)-1-(5-(((S)-1,1-dimethyl-2,3-dihydro-1H-inden-2-yl)amino)pyridin-2-yl)-2,2,2-trifluoroethyl)-N-methyl-2-azaspiro[3.3]heptane-6-carboxamide;
N-((1S)-1-(6-((1,1-dimethyl-2,3-dihydro-1H-inden-2-yl)amino)pyridin-3-yl)-2,2,2-trifluoroethyl)-N-methyl-7-oxo-6-oxa-8-azaspiro[3.5]nonane-2-carboxamide;
(R)—N—((S)-1-(5-(((S)-1,1-dimethyl-2,3-dihydro-1H-inden-2-yl)amino)pyridin-2-yl)-2,2,2-trifluoroethyl)-N-methylpyrrolidine-3-carboxamide;
N-methyl-N-((1S)-2,2,2-trifluoro-1-(5-((4-methoxy-2,3-dihydro-1H-inden-2-yl)amino)pyridin-2-yl)ethyl)tetrahydro-2H-thiopyran-4-carboxamide 1,1-dioxide;
3-acetamido-N—((S)-1-(5-(((S)-1,1-dimethyl-2,3-dihydro-1H-inden-2-yl)amino)pyridin-2-yl)-2,2,2-trifluoroethyl)-N-methylbicyclo[1.1.1]pentane-1-carboxamide;
(R)—N—((S)-1-(5-(((S)-1,1-dimethyl-2,3-dihydro-1H-inden-2-yl)amino)pyridin-2-yl)-2,2,2-trifluoroethyl)-N,4-dimethylmorpholine-2-carboxamide;
(S)-4-acetyl-N—((S)-1-(5-(((S)-1,1-dimethyl-2,3-dihydro-1H-inden-2-yl)amino)pyridin-2-yl)-2,2,2-trifluoroethyl)-N-methylmorpholine-2-carboxamide;
(1,3-cis)-3-acetamido-N—((S)-1-(5-(((S)-1,1-dimethyl-2,3-dihydro-1H-inden-2-yl)amino)pyridin-2-yl)-2,2,2-trifluoroethyl)-N-methylcyclobutane-1-carboxamide;
(2S,4R)-1-acetyl-N—((S)-1-(5-(((S)-1,1-dimethyl-2,3-dihydro-1H-inden-2-yl)amino)pyridin-2-yl)-2,2,2-trifluoroethyl)-4-hydroxy-N-methylpyrrolidine-2-carboxamide;
(1R,3S)—N—((S)-1-(5-(((S)-1,1-dimethyl-2,3-dihydro-1H-inden-2-yl)amino)pyridin-2-yl)-2,2,2-trifluoroethyl)-N-methyl-3-(2,2,2-trifluoroacetamido)cyclopentane-1-carboxamide;
N1-((S)-1-(5-(((S)-1,1-dimethyl-2,3-dihydro-1H-inden-2-yl)amino)pyridin-2-yl)-2,2,2-trifluoroethyl)-N1,N3-dimethylbicyclo[1.1.1]pentane-1,3-dicarboxamide;
(1,3-trans)-3-acetamido-N—((S)-1-(5-(((S)-1,1-dimethyl-2,3-dihydro-1H-inden-2-yl)amino)pyridin-2-yl)-2,2,2-trifluoroethyl)-N-methylcyclobutane-1-carboxamide;
(1,3-cis)-3-acetamido-N—((S)-1-(5-(((S)-1,1-dimethyl-2,3-dihydro-1H-inden-2-yl)amino)pyridin-2-yl)-2,2,2-trifluoroethyl)-N-methylcyclopentane-1-carboxamide;
N-((1S)-1-(5-((1,1-dimethyl-2,3-dihydro-1H-inden-2-yl)amino)pyridin-2-yl)-2,2,2-trifluoroethyl)-N-ethyltetrahydro-2H-thiopyran-4-carboxamide 1,1-dioxide;
N-methyl-N-((1S)-2,2,2-trifluoro-1-(5-((4-(trifluoromethyl)-2,3-dihydro-1H-inden-2-yl)amino)pyridin-2-yl)ethyl)tetrahydro-2H-thiopyran-4-carboxamide 1,1-dioxide;
(R)-1-acetyl-N—((S)-1-(5-(((S)-1,1-dimethyl-2,3-dihydro-1H-inden-2-yl)amino)pyridin-2-yl)-2,2,2-trifluoroethyl)-N-methylpyrrolidine-3-carboxamide;
(R)—N—((S)-1-(5-(((S)-1,1-dimethyl-2,3-dihydro-1H-inden-2-yl)amino)pyridin-2-yl)-2,2,2-trifluoroethyl)-N-methylmorpholine-2-carboxamide;
(S)-1-acetyl-N—((S)-1-(5-(((S)-1,1-dimethyl-2,3-dihydro-1H-inden-2-yl)amino)pyridin-2-yl)-2,2,2-trifluoroethyl)-N-methylpyrrolidine-2-carboxamide;
(S)—N—((S)-1-(5-(((S)-1,1-dimethyl-2,3-dihydro-1H-inden-2-yl)amino)pyridin-2-yl)-2,2,2-trifluoroethyl)-N,4-dimethylmorpholine-2-carboxamide;
N—((S)-1-(5-(((S)-1,1-dimethyl-2,3-dihydro-1H-inden-2-yl)amino)pyridin-2-yl)-2,2,2-trifluoroethyl)-2-(1,1-dioxidotetrahydro-2H-thiopyran-4-yl)-N-methylacetamide;
N-((1S)-1-(5-((4-ethyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N—((S)-1-(5-(((S)-5-chloro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N—((S)-1-(5-(((R)-5-chloro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N—((S)-1-(5-(((S)-1,1-dimethyl-2,3-dihydro-1H-inden-2-yl)amino)pyridin-2-yl)-2,2,2-trifluoroethyl)-N,1-dimethyl-5-oxopiperazine-2-carboxamide;
N—((R)-1-(5-(((R)-1,1-dimethyl-2,3-dihydro-1H-inden-2-yl)amino)pyridin-2-yl)-2,2,2-trifluoroethyl)-N,1-dimethyl-5-oxopiperazine-2-carboxamide;
N1-((S)-1-(5-(((R)-1,1-dimethyl-2,3-dihydro-1H-inden-2-yl)amino)pyridin-2-yl)-2,2,2-trifluoroethyl)-N1,N3-dimethylbicyclo[1.1.1]pentane-1,3-dicarboxamide;
(1R,3S)-3-acetamido-N—((S)-1-(5-(((R)-1,1-dimethyl-2,3-dihydro-1H-inden-2-yl)amino)pyridin-2-yl)-2,2,2-trifluoroethyl)-N-methylcyclopentane-1-carboxamide;
(1,3-cis)-3-acetamido-N—((S)-1-(5-(((R)-1,1-dimethyl-2,3-dihydro-1H-inden-2-yl)amino)pyridin-2-yl)-2,2,2-trifluoroethyl)-N-methylcyclobutane-1-carboxamide;
(1,3-trans)-3-acetamido-N—((S)-1-(5-(((R)-1,1-dimethyl-2,3-dihydro-1H-inden-2-yl)amino)pyridin-2-yl)-2,2,2-trifluoroethyl)-N-methylcyclobutane-1-carboxamide;
(2,4-cis)-N-((1S)-1-(5-((1,1-dimethyl-2,3-dihydro-1H-inden-2-yl)amino)pyridin-2-yl)-2,2,2-trifluoroethyl)-N-methyl-6-oxo-7-oxa-5-azaspiro[3.4]octane-2-carboxamide;
N-((1S)-1-(5-((1,1-dimethyl-2,3-dihydro-1H-inden-2-yl)amino)pyridin-2-yl)-2,2,2-trifluoroethyl)-N-methyl-1-(methylsulfonyl)azetidine-3-carboxamide;
(S)—N—((S)-1-(5-(((R)-1,1-dimethyl-2,3-dihydro-1H-inden-2-yl)amino)pyridin-2-yl)-2,2,2-trifluoroethyl)-N-methyl-5-oxopyrrolidine-3-carboxamide;
N-((1S)-1-(5-((1,1-dimethyl-2,3-dihydro-1H-inden-2-yl)amino)pyridin-2-yl)-2,2,2-trifluoroethyl)-2-(2,4-dioxoimidazolidin-1-yl)-N-methylacetamide;
N-((1S)-1-(5-((1,1-dimethyl-2,3-dihydro-1H-inden-2-yl)amino)pyridin-2-yl)-2,2,2-trifluoroethyl)-N-methyl-3-thiabicyclo[3.1.0]hexane-6-carboxamide 3,3-dioxide;
(R)—N—((S)-1-(6-(((S)-1,1-dimethyl-2,3-dihydro-1H-inden-2-yl)amino)pyridin-3-yl)-2,2,2-trifluoroethyl)-N-methyl-6-oxopiperidine-3-carboxamide;
(S)—N—((S)-1-(6-(((S)-1,1-dimethyl-2,3-dihydro-1H-inden-2-yl)amino)pyridin-3-yl)-2,2,2-trifluoroethyl)-N-methyl-6-oxopiperidine-3-carboxamide;
(2S,4R)-1-acetyl-N—((S)-1-(5-(((R)-1,1-dimethyl-2,3-dihydro-1H-inden-2-yl)amino)pyridin-2-yl)-2,2,2-trifluoroethyl)-4-hydroxy-N-methylpyrrolidine-2-carboxamide;
(1S, 3R)—N—((S)-1-(5-(((S)-1,1-dimethyl-2,3-dihydro-1H-inden-2-yl)amino)pyridin-2-yl)-2,2,2-trifluoroethyl)-N-methyl-3-(methylsulfonamido)cyclopentane-1-carboxamide;
(1,3-trans)-N—((S)-1-(5-(((S)-1,1-dimethyl-2,3-dihydro-1H-inden-2-yl)amino)pyridin-2-yl)-2,2,2-trifluoroethyl)-N-methyl-3-(methylsulfonamido)cyclobutane-1-carboxamide;
N—((S)-1-(5-(((S)-1,1-dimethyl-2,3-dihydro-1H-inden-2-yl)amino)pyridin-2-yl)-2,2,2-trifluoroethyl)-N-methyl-1-(methylsulfonyl)piperidine-4-carboxamide;
1-acetyl-N—((S)-1-(5-(((S)-1,1-dimethyl-2,3-dihydro-1H-inden-2-yl)amino)pyridin-2-yl)-2,2,2-trifluoroethyl)-N-methylpiperidine-4-carboxamide;
N—((S)-1-(5-(((S)-1,1-dimethyl-2,3-dihydro-1H-inden-2-yl)amino)pyridin-2-yl)-2,2,2-trifluoroethyl)-N,1-dimethyl-5-oxopyrrolidine-2-carboxamide;
N³-((1S)-1-(5-((1,1-dimethyl-2,3-dihydro-1H-inden-2-yl)amino)pyridin-2-yl)-2,2,2-trifluoroethyl)-N¹,N³-dimethylazetidine-1,3-dicarboxamide;
N-((1S)-1-(5-((1,1-dimethyl-2,3-dihydro-1H-inden-2-yl)amino)pyridin-2-yl)-2,2,2-trifluoroethyl)-N-methyl-6-oxo-5-azaspiro[3.4]octane-2-carboxamide;
N—((S)-1-(5-(((S)-1,1-dimethyl-2,3-dihydro-1H-inden-2-yl)amino)pyridin-2-yl)-2,2,2-trifluoroethyl)-N-methyl-3-(methylsulfonamido)bicyclo[1.1.1]pentane-1-carboxamide;
N—((S)-1-(5-(((S)-1,1-dimethyl-2,3-dihydro-1H-inden-2-yl)amino)pyridin-2-yl)-2,2,2-trifluoroethyl)-2-(1,1-dioxidothiomorpholino)-N-methylacetamide;
1-acetyl-N-((1S)-1-(5-((1,1-dimethyl-2,3-dihydro-1H-inden-2-yl)amino)pyridin-2-yl)-2,2,2-trifluoroethyl)-N-methyl-1-azaspiro[3.3]heptane-6-carboxamide;
N-((1S)-1-(5-((4-bromo-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N⁶-((1S)-1-(5-((1,1-dimethyl-2,3-dihydro-1H-inden-2-yl)amino)pyridin-2-yl)-2,2,2-trifluoroethyl)-N¹,N⁶-dimethyl-1-azaspiro[3.3]heptane-1,6-dicarboxamide;
(1,3-cis)-N¹—((S)-1-(5-(((S)-1,1-dimethyl-2,3-dihydro-1H-inden-2-yl)amino)pyridin-2-yl)-2,2,2-trifluoroethyl)-N¹,N³-dimethylcyclobutane-1,3-dicarboxamide;
(1,3-cis)-N—((S)-1-(5-(((S)-1,1-dimethyl-2,3-dihydro-1H-inden-2-yl)amino)pyridin-2-yl)-2,2,2-trifluoroethyl)-N-methyl-3-(methylsulfonamido)cyclobutane-1-carboxamide;
N-((1S)-1-(5-((1,1-dimethyl-2,3-dihydro-1H-inden-2-yl)amino)pyridin-2-yl)-2,2,2-trifluoroethyl)-1-(2-methoxyethyl)-N-methyl-1-azaspiro[3.3]heptane-6-carboxamide;
(2S,4R)—N—((S)-1-(5-(((S)-1,1-dimethyl-2,3-dihydro-1H-inden-2-yl)amino)pyridin-2-yl)-2,2,2-trifluoroethyl)-4-hydroxy-N-methyl-1-(methylsulfonyl)pyrrolidine-2-carboxamide;
N-((1S)-1-(5-((1,1-dimethyl-2,3-dihydro-1H-inden-2-yl)amino)pyridin-2-yl)-2,2,2-trifluoroethyl)-N-methyl-2-(2-oxoimidazolidin-1-yl)acetamide;
(1,3-trans)-N¹—((S)-1-(5-(((S)-1,1-dimethyl-2,3-dihydro-1H-inden-2-yl)amino)pyridin-2-yl)-2,2,2-trifluoroethyl)-N¹,N³-dimethylcyclobutane-1,3-dicarboxamide;
N-((1S)-1-(5-((1,1-dimethyl-2,3-dihydro-1H-inden-2-yl)amino)pyridin-2-yl)-2,2,2-trifluoroethyl)-1-(2-hydroxyethyl)-N-methyl-1-azaspiro[3.3]heptane-6-carboxamide;
N—((S)-1-(5-(((S)-1,1-dimethyl-2,3-dihydro-1H-inden-2-yl)amino)pyridin-2-yl)-2,2,2-trifluoroethyl)-N,1-dimethyl-2-oxopiperidine-4-carboxamide;
N—((S)-1-(5-(((S)-1,1-dimethyl-2,3-dihydro-1H-inden-2-yl)amino)pyridin-2-yl)-2,2,2-trifluoroethyl)-N,1-dimethyl-6-oxopiperidine-3-carboxamide;
N-((1S)-1-(5-((4-cyclopropyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-methyl-N-((1S)-2,2,2-trifluoro-1-(5-((5-(trifluoromethyl)-2,3-dihydro-1H-inden-2-yl)amino)pyridin-2-yl)ethyl)tetrahydro-2H-thiopyran-4-carboxamide 1,1-dioxide;
N-((1S)-1-(5-((4-cyano-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-methyl-N-((1S)-2,2,2-trifluoro-1-(5-((5-fluoro-2,3-dihydro-1H-inden-2-yl)amino)pyridin-2-yl)ethyl)tetrahydro-2H-thiopyran-4-carboxamide 1,1-dioxide;
(S)—N-(1-(5-((5,6-dimethyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4,5-difluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4-chloro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4-chloro-5-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
(S)—N-(1-(5-((4,7-dimethyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4,6-difluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((6-chloro-4-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
(S)—N-(1-(5-((5,6-difluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
(S)—N-(1-(5-((4,7-difluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-methyl-N-((1S)-2,2,2-trifluoro-1-(5-((4-fluoro-2,3-dihydro-1H-inden-2-yl)amino)pyridin-2-yl)ethyl)tetrahydro-2H-thiopyran-4-carboxamide 1,1-dioxide;
N-((1S)-1-(5-((5-chloro-4-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4,5-dichloro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-methyl-N-((1S)-2,2,2-trifluoro-1-(6-((4-(trifluoromethyl)-2,3-dihydro-1H-inden-2-yl)amino)pyridin-3-yl)ethyl)tetrahydro-2H-thiopyran-4-carboxamide 1,1-dioxide;
N-((1S)-1-(5-((4,6-dichloro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((5-bromo-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4-chloro-6-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(6-((5-chloro-2,3-dihydro-1H-inden-2-yl)amino)pyridin-3-yl)-2,2,2-trifluoroethyl)-N-methyltetrahydro-2H-thiopyran-4-carboxamide 1,1-dioxide;
N-((1S)-1-(5-((5-chloro-4-fluoro-2,3-dihydro-1H-inden-2-yl)amino)pyridin-2-yl)-2,2,2-trifluoroethyl)-N-methyl-1-(methylsulfonamido)cyclopropane-1-carboxamide;
N-((1S)-1-(5-((5-chloro-6-(difluoromethoxy)-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N¹-((1S)-1-(5-((5-chloro-2,3-dihydro-1H-inden-2-yl)amino)pyridin-2-yl)-2,2,2-trifluoroethyl)-N¹,N⁴-dimethylcubane-1,4-dicarboxamide;
N-((1S)-1-(5-((4-chloro-7-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-methyl-N-((1S)-2,2,2-trifluoro-1-(5-((4,5,6-trifluoro-2,3-dihydro-1H-inden-2-yl)amino)pyridin-2-yl)ethyl)tetrahydro-2H-thiopyran-4-carboxamide 1,1-dioxide;
(S)—N—((S)-1-(5-(((S)-5-chloro-2,3-dihydro-1H-inden-2-yl)amino)pyridin-2-yl)-2,2,2-trifluoroethyl)-N-methyl-6-oxopiperidine-3-carboxamide;
(S)—N-(1-(5-((5,6-dichloro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N—((S)-1-(5-(((S)-5-chloro-2,3-dihydro-1H-inden-2-yl)amino)pyridin-2-yl)-2,2,2-trifluoroethyl)-N-methyl-3-(methylsulfonamido)bicyclo[1.1.1]pentane-1-carboxamide;
(S)—N—((S)-1-(5-(((S)-5-chloro-2,3-dihydro-1H-inden-2-yl)amino)pyridin-2-yl)-2,2,2-trifluoroethyl)-N-methyl-5-oxopyrrolidine-3-carboxamide;
(R)—N—((S)-1-(5-(((S)-5-chloro-2,3-dihydro-1H-inden-2-yl)amino)pyridin-2-yl)-2,2,2-trifluoroethyl)-N,3-dimethyl-2-oxoimidazolidine-4-carboxamide;
(S)—N-(1-(5-((4,7-dichloro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N—((S)-1-(5-(((S)-5-chloro-2,3-dihydro-1H-inden-2-yl)amino)pyridin-2-yl)-2,2,2-trifluoroethyl)-N-methyl-2-oxooxazolidine-5-carboxamide;
(R)—N—((S)-1-(5-(((S)-5-chloro-2,3-dihydro-1H-inden-2-yl)amino)pyridin-2-yl)-2,2,2-trifluoroethyl)-N-methyl-5-oxopyrrolidine-2-carboxamide hydrochloride;
N-((1S)-1-(5-((5-chloro-4-cyclopropyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((5-cyclopropyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
(R)—N—((S)-1-(5-(((S)-5-chloro-2,3-dihydro-1H-inden-2-yl)amino)pyridin-2-yl)-2,2,2-trifluoroethyl)-N-methyl-6-oxopiperidine-2-carboxamide;
N-((1S)-1-(5-((5-bromo-6-fluoro-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((5-chloro-4-(trifluoromethyl)-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N—((S)-1-(5-(((S)-5-chloro-2,3-dihydro-1H-inden-2-yl)amino)pyridin-2-yl)-2,2,2-trifluoroethyl)-N-methyl-2-oxopiperidine-4-carboxamide;
(S)—N—((S)-1-(5-(((S)-5-chloro-2,3-dihydro-1H-inden-2-yl)amino)pyridin-2-yl)-2,2,2-trifluoroethyl)-N-methyl-5-oxopyrrolidine-2-carboxamide;
(3S)—N-((1S)-1-(5-((5-chloro-4-fluoro-2,3-dihydro-1H-inden-2-yl)amino)pyridin-2-yl)-2,2,2-trifluoroethyl)-N-methyl-5-oxopyrrolidine-3-carboxamide;
(3S)—N-((1S)-1-(5-((4,5-dichloro-2,3-dihydro-1H-inden-2-yl)amino)pyridin-2-yl)-2,2,2-trifluoroethyl)-N-methyl-5-oxopyrrolidine-3-carboxamide;
(3S)—N-((1S)-1-(5-((4,5-dichloro-2,3-dihydro-1H-inden-2-yl)amino)pyridin-2-yl)-2,2,2-trifluoroethyl)-N-methyl-6-oxopiperidine-3-carboxamide;
N-((1S)-1-(5-((4,5-dichloro-2,3-dihydro-1H-inden-2-yl)amino)pyridin-2-yl)-2,2,2-trifluoroethyl)-N-methyl-3-(methylsulfonamido)bicyclo[1.1.1]pentane-1-carboxamide;
N-((1S)-1-(5-((5-(difluoromethoxy)-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4,5-dichloro-2,3-dihydro-1H-inden-2-yl)amino)pyridin-2-yl)-2,2,2-trifluoroethyl)-N-methyl-1-(methylsulfonyl)piperidine-4-carboxamide;
(3S)—N-((1S)-1-(5-((5-chloro-4-cyclopropyl-2,3-dihydro-1H-inden-2-yl)amino)pyridin-2-yl)-2,2,2-trifluoroethyl)-N-methyl-5-oxopyrrolidine-3-carboxamide;
(3S)—N-((1S)-1-(5-((5-chloro-4-cyclopropyl-2,3-dihydro-1H-inden-2-yl)amino)pyridin-2-yl)-2,2,2-trifluoroethyl)-N-methyl-6-oxopiperidine-3-carboxamide;
N-((1S)-1-(6-((5-chloro-4-fluoro-2,3-dihydro-1H-inden-2-yl)amino)pyridin-3-yl)-2,2,2-trifluoroethyl)-N-methyltetrahydro-2H-thiopyran-4-carboxamide 1,1-dioxide;
N-((1S)-1-(5-((4-(difluoromethoxy)-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N³-((1S)-1-(5-((4,5-dichloro-2,3-dihydro-1H-inden-2-yl)amino)pyridin-2-yl)-2,2,2-trifluoroethyl)-N¹,N³-dimethylazetidine-1,3-dicarboxamide;
(3S)—N-((1S)-1-(5-((5-chloro-4-fluoro-2,3-dihydro-1H-inden-2-yl)amino)pyridin-2-yl)-2,2,2-trifluoroethyl)-N-methyl-6-oxopiperidine-3-carboxamide;
(3R)—N-((1S)-1-(6-((5-chloro-4-fluoro-2,3-dihydro-1H-inden-2-yl)amino)pyridin-3-yl)-2,2,2-trifluoroethyl)-N-methyl-5-oxopyrrolidine-3-carboxamide;
N-((1S)-1-(5-((4,5-dichloro-2,3-dihydro-1H-inden-2-yl)amino)pyridin-2-yl)-2,2,2-trifluoroethyl)-N-methyl-6-oxo-5-azaspiro[3.4]octane-2-carboxamide;
N-((1S)-1-(6-((4,5-dichloro-2,3-dihydro-1H-inden-2-yl)amino)pyridin-3-yl)-2,2,2-trifluoroethyl)-N-methyltetrahydro-2H-thiopyran-4-carboxamide 1,1-dioxide;
(3R)—N-((1S)-1-(6-((4,5-dichloro-2,3-dihydro-1H-inden-2-yl)amino)pyridin-3-yl)-2,2,2-trifluoroethyl)-N-methyl-5-oxopyrrolidine-3-carboxamide;
N-((1S)-1-(5-((5-chloro-4-fluoro-2,3-dihydro-1H-inden-2-yl)amino)pyridin-2-yl)-2,2,2-trifluoroethyl)-N,2-dimethylpyrimidine-5-carboxamide;
2-cyano-N-((1S)-1-(5-((4,5-dichloro-2,3-dihydro-1H-inden-2-yl)amino)pyridin-2-yl)-2,2,2-trifluoroethyl)-N-methylacetamide;
N-((1S)-1-(5-((4,5-dichloro-2,3-dihydro-1H-inden-2-yl)amino)pyridin-2-yl)-2,2,2-trifluoroethyl)-N-methyl-1-(tetrahydrofuran-3-carbonyl)azetidine-3-carboxamide;
N-((1S)-1-(5-((4,5-dichloro-2,3-dihydro-1H-inden-2-yl)amino)pyridin-2-yl)-2,2,2-trifluoroethyl)-N-methylthietane-3-carboxamide 1,1-dioxide;
N-((1S)-1-(5-((5-chloro-4-(methoxymethyl)-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((4,5-dichloro-2,3-dihydro-1H-inden-2-yl)amino)pyridin-2-yl)-2,2,2-trifluoroethyl)-N-methyltetrahydrothiophene-3-carboxamide 1,1-dioxide;
N-((1S)-1-(5-((5-chloro-4-fluoro-2,3-dihydro-1H-inden-2-yl)amino)pyridin-2-yl)-2,2,2-trifluoroethyl)-2-(1,1-dioxidoisothiazolidin-2-yl)-N-methylacetamide;
(1,3-trans)-N¹-((1S)-1-(5-((4,5-dichloro-2,3-dihydro-1H-inden-2-yl)amino)pyridin-2-yl)-2,2,2-trifluoroethyl)-N¹,N³-dimethylcyclobutane-1,3-dicarboxamide;
N-((1S)-1-(5-((5-chloro-4-fluoro-2,3-dihydro-1H-inden-2-yl)amino)pyridin-2-yl)-2,2,2-trifluoroethyl)-N-methyl-1-(5-methyl-1,3,4-oxadiazol-2-yl)azetidine-3-carboxamide;
N-((1S)-1-(5-((5-chloro-4-fluoro-2,3-dihydro-1H-inden-2-yl)amino)pyridin-2-yl)-2,2,2-trifluoroethyl)-2-hydroxy-N-methylacetamide;
N-((1S)-1-(5-((5-chloro-4-fluoro-2,3-dihydro-1H-inden-2-yl)amino)pyridin-2-yl)-2,2,2-trifluoroethyl)-N,N′-dimethylcyclopropane-1,1-dicarboxamide;
N-((1S)-1-(5-((5-chloro-4-isopropyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
N-((1S)-1-(5-((5-chloro-4-fluoro-2,3-dihydro-1H-inden-2-yl)amino)pyridin-2-yl)-2,2,2-trifluoroethyl)-N-methyl-1-(oxetane-3-carbonyl)azetidine-3-carboxamide;
N-((1S)-1-(5-((5-chloro-4-methyl-2,3-dihydro-1H-inden-2-yl)amino)pyridin-2-yl)-2,2,2-trifluoroethyl)-N-methyltetrahydro-2H-thiopyran-4-carboxamide 1,1-dioxide;
(1,3-trans)-N-((1S)-1-(5-((5-chloro-4-fluoro-2,3-dihydro-1H-inden-2-yl)amino)pyridin-2-yl)-2,2,2-trifluoroethyl)-3-hydroxy-N-methylcyclobutane-1-carboxamide;
1-acetyl-N-((1S)-1-(5-((4,5-dichloro-2,3-dihydro-1H-inden-2-yl)amino)pyridin-2-yl)-2,2,2-trifluoroethyl)-N-methylazetidine-3-carboxamide;
N-((1S)-1-(5-((4,5-dichloro-2,3-dihydro-1H-inden-2-yl)amino)pyridin-2-yl)-2,2,2-trifluoroethyl)-1-(2-hydroxyacetyl)-N-methylazetidine-3-carboxamide;
N-((1S)-1-(5-((4,5-dichloro-2,3-dihydro-1H-inden-2-yl)amino)pyridin-2-yl)-2,2,2-trifluoroethyl)-N-methyl-2-(methylsulfonamido)acetamide;
(1,3-cis)-N-((1S)-1-(5-((5-chloro-4-fluoro-2,3-dihydro-1H-inden-2-yl)amino)pyridin-2-yl)-2,2,2-trifluoroethyl)-3-hydroxy-N-methylcyclobutane-1-carboxamide;
N-((1S)-1-(5-((5-chloro-4-fluoro-2,3-dihydro-1H-inden-2-yl)amino)pyridin-2-yl)-2,2,2-trifluoroethyl)-N-methyl-1-(oxetane-2-carbonyl)azetidine-3-carboxamide; and
(3S)—N-((1S)-1-(6-((5-chloro-4-fluoro-2,3-dihydro-1H-inden-2-yl)amino)pyridin-3-yl)-2,2,2-trifluoroethyl)-N-methyl-6-oxopiperidine-3-carboxamide.

16. A pharmaceutical composition comprising a compound according to any one of claims 1 to 15, or a pharmaceutically acceptable salt thereof, and one or more pharmaceutically acceptable excipients.

17. A compound according to any one of claims 1 to 15, or a pharmaceutically acceptable salt thereof, or a pharmaceutical composition according to claim 16, for use in therapy.

18. A compound according to any one of claims 1 to 15, or a pharmaceutically acceptable salt thereof, or a pharmaceutical composition according to claim 16, for use in the treatment of diseases or disorders mediated by MALT1.

19. A compound according to any one of claims 1 to 15, or a pharmaceutically acceptable salt thereof, or a pharmaceutical composition according to claim 16, for use in the treatment of non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukaemia (CLL), small lymphocytic lymphoma (SLL), Waldenström macroglobulinemia, lymphoblastic T cell leukaemia, chronic myelogenous leukaemia (CML), hairy-cell leukaemia, acute lymphoblastic T cell leukaemia, plasmacytoma, immunoblastic large cell leukaemia, megakaryoblastic leukaemia, acute megakaryocytic leukaemia, promyelocytic leukaemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, oesophageal cancer, salivary gland cancer, nasopharyngeal cancer, buccal cancer, cancer of the mouth, GIST (gastrointestinal stromal tumour), arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, or polymyositis.

20. A method of treating a disease or disorders mediated by MALT1, said method comprising administering to a subject in need of such treatment a therapeutically effective amount of a compound according to any one of claims 1 to 15, or a pharmaceutically acceptable salt thereof, or a pharmaceutical composition according to claim 16.

21. A method of treating non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukaemia (CLL), small lymphocytic lymphoma (SLL), Waldenström macroglobulinemia, lymphoblastic T cell leukaemia, chronic myelogenous leukaemia (CML), hairy-cell leukaemia, acute lymphoblastic T cell leukaemia, plasmacytoma, immunoblastic large cell leukaemia, megakaryoblastic leukaemia, acute megakaryocytic leukaemia, promyelocytic leukaemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, oesophageal cancer, salivary gland cancer, nasopharyngeal cancer, buccal cancer, cancer of the mouth, GIST (gastrointestinal stromal tumour), arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, or polymyositis, said method comprising administering to a subject in need of such treatment a therapeutically effective amount of a compound according to any one of claims 1 to 15, or a pharmaceutically acceptable salt thereof, or a pharmaceutical composition according to claim 16.
